SEQUENCE LISTING (4 sequences)

SEQ ID NO: 1 - protein, 12 aa, Artificial Sequence
  Synthetic construct
ELDLAVEFWHDR

SEQ ID NO: 2 - protein, 10 aa, Artificial Sequence
  Synthetic Construct
XEVNLDAEFX

SEQ ID NO: 3 - protein, 10 aa, Artificial Sequence
  Synthetic construct
SEVNLDAEFK

SEQ ID NO: 4 - protein, 10 aa, Artificial Sequence
  Synthetic Construct
XEVNLDAEFX

(19) United States
(12) Patent Application Publication
(10) Pub. No.: US 2010/0286145 A1
(43) Pub. Date: Nov. 11, 2010

(54) ISOPHTHALAMIDE DERIVATIVES INHIBITING BETA-SECRETASE ACTIVITY

(75) Inventors: Arun K. Ghosh, West Lafayette, IN (US); Chunfeng Liu, Norman, OK (US); Thippeswamy Devasamudram, Edmond, OK (US); Hui Lei, Edmond, OK (US); Lisa M. Swanson, Oklahoma City, OK (US); Sudha V. Ankala, Edmond, OK (US); John C. Lilly, Norman, OK (US); Geoffrey M. Bilcer, Edmond, OK (US)

Correspondence Address: MORRISON & FOERSTER LLP, 755 PAGE MILL RD, PALO ALTO, CA, 94304-1018, US

(73) Assignees: CoMentis, Inc., South San Francisco, CA (US); Purdue Research Foundation, West Lafayette, IN (US)

(21) Appl. No.: 12/670,105

(22) PCT Filed: Jul. 25, 2008
(86) PCT No.: PCT/US2008/071258

§ 371 Date: Apr. 23, 2010

Related U.S. Application Data
(60) Provisional application No. 60/952,198, filed on Jul. 26, 2007.
(60) Provisional application No. 60/980,735, filed on Oct. 17, 2007.

(51) Int. Cl. A61K 31/5377; C12N 9/99; A61K 31/4965; A61K 31/444; A61K 31/4439; A61K 31/427; C07D 417/12; C07D 417/14; A61P 25/28

(52) U.S. Cl. 514/235.5; 435/184; 514/255.05; 514/333; 514/342; 514/365; 544/133; 544/405; 546/256; 546/269.7; 548/204

(57) ABSTRACT

The present invention provides novel beta-secretase inhibitors and methods for their use, including methods of treating of Alzheimer's disease.

### BACKGROUND OF THE INVENTION

Alzheimer's disease is a progressive mental deterioration in a human resulting, inter alia, in loss of memory, confusion and disorientation. Alzheimer's disease accounts for the majority of senile dementias and is a leading cause of death in adults (Anderson, R. N., Natl. Vital Stat. Rep. 49:1-87 (2001), the teachings of which are incorporated herein in their entirety). Histologically, the brain of persons afflicted with Alzheimer's disease is characterized by a distortion of the intracellular neurofibrils and the presence of senile plaques composed of granular or filamentous argentophilic masses with an amyloid protein core, largely due to the accumulation of β-amyloid protein (Aβ) in the brain. Aβ accumulation plays a role in the pathogenesis and progression of the disease (Selkoe, D. J., Nature 399: 23-31 (1999)) and is a proteolytic fragment of amyloid precursor protein (APP). APP is cleaved initially by β-secretase followed by γ-secretase to generate Aβ (Lin, X., et al., Proc. Natl. Acad. Sci. USA 97:1456-1460 (2000); De Stropper, B., et al., Nature 391:387-390 (1998)). Inhibitors of β-secretase are described in U.S. Pat. No. 7,214,715, US 2007/0032470, WO 2006/110/668; WO 2002/02520; WO 2002/02505; WO 2002/02518; WO 2002/02512; WO 2003/040096; WO 2003/072535; WO 2003/050073; WO 2005/030709; WO 2004/050619; WO 2004/080376; WO 2004/043916; WO 2006/110668; Stachel, S. J., J. Med. Chem. 47, 6447-6450 (2004); Stachel, S. J., Bioorg. Med. Chem. Lett. 16, 641-644 (2006); and Varghese, J., Curr. Top. Med. Chem. 6: 569-578 (2006).

There is a need to develop effective compounds and methods for the treatment of Alzheimer's disease. The present invention fulfills these and other needs.

### BRIEF SUMMARY OF THE INVENTION

The present invention provides novel β-secretase inhibitor compounds and methods for their use, including methods of treating Alzheimer's disease.

In another aspect of the present invention, the β-secretase inhibitor compounds of the invention can be employed in methods to decrease memapsin 2 activity, decrease hydrolysis of a β-secretase site of a memapsin 2 substrate, and/or decrease the accumulation of β-secret protein relative to the amount of memapsin 2 activity, hydrolysis of a β-secretase site, and accumulation of β-secret protein, respectively, in the absence of the β-secretase inhibitor.

In another aspect, the present invention provides pharmaceutical compositions comprising a β-secretase inhibitor compound of the invention or a β-secretase inhibitor compound in combination with a pharmaceutically acceptable carrier.

In another aspect of the present invention, the β-secretase inhibitor compounds of the invention can be employed in the treatment of diseases or conditions associated with β-secretase activity, hydrolysis of a β-secretase site of a β-amyloid precursor protein, and/or β-amyloid protein accumulation. Typically, a mammal is treated for the disease or condition. In an exemplary embodiment, the disease is Alzheimer's disease.

In another aspect of the present invention, the β-secretase inhibitor compound includes any one, any combination, or all of the compounds of Example 3; or a stereoisomer, mixture of stereoisomers, crystalline form, non-crystalline form, hydrate, solvate, or salt thereof. In one embodiment, the β-secretase inhibitor compound includes any one, any combination, or all of the compounds of Example 3; or a pharmaceutically acceptable salt or solvate thereof. In some embodiments, the compound has a memapsin 2 Kᵢ of less than about 300 nM. In some embodiments, the compound has an apparent memapsin 2 Kᵢ of less than about 300 nM as measured by inhibition of memapsin 2 catalytic activity toward the fluorogenic substrate FS-2 (MCA-SEVNLDAEFR-DNP; SEQ ID NO.: 2). In some embodiments, the compound has a cellular Aβ production IC50 of less than about 1 μM. In some embodiments, the compound has a memapsin 1 Kᵢ and/or cathepsin D Kᵢ of greater than 300 nM. In some embodiments, the compound has an apparent memapsin 1 Kᵢ and/or apparent cathepsin D Kᵢ of greater than 300 nM, as measured by the substrate peptide NH₃-ELDLAVEFWHDR-CO₂ (SEQ ID NO.: 1). In some embodiments, the compound is capable of selectively reducing memapsin 2 catalytic activity relative to memapsin 1 catalytic activity. In some embodiments, the compound is capable of selectively reducing memapsin 2 catalytic activity relative to cathepsin D catalytic activity. In some of these embodiments, the relative reduction is greater than about 5-fold. In other embodiments, the reduction is greater than about 10-fold. In another embodiment, the β-secretase inhibitor compound (a) has a memapsin 2 Kᵢ of less than 300 nM (or less than 100 nM, or 10 nM); (b) has a cellular Aβ production IC50 of less than about 1 μM (or less than 300 nM, 100 nM, or 10 nM); and (c) is capable of selectively reducing memapsin 2 catalytic activity relative to memapsin 1 or cathepsin D catalytic activity by greater than about 5-fold (or greater than about 10-fold, or 100-fold). In some of these embodiments, the compound is capable of greater than about 25% (or great than about 40%, or about 50%) brain penetration in an individual (e.g., a human, monkey, dog or rat) relative to plasma after 24 hours post-administration.

In another aspect of the present invention, any one of the β-secretase inhibitor compounds is present in substantially pure form.

In another aspect of the present invention are provided formulations comprising any one of the compounds described herein and a pharmaceutically acceptable carrier. In some embodiments, the formulation is suitable for administration to an individual.

In another aspect of the present invention are provided formulations comprising an effective amount of any one of the compounds described herein and a pharmaceutically acceptable carrier.

In another aspect of the present invention are provided methods of treating Alzheimer's disease in an individual in need thereof, comprising administering to the individual an effective amount of any one of the compounds described herein.

In another aspect of the present invention are provided methods of reducing memapsin 2 catalytic activity, comprising contacting a memapsin 2 protein with an effective amount of any one of the compounds described herein. In some variations, the memapsin 2 beta-secretase is contacted in a cell. In some embodiments, the cell is contacted in vivo. In some embodiments, the cell is contacted in vitro.

In another aspect of the present invention are provided methods of selectively reducing memapsin 2 catalytic activity relative to memapsin 1 catalytic activity, comprising contacting a memapsin 2 protein with an effective amount of a compound of any one of the compounds described herein in the presence of memapsin 1 beta-secretase.

In another aspect of the present invention are provided methods of selectively reducing memapsin 2 catalytic activity relative to cathepsin D catalytic activity, comprising contacting a memapsin 2 protein with an effective amount of a compound of any one of the compounds described herein in the presence of cathepsin D.

In another aspect of the present invention are provided methods of selectively reducing memapsin 2 catalytic activity relative to memapsin 1 catalytic activity and cathepsin D catalytic activity, the method comprising contacting a memapsin 2 protein with an effective amount of a compound of any one of the compounds described herein in the presence of memapsin 1 beta-secretase and cathepsin D.

In another aspect of the present invention are provided methods of treating Glaucoma in an individual in need thereof, comprising administering to the individual an effective amount of any one of the compounds described herein.

In another aspect of the present invention is any one of the compounds described herein for use as a medicament.

Another aspect of the present invention is the use of any one of the compounds described herein for the manufacture of a medicament for the treatment or prevention of a condition characterized by memapsin 2 catalytic activity. In some variations, the condition is Alzheimer's disease.

In another aspect of the present invention are provided kits for the treatment or prevention in an individual with Alzheimer's disease, comprising any one of the compounds described herein; and packaging.

In another aspect of the present invention are provided kits for the treatment or prevention in an individual of a condition mediated by memapsin 2 catalytic activity, comprising any one of the compounds described herein; and packaging.

In another aspect of the present invention are provided kits for the treatment or prevention in an individual with Alzheimer's disease, comprising a formulation described herein; and packaging.

In another aspect of the present invention are provided kits for the treatment or prevention in an individual of a condition mediated by memapsin 2 catalytic activity, comprising a formulation described herein; and packaging.

### DETAILED DESCRIPTION OF THE INVENTION

### Abbreviations and Definitions

The abbreviations used herein have their conventional meaning within the chemical and biological arts, unless otherwise specified.

Nomenclature of some compounds described herein may be identified using ChemDraw Ultra Version 10.0, available from CambridgeSoft®.

The terms, “pharmaceutically effective amount,” “therapeutically effective amount,” “effective amount,” and cognates of these terms, as used herein refer to an amount that results in a desired pharmacological and/or physiological effect for a specified condition (e.g., disease, disorder, etc.) or one or more of its symptoms and/or to completely or partially prevent the occurrence of the condition or symptom thereof and/or may be therapeutic in terms of a partial or complete cure for the condition and/or adverse effect attributable to the condition. In reference to conditions mediated by memapsin 2 beta-secretase, a pharmaceutically or therapeutically effective amount comprises an amount sufficient to, among other things, cause antagonism of memapsin 2 beta-secretase. In reference to glaucoma, a pharmaceutically or therapeutically effective amount comprises an amount sufficient to, among other things, decrease intraocular pressure; and/or halt, reverse, and/or diminish the loss of retinal ganglion cells (RGCs). In certain embodiments, the pharmaceutically effective amount is sufficient to prevent the condition, as in being administered to an individual prophylactically.

The “pharmaceutically effective amount” or “therapeutically effective amount” will vary depending on the composition being administered, the condition being treated/prevented, the severity of the condition being treated or prevented, the age and relative health of the individual, the route and form of administration, the judgment of the attending medical or veterinary practitioner, and other factors appreciated by the skilled artisan in view of the teaching provided herein.

When used with respect to methods of treatment/prevention and the use of the polymorphs and compositions thereof described herein, an individual “in need thereof” may be an individual who has been diagnosed with or previously treated for the condition to be treated. With respect to prevention, the individual in need thereof may also be an individual who is at risk for a condition (e.g., a family history of the condition, life-style factors indicative of risk for the condition, etc.).

In some variations, the individual has been identified as having one or more of the conditions described herein. Identification of the conditions as described herein by a skilled physician is routine in the art and may also be suspected by the individual or others, for example, due to loss of memory in the case of Alzheimer's, exhibiting the symptoms of schizophrenia, etc., and due to loss of vision in the case of Glaucoma.

In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions), lifestyle or habits.

In some embodiments, the individual is a mammal, including, but not limited to, bovine, horse, feline, rabbit, canine, rodent, or primate. In some embodiments, the mammal is a primate. In some embodiments, the primate is a human. In some embodiments, the individual is human, including adults, children and premature infants. In some embodiments, the individual is a non-mammal. In some variations, the primate is a non-human primate such as chimpanzees and other apes and monkey species. In some embodiments, the mammal is a farm animal such as cattle, horses, sheep, goats, and swine; pets such as rabbits, dogs, and cats; laboratory animals including rodents, such as rats, mice, and guinea pigs; and the like. Examples of non-mammals include, but are not limited to, birds, and the like. The term “individual” does not denote a particular age or sex.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19, the content of which is hereby incorporated by reference in its entirety). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures), succinates, benzoates and salts with amino acids such as glutamic acid. For example, compounds described herein such as N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide, may exist as a citrate salt (e.g., mono citrate, hydrogen citrate, or dihydrogen citrate) and/or a mesylate salt (e.g., dimesylate). These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms (i.e., “solvates”). Compounds of the invention may also include hydrated forms (i.e., “hydrates”). In general, the solvated and hydrated forms are equivalent to unsolvated forms and are encompassed within the scope of the present invention. Certain compounds of the present invention may exist in multiple crystalline or amorphous forms (non-crystalline forms). In general, all physical forms are equivalent for the uses contemplated by the present invention and are intended to be within the scope of the present invention.

Metabolites of the compounds are also embraced by the invention. Metebolites may include primary metabolites and/or secondary metabolites. However, metabolites of substances which occur naturally in subjects are excluded from the claimed compounds of the invention.

As used herein, “isomer” includes all stereoisomers of the compounds referred to in the formulas herein, including enantiomers, diastereomers, as well as all conformers, rotomers, and tautomers. The invention includes all enantiomers of any chiral compound disclosed, in either substantially pure levorotatory or dextrorotatory form, or in a racemic mixture, or in any ratio of enantiomers. For compounds disclosed as an (R)-enantiomer, the invention also includes the (S)-enantiomer; for compounds disclosed as the (S)-enantiomer, the invention also includes the (R)-enantiomer. The invention includes any diastereomers of the compounds referred to in the above formulas in diastereomerically pure form and in the form of mixtures in all ratios.

Unless stereochemistry is explicitly indicated in a chemical structure or chemical name, the chemical structure or chemical name is intended to embrace all possible stereoisomers, conformers, rotomers, and tautomers of the compound depicted. For example, a compound containing a chiral carbon atom is intended to embrace both the (R) enantiomer and the (S) enantiomer. A compound containing multiple chiral carbon atoms (for example, both carbons within the hydroxyethylamine isostere) is intended to embrace all enantiomers and diastereomers (including (R,R), (S,S), (R,S), and (R,S) isomers). When a compound is explicitly indicated in a particular stereochemical arrangement (e.g., 2S,3R for the hydroxyethylamine isostere), then the compound may, in other embodiments, be described in another specific stereochemical arrangement (e.g., 2R,3S for the hydroxyethylamine isostere) and/or a mixture of stereochemical arrangements.

A substantially pure compound means that the compound is present with no more than 15% or no more than 10% or no more than 5% or no more than 3% or no more than 1% of the total amount of compound in a different stereochemical form. For instance, substantially pure S,S compound means that no more than 15% or no more than 10% or no more than 5% or no more than 3% or no more than 1% of the total R,R; S,R; and R,S form is present.

A composition may contain the compound as mixtures of such stereoisomers, where the mixture may be enanteomers (e.g., S,S and R,R) or diastereomers (e.g., S,S and R,S or S,R) in equal or unequal amounts. A composition may contain the compound as a mixture of 2 or 3 or 4 such stereoisomers in any ratio of stereoisomers.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

A “transition state isostere,” or “isostere,” as used herein, is a compound comprising the hydroxyethylamine linking group —CH(OH)—CH₂—NH—. This isostere is also referred to herein as a “hydroxyethylamine isostere.” The hydroxyethylamine linking group may be found between a pair of natural or non-natural amino acids of a peptide. A hydroxyethylamine group is an isostere of the transition state of hydrolysis of an amide bond.

“Amyloid precursor protein,” or “APP,” as used herein, refers to a β-secret precursor comprising a β-secretase site.

“Memapsin-2,” as used herein, refers to proteins identified by National Center for Biotechnology Information (“NCBI”) accession number NP_{—}036236 (sometimes referred to as “β-site APP-cleaving enzyme 1” or “BACE-1”), including homologs, isoforms and subdomains thereof that retain proteolytic activity. Sequence identities of active memapsin 2 proteins and protein fragments (and nucleic acid coding sequences thereof) have been previously disclosed and discussed in detail in U.S. Application No. 20040121947, and International Application No. PCT/USO2/34324 (Publication No. WO 03/039454), which are herein incorporated by reference for all purposes in their entirety.

“Memapsin-1,” as used herein, refers to proteins identified by National Center for Biotechnology Information (“NCBI”) accession number NP_{—}036237 (sometimes referred to as “β-site APP-cleaving enzyme 2” or “BACE-2”) and/or those previously disclosed and discussed in detail in see U.S. Patent Application Publication No. 20040121947, and International Application No. PCT/USO2/34324 (Publication No. WO 03/039454), incorporated by reference herein in their entirety for all purposes, including homologs, isoforms and subdomains thereof that retain proteolytic activity.

“Cathepsin D,” as used herein, refers to proteins identified by National Center for Biotechnology Information (“NCBI”) accession number NP_{—}036236 (sometimes referred to as “β-site APP-cleaving enzyme 1” or “BACE-1”) and or proteins identified by Enzyme Structure Database subclass EC 3.4.23.5., including homologs, isoforms and subdomains thereof that retain proteolytic activity.

A “β-secretase site” is an amino acid sequence that is cleaved by an active memapsin 2 or active fragment thereof. Specific β-secretase sites have also been previously set forth and discussed in detail in U.S. Application No. 20040121947, and International Application No. PCT/USO2/34324 (Publication No. WO 03/039454), which are herein incorporated by reference for all purposes in their entirety, and include the Swedish mutation sequence, and the native β-amyloid precursor protein cleavage sequence. Thus, β-secretase inhibitors may be tested for their ability to decrease the hydrolysis of the β-secretase site of a substrate, such as the β-amyloid precursor protein, analogs of β-amyloid precursor protein, or fragments of β-amyloid precursor protein.

A “beta-secretase inhibitor” (i.e. β-secretase inhibitor) refers to a compound capable of reducing the proteolytic activity of memapsin-2 relative to the activity in the absence of inhibitor.

The terms “a” or “an,” as used in herein means one or more.

### I. β-SECRETASE INHIBITORS

In one aspect, the present invention provides compounds that inhibit (i.e. decrease) the catalytic activity of the β-secretase enzyme (memapsin 2). These compounds may be referred to herein as “compounds of the present invention,” “β-secretase inhibitor compounds,” or “memapsin 2 β-secretase inhibitors.”

In this aspect, the β-secretase inhibitor compound is any one of the compounds of Table 1.

In some embodiments, the β-secretase inhibitor compound is: N1-((2S,3R)-3-hydroxy-4-(3-hydroxybenzylamino)-1-phenylbutan-2-yl)-5-(N-methylmethylsulfonamido)-N3-((R)-1-phenylethyl)isophthalamide; N1-(2S,3R)-4-(3-tert-butyl-2-hydroxybenzylamino)-3-hydroxy-1-phenylbutan-2-yl)-5-(N-methylmethylsulfonamido)-N3-((R)-1-phenylethyl)isophthalamide; N1-(2S,3R)-4-(5-tert-butyl-2-(tert-butyldimethylsilyloxy)benzylamino)-3-hydroxy-1-phenylbutan-2-yl)-5-(N-methylmethylsulfonamido)-N3-((R)-1-phenylethyl)isophthalamide; N1-((2S,3R)-4-(5-tert-butyl-2-hydroxybenzylamino)-3-hydroxy-1-phenylbutan-2-yl)-5-(N-methylmethylsulfonamido)-N3-((R)-1-phenylethyl)isophthalamide; N1-((2S,3R)-4-(3-acetylbenzylamino)-3-hydroxy-1-phenylbutan-2-yl)-5-(N-methylmethylsulfonamido)-N3-((R)-1-phenylethyl)isophthalamide; N1-((2S,3R)-4-(3-chlorobenzylamino)-3-hydroxy-1-phenylbutan-2-yl)-5-(N-methylmethylsulfonamido)-N3-((R)-1-phenylethyl)isophthalamide; N1-((2S,3R)-3-hydroxy-4-(3-iodobenzylamino)-1-phenylbutan-2-yl)-5-(N-methylmethylsulfonamido)-N3-((R)-1-phenylethyl)isophthalamide; methyl 3-(((2R,3S)-2-hydroxy-3-(3-(N-methylmethylsulfonamido)-5-((R)-1-phenylethylcarbamoyl)benzamido)-4-phenylbutylamino)methyl)benzoate; N1-((2S,3R)-3-hydroxy-4-(3-(hydroxymethyl)benzylamino)-1-phenylbutan-2-yl)-5-(N-methylmethylsulfonamido)-N3-((R)-1-phenylethyl)isophthalamide; 3-(((2R,3S)-2-hydroxy-3-(3-(N-methylmethylsulfonamido)-5-((R)-1-phenylethylcarbamoyl)benzamido)-4-phenylbutylamino)methyl)benzoic acid; methyl 3-(((2R,3S)-2-hydroxy-3-(3-(N-methylmethylsulfonamido)-5-((R)-1-phenylethylcarbamoyl)benzamido)-4-phenylbutylamino)methyl)-5-methoxybenzoate; N1-((2S,3R)-4-(3-tert-butylbenzylamino)-3-hydroxy-1-phenylbutan-2-yl)-5-(N-methylmethylsulfonamido)-N3-((R)-1-phenylethyl)isophthalamide; N1-((2S,3R)-4-((5-(2-fluoropropan-2-yl)pyridin-3-yl)methylamino)-3-hydroxy-1-phenylbutan-2-yl)-5-(N-methylmethylsulfonamido)-N3-((R)-1-phenylethyl)isophthalamide; N1-((2S,3R)-3-hydroxy-1-phenyl-4-((5-(trifluoromethyl)pyridin-3-yl)methylamino)butan-2-yl)-5-(N-methylmethylsulfonamido)-N3-((R)-1-phenylethyl)isophthalamide; N1-((2S,3R)-4-((1-tert-butyl-5-methyl-1H-pyrazol-4-yl)methylamino)-3-hydroxy-1-phenylbutan-2-yl)-5-(N-methylmethylsulfonamido)-N3-((R)-1-phenylethyl)isophthalamide; N1-((2S,3R)-3-hydroxy-4-((1-(2-methoxyethyl)-5-methyl-1H-pyrazol-4-yl)methylamino)-1-phenylbutan-2-yl)-5-(N-methylmethylsulfonamido)-N3-((R)-1-phenylethyl)isophthalamide; N1-((2S,3R)-3-hydroxy-4-(3-methoxybenzylamino)-1-phenylbutan-2-yl)-5-(N-methylmethylsulfonamido)-N3-((6-methylpyridin-2-yl)methyl)isophthalamide; N1-((2S,3R)-3-hydroxy-4-(3-methoxybenzylamino)-1-phenylbutan-2-yl)-5-(N-methylmethylsulfonamido)-N3-((4-methylpyrimidin-2-yl)methyl)isophthalamide; N1-((2S,3R)-3-hydroxy-1-phenyl-4-((S)-1-phenylethylamino)butan-2-yl)-5-(N-methylmethylsulfonamido)-N3-(1-(5-methylthiophen-2-yl)ethyl)isophthalamide; N1-((2S,3R)-3-hydroxy-4-(methyl((S)-1-phenylethyl)amino)-1-phenylbutan-2-yl)-N3-((R)-1-(3-methoxyphenyl)ethyl)-5-(N-methylmethylsulfonamido)isophthalamide; N1-((2S,3R)-3-hydroxy-4-(methyl((S)-1-phenylethyl)amino)-1-phenylbutan-2-yl)-N3-(1-(5-methylfuran-2-yl)ethyl)-5-(N-methylmethylsulfonamido)isophthalamide; N1-((2S,3R)-3-hydroxy-4-(methyl((S)-1-phenylethyl)amino)-1-phenylbutan-2-yl)-5-(N-methylmethylsulfonamido)-N3-(1-(5-methylthiophen-2-yl)ethyl)isophthalamide; N1-((2S,3R)-3-hydroxy-4-(methyl((S)-1-phenylethyl)amino)-1-phenylbutan-2-yl)-5-(N-methylmethylsulfonamido)-N3-(1-(3-methylthiophen-2-yl)ethyl)isophthalamide; N1-((2S,3R)-3-hydroxy-1-phenyl-4-((S)-1-phenylethylamino)butan-2-yl)-N3-methyl-5-(N-methylmethylsulfonamido)-N3-((S)-1-phenylethyl)isophthalamide; N1-((2S,3R)-3-hydroxy-1-phenyl-4-((S)-1-phenylethylamino)butan-2-yl)-N3-((R)-1-(3-methoxyphenyl)ethyl)-5-(N-methylmethylsulfonamido)isophthalamide; N1-((2S,3R)-3-hydroxy-1-phenyl-4-((S)-1-phenylethylamino)butan-2-yl)-N3-(1-(5-methylfuran-2-yl)ethyl)-5-(N-methylmethylsulfonamido)isophthalamide; N1-((2S,3R)-3-hydroxy-1-phenyl-4-((S)-1-phenylethylamino)butan-2-yl)-5-(N-methylmethylsulfonamido)-N3-(1-o-tolylethyl)isophthalamide; N1-((2S,3R)-3-hydroxy-1-phenyl-4-((R)-1-p-tolylethylamino)butan-2-yl)-5-(N-methylmethylsulfonamido)-N3-((S)-1-phenylethyl)isophthalamide; N1-((2S,3R)-3-hydroxy-1-phenyl-4-((R)-1-p-tolylethylamino)butan-2-yl)-N3-((R)-1-(4-methoxyphenyl)ethyl)-5-(N-methylmethylsulfonamido)isophthalamide; N1-((2S,3R)-3-hydroxy-1-phenyl-4-((R)-1-p-tolylethylamino)butan-2-yl)-N3-((R)-1-(3-methoxyphenyl)ethyl)-5-(N-methylmethylsulfonamido)isophthalamide; N1-((2S,3R)-3-hydroxy-4-((R)-1-(4-methoxyphenyl)ethylamino)-1-phenylbutan-2-yl)-5-(N-methylmethylsulfonamido)-N3-((R)-1-p-tolylethyl)isophthalamide; N1-((2S,3R)-3-hydroxy-4-((R)-1-(4-methoxyphenyl)ethylamino)-1-phenylbutan-2-yl)-N3-((R)-1-(4-methoxyphenyl)ethyl)-5-(N-methylmethylsulfonamido)isophthalamide; N1-((2S,3R)-3-hydroxy-4-((R)-1-(4-methoxyphenyl)ethylamino)-1-phenylbutan-2-yl)-5-(N-methylmethylsulfonamido)-N3-(1-(5-methylthiophen-2-yl)ethyl)isophthalamide; N1-((2S,3R)-3-hydroxy-4-((R)-1-(4-methoxyphenyl)ethylamino)-1-phenylbutan-2-yl)-5-(N-methylmethylsulfonamido)-N3-(1-(3-methylthiophen-2-yl)ethyl)isophthalamide; N1-((2S,3R)-3-hydroxy-4-((R)-1-(3-methoxyphenyl)ethylamino)-1-phenylbutan-2-yl)-N3-methyl-5-(N-methylmethylsulfonamido)-N3-((S)-1-phenylethyl)isophthalamide; N1-((2S,3R)-3-hydroxy-4-((R)-1-(3-methoxyphenyl)ethylamino)-1-phenylbutan-2-yl)-N3-((R)-1-(4-methoxyphenyl)ethyl)-5-(N-methylmethylsulfonamido)isophthalamide; N1-(1-(furan-2-yl)ethyl)-N3-((2S,3R)-3-hydroxy-4-((R)-1-(3-methoxyphenyl)ethylamino)-1-phenylbutan-2-yl)-5-(N-methylmethylsulfonamido)isophthalamide; N1-(1-(furan-2-yl)ethyl)-N3-((2S,3R)-3-hydroxy-4-(methyl((S)-1-phenylethyl)amino)-1-phenylbutan-2-yl)-5-(N-methylmethylsulfonamido)isophthalamide; N1-((2S,3R)-3-hydroxy-1-phenyl-4-((R)-1-p-tolylethylamino)butan-2-yl)-5-(N-methylmethylsulfonamido)-N3-((R)-1-p-tolylethyl)isophthalamide; N1-(1-(furan-2-yl)ethyl)-N3-((2S,3R)-3-hydroxy-1-phenyl-4-((R)-1-p-tolylethylamino)butan-2-yl)-5-(N-methylmethylsulfonamido)isophthalamide; N1-((2S,3R)-3-hydroxy-1-phenyl-4-((R)-1-p-tolylethylamino)butan-2-yl)-5-(N-methylmethylsulfonamido)-N3-(1-o-tolylethyl)isophthalamide; N1-((2S,3R)-3-hydroxy-4-((R)-1-(3-methoxyphenyl)ethylamino)-1-phenylbutan-2-yl)-N3-((R)-1-(4-methoxyphenyl)ethyl)-5-(N-methylmethylsulfonamido)isophthalamide; N1-((2S,3R)-3-hydroxy-4-((R)-1-(3-methoxyphenyl)ethylamino)-1-phenylbutan-2-yl)-5-(N-methylmethylsulfonamido)-N3-(1-o-tolylethyl)isophthalamide; N1-((2S,3R)-3-hydroxy-1-phenyl-4-((R)-1-p-tolylethylamino)butan-2-yl)-5-(N-methylmethylsulfonamido)-N3-(1-(3-methylthiophen-2-yl)ethyl)isophthalamide; N1-((2S,3R)-3-hydroxy-4-((R)-1-(4-methoxyphenyl)ethylamino)-1-phenylbutan-2-yl)-N3-methyl-5-(N-methylmethylsulfonamido)-N3-((S)-1-phenylethyl)isophthalamide; N1-((2S,3R)-3-hydroxy-1-phenyl-4-((R)-1-p-tolylethylamino)butan-2-yl)-5-(N-methylmethylsulfonamido)-N3-(1-(5-methylthiophen-2-yl)ethyl)isophthalamide; N1-((2S,3R)-3-hydroxy-4-((R)-1-(4-methoxyphenyl)ethylamino)-1-phenylbutan-2-yl)-5-(N-methylmethylsulfonamido)-N3-((S)-1-phenylethyl)isophthalamide; N1-(1-(furan-2-yl)ethyl)-N3-((2S,3R)-3-hydroxy-4-((R)-1-(4-methoxyphenyl)ethylamino)-1-phenylbutan-2-yl)-5-(N-methylmethylsulfonamido)isophthalamide; N1-((2S,3R)-3-hydroxy-1-phenyl-4-((S)-1-phenylethylamino)butan-2-yl)-N3-((R)-1-(4-methoxyphenyl)ethyl)-5-(N-methylmethylsulfonamido)isophthalamide; N1-((2S,3R)-3-hydroxy-4-((R)-1-(3-methoxyphenyl)ethylamino)-1-phenylbutan-2-yl)-5-(N-methylmethylsulfonamido)-N3-(1-(3-methylthiophen-2-yl)ethyl)isophthalamide; N1-((2S,3R)-4-(1-(furan-2-yl)ethylamino)-3-hydroxy-1-phenylbutan-2-yl)-5-(N-methylmethylsulfonamido)-N3-(1-(3-methylthiophen-2-yl)ethyl)isophthalamide; N1-((2S,3R)-3-hydroxy-1-phenyl-4-((R)-1-p-tolylethylamino)butan-2-yl)-N3-methyl-5-(N-methylmethylsulfonamido)-N3-((S)-1-phenylethyl)isophthalamide; N1-((2S,3R)-3-hydroxy-4-((R)-1-(4-methoxyphenyl)ethylamino)-1-phenylbutan-2-yl)-N3-((R)-1-(3-methoxyphenyl)ethyl)-5-(N-methylmethylsulfonamido)isophthalamide; N1-((2S,3R)-3-hydroxy-4-((R)-1-(3-methoxyphenyl)ethylamino)-1-phenylbutan-2-yl)-5-(N-methylmethylsulfonamido)-N3-(1-(5-methylthiophen-2-yl)ethyl)isophthalamide; N1-((2S,3R)-4-(1-(furan-2-yl)ethylamino)-3-hydroxy-1-phenylbutan-2-yl)-N3-((R)-1-(4-methoxyphenyl)ethyl)-5-(N-methylmethylsulfonamido)isophthalamide; N1-((2S,3R)-4-(1-(furan-2-yl)ethylamino)-3-hydroxy-1-phenylbutan-2-yl)-5-(N-methylmethylsulfonamido)-N3-((S)-1-phenylethyl)isophthalamide; N1-((2S,3R)-3-hydroxy-1-phenyl-4-((S)-1-phenylethylamino)butan-2-yl)-5-(N-methylmethylsulfonamido)-N3-((R)-1-p-tolylethyl)isophthalamide; N1-(1-(furan-2-yl)ethyl)-N3-((2S,3R)-3-hydroxy-1-phenyl-4-((S)-1-phenylethylamino)butan-2-yl)-5-(N-methylmethylsulfonamido)isophthalamide; N1-((2S,3R)-3-hydroxy-1-phenyl-4-((S)-1-phenylethylamino)butan-2-yl)-5-(N-methylmethylsulfonamido)-N3-(1-(3-methylthiophen-2-yl)ethyl)isophthalamide; N1-((2S,3R)-3-hydroxy-4-(methyl((S)-1-phenylethyl)amino)-1-phenylbutan-2-yl)-5-(N-methylmethylsulfonamido)-N3-((R)-1-p-tolylethyl)isophthalamide; N1-((2S,3R)-3-hydroxy-4-(methyl((S)-1-phenylethyl)amino)-1-phenylbutan-2-yl)-N3-(1-(4-methoxyphenyl)ethyl)-N3-methyl-5-(N-methylmethylsulfonamido)isophthalamide; N1-((2S,3R)-3-hydroxy-1-phenyl-4-((S)-1-phenylethylamino)butan-2-yl)-5-(N-methylmethylsulfonamido)-N3-((S)-1-phenylethyl)isophthalamide; N1-((2S,3R)-4-(1-(furan-2-yl)ethylamino)-3-hydroxy-1-phenylbutan-2-yl)-5-(N-methylmethylsulfonamido)-N3-(1-(5-methylthiophen-2-yl)ethyl)isophthalamide; N1-((2S,3R)-3-hydroxy-4-((R)-1-(4-methoxyphenyl)ethylamino)-1-phenylbutan-2-yl)-N3-(1-(5-methylfuran-2-yl)ethyl)-5-(N-methylmethylsulfonamido)isophthalamide; N1-((2S,3R)-4-((1-tert-butyl-1H-pyrazol-4-yl)methylamino)-3-hydroxy-1-phenylbutan-2-yl)-5-(N-methylmethylsulfonamido)-N3-((R)-1-phenylethyl)isophthalamide; methyl 3-(((2R,3S)-2-hydroxy-3-(3-(methyl((4-methylthiazol-2-yl)methyl)carbamoyl)benzamido)-4-phenylbutylamino)methyl)benzoate; N1-((2S,3R)-4-(3-(1,1-difluoroethyl)benzylamino)-3-hydroxy-1-phenylbutan-2-yl)-5-(N-methylmethylsulfonamido)-N3-((R)-1-phenylethyl)isophthalamide; N1-((2S,3R)-3-hydroxy-4-((R)-1-(3-methoxyphenyl)ethylamino)-1-phenylbutan-2-yl)-5-(N-methylmethylsulfonamido)-N3-((R)-1-phenylethyl)isophthalamide; N1-((2S,3R)-4-(3-cyanobenzylamino)-3-hydroxy-1-phenylbutan-2-yl)-5-(N-methylmethylsulfonamido)-N3-((R)-1-phenylethyl)isophthalamide; or a stereoisomer, mixture of stereoisomers, crystalline form, non-crystalline form, hydrate, solvate, or salt thereof.

In other embodiments, the β-secretase inhibitor compound is: N¹-((2S,3R)-3-hydroxy-4-((3-methoxybenzyl)(methyl)amino)-1-phenylbutan-2-yl)-N3-methyl-N3-((4-methylthiazol-2-yl)methyl)isophthalamide; N1-((2S,3R)-4-(3-(dimethylamino)benzylamino)-3-hydroxy-1-phenylbutan-2-yl)-N3-methyl-N3-((4-methylthiazol-2-yl)methyl)isophthalamide; N1-((2S,3R)-4-(3-fluoro-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-4-(2-fluoro-6-methoxybenzylamino)-3-hydroxy-1-phenylbutan-2-yl)-N3-methyl-N3-((4-methylthiazol-2-yl)methyl)isophthalamide; N1-((2S,3R)-4-(3-tert-butyl-2-hydroxybenzylamino)-3-hydroxy-1-phenylbutan-2-yl)-N3-methyl-N3-((4-methylthiazol-2-yl)methyl)isophthalamide; N1-((2S,3R)-4-(5-tert-butyl-2-hydroxybenzylamino)-3-hydroxy-1-phenylbutan-2-yl)-N3-methyl-N3-((4-methylthiazol-2-yl)methyl)isophthalamide; N1-((2S,3R)-4-(3-acetylbenzylamino)-3-hydroxy-1-phenylbutan-2-yl)-N3-methyl-N3-((4-methylthiazol-2-yl)methyl)isophthalamide; N1-((2S,3R)-4-(3,5-dimethoxybenzylamino)-3-hydroxy-1-phenylbutan-2-yl)-N3-methyl-N3-((4-methylthiazol-2-yl)methyl)isophthalamide; N1-((2S,3R)-4-(3,4-dimethylbenzylamino)-3-hydroxy-1-phenylbutan-2-yl)-N3-methyl-N3-((4-methylthiazol-2-yl)methyl)isophthalamide; N1-((2S,3R)-4-(3,5-bis(trifluoromethyl)benzylamino)-3-hydroxy-1-phenylbutan-2-yl)-N3-methyl-N3-((4-methylthiazol-2-yl)methyl)isophthalamide; methyl 3-(((2R,3S)-2-hydroxy-3-(3-(methyl((4-methylthiazol-2-yl)methyl)carbamoyl)benzamido)-4-phenylbutylamino)methyl)-5-methoxybenzoate; 3-(((2R,3S)-2-hydroxy-3-(3-(methyl((4-methylthiazol-2-yl)methyl)carbamoyl)benzamido)-4-phenylbutylamino)methyl)-5-methoxybenzoic acid; N1-((2S,3R)-3-hydroxy-4-(3-iodobenzylamino)-1-phenylbutan-2-yl)-N3-methyl-N3-((4-methylthiazol-2-yl)methyl)isophthalamide; N1-((2S,3R)-4-(3-chlorobenzylamino)-3-hydroxy-1-phenylbutan-2-yl)-N3-methyl-N3-((4-methylthiazol-2-yl)methyl)isophthalamide; N1-((2S,3R)-3-hydroxy-4-(3-methoxy-5-(methylcarbamoyl)benzylamino)-1-phenylbutan-2-yl)-N3-methyl-N3-((4-methylthiazol-2-yl)methyl)isophthalamide; benzyl 3-(((2R,3S)-2-hydroxy-3-(3-(methyl((4-methylthiazol-2-yl)methyl)carbamoyl)benzamido)-4-phenylbutylamino)methyl)-5-isopropylphenylcarbamate; N1-((2S,3R)-4-(3-amino-5-isopropylbenzylamino)-3-hydroxy-1-phenylbutan-2-yl)-N3-methyl-N3-((4-methylthiazol-2-yl)methyl)isophthalamide; N1-((2S,3R)-4-(3-(dimethylamino)-5-isopropylbenzylamino)-3-hydroxy-1-phenylbutan-2-yl)-N3-methyl-N3-((4-methylthiazol-2-yl)methyl)isophthalamide; N1-((2S,3R)-4-(3-(benzyloxy)-5-isopropylbenzylamino)-3-hydroxy-1-phenylbutan-2-yl)-N3-methyl-N3-((4-methylthiazol-2-yl)methyl)isophthalamide; N1-((2S,3R)-3-hydroxy-4-(3-isopropyl-5-(methylsulfonamido)benzylamino)-1-phenylbutan-2-yl)-N3-methyl-N3-((4-methylthiazol-2-yl)methyl)isophthalamide; N1-((2S,3R)-3-hydroxy-4-(3-hydroxy-5-isopropylbenzylamino)-1-phenylbutan-2-yl)-N3-methyl-N3-((4-methylthiazol-2-yl)methyl)isophthalamide; benzyl 3-(((2R,3S)-2-hydroxy-3-(3-(methyl((4-methylthiazol-2-yl)methyl)carbamoyl)benzamido)-4-phenylbutylamino)methyl)-5-isopropylphenyl(methyl)carbamate; N1-((2S,3R)-3-hydroxy-4-(3-isopropyl-5-(methylamino)benzylamino)-1-phenylbutan-2-yl)-N3-methyl-N3-((4-methylthiazol-2-yl)methyl)isophthalamide; methyl 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoate; N1-((2S,3R)-3-hydroxy-4-(3-hydroxybenzylamino)-1-phenylbutan-2-yl)-N3-methyl-N3-((4-methylthiazol-2-yl)methyl)isophthalamide; N1-((2S,3R)-4-(3-tert-butylbenzylamino)-3-hydroxy-1-phenylbutan-2-yl)-N3-methyl-N3-((4-methylthiazol-2-yl)methyl)isophthalamide; N1-((2S,3R)-4-(3-cyclopropylbenzylamino)-3-hydroxy-1-phenylbutan-2-yl)-N3-methyl-N3-((4-methylthiazol-2-yl)methyl)isophthalamide; N1-((2S,3R)-3-hydroxy-4-(naphthalen-1-ylmethylamino)-1-phenylbutan-2-yl)-N3-methyl-N3-((4-methylthiazol-2-yl)methyl)isophthalamide; N1-((2S,3R)-3-hydroxy-1-phenyl-4-(pyridin-3-ylmethylamino)butan-2-yl)-N3-methyl-N3-((4-methylthiazol-2-yl)methyl)isophthalamide; N1-((2S,3R)-3-hydroxy-1-phenyl-4-(pyridin-4-ylmethylamino)butan-2-yl)-N3-methyl-N3-((4-methylthiazol-2-yl)methyl)isophthalamide; N1-((2S,3R)-3-hydroxy-1-phenyl-4-(pyridin-2-ylmethylamino)butan-2-yl)-N3-methyl-N3-((4-methylthiazol-2-yl)methyl)isophthalamide; N1-((2S,3R)-3-hydroxy-1-phenyl-4-(1-(pyridin-3-yl)ethylamino)butan-2-yl)-N3-methyl-N3-((4-methylthiazol-2-yl)methyl)isophthalamide; N1-((2S,3R)-3-hydroxy-4-((6-methylpyridin-2-yl)methylamino)-1-phenylbutan-2-yl)-N3-methyl-N3-((4-methylthiazol-2-yl)methyl)isophthalamide; N1-((2R,3S)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-1-phenyl-4-((6-(trifluoromethyl)pyridin-3-yl)methylamino)butan-2-yl)-N3-methyl-N3-((4-methylthiazol-2-yl)methyl)isophthalamide; N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((4-isopropylpyridin-2-yl)methylamino)-1-phenylbutan-2-yl)-N3-methyl-N3-((4-methylthiazol-2-yl)methyl)isophthalamide; N1-((2S,3R)-3-hydroxy-4-((6-isopropylpyridin-2-yl)methylamino)-1-phenylbutan-2-yl)-N3-methyl-N3-((4-methylthiazol-2-yl)methyl)isophthalamide; N1-((2S,3R)-3-hydroxy-4-((2-isopropylpyridin-4-yl)methylamino)-1-phenylbutan-2-yl)-N3-methyl-N3-((4-methylthiazol-2-yl)methyl)isophthalamide; methyl 6-(((2R,3S)-2-hydroxy-3-(3-(methyl((4-methylthiazol-2-yl)methyl)carbamoyl)benzamido)-4-phenylbutylamino)methyl)-2-methylnicotinate; N1-((2S,3R)-3-hydroxy-4-((5-(hydroxymethyl)-6-methylpyridin-2-yl)methylamino)-1-phenylbutan-2-yl)-N3-methyl-N3-((4-methylthiazol-2-yl)methyl)isophthalamide; N1-((2S,3R)-4-((5-(dimethylamino)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((2-fluoro-5-isopropylpyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((5-isopentylpyridin-3-yl)methylamino)-1-phenylbutan-2-yl)-N3-methyl-N3-((4-methylthiazol-2-yl)methyl)isophthalamide; N1-((2S,3R)-3-hydroxy-4-((6-methyl-5-(methylthiomethyl)pyridin-2-yl)methylamino)-1-phenylbutan-2-yl)-N3-methyl-N3-((4-methylthiazol-2-yl)methyl)isophthalamide; N1-((2S,3R)-4-((2,6-diisopropylpyridin-4-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((R)-1-(4-methylthiazol-2-yl)ethyl)isophthalamide; N1-((2S,3R)-4-((5-cyclopropylpyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-((5-(prop-1-en-2-yl)pyridin-3-yl)methylamino)butan-2-yl)-N3-methyl-N3-((4-methylthiazol-2-yl)methyl)isophthalamide; N1-((2S,3R)-4-((1-tert-butyl-5-methyl-1H-pyrazol-4-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((1-(2-methoxyethyl)-5-methyl-1H-pyrazol-4-yl)methylamino)-1-phenylbutan-2-yl)-N3-methyl-N3-((4-methylthiazol-2-yl)methyl)isophthalamide; N1-((2S,3R)-3-hydroxy-4-((4-methoxypyrimidin-2-yl)methylamino)-1-phenylbutan-2-yl)-N3-methyl-N3-((4-methylthiazol-2-yl)methyl)isophthalamide; N1-((2S,3R)-4-((1H-indol-7-yl)methylamino)-3-hydroxy-1-phenylbutan-2-yl)-N3-methyl-N3-((4-methylthiazol-2-yl)methyl)isophthalamide; N1-cyclopropyl-N3-((2S,3R)-3-hydroxy-1-phenyl-4-(3-(trifluoromethyl)benzylamino)butan-2-yl)-N1-((4-methylthiazol-2-yl)methyl)isophthalamide; N1-cyclopropyl-N3-((2S,3R)-3-hydroxy-1-phenyl-4-(3-(trifluoromethoxy)benzylamino)butan-2-yl)-N1-((4-methylthiazol-2-yl)methyl)isophthalamide; N1-((2S,3R)-3-hydroxy-4-(3-methoxybenzylamino)-1-phenylbutan-2-yl)-N3-((4-methylthiazol-2-yl)methyl)-N3-propylisophthalamide; N1-cyclopropyl-N3-((2S,3R)-3-hydroxy-1-phenyl-4-((5-(trifluoromethyl)pyridin-3-yl)methylamino)butan-2-yl)-N1-((4-methylthiazol-2-yl)methyl)isophthalamide; N1-((2S,3R)-3-hydroxy-4-((5-isopropyl-2,6-dimethylpyridin-3-yl)methylamino)-1-phenylbutan-2-yl)-N3-((4-methylthiazol-2-yl)methyl)isophthalamide; N1-((2S,3R)-3-hydroxy-4-((6-methyl-5-(methylsulfonylmethyl)pyridin-2-yl)methylamino)-1-phenylbutan-2-yl)-N3-((4-methylthiazol-2-yl)methyl)isophthalamide; N1-((4,5-dimethylthiazol-2-yl)methyl)-N3-((2S,3R)-3-hydroxy-4-(3-methoxybenzylamino)-1-phenylbutan-2-yl)-N1-methylisophthalamide; N1-((2S,3R)-3-hydroxy-4-(3-methoxybenzylamino)-1-phenylbutan-2-yl)-N3-methyl-N3-((5-methylthiazol-2-yl)methyl)isophthalamide; N1-((2S,3R)-3-hydroxy-4-(3-methoxybenzylamino)-1-phenylbutan-2-yl)-N3-(4-methylthiazol-2-yl)isophthalamide; N1-((2S,3R)-3-hydroxy-4-(3-methoxybenzylamino)-1-phenylbutan-2-yl)-N3-methyl-N3-(4-methylthiazol-2-yl)isophthalamide; N1-((2S,3R)-3-hydroxy-4-(3-methoxybenzylamino)-1-phenylbutan-2-yl)-N3-methyl-N3-((1-methyl-1H-pyrazol-3-yl)methyl)isophthalamide; N1-(furan-2-ylmethyl)-N3-((2S,3R)-3-hydroxy-4-(3-methoxybenzylamino)-1-phenylbutan-2-yl)-N1-methylisophthalamide; N1-((2S,3R)-3-hydroxy-4-(3-methoxybenzylamino)-1-phenylbutan-2-yl)-N3-methyl-N3-((5-methylfuran-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methyloxazol-2-yl)methyl)isophthalamide; N1-((2S,3R)-3-hydroxy-4-(3-methoxybenzylamino)-1-phenylbutan-2-yl)-N3-methyl-N3-(pyridin-2-ylmethyl)isophthalamide; N1-((2S,3R)-3-hydroxy-4-(3-methoxybenzylamino)-1-phenylbutan-2-yl)-N3-methyl-N3-(pyridin-3-ylmethyl)isophthalamide; N1-((2S,3R)-3-hydroxy-4-(3-methoxybenzylamino)-1-phenylbutan-2-yl)-N3-methyl-N3-((6-methylpyridin-2-yl)methyl)isophthalamide; N1-((2S,3R)-3-hydroxy-4-(3-methoxybenzylamino)-1-phenylbutan-2-yl)-N3-((4-methylpyrimidin-2-yl)methyl)isophthalamide; 5-fluoro-N1-((2S,3R)-3-hydroxy-4-(3-methoxybenzylamino)-1-phenylbutan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-N3,5-dimethyl-N3-((4-methylthiazol-2-yl)methyl)isophthalamide; N3-((2S,3R)-3-hydroxy-4-(3-methoxybenzylamino)-1-phenylbutan-2-yl)-N1,4-dimethyl-N1-((4-methylthiazol-2-yl)methyl)isophthalamide; N1-((2S,3R)-3-hydroxy-4-(3-methoxybenzylamino)-1-phenylbutan-2-yl)-N3,4-dimethyl-N3-((4-methylthiazol-2-yl)methyl)isophthalamide; 5-tert-butyl-N1-((2S,3R)-3-hydroxy-4-(3-methoxybenzylamino)-1-phenylbutan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-vinylisophthalamide; N1-cyclopropyl-N3-((2S,3R)-3-hydroxy-4-(3-methoxybenzylamino)-1-phenylbutan-2-yl)-5-methyl-N1-((4-methylthiazol-2-yl)methyl)isophthalamide; N1-((2S,3R)-3-hydroxy-4-((5-isopropylpyridin-3-yl)methylamino)-1-phenylbutan-2-yl)-N3,5-dimethyl-N3-((4-methylthiazol-2-yl)methyl)isophthalamide; N1-((2S,3R)-3-hydroxy-4-((2-isopropylpyridin-4-yl)methylamino)-1-phenylbutan-2-yl)-N3,5-dimethyl-N3-((4-methylthiazol-2-yl)methyl)isophthalamide; N1-cyclopropyl-N3-((2S,3R)-3-hydroxy-4-((5-isopropylpyridin-3-yl)methylamino)-1-phenylbutan-2-yl)-5-methyl-N1-((4-methylthiazol-2-yl)methyl)isophthalamide; 5-hydroxy-N1-((2S,3R)-3-hydroxy-4-(3-methoxybenzylamino)-1-phenylbutan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-5-methoxy-N3-methyl-N3-((4-methylthiazol-2-yl)methyl)isophthalamide; N1-((2S,3R)-3-hydroxy-1-phenyl-4-(3-(trifluoromethyl)benzylamino)butan-2-yl)-5-methoxy-N3-methyl-N3-((4-methylthiazol-2-yl)methyl)isophthalamide; N1-ethyl-N3-((2S,3R)-3-hydroxy-4-(3-methoxybenzylamino)-1-phenylbutan-2-yl)-5-methoxy-N1-((4-methylthiazol-2-yl)methyl)isophthalamide; N1-((2S,3R)-3-hydroxy-4-(3-methoxybenzylamino)-1-phenylbutan-2-yl)-5-methoxy-N3-((4-methylthiazol-2-yl)methyl)-N3-propylisophthalamide; N1-((2S,3R)-3-hydroxy-4-(3-methoxybenzylamino)-1-phenylbutan-2-yl)-N3-isopropyl-5-methoxy-N3-((4-methylthiazol-2-yl)methyl)isophthalamide; N1-((2S,3R)-3-hydroxy-4-(3-methoxybenzylamino)-1-phenylbutan-2-yl)-N3-(2-hydroxyethyl)-5-methoxy-N3-((4-methylthiazol-2-yl)methyl)isophthalamide; N1-((2S,3R)-3-hydroxy-4-((2-isopropylpyridin-4-yl)methylamino)-1-phenylbutan-2-yl)-5-methoxy-N3-methyl-N3-((4-methylthiazol-2-yl)methyl)isophthalamide; N1-((2S,3R)-3-hydroxy-4-((2-isopropylpyridin-4-yl)methylamino)-1-phenylbutan-2-yl)-5-methoxy-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-methoxy-N3-methyl-N3-((4-methylthiazol-2-yl)methyl)isophthalamide; N1-((2S,3R)-3-hydroxy-4-(3-methoxybenzylamino)-1-phenylbutan-2-yl)-5-methoxy-N3-((R)-1-phenylethyl)isophthalamide; N1-((2S,3R)-3-hydroxy-4-(3-methoxybenzylamino)-1-phenylbutan-2-yl)-5-(hydroxymethyl)-N3-methyl-N3-((4-methylthiazol-2-yl)methyl)isophthalamide; N1-((2S,3R)-3-hydroxy-4-(3-methoxybenzylamino)-1-phenylbutan-2-yl)-5-(methoxymethyl)-N3-methyl-N3-((4-methylthiazol-2-yl)methyl)isophthalamide; 3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzoic acid; methyl 3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzoate; N1-((2S,3R)-3-hydroxy-4-(3-methoxybenzylamino)-1-phenylbutan-2-yl)-N3-methyl-N3-((4-methylthiazol-2-yl)methyl)benzene-1,3,5-tricarboxamide; N1-((2S,3R)-3-hydroxy-4-(3-methoxybenzylamino)-1-phenylbutan-2-yl)-N3,N5-dimethyl-N3-((4-methylthiazol-2-yl)methyl)benzene-1,3,5-tricarboxamide; N1-((2S,3R)-3-hydroxy-4-(3-methoxybenzylamino)-1-phenylbutan-2-yl)-N3-(2-hydroxyethyl)-N5-methyl-N5-((4-methylthiazol-2-yl)methyl)benzene-1,3,5-tricarboxamide; (S)-methyl 3-hydroxy-2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzamido)propanoate; N1-((2S,3R)-3-hydroxy-4-(3-methoxybenzylamino)-1-phenylbutan-2-yl)-N3-((R)-1-phenylethyl)benzene-1,3,5-tricarboxamide; N1-((2S,3R)-3-hydroxy-4-(3-methoxybenzylamino)-1-phenylbutan-2-yl)-N3-methyl-N5-((R)-1-phenylethyl)benzene-1,3,5-tricarboxamide; 5-(4,5-dihydrooxazol-2-yl)-N1-((2S,3R)-3-hydroxy-4-(3-methoxybenzylamino)-1-phenylbutan-2-yl)-N3-methyl-N3-((4-methylthiazol-2-yl)methyl)isophthalamide; (S)-methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)-4,5-dihydrooxazole-4-carboxylate; N1-((2S,3R)-3-hydroxy-4-(3-methoxybenzylamino)-1-phenylbutan-2-yl)-N3-methyl-N3-((4-methylthiazol-2-yl)methyl)-5-(oxazol-2-yl)isophthalamide; methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)oxazole-4-carboxylate; N3-((2S,3R)-3-hydroxy-4-(3-methoxybenzylamino)-1-phenylbutan-2-yl)-N5-methyl-N5-((4-methylthiazol-2-yl)methyl)biphenyl-3,5-dicarboxamide; 5-(azidomethyl)-N1-((2S,3R)-3-hydroxy-4-(3-methoxybenzylamino)-1-phenylbutan-2-yl)-N3-methyl-N3-((4-methylthiazol-2-yl)methyl)isophthalamide; 5-amino-N1-((2S,3R)-3-hydroxy-4-(3-methoxybenzylamino)-1-phenylbutan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-N3-methyl-5-(methylamino)-N3-((4-methylthiazol-2-yl)methyl)isophthalamide; 5-(dimethylamino)-N1-((2S,3R)-3-hydroxy-4-(3-methoxybenzylamino)-1-phenylbutan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-5-(hydroxyamino)-N3-methyl-N3-((4-methylthiazol-2-yl)methyl)isophthalamide; N1-((2S,3R)-3-hydroxy-4-(3-methoxybenzylamino)-1-phenylbutan-2-yl)-5-(hydroxyamino)-N3-((R)-1-phenylethyl)isophthalamide; 3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl methanesulfonate; 3-((2S,3R)-3-hydroxy-4-((5-isopropylpyridin-3-yl)methylamino)-1-phenylbutan-2-ylcarbamoyl)-5-(methyl((4-methylthiazol-2-yl)methyl)carbamoyl)phenyl methanesulfonate; 3-((2S,3R)-3-hydroxy-4-(3-methoxybenzylamino)-1-phenylbutan-2-ylcarbamoyl)-5-((R)-1-phenylethylcarbamoyl)phenyl methanesulfonate; N1-((2S,3R)-3-hydroxy-1-phenyl-4-((S)-1-phenylethylamino)butan-2-yl)-N3-((S)-1-phenylethyl)isophthalamide; N1-((2S,3R)-3-hydroxy-1-phenyl-4-((S)-1-phenylethylamino)butan-2-yl)-N3-((R)-1-p-tolylethyl)isophthalamide; N1-((2S,3R)-3-hydroxy-1-phenyl-4-((S)-1-phenylethylamino)butan-2-yl)-N3-((R)-1-(4-methoxyphenyl)ethyl)isophthalamide; N1-((2S,3R)-3-hydroxy-1-phenyl-4-((S)-1-phenylethylamino)butan-2-yl)-N3-((R)-1-(3-methoxyphenyl)ethyl)isophthalamide; N1-((2S,3R)-3-hydroxy-1-phenyl-4-((S)-1-phenylethylamino)butan-2-yl)-N3-(1-o-tolylethyl)isophthalamide; N1-((2S,3R)-3-hydroxy-4-(methyl((S)-1-phenylethyl)amino)-1-phenylbutan-2-yl)-N3-(1-(3-methylthiophen-2-yl)ethyl)isophthalamide; N1-((2S,3R)-3-hydroxy-1-phenyl-4-((R)-1-p-tolylethylamino)butan-2-yl)-N3-((R)-1-p-tolylethyl)isophthalamide; N1-((2S,3R)-3-hydroxy-1-phenyl-4-((R)-1-p-tolylethylamino)butan-2-yl)-N3-((R)-1-(4-methoxyphenyl)ethyl)isophthalamide; N1-((2S,3R)-3-hydroxy-4-((R)-1-(4-methoxyphenyl)ethylamino)-1-phenylbutan-2-yl)-N3-((R)-1-p-tolylethyl)isophthalamide; N1-((2S,3R)-3-hydroxy-4-((R)-1-(4-methoxyphenyl)ethylamino)-1-phenylbutan-2-yl)-N3-((R)-1-(4-methoxyphenyl)ethyl)isophthalamide; N1-((2S,3R)-3-hydroxy-1-phenyl-4-((S)-1-phenylethylamino)butan-2-yl)-5-methyl-N3-((S)-1-phenylethyl)isophthalamide; N1-((2S,3R)-3-hydroxy-1-phenyl-4-((S)-1-phenylethylamino)butan-2-yl)-5-methyl-N3-(1-(5-methylfuran-2-yl)ethyl)isophthalamide; N1-((2S,3R)-3-hydroxy-1-phenyl-4-((S)-1-phenylethylamino)butan-2-yl)-5-methyl-N3-(1-o-tolylethyl)isophthalamide; N1-((2S,3R)-3-hydroxy-4-(methyl((S)-1-phenylethyl)amino)-1-phenylbutan-2-yl)-5-methyl-N3-((R)-1-p-tolylethyl)isophthalamide; N1-((2S,3R)-3-hydroxy-4-(methyl((S)-1-phenylethyl)amino)-1-phenylbutan-2-yl)-N3-((R)-1-(4-methoxyphenyl)ethyl)-5-methylisophthalamide; N1-((2S,3R)-3-hydroxy-4-(methyl((S)-1-phenylethyl)amino)-1-phenylbutan-2-yl)-N3-((R)-1-(3-methoxyphenyl)ethyl)-5-methylisophthalamide; N1-((2S,3R)-3-hydroxy-4-(methyl((S)-1-phenylethyl)amino)-1-phenylbutan-2-yl)-5-methyl-N3-(1-(5-methylthiophen-2-yl)ethyl)isophthalamide; N1-((2S,3R)-3-hydroxy-1-phenyl-4-((R)-1-p-tolylethylamino)butan-2-yl)-5-methyl-N3-((R)-1-p-tolylethyl)isophthalamide; N1-((2S,3R)-3-hydroxy-1-phenyl-4-((R)-1-p-tolylethylamino)butan-2-yl)-5-methyl-N3-(1-o-tolylethyl)isophthalamide; N1-((2S,3R)-3-hydroxy-4-((R)-1-(3-methoxyphenyl)ethylamino)-1-phenylbutan-2-yl)-5-methyl-N3-(1-(pyrazin-2-yl)ethyl)isophthalamide; N1-((2S,3R)-3-hydroxy-4-(methyl((S)-1-phenylethyl)amino)-1-phenylbutan-2-yl)-N3-((S)-1-phenylethyl)isophthalamide; N1-(1-(furan-2-yl)ethyl)-N3-((2S,3R)-3-hydroxy-1-phenyl-4-((S)-1-phenylethylamino)butan-2-yl)isophthalamide; N1-((2S,3R)-3-hydroxy-4-(methyl((S)-1-phenylethyl)amino)-1-phenylbutan-2-yl)-N3-((R)-1-p-tolylethyl)isophthalamide; N1-((2S,3R)-3-hydroxy-4-(methyl((S)-1-phenylethyl)amino)-1-phenylbutan-2-yl)-N3-((R)-1-(4-methoxyphenyl)ethyl)isophthalamide; N1-(1-(furan-2-yl)ethyl)-N3-((2S,3R)-3-hydroxy-4-(methyl((S)-1-phenylethyl)amino)-1-phenylbutan-2-yl)isophthalamide; N1-((2S,3R)-3-hydroxy-1-phenyl-4-((R)-1-p-tolylethylamino)butan-2-yl)-N3-(1-o-tolylethyl)isophthalamide; N1-((2S,3R)-3-hydroxy-4-((R)-1-(4-methoxyphenyl)ethylamino)-1-phenylbutan-2-yl)-N3-((S)-1-phenylethyl)isophthalamide; N1-((2S,3R)-3-hydroxy-4-((R)-1-(4-methoxyphenyl)ethylamino)-1-phenylbutan-2-yl)-N3-((R)-1-(3-methoxyphenyl)ethyl)isophthalamide; N1-(1-(2,3-dihydrofuran-2-yl)ethyl)-N3-((2S,3R)-3-hydroxy-4-((R)-1-(4-methoxyphenyl)ethylamino)-1-phenylbutan-2-yl)isophthalamide; N1-((2S,3R)-3-hydroxy-4-((R)-1-(4-methoxyphenyl)ethylamino)-1-phenylbutan-2-yl)-N3-(1-(5-methyl-2,3-dihydrofuran-2-yl)ethyl)isophthalamide; N1-((2S,3R)-3-hydroxy-4-((R)-1-(4-methoxyphenyl)ethylamino)-1-phenylbutan-2-yl)-N3-(1-o-tolylethyl)isophthalamide; N1-((2S,3R)-3-hydroxy-4-((R)-1-(4-methoxyphenyl)ethylamino)-1-phenylbutan-2-yl)-N3-(1-(pyrazin-2-yl)ethyl)isophthalamide; N1-((2S,3R)-3-hydroxy-4-((R)-1-(4-methoxyphenyl)ethylamino)-1-phenylbutan-2-yl)-N3-(1-(3-methylthiophen-2-yl)ethyl)isophthalamide; N1-((2S,3R)-3-hydroxy-4-((R)-1-(3-methoxyphenyl)ethylamino)-1-phenylbutan-2-yl)-N3-((S)-1-phenylethyl)isophthalamide; N1-((2S,3R)-3-hydroxy-4-((R)-1-(3-methoxyphenyl)ethylamino)-1-phenylbutan-2-yl)-N3-((R)-1-(3-methoxyphenyl)ethyl)isophthalamide; N1-(1-(furan-2-yl)ethyl)-N3-((2S,3R)-3-hydroxy-4-((R)-1-(3-methoxyphenyl)ethylamino)-1-phenylbutan-2-yl)isophthalamide; N1-((2S,3R)-3-hydroxy-4-((R)-1-(3-methoxyphenyl)ethylamino)-1-phenylbutan-2-yl)-N3-(1-o-tolylethyl)isophthalamide; N1-((2S,3R)-3-hydroxy-1-phenyl-4-((S)-1-phenylethylamino)butan-2-yl)-N3-((R)-1-(3-methoxyphenyl)ethyl)-5-methylisophthalamide; N1-((2S,3R)-3-hydroxy-1-phenyl-4-((S)-1-phenylethylamino)butan-2-yl)-5-methyl-N3-(1-(pyrazin-2-yl)ethyl)isophthalamide; N1-((2S,3R)-3-hydroxy-4-(methyl((S)-1-phenylethyl)amino)-1-phenylbutan-2-yl)-5-methyl-N3-(1-(pyrazin-2-yl)ethyl)isophthalamide; N1-((2S,3R)-3-hydroxy-1-phenyl-4-((R)-1-p-tolylethylamino)butan-2-yl)-N3,5-dimethyl-N3-((S)-1-phenylethyl)isophthalamide; N1-((2S,3R)-3-hydroxy-1-phenyl-4-((R)-1-p-tolylethylamino)butan-2-yl)-N3-((R)-1-(4-methoxyphenyl)ethyl)-5-methylisophthalamide; N1-((2S,3R)-3-hydroxy-1-phenyl-4-((R)-1-p-tolylethylamino)butan-2-yl)-N3-((R)-1-(3-methoxyphenyl)ethyl)-5-methylisophthalamide; N1-(1-(furan-2-yl)ethyl)-N3-((2S,3R)-3-hydroxy-1-phenyl-4-((R)-1-p-tolylethylamino)butan-2-yl)-5-methylisophthalamide; N1-((2S,3R)-3-hydroxy-1-phenyl-4-((R)-1-p-tolylethylamino)butan-2-yl)-5-methyl-N3-(1-(5-methylfuran-2-yl)ethyl)isophthalamide; N1-((2S,3R)-3-hydroxy-1-phenyl-4-((R)-1-p-tolylethylamino)butan-2-yl)-5-methyl-N3-(1-(pyrazin-2-yl)ethyl)isophthalamide; N1-((2S,3R)-3-hydroxy-4-((R)-1-(4-methoxyphenyl)ethylamino)-1-phenylbutan-2-yl)-5-methyl-N3-((S)-1-phenylethyl)isophthalamide; N1-((2S,3R)-3-hydroxy-4-((R)-1-(4-methoxyphenyl)ethylamino)-1-phenylbutan-2-yl)-5-methyl-N3-((R)-1-p-tolylethyl)isophthalamide; N1-((2S,3R)-3-hydroxy-4-((R)-1-(4-methoxyphenyl)ethylamino)-1-phenylbutan-2-yl)-N3-((R)-1-(4-methoxyphenyl)ethyl)-5-methylisophthalamide; N1-((2S,3R)-3-hydroxy-4-((R)-1-(4-methoxyphenyl)ethylamino)-1-phenylbutan-2-yl)-5-methyl-N3-(1-(5-methylfuran-2-yl)ethyl)isophthalamide; N1-((2S,3R)-3-hydroxy-4-((R)-1-(4-methoxyphenyl)ethylamino)-1-phenylbutan-2-yl)-5-methyl-N3-(1-(pyrazin-2-yl)ethyl)isophthalamide; N1-((2S,3R)-3-hydroxy-4-((R)-1-(4-methoxyphenyl)ethylamino)-1-phenylbutan-2-yl)-5-methyl-N3-(1-(5-methylthiophen-2-yl)ethyl)isophthalamide; N1-((2S,3R)-3-hydroxy-4-((R)-1-(4-methoxyphenyl)ethylamino)-1-phenylbutan-2-yl)-5-methyl-N3-(1-(3-methylthiophen-2-yl)ethyl)isophthalamide; N1-((2S,3R)-3-hydroxy-4-((R)-1-(3-methoxyphenyl)ethylamino)-1-phenylbutan-2-yl)-5-methyl-N3-((S)-1-phenylethyl)isophthalamide; N1-((2S,3R)-3-hydroxy-4-((R)-1-(3-methoxyphenyl)ethylamino)-1-phenylbutan-2-yl)-5-methyl-N3-((R)-1-p-tolylethyl)isophthalamide; N1-((2S,3R)-3-hydroxy-4-((R)-1-(3-methoxyphenyl)ethylamino)-1-phenylbutan-2-yl)-N3-((R)-1-(4-methoxyphenyl)ethyl)-5-methylisophthalamide; N1-((2S,3R)-3-hydroxy-4-((R)-1-(3-methoxyphenyl)ethylamino)-1-phenylbutan-2-yl)-N3-((R)-1-(3-methoxyphenyl)ethyl)-5-methylisophthalamide; N1-(1-(furan-2-yl)ethyl)-N3-((2S,3R)-3-hydroxy-4-((R)-1-(3-methoxyphenyl)ethylamino)-1-phenylbutan-2-yl)-5-methylisophthalamide; N1-((2S,3R)-3-hydroxy-4-((R)-1-(3-methoxyphenyl)ethylamino)-1-phenylbutan-2-yl)-5-methyl-N3-(1-(5-methylfuran-2-yl)ethyl)isophthalamide; N1-((2S,3R)-3-hydroxy-4-((R)-1-(3-methoxyphenyl)ethylamino)-1-phenylbutan-2-yl)-5-methyl-N3-(1-o-tolylethyl)isophthalamide; N1-((2S,3R)-3-hydroxy-4-((R)-1-(3-methoxyphenyl)ethylamino)-1-phenylbutan-2-yl)-5-methyl-N3-(1-(5-methylthiophen-2-yl)ethyl)isophthalamide; N1-((2S,3R)-3-hydroxy-4-((R)-1-(3-methoxyphenyl)ethylamino)-1-phenylbutan-2-yl)-5-methyl-N3-(1-(3-methylthiophen-2-yl)ethyl)isophthalamide; N1-((2S,3R)-3-hydroxy-4-((R)-1-(3-methoxyphenyl)ethylamino)-1-phenylbutan-2-yl)-N3-(1-(3-methoxyphenyl)ethyl)-N3,5-dimethylisophthalamide; N1-((2S,3R)-4-(1-(furan-2-yl)ethylamino)-3-hydroxy-1-phenylbutan-2-yl)-5-methyl-N3-((R)-1-p-tolylethyl)isophthalamide; N1-((2S,3R)-3-hydroxy-1-phenyl-4-((R)-1-p-tolylethylamino)butan-2-yl)-N3-(1-(pyrazin-2-yl)ethyl)isophthalamide; N1-((2S,3R)-4-(1-(furan-2-yl)ethylamino)-3-hydroxy-1-phenylbutan-2-yl)-N3-((S)-1-phenylethyl)isophthalamide; N1-((2S,3R)-4-(1-(furan-2-yl)ethylamino)-3-hydroxy-1-phenylbutan-2-yl)-N3-((R)-1-p-tolylethyl)isophthalamide; N1-((2S,3R)-4-(1-(furan-2-yl)ethylamino)-3-hydroxy-1-phenylbutan-2-yl)-N3-((R)-1-(4-methoxyphenyl)ethyl)isophthalamide; N1-((2S,3R)-4-(1-(furan-2-yl)ethylamino)-3-hydroxy-1-phenylbutan-2-yl)-N3-((R)-1-(3-methoxyphenyl)ethyl)isophthalamide; N1-((2S,3R)-4-(1-(furan-2-yl)ethylamino)-3-hydroxy-1-phenylbutan-2-yl)-N3-(1-(3-methylthiophen-2-yl)ethyl)isophthalamide; N1-(1-(furan-2-yl)ethyl)-N3-((2S,3R)-3-hydroxy-1-phenyl-4-((S)-1-phenylethylamino)butan-2-yl)-5-methylisophthalamide; N1-((2S,3R)-3-hydroxy-4-(methyl((S)-1-phenylethyl)amino)-1-phenylbutan-2-yl)-5-methyl-N3-(1-(3-methylthiophen-2-yl)ethyl)isophthalamide; N1-((2S,3R)-3-hydroxy-1-phenyl-4-((R)-1-p-tolylethylamino)butan-2-yl)-5-methyl-N3-((S)-1-phenylethyl)isophthalamide; N1-((2S,3R)-3-hydroxy-4-((R)-1-(4-methoxyphenyl)ethylamino)-1-phenylbutan-2-yl)-N3-((R)-1-(3-methoxyphenyl)ethyl)-5-methylisophthalamide; N1-((2S,3R)-3-hydroxy-4-((R)-1-(4-methoxyphenyl)ethylamino)-1-phenylbutan-2-yl)-5-methyl-N3-(1-o-tolylethyl)isophthalamide; N1-((2S,3R)-4-(1-(furan-2-yl)ethylamino)-3-hydroxy-1-phenylbutan-2-yl)-5-methyl-N3-((S)-1-phenylethyl)isophthalamide; N1-((2S,3R)-4-(1-(furan-2-yl)ethylamino)-3-hydroxy-1-phenylbutan-2-yl)-N3-((R)-1-(3-methoxyphenyl)ethyl)-5-methylisophthalamide; N1-((2S,3R)-4-(1-(furan-2-yl)ethylamino)-3-hydroxy-1-phenylbutan-2-yl)-5-methyl-N3-(1-(3-methylthiophen-2-yl)ethyl)isophthalamide; N1-((2S,3R)-3-hydroxy-4-(methyl((S)-1-phenylethyl)amino)-1-phenylbutan-2-yl)-N3-(1-o-tolylethyl)isophthalamide; N1-((2S,3R)-3-hydroxy-4-((R)-1-(3-methoxyphenyl)ethylamino)-1-phenylbutan-2-yl)-N3-(1-(5-methylthiophen-2-yl)ethyl)isophthalamide; N1-((2S,3R)-3-hydroxy-4-((R)-1-(3-methoxyphenyl)ethylamino)-1-phenylbutan-2-yl)-N3-(1-(4-methoxyphenyl)ethyl)-N3-methylisophthalamide; N1-((2S,3R)-3-hydroxy-1-phenyl-4-((S)-1-phenylethylamino)butan-2-yl)-5-methyl-N3-((S)-1-p-tolylethyl)isophthalamide; N1-(1-(furan-2-yl)ethyl)-N3-((2S,3R)-3-hydroxy-4-((R)-1-(4-methoxyphenyl)ethylamino)-1-phenylbutan-2-yl)-5-methylisophthalamide; N1-((2S,3R)-4-(1-(furan-2-yl)ethylamino)-3-hydroxy-1-phenylbutan-2-yl)-5-methyl-N3-(1-(5-methylthiophen-2-yl)ethyl)isophthalamide; N1-((2S,3R)-3-hydroxy-4-(methyl((S)-1-phenylethyl)amino)-1-phenylbutan-2-yl)-5-methyl-N3-(1-(5-methylfuran-2-yl)ethyl)isophthalamide; N1-(1-(furan-2-yl)ethyl)-N3-((2S,3R)-3-hydroxy-1-phenyl-4-((R)-1-p-tolylethylamino)butan-2-yl)isophthalamide; N1-((2S,3R)-3-hydroxy-4-((R)-1-(3-methoxyphenyl)ethylamino)-1-phenylbutan-2-yl)-N3-((R)-1-(4-methoxyphenyl)ethyl)isophthalamide; N1-((2S,3R)-3-hydroxy-1-phenyl-4-((R)-1-p-tolylethylamino)butan-2-yl)-N3-(1-(4-methoxyphenyl)ethyl)-N3,5-dimethylisophthalamide; N1-((2S,3R)-4-(1-(furan-2-yl)ethylamino)-3-hydroxy-1-phenylbutan-2-yl)-5-methyl-N3-(1-o-tolylethyl)isophthalamide; N1-(1-(furan-2-yl)ethyl)-N3-((2S,3R)-4-(1-(furan-2-yl)ethylamino)-3-hydroxy-1-phenylbutan-2-yl)-5-methylisophthalamide; N1-((2S,3R)-3-hydroxy-1-phenyl-4-((R)-1-p-tolylethylamino)butan-2-yl)-5-methyl-N3-(1-(3-methylthiophen-2-yl)ethyl)isophthalamide; N1-((2S,3R)-3-hydroxy-1-phenyl-4-((R)-1-p-tolylethylamino)butan-2-yl)-N3-(1-(4-methoxyphenyl)ethyl)-N3-methylisophthalamide; N1-((2S,3R)-3-hydroxy-1-phenyl-4-((R)-1-p-tolylethylamino)butan-2-yl)-N3-(1-(5-methylthiophen-2-yl)ethyl)isophthalamide; N1-((2S,3R)-3-hydroxy-1-phenyl-4-((R)-1-p-tolylethylamino)butan-2-yl)-N3-(1-(3-methoxyphenyl)ethyl)-N3-methylisophthalamide; N1-((2S,3R)-4-(1-(furan-2-yl)ethylamino)-3-hydroxy-1-phenylbutan-2-yl)-N3-(1-(5-methylfuran-2-yl)ethyl)isophthalamide; N1-((2S,3R)-3-hydroxy-4-(methyl((S)-1-phenylethyl)amino)-1-phenylbutan-2-yl)-5-methyl-N3-(1-o-tolylethyl)isophthalamide; N1-((2S,3R)-3-hydroxy-1-phenyl-4-((S)-1-phenylethylamino)butan-2-yl)-N3-(1-(5-methylthiophen-2-yl)ethyl)isophthalamide; N1-((2S,3R)-3-hydroxy-4-(methyl((S)-1-phenylethyl)amino)-1-phenylbutan-2-yl)-N3-(1-(5-methylfuran-2-yl)ethyl)isophthalamide; N1-((2S,3R)-3-hydroxy-1-phenyl-4-((R)-1-p-tolylethylamino)butan-2-yl)-N3-((R)-1-(3-methoxyphenyl)ethyl)isophthalamide; N1-((2S,3R)-3-hydroxy-4-((R)-1-(4-methoxyphenyl)ethylamino)-1-phenylbutan-2-yl)-N3-(1-(5-methylthiophen-2-yl)ethyl)isophthalamide; N1-((2S,3R)-3-hydroxy-4-((R)-1-(3-methoxyphenyl)ethylamino)-1-phenylbutan-2-yl)-N3-methyl-N3-((S)-1-phenylethyl)isophthalamide; N1-((2S,3R)-3-hydroxy-4-((R)-1-(3-methoxyphenyl)ethylamino)-1-phenylbutan-2-yl)-N3-(1-(3-methylthiophen-2-yl)ethyl)isophthalamide; N1-((2S,3R)-3-hydroxy-4-(methyl((S)-1-phenylethyl)amino)-1-phenylbutan-2-yl)-N3,5-dimethyl-N3-((S)-1-phenylethyl)isophthalamide; N1-((2S,3R)-3-hydroxy-4-(methyl((S)-1-phenylethyl)amino)-1-phenylbutan-2-yl)-N3-(1-(4-methoxyphenyl)ethyl)-N3,5-dimethylisophthalamide; N1-((2S,3R)-3-hydroxy-1-phenyl-4-((R)-1-p-tolylethylamino)butan-2-yl)-5-methyl-N3-(1-(5-methylthiophen-2-yl)ethyl)isophthalamide; N1-((2S,3R)-3-hydroxy-4-((R)-1-(4-methoxyphenyl)ethylamino)-1-phenylbutan-2-yl)-N3,5-dimethyl-N3-((S)-1-phenylethyl)isophthalamide; N1-((2S,3R)-3-hydroxy-4-((R)-1-(4-methoxyphenyl)ethylamino)-1-phenylbutan-2-yl)-N3-(1-(3-methoxyphenyl)ethyl)-N3,5-dimethylisophthalamide; N1-((2S,3R)-3-hydroxy-4-((R)-1-(3-methoxyphenyl)ethylamino)-1-phenylbutan-2-yl)-N3,5-dimethyl-N3-((S)-1-phenylethyl)isophthalamide; N1-((2S,3R)-4-(1-(furan-2-yl)ethylamino)-3-hydroxy-1-phenylbutan-2-yl)-5-methyl-N3-(1-(pyridin-2-yl)ethyl)isophthalamide; N1-((2S,3R)-3-hydroxy-4-((R)-1-(4-methoxyphenyl)ethylamino)-1-phenylbutan-2-yl)-5-methyl-N3-(1-(pyridin-2-yl)ethyl)isophthalamide; N1-((2S,3R)-3-hydroxy-1-phenyl-4-((S)-1-phenylethylamino)butan-2-yl)-N3-(1-(5-methylfuran-2-yl)ethyl)isophthalamide; N1-((2S,3R)-3-hydroxy-4-(methyl((S)-1-phenylethyl)amino)-1-phenylbutan-2-yl)-N3-(1-(pyrazin-2-yl)ethyl)isophthalamide; N1-((2S,3R)-3-hydroxy-4-((R)-1-(4-methoxyphenyl)ethylamino)-1-phenylbutan-2-yl)-N3-methyl-N3-((S)-1-phenylethyl)isophthalamide; N1-((2S,3R)-3-hydroxy-4-(methyl((S)-1-phenylethyl)amino)-1-phenylbutan-2-yl)-N3-(1-(5-methylthiophen-2-yl)ethyl)isophthalamide; N1-((2S,3R)-3-hydroxy-1-phenyl-4-((R)-1-p-tolylethylamino)butan-2-yl)-N3-((S)-1-phenylethyl)isophthalamide; N1-((2S,3R)-3-hydroxy-4-(methyl((S)-1-phenylethyl)amino)-1-phenylbutan-2-yl)-N3-((R)-1-(3-methoxyphenyl)ethyl)isophthalamide; N1-((2S,3R)-3-hydroxy-1-phenyl-4-((R)-1-p-tolylethylamino)butan-2-yl)-5-methyl-N3-(1-(pyridin-2-yl)ethyl)isophthalamide; N1-((2S,3R)-3-hydroxy-4-((R)-1-(3-methoxyphenyl)ethylamino)-1-phenylbutan-2-yl)-N3-((R)-1-p-tolylethyl)isophthalamide; N1-((2S,3R)-3-hydroxy-1-phenyl-4-((S)-1-phenylethylamino)butan-2-yl)-5-methyl-N3-(1-(pyridin-2-yl)ethyl)isophthalamide; N1-((2S,3R)-3-hydroxy-4-((R)-1-(4-methoxyphenyl)ethylamino)-1-phenylbutan-2-yl)-N3-(1-(3-methoxyphenyl)ethyl)-N3-methylisophthalamide; N1-((2S,3R)-3-hydroxy-1-phenyl-4-((S)-1-phenylethylamino)butan-2-yl)-N3-(1-(3-methoxyphenyl)ethyl)-N3,5-dimethylisophthalamide; N1-((2S,3R)-3-hydroxy-4-(methyl((S)-1-phenylethyl)amino)-1-phenylbutan-2-yl)-5-methyl-N3-((S)-1-phenylethyl)isophthalamide; N1-((2S,3R)-4-(3-(diethylamino)benzylamino)-3-hydroxy-1-phenylbutan-2-yl)-N3-methyl-N3-((4-methylthiazol-2-yl)methyl)isophthalamide; 5-(dimethylamino)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-4-(3-(benzylamino)benzylamino)-3-hydroxy-1-phenylbutan-2-yl)-N3-methyl-N3-((4-methylthiazol-2-yl)methyl)isophthalamide; N1-((2S,3R)-3-hydroxy-4-(3-methoxyphenylamino)-1-phenylbutan-2-yl)-N3-methyl-N3-((4-methylthiazol-2-yl)methyl)isophthalamide; N1-((2S,3R)-3-hydroxy-4-(3-(methylamino)benzylamino)-1-phenylbutan-2-yl)-N3-methyl-N3-((4-methylthiazol-2-yl)methyl)isophthalamide; 5-(diethylamino)-N1-((2S,3R)-3-hydroxy-4-(3-methoxybenzylamino)-1-phenylbutan-2-yl)-N3-methyl-N3-((4-methylthiazol-2-yl)methyl)isophthalamide; 5-(diethylamino)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 3-(((2R,3S)-3-(3-(dimethylamino)-5-(methyl((4-methylthiazol-2-yl)methyl)carbamoyl)benzamido)-2-hydroxy-4-phenylbutylamino)methyl)-5-isopropylphenyl methanesulfonate; N1-((2S,3R)-3-hydroxy-4-(3-methoxybenzylamino)-1-phenylbutan-2-yl)-N3-methyl-N3-((4-methylthiazol-2-yl)methyl)-5-(pyrrolidin-1-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyrrolidin-1-yl)isophthalamide; N1-((2S,3R)-4-(3,5-bis(dimethylamino)benzylamino)-3-hydroxy-1-phenylbutan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-N3-((4-methylthiazol-2-yl)methyl)-5-(pyrrolidin-1-yl)isophthalamide; N1-((2S,3R)-4-(3-aminobenzylamino)-3-hydroxy-1-phenylbutan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-(2-oxopyrrolidin-1-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(2-oxopyrrolidin-1-yl)isophthalamide; N1-((2S,3R)-3-hydroxy-4-(3-methoxybenzylamino)-1-phenylbutan-2-yl)-N3-((4-methylthiazol-2-yl)methyl)-5-(2-oxopyrrolidin-1-yl)isophthalamide; N1-((2S,3R)-4-((2-amino-6-methoxypyrimidin-4-yl)methylamino)-3-hydroxy-1-phenylbutan-2-yl)-N3-methyl-N3-((4-methylthiazol-2-yl)methyl)isophthalamide; N1-((2S,3R)-4-((2-(dimethylamino)-6-methoxypyrimidin-4-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-(3-methoxy-5-nitrobenzylamino)-1-phenylbutan-2-yl)-N3-methyl-N3-((4-methylthiazol-2-yl)methyl)isophthalamide; N1-((2S,3R)-4-(3-(dimethylamino)-5-methoxybenzylamino)-3-hydroxy-1-phenylbutan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-(piperidin-1-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(piperidin-1-yl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(2-oxopyrrolidin-1-yl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-(1H-pyrrol-1-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrrol-1-yl)isophthalamide; N1-((2S,3R)-3-hydroxy-4-(1-(5-isopropylpyridin-3-yl)ethylamino)-1-phenylbutan-2-yl)-N3-methyl-N3-((4-methylthiazol-2-yl)methyl)isophthalamide; N1-((2S,3R)-3-hydroxy-4-((S)-1-(5-isopropylpyridin-3-yl)ethylamino)-1-phenylbutan-2-yl)-N3-methyl-N3-((4-methylthiazol-2-yl)methyl)isophthalamide; N1-((2S,3R)-3-hydroxy-4-((R)-1-(5-isopropylpyridin-3-yl)ethylamino)-1-phenylbutan-2-yl)-N3-methyl-N3-((4-methylthiazol-2-yl)methyl)isophthalamide; N1-((2S,3R)-3-hydroxy-4-(3-isopropyl-5-(N-methylmethylsulfonamido)benzylamino)-1-phenylbutan-2-yl)-N3-methyl-N3-((4-methylthiazol-2-yl)methyl)isophthalamide; N1-((2S,3R)-3-hydroxy-4-(3-isopropyl-5-(methylsulfonyl)benzylamino)-1-phenylbutan-2-yl)-N3-methyl-N3-((4-methylthiazol-2-yl)methyl)isophthalamide; N1-((2S,3R)-3-hydroxy-4-((5-isopropyl-2-methylpyridin-3-yl)methylamino)-1-phenylbutan-2-yl)-N3-methyl-N3-((4-methylthiazol-2-yl)methyl)isophthalamide; N1-((2S,3R)-3-hydroxy-4-(3-hydroxy-5-(prop-1-en-2-yl)benzylamino)-1-phenylbutan-2-yl)-N3-methyl-N3-((4-methylthiazol-2-yl)methyl)isophthalamide; 3-(((2R,3S)-2-hydroxy-3-(3-(methyl((4-methylthiazol-2-yl)methyl)carbamoyl)benzamido)-4-phenylbutylamino)methyl)-5-isopropylphenyl methanesulfonate; N1-((2S,3R)-3-hydroxy-4-(3-isopropyl-5-(methylsulfonamidomethyl)benzylamino)-1-phenylbutan-2-yl)-N3-methyl-N3-((4-methylthiazol-2-yl)methyl)isophthalamide; N1-((2S,3R)-4-(3-(ethylsulfonyl)-5-isopropylbenzylamino)-3-hydroxy-1-phenylbutan-2-yl)-N3-methyl-N3-((4-methylthiazol-2-yl)methyl)isophthalamide; N1-((2S,3R)-4-(3-acetamido-5-isopropylbenzylamino)-3-hydroxy-1-phenylbutan-2-yl)-N3-methyl-N3-((4-methylthiazol-2-yl)methyl)isophthalamide; 3-(((2R,3S)-2-hydroxy-3-(3-(methyl((4-methylthiazol-2-yl)methyl)carbamoyl)benzamido)-4-phenylbutylamino)methyl)-5-isopropylphenyl dimethylcarbamate; methyl 3-(((2R,3S)-2-hydroxy-3-(3-(methyl((4-methylthiazol-2-yl)methyl)carbamoyl)benzamido)-4-phenylbutylamino)methyl)-5-isopropylphenylcarbamate; 3-(((2R,3S)-3-(3-(dimethylamino)-5-(methyl((4-methylthiazol-2-yl)methyl)carbamoyl)benzamido)-2-hydroxy-4-phenylbutylamino)methyl)-5-isopropylphenyl dimethylcarbamate; N1-((2S,3R)-4-(3-tert-butyl-5-hydroxybenzylamino)-3-hydroxy-1-phenylbutan-2-yl)-N3-methyl-N3-((4-methylthiazol-2-yl)methyl)isophthalamide; N1-((2S,3R)-3-hydroxy-4-(3-isopropyl-5-(methylsulfonylmethyl)benzylamino)-1-phenylbutan-2-yl)-N3-methyl-N3-((4-methylthiazol-2-yl)methyl)isophthalamide; N1-((2S,3R)-3-hydroxy-4-(3-isopropyl-5-(isopropylsulfonyl)benzylamino)-1-phenylbutan-2-yl)-N3-methyl-N3-((4-methylthiazol-2-yl)methyl)isophthalamide; 3-(((2R,3S)-2-hydroxy-3-(3-(methyl((4-methylthiazol-2-yl)methyl)carbamoyl)benzamido)-4-phenylbutylamino)methyl)-5-isopropylphenyl morpholine-4-carboxylate; N1-((2S,3S)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2R,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-4-((2-amino-6-isopropylpyrimidin-4-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((6-isopropyl-2-(methylamino)pyrimidin-4-yl)methylamino)-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-2-yl)isophthalamide; N1-((2S,3R)-4-((2-amino-6-isopropylpyrimidin-4-yl)methylamino)-3-hydroxy-1-phenylbutan-2-yl)-N3-methyl-N3-((4-methylthiazol-2-yl)methyl)-5-(oxazol-2-yl)isophthalamide; N1-cyclopropyl-N3-((2S,3R)-3-hydroxy-4-((5-isopropylpyridin-3-yl)methylamino)-1-phenylbutan-2-yl)-N1-((4-methylthiazol-2-yl)methyl)-5-(oxazol-2-yl)isophthalamide; N1-((2S,3R)-3-hydroxy-4-((5-isopropylpyridin-3-yl)methylamino)-1-phenylbutan-2-yl)-5-(hydroxymethyl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-((4-methylthiazol-2-yl)methyl)-5-(oxazol-2-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-5-yl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-5-yl)isophthalamide; 5-acetyl-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-4-(3-(difluoromethyl)benzylamino)-3-hydroxy-1-phenylbutan-2-yl)-5-(N-methylmethylsulfonamido)-N3-((R)-1-phenylethyl)isophthalamide; N1-((2S,3R)-4-(3-(difluoromethyl)benzylamino)-3-hydroxy-1-phenylbutan-2-yl)-N3-methyl-N3-((4-methylthiazol-2-yl)methyl)isophthalamide; N1-((2S,3R)-4-(3-(1,1-difluoroethyl)benzylamino)-3-hydroxy-1-phenylbutan-2-yl)-N3-methyl-N3-((4-methylthiazol-2-yl)methyl)isophthalamide; N1-((2S,3R)-4-(bis((1-methyl-1H-pyrazol-4-yl)methyl)amino)-3-hydroxy-1-phenylbutan-2-yl)-N3-methyl-N3-((4-methylthiazol-2-yl)methyl)isophthalamide; N1-((2S,3R)-4-(1-(3-(difluoromethyl)phenyl)ethylamino)-3-hydroxy-1-phenylbutan-2-yl)-N3-methyl-N3-((4-methylthiazol-2-yl)methyl)isophthalamide; N1-((2S,3R)-3-hydroxy-4-(3-(1-hydroxyethyl)benzylamino)-1-phenylbutan-2-yl)-N3-methyl-N3-((4-methylthiazol-2-yl)methyl)isophthalamide; N1-((2S,3R)-3-hydroxy-1-phenyl-4-(3-(prop-1-en-2-yl)benzylamino)butan-2-yl)-N3-methyl-N3-((4-methylthiazol-2-yl)methyl)isophthalamide; N1-((2S,3R)-4-(3-(1-chloroethyl)benzylamino)-3-hydroxy-1-phenylbutan-2-yl)-N3-methyl-N3-((4-methylthiazol-2-yl)methyl)isophthalamide; 5-(fluoromethyl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-4-(3-(2-hydroxypropan-2-yl)benzylamino)-1-phenylbutan-2-yl)-N3-methyl-N3-((4-methylthiazol-2-yl)methyl)isophthalamide; N1-((2S,3R)-4-((5-tert-butylpyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-((5-phenylisoxazol-3-yl)methylamino)butan-2-yl)-N3-methyl-N3-((4-methylthiazol-2-yl)methyl)isophthalamide; 5-(difluoromethyl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-1-(3,5-difluorophenyl)-3-hydroxy-4-((5-isopropylpyridin-3-yl)methylamino)butan-2-yl)-N3-methyl-N3-((4-methylthiazol-2-yl)methyl)isophthalamide; N1-((2S,3R)-4-((5-(1,1-difluoroethyl)pyridin-3-yl)methylamino)-3-hydroxy-1-phenylbutan-2-yl)-N3-methyl-N3-((4-methylthiazol-2-yl)methyl)isophthalamide; 5-(dimethylamino)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((5-tert-butylpyridin-3-yl)methylamino)-3-hydroxy-1-phenylbutan-2-yl)-5-(dimethylamino)-N3-methyl-N3-((4-methylthiazol-2-yl)methyl)isophthalamide; N1-((2S,3R)-4-(3-(1,1-difluoroethyl)benzylamino)-3-hydroxy-1-phenylbutan-2-yl)-5-(dimethylamino)-N3-methyl-N3-((4-methylthiazol-2-yl)methyl)isophthalamide; N1-((2S,3R)-3-hydroxy-4-(3-methoxybenzylamino)-1-phenylbutan-2-yl)-N3-methyl-N3-((4-methylpyrimidin-2-yl)methyl)isophthalamide; N1-((2S,3R)-4-(3-cyanobenzylamino)-3-hydroxy-1-phenylbutan-2-yl)-N3-methyl-N3-((4-methylthiazol-2-yl)methyl)isophthalamide; 2′-cyano-N3-((2S,3R)-3-hydroxy-4-(3-methoxybenzylamino)-1-phenylbutan-2-yl)-N5-methyl-N5-((4-methylthiazol-2-yl)methyl)biphenyl-3,5-dicarboxamide; 2′-cyano-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide; N1-((2S,3R)-4-((5-bromopyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-(2-(3-methoxyphenyl)propan-2-ylamino)-1-phenylbutan-2-yl)-N3-methyl-N3-((4-methylthiazol-2-yl)methyl)isophthalamide; N1-((2S,3R)-4-((5-cyanopyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-(pyridin-2-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-2-yl)isophthalamide; N1-((2S,3R)-3-hydroxy-4-((5-isopropylpyridin-3-yl)methylamino)-1-(thiazol-4-yl)butan-2-yl)-N3-methyl-N3-((4-methylthiazol-2-yl)methyl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-(pyridin-3-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-3-yl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-(pyridin-4-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-4-yl)isophthalamide; N3-((2S,3R)-3-hydroxy-4-(3-methoxybenzylamino)-1-phenylbutan-2-yl)-2′-methoxy-N5-methyl-N5-((4-methylthiazol-2-yl)methyl)biphenyl-3,5-dicarboxamide; N1-((2S,3R)-3-hydroxy-4-((5-isopropylpyridin-3-yl)methylamino)-1-phenylbutan-2-yl)-5-(oxazol-2-yl)-N3-((R)-1-phenylethyl)isophthalamide; N1-((2S,3R)-3-hydroxy-4-(3-methoxybenzylamino)-1-phenylbutan-2-yl)-5-(oxazol-2-yl)-N3-((R)-1-phenylethyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyrazin-2-yl)isophthalamide; N1-((2S,3R)-4-((5-chloropyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(pyrazin-2-yl)isophthalamide; N1-((2S,3R)-4-((5-fluoropyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-(3-(trifluoromethyl)benzylamino)butan-2-yl)-N3,5-dimethyl-N3-((4-methylthiazol-2-yl)methyl)isophthalamide; N1-((2S,3R)-4-(3,5-dichlorobenzylamino)-3-hydroxy-1-phenylbutan-2-yl)-N3-methyl-N3-((4-methylthiazol-2-yl)methyl)isophthalamide; 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoic acid; N1-((2S,3R)-3-hydroxy-4-((5-isopropylpyridin-3-yl)methylamino)-1-phenylbutan-2-yl)-5-(1-hydroxyethyl)-N3-methyl-N3-((4-methylthiazol-2-yl)methyl)isophthalamide; methyl 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoate; N1-((2S,3R)-3-hydroxy-4-((5-isopropylpyridin-3-yl)methylamino)-1-phenylbutan-2-yl)-N3-methyl-N3-((3-methyl-1,2,4-oxadiazol-5-yl)methyl)isophthalamide; N1-((2S,3R)-3-hydroxy-4-((5-isopropylpyridin-3-yl)methylamino)-1-phenylbutan-2-yl)-N3-methyl-N3-((5-methyl-1,2,4-oxadiazol-3-yl)methyl)isophthalamide; 5-(difluoromethyl)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((5-(1,1-difluoroethyl)pyridin-3-yl)methylamino)-3-hydroxy-1-phenylbutan-2-yl)-5-(difluoromethyl)-N3-methyl-N3-((4-methylthiazol-2-yl)methyl)isophthalamide; 5-(difluoromethyl)-N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide; N1-((2S,3R)-4-((5-(1,1-difluoroethyl)pyridin-3-yl)methylamino)-3-hydroxy-1-phenylbutan-2-yl)-5-(dimethylamino)-N3-methyl-N3-((4-methylthiazol-2-yl)methyl)isophthalamide; 5-(dimethylamino)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide; 5-(dimethylamino)-N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)isophthalamide; 5-(difluoromethyl)-N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)isophthalamide; N1-((2S,3R)-3-hydroxy-1-phenyl-4-(3-(trifluoromethyl)benzylamino)butan-2-yl)-5-isopropyl-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-isopropyl-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-5-isopropyl-N3-methyl-N3-((4-methylthiazol-2-yl)methyl)isophthalamide; N1-((4-(fluoromethyl)thiazol-2-yl)methyl)-N3-((2S,3R)-3-hydroxy-4-((5-isopropylpyridin-3-yl)methylamino)-1-phenylbutan-2-yl)-N1-methylisophthalamide; N1-((2S,3R)-3-hydroxy-4-((5-((Z)-1-methoxyprop-1-en-2-yl)pyridin-3-yl)methylamino)-1-phenylbutan-2-yl)-N3-methyl-N3-((4-methylthiazol-2-yl)methyl)isophthalamide; N1-((4-(fluoromethyl)thiazol-2-yl)methyl)-N3-((2S,3R)-3-hydroxy-1-phenyl-4-((5-(trifluoromethyl)pyridin-3-yl)methylamino)butan-2-yl)-N1-methylisophthalamide; N1-((4-(fluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide; N1-((2S,3R)-4-((5-(1,1-dimethoxypropan-2-yl)pyridin-3-yl)methylamino)-3-hydroxy-1-phenylbutan-2-yl)-N3-methyl-N3-((4-methylthiazol-2-yl)methyl)isophthalamide; N1-((4-(difluoromethyl)thiazol-2-yl)methyl)-N3-((2S,3R)-3-hydroxy-4-((5-isopropylpyridin-3-yl)methylamino)-1-phenylbutan-2-yl)-N1-methylisophthalamide; N1-((4-(difluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-2-yl)isophthalamide; N1-((2S,3R)-3-hydroxy-4-((R)-1-(3-methoxyphenyl)ethylamino)-1-phenylbutan-2-yl)-N3-methyl-N3-((4-methylthiazol-2-yl)methyl)-5-(oxazol-2-yl)isophthalamide; N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)-5-(oxazol-2-yl)isophthalamide; N1-((2S,3R)-3-hydroxy-4-(3-methoxybenzylamino)-1-phenylbutan-2-yl)-5-(3-hydroxypyrrolidin-1-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-(3-hydroxypyrrolidin-1-yl)-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)-5-(1H-pyrrol-1-yl)isophthalamide; N1-((2S,3R)-3-hydroxy-4-(3-methoxybenzylamino)-1-phenylbutan-2-yl)-5-(1H-imidazol-1-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-(1H-imidazol-1-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-(1H-imidazol-2-yl)-N3-methyl-N3-((4-methylthiazol-2-yl)methyl)isophthalamide; 2-hydroxy-N1-((2S,3R)-3-hydroxy-4-(3-methoxybenzylamino)-1-phenylbutan-2-yl)-N3-methyl-N3-((4-methylthiazol-2-yl)methyl)isophthalamide; 2-hydroxy-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 5-(3,3-dimethoxypyrrolidin-1-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-4-((4-methylthiazol-2-yl)methylamino)-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-5-(1-methyl-1H-imidazol-2-yl)-N3-((4-methylthiazol-2-yl)methyl)isophthalamide; N1-((2S,3R)-4-(3-cyano-5-isopropylbenzylamino)-3-hydroxy-1-phenylbutan-2-yl)-N3-methyl-N3-((4-methylthiazol-2-yl)methyl)isophthalamide; N1-((2S,3R)-4-(5-chloro-2-fluorobenzylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-((4-(trifluoromethyl)pyridin-2-yl)methylamino)butan-2-yl)-N3-methyl-N3-((4-methylthiazol-2-yl)methyl)isophthalamide; N1-((2S,3R)-3-hydroxy-4-((5-(isopropylamino)pyridin-3-yl)methylamino)-1-phenylbutan-2-yl)-N3-methyl-N3-((4-methylthiazol-2-yl)methyl)isophthalamide; 4′-(dimethylamino)-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide; N1-((2S,3R)-4-(3-bromo-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-4-(3-cyano-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-4-(biphenyl-3-ylmethylamino)-3-hydroxy-1-phenylbutan-2-yl)-N3-methyl-N3-((4-methylthiazol-2-yl)methyl)isophthalamide; N1-((2S,3R)-4-(3-tert-butylbenzylamino)-3-hydroxy-1-phenylbutan-2-yl)-5-(dimethylamino)-N3-methyl-N3-((4-methylthiazol-2-yl)methyl)isophthalamide; 3′-chloro-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide; N1-((2S,3R)-3-hydroxy-1-phenyl-4-(3-(pyridin-4-yl)-5-(trifluoromethyl)benzylamino)butan-2-yl)-N3-methyl-N3-((4-methylthiazol-2-yl)methyl)isophthalamide; N1-((2S,3R)-4-(3-acetamido-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-3-hydroxy-4-(2-methoxy-5-(trifluoromethyl)benzylamino)-1-phenylbutan-2-yl)-N3-methyl-N3-((4-methylthiazol-2-yl)methyl)isophthalamide; N1-((2S,3R)-3-hydroxy-4-(3-(methylamino)-5-(trifluoromethyl)benzylamino)-1-phenylbutan-2-yl)-N3-methyl-N3-((4-methylthiazol-2-yl)methyl)isophthalamide; N1-((2S,3R)-3-hydroxy-4-((1-methyl-1H-pyrazol-4-yl)methylamino)-1-phenylbutan-2-yl)-N3-methyl-N3-((4-methylthiazol-2-yl)methyl)isophthalamide; N1-((2S,3R)-3-hydroxy-1-phenyl-4-(5-(prop-1-en-2-yl)nicotinamido)butan-2-yl)-N3-methyl-N3-((4-methylthiazol-2-yl)methyl)isophthalamide; N1-((2S,3R)-3-hydroxy-4-(2-hydroxy-3-(trifluoromethyl)benzylamino)-1-phenylbutan-2-yl)-N3-methyl-N3-((4-methylthiazol-2-yl)methyl)isophthalamide; 3-(((2R,3S)-2-hydroxy-3-(3-(methyl((4-methylthiazol-2-yl)methyl)carbamoyl)benzamido)-4-phenylbutylamino)methyl)-5-isopropylphenyl acetate; N1-((2S,3R)-3-hydroxy-4-((5-isopropylpyridin-3-yl)methylamino)-1-phenylbutan-2-yl)-4-methyl-N3-((4-methylthiazol-2-yl)methyl)isophthalamide; N3-((2S,3R)-3-hydroxy-4-((5-isopropylpyridin-3-yl)methylamino)-1-phenylbutan-2-yl)-N1,4-dimethyl-N1-((4-methylthiazol-2-yl)methyl)isophthalamide; N1-((2S,3R)-3-hydroxy-4-(3-isopropyl-5-(N-methylsulfamoyl)benzylamino)-1-phenylbutan-2-yl)-N3-methyl-N3-((4-methylthiazol-2-yl)methyl)isophthalamide; 3-(((2R,3S)-2-hydroxy-3-(3-(methyl((4-methylthiazol-2-yl)methyl)carbamoyl)benzamido)-4-phenylbutylamino)methyl)-5-isopropylphenyl dimethyl phosphate; 5-acetyl-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide; 5-(furan-2-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-5-(1-methyl-1H-pyrrol-2-yl)-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-5-(1-methyl-1H-pyrazol-4-yl)-N3-((4-methylthiazol-2-yl)methyl)isophthalamide; 5-(6-fluoropyridin-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 5-(furan-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 5-(furan-2-yl)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide; N1-((2S,3R)-4-((5-acetylpyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrazol-4-yl)isophthalamide; 3-(((2R,3S)-2-hydroxy-3-(3-(methyl((4-methylthiazol-2-yl)methyl)carbamoyl)benzamido)-4-phenylbutylamino)methyl)-5-(prop-1-en-2-yl)pyridine 1-oxide; N1-((2S,3R)-4-((5-(2-fluoropropan-2-yl)pyridin-3-yl)methylamino)-3-hydroxy-1-phenylbutan-2-yl)-5-(furan-3-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((5-methyl-1,3,4-oxadiazol-2-yl)methyl)isophthalamide; N1-((2S,3R)-3-hydroxy-4-(3-methoxybenzylamino)-1-phenylbutan-2-yl)-5-(3-hydroxycyclopentyl)-N3-methyl-N3-((4-methylthiazol-2-yl)methyl)isophthalamide; 5-acetyl-N1-((2S,3R)-1-(3,5-difluorophenyl)-3-hydroxy-4-(3-methoxybenzylamino)butan-2-yl)-N3-((R)-1-phenylethyl)isophthalamide; 5-acetyl-N1-((2S,3R)-3-hydroxy-4-((5-isopropylpyridin-3-yl)methylamino)-1-phenylbutan-2-yl)-N3-((R)-1-phenylethyl)isophthalamide; N1-((2S,3R)-3-hydroxy-4-(3-methoxyphenylsulfonamido)-1-phenylbutan-2-yl)-N3-methyl-N3-((4-methylthiazol-2-yl)methyl)isophthalamide; N1-((2S,3R)-3-hydroxy-1-phenyl-4-(3-(trifluoromethyl)phenylsulfonamido)butan-2-yl)-N3-methyl-N3-((4-methylthiazol-2-yl)methyl)isophthalamide; 3-(((2R,3S)-2-hydroxy-3-(3-(methyl((4-methylthiazol-2-yl)methyl)carbamoyl)benzamido)-4-phenylbutylamino)methyl)-5-isopropylphenyl 2-(dimethylamino)acetate; N1-((2S,3R)-3-hydroxy-4-(((5-isopropylpyridin-3-yl)methyl)(methyl)amino)-1-phenylbutan-2-yl)-N3-methyl-N3-((4-methylthiazol-2-yl)methyl)isophthalamide; N1-((2S,3R)-3-hydroxy-4-((5-(1-hydroxypropan-2-yl)pyridin-3-yl)methylamino)-1-phenylbutan-2-yl)-N3-methyl-N3-((4-methylthiazol-2-yl)methyl)isophthalamide; N1-((4-bromothiazol-2-yl)methyl)-N3-((2S,3R)-3-hydroxy-4-((5-isopropylpyridin-3-yl)methylamino)-1-phenylbutan-2-yl)-N1-methylisophthalamide; N1-((2S,3R)-3-hydroxy-4-((5-isopropylpyridin-3-yl)methylamino)-1-phenylbutan-2-yl)-N3-((4-(hydroxymethyl)thiazol-2-yl)methyl)-N3-methylisophthalamide; tert-butyl 3′-((2S,3R)-3-hydroxy-1-phenyl-4-(3-(trifluoromethyl)benzylamino)butan-2-ylcarbamoyl)-5′-(methyl((4-methylthiazol-2-yl)methyl)carbamoyl)biphenyl-3-yl(methyl)carbamate; N1-((2S,3R)-3-hydroxy-4-((5-isopropylpyridin-3-yl)methylamino)-1-phenylbutan-2-yl)-N3-methyl-N3-(thiazol-2-ylmethyl)isophthalamide; N1-((2S,3R)-3-hydroxy-1-phenyl-4-((5-(prop-1-en-2-yl)pyridin-3-yl)methylamino)butan-2-yl)-N3-((4-(hydroxymethyl)thiazol-2-yl)methyl)-N3-methylisophthalamide; N1-((2S,3R)-4-((1-ethyl-1H-pyrazol-4-yl)methylamino)-3-hydroxy-1-phenylbutan-2-yl)-N3-methyl-N3-((4-methylthiazol-2-yl)methyl)isophthalamide; or a pharmaceutically acceptable salt or solvate thereof.

or a stereoisomer, mixture of stereoisomers, crystalline form, non-crystalline form, hydrate, solvate, or salt thereof.

In other embodiments, the β-secretase inhibitor compound is: N1-((2S,3R)-3-hydroxy-4-(3-methoxybenzylamino)-1-phenylbutan-2-yl)-N3-methyl-N3-((4-methylthiazol-2-yl)methyl)-5-(oxazol-2-yl)isophthalamide; methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)oxazole-4-carboxylate; N1-((2S,3R)-3-hydroxy-4-(3-methoxybenzylamino)-1-phenylbutan-2-yl)-N3-methyl-N3-((4-methylthiazol-2-yl)methyl)-5-(1H-pyrrol-1-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrrol-1-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-2-yl)isophthalamide; N1-((2S,3R)-4-((2-amino-6-isopropylpyrimidin-4-yl)methylamino)-3-hydroxy-1-phenylbutan-2-yl)-N3-methyl-N3-((4-methylthiazol-2-yl)methyl)-5-(oxazol-2-yl)isophthalamide; N1-cyclopropyl-N3-((2S,3R)-3-hydroxy-4-((5-isopropylpyridin-3-yl)methylamino)-1-phenylbutan-2-yl)-N1-((4-methylthiazol-2-yl)methyl)-5-(oxazol-2-yl)isophthalamide; N1-((2S,3R)-3-hydroxy-4-((5-isopropylpyridin-3-yl)methylamino)-1-phenylbutan-2-yl)-N3-((4-methylthiazol-2-yl)methyl)-5-(oxazol-2-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-5-yl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-5-yl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-(pyridin-2-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-2-yl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-(pyridin-3-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-3-yl)isophthalamide; N1-((2S,3R)-3-hydroxy-4-(3-methoxybenzylamino)-1-phenylbutan-2-yl)-N3-methyl-N3-((4-methylthiazol-2-yl)methyl)-5-(pyridin-4-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-4-yl)isophthalamide; N1-((2S,3R)-3-hydroxy-4-((5-isopropylpyridin-3-yl)methylamino)-1-phenylbutan-2-yl)-5-(oxazol-2-yl)-N3-((R)-1-phenylethyl)isophthalamide; N1-((2S,3R)-3-hydroxy-4-(3-methoxybenzylamino)-1-phenylbutan-2-yl)-5-(oxazol-2-yl)-N3-((R)-1-phenylethyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyrazin-2-yl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(pyrazin-2-yl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-2-yl)isophthalamide; N1-((2S,3R)-3-hydroxy-4-((R)-1-(3-methoxyphenyl)ethylamino)-1-phenylbutan-2-yl)-N3-methyl-N3-((4-methylthiazol-2-yl)methyl)-5-(oxazol-2-yl)isophthalamide; N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)-5-(oxazol-2-yl)isophthalamide; N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)-5-(1H-pyrrol-1-yl)isophthalamide; N1-((2S,3R)-3-hydroxy-4-(3-methoxybenzylamino)-1-phenylbutan-2-yl)-5-(1H-imidazol-1-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-(1H-imidazol-1-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-(1H-imidazol-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-5-(1-methyl-1H-imidazol-2-yl)-N3-((4-methylthiazol-2-yl)methyl)isophthalamide; 5-(furan-2-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-5-(1-methyl-1H-pyrrol-2-yl)-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-5-(1-methyl-1H-pyrazol-4-yl)-N3-((4-methylthiazol-2-yl)methyl)isophthalamide; 5-(6-fluoropyridin-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 5-(furan-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 5-(furan-2-yl)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrazol-4-yl)isophthalamide; N1-((2S,3R)-4-((5-(2-fluoropropan-2-yl)pyridin-3-yl)methylamino)-3-hydroxy-1-phenylbutan-2-yl)-5-(furan-3-yl)-N3-methyl-N3-((4-methylthiazol-2-yl)methyl)isophthalamide; or a stereoisomer, mixture of stereoisomers, crystalline form, non-crystalline form, hydrate, solvate, or salt thereof.

In other embodiments, the β-secretase inhibitor compound is: N1-((2S,3R)-3-hydroxy-1-phenyl-4-(pyridin-3-ylmethylamino)butan-2-yl)-N3-methyl-N3-((4-methylthiazol-2-yl)methyl)isophthalamide; N1-((2S,3R)-3-hydroxy-1-phenyl-4-(pyridin-4-ylmethylamino)butan-2-yl)-N3-methyl-N3-((4-methylthiazol-2-yl)methyl)isophthalamide; N1-((2S,3R)-3-hydroxy-1-phenyl-4-(pyridin-2-ylmethylamino)butan-2-yl)-N3-methyl-N3-((4-methylthiazol-2-yl)methyl)isophthalamide; N1-((2S,3R)-3-hydroxy-1-phenyl-4-(1-(pyridin-3-yl)ethylamino)butan-2-yl)-N3-methyl-N3-((4-methylthiazol-2-yl)methyl)isophthalamide; N1-((2S,3R)-3-hydroxy-4-((6-methylpyridin-2-yl)methylamino)-1-phenylbutan-2-yl)-N3-methyl-N3-((4-methylthiazol-2-yl)methyl)isophthalamide; N1-((2R,3S)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-1-phenyl-4-((6-(trifluoromethyl)pyridin-3-yl)methylamino)butan-2-yl)-N3-methyl-N3-((4-methylthiazol-2-yl)methyl)isophthalamide; N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((4-isopropylpyridin-2-yl)methylamino)-1-phenylbutan-2-yl)-N3-methyl-N3-((4-methylthiazol-2-yl)methyl)isophthalamide; N1-((2S,3R)-3-hydroxy-4-((6-isopropylpyridin-2-yl)methylamino)-1-phenylbutan-2-yl)-N3-methyl-N3-((4-methylthiazol-2-yl)methyl)isophthalamide; N1-((2S,3R)-3-hydroxy-4-((2-isopropylpyridin-4-yl)methylamino)-1-phenylbutan-2-yl)-N3-methyl-N3-((4-methylthiazol-2-yl)methyl)isophthalamide; methyl 6-(((2R,3S)-2-hydroxy-3-(3-(methyl((4-methylthiazol-2-yl)methyl)carbamoyl)benzamido)-4-phenylbutylamino)methyl)-2-methylnicotinate; N1-((2S,3R)-3-hydroxy-4-((5-(hydroxymethyl)-6-methylpyridin-2-yl)methylamino)-1-phenylbutan-2-yl)-N3-methyl-N3-((4-methylthiazol-2-yl)methyl)isophthalamide; N1-((2S,3R)-4-((5-(dimethylamino)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((2-fluoro-5-isopropylpyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((5-isopentylpyridin-3-yl)methylamino)-1-phenylbutan-2-yl)-N3-methyl-N3-((4-methylthiazol-2-yl)methyl)isophthalamide; N1-((2S,3R)-3-hydroxy-4-((6-methyl-5-(methylthiomethyl)pyridin-2-yl)methylamino)-1-phenylbutan-2-yl)-N3-methyl-N3-((4-methylthiazol-2-yl)methyl)isophthalamide; N1-((2S,3R)-4-((2,6-diisopropylpyridin-4-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((R)-1-(4-methylthiazol-2-yl)ethyl)isophthalamide; N1-((2S,3R)-4-((5-cyclopropylpyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-((5-(prop-1-en-2-yl)pyridin-3-yl)methylamino)butan-2-yl)-N3-methyl-N3-((4-methylthiazol-2-yl)methyl)isophthalamide; N1-cyclopropyl-N3-((2S,3R)-3-hydroxy-1-phenyl-4-((5-(trifluoromethyl)pyridin-3-yl)methylamino)butan-2-yl)-N1-((4-methylthiazol-2-yl)methyl)isophthalamide; N1-((2S,3R)-3-hydroxy-4-((5-isopropyl-2,6-dimethylpyridin-3-yl)methylamino)-1-phenylbutan-2-yl)-N3-((4-methylthiazol-2-yl)methyl)isophthalamide; N1-((2S,3R)-3-hydroxy-4-((6-methyl-5-(methylsulfonylmethyl)pyridin-2-yl)methylamino)-1-phenylbutan-2-yl)-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methyloxazol-2-yl)methyl)isophthalamide; N1-((2S,3R)-3-hydroxy-4-((5-isopropylpyridin-3-yl)methylamino)-1-phenylbutan-2-yl)-N3,5-dimethyl-N3-((4-methylthiazol-2-yl)methyl)isophthalamide; N1-((2S,3R)-3-hydroxy-4-((2-isopropylpyridin-4-yl)methylamino)-1-phenylbutan-2-yl)-N3,5-dimethyl-N3-((4-methylthiazol-2-yl)methyl)isophthalamide; N1-cyclopropyl-N3-((2S,3R)-3-hydroxy-4-((5-isopropylpyridin-3-yl)methylamino)-1-phenylbutan-2-yl)-5-methyl-N1-((4-methylthiazol-2-yl)methyl)isophthalamide; N1-((2S,3R)-3-hydroxy-4-((2-isopropylpyridin-4-yl)methylamino)-1-phenylbutan-2-yl)-5-methoxy-N3-methyl-N3-((4-methylthiazol-2-yl)methyl)isophthalamide; N1-((2S,3R)-3-hydroxy-4-((2-isopropylpyridin-4-yl)methylamino)-1-phenylbutan-2-yl)-5-methoxy-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-methoxy-N3-methyl-N3-((4-methylthiazol-2-yl)methyl)isophthalamide; 3-((2S,3R)-3-hydroxy-4-((5-isopropylpyridin-3-yl)methylamino)-1-phenylbutan-2-ylcarbamoyl)-5-(methyl((4-methylthiazol-2-yl)methyl)carbamoyl)phenyl methanesulfonate; N1-((2S,3R)-4-((5-(2-fluoropropan-2-yl)pyridin-3-yl)methylamino)-3-hydroxy-1-phenylbutan-2-yl)-5-(N-methylmethylsulfonamido)-N3-((R)-1-phenylethyl)isophthalamide; N1-((2S,3R)-3-hydroxy-1-phenyl-4-((5-(trifluoromethyl)pyridin-3-yl)methylamino)butan-2-yl)-5-(N-methylmethylsulfonamido)-N3-((R)-1-phenylethyl)isophthalamide; 5-(dimethylamino)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 5-(diethylamino)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyrrolidin-1-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(2-oxopyrrolidin-1-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(piperidin-1-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrrol-1-yl)isophthalamide; N1-((2S,3R)-3-hydroxy-4-(1-(5-isopropylpyridin-3-yl)ethylamino)-1-phenylbutan-2-yl)-N3-methyl-N3-((4-methylthiazol-2-yl)methyl)isophthalamide; N1-((2S,3R)-3-hydroxy-4-((S)-1-(5-isopropylpyridin-3-yl)ethylamino)-1-phenylbutan-2-yl)-N3-methyl-N3-((4-methylthiazol-2-yl)methyl)isophthalamide; N1-((2S,3R)-3-hydroxy-4-((R)-1-(5-isopropylpyridin-3-yl)ethylamino)-1-phenylbutan-2-yl)-N3-methyl-N3-((4-methylthiazol-2-yl)methyl)isophthalamide; N1-((2S,3R)-3-hydroxy-4-((5-isopropyl-2-methylpyridin-3-yl)methylamino)-1-phenylbutan-2-yl)-N3-methyl-N3-((4-methylthiazol-2-yl)methyl)isophthalamide; N1-((2S,3S)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2R,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-2-yl)isophthalamide; N1-cyclopropyl-N3-((2S,3R)-3-hydroxy-4-((5-isopropylpyridin-3-yl)methylamino)-1-phenylbutan-2-yl)-N1-(4-methylthiazol-2-yl)methyl)-5-(oxazol-2-yl)isophthalamide; N1-((2S,3R)-3-hydroxy-4-((5-isopropylpyridin-3-yl)methylamino)-1-phenylbutan-2-yl)-5-(hydroxymethyl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-((4-methylthiazol-2-yl)methyl)-5-(oxazol-2-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-5-yl)isophthalamide; 5-acetyl-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 5-(fluoromethyl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-4-((5-tert-butylpyridin-3-yl)methylamino)-3-hydroxy-1-phenylbutan-2-yl)-N3-methyl-N3-((4-methylthiazol-2-yl)methyl)isophthalamide; 5-(difluoromethyl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-1-(3,5-difluorophenyl)-3-hydroxy-4-((5-isopropylpyridin-3-yl)methylamino)butan-2-yl)-N3-methyl-N3-((4-methylthiazol-2-yl)methyl)isophthalamide; N1-((2S,3R)-4-((5-(1,1-difluoroethyl)pyridin-3-yl)methylamino)-3-hydroxy-1-phenylbutan-2-yl)-N3-methyl-N3-((4-methylthiazol-2-yl)methyl)isophthalamide; 5-(dimethylamino)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((5-tert-butylpyridin-3-yl)methylamino)-3-hydroxy-1-phenylbutan-2-yl)-5-(dimethylamino)-N3-methyl-N3-((4-methylthiazol-2-yl)methyl)isophthalamide; 2′-cyano-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide; N1-((2S,3R)-4-((5-bromopyridin-3-yl)methylamino)-3-hydroxy-1-phenylbutan-2-yl)-N3-methyl-N3-((4-methylthiazol-2-yl)methyl)isophthalamide; N1-((2S,3R)-4-((5-cyanopyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-2-yl)isophthalamide; N1-((2S,3R)-3-hydroxy-4-((5-isopropylpyridin-3-yl)methylamino)-1-(thiazol-4-yl)butan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-3-yl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-4-yl)isophthalamide; N1-((2S,3R)-3-hydroxy-4-((5-isopropylpyridin-3-yl)methylamino)-1-phenylbutan-2-yl)-5-(oxazol-2-yl)-N3-((R)-1-phenylethyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyrazin-2-yl)isophthalamide; N1-((2S,3R)-4-((5-chloropyridin-3-yl)methylamino)-3-hydroxy-1-phenylbutan-2-yl)-N3-methyl-N3-((4-methylthiazol-2-yl)methyl)isophthalamide; N1-((2S,3R)-4-((5-fluoropyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-(1-hydroxyethyl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((3-methyl-1,2,4-oxadiazol-5-yl)methyl)isophthalamide; N1-((2S,3R)-3-hydroxy-4-((5-isopropylpyridin-3-yl)methylamino)-1-phenylbutan-2-yl)-N3-methyl-N3-((5-methyl-1,2,4-oxadiazol-3-yl)methyl)isophthalamide; 5-(difluoromethyl)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((5-(1,1-difluoroethyl)pyridin-3-yl)methylamino)-3-hydroxy-1-phenylbutan-2-yl)-5-(difluoromethyl)-N3-methyl-N3-((4-methylthiazol-2-yl)methyl)isophthalamide; 5-(difluoromethyl)-N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide; N1-((2S,3R)-4-((5-(1,1-difluoroethyl)pyridin-3-yl)methylamino)-3-hydroxy-1-phenylbutan-2-yl)-5-(dimethylamino)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-isopropyl-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-5-isopropyl-N3-methyl-N3-((4-methylthiazol-2-yl)methyl)isophthalamide; N1-((4-(fluoromethyl)thiazol-2-yl)methyl)-N3-((2S,3R)-3-hydroxy-4-((5-isopropylpyridin-3-yl)methylamino)-1-phenylbutan-2-yl)-N1-methylisophthalamide; N1-((2S,3R)-3-hydroxy-4-((5-((Z)-1-methoxyprop-1-en-2-yl)pyridin-3-yl)methylamino)-1-phenylbutan-2-yl)-N3-methyl-N3-((4-methylthiazol-2-yl)methyl)isophthalamide; N1-((4-(fluoromethyl)thiazol-2-yl)methyl)-N3-((2S,3R)-3-hydroxy-1-phenyl-4-((5-(trifluoromethyl)pyridin-3-yl)methylamino)butan-2-yl)-N1-methylisophthalamide; N1-((4-(fluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide; N1-((2S,3R)-4-((5-(1,1-dimethoxypropan-2-yl)pyridin-3-yl)methylamino)-3-hydroxy-1-phenylbutan-2-yl)-N3-methyl-N3-((4-methylthiazol-2-yl)methyl)isophthalamide; N1-((4-(difluoromethyl)thiazol-2-yl)methyl)-N3-((2S,3R)-3-hydroxy-4-((5-isopropylpyridin-3-yl)methylamino)-1-phenylbutan-2-yl)-N1-methylisophthalamide; N1-((4-(difluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide; N1-((2S,3R)-3-hydroxy-4-((5-isopropylpyridin-3-yl)methylamino)-1-phenylbutan-2-yl)-5-(3-hydroxypyrrolidin-1-yl)-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)-5-(1H-pyrrol-1-yl)isophthalamide; N1-((2S,3R)-3-hydroxy-4-((5-isopropylpyridin-3-yl)methylamino)-1-phenylbutan-2-yl)-5-(1H-imidazol-1-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-5-(1H-imidazol-2-yl)-N3-methyl-N3-((4-methylthiazol-2-yl)methyl)isophthalamide; 2-hydroxy-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 5-(3,3-dimethoxypyrrolidin-1-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-5-(1-methyl-1H-imidazol-2-yl)-N3-((4-methylthiazol-2-yl)methyl)isophthalamide; N1-((2S,3R)-3-hydroxy-1-phenyl-4-((4-(trifluoromethyl)pyridin-2-yl)methylamino)butan-2-yl)-N3-methyl-N3-((4-methylthiazol-2-yl)methyl)isophthalamide; N1-((2S,3R)-3-hydroxy-4-((5-(isopropylamino)pyridin-3-yl)methylamino)-1-phenylbutan-2-yl)-N3-methyl-N3-((4-methylthiazol-2-yl)methyl)isophthalamide; 4′-(dimethylamino)-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide; 3′-chloro-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide; N1-((2S,3R)-3-hydroxy-1-phenyl-4-(5-(prop-1-en-2-yl)nicotinamido)butan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-4-methyl-N3-((4-methylthiazol-2-yl)methyl)isophthalamide; N3-((2S,3R)-3-hydroxy-4-((5-isopropylpyridin-3-yl)methylamino)-1-phenylbutan-2-yl)-N1,4-dimethyl-N1-((4-methylthiazol-2-yl)methyl)isophthalamide; 5-(furan-2-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-5-(1-methyl-1H-pyrrol-2-yl)-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-5-(1-methyl-1H-pyrazol-4-yl)-N3-((4-methylthiazol-2-yl)methyl)isophthalamide; 5-(6-fluoropyridin-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; 5-(furan-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide; N1-((2S,3R)-4-((5-acetylpyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrazol-4-yl)isophthalamide; N1-((2S,3R)-4-((5-(2-fluoropropan-2-yl)pyridin-3-yl)methylamino)-3-hydroxy-1-phenylbutan-2-yl)-5-(furan-3-yl)-N3-methyl-N3-((4-methylthiazol-2-yl)methyl)isophthalamide; N1-((2S,3R)-3-hydroxy-4-((5-isopropylpyridin-3-yl)methylamino)-1-phenylbutan-2-yl)-N3-methyl-N3-((5-methyl-1,3,4-oxadiazol-2-yl)methyl)isophthalamide; 5-acetyl-N1-((2S,3R)-3-hydroxy-4-((5-isopropylpyridin-3-yl)methylamino)-1-phenylbutan-2-yl)-N3-((R)-1-phenylethyl)isophthalamide; N1-((2S,3R)-3-hydroxy-4-(((5-isopropylpyridin-3-yl)methyl)(methyl)amino)-1-phenylbutan-2-yl)-N3-methyl-N3-((4-methylthiazol-2-yl)methyl)isophthalamide; or a stereoisomer, mixture of stereoisomers, crystalline form, non-crystalline form, hydrate, solvate, or salt thereof.

In other embodiments, the β-secretase inhibitor compound is: N1-(2S,3R)-4-(3-fluoro-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-4-(2-fluoro-6-methoxybenzylamino)-3-hydroxy-1-phenylbutan-2-yl)-N3-methyl-N3-((4-methylthiazol-2-yl)methyl)isophthalamide; N1-((2S,3R)-4-(3,5-bis(trifluoromethyl)benzylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-((6-(trifluoromethyl)pyridin-3-yl)methylamino)butan-2-yl)-N3-methyl-N3-((4-methylthiazol-2-yl)methyl)isophthalamide; N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((2-fluoro-5-isopropylpyridin-3-yl)methylamino)-3-hydroxy-1-phenylbutan-2-yl)-N3-methyl-N3-((4-methylthiazol-2-yl)methyl)isophthalamide; N1-cyclopropyl-N3-((2S,3R)-3-hydroxy-1-phenyl-4-(3-(trifluoromethyl)benzylamino)butan-2-yl)-N1-((4-methylthiazol-2-yl)methyl)isophthalamide; N1-cyclopropyl-N3-((2S,3R)-3-hydroxy-1-phenyl-4-(3-(trifluoromethoxy)benzylamino)butan-2-yl)-N1-((4-methylthiazol-2-yl)methyl)isophthalamide; N1-cyclopropyl-N3-((2S,3R)-3-hydroxy-1-phenyl-4-((5-(trifluoromethyl)pyridin-3-yl)methylamino)butan-2-yl)-N1-((4-methylthiazol-2-yl)methyl)isophthalamide; N1-((2S,3R)-3-hydroxy-1-phenyl-4-(3-(trifluoromethyl)benzylamino)butan-2-yl)-5-methoxy-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-5-(N-methylmethylsulfonamido)-N3-((R)-1-phenylethyl)isophthalamide; N1-((2S,3R)-3-hydroxy-1-phenyl-4-((5-(trifluoromethyl)pyridin-3-yl)methylamino)butan-2-yl)-5-(N-methylmethylsulfonamido)-N3-((R)-1-phenylethyl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(2-oxopyrrolidin-1-yl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-5-yl)isophthalamide; N1-((2S,3R)-4-(3-(difluoromethyl)benzylamino)-3-hydroxy-1-phenylbutan-2-yl)-5-(N-methylmethylsulfonamido)-N3-((R)-1-phenylethyl)isophthalamide; N1-((2S,3R)-4-(3-(difluoromethyl)benzylamino)-3-hydroxy-1-phenylbutan-2-yl)-N3-methyl-N3-((4-methylthiazol-2-yl)methyl)isophthalamide; N1-((2S,3R)-4-(3-(1,1-difluoroethyl)benzylamino)-3-hydroxy-1-phenylbutan-2-yl)-5-(N-methylmethylsulfonamido)-N3-((R)-1-phenylethyl)isophthalamide; N1-((2S,3R)-4-(3-(1,1-difluoroethyl)benzylamino)-3-hydroxy-1-phenylbutan-2-yl)-N3-methyl-N3-((4-methylthiazol-2-yl)methyl)isophthalamide; N1-((2S,3R)-4-(1-(3-(difluoromethyl)phenyl)ethylamino)-3-hydroxy-1-phenylbutan-2-yl)-N3-methyl-N3-((4-methylthiazol-2-yl)methyl)isophthalamide; N1-((2S,3R)-4-((5-(1,1-difluoroethyl)pyridin-3-yl)methylamino)-3-hydroxy-1-phenylbutan-2-yl)-N3-methyl-N3-((4-methylthiazol-2-yl)methyl)isophthalamide; 5-(dimethylamino)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-4-(3-(1,1-difluoroethyl)benzylamino)-3-hydroxy-1-phenylbutan-2-yl)-5-(dimethylamino)-N3-methyl-N3-((4-methylthiazol-2-yl)methyl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(pyrazin-2-yl)isophthalamide; N1-((2S,3R)-4-((5-fluoropyridin-3-yl)methylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-(3-(trifluoromethyl)benzylamino)butan-2-yl)-N3,5-dimethyl-N3-((4-methylthiazol-2-yl)methyl)isophthalamide; 5-(difluoromethyl)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((5-(1,1-difluoroethyl)pyridin-3-yl)methylamino)-3-hydroxy-1-phenylbutan-2-yl)-5-(difluoromethyl)-N3-methyl-N3-((4-methylthiazol-2-yl)methyl)isophthalamide; 5-(difluoromethyl)-N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide; N1-((2S,3R)-4-((5-(1,1-difluoroethyl)pyridin-3-yl)methylamino)-3-hydroxy-1-phenylbutan-2-yl)-5-(dimethylamino)-N3-methyl-N3-((4-methylthiazol-2-yl)methyl)isophthalamide; 5-(dimethylamino)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide; N1-((2S,3R)-3-hydroxy-1-phenyl-4-(3-(trifluoromethyl)benzylamino)butan-2-yl)-5-isopropyl-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-5-isopropyl-N3-methyl-N3-((4-methylthiazol-2-yl)methyl)isophthalamide; N1-((4-(fluoromethyl)thiazol-2-yl)methyl)-N3-((2S,3R)-3-hydroxy-1-phenyl-4-((5-(trifluoromethyl)pyridin-3-yl)methylamino)butan-2-yl)-N1-methylisophthalamide; N1-((4-(fluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide; N1-((4-(difluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-2-yl)isophthalamide; N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)-5-(1H-pyrrol-1-yl)isophthalamide; N1-((2S,3R)-4-(5-chloro-2-fluorobenzylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-((4-(trifluoromethyl)pyridin-2-yl)methylamino)butan-2-yl)-N3-methyl-N3-((4-methylthiazol-2-yl)methyl)isophthalamide; N1-((2S,3R)-4-(3-bromo-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-4-(3-cyano-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-(3-(pyridin-4-yl)-5-(trifluoromethyl)benzylamino)butan-2-yl)-N3-methyl-N3-((4-methylthiazol-2-yl)methyl)isophthalamide; N1-((2S,3R)-4-(3-acetamido-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-3-hydroxy-4-(2-methoxy-5-(trifluoromethyl)benzylamino)-1-phenylbutan-2-yl)-N3-methyl-N3-((4-methylthiazol-2-yl)methyl)isophthalamide; N1-((2S,3R)-3-hydroxy-4-(3-(methylamino)-5-(trifluoromethyl)benzylamino)-1-phenylbutan-2-yl)-N3-methyl-N3-((4-methylthiazol-2-yl)methyl)isophthalamide; N1-((2S,3R)-3-hydroxy-4-(2-hydroxy-3-(trifluoromethyl)benzylamino)-1-phenylbutan-2-yl)-N3-methyl-N3-((4-methylthiazol-2-yl)methyl)isophthalamide; 5-acetyl-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide; 5-(furan-2-yl)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-5-(furan-3-yl)-N3-methyl-N3-((4-methylthiazol-2-yl)methyl)isophthalamide; N1-((2S,3R)-3-hydroxy-1-phenyl-4-(3-(trifluoromethyl)phenylsulfonamido)butan-2-yl)-N3-methyl-N3-((4-methylthiazol-2-yl)methyl)isophthalamide; tert-butyl 3′-((2S,3R)-3-hydroxy-1-phenyl-4-(3-(trifluoromethyl)benzylamino)butan-2-ylcarbamoyl)-5′-(methyl((4-methylthiazol-2-yl)methyl)carbamoyl)biphenyl-3-yl(methyl)carbamate; or a stereoisomer, mixture of stereoisomers, crystalline form, non-crystalline form, hydrate, solvate, or salt thereof.

In other embodiments, the β-secretase inhibitor compound is: N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-5-(N-methylmethylsulfonamido)-N3-N3-((R)-1-phenylethyl)isophthalamide; 5-(dimethylamino)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; N1-((2S,3R)-4-((5-(2-fluoropropan-2-yl)pyridin-3-yl)methylamino)-3-hydroxy-1-phenylbutan-2-yl)-5-isopropyl-N3-methyl-N3-((4-methylthiazol-2-yl)methyl)isophthalamide; N1-((4-(fluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide; N1-((4-(difluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide; N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)-5-(1H-pyrrol-1-yl)isophthalamide; N1-((2S,3R)-4-((5-(2-fluoropropan-2-yl)pyridin-3-yl)methylamino)-3-hydroxy-1-phenylbutan-2-yl)-5-(furan-3-yl)-N3-methyl-N3-((4-methylthiazol-2-yl)methyl)isophthalamide; or a stereoisomer, mixture of stereoisomers, crystalline form, non-crystalline form, hydrate, solvate, or salt thereof.

In other embodiments, the β-secretase inhibitor compound is: N1-((2S,3R)-4-(3-fluoro-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-4-(3,5-bis(trifluoromethyl)benzylamino)-3-hydroxy-1-phenylbutan-2-yl)-N3-methyl-N3-((4-methylthiazol-2-yl)methyl)isophthalamide; N1-cyclopropyl-N3-((2S,3R)-3-hydroxy-1-phenyl-4-(3-(trifluoromethyl)benzylamino)butan-2-yl)-N1-((4-methylthiazol-2-yl)methyl)isophthalamide; N1-((2S,3R)-3-hydroxy-1-phenyl-4-(3-(trifluoromethyl)benzylamino)butan-2-yl)-5-methoxy-N3-methyl-N3-((4-methylthiazol-2-yl)methyl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(2-oxopyrrolidin-1-yl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-5-yl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(pyrazin-2-yl)isophthalamide; N1-((2S,3R)-3-hydroxy-1-phenyl-4-(3-(trifluoromethyl)benzylamino)butan-2-yl)-N3,5-dimethyl-N3-((4-methylthiazol-2-yl)methyl)isophthalamide; N1-((2S,3R)-3-hydroxy-1-phenyl-4-(3-(trifluoromethyl)benzylamino)butan-2-yl)-5-isopropyl-N3-methyl-N3-((4-methylthiazol-2-yl)methyl)isophthalamide; N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-2-yl)isophthalamide; N1-((2S,3R)-4-(3-bromo-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-4-(3-cyano-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-3-hydroxy-1-phenyl-4-(3-(pyridin-4-yl)-5-(trifluoromethyl)benzylamino)butan-2-yl)-N3-methyl-N3-((4-methylthiazol-2-yl)methyl)isophthalamide; N1-((2S,3R)-4-(3-acetamido-5-(trifluoromethyl)benzylamino)-3-hydroxy-1-phenylbutan-2-yl)-N3-methyl-N3-((4-methylthiazol-2-yl)methyl)isophthalamide; N1-((2S,3R)-3-hydroxy-4-(2-methoxy-5-(trifluoromethyl)benzylamino)-1-phenylbutan-2-yl)-N3-methyl-N3-((4-methylthiazol-2-yl)methyl)isophthalamide; N1-((2S,3R)-3-hydroxy-4-(3-(methylamino)-5-(trifluoromethyl)benzylamino)-1-phenylbutan-2-yl)-N3-methyl-N3-((4-methylthiazol-2-yl)methyl)isophthalamide; N1-((2S,3R)-3-hydroxy-4-(2-hydroxy-3-(trifluoromethyl)benzylamino)-1-phenylbutan-2-yl)-N3-methyl-N3-((4-methylthiazol-2-yl)methyl)isophthalamide; 5-acetyl-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide; 5-(furan-2-yl)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide; N1-((2S,3R)-3-hydroxy-1-phenyl-4-(3-(trifluoromethyl)phenylsulfonamido)butan-2-yl)-N3-methyl-N3-((4-methylthiazol-2-yl)methyl)isophthalamide; tert-butyl 3′-((2S,3R)-3-hydroxy-1-phenyl-4-(3-(trifluoromethyl)benzylamino)butan-2-ylcarbamoyl)-5′-(methyl((4-methylthiazol-2-yl)methyl)carbamoyl)biphenyl-3-yl(methyl)carbamate; or a stereoisomer, mixture of stereoisomers, crystalline form, non-crystalline form, hydrate, solvate, or salt thereof.

In some embodiments, the compounds of the present invention include any one, any combination, or all of the compounds of Example 3; or a stereoisomer, mixture of stereoisomers, crystalline form, non-crystalline form, hydrate, solvate, or salt thereof.

In another embodiment, the β-secretase inhibitor compound is: N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide; or a stereoisomer, mixture of stereoisomers, crystalline form, non-crystalline form, hydrate, solvate, or salt thereof.

A. Carrier Moieties

In U.S. Application No. 20040121947, and International Application No. PCT/US02/34324 (Publication No. WO 03/039454), which are herein incorporated by reference for all purposes, isostere β-secretase inhibitors with and without a carrier moiety were shown to effectively reduce Aβ production in tg2576 mice expressing the Swedish mutation of the human amyloid precursor protein (Hsiao, K., et al., Science 274, 99-102 (1996)). Thus, one of skill in the art will recognize that the compounds of the invention may be administered with or without a carrier moiety.

A “carrier moiety,” as used herein, refers to a chemical moiety covalently or non-covalently attached to a β-secretase inhibitor compound of the invention that enhances the ability of the compound to traverse the blood-brain barrier (BBB). The β-secretase inhibitors of the invention may be attached or conjugated to the carrier moiety by covalent interactions (e.g., peptide bonds) or by non-covalent interactions (e.g., ionic bonds, hydrogen bonds, van der Waals attractions). A covalently attached carrier moiety may be attached to any appropriate site on the compounds of the present invention (e.g., a hydroxyl group, amino group, thiol group, carboxylate group). One or more carrier moieties may be used on a compound of the invention. Multiple carrier moieties on a compound may be identical (e.g. multiple peptidyl carrier moieties) or different (e.g., a liphilic carrier moiety and a peptidyl carrier moiety). Attachment of multiple carrier moieties on a compound of the present invention may be identical (e.g., both covalently attached) or different (e.g., one covalently attached and one non-covalently attached).

The blood-brain barrier is a permeability barrier that exists between the extracellular fluid in the brain and the blood in the capillary lumen. The barrier stems from structural differences between the capillaries in the brain and capillaries found in other tissues. Most significant among the structural differences of brain capillaries are the tight junctions between endothelial cells. These specialized tight junctions create a very high trans-endothelial electrical resistance of 1500-2000 ohms/cm² compared to 3-33 ohms/cm² in capillary endothelial cells lying outside the brain, reducing the aqueous based para-cellular diffusion observed in other organs (Brightman, M. in Bradbury MWB (ed.) Physiology and Pharmacology of the blood-brain barrier. Handbook of experimental pharmacology 103, Springer-Verlag, Berlin, (1992); Lo, E. H., et al., Brain Res. Rev., 38:140-148, (2001)). Thus, in some embodiments, the compounds of the present invention are covalently attached to a carrier moiety (represented by the symbol Y in the formulae above).

Any appropriate carrier moiety may be used in the present invention. Useful carrier moieties include, for example, lipophilic carrier moieties, enzymatic substrate carrier moieties, peptidyl carrier moieties, and nanoparticle carrier moieties. Carrier moieties may also include an oligosaccharide unit or other molecule linked to the compound by phosphoester or lipid-ester or other hydrolyzable bonds which are cleaved by glycosidases, phosphatases, esterases, lipases, or other hydrolases in the lysosomes and endosomes. The carrier moieties may contain guanidine, amino, or imidizole functional groups.

1. Lipophilic Carrier Moieties

Lipophilic carrier moieties increase the overall lipophilicity of a compound, thereby aiding in passage through the BBB. Lipophilicity can be quantified using any suitable approach known in the art. For example, the partition coefficient between octanol and water (log P_{o/w}) may be measured thereby indicating the degree of lipophilicity. In some embodiments, the lipophilic carrier moiety has a log P_{o/w} of 1.5-2.5. Lipophilic carrier moieties are widely known in the art and are discussed in detail, for example, in Lambert, D. M., Eur J Pharm Sci., 11:S15-27 (2000). Exemplary lipophilic carrier moieties used to increase the lipophilicity of a compound include modified and unmodified diglycerides, fatty acids, and phospholipids.

Some lipophilic carrier moieties undergo enzyme mediated oxidation after traversing the BBB, resulting in a hydrophilic membrane impermeable form of the carrier moiety that remains trapped behind the BBB (Bodor et al., Pharmacol Ther 76:1-27 (1997); Bodor et al., American Chemical Society, Washington, D.C. pp 317-337 (1995); Chen et al., J Med Chem 41:3773-3781 (1998); Wu et al., J Pharm Pharmacol 54:945-950 (2002)). Exemplary lipophilic carrier moieties that undergo enzyme mediated oxidation include 1,4-dihydrotrigonelline (Palomino et al., J Med Chem, 32:622-625 (1989)); alkyl phosphonate carrier moieties that have been successfully used to transport testosterone and zidovudine across the blood-brain barrier (Somogyi, G., et al., Int J Pharm, 166:15-26 (1998)); and the lipophilic dihydropyridine carrier moieties that are enzymatically oxidized to the ionic pyridinium salt (Bodor et al., Science, 214(18):1370-1372 (1981)).

2. Peptidyl Carrier Moieties

Peptidyl carrier moieties are moieties partially or wholly composed of a peptide (including polypeptides, proteins, antibodies, and antibody fragments) used to aid in the transport of compounds across the BBB (Wu et al., J Clin Invest 100:1804-1812 (1997); U.S. Pat. No. 4,801,575; Pardridge et al., Adv Drug Deliv Rev, 36:299-321 (1999)).

Peptidyl carrier moieties may interact with specific peptide transport systems, receptors, or ligands that target the corresponding ligand or receptor on an endothelial cell of the BBB. Specific transport systems may include either carrier-mediated or receptor-mediated transport across the BBB (U.S. Pat. App. No. 20040110928). Exemplary peptidyl carrier moieties include insulin (Pardridge et al., Nat Rev Drug Discov, 1:131-139 (2002)); small peptides such as enkephalin, thyrotropin-releasing hormone, arginine-vassopressin (Bergley, J Pharm Pharmacol, 48:136-146 (1996)), Banks et al., Peptides, 13:1289-1294 (1992)), Han et al., AAPS Pharm. Si., 2:E6 (2000)); chimeric peptides such as those described in WO-A-89/10134; amino acid derivatives such as those disclosed in U.S. Pat. App. No. 20030216589; tat peptide (Schwarze, S. R., et al., Science 285:1569-1572 (1999); polyarginine peptide (Wender, P. A., et al., Proc. Natl. Acad. Sci. USA 97:13003-13008 (2000)); insulin-like-growth factor-1; insulin-like-growth factor-2; transferrin; leptin; low-density lipoprotein (Pardridge, Nat. Rev. Drug Discov. 1:131-139 (2002); Colma et al., Pharm. Res. 17:266-274 (2000); Pardridge, Endocrine Rev, 7:314-330 (1986); Golden, et al., J Clin Invest, 99:14-18 (1997); Bickel et al., Adv. Drug Deliv. Rev. 46(1-3):247-79 (2001)); and basic fibroblast growth factor (bFGF) (U.S. Pat. App. No. 20040102369).

U.S. Application No. 20040121947, and International Application No. PCT/US02/34324 (Publication No. WO 03/039454), disclose that confocal microscopic images of cells incubated with a fluorescent tat-conjugated isosteric β-secretase inhibitor showed uneven distribution inside cells. Some high fluorescence intensity was associated with the endosome and lysosome intracellular vesicular structures. This indicated that the tat carrier moiety may have been modified by proteases within the lysosome or endosome resulting in an inhibitor that was unable to exit the lysosomal or endosomal compartment. Lysosomes and endosomes contain many proteases, including hydrolase such as cathepsins A, B, C, D, H and L. Some of these are endopeptidase, such as cathepsins D and H. Others are exopeptidases, such as cathepsins A and C, with cathepsin B capable of both endo- and exopeptidase activity. The specificities of these proteases are sufficiently broad to hydrolyze a tat peptide away from the inhibitor compound, thus, hydrolyzing the carrier peptide away from the isosteric inhibitor. Thus, it has been shown that tat and other carrier peptides may be particularly useful for specific delivery of isosteric inhibitors to lysosomes and endosomes. When administered to a mammal by a mechanism such as injections, the conjugated compound will penetrate cells and permeate to the interior of lysosomes and endosomes. The proteases in lysosomes and endosomes will then hydrolyze tat, thereby preventing to escape from lysosomes and endosomes.

The carrier peptide may be tat or other basic peptides, such as oligo-L-arginine, that are hydrolyzable by lysosomal and endosomal proteases. Specific peptide bonds susceptible for the cleavage of lysosomal or endosomal proteases may be installed, thereby facilitating the removal of the carrier compound from the inhibitor. For example, dipeptides Phe-Phe, Phe-Leu, Phe-Tyr and others are cleaved by cathepsin D.

In one embodiment, the peptidyl carrier molecule includes cationic functional groups, such as the tat-peptide (Schwarze, S. R., et al., Science 285: 1569-1572 (1999)), or nine arginine residues (Wender, P. A., et al., Proc. Natl. Acad. Sci. USA 97:13003-13008 (2000)). Useful cationic functional groups include, for example, guanidine, amino, and imidazole functional groups. Thus, cationic functional groups also include amino acid side chains such as side chains of lysine, arginine, and histidine residues. In some embodiments, the peptidyl carrier molecule may include from 1-10 cationic functional groups. When a compound of the invention is conjugated or attached to a carrier moiety, the resulting conjugate may be referred to herein as a “Carrier Peptide-Inhibitor” conjugate or “CPI.” The CPI conjugate can be administered to an in vitro sample or to a mammal thereby serving as a transport vehicle for a compound or compounds of the invention into a cell in an in vitro sample or in a mammal. The carrier moieties and CPI conjugates result in an increase in the ability of the compounds of the invention to effectively penetrate cells and the blood brain barrier to inhibit memapsin 2 from cleaving APP to subsequently generate Aβ.

Adsorptive-meditated transcytosis (AME) provides an alternative mechanism whereby peptidyl carrier moieties may cross the BBB. AME differs from other forms of transcytosis in that the initial binding of the carrier moiety to the luminal plasma membrane is mediated through either electrostatic interactions with anionic sites, or specific interactions with sugar residues. Uptake through AME is determined by the C-terminal structure and basicity of the carrier moiety. Exemplary adsorptive peptidyl carrier moieties include peptides and proteins with basic isoeletric points (cationic proteins), and some lectins (glycoprotein binding proteins). See Tamai, I., et al., J. Pharmacol. Exp. Ther. 280:410-415 (1997); Kumagai, A. K., et al., J. Biol. Chem. 262: 15214-15219 (1987).

Peptidyl carrier moieties also include antibody carrier moieties. Antibody carrier moieties are carrier moieties that include an antibody or fragment thereof. Typically, the antibody or antibody fragment is, or is derived from, a monoclonal antibody. Antibody carrier moieties bind to cellular receptors, or transporters expressed on the luminal surface of brain capillary endothelial cells (U.S. Patent App No. 20040101904). Exemplary antibodies, or fragments thereof, include MAb 83-14 that binds to the human insulin receptor (Pardridge et al., Pharm Res. 12:807-816 (1995)); anti-transferrin antibody (Li, J. Y., et al., Protein Engineering 12:787-796 (1999)); and monoclonal antibodies that mimic an endogenous protein or peptide which is known to cross the BBB as discussed above.

3. Nanoparticle Carrier Moieties

Nanoparticle carrier moieties are solid colloidal carriers generally less than a micron in diameter or length. The compound may be encapsulated in, adsorbed onto, or covalently linked to the surface of the nanoparticle carrier moiety. Nanoparticle carrier moieties have been used to successfully deliver a variety of compounds to the brain, including hexapeptide dalagrin, an enkephalin analog; loperamide; tubocerarine; and doxorubicin (Ambikanandan, et al., J. Pharm Pharmaceut Sci 6(2):252-273 (2003)). In addition to aiding transport into the brain, nonionic detergents such as polysorbate-80, which can be used to coat the nanoparticle, may be used to inhibit the efflux pump. Zordan-Nudo, T., et al., Cancer Res, 53:5994-6000 (1993). Exemplary materials for the manufacture of nanoparticle carrier moieties include polyalkylcyanoacrylate (PACA) (Bertling et al., Biotechnol. Appl. Biochem. 13: 390-405 (1991)); polybutylcyanoacrylate (PBCA) (Chavany et al., Pharm. Res. 9: 441-449 (1992)); polybutylcyanoacrylate with the peptide-drug complex absorbed onto the surface and coated with polysorbate 80 (Kreuter, J., et al., Brain Res, 674:171-174 (1995), Kreuter, J. Adv Drug Deliv Rev, 47:65-81, (2001), Kreuter, J. Curr Med Chem, 2:241-249 (2002)); polyisohexylcyanoacrylate (PIHCA) (Chavany et al., Pharm. Res. 11: 1370-1378 (1994)); polyhexylcyanoacrylate (PHCA) (Zobel et al., Antisense Nucleic Acid Drug Dev. 7:483-493 (1997)); and PEGylated polycyanoacrylate (Pilar, C., et al., Pharm Res 18(8):1157-1166 (2001)).

4. Linker Moieties

Linker moieties may be used to attach the carrier moiety to the β-secretase inhibitors of the present invention. For example, steric hinderance between the compound and the carrier can be prevented using polymer technology (e.g., PEGylation) in conjunction with the linker molecule to introduce a long spacer arm (Yoshikawa, T., et al., J Pharmacol Exp Ther, 263:897-903, 1992). Linker moieties may be cleavable or non-cleavable.

Cleavable linker molecules include a cleavable moiety. Any appropriate cleavable moiety is useful in the present invention, including for example, phosphoesters, esters, disulfides, and the like. Cleavable moieties also include those moieties capable of being cleaved by biological enzymes, such as peptidases, glycosidases, phosphatases, esterases, lipases, or other hydrolases. Cleavable linker molecules are especially useful where the carrier moiety interferes with the biological activity of the compound. Exemplary cleavable linker molecules include N-succinimidyl-3-2-pyridyldithioproprionate (SPDP), or N-hydrosuccinimide (NHS).

Non-cleavable linker molecules are those that involve the attachment of a carrier moiety to the compound through a linkage that is generally stable to biological conditions and enzymes. Non-cleavable linker molecules are typically used when the carrier moiety does not interfere with the biological activity of the compound. Exemplary non-cleavable linker molecules include thio-ether (e.g., m-maleimidobenzoyl N-hydroxysuccinimide ester (MBS)); amide (e.g., N-hydrosuccinimide (NHS-XX-); extended amide (e.g., N-hydrosuccinimide polyethylene glycol (NHS-PEG); and extended hydrazide linkages (e.g., hydrazide-PEG-biotin-); avidin-biotin; and PEG linkers (Ambikanandan et al., J. Pharm Pharmaceut Sci 6(2):252-273 (2003); Pardridge, Adv Drug Deliv Rev, 36:299-321 (1999); U.S. Pat. No. 6,287,792).

### II. GENERAL SYNTHETIC METHODS

The compounds of the invention are synthesized by an appropriate combination of generally well-known synthetic methods. Techniques useful in synthesizing the compounds of the invention are both readily apparent and accessible to those of skill in the relevant art. The discussion below is offered to illustrate certain of the diverse methods available for use in assembling the compounds of the invention. However, the discussion is not intended to define the scope of reactions or reaction sequences that are useful in preparing the compounds of the present invention.

A method for synthesizing compounds of the invention is by adapting the synthesis for N¹-((2S,3R)-3-hydroxy-4-((3-methoxybenzyl)(methyl)amino)-1-phenylbutan-2-yl)-N³-methyl-N³-((4-methylthiazol-2-yl)methyl)isophthalamide (1):

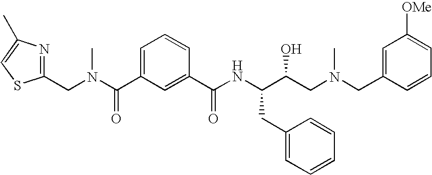

which is shown below in Scheme 1:

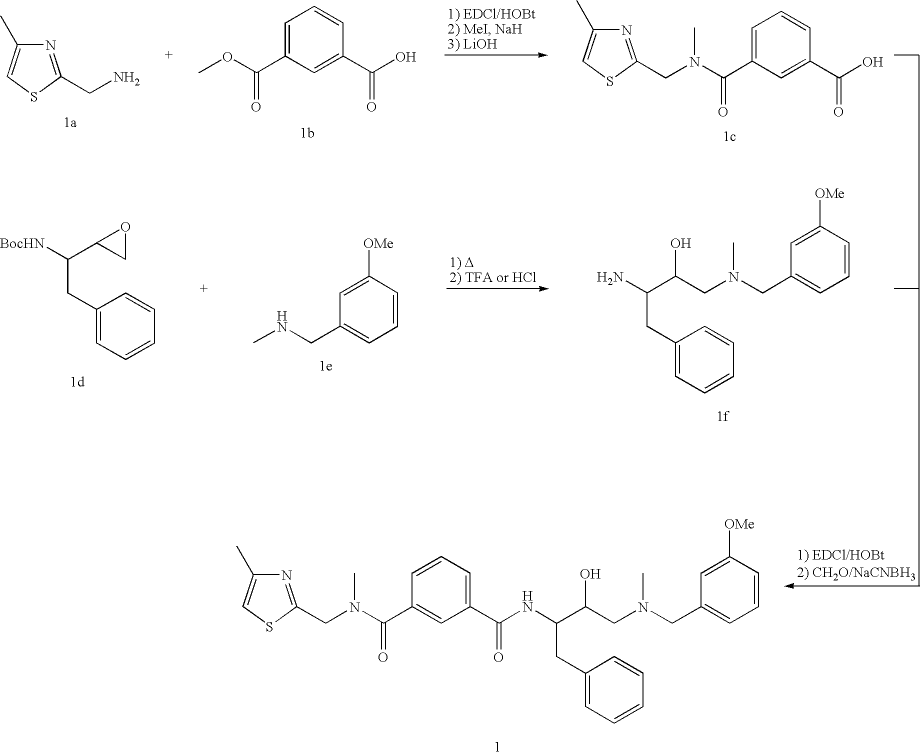

Synthesis of amine 1a and partially protected isophthalic acid 1b (and related building blocks for variations of the invention) are detailed in the Examples section below. The corresponding isophthalamides may be formed, for example, by coupling an amine 1a with the partially protected isophthalic acid 1b using a coupling agent, such as 1-ethyl-3-(3-dimethylaminopropyl)carbodiimide hydrochloride (EDCI) with 1-hydroxybenzotriazole (HOBT). Alkylation of the resulting amide may be carried out using sodium hydride and an alkyl halide. Alternatively, the amide coupling may be carried out using a secondary amine to generate the alkylated amide. Ester hydrolysis under basic conditions (for example, using LiOH) may be used to generate the desired carboxylate fragment 1c.

Treatment of the Boc-protected epoxide 1d with an appropriate amine 1e, followed by removal of the Boc protecting group under acidic conditions yields aminoalcohol building block 1f.

Alternatively, as shown in the examples below, epoxides such as 1d may be coupled to an appropriate aldehyde to generate the corresponding amino alcohol. Here the epoxide ring-opening is conducted using ammonia to generate a primary amine which is subsequently coupled to the desired aldehyde under reductive amination conditions (such as NaBH₃CN or NaB(OAc)₃H, followed by acid) to generate the desired fragment.

Standard amide coupling of 1f with fragment 1c using common coupling agents (e.g., EDCI with HOBt; CDI; DCC; DCI; etc.) may be used to generate the desired inhibitors, such as 1.

### III. Beta-Secretase Inhibitor Activity

To develop useful β-secretase inhibitors, candidate inhibitors capable of selectively decreasing memapsin 2 catalytic activity may be identified in vitro and subsequently tested for their ability to reduce the production of Aβ. The activity of the inhibitor compounds can be assayed utilizing methods known in the art and/or those methods presented herein.

Compounds that decrease memapsin 2 catalytic activity may be identified and tested using biologically active memapsin 2, either recombinant or naturally occurring. Memapsin 2 can be found in native cells, isolated in vitro, or co-expressed or expressed in a cell. Measuring the reduction in the memapsin 2 catalytic activity in the presence of an inhibitor relative to the activity in the absence of the inhibitor may be performed using a variety of methods known in the art.

For example, the compounds may be tested for their ability to cause a detectable decrease in hydrolysis of a β-secretase site of a peptide in the presence of memapsin 2. These data can be expressed, for example, as Kᵢ, Kᵢ apparent, Vᵢ/Vₒ, or percentage inhibition. Kᵢ is the inhibition equilibrium constant which indicates the ability of compounds to inhibit a given enzyme (such as memapsin 2, memapsin 1, and/or cathepsin D). Numerically lower Kᵢ values indicate a higher affinity of the compounds of the invention for the enzyme. The Kᵢ value is independent of the substrate, and converted from Kᵢ apparent.

Kᵢ apparent is determined in the presence of substrate according to established techniques (see, for example, Bieth, J., Bayer-Symposium V: Proteinase Inhibitors, pp. 463-469, Springer-Verlag, Berlin (1994)). The standard error for the Kᵢ apparent is the error from the nonlinear regression of the Vᵢ/Vₒ data measured at different concentrations of the compounds of the invention (e.g., between about 10 nM to about 1000 nM) employing well-known techniques (see, for example, Bieth, J., Bayer-Symposium V: Proteinase Inhibitors, pp. 463-469, Springer-Verlag, Berlin (1994), Ermolieff, J., et al., Biochemistry 39:12450-12456 (2000), the teachings of which are incorporated herein by reference in their entirety). Vᵢ/Vₒ depicts the ratio of initial conversion velocities of an enzyme substrate (Ermolieff, et al., Biochemistry 40:12450-12456 (2000)) by an enzyme in the absence (Vₒ) or presence (Vᵢ) of an inhibitor. A Vᵢ/Vₒ value of 1.0 indicates that a compound does not inhibit the enzyme at the concentration tested. A Vᵢ/Vₒ value less than 1.0 indicates that a compound of the invention inhibits enzyme activity.

In some embodiments, the compounds described herein (e.g., any compound or group of compounds of Example 3) are capable of reducing memapsin 2 beta-secretase activity. In some embodiments, the compounds have a memapsin 2 beta-secretase Kᵢ and/or Kᵢ apparent (e.g., using any inhibitory assay described herein) of less than about 10 μM, 5 μM, 1 μM, or less than about 750, 500, 400, 300, 200, 100, 50, 25, 10, 5, 2, or 1 nM; or from about 1 to 5, 1 to 10, 1 to 100, 1 to 300, 1 to 500, 1 to 1000, 100 to 500, 200 to 500, 300 to 500, 100 to 750, 200 to 750, 300 to 750, 400 to 750, 500 to 750, 100 to 1000, 250 to 1000, 500 to 1000, or 750 to 1000 nM. In some embodiments, the compounds have a memapsin 2 beta-secretase Kᵢ and/or Kᵢ apparent (e.g., using any inhibitory assay described herein) of less than 300, 301 to 500, or greater than 501 nM.

Once compounds are identified that are capable of reducing the hydrolysis of a β-secretase site of a peptide in the presence of memapsin 2, the compounds may be further tested for their ability to selectively inhibit memapsin 2 relative to other enzymes. Typically, the other enzyme is a peptide hydrolase, such as memapsin 1 or cathepsin D. Compounds that decrease cathepsin D catalytic activity or memapsin 1 catalytic activity are tested using biologically active enzyme, either recombinant or naturally occurring. Cathepsin D or memapsin 1 catalytic activity can be found in native cells, isolated in vitro, or co-expressed or expressed in a cell. Inhibition by a compound of the invention is measured using standard in vitro or in vivo assays such as those well known in the art or as otherwise described herein.

For example, selectivity of a compound may be measured by determining the extent to which memapsin 2 hydrolyzes a substrate peptide in the presence of the compound compared to the extent to which the same compound inhibits memapsin 1 and/or cathepsin D cleaving of a β-secretase site of a substrate peptide. Exemplary substrate peptides are useful in determining the activity of memapsin 2 includes APP and derivatives thereof, such as FS-2 (MCA-SEVNLDAEFR-DNP; SEQ ID NO.: 2) (Bachem Americas, Torrance, Calif.). Exemplary substrate peptides are useful in determining the activity of memapsin 1 and cathepsin D include, for example, peptides which include the sequence ELDLAVEFWHDR (SEQ ID NO.: 1). These substrate peptides can be synthesized using known peptide synthesis methods, e.g., solid-phase peptide synthesis (e.g., FMOC amino acid coupling etc.). These data can be expressed, for example, as Kᵢ, Kᵢ apparent, Vᵢ/Vₒ, or percentage inhibition and depict the inhibition of a compound for memapsin 2 catalytic activity relative to memapsin 1 or cathepsin D catalytic activity. For example, if the Kᵢ of a reaction between an inhibitor compound of the invention and memapsin 1 or cathepsin D is 1000 and the Kᵢ of a reaction between an inhibitor compound of the invention and memapsin 2 is 100, the inhibitor compound inhibits the β-secretase activity of memapsin 2 with ten-fold selectivity over memapsin 1 or cathepsin D.

In some embodiments, the compounds described herein (e.g., any compound or group of compounds of Example 3) are capable of selectively reducing memapsin 2 relative to memapsin 1 and/or cathepsin D. In some embodiments, the compounds are capable of selectively reducing memapsin 2 relative to memapsin 1 and/or cathepsin D with greater than about 2-fold selectivity, or greater than about 3, 5, 7, 10, 25, 50, 75, 100, 300, 200, 500, 750, 1000, 2000, 5000, or 10000-fold selectivity. In some embodiments, the compounds have a memapsin 2 beta-secretase Kᵢ and/or Kᵢ apparent (e.g., using any inhibitory assay described herein) of less than about 10 μM, 5 μM, 1 μM, or less than about 750, 500, 400, 300, 200, 100, 50, 25, 10, 5, 2, or 1 nM, or from about 1 to 5, 1 to 10, 1 to 100, 1 to 300, 1 to 500, 1 to 1000, 100 to 500, 200 to 500, 300 to 500, 100 to 750, 200 to 750, 300 to 750, 400 to 750, 500 to 750, 100 to 1000, 250 to 1000, 500 to 1000, or 750 to 1000 nM; and have a memapsin 1 and/or cathepsin D Kᵢ and/or Kᵢ apparent of more than about 10 μM, 5 μM, 1 μM, or more than about 750, 500, 400, 300, 200, 100, 50, 25, 10, 5, 2, or 1 nM, or from about 1 to 5, 1 to 10, 1 to 100, 1 to 300, 1 to 500, 1 to 1000, 100 to 500, 200 to 500, 300 to 500, 100 to 750, 200 to 750, 300 to 750, 400 to 750, 500 to 750, 100 to 1000, 250 to 1000, 500 to 1000, or 750 to 1000 nM.

Compounds demonstrating the ability to cause a detectable decrease in hydrolysis of a β-secretase site of a peptide in the presence of memapsin 2 (or, in addition, selectivity of action toward memapsin 2), may be tested in cell models or animal models for their ability to cause a detectable decrease in the amount or production of β-amyloid protein (Aβ). For example, isosteric inhibitors of memapsin 2 have been tested for their ability to decrease Aβ production in cultured cells (see U.S. Patent Application Publication No. 20040121947, International Application No. PCT/US02/34324 (Publication No. WO 03/039454), and International Application No. PCT/US06/13342 (Publication No. WO 06/110668, the contents of which are hereby incorporated by reference)). Briefly, inhibitors may be added to a culture of cells (e.g., human embryonic kidney (HEK293) cells, HeLa cells, Chinese hamster ovary cells, or neuroblastoma line M17 cells) stably transfected with a nucleic acid constructs that encode human APP Swedish mutant (or London mutation or double mutant) and, if needed, a nucleic acid construct encoding human memapsin 2. Immunoprecipitation of Aβ followed by SDS-gel electrophoresis allows detection and quantitation of the amount of Aβ produced in the presence and absence of inhibitor.

In addition to cell cultures, animal models may be used to test inhibitors of memapsin 2 for their ability to decrease Aβ production. For example, an animal (e.g., tg2576 mice) expressing the Swedish mutation of the human amyloid precursor protein (Hsiao, K., et al., Science 274, 99-102 (1996) may be injected intraperitoneally with an inhibitor. The plasma may then be collected and Aβ levels determined by capture ELISA (BioSource International, Camarillo, Calif.).

In some embodiments, the compounds described herein (e.g., any compound or group of compounds of Example 3) are capable of reducing cellular Aβ production. In some embodiments, the compounds are capable of reducing cellular Aβ production with a IC50 (e.g., using an Aβ inhibitory assay described herein) of less than about 10 μM, 5 μM, 1 μM, or less than about 750, 500, 400, 300, 200, 100, 50, 25, 10, 5, 2, or 1 nM, or from about 1 to 5, 1 to 10, 1 to 100, 1 to 300, 1 to 500, 1 to 1000, 100 to 500, 200 to 500, 300 to 500, 100 to 750, 200 to 750, 300 to 750, 400 to 750, 500 to 750, 100 to 1000, 250 to 1000, 500 to 1000, or 750 to 1000 nM. In some embodiments, the compounds are capable of reducing cellular Aβ production with a IC50 (e.g., using an Aβ inhibitory assay described herein) of less than 1 μM, between 1 and 5 μM, or greater than 5 μM.

The presence of inhibitors in organs of animal models or within cellular compartments may be ascertained using a fluorescent tag conjugated to the inhibitor and visualization via confocal microscopy (see U.S. Patent Application Publication No. 20040121947, and International Application No. PCT/US02/34324 (Publication No. WO 03/039454), the contents of which are hereby incorporated by reference in its entirety).

The sample obtained from the mammal can be a fluid sample, such as a plasma or serum sample; or can be a tissue sample, such as a brain biopsy. The amount of β-secret protein or a decrease in the production of β-secret protein can be measured using standard techniques (e.g., western blotting and ELISA assays).

Further examples of assays for identifying memapsin 2-β-secretase inhibitors are set forth in the Examples section below. Other methods for assaying the activity of memapsin 2, memapsin 1, and cathepsin D and the activity of agents that decrease the activity of these enzymes are known in the art. The selection of appropriate assay methods is well within the capabilities of those of skill in the art, particularly in view of the teaching provided herein.

### IV. PHARMACEUTICAL COMPOSITIONS

In another aspect, the present invention provides pharmaceutical compositions comprising a memapsin 2 β-secretase inhibitor compound of the invention or a memapsin 2 β-secretase inhibitor compound in combination with a pharmaceutically acceptable carrier. The pharmaceutical compositions may include optical isomers, diastereomers, or pharmaceutically acceptable salts of the inhibitors disclosed herein. The memapsin 2 β-secretase inhibitor included in the pharmaceutical composition may be covalently attached to a carrier moiety, as described above. Alternatively, the memapsin 2 β-secretase inhibitor included in the pharmaceutical composition is not covalently linked to a carrier moiety.

Certain inhibitor compounds described herein may have exceptional pharmacokinetic properties (e.g., brain penetration). For example, N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide was determined to have 46.8% brain penetration relative to plasma after 24 hours of administration and a maximum brain concentration from a 10 mg/kg i.v. dosage of 160 ng/mL at 15 min (see e.g., Example 7 herein). Accordingly, in one aspect, the inhibitor compounds of the present invention are capable of penetrating the brain (e.g., can be detected in the tissue of the brain after administration to an individual (e.g., a test subject (e.g., rat, monkey, dog or other suitable non-human test subject)). In some of these embodiments, the inhibitor compound is capable of greater than about 25%, 30%, 35%, 40%, 45%, 50%, 55%, 60%, 65%, or 70% brain penetration in an individual, for example, a test subject (e.g., a non-human test subject (e.g., monkey, dog, rat, etc.)) as a peak value and/or a defined period following administration (e.g., 12, 24, 36 hrs) relative to plasma, such as measured by methods described in Example 7. In some embodiments, the maximum concentration of the inhibitor compound in the brain of an individual (e.g., a test subject, (e.g., a non-human test subject (e.g., monkey, dog, rat, etc.))) from an i.v. administered dosage of about 10 mg/kg (or about 5 mg/kg, or about 50 mg/kg) is greater than about 100 ng/mL, or about 115, 130, 145, 160, 175, 200, 225, 250, 300, 500, or 1000 ng/mL, or between about 1 and 2000 ng/mL, or about 10 and 1000, 50 and 500, 100 and 250, or 150 and 200 ng/mL, such as measured by methods described in Example 7. Modifications of the assay methods described herein for determination of preferential penetration of the brain (e.g., measurement of compound present in brain tissue) by compounds described herein will be apparent to the skilled artisan in view of the teaching provided herein.

A “pharmaceutically suitable carrier,” as used herein refers to pharmaceutical excipients, for example, pharmaceutically, physiologically, acceptable organic, or inorganic carrier substances suitable for enteral or parenteral application which do not deleteriously react with the extract. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compounds of the invention.

The compounds of the invention can be administered alone or can be coadministered to the individual. Coadministration is meant to include simultaneous or sequential administration of the compounds individually or in combination (more than one compound). Thus, the preparations can also be combined, when desired, with other active substances related to the treatment of a specified condition (e.g., to reduce metabolic degradation).

A. Formulations

The β-secretase inhibitors of the present invention can be prepared and administered in a wide variety of oral, parenteral and topical dosage forms. Thus, the compounds of the present invention can be administered by injection (e.g., intravenously, intramuscularly, intracutaneously, subcutaneously, intraduodenally, or intraperitoneally). Also, the compounds described herein can be administered by inhalation, for example, intranasally. Additionally, the compounds of the present invention can be administered transdermally. Compounds of the invention may also be administered locally (e.g., ocular administration such as topical eye drops or ointment). It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of both of which are hereby incorporated by reference.

Ocular administration preparations (e.g., in use of glaucoma treatment) include, but are not limited to, formulations in saline, optionally with additional carriers, stabalizers, etc. know to those of skill in the art.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

B. Effective Dosages

Pharmaceutical compositions provided by the present invention include compositions wherein the active ingredient is contained in an effective amount, i.e., in an amount effective to achieve its intended purpose. The actual amount effective for a particular application will depend, inter alia, on the condition being treated. For example, when administered in methods to treat Alzheimer's disease, such compositions will contain an amount of active ingredient effective to achieve the desired result (e.g., decreasing β-secretase activity or β-amyloid production). Determination of an effective amount of a compound of the invention is well within the capabilities of those skilled in the art, especially in light of the detailed disclosure herein.

The dosage and frequency (single or multiple doses) administered to a mammal can vary depending upon a variety of factors, including a disease that results in increased activity of memapsin 2 or increased accumulation of β-secret protein, whether the mammal suffers from another disease, and its route of administration; size, age, sex, health, body weight, body mass index, and diet of the recipient; nature and extent of symptoms of the disease being treated (e.g., Alzheimer's disease), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the effective amount can be initially determined from cell culture assays. Target concentrations will be those concentrations of active compound(s) that are capable of reducing the activity of memapsin 2 activity, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring memapsin 2 inhibition and adjusting the dosage upwards or downwards, as described above. Adjusting the dose to achieve maximal efficacy in humans based on the methods described above and other methods as are well-known in the art is well within the capabilities of the ordinarily skilled artisan, particularly in view of the teaching provided herein.

Dosages may be varied depending upon the requirements of the individual and the compound being employed. The dose administered to an individual, in the context of the present invention should be sufficient to affect a beneficial therapeutic response in the individual over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment of the invention, the dosage range is 0.001% to 10% w/v. In another embodiment, the dosage range is 0.1% to 5% w/v.

Additional examples of dosages which can be used are an effective amount within the dosage range of about 0.1 μg/kg to about 300 mg/kg, or within about 1.0 μg/kg to about 40 mg/kg body weight, or within about 1.0 μg/kg to about 20 mg/kg body weight, or within about 1.0 μg/kg to about 10 mg/kg body weight, or within about 10.0 μg/kg to about 10 mg/kg body weight, or within about 100 μg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight. Compounds of the present invention may be administered in a single daily dose, or the total daily dosage may be administered in divided dosage of two, three or four times daily.

Utilizing the teachings provided herein, an effective prophylactic or therapeutic treatment regimen can be planned which does not cause substantial toxicity and yet is entirely effective to treat the clinical symptoms demonstrated by the particular individual. This planning should involve the careful choice of active compound by considering factors such as compound potency, relative bioavailability, individual body weight, presence and severity of adverse side effects, preferred mode of administration and the toxicity profile of the selected agent.

C. Kits

Also provided are kits for administration of the compositions described herein (e.g., including the compounds, formulations, and dosage forms described herein).

In certain embodiments the kits may include a dosage amount of at least one composition as disclosed herein. Kits may further comprise suitable packaging and/or instructions for use of the composition. Kits may also comprise a means for the delivery of the composition thereof.

The kits may include other pharmaceutical agents for use in conjunction with the composition described herein. In some variations, the pharmaceutical agent(s) may be one or more anti-psychotic drug. These agents may be provided in a separate form, or mixed with the compounds of the present invention, provided such mixing does not reduce the effectiveness of either the pharmaceutical agent or composition described herein and is compatible with the route of administration. Similarly the kits may include additional agents for adjunctive therapy or other agents known to the skilled artisan as effective in the treatment or prevention of the conditions described herein.

The kits may optionally include appropriate instructions for preparation and administration of the composition, side effects of the composition, and any other relevant information. The instructions may be in any suitable format, including, but not limited to, printed matter, videotape, computer readable disk, optical disc or directions to internet-based instructions.

In another aspect of the invention, kits for treating an individual who suffers from or is susceptible to the conditions described herein are provided, comprising a first container comprising a dosage amount of a composition as disclosed herein, and instructions for use. The container may be any of those known in the art and appropriate for storage and delivery of intravenous composition. In certain embodiments the kit further comprises a second container comprising a pharmaceutically acceptable carrier, diluent, adjuvant, etc. for preparation of the composition to be administered to the individual.

Kits may also be provided that contain sufficient dosages of the inhibitor (including compositions thereof) as disclosed herein to provide effective treatment for an individual for an extended period, such as 1-3 days, 1-5 days, a week, 2 weeks, 3, weeks, 4 weeks, 6 weeks, 8 weeks, 3 months, 4 months, 5 months, 6 months, 7 months, 8 months, 9 months or more.

Kits may also include multiple doses of the composition and instructions for use and packaged in quantities sufficient for storage and use in pharmacies, for example, hospital pharmacies and compounding pharmacies.

The kits may include the composition as described herein packaged in either a unit dosage form or in a multi-use form. The kits may also include multiple units of the unit dose form.

In certain embodiments, are provided the composition described herein in a unit dose form. In other embodiments the compositions may be provided in a multi-dose form (e.g., a blister pack, etc.).

D. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g., Fingl et al., In: THE PHARMACOLOGICAL BASIS OF THERAPEUTICS, Ch. 1, p. 1, 1975. The exact formulation, route of administration and dosage can be chosen by the individual physician in view of the individual's condition and the particular method in which the compound is used.

### V. METHODS OF REDUCING THE ACTIVITY OF MEMAPSIN 2 BETA-SECRETASE

In another aspect of the present invention, the β-secretase inhibitor compounds of the invention can be employed in methods to decrease memapsin 2 activity, decrease hydrolysis of a β-secretase site of a memapsin 2 substrate, and/or decrease the accumulation of β-amyloid protein relative to the amount of memapsin 2 activity, hydrolysis of a β-secretasesite, and accumulation of β-secret protein, respectively, in the absence of the β-secretase inhibitor.

In an exemplary embodiment, a method of reducing memapsin 2 activity is provided. The method includes contacting a memapsin 2 with a β-secretase inhibitor compound of the present invention. The memapsin 2 may be contacted in any appropriate environment (e.g., in vitro, ex vivo, in vivo). The memapsin 2 activity is decreased relative the amount of activity in the absence of β-secretase inhibitor.

In another exemplary embodiment, a method is provided of selectively reducing memapsin 2 activity using an inhibitor of the present invention. Selective reduction of the activity of memapsin 2 means that memapsin 2 is not only reduced relative to its activity in the absence of inhibitor, but is reduced to a greater extent as compared to the reduction in activity due to inhibitor action against another peptide hydrolase. For example, as described above, the reduction in activity of an enzyme may be expressed in terms of the inhibitory constant (Kᵢ). Where an inhibitor selectively reduces the activity of memapsin 2, the Kᵢ of the reaction between an inhibitor compound of the invention and memapsin 2 is less than the Kᵢ of the reaction between an inhibitor compound of the invention and another peptide hydrolase.

In an exemplary embodiment, the Kᵢ of the reaction between an inhibitor compound of the invention and memapsin 2 is at least 2 times less than the Kᵢ of the reaction between an inhibitor compound of the invention and another peptide hydrolase. In another exemplary embodiment, the Kᵢ of the reaction between an inhibitor compound of the invention and memapsin 2 is at least 10 times less than the Kᵢ of the reaction between an inhibitor compound of the invention and another peptide hydrolase. In another exemplary embodiment, the Kᵢ of the reaction between an inhibitor compound of the invention and memapsin 2 is at least 100 times less than the Kᵢ of the reaction between an inhibitor compound of the invention and another peptide hydrolase. In another exemplary embodiment, the Kᵢ of the reaction between an inhibitor compound of the invention and memapsin 2 is at least 1000 times less than the Kᵢ of the reaction between an inhibitor compound of the invention and another peptide hydrolase. In another exemplary embodiment, the Kᵢ of the reaction between an inhibitor compound of the invention and memapsin 2 is at least 10000 times less than the Kᵢ of the reaction between an inhibitor compound of the invention and another peptide hydrolase.

In some related embodiments, the inhibitor selectively reduces the activity of memapsin 2 as compared to memapsin 1. In other related embodiments, the inhibitor selectively reduces the activity of memapsin 2 as compared to cathepsin D.

Thus, the present invention provides methods of selectively reducing the activity of memapsin 2. The method includes contacting a memapsin 2 with a β-secretase inhibitor compound of the present invention. In a related embodiment, the method includes contacting the memapsin 2 with a β-secretase inhibitor in the presence of memapsin 1. In an alternative related embodiment, the method includes contacting the memapsin 2 with a β-secretase inhibitor in the presence of cathepsin D. In yet another related embodiment, the method includes contacting the memapsin 2 with a β-secretase inhibitor in the presence of cathepsin D and memapsin 1.

In some embodiments, the activity of memapsin-2 β-secretase may be determined by measuring the hydrolysis of a β-secretase site of a β-secretase substrate. Thus, the present invention also relates to a method of decreasing the hydrolysis of a β-secretase site of a β-secretase substrate by contacting a memapsin 2 with a β-secretase inhibitor compound of the present invention. In some embodiments, the hydrolysis of a β-secretase site is decreased relative the amount of hydrolysis in the absence of the inhibitor. In other embodiments, the hydrolysis is selectively reduced as compared to hydrolysis by memapsin 1 and/or cathepsin D. Thus, a method of selectively decreasing hydrolysis of a β-secretase site of a β-secret precursor protein relative to memapsin 1 and/or cathepsin D in a sample is provided. The method includes contacting a memapsin 2 with a β-secretase inhibitor compound of the present invention.

In another embodiment, the present invention relates to a method of decreasing the amount of β-amyloid protein in a sample by contacting the memapsin 2 with an inhibitor compound of the present invention. The amount of β-amyloid protein in a sample is decreased relative the amount of β-amyloid protein in the sample in the absence of the inhibitor. Thus, the accumulation of β-amyloid protein is thereby decreased.

Memapsin 2 may be contacted in any suitable environment or any suitable sample. For example, memapsin 2 may be contacted in vitro, within a cell, or within a mammal. Typically, in vitro solutions are selected such that the components do not substantially interfere with the enzymatic activity of memapsin 2 (e.g., aqueous solutions). In some embodiments, the in vitro solution includes a biological sample, such as a mammalian sample. Exemplary mammalian samples include plasma or serum samples and tissue samples, such as a brain biopsy. Any appropriate cell or cellular sample may be selected in which to contact the memapsin 2 with the inhibitor. The cell may contain endogenous memapsin 2 or recombinant memapsin 2 as previously described (see U.S. Patent Application Publication No. 20040121947 (the contents of which are hereby incorporated by reference), and International Application No. PCT/US02/34324 (Publication No. WO 03/039454)). Exemplary cells include human embryonic kidney (HEK293) cells, HeLa cells, Chinese hamster ovary cells, or neuroblastoma line M17 cells Hela cells, 293 cells. In an exemplary embodiment, the compounds of the invention are administered to a mammal to inhibit the hydrolysis of a β-secretase site of a β-amyloid precursor protein (e.g., a mouse, rabbit or human).

### VI. METHODS OF TREATING ALZHEIMER'S DISEASE

In another aspect of the present invention, the β-secretase inhibitor compounds of the invention can be employed in the treatment of diseases or conditions associated with β-secretaseactivity, hydrolysis of a β-secretase site of a β-secret precursor protein, and/or β-amyloid protein accumulation. Typically, a mammal is treated for the disease or condition. In an exemplary embodiment, the disease is Alzheimer's disease.

Thus, in some embodiments, the invention provides a method of treating Alzheimer's disease in a mammal comprising the step of administering to the mammal in need thereof an effective amount of the β-secretase inhibitors of the invention. The mammals treated with the inhibitors may be human primates, nonhuman primates or non-human mammals (e.g., rodents, canines). In one embodiment, the mammal is administered a compound of the invention that reduces β-secretase activity (inhibits memapsin 1 and memapsin 2 activity). In another embodiment, the mammal is administered a compound that selectively reduces memapsin 2 activity. In a related embodiment, the compound has minimal or no effect on reducing memapsin 1 activity. Therefore, the present invention also provides a method of treating Alzheimer's disease in a subject in need thereof, the method comprising administering to the subject an effective amount of a β-secretase inhibitor compound. In an exemplary embodiment, the β-secretase inhibitor compound is part of a pharmaceutical formulation, as described above.

The inhibitor compounds of the invention can be employed in the treatment of diseases or conditions associated with β-secretase activity, which can halt, reverse or diminish the progression of the disease or condition, in particular Alzheimer's disease. In addition to compounds that decrease memapsin 2 activity, compounds that selectively reduce memapsin 2 activity are useful to treat diseases or conditions or biological processes associated with memapsin 2 activity rather than diseases or conditions or biological processes associated with both memapsin 2 activity and another peptide hydrolase (such as cathepsin D or memapsin 1).

For example, both memapsin 1 and memapsin 2 cleave amyloid precursor protein (APP) at a β-secretase site to form β-amyloid protein (also referred to herein as Aβ or β-amyloid protein). Thus, both memapsin 1 and memapsin 2 have β-secretase activity (Hussain, I., et al., J. Biol. Chem. 276:23322-23328 (2001)). However, the β-secretase activity of memapsin 1 is significantly less than the β-secretase activity of memapsin 2 (Hussain, I., et al., J. Biol. Chem. 276:23322-23328 (2001)). Memapsin 2 is localized in the brain, and pancreas, and other tissues (Lin, X., et al., Proc. Natl. Acad. Sci. USA 97.1456-1460 (2000)) and memapsin 1 is localized preferentially in placentae (Lin, X., et al., Proc. Natl. Acad. Sci. USA 97:1456-1460 (2000)). Alzheimer's disease is associated with the accumulation of Aβ in the brain as a result of cleaving of APP by β-secretase (also referred to herein as memapsin 2, ASP2 and BACE). Thus, methods employing the compounds which selectively inhibit memapsin 2 activity relative to memapsin 1 activity may be important in the treatment of memapsin 2-related diseases, such as Alzheimer's disease. Selective inhibition of memapsin 2 activity makes the compounds of the invention suitable drug candidates for use in the treatment of Alzheimer's disease.

### VII. METHODS OF TREATING GLAUCOMA

In another aspect of the present invention, the β-secretase inhibitor compounds of the invention can be employed in the treatment of diseases associated with vision loss (e.g., glaucoma). In some embodiments, the invention provides a method of treating glaucoma (e.g., closed-angle glaucoma and open-angle glaucoma) in an individual comprising the step of administering to the individual in need thereof an effective amount of the β-secretase inhibitors of the invention. In an exemplary embodiment, the β-secretase inhibitor compound is part of a pharmaceutical formulation, as described above.

In some aspects, the inhibitor compounds of the invention can be employed in the treatment of diseases or conditions associated with β-secretase activity, which can halt, reverse or diminish the progression of glaucoma (e.g., closed-angle glaucoma and open-angle glaucoma). In some embodiments, the inhibitor compounds of the invention can be used to halt, reverse or diminish the loss of retinal ganglion cells (RGCs). In other embodiments, compounds of the inhibition are employed to improve or decrease intraocular pressure (TOP).

Compounds of the invention may be used to treat glaucoma by one of several known routes of administration, including, but not limited to, orally (e.g., in tablet or capsule form), parenterally (e.g., injected into the anterior chamber, intravenous, intramuscular, or subcutaneous), or locally (e.g., topical eye drops or ointment). Compounds of the invention may also be formulated for sustained release during glaucoma treatment.

Additional embodiments for treating glaucoma with compounds of the invention are described by adapting one or more of the methods in Guo, et. al. Proc. Natl. Acad. Sci., 14, 13444-13449 (2007); Yamamoto, et. al., Neuroscience Letters, 370, 61-64 (2004); and/or Urcola et. al., Exp. Eye Research, 83, 429-437 (2006). The content of these references is hereby incorporated by reference in their entirety.

A. Methods of Administering Beta-Secretase Inhibitors to the CNS

The inhibitor compounds of the present invention may be administered to the CNS through either invasive or non-invasive methods. Non-invasive methods of administration include those methods that do not require the use of a mechanical or physical means to breach the integrity of the blood-brain barrier. Typically, non-invasive methods include the use of immunoliposomes, blood-brain barrier disruption (BBBD), or the olfactory pathway.

Immunoliposomes are liposomes with antibodies or antibody fragments that bind to receptors or transporters expressed on brain capillary endothelial cells attached to the surface of the liposome. An exemplary immunoliposome combines polymer (e.g., PEGylation) technology with that of chimeric peptide technology. For example, the β-secretase inhibitor may be packaged into a unilamellar lipid vesicle containing a PEG²⁰⁰⁰ derivative that contains a reactive groups at one end, for attachment to a complimentary reactive group of an antibody or fragment thereof. Complimentary reactive groups are well known in the art and, include, for example, amine and activated carboxylic acids, thiols and maleimides, and the like (Ambikanandan et al., J. Pharm Pharmaceut Sci 6(2):252-273 (2003); Huwyler et al., Proc. Natl. Acad. Sci. USA, 93:14164-14169 (1996); and Huwyler et al., J Pharmcol Exp Ther. 282:1541-1546 (1997); and U.S. Pat. No. 6,372,250, all of which are herein incorporated by reference for all purposes in their entirety).

Blood-brain barrier disruption is a temporal loss of the integrity of the tight junctions between endothelial cells that comprise the blood brain barrier. Typically, the compound is administered via systemic or intercarotid injection in conjuction with transient blood-brain barrier disruption (BBBD). Exemplary agents useful for inducing BBBD include solvents such as dimethyl sulfoxide (DMSO); ethanol (EtOH); metals (e.g., aluminum); X-irradiation; induction of pathological conditions (e.g., hypertension, hypercapnia, hypoxia, or ischemia); anti-neoplastic agents (e.g., VP-16, cisplatin, hydroxyurea, fluorouracil and etoposide); or concurrent systemic administration of the convulsant drug metrazol and the anti-convulsant drug pentobarbital (Ambikanandan et al., J. Pharm Pharmaceut Sci 6(2):252-273 (2003)); vasoactive leukotrienes (Black et al., J Neurosurg, 81(5):745-751 (1994)); intracarotid infusion of bradykinin, histamine, or the synthetic bradykinin analog RMP-7 (Miller et al., Science 297:1116-1118 (2002), Matsukado, et al., Neurosurgery 39:125-133 (1996), Abbott, et al., Mol Med Today 2:106-113 (1996), Emerich et al., Clin Pharmacokinet 40:105-123 (2001)); hyaluronidase (U.S. Patent Application Publication No. 20030215432, Kreil, et al. Protein Sci., 4(9):1666-1669 (1995)); and intercarotid injection of inert hypertonic solutions such as mannitol, or arabinose (Neuwelt, E. A., et al., in Neuwelt EA (ed), Implications of the Blood Brain Barrier and its Manipulation: Clinical Aspects. Vol. 2, Plenum Press, New York, (1989), Neuwelt, et al., J Nucl Med, 35:1831-1841 (1994), Neuwelt et al., Pediatr Neurosurg 21:16-22 (1994), Kroll et al., Neurosurg, 42:1083-1099 (1998), Rapoport, Cell Mol Neurobiol 20:217-230 (2000), and Doran et al., Neurosurg 36:965-970, (1995)).

Olfactory pathway administration is the intranasal delivery of the compound to the olfactory nerves in the upper third of the nasal passages. After intranasal delivery, the compound is transported back along the sensory olfactory neurons to yield significant concentrations in the cerebral spinal fluid (CSF) and olfactory bulb (Thorne et al., Brain Res, 692(1-2):278-282 (1995); Thorne et al., Clin Pharmacokinet 40:907-946 (2001); Ilium, Drug Discov Today 7:1184-1189 (2002); U.S. Pat. No. 6,180,603; U.S. Pat. No. 6,313,093; and U.S. Patent Application Publication No. 20030215398).

Invasive methods of administration are those methods that involve a physical breach of the blood-brain barrier typically through a mechanical or physical means to introduce the compound into the CSF, or directly into the parenchyma of the brain. Typically, invasive methods of administration may include injection or surgical implantation of the compound.

In injection methods, a needle is used to physically breach the BBB and deliver the compound directly into the CSF. Exemplary injection methods include intraventricular, intrathecal, or intralumbar routes of administration and may also involve infusion of the compound through a reservoir external to the body (Krewson et al., Brain Res 680:196-206 (1995); Harbaugh et al., Neurosurg. 23(6):693-698 (1988); Huang et al., J Neurooncol 45:9-17 (1999); Bobo et al., Proc Natl Acad Sci USA 91:2076-2082 (1994); Neuwalt et al., Neurosurg. 38(4):1129-1145 (1996)).

In surgical implantation methods, the compound is placed directly into the parenchyma of the brain. Exemplary surgical implantation methods may include incorporation of the compound into a polyanhydride wafer placed directly into the interstitium of the brain (Bremet al., Sci Med 3(4):1-11 (1996); Brem et al., J Control Release 74:63-67 (2001)).

### VIII. CRYSTALLIZED COMPLEXES

In another aspect, the present invention provides a crystallized complex containing a memapsin 2 protein and a β-secretase inhibitor of the present invention. Memapsin 2 proteins useful in forming co-crystals with isostere compounds (e.g., memapsin 2 protein fragments, transmembrane proteins, etc.) have been previously discussed in detail (see U.S. Patent Application Publication No. 20040121947, and International Application No. PCT/USO2/34324 (Publication No. WO 03/039454)). These memapsin 2 proteins are equally useful in forming crystallized complexes with β-secretase inhibitors of the present invention.

The crystallized complex may be formed employing techniques described in U.S. Patent Application Publication No. 20040121947, and International Application No. PCT/US02/34324 (Publication No. WO 03/039454). Briefly, a nucleic acid construct encoding the protein is generated, is expressed in a host cell, such as a mammalian host cell (e.g., Hela cell, 293 cell) or a bacterial host cell (e.g., E. coli), is purified and is crystallized with a compound or compounds of the invention. The diffraction resolution limit of the crystallized protein can be determined, for example, by x-ray diffraction or neutron diffraction techniques.

In an exemplary embodiment, the crystallized protein may have an x-ray diffraction resolution limit not greater than about 4.0 Δ. The crystallized protein may also have an x-ray diffraction resolution limit not greater than about 4.0 Δ, about 3.5 Δ, about 3.0 Δ, about 2.5 Δ, about 2.0 Δ, about 1.5 Δ, about 1.0 Δ, or about 0.5 Δ. In some embodiments, the crystallized protein may also have an x-ray diffraction resolution limit not greater than about 2 Δ. The diffraction resolution limit of the crystallized protein can be determined employing standard x-ray diffraction techniques.

In an other exemplary embodiment, the β-secretase inhibitor of the crystallized complex is in association with said protein at an S₃′ binding pocket, an S₄′ binding pocket and/or an S₄ binding pocket. S₃′, S₄′, and S₄ binding pockets are discussed in detail in U.S. Patent Application Publication No. 20040121947, and International Application No. PCT/USO2/34324 (Publication No. WO 03/039454).

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the β-secretase inhibitors of the present invention are equally applicable to the methods of treating disease states and/or the pharmaceutical compositions described herein. All publications, patents, and patent applications cited herein are hereby incorporated by reference in their entirety for all purposes.

### IX. EXAMPLES

### Example 1

### Preparation of Selected Beta-Secretase Inhibitors and Precursor Compounds

The described synthesis of Beta-Secretase inhibitors and precursor compounds is related to WO 2006/110668, filed on Apr. 10, 2006 and entitled “Compounds Which Inhibit Beta-Secretase Activity and Methods of Use Thereof,” the content of which is incorporated herein by reference in its entirety, and particularly with respect to the synthetic methods described therein, e.g., paragraphs 150-153 and paragraphs 215-285; and U.S. Provisional Patent Application No. 60/952,198, filed on Jul. 26, 2007 and entitled “Compounds Which Inhibit Beta-Secretase Activity and Methods of Use Thereof,” the content of which is incorporated herein by reference in its entirety, and particularly with respect to the synthetic methods described therein, e.g., paragraphs 83-86 and paragraphs 161-354.

The precursor compounds synthesized below are useful in the methods of making compounds of the present invention provided herein. Using the guidance provided, (for example, in the Exemplary Syntheses of Scheme 1) one skilled in the art will immediately recognize that the exemplified synthesis of the below precursor compounds may be modified using well known techniques and the teaching provided herein to arrive at a wide variety of inhibitor compounds. Certain starting materials described, and some precursor compounds not described, may be commercially available and purchased from, for example, Sigma-Aldrich, Alfa Aesar, or Ryan Scientific.

NMR spectra were collected on a Varian Mercury model VX-300 NMR spectrometer. NMR solvents were purchased from Cambrige Isotope Laboratories.

Solvents used in the synthesis of inhibitor compounds were purchased from Aldrich, VWR, and EMD. Solvents were ACS Reagent Grade or higher, and used without further purification.

### Example 1.1

### Synthesis of Amine Building Blocks

### Example 1.1.1

### (4-methylthiazol-2-yl)methanamine

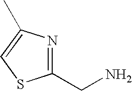

Methylthiazole (1.0 g, 10.1 mmol) in THF at −78° C. was treated with n-BuLi (1.6 M, 7.56 mL) for 30 min, DMF (1.4 mL, 18.2 mmol) was added dropwise. The resulting reaction mixture was warmed to r.t. After the starting material disappeared (by TLC), the reaction mixture was recooled to 0° C. and LAH (0.69 g, 18.5 mmol) was added. The mixture was warmed to r.t. and stirred for 1 h, the reaction was quenched with aqueous NH₄Cl, diluted with EtOAc. The organic solution was separated, extracted twice with EtOAc, dried with Na₂SO₄, and concentrated. The residue was purified with flash chromatography to give the corresponding alcohol as a light yellow oil. ¹H-NMR: (300 MHz, CDCl₃), d: 6.89 (s, 1H); 4.95 (s, 2H); 2.48 (s, 3H).

Methylthiazole methanol (0.57 g, 4.4 mmol) was treated with mesyl chloride (0.42 mL, 5.4 mmol) and triethyl ethylamine at 0° C. in dichloromethane. The resulting mixture was stirred for 20 minutes followed by quenching with aqueous NH₄Cl. Evaporation of the solvent from the organic layer and flash chromatography of the residue afforded the corresponding mesylate as an oil. The mesylate (0.25 g, 1.2 mmol) was then dissolved in DMF and sodium azide (0.62 g, 9.6 mmol) was added. The mixture was heated to reflux for 2 hours followed by cooling and washing with aqueous NH₄Cl. Evaporation of the solvent from the organic layer resulted in the corresponding azide. The azide (0.14 g, 0.91 mmol) was dissolved in ethyl acetate, Pd(OH)₂ (0.07 g) was added, and the suspension was stirred under a hydrogen atmosphere for 5 hours. The suspension was filtered through Celite. Evaporation of the solvent and flash chromatography of the residue afforded methylthiazole methylamine as a yellow oil. ¹H-NMR: (300 MHz, CDCl₃), d: 6.74 (m, 1H); 4.09 (m, 2H); 2.37 (s, 3H).

### Example 1.1.2

### 1-(pyridin-3-yl)ethanamine

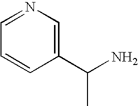

To a solution of 3-acetylpridine (82.6 mmol) in methanol (200 mL) was added ammonium acetate (1.03 mol) in one portion at room temperature. After the mixture was stirred for 20 min, sodium cyanoborohydride (57.8 mmol) was added. After being stirring for one day, 6 M hydrochloric acid was added. The resulting solution was washed with diethyl ether, and then the aqueous phase was basified to pH=10 with potassium hydroxide. The liberated amine was extracted with chloroform, and the combined organic extracts were dried over anhydrous sodium sulfate. After removal of the solvent under reduced pressure, the crude amine was obtained as a colorless oil, which was further purified by distillation under reduced pressure. ¹H NMR (300 MHz, CDCl₃), d: 8.552 (d, 1H), 8.453 (dd, 1H), 7.678 (m, 1H), 7.206-7.261 (m, 1H), 4.148 (q, 1H), 1.378 (d, 3H).

### Example 1.1.3

### 1-(4-methylthiazol-2-yl)ethylcarbamate

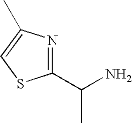

A mixture of Boc-alanine-thioamide (1.39 g, 6.81 mmol), chloroacetone (0.65 mL, 8.18 mmol) and calcium carbonate (1.0 g, 10.22 mmol) were refluxed in ethanol (25 mL) for 4 h. The reaction was cooled to room temperature and quenched with 20 mL of saturated aq. NaHCO₃ solution. Ethanol was evaporated under reduced pressure and extracted with ethyl acetate (2×30 mL). The combined organic layers was dried over Na₂SO₄ and concentrated. The residue was chromatographed on silica gel (20% ethyl acetate/80% hexane) to yield 48% of the Boc-protected product. The desired 1-(4-methylthiazol-2-yl)ethylcarbamate was then generated by treatment with HCl (in methanol or dioxane) or trifluoroacetic acid in dichloromethane.

### Example 1.1.4

### (4-isopropylpyridin-2-yl)methanamine

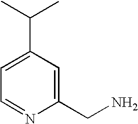

To a stirring mixture of 5.1 g (14.3 mmol) of Ph₃PCH₃Br in 25 mL of THF at 0° C. was added 11.0 mL of n-BuLi (1.6M in hexanes) dropwise over a period of 20 min. After 1 h, 1.5 g (12.8 mmol) of 1-(pyridin-4-yl)ethanone was added in 20 mL of THF. The mixture was stirred at 0° C. for 1 h and then at r.t for 50 min. The mixture was filtered through a Buchner funnel. Saturated NH₄Cl and H₂O were added, and the layers were separated. The organic layer was washed with brine, dried over Na₂SO₄, filtered, and concentrated. Purification by flash silica gel chromatography (54% EtOAc/hexanes) provided the isopropenyl pyridine as a pale yellow liquid.

A mixture of 4-(prop-1-en-2-yl)pyridine and 342 mg of 20% Pd(OH)₂ in 15 mL of EtOAc and 10 mL of MeOH was stirred under H₂ balloon at r.t. After 24 h, 305 mg of 20% Pd(OH)₂ was added, and after 6 h the mixture was filtered through Celite, filtered, and concentrated. The crude product was dissolved in 15 mL of MeOH and 512 mg of 20% Pd(OH)₂ was added. The mixture was stirred under H₂ balloon for 11.5 h at r.t., filtered through Celite, and concentrated to give the 4-isopropylpyridine which was used without further purification.

A solution of 4-isopropylpyridine and 1.5 mL of 30% H₂O₂ in 7 mL of AcOH was heated at 135° C. A total of 15.4 mL of H₂O₂ was added in 4 portions, and the solution was refluxed for 2 h. Chloroform and water were added, the layers were separated, and the aqueous layer was extracted 3 times with CHCl₃. The combined extracts were dried over Na₂SO₄, filtered, and concentrated. Purification by flash silica gel chromatography (5% MeOH/CHCl₃) provided 139 mg of the 4-isopropylpyridine N-oxide as an orange oil.

To a stirring solution of 139 mg (1.01 mmol) of 4-isopropylpyridine N-oxide in 10 mL of CH₂Cl₂ at r.t. was added 160 μL (1.20 mmol) of TMSCN. After 5 min., 100 μL (1.09 mmol) of dimethylcarbamyl chloride was added, and the solution was stirred at r.t. for 16 h. The solution was diluted with chloroform and washed with 20 mL of 10% aqueous K₂CO₃. The layers were separated, and the aqueous layer was extracted 3 times with CHCl₃. The combined extracts were dried over Na₂SO₄, filtered, and concentrated. Purification by flash silica gel chromatography (30% EtOAc/hexanes) provided 4-isopropylpicolinonitrile with some impurity as a liquid.

To a stirring solution of 149 mg (1.01 mmol) of 4-isopropylpicolinonitrile in 7 mL of THF at 0° C. was added 150 mg (3.95 mmol) of LiAlH₄. After about 15 min., 250 mg of LiAlH₄ was added, and after about 15 min. the ice bath was removed and stirring was continued with warming to r.t. After 40 min., 400 μL of H₂O, 400 μL of 15% NaOH (aq), and 1.2 mL of brine were added in succession. The mixture was stirred for 85 min., filtered through Celite, and conc. to give 143 mg of (4-isopropylpyridin-2-yl)methanamine which was used without further purification.

### Example 1.1.5

### (6-isopropylpyridin-2-yl)methanamine

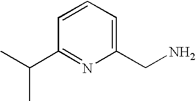

(6-isopropylpyridin-2-yl)methanamine was synthesized from the ketone following the general procedure as described for 4-isopropyl-2-pyridylmethylamine.

### Example 1.1.6

### (2-isopropylpyridin-4-yl)methanamine

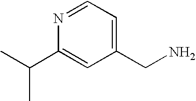

To a solution of 3.1 g (29.8 mmol) of 4-cyanopyridine and 7.7 g (87.4 mmol) of pyruvic acid in 150 mL of CH₂Cl₂ was added a solution of 150 mL of H₂O with 3.0 mL H₂SO₄ and 9.9 g (43.4 mmol) of (NH₄)₂S₂O₈. To this mixture was added 440 mg of AgNO₃. Let mixture stir with the light off for 2 h at 40° C. Solid NaOH was added to a pH=8, and the aqueous layer was extracted with CHCl₃. The extract was dried over Na₂SO₄, filtered, and concentrated. Flash silica gel chromatography (10% EtOAc/hexanes) provided 1.81 g colorless solid of the corresponding ketone (2-acetylisonicotinonitrile) with some impurity.

The isopropenyl pyridine (2-(prop-1-en-2-yl)isonicotinonitrile) was synthesized from 2-acetylisonicotinonitrile following the general procedure as described for 4-isopropyl-2-pyridylmethylamine.

A mixture of 154 mg (1.07 mmol) of 2-(prop-1-en-2-yl)isonicotinonitrile and 19.2 mg of 10% Pd/C with 1.7 mL of 1.25 N HCl in 10 mL of MeOH was stirred at r.t. under H₂ balloon for 14 h. The mixture was filtered through Celite and concentrated. Saturated NaHCO₃ was added, and the mixture concentrated with methanol, filtered through a sintered funnel, and reconcentrated to give 95.1 mg of crude (2-isopropylpyridin-4-yl)methanamine which was used without further purification.

### Example 1.1.7

### methyl 6-(aminomethyl)-2-methylnicotinate

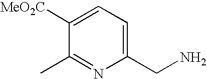

methyl 2-methylnicotinate was synthesized from 2-methylnicotinic acid following the general procedure as described for dimethylpyridine-3,5-dicarboxylate.

methyl 6-cyano-2-methylnicotinate was synthesized from methyl 2-methylnicotinate following the general procedure as described for 4-isopropyl-2-pyridylmethylamine.

methyl 6-(aminomethyl)-2-methylnicotinate was synthesized from methyl 6-cyano-2-methylnicotinate following the general procedure as described for 2-isopropyl-4-pyridylmethylamine.

### Example 1.1.8

### (2-fluoro-5-isopropylpyridin-3-yl)methanamine

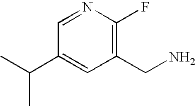

To a stirring mixture of 3.0 g (21.3 mmol) of boronic acid in 60 mL of THF and 30 mL of 2.0 M Na₂CO₃ (degassed) was added 1.31 g (1.14 mmol) of Pd(PPh₃)₄ followed by 2.8 mL (32.1 mmol) of 2-bromopropene. The mixture was stirred at 40° C. under Ar. After 165 min., 2.0 mL of 2-bromopropene was added and heating was continued for 140 min. Diethyl ether was added, and the layers were separated. The organic layer was dried over Na₂SO₄, filtered, and concentrated. 2-fluoro-5-(prop-1-en-2-yl)pyridine was used without further purification.

2-fluoro-5-isopropylpyridine was synthesized from 2-fluoro-5-(prop-1-en-2-yl)pyridine following the general procedure as described for 4-isopropylpyridine.

To a stirring solution of 9.2 mL of LDA (lithium diisopropylamide) (2.0M solution in THF/heptane/ethylbenzene) in 20 mL of THF at −78° C. was added 2-fluoro-5-isopropylpyridine in 40 mL of THF dropwise over a period of 20 min. After 30 min. 4.39 g (17.3 mmol) of iodine in 25 mL of THF was added. After 2 h at −78° C. with the light off, 20 mL of water was added, and the cold bath was removed. Water (80 mL) was added and 9 g of sodium thiosulfate was added in 3 portions. Diethyl ether was added, and the layers were separated. The organic layer was washed with brine, dried over Na₂SO₄, filtered, and concentrated. Purification by flash silica gel chromatography (5% Et₂O/hexanes) provided 1.83 g of 2-fluoro-3-iodo-5-isopropylpyridine with some impurity.

To a mixture of 2-fluoro-3-iodo-5-isopropylpyridine and 485 mg (4.13 mmol) of Zn(CN)₂ in 15 mL of DMF (degassed) was added 360 mg (0.312 mmol) of Pd(PPh₃)₄. The mixture was heated at 80° C. under Ar for 19 h. The mixture was diluted with EtOAc, and the organic layer was washed with 10% NH₄OH (2×10 mL), H₂O (3×), and brine. The organic layer was dried over Na₂SO₄, filtered, and concentrated. Purification by flash silica gel chromatography (5% Et₂O/hexanes) provided 384 mg of pure 2-fluoro-5-isopropylnicotinonitrile as a yellow liquid.

To a stirring solution of 238 mg (1.44 mmol) of 2-fluoro-5-isopropylnicotinonitrile in 7 mL of THF at 0° C. was added 102 mg (2.7 mmol) of LiAlH₄. After 20 min., 104 mg of LiAlH₄ was added and after 10 min, the mixture was allowed to warm to r.t. and after another 20 min. the mixture was heated to 50-60° C. The reaction did not go to completion after 30 min., and the reaction was then quenched with 200 μL of H₂O, 200 μL of 15% NaOH aqueous, and 600 μL of brine were added. EtOAc was added and stirring was continued at r.t. The mixture was filtered through Celite and concentrated.

To a stirring mixture of crude product and 382 mg (1.61 mmol) of CoCl₂.6H₂O in 7 mL of EtOH at 50° C. was added 259 mg (6.85 mmol) of NaBH₄ in 2 portions. After 10 min., 56.2 mg of NaBH₄ was added. After 2 h at 50° C., 5N HCl was added to a pH=1-2, and the mixture was stirred until the bubbling ceased. The mixture was concentrated, and NH₄OH was added to a pH=8. The aqueous layer was extracted with the extract of (40 mL CHCl₃: 5 mL H₂O: 5 mL MeOH) 3 times. The combined extracts were dried over Na₂SO₄, filtered, and concentrated to give 165 mg of crude (2-fluoro-5-isopropylpyridin-3-yl)methanamine which was used without further purification.

### Example 1.1.9

### (5-isopentylpyridin-3-yl)methanamine

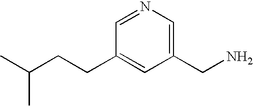

To a stirring mixture of 4.26 g (10.7 mmol) of Ph₃PCH₂CH(CH₃)₂Br in 20 mL of THF 0° C. was added 12.0 mL of n-BuLi (1.6 M in hexanes). The mixture was stirred at 0° C. for 1 h, and then 521 mg (2.82 mmol) of 5-bromonicotinaldehyde in 20 mL of THF was added. After 5-10 min. the bath was removed and stirring was continued with warming to r.t. After 1 h, the reaction was quenched with water and diluted with EtOAc. The organic layer was washed with brine, dried over Na₂SO₄, filtered, and concentrated. Purification by flash silica gel chromatography 15% EtOAc/hexanes provided (E)-3-bromo-5-(3-methylbut-1-enyl)pyridine and impurity.

(E)-5-(3-methylbut-1-enyl)nicotinonitrile was synthesized from (E)-3-bromo-5-(3-methylbut-1-enyl)pyridine following the general procedure as described for the 2-fluoro-5-isopropylnicotinonitrile.

To a stirring mixture of 536 mg of cyanide and 789 mg (3.32 mmol) of CoCl₂.6H₂O in 10 mL of EtOH at 50° C. was added 749 mg (19.8 mmol) of NaBH₄ in 3 portions. After 2 h at 50° C., 5N HCl was added to a pH=1-2 and stirring was continued until the bubbling ceased. The reaction mixture was concentrated and NH₄OH was added to a pH=9. The aqueous layer was extracted with the extract of (40 mL CHCl₃: 5 mL MeOH: 5 mL H₂O) (2×). The combined extracts were dried over Na₂SO₄, filtered, and concentrated. Crude (5-isopentylpyridin-3-yl)methanamine was used in the next reaction without purification.

### Example 1.1.10

### benzyl 3-(aminomethyl)-5-isopropylphenylcarbamate

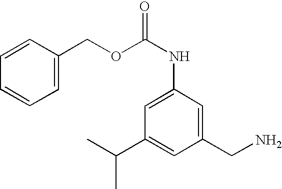

To a stirring mixture of 1.2 g (4.96 mmol) of 2-amino-3-bromo-5-nitrobenzonitrile and 2.8 mL of H₂SO₄ in 28 mL of EtOH at 90° C. was added 2.5 g (36.2 mmol) of NaNO₂ in several portions. After 13.5 h, EtOAc and H₂O were added. The organic layer was washed with H₂O and brine, dried over Na₂SO₄, filtered, and concentrated. Purification by flash silica gel chromatography (5% EtOAc/hexanes) provided 927 mg of 3-bromo-5-nitrobenzonitrile in 82% yield as a yellow solid.

To a stirring solution of 1.8 g of 3-bromo-5-nitrobenzonitrile in 8 mL of EtOH and 8 mL of THF was added 8.8 g (38.6 mmol) of SnCl₂.2H₂0 in several portions. The mixture was allowed to stir at r.t. for 10.5 h and then concentrated. A solution of 2N NaOH (60 mL) was added and stirring continued for 2 h. EtOAc was added, and the layers were separated. The organic layer was washed with H₂O and brine. The aqueous layer was reextracted with EtOAc and washed with brine. The combined extracts were dried over Na₂SO₄, filtered, and concentrated. Purification by flash silica gel chromatography (35% EtOAc/hexanes) provided 965 mg of 3-amino-5-bromobenzonitrile.

To a stirring of 965 mg (4.90 mmol) of 3-amino-5-bromobenzonitrile in 15 mL of CH₂Cl₂ was added 1.27 g (5.09 mmol) of N-(benzyloxycarbonyloxy)succinimide (followed by 5 mL of CH₂Cl₂), and 1.5 mL (10.8 mmol) of Et₃N. After 27 h, the solution was concentrated and 10 mL of 10% citric acid and EtOAc were added. The layers were separated, and the organic layer was washed with 10 mL of 10% citric acid, 20 mL of H₂O, and 10 mL of brine, dried over Na₂SO₄, filtered, and concentrated. Purification by flash silica gel chromatography (25% EtOAc/hexanes) provided 773 mg of Cbz protected product (benzyl 3-bromo-5-cyanophenylcarbamate) with some impurity.

Benzyl 3-(aminomethyl)-5-bromophenylcarbamate was synthesized from benzyl 3-bromo-5-cyanophenylcarbamate following the general procedure as described for the 3-cyano-5-isopentylpyridine.

A solution of crude benzyl 3-(aminomethyl)-5-bromophenylcarbamate, 1.6 mL (11.5 mmol) of Et₃N, and 0.6 mL (2.61 mmol) of Boc₂O in 15 mL of MeOH was stirred at r.t. 17 h. The solution was concentrated, water and EtOAc were added, and the layers were separated. The organic layer was washed with H₂O and brine, dried over Na₂SO₄, filtered, and concentrated. Purification by flash silica gel chromatography (15% EtOAc/hexanes) provided the Boc-protected benzyl 3-(aminomethyl)-5-bromophenylcarbamate with some impurity.

A solution of 400 mg of Boc-protected benzyl 3-(aminomethyl)-5-bromophenylcarbamate, 153 mg (1.02 mmol) of potassium isopropenyltrifluoroborate, and 0.4 mL (2.87 mmol) of Et₃N in 40 mL of isopropanol and 20 mL of H₂O (degassed) was added 42.1 mg (0.0516 mmol) of PdCl₂(dppf) CH₂Cl₂. After heating the solution for 2 h, 100 mL of H₂O and EtOAc were added. The organic layer was washed with brine, dried over Na₂SO₄, filtered, and concentrated. Purification by flash silica gel chromatography (20% EtOAc/hexanes) provided 126 mg of the Boc-protected benzyl 3-(aminomethyl)-5-(prop-1-en-2-yl)phenylcarbamate as a yellow liquid in 66% yield.

The desired benzyl 3-(aminomethyl)-5-isopropylphenylcarbamate was synthesized from the Boc-protected benzyl 3-(aminomethyl)-5-(prop-1-en-2-yl)phenylcarbamate following the general procedure as described above for the 3-cyano-5-isopentylpyridine.

### Example 1.1.11

### (6-methyl-5-(methylthiomethyl)pyridin-2-yl)methanamine

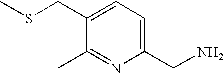

To a stirring solution of 1.05 g of methyl 6-cyano-2-methylnicotinate in 45 mL of MeOH at 0° C. was added 605 mg of NaBH₄. During a period of 45 min, 2 portions (a total of 1.26 g) of NaBH₄ was added. After 1 h, 1N HCl was added to a pH=7, and the aqueous layer was extracted with CHCl₃. The organic layer was dried over Na₂SO₄, filtered, and concentrated. The crude alcohol (5-(hydroxymethyl)-6-methylpicolinonitrile) was used for the next reaction without further purification.

To a stirring solution of 5-(hydroxymethyl)-6-methylpicolinonitrile in 15 mL of CH₂Cl₂ was added 0.9 mL (6.46 mmol) of Et₃N and 46.0 mg (0.377 mmol) of DMAP at r.t. The solution was cooled to 0° C. and 0.7 mL of MsCl was added. The solution was cooled to 0° C. for 45 min. and 20 mL of H₂O, CH₂Cl₂ and CHCl₃ were added. The layers were separated and the organic layer was washed with brine, dried over Na₂SO₄, filtered, and concentrated. Purification by flash silica gel chromatography ((0.6-1.0) % MeOH/CHCl₃) resulted in 919 mg of (6-cyano-2-methylpyridin-3-yl)methyl methanesulfonate as a yellow liquid in 68% yield.

A mixture of 919 mg (4.05 mmol) of (6-cyano-2-methylpyridin-3-yl)methyl methanesulfonate and 360 mg (5.14 mmol) of NaSMe in 15 mL of EtOH was heated at 95° C. for 4 h. Saturated NaHCO₃ solution and EtOAc were added, and the layers were separated. The organic layer was washed with brine, dried over Na₂SO₄, filtered, and concentrated. Purification by flash silica gel chromatography (50% Et₂O/hexanes) provided 46.6 mg of 6-methyl-5-(methylthiomethyl)picolinonitrile as a pale yellow solid in 6.4% yield.

(6-methyl-5-(methylthiomethyl)pyridin-2-yl)methanamine was synthesized from 6-methyl-5-(methylthiomethyl)picolinonitrile following the general procedure as described for the 3-cyano-5-isopentylpyridine.

### Example 1.1.12

### 3-(aminomethyl)-5-isopropyl-N,N-dimethylaniline

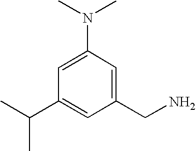

To a stirring suspension of 210 mg (10.6 mmol) of 3-amino-5-bromobenzonitrile in 11 mL of CH₃CN and 11 mL of 37% formaldehyde in H₂O was added 222 mg (3.53 mmol) of NaCNBH₃ followed by about 7 drops of acetic acid to a pH=7. After 1 h 40 min., an additional amount of acetic acid (10 drops) was added. After 15 h, 40 mL of EtOAc and 40 mL of sat. NaHCO₃ was added, and the layers were separated. The organic layer was washed with 25 mL of sat. NaHCO₃ and 25 mL of brine, dried over Na₂SO₄, filtered, and concentrated. Purification by flash silica gel chromatography (10% EtOAc/hexanes) provided 67.4 mg of 3-bromo-5-(dimethylamino)benzonitrile as a yellow solid in 28% yield.

3-(dimethylamino)-5-(prop-1-en-2-yl)benzonitrile was synthesized from 3-bromo-5-(dimethylamino)benzonitrile following the general procedure as described for the Cbz protected Boc aminomethyl bromide.

3-(aminomethyl)-5-isopropyl-N,N-dimethylaniline was synthesized from 3-(dimethylamino)-5-(prop-1-en-2-yl)benzonitrile following the general procedure as described for the 3-cyano-5-isopentylpyridine.

### Example 1.1.13

### (5-isopropyl-2,6-dimethylpyridin-3-yl)methanamine

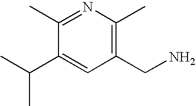

5-(methoxycarbonyl)-2,6-dimethylnicotinic acid was synthesized from 2,6-dimethylpyridine-3,5-dicarboxylic acid following the general procedure as described for the pyridine-3,5-dicarboxylic acid.

A solution of 686 mg of 5-(methoxycarbonyl)-2,6-dimethylnicotinic acid and 8.0 mL of BH₃.THF (1.0 M in THF) in 10 mL of THF was heated to 75° C. for 2.5 h. An aqueous solution of 3 mL of AcOH:H₂O (1:1) was added, and the solution was stirred until the bubbling ceased. Saturated NaHCO₃ was added to a pH=7, and the solution was stirred overnight. EtOAc and H₂O were added, and the layers were separated. The aqueous layer was extracted with 30 mL of EtOAc, and the combined extracts were washed with brine, dried over Na₂SO₄, filtered, and concentrated. Purification by flash silica gel chromatography (3% MeOH/CHCl₃) provided 452 mg of methyl 5-(hydroxymethyl)-2,6-dimethylnicotinate as a pale yellow solid in 71% yield.

To a stirring solution of 5.0 mL of MeMgCl (3.0 M in THF) in 20 mL of THF at 0° C. was added 452 mg (2.31 mmol) of methyl 5-(hydroxymethyl)-2,6-dimethylnicotinate in 40 mL of THF dropwise over a period of about 25 min. The solution was stirred at 0° C. for 45 min. and then at rt. for about 2.5 h. Another portion of MeMgCl (4 mL) was added and stirring was continued for about 2 h 20 min. Saturated NH₄Cl solution (60 mL), 15 mL of H₂O, and EtOAc were added, and the layers were separated. The aqueous layer was extracted with EtOAc (2×), and the combined extracts were washed with brine (60 mL), dried over Na₂SO₄, filtered, and concentrated. 2-(5-(hydroxymethyl)-2,6-dimethylpyridin-3-yl)propan-2-ol (313 mg) was used for the next reaction without further purification.

To a stirring solution of 2-(5-(hydroxymethyl)-2,6-dimethylpyridin-3-yl)propan-2-ol in 10 mL of CH₂Cl₂ was added 1.5 mL of SO₂Cl₂. After 11 h at r.t., the solution was concentrated and CH₂Cl₂ and 10 mL of sat. NaHCO₃ solution were added, and the layers were separated. The organic layer was washed with brine, dried over Na₂SO₄, filtered, and concentrated. Purification by flash silica gel chromatography (20% Et₂O/hexanes) provided 96.5 mg of the desired 3-(chloromethyl)-2,6-dimethyl-5-(prop-1-en-2-yl)pyridine as a mixture.

A mixture of 96.5 mg of the 3-(chloromethyl)-2,6-dimethyl-5-(prop-1-en-2-yl)pyridine and 86.8 mg (1.34 mmol) of NaN₃ in 3 mL of DMF was heated at 70° C. for 4 h. Water and EtOAc were added, and the layers were separated. The organic layer was washed with H₂O (2×) and then with brine. It was dried over Na₂SO₄, filtered, and concentrated to give the crude azide (3-(azidomethyl)-2,6-dimethyl-5-(prop-1-en-2-yl)pyridine) which was used without further purification.

A mixture of crude 3-(azidomethyl)-2,6-dimethyl-5-(prop-1-en-2-yl)pyridine and 10.2 mg of 10% Pd/C in 6 mL of MeOH was stirred at r.t. under H₂ balloon. After 3 h the mixture was filtered through Celite and concentrated to provide (2,6-dimethyl-5-(prop-1-en-2-yl)pyridin-3-yl)methanamine.

To a solution of crude (2,6-dimethyl-5-(prop-1-en-2-yl)pyridin-3-yl)methanamine and 120 mg (0.505 mmol) of CoCl₂.6H₂O in 5 mL of EtOH at 50° C. was added 115 mg of NaBH₄ in 2 portions. After 2 h, 5N HCl was added to a pH=1 and stirring was continued until the bubbling ceased. The mixture was concentrated, and NH₄OH was added to pH=9. Some water was added, and the aqueous layer was extracted with the extract of (40 mL CHCl₃: 5 mL MeOH: 5 mL H₂O). The combined extracts were dried over Na₂SO₄, filtered, and concentrated to give the crude (5-isopropyl-2,6-dimethylpyridin-3-yl)methanamine which was used without further purification.

### Example 1.1.14

### (6-methyl-5-(methylsulfonylmethyl)pyridin-2-yl)methanamine

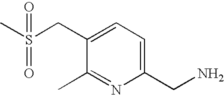

To a stirring solution of 1.5 g of methyl 2-methylnicotinate in 20 mL of THF at 0° C. was added 844 mg of LiAlH₄. After 20 min., 0.84 mL of H₂O, 0.84 mL of 15% aqueous NaOH, and 2.5 mL of brine. EtOAc was added, the ice bath removed, and stirring was continued for 1 h. The mixture was filtered through Celite and concentrated to give 889 mg of crude (2-methylpyridin-3-yl)methanol which was used without further purification.

To a stirring solution of crude (2-methylpyridin-3-yl)methanol in 20 mL of CH₂Cl₂ was added 2.2 mL of SO₂Cl₂. After 17 h the solution was concentrated and CH₂Cl₂ and saturated NaHCO₃ were added. The layers were separated, and the organic layer was washed with brine, dried over Na₂SO₄, filtered, and concentrated. Purification by flash silica gel chromatography (1% MeOH/CHCl₃) provided 1.18 g of 3-(chloromethyl)-2-methylpyridine as yellow-orange oil.

A mixture of 3-(chloromethyl)-2-methylpyridine and 619 mg (8.83 mmol) of NaSMe in 10 mL of EtOH was heated at 95° C. for 6 h. Saturated NaHCO₃ (10 mL) and EtOAc were added, and the layers were separated. The organic layer was washed with brine, dried over Na₂SO₄, filtered, and concentrated. Purification by flash silica gel chromatography (1% MeOH/CHCl₃) provided 801 mg of 2-methyl-3-(methylthiomethyl)pyridine as a brown liquid in 72% yield.

A solution of 801 mg (5.26 mmol) of 2-methyl-3-(methylthiomethyl)pyridine and 5 mL of 30% H₂O₂ in 5 mL of AcOH was heated at 120° C. After 2 h, 3 mL of 30% H₂O₂ was added, and after about 3.5 h, the solution was concentrated. The solution was diluted with CHCl₃ and H₂O, the layers were separated, and the organic layer was extracted with the extract of (40 mL CHCl₃: 5 mL MeOH: 5 mL H₂O) The combined extracts were dried over Na₂SO₄, filtered, and concentrated. Purification by flash silica gel chromatography (10 mL MeOH/100 mL CHCl₃) provided 2-methyl-3-(methylsulfonylmethyl)pyridine N-oxide as a pale yellow solid in about 60% yield.

6-methyl-5-(methylsulfonylmethyl)picolinonitrile was synthesized from 2-methyl-3-(methylsulfonylmethyl)pyridine N-oxide following the general procedure as described for the 4-isopropyl-2-pyridylmethylamine.

(6-methyl-5-(methylsulfonylmethyl)pyridin-2-yl)methanamine was synthesized from 6-methyl-5-(methylsulfonylmethyl)picolinonitrile following the general procedure as described above for the 3-cyano-5-isopentylpyridine.

### Example 1.1.15

### (2,6-diisopropylpyridin-4-yl)methanamine

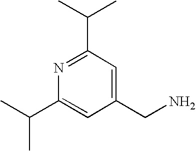

A solution of 1.14 g (6.2 mmol) of chelidamic acid hydrate (4-oxo-1,4-dihydropyridine-2,6-dicarboxylic acid) and about 10 g of PBr₅ was heated at 90° C. under a CaCl₂ drying tube for 3.5 h. Chloroform (125 mL) was added, the mixture was filtered, and to the solution in a 500 mL round bottom flask was added 30 mL of MeOH dropwise over a period of 1 h. After 1 h, 120 mL of saturated NaHCO₃ was added, and the layers were separated. The organic layer was washed with brine, dried over Na₂SO₄, filtered, and concentrated. Crude dimethyl 4-bromopyridine-2,6-dicarboxylate (1.72 g) was used without further purification.

2,2′-(4-bromopyridine-2,6-diyl)dipropan-2-ol was synthesized from dimethyl 4-bromopyridine-2,6-dicarboxylate following the general procedure as described for 2,6-dimethyl 3,5-pyridyl derivative.

A solution of 1.45 g (5.28 mmol) of 2,2′-(4-bromopyridine-2,6-diyl)dipropan-2-ol and 585 mg (6.53 mmol) of CuCN in 20 mL of DMF was heated at 150° C. After about 20.5 h, 40 mL of H₂O and EtOAc was added, and the mixture was filtered through a Buchner funnel. The layers were separated, and the organic layer was washed with water (3×30 mL) and brine (30 mL). The organic layer was dried over Na₂SO₄, filtered, and concentrated. Purification by flash silica gel chromatography (1% MeOH/CHCl₃) provided 373 mg of 2,6-bis(2-hydroxypropan-2-yl)isonicotinonitrile as a pale orange solid in 32% yield.

2,6-di(prop-1-en-2-yl)isonicotinonitrile was synthesized from 2,6-bis(2-hydroxypropan-2-yl)isonicotinonitrile following the general procedure as described for 2,6-dimethyl 3,5-pyridyl.

(2,6-diisopropylpyridin-4-yl)methanamine was synthesized from 2,6-di(prop-1-en-2-yl)isonicotinonitrile following the general procedure as described for 3-cyano-5-isopentylpyridine.

### Example 1.1.16

### (3-(benzyloxy)-5-isopropylphenyl)methanamine

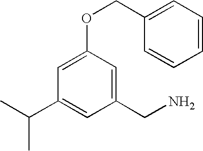

A mixture of 1.98 g (9.42 mmol) of dimethyl 5-hydroxyisophthalate, 3.0 g (21.7 mmol) of K₂CO₃, and 1.8 mL (15.1 mmol) of BnBr in 30 mL of DMF was heated at 60° C. After 17.5 h, the mixture was filtered through cotton, the solution was diluted with CHCl₃ and the organic layer washed with water, and brine. The organic layer was dried over Na₂SO₄, filtered, and concentrated. Purification by flash silica gel chromatography (15% EtOAc/hexanes) resulted in 2.64 g of dimethyl 5-(benzyloxy)isophthalate as a colorless solid in 93% yield.

1-(azidomethyl)-3-(benzyloxy)-5-(prop-1-en-2-yl)benzene was synthesized from dimethyl 5-(benzyloxy)isophthalate following the general procedure as described for 2,6-dimethyl 3,5-pyridyl.

A solution of 392 mg (1.35 mmol) of 1-(azidomethyl)-3-(benzyloxy)-5-(prop-1-en-2-yl)benzene and 525 mg (2.00 mmol) of PPh₃ in 5 mL of THF and 0.5 mL of H₂O was stirred at r.t. for 19 h. The solution was concentrated, diluted with EtOAc, dried over Na₂SO₄, filtered, and concentrated. Crude (3-(benzyloxy)-5-(prop-1-en-2-yl)phenyl)methanamine was used for the next reaction without purification.

The Boc protected (3-(benzyloxy)-5-(prop-1-en-2-yl)phenyl)methanamine was synthesized from (3-(benzyloxy)-5-(prop-1-en-2-yl)phenyl)methanamine following the general procedure as described for the Cbz protected Boc aminomethyl bromide.

tert-butyl 3-(benzyloxy)-5-isopropylbenzylcarbamate was synthesized from the Boc protected (3-(benzyloxy)-5-(prop-1-en-2-yl)phenyl)methanamine following the general procedure as described for 3-cyano-5-isopentylpyridine.

A solution of tert-butyl 3-(benzyloxy)-5-isopropylbenzylcarbamate and 15.0 mL of 1.25 M HCl in MeOH was stirred at r.t. for 3.5 h. The solution was concentrated, and saturated NaHCO₃ was added to a pH=7-8. The aqueous layer was extracted with the extract of (40 mL CHCl₃: 5 mL MeOH: 5 mL H₂O) (2×). The combined extracts were dried over Na₂SO₄, filtered, and concentrated to give 221 mg of crude (3-(benzyloxy)-5-isopropylphenyl)methanamine which was used for the next reaction without further purification.

### Example 1.1.17

### N-(3-(aminomethyl)-5-isopropylphenyl)methanesulfonamide

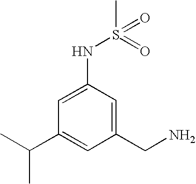

To a stirring solution of 502 mg of 3-amino-5-bromobenzonitrile in 6 mL of CH₂Cl₂ and 2 mL of pyridine at 0° C. was added 0.2 mL (2.57 mmol) of MsCl at 0° C. The ice bath was removed and stirring was continued at r.t. for 6.5 h. The solution was concentrated and EtOAc and 20 mL of H₂O were added. The organic layer was washed with 15 mL of brine and water, dried over Na₂SO₄, filtered, and concentrated. Purification by flash silica gel chromatography (35% EtOAc/hexanes) provided 617 mg of N-(3-bromo-5-cyanophenyl)methanesulfonamide as a colorless solid in 88% yield.

N-(3-cyano-5-(prop-1-en-2-yl)phenyl)methanesulfonamide was synthesized from N-(3-bromo-5-cyanophenyl)methanesulfonamide following the general procedure as described for the Cbz protected Boc aminomethyl bromide.

N-(3-(aminomethyl)-5-isopropylphenyl)methanesulfonamide was synthesized from N-(3-cyano-5-(prop-1-en-2-yl)phenyl)methanesulfonamide following the general procedure as described above for the 3-cyano-5-isopentylpyridine.

### Example 1.1.18

### benzyl 3-(aminomethyl)-5-isopropylphenyl(methyl)carbamate

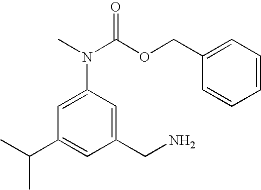

To a stirring solution of 773 mg (2.33 mmol) of benzyl 3-bromo-5-cyanophenylcarbamate in 10 mL of THF at 0° C. was added 231 mg of NaH (60% dispersion in mineral oil). After 1 h, 300 μL (4.82 mmol) of methyl iodide was added, the ice bath was removed, and the red mixture was stirred at r.t. with the light off. After 17 h, saturated NH₄Cl, H₂O, and EtOAc were added and the layers separated. The organic layer was washed with brine, dried over Na₂SO₄, filtered, and concentrated. Purification by flash silica gel chromatography (5% EtOAc/hexanes) provided 195 mg of benzyl 3-bromo-5-cyanophenyl(methyl)carbamate in 24% yield.

benzyl 3-cyano-5-(prop-1-en-2-yl)phenyl(methyl)carbamate was synthesized from benzyl 3-bromo-5-cyanophenyl(methyl)carbamate following the general procedure as described for the Cbz protected Boc aminomethyl bromide.

benzyl 3-(aminomethyl)-5-isopropylphenyl(methyl)carbamate was synthesized from the isopropenylbenzonitrile following the general procedure as described for 3-cyano-5-isopentylpyridine.

### Example 1.1.19

### methyl 3-(aminomethyl)-5-(N-methylmethylsulfonamido)benzoate

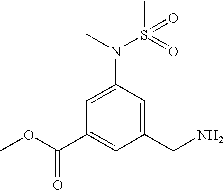

methyl 3-(hydroxymethyl)-5-(N-methylmethylsulfonamido)benzoate was synthesized from 3-(methoxycarbonyl)-5-(N-methylmethylsulfonamido)benzoic acid following the general procedure as described for the 2,6-dimethyl 3,5-pyridyl derivative.

To a stirring solution of 130 mg (0.475 mmol) of methyl 3-(hydroxymethyl)-5-(N-methylmethylsulfonamido)benzoate in 7 mL of toluene and 4 mL of THF was added 120 pt of diphenyl phosphoryl azide (DPPA). The solution was cooled to 0° C. and 86 pt (0.568 mmol) of 1,8-diazabicyclo[5.4.0]-undec-7-ene (DBU) was added. The ice bath was removed, and stirring was continued with warming to room temperature. After about 16 h, the solution was diluted with EtOAc and H₂O, and 1N HCl was added to a pH=8. The organic layer was washed with brine and dried over Na₂SO₄. After filtration and concentration, the crude product was purified by flash silica gel chromatography (50% EtOAc/hexanes) to give 151 mg of methyl 3-(azidomethyl)-5-(N-methylmethylsulfonamido)benzoate as a yellow oil.

The desired product was synthesized from methyl 3-(azidomethyl)-5-(N-methylmethylsulfonamido)benzoate following the general procedure as described for 3-cyano-5-isopentylpyridine.

### Example 1.1.20

### (1-tert-butyl-5-methyl-1H-pyrazol-4-yl)methanamine

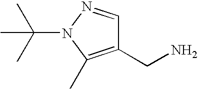

A stirred solution of diethylaminomethylene ethyl acetoacetate (552 mg, 2.8 mmol, see R. A. Fecik, P. Devasthale, S. Pillai, A. Keschavarz-Shokri, L. Sehn, and L. A. Mitscher; J. Med. Chem. 2005, 48, 1229) and tert-butyl hydrazine hydrochloride (387 mg, 3.1 mmol) in EtOH (5 mL) was heated to reflux for 15 h. The reaction mixture was cooled to room temperature and concentrated under reduced pressure. The residue was dissolved in EtOAc and saturated aqueous NaHCO₃. The layers were separated and the aqueous layer was extracted with EtOAc (2×10 mL). The combined organic layer was washed with brine, dried with Na₂SO₄ and concentrated under reduced pressure to provide ethyl 1-tert-butyl-5-methyl-1H-pyrazole-4-carboxylate (560 mg, 96%) as a yellow oil.

4-(azidomethyl)-1-tert-butyl-5-methyl-1H-pyrazole was generated from ethyl 1-tert-butyl-5-methyl-1H-pyrazole-4-carboxylate according to a procedure similar to that described for the 2,6 dimethyl 3,5-pyridyl derivative, then reduced to the crude primary amine, following procedures described herein.

### Example 1.1.21

### (1-(2-methoxyethyl)-5-methyl-1H-pyrazol-4-yl)methanamine

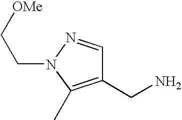

Ethyl 1-(2-hydroxyethyl)-5-methyl-1H-pyrazole-4-carboxylate (376 mg, 67%) was synthesized from diethylaminomethylene ethyl acetoacetate (509 mg, 2.8 mmol) and 2-hydroxyethyl hydrazine (0.30 mL, 4.1 mmol) following the general procedure for ethyl 1-tert-butyl-5-methyl-1H-pyrazole-4-carboxylate as described (see R. A. Fecik, P. Devasthale, S. Pillai, A. Keschavarz-Shokri, L. Sehn, and L. A. Mitscher; J. Med. Chem. 2005, 48, 122). To a stirred solution of NaH (91 mg, 2.3 mmol) in THF (5 mL) was added ethyl 1-(2-hydroxyethyl)-5-methyl-1H-pyrazole-4-carboxylate (376 mg, 1.9 mmol) in THF (1 mL) at 0° C. followed by MeI (0.18 mL, 2.9 mmol). The resulting mixture was stirred for 15 h and quenched with saturated aqueous NH₄Cl. The layers were separated and the aqueous layer was extracted with EtOAc (3×3 mL). The combined organic layer was dried over Na₂SO₄ and concentrated under reduce pressure. The residue was purified by column chromatography to provide ethyl 1-(2-methoxyethyl)-5-methyl-1H-pyrazole-4-carboxylate (249 mg, 62%) as a yellow oil.

4-(azidomethyl)-1-(2-methoxyethyl)-5-methyl-1H-pyrazole was generated from ethyl 1-(2-methoxyethyl)-5-methyl-1H-pyrazole-4-carboxylate according to a procedure similar to that described for the 2,6 dimethyl 3,5-pyridyl derivative, then reduced to the crude primary amine, following procedures described herein.

### Example 1.1.22

### (3-cyclopropylphenyl)methanamine

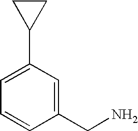

To a solution of tert-butyl 3-bromobenzylcarbamate (572 mg, 2 mmol), cyclopropyl boronic acid (223 mg, 2.6 mmol), potassium phosphate (1.49 g, 7.0 mmol) and tricyclohexyl phosphine (56 mg, 0.2 mmol) in toluene (9 mL) and water (0.45 mL) under a nitrogen atmosphere was added palladium acetate (22 mg, 0.1 mmol). The mixture was heated at 100° C. for 3 h and then cooled to rt. Water (20 mL) was added and the mixture extracted with EtOAc (2×30 mL), the combined organic extracts were washed with brine (20 mL), dried over Na₂SO₄ and concentrated in vacuo. Crude product was purified by column chromatography (15% EtOAc in hexanes) afforded tert-butyl 3-cyclopropylbenzylcarbamate as a colorless oil in 93% yield. (3-cyclopropylphenyl)methanamine was then generated by removing the Boc protecting group by treatment with HCl (in methanol or dioxane) or trifluoroacetic acid in dichloromethane.

### Example 1.1.23

### (5-(prop-1-en-2-yl)pyridin-3-yl)methanamine

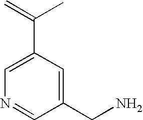

A solution of potassium isopropenyl trifluoroborate (464 mg, 3.13 mmol), PdCl₂(dppf).CH₂Cl₂ (153 mg, 0.06 mmol), tert-butyl (5-bromopyridin-3-yl)methylcarbamate (900 mg, 3.13 mmol) and triethylamine (475 mg, 4.69 mmol) in i-PrOH—H₂O (2:1, 30 mL) was heated under reflux in a nitrogen atmosphere. The reaction mixture was heated at reflux for 4 h, then cooled to rt and diluted with water (40 mL) followed by extraction with diethylether. The organic layers were combined and washed with brine, dried over sodium sulfate and then filtered. The solvent was removed under vacuum, and the crude product was purified by silica gel chromatography (eluting with 45% EtOAc/hexanes) to afford tert-butyl (5-(prop-1-en-2-yl)pyridin-3-yl)methylcarbamate as a white solid in 85% yield. (5-(prop-1-en-2-yl)pyridin-3-yl)methanamine was then generated by removal of the Boc protecting group by treatment with HCl (in methanol or dioxane) or trifluoroacetic acid in dichloromethane.

### Example 1.1.24

### (5-cyclopropylpyridin-3-yl)methanamine

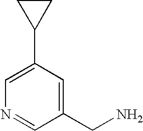

To a solution of tert-butyl (5-bromopyridin-3-yl)methylcarbamate (496 mg, 2 mmol), cyclopropyl boronic acid (223 mg, 2.6 mmol), potassium phosphate (1.49 g, 7.0 mmol) and tricyclohexyl phosphine (56 mg, 0.2 mmol) in toluene (9 mL) and water (0.45 mL) under a nitrogen atmosphere was added palladium acetate (22 mg, 0.1 mmol). The mixture was heated at 100° C. for 3 h and then cooled to rt. Water (20 mL) was added and the mixture extracted with EtOAc (2×30 mL), the combined organic extracts were washed with brine (20 mL), dried over Na₂SO₄ and concentrated in vacuo. Crude product was purified by column chromatography (50% EtOAc in hexanes) afforded tert-butyl (5-cyclopropylpyridin-3-yl)methylcarbamate in 60% yield. (5-cyclopropylpyridin-3-yl)methanamine was then generated by removal of the Boc protecting group by treatment with HCl (in methanol or dioxane) or trifluoroacetic acid in dichloromethane.

### Example 1.1.25

### (4-methoxypyrimidin-2-yl)methanamine

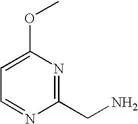

To the 2,4-dichloropyrimidine (1 g, 6.71 mmol) in MeOH (11 mL), NaOMe (362 mg, 6.71 mmol) was added and the reaction mixture was stirred at rt for 0.5 h. Then 15 mL of ether was added and the precipitate was filtered off. Solvent was removed from the reaction mixture and the crude product containing 2-chloro-4-methoxypyrimidine was carried further without any purification.

2-chloro-4-methoxypyrimidine (500 mg, 3.45 mmol) was combined with zinc cyanide (242 mg, 2.07 mmol) and tetrakis (triphenylphosphne)palladium (0) (159 mg, 0.14 mmol) in DMF (10 mL) and the slurry was heated at 80° C. under nitrogen for 6 h. The mixture was cooled to rt, diluted with EtOAc (50 mL) and washed twice with 2N ammonium hydroxide (50 mL). The EtOAc solution was washed with brine (20 mL) and concentrated in vacuo to provide the crude mixture. The crude was then purified by column chromatography (10% EtOAc in hexanes) to afford 4-methoxypyrimidine-2-carbonitrile in 50% yield.

To 4-methoxypyrimidine-2-carbonitrile (370 mg) in MeOH (5 mL), aqueous ammonium hydroxide (1 mL) and Raney Nickel (catalytic) were added and the reaction mixture was hydrogenated at 55 psi for 2 h. Then the reaction mixture was filtered and solvent evaporated to afford (4-methoxypyrimidin-2-yl)methanamine, which was carried to next step without purification.

### Example 1.1.26

### (4-methylpyrimidin-2-yl)methanamine

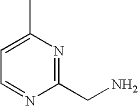

MeMgCl (3M solution in THF, 4.47 mL, 13.42 mmol) was added dropwise to a stirred solution of the 2,4-dichloropyrimidine (2 g, 13.42 mmol) and Fe(acac)₃ (1.37 g, 3.9 mmol) in THF (40 mL) under argon at 0° C. and the resulting reaction mixture was stirred at 0° C. for 8 h. The reaction mixture was diluted with water and extracted with EtOAc. Evaporation of the organic phase followed by column chromatography on a silica gel (eluting with 25% EtOAc/hexanes) to afford 2-chloro-4-methylpyrimidine in 50% yield.

2-chloro-4-methylpyrimidine pyrimidine (725 mg, 5.62 mmol) was combined with zinc cyanide (396 mg, 3.37 mmol) and tetrakis (triphenylphosphne)palladium (0) (716 mg, 0.562 mmol) in DMF (10 mL) and the slurry was heated at 110° C. under nitrogen for 0.5 h. The mixture was cooled to rt, diluted with EtOAc (70 mL) and washed twice with 2N ammonium hydroxide (50 mL). The EtOAc solution was washed with brine (20 mL) and concentrated in vacuo to provide the crude mixture. The crude was then purified by column chromatography (30% EtOAc in hexanes) to afford 4-methylpyrimidine-2-carbonitrile in 67% yield.

4-methylpyrimidine-2-carbonitrile (450 mg) in MeOH (5 mL), aqueous ammonium hydroxide (1 mL) and Raney Nickel (catalytic) were added and the reaction mixture was hydrogenated at 55 psi for 2 h. Then the reaction mixture was filtered and solvent evaporated to afford (4-methylpyrimidin-2-yl)methanamine, which was carried to next step without purification.

### Example 1.1.27

### (5-(trifluoromethyl)pyridin-3-yl)methanamine

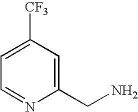

3-bromo-5-(trifluoromethyl)pyridine (1.0 g, 4.42 mmol, 1 eq) was dissolved in 20 mL anhydrous DMF. The solution was degassed by bubbling through with Ar. Zn(CN)₂ (0.312 g, 2.65 mmol, 0.6 eq) and Pd(PPh₃)₄ were added, and the resulting solution was heated to 80° C. with stirring overnight. The reaction was cooled to room temperature and diluted with Et₂O. NH₄OH (28%) was added with stirring and the layers were separated. The organic layer was washed with water (×3), brine (×1), and dried over Na₂SO₄. The inorganics were filtered off, and the reaction mixture was concentrated in vacuo. Purification via flash chromatography on silica gel yielded 0.310 g (1.95 mmol, 44% yield) of 5-(trifluoromethyl)nicotinonitrile.

5-(trifluoromethyl)nicotinonitrile (0.31 g, 1.95 mmol, 1 eq) and CoCl₂.6H₂O (0.23 g, 0.97 mmol, 0.5 eq) were dissolved in 10 mL EtOH. The flask was fitted with a reflux condenser and heated to 50° C. under Ar. NaBH₄ (0.22 g, 5.85 mmol, 3 eq) was added in 2 batches and the mixture was stirred at 50° C. for 2 h. The mixture was then cooled to room temperature and 5 N HCl was added to pH=1-2. The reaction was stirred until the bubbling stopped and NH₄OH (28%) was added to pH=9. The mixture was then concentrated in vacuo and the residue was extracted with CHCl₃/MeOH/water (8:1:1) (×2). The combined extracts were dried over Na₂SO₄. The inorganics were filtered off, and the solvent was removed in vacuo yielding 0.1 g (0.61 mmol, 31% yield) of (5-(trifluoromethyl)pyridin-3-yl)methanamine.

### Example 1.1.28

### 3-(aminomethyl)-N,N-dimethylaniline

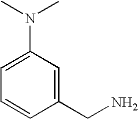

(Boc)₂O (1.06 mL, 1.0 g, 4.62 mmol, 1.1 eq) was added to a stirred solution of 3-(aminomethyl)aniline (0.512 g, 4.2 mmol, 1 eq) and Et₃N (1.3 mL, 0.94 g, 9.24 mmol, 2.2 eq) in 5 mL anhydrous MeOH at 0° C. under Ar. The solution was stirred at 0° C. to room temperature overnight. The solvent was removed in vacuo. The residue was dissolved in EtOAc, washed with water (×2), brine (×1), and dried over Na₂SO₄. The inorganics were filtered off, and the solvent was removed in vacuo. Purification via flash chromatography on silica gel yielded 0.8746 g (3.9 mmol, 94% yield) of tert-butyl 3-aminobenzylcarbamate.

CH₂O (aq. 37%, 1.57 mL, 21.1 mmol, 10 eq) was added to a stirred solution of tert-butyl 3-aminobenzylcarbamate (0.47 g, 2.1 mmol, 1 eq) in 10 mL CH₃CN. The resulting solution was treated with NaBH₃CN (0.42 g, 6.3 mmol, 3 eq) followed by the dropwise addition of HOAc to pH=7. The solution was stirred for 1 h adding HOAc occasionally to keep the pH close to 7. The reaction was concentrated in vacuo and the resulting residue was diluted with sat. NaHCO₃ and extracted into EtOAc (×1). The organic layer was washed with water (×3), brine (×1), and dried over Na₂SO₄. The inorganics were filtered off, and the solvent removed in vacuo. Purification via flash chromatography on silica gel yielded 0.39 g (1.6 mmol, 74% yield) of tert-butyl 3-(dimethylamino)benzylcarbamate.

3-(aminomethyl)-N,N-dimethylaniline was then generated by removal of the Boc protecting group by treatment with HCl (in methanol or dioxane) or trifluoroacetic acid in dichloromethane.

### Example 1.1.29

### N-methyl-1-(5-methylthiazol-2-yl)methanamine

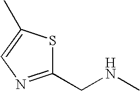

To a solution of BuLi (1.6 M in hexanes, 12.6 mL, 20.2 mmol) in 25 mL of diethyl ether at −78° C., was added a solution of 5-methylthiazole (2.0 g, 20.2 mmol) in ether (6 mL), drop-wise and stirred at −78° C. for 1.5 h. A solution of DMF (2.33 mL, 30.3 mmol in ether (5 mL) was added at once and the reaction mixture was allowed to warm to room temperature and stirred overnight. Ice was added to the reaction mixture followed by the slow addition of 4N HCl. The mixture was taken up in a separating funnel, ether added (30 mL) and shaken. The organic layer was discarded. The aqueous layer was brought to pH ˜7.5 with solid NaHCO₃ and extracted with ether twice. The ether layer was dried over Na₂SO₄ and concentrated, and the resulting crude 5-methylthiazole-2-carbaldehyde (1.6 g) was carried over to the next-step without purification.

Ti (OⁱPr)₄ (1.3 eq) was added with stirring to MeNH₂ (2.0 M in MeOH, 3 eq) under Ar. After 5 min. 5-methylthiazole-2-carbaldehyde (1 eq) was added, and the solution was stirred for 1-2 h. The reaction was cooled to 0° C. and NaBH₄ (1.3 eq) was added. The solution was stirred at 0° C. to room temperature overnight. After quenching the reaction with water the mixture was filtered through Celite to remove the white ppt. MeOH was removed in vacuo and the residue was diluted with EtOAc. The resulting solution was washed with water (×3), brine (×1), and dried over Na₂SO₄. The inorganic material was filtered off, and the solvent was removed in vacuo to give the crude product. Purification via column chromatography yielded N-methyl-1-(5-methylthiazol-2-yl)methanamine in 80-85% yield.

### Example 1.1.30

### N-((4-methylthiazol-2-yl)methyl)ethanamine

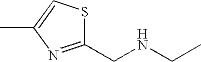

N-((4-methylthiazol-2-yl)methyl)ethanamine was prepared following a similar procedure as N-methyl-1-(5-methylthiazol-2-yl)methanamine using EtNH₂.

### Example 1.1.31

### N-((4-methylthiazol-2-yl)methyl)propan-1-amine

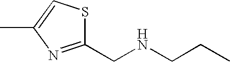

N-((4-methylthiazol-2-yl)methyl)propan-1-amine was prepared following a similar procedure as N-methyl-1-(5-methylthiazol-2-yl)methanamine using n-propylamine.

### Example 1.1.32

### N-((4-methylthiazol-2-yl)methyl)propan-2-amine

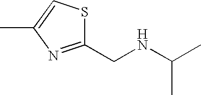

N-((4-methylthiazol-2-yl)methyl)propan-2-amine was prepared following a similar procedure as N-methyl-1-(5-methylthiazol-2-yl)methanamine using isopropylamine.

### Example 1.1.33

### N-((4-methylthiazol-2-yl)methyl)cyclopropanamine

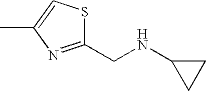

N-((4-methylthiazol-2-yl)methyl)cyclopropanamine was prepared following a similar procedure as N-methyl-1-(5-methylthiazol-2-yl)methanamine using cyclopropanamine.

### Example 1.1.34

### 1-(4,5-dimethylthiazol-2-yl)-N-methylmethanamine

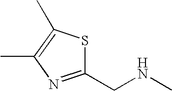

1-(4,5-dimethylthiazol-2-yl)-N-methylmethanamine was prepared following a similar procedure as N-methyl-1-(5-methylthiazol-2-yl)methanamine from 4,5-dimethylthiazole-2-carboxyaldehyde.

### Example 1.1.35

### 2-((4-methylthiazol-2-yl)methylamino)ethanol

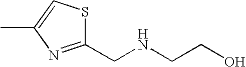

Ti(OⁱPr)₄ (3.4 mL, 3.3 g, 11.7 mmol, 1.3 eq) was added to a stirred solution of ethanolamine in 10 mL anhydrous MeOH under Ar. After 10 min, 4-methylthiazole-2-carbaldehyde (1 mL, 1.18 g, 9 mmol, 1 eq) was added. After 1 h the reaction was cooled to 0° C. and the NaBH₄ (0.44 g, 11.7 mmol, 1.3 eq) was added. The reaction was stirred at 0° C. to room temperature overnight. The reaction was quenched with water, and a white ppt formed. The mixture was filtered through Celite, and the MeOH removed in vacuo. The residue was diluted with EtOAc, washed with water (×3), brine (×1), and dried over Na₂SO₄. The inorganics were filtered off, and the solvent was removed in vacuo yielding 1.14 g (7.2 mmol, 80% yield) of the crude product 2-((4-methylthiazol-2-yl)methylamino)ethanol, which was used without further purification.

### Example 1.1.36

### (3-methylisoxazol-5-yl)methanamine

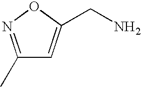

A solution of 321 mg (2.27 mmol) of 2-(3-methylisoxazol-5-yl)acetic acid, 0.5 mL (2.32 mmol) of diphenylphosphorylazide (DPPA), and 0.35 mL (2.51 mmol) of triethylamine in 30 mL of distilled tert-butyl alcohol was refluxed for 13.5 h. The solution was concentrated, and the crude residue was dissolved in EtOAc. The organic layer was washed with 1N HCl (3×10 mL) and saturated NaHCO₃ solution (3×10 mL). The organic layer was dried over sodium sulfate, filtered, concentrated. Purification by flash silica gel chromatography (28% EtOAc/hexanes) provided 50 mg (10% yield) of the protected amine as a pale yellow solid. (3-methylisoxazol-5-yl)methanamine was then generated by removal of the Boc protecting group by treatment with HCl (in methanol or dioxane) or trifluoroacetic acid in dichloromethane.

### Example 1.1.37

### (3-(methoxymethyl)phenyl)methanamine

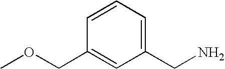

1,3-phenylenedimethanol was converted to (3-(methoxymethyl)phenyl)methanol using the procedure found in the following reference: Liu, Xuan; Zheng, Qi-Huang; Fei, Xiangshu; Wang, Ji-Quan; Ohannesian, David W.; Erickson, Leonard C.; Stone, K. Lee; Hutchins, Gary D.; Bioorg. Med. Chem. Lett. 2003, 13, 641-644. (3-(methoxymethyl)phenyl)methanol was converted to the target molecule following standard reactions including formation of the azide with DPPA and reduction.

### Example 1.1.38

### N-(3-(aminomethyl)phenyl)acetamide

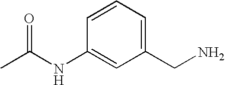

To a stirred solution of tert-butyl 3-aminobenzylcarbamate (Hah, Jung-Mi; Martasek, Pavel; Roman, Linda J.; Silverman, Richard B.; J. Med. Chem.; 2003, 46, 1661-1669) (287 mg, 1.29 mmol) in CH₂Cl₂ (10 mL) at 0° C. was added Et₃N (0.27 mL, 2.0 mmol) and acetyl chloride (0.10 mL, 1.4 mmol) and the resulting solution was warmed up to room temperature slowly. After further stirring of 4 h, the reaction was quenched with saturated aqueous NH₄Cl. The layers were separated and the aqueous layer was extracted with CH₂Cl₂ (2×20 mL). The combined organic layer was washed with H₂O, brine, dried with Na₂SO₄ and concentrated under reduced pressure. The residue oil was purified by column chromatography (60% EtOAc in hexanes) to provide tert-butyl 3-acetamidobenzylcarbamate (123.1 mg, 36%). N-(3-(aminomethyl)phenyl)acetamide was then generated by removal of the Boc protecting group by treatment with HCl (in methanol or dioxane) or trifluoroacetic acid in dichloromethane.

### Example 1.1.39

### 3-(aminomethyl)-N-methylaniline

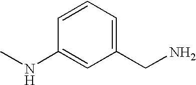

Tert-butyl 3-aminobenzylcarbamate was converted to tert-butyl 3-(methylamino)benzylcarbamate following standard reductive amination conditions using formaldehyde and sodium cyanoborohydride. 3-(aminomethyl)-N-methylaniline was then generated by removal of the Boc protecting group by treatment with HCl (in methanol or dioxane) or trifluoroacetic acid in dichloromethane.

### Example 1.1.40

### (5-(benzyloxy)pyridin-3-yl)methanamine

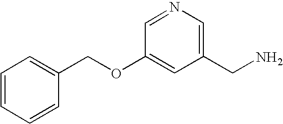

A mixture of 818 mg (5.34 mmol) of 5-hydroxynicotinic acid, 1.70 g (12.3 mmol) of K₂CO₃, and 1.0 mL (8.41 mmol) of benzyl bromide in 25 mL of DMF was heated at 60° C. under Ar for 16 h. The mixture was filtered through cotton, and the residue was dissolved in CHCl₃. The organic layer was washed with water (2×30 mL), brine (30 mL), dried over Na₂SO₄, filtered, and concentrated. Purification by flash silica gel chromatography provided 363 mg of methyl 5-(benzyloxy)nicotinate in 28% yield as an orange oil.

To a stirring solution of 363 mg (1.49 mmol) of methyl 5-(benzyloxy)nicotinate in 10 mL of THF at 0° C. was added 141 mg (3.71 mmol) of LiAlH₄. The ice bath was removed, and after 55 min., 20.5 mg of LiAlH₄ was added. After 40 min., the reaction was quenched by adding successively 160 μL of H₂O, 160 μL of 15% aqueous NaOH, and 480 μL of brine. Purification by flash silica gel chromatography (2 mL MeOH/100 mL CHCl₃) provided 250 mg of (5-(benzyloxy)pyridin-3-yl)methanol (yellow oil) in 78% yield.

To a stirring solution of 250 mg (1.17 mmol) of (5-(benzyloxy)pyridin-3-yl)methanol in 8 mL of toluene was added 310 μL (1.44 mmol) of DPPA. The mixture was cooled to 0° C. and 210 μL (1.44 mmol) of 1,8-diazabicyclo[5.4.0]-undec-7-ene] (DBU) was added. The ice bath was removed, and stirring was continued with warming to room temperature. After about 20 h, the solution was diluted with EtOAc, and 1N HCl was added to a pH between 7 and 8. The organic layer was washed with water (2×15 mL) and brine (15 mL) and dried over Na₂SO₄. After filtration and concentration, the crude product was purified by flash silica gel chromatography (56-60% EtOAc/hexanes) to give 181 mg (65% yield) of 3-(azidomethyl)-5-(benzyloxy)pyridine as a colorless oil.

To a stirring solution of 181 mg of 3-(azidomethyl)-5-(benzyloxy)pyridine in 6 mL of THF at 0° C. was added 80.6 mg (2.12 mmol) of LiAlH₄. The ice bath was removed and stirring was continued with warming to r.t. After 30 min. the reaction was quenched by adding successively 160 μL of H₂O, 160 μL of 15% aqueous NaOH, and 480 μL of brine. The mixture was filtered through Celite and concentrated. The crude product was used for the next reaction without further purification.

### Example 1.1.41

### (5-methylpyridin-3-yl)methanamine

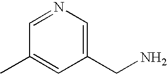

To stirring solution of 233 mg (1.70 mmol) of 5-methylnicotinic acid (synthesized following the general procedure for 5-fluoro-isophthalic acid) in 30 mL of THF at 0° C. was added 181 mg (4.76 mmol) of LiAlH₄. After 25 min., the reaction was quenched by adding successively 180 μL of H₂O, 180 μL of 15% aqueous NaOH, and 540 μL of brine. The mixture was filtered through Celite and concentrated to give 87 mg of (5-methylpyridin-3-yl)methanol which was used for the next reaction without further purification.

(5-methylpyridin-3-yl)methanamine was synthesized from (5-methylpyridin-3-yl)methanol following the general procedure as described for the nicotinic acid benzyl ether derivative.

### Example 1.1.42

### (6-methylpyridin-2-yl)methanamine

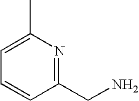

Diphenylphosphoryl azide (DPPA) (0.74 mL, 0.9 g, 3.4 mmol, 1.2 eq) and 1,8-Diazabicyclo(5.4.0)undec-7-ene (DBU) (0.508 mL, 0.52 g, 3.4 mmol, 1.2 eq) were added to a stirred solution of the (6-methylpyridin-2-yl)methanol (0.35 g, 2.8 mmol, 1 eq) in 7 mL anh. toluene under Ar. After stirring overnight, the solvent was removed in vacuo. Purification via flash chromatography yielded 0.47 g (3.2 mmol, 113% yield of the crude product. The crude was dissolved in 5 mL MeOH. Pd(OH)₂ (20% by wt. on carbon, 0.040 g) was added, and the mixture was stirred vigorously under H₂ overnight. The mixture was filtered through Celite, and the filter cake rinsed with MeOH. The solvent was removed in vacuo yielding 0.4948 g of crude (6-methylpyridin-2-yl)methanamine.

### Example 1.1.43

### (5-methoxypyridin-3-yl)methanamine

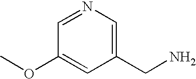

(5-methoxypyridin-3-yl)methanamine was synthesized from the hydroxynicotinate following the general procedure as described for the nicotinic acid benzyl ether derivative.

### Example 1.1.44

### (1H-indol-7-yl)methanamine

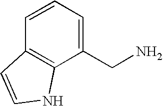

1H-indole-7-carbaldehyde (1 g, 6.9 mmol) in EtOH (30 mL), hydroxylamine (527 mg, 7.6 mmol) in water (10 mL) was added followed by 50% NaOH (1.38 g in 1.38 mL water) was added. After refluxing for 2 h, ethanol was removed under reduced pressure. Resultant slurry was extracted with ethylacetate. Organic layer was washed with water, brine and dried. Crude residue was column chromatographed on a silica gel eluting with (30% EtOAc/hexanes) to afford (Z)-1H-indole-7-carbaldehyde oxime in 80% yield.

(Z)-1H-indole-7-carbaldehyde oxime (100 mg, 0.60 mmol) in MeOH (5 mL), Pd(OH)₂ (50 mg) was added and stirred under hydrogen atmosphere (balloon pressure) for 4 h. Reaction mixture was then filtered and solvent evaporated to yield (1H-indol-7-yl)methanamine in quantitative yield.

### Example 1.1.45

### (3-tert-butylphenyl)methanamine

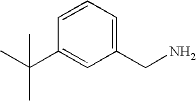

To 3-tert-butylphenol (3 g, 20 mmol) in pyridine (11 mL, 140 mmol) at 0° C., triflic anhydride (4.06 mL, 24 mmol) was added and stirred at 0° C. for 1 h. Then the reaction mixture was allowed to come to rt and stirred at rt for 4 h. Then the reaction mixture was diluted with ether, washed with water. Organic layers were collected washed with dilute HCl, water, brine and dried over anhydrous sodium sulfate. Volatiles were removed on a rotavap under reduced pressure. The crude 3-tert-butylphenyl trifluoromethanesulfonate was carried to the next step without any further purification.

To a solution of 3-tert-butylphenyl trifluoromethanesulfonate (1.5 g, 6.70 mmol) in DMF (10 mL), zinc cyanide (1.57 g, 13.40 mmol) was added. The reaction mixture was heated to 120° C. for 4 h. Then reaction mixture was cooled, diluted with ether (2500 mL) and washed twice with 2N ammonium hydroxide (50 mL). The ether solution was washed with brine (20 mL) and concentrated in vacuo to provide the crude mixture. The crude was then purified by column chromatography (5% EtOAc in hexanes) to afford 3-tert-butylbenzonitrile in 75% yield.

To 3-tert-butylbenzonitrile (400 mg) in MeOH (5 mL), aqueous ammonium hydroxide (1 mL) and Raney Nickel (catalytic) were added and the reaction mixture was hydrogenated at 50 psi for 2 h. Then the reaction mixture was filtered and solvent evaporated. (3-tert-butylphenyl)methanamine was used without any purification.

### Example 1.1.46

### 2-(aminomethyl)-6-tert-butylphenol

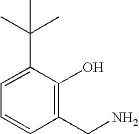

To a stirred solution of hydroxylamine hydrochloride (437 mg, 6.3 mmol) in CH₃CN at 0° C. was added Et₃N and 3-tert-butyl-2-hydroxybenzaldehyde (1.02 g, 5.7 mmol). The reaction mixture was warmed up to room temperature and stirred for 24 h. The solvent was removed and the residue was dissolved in EtOAc and H₂O. The layers were separated and the aqueous layer was extracted with EtOAc (2×20 mL). The combined organic layer was washed with brine, dried with Na₂SO₄ and concentrated under reduced pressure to provide 3-tert-butyl-2-hydroxybenzaldehyde oxime (1.01 g, 92%) as a pale yellow solid.

3-tert-butyl-2-hydroxybenzaldehyde oxime (550 mg, 2.9 mmol) in EtOAc (10 mL) and MeOH (10 mL) was hydrogenated at balloon pressure in the presence of 10% Pd on carbon (100 mg) for 15 h. The reaction mixture was filtered over celite and concentrated. The residue was dissolved in EtOAc and 0.5 N HCl (10 mL). The layers were separated and the aqueous layer was treated with 1N NaOH solution until pH=9. The resulting aqueous layer was extracted with CHCl₃ (3×10 mL). The combined CHCl₃ was washed with brine, dried with Na₂SO₄ and concentrated under reduced pressure to provide to desired product (123 mg, 23%).

### Example 1.1.47

### (5-tert-butyl-2-(tert-butyldimethylsilyloxy)phenyl)methanamine

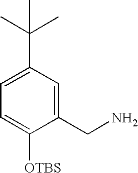

To a stirred solution of 5-tert-butyl-2-hydroxybenzaldehyde (570 mg, 3.2 mmol) in DMF (5 mL) was added imidazole (435 mg, 6.4 mmol) and TBSC1 (576 mg, 3.8 mmol). The reaction mixture was stirred for 15 h and quenched with saturated aqueous NH₄Cl. The resulting mixture was extracted with EtOAc (2×20 mL). The combined organic layer was washed with H₂O, brine, dried with Na₂SO₄ and concentrated under reduced pressure to provide 5-tert-butyl-2-(tert-butyldimethylsilyloxy)benzaldehyde (1.07 g, quantitative).

(5-tert-butyl-2-(tert-butyldimethylsilyloxy)phenyl)methanamine was generated from 5-tert-butyl-2-(tert-butyldimethylsilyloxy)benzaldehyde using a similar procedure to the described synthesis of 2-(aminomethyl)-6-tert-butylphenol.

### Example 1.1.48

### (2-fluoro-6-methoxyphenyl)methanamine

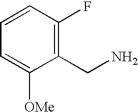

(2-fluoro-6-methoxyphenyl)methanamine was synthesized from 2-fluoro-6-methoxybenzaldehyde using a similar procedure to the described synthesis of 2-(aminomethyl)-6-tert-butylphenol.

### Example 1.1.49

### (3-(2-methyl-1,3-dioxolan-2-yl)phenyl)methanamine

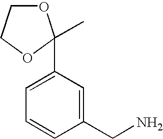

A stirred solution of 3-acetylbenzonitrile (1.05 g, 7.2 mmol), ethylene glycol (0.81 mL, 14.5 mmol) and TsOH (0.65 g, 7.2 mmol) was heated at reflux with a Dean-Stark for 15 h during which time the reaction became dark brown suspension. The reaction mixture was cooled to room temperature and diluted with saturated aqueous NaHCO₃. The layers were separated and the aqueous layer was extracted with EtOAc (2×20 mL). The combined organic layer was washed with brine, dried with Na₂SO₄ and concentrated under reduced pressure. The residue was purified by column chromatography (10% EtOAc in hexanes) to provide 3-(2-methyl-1,3-dioxolan-2-yl)benzonitrile (1.08 g, 79%).

To a stirred solution of LiAlH₄ (114 mg, 3.6 mmol) in ether at 0° C. was added 3-(2-methyl-1,3-dioxolan-2-yl)benzonitrile (0.57 g, 3.0 mmol). The reaction mixture was stirred for 4 h and quenched with H₂O (0.2 mL), 20% NaOH (0.2 mL), brine (0.6 mL). The resulting mixture was stirred for 1 h and filtered over celite and concentrated. The residue was purified by column chromatography (10% MeOH in CHCl₃) to provide (3-(2-methyl-1,3-dioxolan-2-yl)phenyl)methanamine (334 mg, 57%).

The ethylene ketal protecting group can be removed following amide coupling using standard conditions known in the art to generate the desired ketone derivative.

### Example 1.1.50

### (R)-1-(4-methyloxazol-2-yl)ethanamine

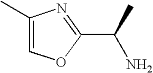

To a solution of L-Serine methyl ester hydrochloride (5.0 g, 32.0 mmol), in CH₂Cl₂ (150 mL) at 0° C., were added Et₃N (4.88 mL, 35.2 mmol), Boc-D-Alalnine (6.06 g, 32 mmol), and DCC (7.26 g, 35.2 mmol) sequentially. The reaction was allowed to warm to room temperature and stirred overnight. All the solvent was evaporated and the residue was triturated with ethyl acetate and the precipitate was filtered off. The filtrate was concentrated under low pressure and chromatographed on silica gel (70% ethyl acetate/30% chloroform) to yield 86% of methyl 2-((R)-2-(tert-butoxycarbonylamino)propanamido)-3-hydroxypropanoate.

Deoxo-fluor™ (Bis-(2-methoxy)amino sulfur trifluoride, 1.4 mL, 7.6 mmol) was added drop-wise to a solution of methyl 2-((R)-2-(tert-butoxycarbonylamino)propanamido)-3-hydroxypropanoate (2.0 g, 6.9 mmol) in CH₂Cl₂ (50 mL) at −20° C. The solution was stirred for 30 min and BrCCl₃ (2.45 mL, 24.8 mmol) was added drop-wise. The reaction was stirred at 2-3° C., for 8 h., quenched with sat. aq. NaHCO₃ solution and extracted with ethyl actetate. The organic layer was concentrated and chromatographed on silica gel (30% ethyl acetate/70% hexanes) to yield 65% of (R)-methyl 2-(1-(tert-butoxycarbonylamino)ethyl)oxazole-4-carboxylate.

To a solution of (R)-methyl 2-(1-(tert-butoxycarbonylamino)ethyl)oxazole-4-carboxylate (3.07 g, 11.37 mmol) in THF (25 mL) at 0° C., was added LiBH₄ (17.0 mL, 2.0M in THF, 34.0 mmol). The reaction was allowed to warm to room temperature and stirred for 3 h. Ethyl acetate (11 mL) was added drop-wise and stirred for 30 min. The reaction was cooled to 0° C. and 17 mL of 1N HCl was added drop-wise and diluted with 30 mL of water. The mixture was then extracted with ethyl acetate, dried on Na₂SO₄, concentrated, and chromatographed on silica gel (3% MeOH/97% chloroform) to yield 84% of (R)-tert-butyl 1-(4-(hydroxymethyl)oxazol-2-yl)ethylcarbamate.

To a solution of TPP (873 mg, 3.33 mmol) in CH₂Cl₂ (10 mL), was added I₂ (845 mg, 3.33 mmol), and stirred for 10 min. Imidazole (227 mg, 3.33 mmol) was added and stirred for an additional 10 min and then a solution of (R)-tert-butyl 1-(4-(hydroxymethyl)oxazol-2-yl)ethylcarbamate (537 mg, 2.22 mmol) in CH₂Cl₂ (15 mL) was added. After 2 h, the reaction mixture was washed successively with sat. aq. NaHCO₃, aq. Na₂S₂O₃, dried on Na₂SO₄ and concentrated under low pressure. The residue was chromatographed on silica gel (20% ethyl acetate/80% hexanes) to yield 84% of (R)-tert-butyl 1-(4-(iodomethyl)oxazol-2-yl)ethylcarbamate.

To a solution of (R)-tert-butyl 1-(4-(iodomethyl)oxazol-2-yl)ethylcarbamate (660 mg, 1.87 mmol) in HMPA (10 mL), was added NaCNBH₃ (470 mg, 7.5 mmol). The reaction was stirred for 4 h. and poured into ice-cold water and extracted with hexanes. The organic layer was dried on Na₂SO₄, concentrated, and chromatographed on silica gel (10% ethyl acetate/90% hexanes) to yield 38% of (R)-tert-butyl 1-(4-methyloxazol-2-yl)ethylcarbamate.

(R)-1-(4-methyloxazol-2-yl)ethanamine was then generated by removal of the Boc protecting group by treatment with HCl (in methanol or dioxane) or trifluoroacetic acid in dichloromethane.

### Example 1.1.51

### (4-methyloxazol-2-yl)methanamine

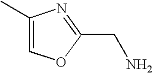

(4-methyloxazol-2-yl)methanamine was generated using a procedure similar to the synthesis of (R)-1-(4-methyloxazol-2-yl)ethanamine using Boc-glycine as starting material.

### Example 1.1.52

### (5-isopropylpyridin-3-yl)methanamine

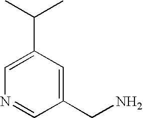

Methyl 5-bromonicotinate was reduced to (5-bromopyridin-3-yl)methanol using LiAlH₄ under conditions well known in the art. (5-bromopyridin-3-yl)methanol was transformed to (5-isopropylpyridin-3-yl)methanamine similar to the procedures described for methyl 3-(aminomethyl)-5-(N-methylmethylsulfonamido)benzoate and benzyl 3-(aminomethyl)-5-isopropylphenyl(methyl)carbamate.

### Example 1.1.53

### 5-(aminomethyl)-N,N-dimethylpyridin-3-amine

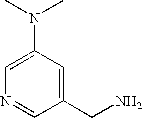

5-methyl 5-aminonicotinate was generated from pyridine-3,5-dicarboxylic acid following procedures well know in the art and synthesis described herein. To a stirring solution of 5-methyl 5-aminonicotinate (283 mg, 1.86 mmol) in 19 mL of CH₃CN and 19 mL of 37% formaldehyde in H₂O was added 353 mg (5.62 mmol) of NaCNBH₃, and 50 drops of acetic acid. The solution was stirred at r.t. for 18.5 h and 40 mL of EtOAc and 40 mL of sat. NaHCO₃ solution were added. The layers were separated, and the organic layer was washed with 25 mL of sat. NaHCO₃ and 25 mL of brine, dried over Na₂SO₄, filtered, and concentrated. Purification by flash silica gel chromatography (0.5% MeOH/CHCl₃) resulted in 124 mg of methyl 5-(dimethylamino)nicotinate as a yellow oil in 37% yield.

Methyl 5-(dimethylamino)nicotinate was then transformed to 5-(aminomethyl)-N,N-dimethylpyridin-3-amine using a procedure similar to the synthesis of (5-isopropylpyridin-3-yl)methanamine described herein.

### Example 1.1.54

### (6-(trifluoromethyl)pyridin-3-yl)methanamine

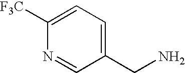

A mixture of (6-(trifluoromethyl)pyridin-3-yl)methanol (2.0 g, 11.3 mmol) and diphenyl phoephorazidate (2.93 mL, 14 mmol) was dissolved in dry toluene (20 mL). The mixture was cooled to 0° C. under Argon, and neat DBU (2.1 mL, 14 mmol) was added. The reaction mixture was stirred for 2 h at 0° C. and then at rt for 16 h. The resulting two-phase mixture was washed with water and extracted with EtOAc. The combined organic layer was concentrated in vacuo and purified by silica gel chromatography afford 5-(azidomethyl)-2-(trifluoromethyl)pyridine (2.3 g, quantative yield) of a light yellow oil: ¹H NMR (300 MHz, CDCl₃+CD₃OD), d: 8.686 (m, 1H), 7.853 (m, 1H), 7.723 (d, J=8.1 Hz, 1H), 4.518 (s, 2H).

5-(azidomethyl)-2-(trifluoromethyl)pyridine (2.6 g, 12.86 mmol) in THF at −78° C. was added LAH (0.54 g, 14.2 mmol). The resulting mixture was stirred for 15 min. and warmed to room temperature for one hour. Then the reaction was quenched with saturated aqueous NH₄Cl and stirred for a couple of hours. Anhydrous Na₂SO₄ was added to make the mixture clear two phases, filtered and washed with EtOAc. The combined organic solution was concentrated to provide (6-(trifluoromethyl)pyridin-3-yl)methanamine as a red syrup (2.1 g). ¹H NMR (300 MHz, CDCl₃+CD₃OD), d: 8.729 (s, 1H), 7.928 (d, J=9.1 Hz, 1H), 7.706 (d, J=9.1 Hz, 1H), 4.054 (s, 2H).

### Example 1.1.55

### N-methyl-1-(4-methylthiazol-2-yl)methanamine

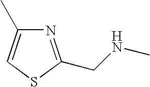

Ti(OⁱPR)₄ (1.3 eq) was added with stirring to MeNH₂ (2.0 M in MeOH, 3 eq) at 0° C. under Ar. After 15 min. 4-methylthiazole-2-carbaldehyde (1 eq) was added, and the solution was stirred for 2-3 h. NaBH₄ (1.4 eq, in batches if large scale) was added and stirred at 0° C. to RT overnight, followed by solvent removal in vacuo. The residue was diluted with water/CH₂Cl₂, and a white ppt formed. The mixture was then filtered through Celite to remove the white ppt and the layers were separated. The aqueous layer was extracted with CH₂Cl₂ (×3) and the combined organics were dried over Na₂SO₄. The inorganics were filtered off, and the solvent was removed in vacuo to give the crude product. Purification via column chromatography yielded the pure product in 80-90% yield.

### Example 1.1.56

### (3-methyl-1,2,4-oxadiazol-5-yl)methanamine

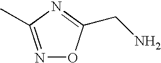

To a stirred solution of acetonitrile (5 mL, 95 mmol) in a 4:1 mixture of EtOH and water (180 mL) were added NaOH (4.26 g, 107 mmol) and hydroxylamine hydrochloride (7.1 g, 0.1 mmol) and the reaction was refluxed for 24 h. It was then concentrated under reduced pressure. The white solid was dissolved in 150 mL of absolute EtOH and filtered to remove the inorganic salts. Concentration of the filtrate gave a crude white solid, which was recrystallized form isopropanol to obtain 3.2 g of (Z)—N′-hydroxyacetimidamide.

To a stirred suspension of molecular sieves 3A° in anhydrous THF (200 mL) was added (Z)—N′-hydroxyacetimidamide (900 mg, 10.0 mmol) and stirred for 15 min. NaH (1.3 g, 32.0 mmol) wad added and the reaction was stirred for 45 min. Then Glycine methyl ester (1.89 g, 10 mmol) was added and the reaction was refluxed overnight, cooled filtered over celite and concentrated. The residue was dissolved in CH₂Cl₂, washed with water, dried on Na₂SO₄ and concentrated. The residue was purified by column chromatography (40% EtOAc in hexanes) to provide 460 mg of tert-butyl (3-methyl-1,2,4-oxadiazol-5-yl)methylcarbamate.

tert-butyl (3-methyl-1,2,4-oxadiazol-5-yl)methylcarbamate was converted into (3-methyl-1,2,4-oxadiazol-5-yl)methanamine using standard deprotection protocol of Boc group with TFA.

### Example 1.1.57

### (5-methyl-1,2,4-oxadiazol-3-yl)methanamine

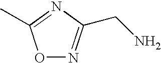

To a stirred solution of N-Boc-2-aminoacetonitrile (3.0 g, 19.21 mmol) in a 4:1 mixture of EtOH and water (25 mL) were added NaOH (860 mg, 21.5 mmol) and hydroxylamine hydrochloride 9 1.44 g, 20.7 mmol) and the reaction was stirred for 30 h. All the solvent was evaporated under reduced pressure. The solid was dissolved in water and the aqueous layer was extracted with EtOAc. The combined organic layers were dried on Na₂SO₄ and concentrated to provide 1.8 g of (Z)-tert-butyl 2-amino-2-(hydroxyimino)ethylcarbamate.

To a stirred solution of (Z)-tert-butyl 2-amino-2-(hydroxyimino)ethylcarbamate (945 mg, 5 mmol) and EtOAc (2.0 mL, 20.0 mmol) in EtOH (100 mL) was added a solution of NaOEt in EtOH (13 mL, 50.0 mmol) and refluxed for 6 h. The reaction mixture was cooled and all the solvent was evaporated under reduced pressure. The residue was dissolved in water and the aqueous layer was extracted with EtOAc. The combined organic layers were dried on Na₂SO₄ and concentrated to provide 1.0 g of tert-butyl (5-methyl-1,2,4-oxadiazol-3-yl)methylcarbamate.

tert-butyl (5-methyl-1,2,4-oxadiazol-3-yl)methylcarbamate was converted into (5-methyl-1,2,4-oxadiazol-3-yl)methanamine using standard deprotection protocol of Boc group with TFA.

### Example 1.1.58

### 3-(aminomethyl)-N,N-diethylaniline

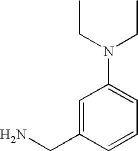

Acetaldehyde (0.52 ml, 0.4 g, 9.08 mmol, 5 eq) was added to a stirred solution of tert-butyl 3-aminobenzylcarbamate (derived from 3-(aminomethyl)aniline (TCI America)) in 11 ml CH₃CN/H₂O (10:1) at 0° C. After 5 min NaBH₃CN (0.3 g, 4.54 mmol, 2.5 eq) was added. The reaction was adjusted to pH ^{˜}7 with HOAc and stirred at 0° C. for 5 min. The ice-bath was removed and the reaction was stirred at room temperature for 45 min. The solvent was removed in vacuo. The residue was diluted with saturated aqueous NaHCO₃ and extracted with EtOAc (×2). The combined organics were washed with water (×3), brine (×1), and dried over Na₂SO₄. The inorganics were filtered off, and the solvent was removed in vacuo. Purification via flash chromatography yielded 0.3875 g (1.4 mmol, 77% yield) of tert-butyl 3-(diethylamino)benzylcarbamate.

MeOH.HCl (1.25 M, 11 ml, 13.9 mmol, 10 eq) was added to a flask charged with tert-butyl 3-(diethylamino)benzylcarbamate (0.3875 g, 1.4 mmol, 1 eq) at 0° C. under Ar. After stirring for 1.5 h at 0° C. the ice-bath was removed. After stirring at room temperature for 6 h, the solvent was removed in vacuo. The residue was stirred with saturated aqueous NaHCO₃ for 30 min and extracted with CH₂Cl₂ (×2). The combined organics were dried over Na₂SO₄. The inorganics were filtered off, and the solvent was removed in vacuo to yield 0.191 g (1.07 mmol, 77% yield) of the product.

### Example 1.1.59

### 3-(aminomethyl)-N-methylaniline

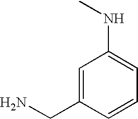

A stirred solution of the starting tert-butyl 3-aminobenzylcarbamate (0.925 g, 4.16 mmol, 1 eq, derived from 3-(aminomethyl)aniline (TCI America)) in 10 ml anhydrous DMF under Ar was treated with benzyl bromide (0.54 ml, 0.78 g, 4.58 mmol, 1.1 eq) and Et₃N (0.75 ml, 0.55 g, 1.3 eq). After stirring for 48 h the reaction was diluted with water and extracted with EtOAc (×2). The combined organics were washed with water (×4), brine (×1), and dried over Na₂SO₄. The inorganics were filtered off, and the solvent was removed in vacuo. Purification via flash chromatography yielded 0.3937 g (1.26 mmol, 30% yield) of tert-butyl 3-(benzylamino)benzylcarbamate.

CH₂O (aq, 37%) (0.112 ml, 0.12 g, 1.51 mmol, 2 eq) was added to a stirred solution of tert-butyl 3-(benzylamino)benzylcarbamate (0.2353 g, 0.753 mmol, 1 eq) in 5 ml CH₃CN. After 10 min NaBH₃CN (0.0615 g, 0.98 mmol, 1.3 eq) was added. The reaction was adjusted to pH ^{˜}7 with HOAc. After 2 h the solvent was removed in vacuo. The residue was diluted with saturated aqueous NaHCO₃/EtOAc, and the layers were separated. The organic layer was washed with water (×3), brine (×1), and dried over Na₂SO₄. The inorganics were filtered off, and the solvent was removed in vacuo to yield 0.2482 g (0.76 mmol, 100% yield) of tert-butyl 3-(benzyl(methyl)amino)benzylcarbamate.

20% Pd(OH)₂ 0.076 g) was added to a stirred suspension of tert-butyl 3-(benzyl(methyl)amino)benzylcarbamate (0.248 g, 0.76 mmol, 1 eq) in 10 ml EtOH. A H₂ balloon was added. After stirring overnight the mixture was filtered through Celite. The filter cake was rinsed with EtOAc (×3). The organics were combined and the solvent was removed in vacuo to yield tert-butyl 3-(methylamino)benzylcarbamate.

3-(aminomethyl)-N-methylaniline was generated from tert-butyl 3-(methylamino)benzylcarbamate by using a standard deprotection protocol of Boc group described herein.

### Example 1.1.60

### 3-(aminomethyl)-5-methoxy-N,N-dimethylaniline

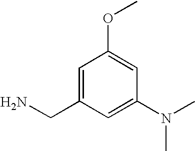

SOCl₂ (3.4 ml, 5.61 g, 47.1 mmol, 5 eq) was added dropwise to a stirred solution of 3,5-dinitrobenzoic acid (2.0 g, 9.43 mmol, 1 eq, Aldrich) in 20 ml anhydrous MeOH at 0° C. under Ar. The reaction was stirred at 0° C. to room temperature overnight. The solvent was removed in vacuo, and the residue was dissolved in EtOAc. The organic layer was washed with saturated aqueous NaHCO₃ (×2), water (×3), brine (×1), and dried over Na₂SO₄. The inorganics were filtered off, and the solvent was removed in vacuo yielding 2.12 g (9.38 mmol, 99% yield) of methyl 3,5-dinitrobenzoate.

methyl 3,5-dinitrobenzoate (1.5 g, 6.63 mmol, 1 eq) in 10 ml anhydrous MeOH under Ar was heated to reflux at 85° C. LiOMe (1.0 M in MeOH, 13.3 ml, 13.3 mmol, 2 eq) was added to the refluxing solution. After 4 h the reaction was cooled to room temperature, and the mixture was adjusted to pH ^{˜}3 with concentrated HCl. The solvent was removed in vacuo and the residue was dissolved in EtOAc. The organic layer was washed with saturated aqueous NaHCO₃ (×2), water (×3), brine (×1), and dried over Na₂SO₄. The inorganics were filtered off, and the solvent was removed in vacuo. Purification via flash chromatography yielded 0.7705 g (3.70 mmol, 56% yield) of methyl 3-methoxy-5-nitrobenzoate.

Lithium aluminum hydride (0.1116 g, 2.94 mmol, 1.5 eq) was added to a stirred solution of methyl 3-methoxy-5-nitrobenzoate (0.408 g, 1.96 mmol, 1 eq) in 10 ml anhydrous Et₂O at 0° C. under Ar. The reaction was stirred at 0° C. to room temperature overnight. Starting material was still present after stirring overnight. Additional LAH (0.1116 g, 2.94 mmol, 1.5 eq) was added. After 2 h the reaction was quenched with water. The reaction was diluted with saturated aqueous NaHCO₃ and extracted with EtOAc (×2). The combined organics were washed with brine (×1) and dried over Na₂SO₄. The inorganics were filtered off, and the solvent was removed in vacuo. Purification via flash chromatography yielded 0.2463 g (1.33 mmol, 68% yield) of (3-methoxy-5-nitrophenyl)methanol.

Diphenylphosphoryl azide (0.316 ml, 0.4 g, 1.46 ml, 1.1 eq) and 1,8-Diazabicyclo(5.4.0)undec-7-ene (0.219 ml, 0.22 g, 1.46 mmol, 1.1 eq) were added to a stirred solution of (3-methoxy-5-nitrophenyl)methanol in 10 ml anhydrous toluene under Ar. After stirring overnight the solvent was removed in vacuo. Purification via flash chromatography yielded 0.278 g (1.34 mmol, 100% yield) of 1-(azidomethyl)-3-methoxy-5-nitrobenzene.

10% Pd/C (0.028 g) was added to a stirred solution of 1-(azidomethyl)-3-methoxy-5-nitrobenzene in 10 ml MeOH. A H₂ balloon was added. After stirring overnight the mixture was filtered through Celite. The filter cake was rinsed with EtOAc (×3). The organics were removed in vacuo. The residue was dissolved water and extracted with Et₂O (×1). The water was removed in vacuo to yield 0.161 g (1.06 mmol, 100% yield) of the crude 3-(aminomethyl)-5-methoxyaniline which was used without purification.

Et₃N (0.3 ml, 0.2 g, 2.12 mmol, 2 eq) and (Boc)₂O (0.24 ml, 0.23 g, 1.06 mmol, 1 eq) were added sequentially to a stirred solution of 3-(aminomethyl)-5-methoxyaniline (0.161 g, 1.06 mmol, 1 eq) in 10 ml anhydrous MeOH at 0° C. under Ar. After stirring at 0° C. to room temperature overnight the solvent was removed in vacuo. The residue was dissolved in EtOAc, washed with water (×3), brine (×1), and dried over Na₂SO₄. The inorganics were filtered off, and the solvent was removed in vacuo. Purification via flash chromatography yielded 0.0975 g (0.39 mmol, 36% yield) of tert-butyl 3-amino-5-methoxybenzylcarbamate with some impurity.

CH₂O (aq, 37%) (0.12 ml, 0.127 g, 1.56 mmol, 4 eq) was added to a stirred solution of tert-butyl 3-amino-5-methoxybenzylcarbamate (0.0975 g, 0.39 mmol, 1 eq) in 5 ml CH₃CN. After 10 min NaBH₃CN (0.056 g, 0.897 mmol, 2.3 eq) was added. The reaction was adjusted to pH ^{˜}7 with HOAc. After stirring overnight the reaction was diluted with Et₂O/water and the layers were separated. The organic layer was washed with saturated aqueous NaHCO₃ (×2), water (×3), brine (×1), and dried over Na₂SO₄. The inorganics were filtered off, and the solvent was removed in vacuo. Purification via flash chromatography yielded 0.0638 g (0.228 mmol, 58% yield) of tert-butyl 3-(dimethylamino)-5-methoxybenzylcarbamate.

3-(aminomethyl)-5-methoxy-N,N-dimethylaniline was generated from tert-butyl 3-(dimethylamino)-5-methoxybenzylcarbamate by using a standard deprotection protocol of Boc group described herein.

### Example 1.1.61

### (3-methoxy-5-nitrophenyl)methanamine

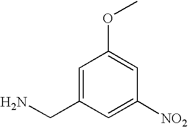

PPh₃ (0.0707 g, 0.27 mmol, 1.1 eq) was added to a stirred solution of 1-(azidomethyl)-3-methoxy-5-nitrobenzene (synthesis described herein) in 5 ml THF. After 5 min 1 ml of water was added, and the reaction was stirred overnight. The solvent was removed in vacuo. The residue was dissolved in EtOAc and extracted with 1N HCl (×1). The aqueous layer was adjusted to pH>8 with 1N NaOH and extracted with EtOAc (×1). This organic fraction was washed with brine (×1) and dried over Na₂SO₄. The inorganics were filtered off, and the solvent was removed in vacuo to yield 0.030 g (0.16 mmol, 67% yield) of the product.

### Example 1.1.62

### 5-(aminomethyl)-N1,N1,N3,N3-tetramethylbenzene-1,3-diamine

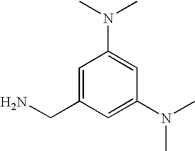

20% Pd(OH)₂ (0.13 g) was added to a stirred suspension of 3,5-dinitrobenzonitrile (0.50 g, 2.59 mmol, 1 eq, Aldrich) in 10 ml EtOH. A H₂ balloon was added. After stirring over the weekend the mixture was filtered through Celite. The filter cake was rinsed with EtOH (×3). The organics were removed in vacuo. The residue was stirred in CHCl₃ and the resulting mixture was filtered (×3). The CHCl₃ fractions were combined, and the solvent was removed in vacuo to yield crude 3,5-diaminobenzonitrile.

CH₂O (aq, 37%) (0.68 ml, 0.7412 g, 9.08 mmol, 6 eq) was added to a stirred solution of 3,5-diaminobenzonitrile (0.2076 g, 1.51 mmol, 1 eq) in 10 ml CH₃CN. After 10 min NaBH₃CN (0.45 g, 6.8 mmol, 4.5 eq) was added. The reaction was adjusted to pH ^{˜}7 with HOAc. After stirring overnight the solvent was removed in vacuo. The residue was dissolved in EtOAc, washed with saturated aqueous NaHCO₃ (×2), water (×3), brine (×1), and dried over Na₂SO₄. The inorganics were filtered off, and the solvent was removed in vacuo. Purification via flash chromatography yielded 0.1145 g (0.59 mmol, 39% yield) of 3,5-bis(dimethylamino)benzonitrile.

NaBH₄ (0.067 g, 1.77 mmol, 3 eq) was added to a stirred solution of 3,5-bis(dimethylamino)benzonitrile (0.1145 g, 0.59 mmol, 1 eq) and CoCl₂.6H₂O (0.0161 g, 0.059 mmol, 10 mol %) in 5 ml EtOH at 50° C. After 2 h the reaction was not complete. Additional NaBH₄ (0.0245 g, 0.64 mmol, 1.1 eq) was added. After 2 h the reaction was cooled to room temperature and 3 N HCl was added to pH=1-2. After stirring for 1 h the reaction was concentrated in vacuo. The residue was extracted with EtOAc (×2). The aqueous layer was adjusted to pH=8-9 with 1 N NaOH. The water was removed in vacuo. The residue was stirred with CHCl₃, filtered through Celite, and dried over Na₂SO₄. The inorganics were filtered off, and the solvent was removed in vacuo yielding 0.0807 g (0.22 mmol, 37% yield) of the product.

### Example 1.1.63

### 4-(aminomethyl)-6-methoxypyrimidin-2-amine

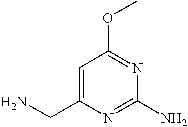

NaH (60% dispersion in oil, 0.201 g, 5.02 mmol, 2 eq) was added to a stirred solution of 4-chloro-6-methoxypyrimidin-2-amine (0.400 g, 2.51 mmol, 1 eq, Aldrich) and MeI (0.5 ml, 1.07 g, 7.52 mmol, 3 eq) in 2 ml anhydrous DMF at 0° C. After 1 h the reaction was quenched with water and diluted with EtOAc. The layers were separated. The organic layer was washed with water (×3), brine (×1), and dried over Na₂SO₄. The inorganics were filtered off, and the solvent was removed in vacuo. Purification via flash chromatography yielded 0.180 g (0.96 mmol, 38% yield) of 4-chloro-6-methoxy-N,N-dimethylpyrimidin-2-amine.

A solution of 4-chloro-6-methoxy-N,N-dimethylpyrimidin-2-amine (0.350 g, 2.19 mmol, 1 eq) in 10 ml anhydrous DMF was degassed with Ar for 5 min. Zn(CN)₂ (0.155 g, 1.32 mmol, 0.6 eq) and Pd(PPh₃)₄ (0.253 g, 0.219 mmol, 10 mol %) were added and the mixture was heated to 95° C. After 23 h the reaction was cooled to room temperature and the mixture was diluted with Et₂O and NH₄OH (28%). After stirring for 1 h the layers were separated. The organic layer was washed with water (×3), brine (×1), and dried over Na₂SO₄. The inorganics were filtered off, and the solvent was removed in vacuo. Purification via flash chromatography yielded 0.214 g (1.43 mmol, 65% yield) of 2-amino-6-methoxypyrimidine-4-carbonitrile.

10% Pd/C (0.010 g) was added to a stirred solution of 2-amino-6-methoxypyrimidine-4-carbonitrile (0.107 g, 0.71 mmol, 1 eq) in 4 ml HOAc. A H₂ balloon (20 psi) was added. After 2 h the mixture was filtered through Celite. The filter cake was rinsed with MeOH. The organics were combined and the solvent was removed in vacuo. 1 N NaOH was added to pH>8 and the solvent was removed in vacuo. The residue was stirred in EtOAc (30 ml) and saturated aqueous NaHCO₃ (1 ml). After 30 min Na₂SO₄ was added and the mixture was stirred for 10 min. The inorganics were filtered off, and the solvent was removed in vacuo yielding 0.1044 g (0.68 mmol, 95% yield) of the product.

### Example 1.1.64

### 1-(5-isopropylpyridin-3-yl)ethanamine

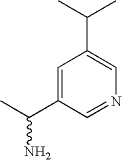

A stirred solution of 1-(5-bromopyridin-3-yl)ethanone (600 mg, 3.0 mmol), potassium isopropenyltrifluoroborate (148 mg, 3.0 mmol), and Et₃N (1.25 mL, 9.0 mmol) in ⁱPrOH (20 mL) and H₂O (10 mL) was degassed with argon for 10 min and then PdC12 (dppf).CH₂Cl₂ (74 mg, 0.09 mmol) was added and the reaction mixture was heated to reflux for 5 h. The solution was cooled to room temperature and diluted with Ether and H₂O. The layers were separated and the aqueous layer was extracted with Ether (2×50 mL). The combined organic layer was washed with brine, dried with Na₂SO₄ and concentrated under reduced pressure. The residue was purified by column chromatography (30% EtOAc in hexanes) to provide 460 mg of 1-(5-(prop-1-en-2-yl)pyridin-3-yl)ethanone.

To a stirred solution of 1-(5-(prop-1-en-2-yl)pyridin-3-yl)ethanone (460 mg, 2.86 mmol) in 95% EtOH was added a solution of NH₂OH.HCl (1.19 g, 17.14 mmol) in a mixture of water (5.8 mL) and 10% NaOH (5.8 mL) and refluxed for 2.5 h. All solvent was removed. The residue was dissolved in CHCl₃, washed with water, dried with Na₂SO₄ and concentrated under reduced pressure to yield 400 mg of crude 1-(5-(prop-1-en-2-yl)pyridin-3-yl)ethanone oxime.

To a stirred solution of 1-(5-(prop-1-en-2-yl)pyridin-3-yl)ethanone oxime (400 mg) in MeOH.HCl (1.25M, 8 mL) was added 10% Pd/C (40 mg) and stirred under hydrogen atmosphere for 4 h. The catalyst was filtered off and the residue was dissolved in 5 mL of 25% aqueous NH₃ and extracted with CHCl₃ and the organic layer was washed with brine, dried with Na₂SO₄ and concentrated under reduced pressure. The residue was purified by column chromatography (10% MeOH/90% CHCl₃ spiked with 0.25% of Et₃N) to provide 155 mg of the product.

### Example 1.1.65

### (R)-1-(5-(prop-1-en-2-yl)pyridin-3-yl)ethanamine

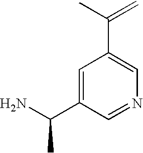

To a stirred solution of 1-(5-bromopyridin-3-yl)ethanone (5.0 g, 25.0 mmol) in anhydrous MeOH (50 mL) at 0° C., was added NaBH₄ (1.32 g, 35.0 mmol) and the reaction was allowed to warm to room temperature. All solvent was removed. The residue was dissolved in cold water and extracted with EtOAc (2×70 mL), The combined organic layers was dried with Na₂SO₄ and concentrated under reduced pressure. The residue was purified by column chromatography (40% EtOAc in hexanes) to provide 4.555 g of 1-(5-bromopyridin-3-yl)ethanol.

To a stirred solution of 1-(5-bromopyridin-3-yl)ethanol (2.0 g) in anhydrous Et₂O (50 mL) at 0° C., was vinyl acetate (2 mL), 4A° molecular sieves (2.0 g), Lipase immobilized from Candida Antarctica(200 mg) and the reaction was stirred for 16 h. The catalyst and molecular sieves were filtered off and the solvent was concentrated under reduced pressure. The residue was purified by column chromatography (30% EtOAc in hexanes) to provide 1.0 g of (S)-1-(5-bromopyridin-3-yl)ethanol and 1.18 g of (R)-1-(5-bromopyridin-3-yl)ethyl acetate.

A stirred solution of (S)-1-(5-bromopyridin-3-yl)ethanol (950 mg, 4.7 mmol), potassium isopropenyltrifluoroborate (730 mg, 4.94 mmol), and Et₃N (1.95 mL, 14.1 mmol) in ⁱPrOH (30 mL) and H₂O (15 mL) was degassed with argon for 10 min and then PdC12 (dppf).CH₂Cl₂ (192 mg, 0.24 mmol) was added and the reaction mixture was heated to reflux for 4 h. The solution was cooled to room temperature and diluted with Ether and H₂O. The layers were separated and the aqueous layer was extracted with Ether (2×50 mL). The combined organic layer was washed with brine, dried with Na₂SO₄ and concentrated under reduced pressure. The residue was purified by column chromatography (60% EtOAc in hexanes) to provide 520 mg of (S)-1-(5-(prop-1-en-2-yl)pyridin-3-yl)ethanol.

DPPA (0.55 mL, 2.55 mmol) was added to (S)-1-(5-(prop-1-en-2-yl)pyridin-3-yl)ethanol (347 mg, 2.13 mmol) in toluene (5 mL) and the reaction is cooled to 0° C. DBU (0.38 mL, 2.55 mmol) was added, the reaction was allowed to warm to room temperature and stirred overnight, diluted with EtOAc and washed with water. The organic layer was dried with Na₂SO₄ and concentrated under reduced pressure. The residue was purified by column chromatography (35% EtOAc in hexanes) to provide 340 mg of (R)-3-(1-azidoethyl)-5-(prop-1-en-2-yl)pyridine.

To a stirred solution of (R)-3-(1-azidoethyl)-5-(prop-1-en-2-yl)pyridine (340 mg) in MeOH.HCl (1.25M, 10 mL) was added 10% Pd/C (50 mg) and stirred under hydrogen atmosphere for 12 h. The catalyst was filtered off and the residue was dissolved in 5 mL of 25% aqueous NH₃ and extracted with CHCl₃ and the organic layer was washed with brine, dried with Na₂SO₄ and concentrated under reduced pressure. The residue was purified by column chromatography (10% MeOH/90% CHCl₃ spiked with 0.25% of Et₃N) to provide 246 mg of (R)-1-(5-(prop-1-en-2-yl)pyridin-3-yl)ethanamine.

### Example 1.1.66

### (S)-1-(5-(prop-1-en-2-yl)pyridin-3-yl)ethanamine

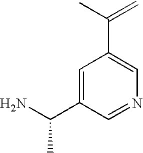

To a stirred solution of (R)-1-(5-bromopyridin-3-yl)ethyl acetate (1.21 g, 4.96 mmol, synthesis described herein) in MeOH (20 mL), was added K₂CO₃ (1.37 g, 9.92 mmol) and the reaction was stirred for 1 h. All the solvent was removed, the residue was dissolved in cold water and extracted with EtOAc (2×50 mL), to provide 1.0 g of crude (R)-1-(5-bromopyridin-3-yl)ethanol.

(R)-1-(5-bromopyridin-3-yl)ethanol was transformed into (S)-1-(5-(prop-1-en-2-yl)pyridin-3-yl)ethanamine following same chemistry as described for (R)-1-(5-(prop-1-en-2-yl)pyridin-3-yl)ethanamine.

### Example 1.1.67

### (3-isopropyl-5-(methylsulfonyl)phenyl)methanamine

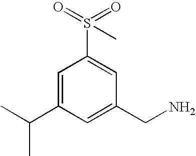

To a stirring slurry of 3.2 g (25.4 mmol) of Na₂SO₃ and 6.2 g (73.8 mmol) of NaHCO₃ in 20 mL of H₂O at 75° C. was added 5.0 g (24.4 mmol) of 3,5-dimethylbenzene-1-sulfonyl chloride in several portions. After 2 h at 75° C., 3.5 g (37.0 mmol) of chloroacetic acid was added in portions followed by 1.5 g (37.5 mmol) of NaOH in 3 mL of H₂O. The mixture was stirred at 135° C. for 13 h, and 3 N HCl was added to a pH=1. A colorless precipitate formed, and the mixture was filtered through a Buchner funnel to provide 3.8 g of 1,3-dimethyl-5-(methylsulfonyl)benzene in 84% yield.

To a stirring mixture of 5.2 g (28.2 mmol) of 1,3-dimethyl-5-(methylsulfonyl)benzene in 25 mL of pyridine and 50 mL of H₂O was added 27 g of KMnO₄ in 9, 3 g portions at 120° C. After the mixture was stirred at 120° C. overnight, the mixture was filtered hot through a Buchner funnel. The solution was washed with CHCl₃ about 2-3 times and acidified to a pH=1-2. The colorless precipitate of 5-(methylsulfonyl)isophthalic acid that formed was filtered through a Buchner funnel and used in the next reaction without further purification.

To a stirring mixture of 3.5 g (14.3 mmol) of 5-(methylsulfonyl)isophthalic acid in 60 mL of MeOH was added 11.5 mL (158 mmol) of SOCl₂ dropwise over a period of about 30 minutes. After 39 h, the solution was concentrated and sat. NaHCO₃ and CHCl₃ were added. The extract was dried over Na₂SO₄, filtered, and concentrated. The dimethyl 5-(methylsulfonyl)isophthalate product was used in the next reaction without further purification.

To a stirring solution of 4.57 g (16.8 mmol) of dimethyl 5-(methylsulfonyl)isophthalate in 100 mL of THF and 100 mL of MeOH was added 671 mg (16.8 mmol) of NaOH in 17 mL of water. The solution was allowed to stir at r.t. for 29.5 h and then concentrated. The aqueous layer was washed with CHCl₃ (2×) and then acidified to pH=1 with 1 N HCl. A precipitate formed, and the mixture was filtered through a Buchner funnel. The colorless solid of 3-(methoxycarbonyl)-5-(methylsulfonyl)benzoic acid (2.85 g-62% yield) was used without further purification.

The corresponding azide, methyl 3-(azidomethyl)-5-(methylsulfonyl)benzoate, was synthesized from 3-(methoxycarbonyl)-5-(methylsulfonyl)benzoic acid following the general procedure as described herein.

A mixture of 147 mg (0.546 mmol) of methyl 3-(azidomethyl)-5-(methylsulfonyl)benzoate and 18.5 mg of 10% Pd/C in 6 mL of MeOH was stirred at r.t. under H₂ balloon for 3 h. The mixture was filtered through Celite and concentrated. The crude (3-isopropyl-5-(methylsulfonyl)phenyl)methanamine (101 mg) was used in the next reaction without further purification.

### Example 1.1.68

### (5-isopropyl-2-methylpyridin-3-yl)methanamine

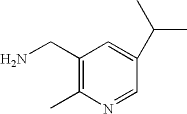

To a stirring solution of 1.1 g (9.47 mmol) of methyl acetoacetate in 20 mL of THF at 0° C. was added about 10 mL of ^{t}BuOK (1.0 M) in THF dropwise. The ice bath was removed and stirring was continued at r.t. After 1 h 0.5 mL of ^{t}BuOK was added, and after 10 min. 1.12 g (10 mmol) of DABCO (1,4-Diazabicyclo[2.2.2] octane) and 4.23 g (13.8 mmol) of the vinamidinium hexafluorophosphate salt (Davies, I. W., et al. J. Org. Chem. 2000, 65, 4571) were added. The mixture was heated at 45° C. and after 3 h, 1.35 g (17.5 mmol) of NH₄OAc was added. The temperature was increased to 80° C., and after 1 hour, 130 mg of NH₄OAc was added. After 4 h, the reaction was quenched with 45 mL of water. The aqueous layer was extracted with EtOAc (4×), and the combined extracts were washed with brine, dried over Na₂SO₄, filtered, and concentrated. Purification by flash silica gel chromatography (45% EtOAc/hexanes) provided 1.44 g of methyl 5-chloro-2-methylnicotinate as a yellow orange oil in about 80% yield.

To a stirring solution of 950 mg (5.12 mmol) of methyl 5-chloro-2-methylnicotinate in 20 mL of THF at 0° C. was added 500 mg (13.2 mmol) of LiAlH₄ in 2 portions. After stirring at 0° C. for about 1 h., the following were added in succession: 0.5 mL of H₂O, 0.5 mL of 15% NaOH (aqueous), and 1.5 mL of brine. The ice bath was removed and stirring was continued for 2 h. The mixture was filtered through Celite and concentrated. Purification by flash silica gel chromatography (2% MeOH/CHCl₃) provided 399 mg of (5-chloro-2-methylpyridin-3-yl)methanol as an orange-yellow oil in 50% yield.

To a stirring solution of 399 mg (2.55 mmol) of (5-chloro-2-methylpyridin-3-yl)methanol in 10 mL of toluene at r.t. was added 660 μL (3.06 mmol) of DPPA (diphenylphosphoryl azide). The solution was cooled to 0° C. and 450 μL of DBU (1,8-diazabicyclo[5.4.0] undec-7-ene) was added. The ice bath was removed and stirring was continued with warming to r.t. After 16 h, 1 N HCl was added to a pH=8. Water was added and the aqueous layer was extracted with EtOAc. The organic layer was washed with brine, dried over Na₂SO₄, filtered, and concentrated. Purification by flash silica gel chromatography (45% EtOAc/hexanes) provided 3-(azidomethyl)-5-chloro-2-methylpyridine as a yellow oil in quantitative yield.

(5-chloro-2-methylpyridin-3-yl)methanamine was synthesized from 3-(azidomethyl)-5-chloro-2-methylpyridine following the general procedure as described herein.

The Boc protected amine (tert-butyl (5-chloro-2-methylpyridin-3-yl)methylcarbamate) was synthesized from (5-chloro-2-methylpyridin-3-yl)methanamine following the general procedures as described herein.

To a solution of 262 mg (1.02 mmol) of tert-butyl (5-chloro-2-methylpyridin-3-yl)methylcarbamate, 163 mg (1.08 mmol) of potassium isopropenyltrifluoroborate, and 450 μL (4.28 mmol) of ^{t}BuNH₂ in 7.2 mL of isopropanol and 2.8 mL of H₂O (degassed) was added 85.5 mg (0.105 mmol) of PdCl₂(dppf).CH₂Cl₂. After heating the solution for 15 h at 120° C., H₂O and EtOAc were added. The organic layer was washed with brine, dried over Na₂SO₄, filtered, and concentrated. Purification by flash silica gel chromatography (40% EtOAc/hexanes) provided 31.7 mg of 1-(2-methyl-5-(prop-1-en-2-yl)pyridin-3-yl)-N-((oxoboryl)methylene)methanamine as a yellow oil in 12% yield.

To a stirring solution of 96.6 mg (0.370 mmol) of 1-(2-methyl-5-(prop-1-en-2-yl)pyridin-3-yl)-N-((oxoboryl)methylene)methanamine and 88.5 mg (0.372 mmol) of CoCl₂.6H₂O in 4 mL of EtOH at 50° C. was added 160 mg of NaBH₄ in 2 portions. After heating the mixture at 50° C. under Ar for 5 h, the reaction mixture was cooled and 5 N HCl was added to a pH=1. The mixture was stirred at r.t. for 15 h and concentrated. Water was added, and NH₄OH was added to a pH=9. The aqueous layer was extracted with the extract of (40 mL of CHCl₃: 5 mL of MeOH: 5 mL H₂O) (2×). The combined extracts were dried over Na₂SO₄, filtered, and concentrated. (5-isopropyl-2-methylpyridin-3-yl)methanamine was used in the next reaction without further purification.

### Example 1.1.69

### (3-(benzyloxy)-5-(2-chloropropan-2-yl)phenyl)methanamine

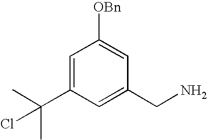

A mixture of 1.98 g (9.42 mmol) of dimethyl 5-hydroxyisophthalate 3.0 g (21.7 mmol) of K₂CO₃ and 1.8 mL (15.1 mmol) of BnBr in 30 mL of DMF was heated at 60° C. for 18 h. After the mixture was filtered through cotton, water and CHCl₃ were added. The organic layer was washed with water and brine, dried over Na₂SO₄, filtered, and concentrated. Purification by flash silica gel chromatography (15% EtOAc/hexanes) provided the 2.64 g of dimethyl 5-(benzyloxy)isophthalate as a colorless solid in 93% yield.

3-(benzyloxy)-5-(methoxycarbonyl)benzoic acid was synthesized from the dimethyl 5-(benzyloxy)isophthalate following the general procedures as described herein.

1-(azidomethyl)-3-(benzyloxy)-5-(2-chloropropan-2-yl)benzene was synthesized from 3-(benzyloxy)-5-(methoxycarbonyl)benzoic acid following the general procedures as described herein.

A mixture of 103 mg of 1-(azidomethyl)-3-(benzyloxy)-5-(2-chloropropan-2-yl)benzene and 10.4 mg of 10% Pd/C in 5 mL of MeOH was stirred at r.t. under H₂ balloon for 4.5 h. The mixture was filtered through Celite and concentrated. (3-(benzyloxy)-5-(2-chloropropan-2-yl)phenyl)methanamine was used in the next reaction without further purification.

### Example 1.1.70

### 3-(aminomethyl)-5-isopropylphenyl methanesulfonate

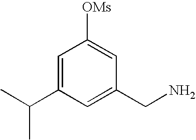

1-(azidomethyl)-3-(benzyloxy)-5-(prop-1-en-2-yl)benzene was synthesized from 3-(benzyloxy)-5-(methoxycarbonyl)benzoic acid following the general procedures as described herein.

A mixture of 573 mg (1.97 mmol) of 1-(azidomethyl)-3-(benzyloxy)-5-(prop-1-en-2-yl)benzene, 0.5 mL of BOC₂O, and 60.5 mg of 10% Pd/C in 10 mL of EtOAc was stirred at r.t. under H₂ balloon for 11.5 h. The mixture was filtered through Celite and concentrated. Purification by flash silica gel chromatography (10% EtOAc/hexanes) provided 336 mg of tert-butyl 3-(benzyloxy)-5-isopropylbenzylcarbamate in 62% yield.

tert-butyl 3-hydroxy-5-isopropylbenzylcarbamate was synthesized from tert-butyl 3-(benzyloxy)-5-isopropylbenzylcarbamate following the general procedures as described herein.

To a stirring solution of 91.8 mg of tert-butyl 3-hydroxy-5-isopropylbenzylcarbamate in 3 mL of CH₂Cl₂ and 300 μL of pyridine at 0° C. was added 30 μL (0.386 mmol) of MsCl. The ice bath was removed, and after stirring at r.t. for 75 min. 50 μL of Et₃N was added. After 30 min., the solution was concentrated and EtOAc and H₂O were added. The organic layer was washed with 15 mL of brine, and the aqueous layer was extracted with EtOAc. The combined extracts were dried over Na₂SO₄, filtered, and concentrated. Purification by flash silica gel chromatography (40% EtOAc/hexanes) provided 96.2 mg of 3-((tert-butoxycarbonylamino)methyl)-5-isopropylphenyl methanesulfonate as a pale yellow oil in about 80% yield.

3-(aminomethyl)-5-isopropylphenyl methanesulfonate was generated from 3-((tert-butoxycarbonylamino)methyl)-5-isopropylphenyl methanesulfonate by using a standard deprotection protocol of Boc group described herein.

### Example 1.1.71

### (3-(ethylsulfonyl)-5-isopropylphenyl)methanamine

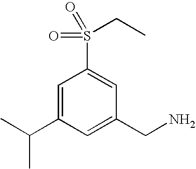

To a stirring solution of 4.1 g (73.1 mmol) of KOH in 70 mL of MeOH at r.t. was added 5 g (36.2 mmol) of 3,5-dimethylbenzenethiol. After 1.5 h, 5.4 mL of EtBr was added, and the solution was stirred at r.t. for about 40 min. and heated at 60° C. for 75 min. Water (300 mL) and CH₂Cl₂ (100 mL) were added, and the aqueous layer was extracted with CH₂Cl₂ (2×) (1×100 mL and 1×50 mL). The combined extracts were dried over Na₂SO₄, filtered, and concentrated. The crude yellow liquid of (3,5-dimethylphenyl)(ethyl)sulfane was used in the next reaction without further purification.

To stirring mixture of (3,5-dimethylphenyl)(ethyl)sulfane in 15 mL of pyridine and 50 mL of H₂O at 120° C. was added 36 g of KMnO₄ by 3 g portions. After the mixture was heated at 120° C. for 12.5 h, it was allowed to cool and was filtered through a Buchner funnel. The aqueous layer was extracted with CHCl₃ (2×), and concentrated HCl was added to the aqueous layer to a pH=1. A solid precipitated, and the mixture was filtered through a Buchner funnel to give 1.86 g (31% yield) of 5-(ethylsulfonyl)isophthalic acid as a colorless solid which was used without further purification.

(3-(ethylsulfonyl)-5-isopropylphenyl)methanamine was synthesized from 5-(ethylsulfonyl)isophthalic acid using a standard deprotection protocol of Boc group described herein.

### Example 1.1.72

### N-(3-(aminomethyl)-5-isopropylphenyl)acetamide

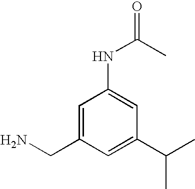

To a stirring mixture of 2.4 g (9.92 mmol) of 2-amino-3-bromo-5-nitrobenzonitrile in 56 mL of EtOH and 5.6 mL of H₂SO₄ at 90° C. was added 5.0 g of NaNO₂ in several portions. After 36.5 h, H₂O was added, and the aqueous layer was extracted with CHCl₃. More H₂O was added and the aqueous layer was extracted with CHCl₃. The combined extracts were dried over Na₂SO₄, filtered, and concentrated. Purification by flash silica gel chromatography (5% EtOAc/hexanes) resulted in 3-bromo-5-nitrobenzonitrile as a yellow solid.

To a stirring solution of 845 mg (3.72 mmol) of 3-bromo-5-nitrobenzonitrile in 5 mL of THF and 5 mL of EtOH was added 4.2 g (18.6 mmol) of SnCl₂.2H₂O in several portions. The reaction became slightly exothermic and was stirred at r.t. for about 12.5 h. The mixture was concentrated and 30 mL of 2 N NaOH was added. After the mixture was stirred for 2 h, H₂O and EtOAc were added, and the organic layer was washed with 50 mL of H₂O and 40 mL of brine. The aqueous layer was extracted with EtOAc and washed with brine. The combined extracts were dried over Na₂SO₄, filtered, and concentrated. Purification by flash silica gel chromatography (35% EtOAc/hexanes) provided 461 mg of 3-amino-5-bromobenzonitrile as a yellow solid in 63% yield.

To a stirring solution of 103 mg (0.520 mmol) of 3-amino-5-bromobenzonitrile in 3 mL of pyridine was added 0.12 mL (1.27 mmol) of Ac₂O. The solution was stirred at r.t. for about 5.5 h and concentrated. Ethyl acetate was added, and the organic layer was washed with H₂O and brine, dried over Na₂SO₄, filtered and concentrated. N-(3-bromo-5-cyanophenyl)acetamide was used for the next reaction without further purification.

N-(3-cyano-5-(prop-1-en-2-yl)phenyl)acetamide was synthesized from N-(3-bromo-5-cyanophenyl)acetamide following the general procedure as described herein.

To a stirring solution of 91.9 mg (0.459 mmol) of N-(3-cyano-5-(prop-1-en-2-yl)phenyl)acetamide and 110 mg (0.466 mmol) of CoCl₂ 6H₂O in 3 mL of EtOH at 50° C. was added 118 mg of NaBH₄ in 2 portions. After 3 h 45 min., 5 N HCl was added to a pH=1, and the mixture was concentrated. Ammonium hydroxide was added to a pH=9, and H₂O was also added. The aqueous layer was extracted with the extract of (40 mL CHCl₃: 5 mL of MeOH: 5 mL of H₂O) (2×). The combined extracts were dried over Na₂SO₄, filtered, and concentrated to provide the crude N-(3-(aminomethyl)-5-isopropylphenyl)acetamide which was used without further purification.

### Example 1.1.73

### 3-(aminomethyl)-5-isopropylphenyl dimethylcarbamate

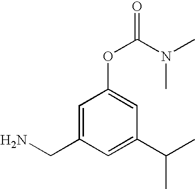

A solution of 57.4 mg (0.216 mmol) of tert-butyl 3-hydroxy-5-isopropylbenzylcarbamate (synthesis described herein) and 30 pt (0.327 mmol) of dimethylcarbamyl chloride in 3 mL of pyridine was heated at 120° C. for 16 h. The solution was concentrated, and CHCl₃ and H₂O were added. The organic layer was washed with brine (10 mL), dried over Na₂SO₄, filtered and concentrated. Purification by flash silica gel chromatography (25% EtOAc/hexanes) provided 46 mg of Boc-protected 3-(aminomethyl)-5-isopropylphenyl dimethylcarbamate as a yellow oil in 63% yield.

A solution of 46 mg (0.137 mmol) of the Boc-protected 3-(aminomethyl)-5-isopropylphenyl dimethylcarbamate in 2.5 mL of 1.25 M HCl in MeOH was stirred at r.t. for 3.5 h. Trifluoroacetic acid (0.2 mL) was added and stirring was continued for 20 min., and the solution was concentrated. Saturated NaHCO₃ was added, and the aqueous layer was extracted with the extract of (40 mL CHCl₃: 5 mL of MeOH: 5 mL of H₂O) (3×). The combined extracts were dried over Na₂SO₄, filtered, and concentrated. Crude 3-(aminomethyl)-5-isopropylphenyl dimethylcarbamate was used in the next reaction without further purification.

### Example 1.1.74

### methyl 3-(aminomethyl)-5-isopropylphenylcarbamate

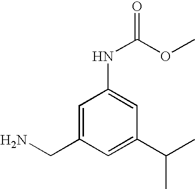

To a stirring solution of 97.3 mg (0.494 mmol) of 3-amino-5-bromobenzonitrile in 3 mL of pyridine at r.t. was added 0.1 mL of methyl chloroformate. After 15 h, 15 mL of H₂O and EtOAc were added. The organic layer was washed with H₂O and brine, dried over Na₂SO₄, filtered, and concentrated. Crude methyl 3-bromo-5-cyanophenylcarbamate was used in the next reaction without further purification.

methyl 3-cyano-5-(prop-1-en-2-yl)phenylcarbamate was synthesized from methyl 3-bromo-5-cyanophenylcarbamate following the general procedures as described herein.

methyl 3-(aminomethyl)-5-(prop-1-en-2-yl)phenylcarbamate was synthesized from methyl 3-cyano-5-(prop-1-en-2-yl)phenylcarbamate following the general procedures as described herein.

A mixture of about 92 mg of crude methyl 3-(aminomethyl)-5-(prop-1-en-2-yl)phenylcarbamate and 17.6 mg of 10% Pd/C in 7 mL of MeOH and 2 mL of EtOAc was stirred at r.t. under H₂ balloon for 16 h. The mixture was filtered through Celite and concentrated to give the amine product which was used without further purification.

### Example 1.1.75

### 3-(aminomethyl)-5-tert-butylphenol

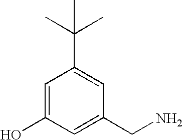

To a stirring solution of 5.0 g (33.5 mmol) of 4-tert-butylaniline in 250 mL of CH₂Cl₂ at 0° C. was added 6 mL of Br₂ in 30 mL of CH₂Cl₂ dropwise until a dark orange color persisted. The organic layer was washed with 100 mL of water, 100 mL of sat. NaHCO₃, and 100 mL of brine. It was dried over Na₂SO₄, filtered, and concentrated to form 2,6-dibromo-4-tert-butylaniline which was used in the next reaction without further purification.

To a stirring solution of 2,6-dibromo-4-tert-butylaniline in 115 mL of EtOH was added 11.5 mL of concentrated H₂SO₄. To the stirring solution at 90° C. was added 8.8 g of NaNO₂ in several portions. After 37 h, EtOAc and 120 mL of H₂O were added, and the layers separated. The organic layer was washed with 40 mL of brine, dried over Na₂SO₄, filtered, and concentrated. Purification by flash silica gel chromatography (hexanes) provided 8.15 g of 1,3-dibromo-5-tert-butylbenzene as a yellow-brown oil with some impurity.

To a stirring solution of 34 mL of 1.6 M n-BuLi in hexanes in 20 mL of THF at −78° C. was added 8.15 g of 1,3-dibromo-5-tert-butylbenzene in 80 mL of THF dropwise over a period of 1 h. After 1 h at −78° C., 4.6 mL of B(OMe)₃ was added, and after 20 min. the cold bath was removed and stirring continued with warming to r.t. After 1 h, EtOAc and 70 mL of 1 N HCl were added, and the layers were separated. The organic layer was washed with brine, dried over Na₂SO₄, filtered, and concentrated. To a stirring mixture of the crude product in 105 mL of 1 N NaOH at 0° C. was added 22 mL of H₂O₂ (30 wt % in H₂O) dropwise. After 25 min. 5 N HCl was added to a pH=1. Ethyl acetate was added, and the organic layer was washed with 80 mL of brine, dried over Na₂SO₄, filtered, and concentrated. Purification by flash silica gel chromatography (hexanes to 10% EtOAc/hexanes) provided 6.54 g of 3-bromo-5-tert-butylphenol as an orange oil with some impurity.

A solution of 403 mg of 3-bromo-5-tert-butylphenol and 200 mg (1.28 mmol) of CuCN in 5 mL of DMF was stirred at 160° C. After 75 min. 365 mg of CuCN was added and stirring was continued at 160° C. After about 1 h, the temperature was increased to 170° C. and the mixture was refluxed for 19 h. Ethyl acetate and H₂O were added, and the mixture was filtered through a Buchner funnel, and the layers were separated. The organic layer was washed with 10 mL of H₂O and brine, dried over Na₂SO₄, filtered, and concentrated. Purification by flash silica gel chromatography (15% EtOAc/hexanes) provided 153 mg of 3-tert-butyl-5-hydroxybenzonitrile as an orange solid in 49% yield.

3-(aminomethyl)-5-tert-butylphenol was synthesized from 3-tert-butyl-5-hydroxybenzonitrile following the general procedures as described herein.

### Example 1.1.76

### (3-isopropyl-5-(methylsulfonylmethyl)phenyl)methanamine

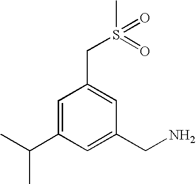

To stirring solution of 5.0 g (18.9 mmol) of 3,5-dibromobenzaldehyde in 30 mL of MeOH and 25 mL of THF at 0° C. was added 819 mg of NaBH₄ in 3 portions. The yellow solution was stirred at 0° C. for 30 min. and then concentrated. Water and EtOAc were added, and 1 N HCl was added to a pH=7. The organic layer was washed with 25 mL of brine, dried over Na₂SO₄, filtered, and concentrated. (3,5-dibromophenyl)methanol was used in the next reaction without further purification.

To a stirring cloudy solution of 2.41 g (9.04 mmol) of (3,5-dibromophenyl)methanol in 40 mL of CH₂Cl₂ at r.t. was added 1.4 mL of Et₃N and 62.7 mg of DMAP. The solution was cooled to 0° C. and 1.0 mL of MsCl was added, and the solution was gradually allowed to warm to r.t. After 24.5 h, the organic layer was washed with water (20 mL) and brine (15 mL), dried over Na₂SO₄, filtered, and concentrated. Purification by flash silica gel chromatography (5% EtOAc/hexanes) provided 1.88 g of 1,3-dibromo-5-(chloromethyl)benzene as a yellow oil in 73% yield and 476 mg of the mesylate (35% EtOAc/hexanes) as a pale yellow solid.

A mixture of 1.88 g (6.61 mmol) of 1,3-dibromo-5-(chloromethyl)benzene and 456 mg (6.50 mmol) of NaSMe in 13 mL of EtOH was stirred at 95° C. After 4 h, 97.1 mg of NaSMe was added and after 30 min. EtOAc and H₂O were added. The layers were separated, and the organic layer was washed with 30 mL of brine, dried over Na₂SO₄, filtered, and concentrated. (3,5-dibromobenzyl)(methyl)sulfane was used in the next reaction without further purification.

To a stirring solution of the crude (3,5-dibromobenzyl)(methyl)sulfane in 15 mL of MeOH at 0° C. was added 12.2 g of Oxone in 15-20 mL of H₂O. The slurry was stirred at 0° C. for 1.5 h and then H₂O and EtOAc were added. The organic layer was washed with 20 mL of brine, dried over Na₂SO₄, filtered, and concentrated. 1,3-dibromo-5-(methylsulfonylmethyl)benzene was used in the next reaction without further purification.

A mixture of 1.37 g (4.17 mmol) of 1,3-dibromo-5-(methylsulfonylmethyl)benzene, 297 mg (2.53 mmol) of Zn(CN)₂, and 298 mg (0.258 mmol) of Pd(PPh₃)₄ in 10 mL of DMF (degassed) was heated at 80° C. After 2.5 h, a 10% NH₄OH aqueous solution was added, and the aqueous layer was extracted with EtOAc. The organic layer was washed with H₂O and brine, dried over Na₂SO₄, filtered, and concentrated. Purification by flash silica gel chromatography (50-60) % EtOAc/hexanes provided 228 mg of 3-bromo-5-(methylsulfonylmethyl)benzonitrile as a colorless solid in 20% yield.

3-(methylsulfonylmethyl)-5-(prop-1-en-2-yl)benzonitrile was synthesized from 3-bromo-5-(methylsulfonylmethyl)benzonitrile following the general procedures as described herein.

(3-isopropyl-5-(methylsulfonylmethyl)phenyl)methanamine was synthesized from 3-(methylsulfonylmethyl)-5-(prop-1-en-2-yl)benzonitrile following the general procedures as described herein.

### Example 1.1.77

### (3-isopropyl-5-(isopropylsulfonyl)phenyl)methanamine

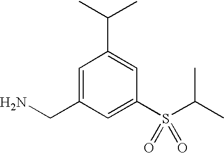

To a degassed mixture of 5.0 g (17.9 mmol) of 3,5-dibromophenylboronic acid in 50 mL of THF and 25 mL of 2 M Na₂CO₃ (aqueous) was added 1.13 g (0.975 mmol) of Pd(PPh₃)₄ and 2.3 mL of 2-bromopropene. The mixture was stirred at 40° C. for 5 h, and the mixture was extracted with EtOAc. The organic layer was washed with 25 mL of brine, dried over Na₂SO₄, filtered, and concentrated. Purification by flash silica gel chromatography (10% EtOAc/hexanes) provided 1,3-dibromo-5-(prop-1-en-2-yl)benzene with some impurity.

To a stirring mixture of 20 mL of ^{t}BuLi (1.7 M in pentane) in 15 mL of THF at −78° C. was added 3.1 g of 1,3-dibromo-5-(prop-1-en-2-yl)benzene in 50 mL of THF dropwise over a period of 20 min. After 55 min. 804 mg (25.1 mmol) of sulphur was added. The cooling bath was removed and stirring was continued with warming to r.t., and after 2.5 h, 1.6 mL of isopropyl iodide was added. After the stirring with the light off for 23.5 h, EtOAc and water were added. The organic layer was washed with brine, dried over Na₂SO₄, filtered, and concentrated. Purification by flash silica gel chromatography (hexanes) provided 382 mg of (3-bromo-5-(prop-1-en-2-yl)phenyl)(isopropyl)sulfane as a pale yellow liquid with some impurity.

1-bromo-3-(isopropylsulfonyl)-5-(prop-1-en-2-yl)benzene was synthesized from (3-bromo-5-(prop-1-en-2-yl)phenyl)(isopropyl)sulfane following the general procedures as described herein.

A mixture of 32 mg (0.106 mmol) of 1-bromo-3-(isopropylsulfonyl)-5-(prop-1-en-2-yl)benzene, 11.2 mmol (0.0954 mmol) of Zn(CN)₂ and 24.3 mg of (0.021 mmol) of Pd(PPh₃)₄ in 3 mL of DMF (degassed) was heated at 100° C. Over a period of 2.5 h, a total of 103 mg of Pd(PPh₃)₄ was added in 3 portions. Ethyl acetate and 10% NH₄OH solution were added, and the organic layer was washed with H₂O and brine. The aqueous layer was extracted with EtOAc (3×), and the combined extracts were dried over Na₂SO₄, filtered, and concentrated. Purification by flash silica gel chromatography (30% EtOAc/hexanes) provided 28.6 mg of 3-(isopropylsulfonyl)-5-(prop-1-en-2-yl)benzonitrile as a yellow solid.

(3-isopropyl-5-(isopropylsulfonyl)phenyl)methanamine was synthesized from 3-(isopropylsulfonyl)-5-(prop-1-en-2-yl)benzonitrile following the general procedure as described above for the isopropenyl acetamide.

### Example 1.1.78

### 4-(aminomethyl)-6-isopropylpyrimidin-2-amine

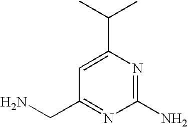

To a stirred slurry of NaH (1.02 g, 26 mmol, washed with anhydrous hexanes) in ether (10 mL) was added methyl methoxyacetate (2.3 mL, 23 mmol) followed by 3-methyl-2-butanone (2.4 mL, 23 mmol) in ether (5 mL). The resulting mixture was stirred for 15 h and became a clear yellow solution. The reaction was quenched with saturated aqueous NH₄Cl. The resulting mixture was extracted with EtOAc (2×20 mL). The combined organic layer was washed with H₂O, brine, dried with Na₂SO₄ and concentrated under reduced pressure to provide 1-methoxy-5-methylhexane-2,4-dione (2.90 g, 80%) as a mixture of keto and enol form.

To a stirred solution of 1-methoxy-5-methylhexane-2,4-dione (1.08 g, 6.8 mmol) in EtOH (15 mL) was added guanidine hydrochloride (1.3 g, 13.7 mmol). 5 min later, a solution of Na₂CO₃ (1.45 g, 13.7 mmol) in H₂O was added and the resulting mixture was heated to reflux for 20 h. The reaction was cooled to room temperature and the solvent was removed. The residue was dissolved in EtOAc (20 mL) and H₂O (20 mL) The layers were separated and the aqueous layer was extracted with EtOAc (2×20 mL). The combined organic layer was washed with brine, dried with Na₂SO₄ and concentrated under reduced pressure. The residue was purified by column chromatography (50% EtOAc in hexanes) to provide 4-isopropyl-6-(methoxymethyl)pyrimidin-2-amine (482 mg, 39%). ¹H NMR (CDCl₃): d 6.43 (s, 1H), 6.11 (s, 2H), 4.16 (s, 2H), 3.26 (s, 3H), 2.56-2.66 (m, 1H), 1.03 (d, J=6.9 Hz, 6H).

To a stirred solution of 4-isopropyl-6-(methoxymethyl)pyrimidin-2-amine (537 mg, 3.0 mmol) in CH₂Cl₂ (30 mL) at 0° C. was added BBr₃ (3 mL of 1.0 M solution, 3 mmol) dropwise. The reaction was quenched with saturated aqueous NaHCO₃ after 4 h. The resulting mixture was extracted with CH₂Cl₂ (2×20 mL). The combined organic layer was washed with brine, dried with Na₂SO₄ and concentrated under reduced pressure to provide (2-amino-6-isopropylpyrimidin-4-yl)methanol as a dark brown oil which was used for next step without further purification. ¹H NMR (CDCl₃): d 6.49 (s, 1H), 5.06 (br, 2H), 4.59 (s, 2H), 2.78-2.87 (m, 1H), 1.25 (m, 6H).

Following standard condition described herein for alcohol to azide transformation, (2-amino-6-isopropylpyrimidin-4-yl)methanol was converted to 4-(azidomethyl)-6-isopropylpyrimidin-2-amine in 66% yield. ¹H NMR (CDCl₃): d 6.57 (s, 1H), 5.20 (br, 2H), 4.27 (s, 2H), 2.79-2.88 (m, 1H), 1.25-1.28 (m, 6H).

Following standard catalytic hydrogenation of azide to amine described herein, 4-(aminomethyl)-6-isopropylpyrimidin-2-amine was obtained from 4-(azidomethyl)-6-isopropylpyrimidin-2-amine in quantitative yield. ¹H NMR (CDCl₃): d 6.44 (s, 1H), 5.63 (br, 2H), 3.70 (s, 2H), 2.71-2.76 (m, 1H), 1.19 (d, J=7.2 Hz, 6H).

### Example 1.1.79

### 3-(aminomethyl)benzonitrile

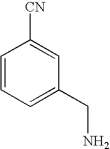

To (3-bromophenyl)methanamine hydrochloride (Aldrich, 4.0 g, 17.97 mmol) in MeOH (35 ml), triethylamine (5.45 g, 53.91 mmol) was added followed by (Boc)₂O (4.7 g, 21.6 mmol). Reaction mixture was stirred overnight at RT and the volatiles are removed on a rotavap under reduced pressure. Then the crude residue was column chromatographed (10% Ethyl acetate/Hexanes) to yield tert-butyl 3-bromobenzylcarbamate in 90% yield.

To the Boc compound tert-butyl 3-bromobenzylcarbamate (1.8 g, 6.3 mmol) in DMF (20 ml), Zn(CN)₂ (443 mg, 3.8 mmol) was added followed by the Pd(PPh₃)₄ and heated at 110° C. for 2.5 h. Then the reaction mixture was diluted with ether, washed with ammonium hydroxide solution, water and brine. Crude residue was column chromatographed (20% ethylacetate/Hexanes) to yield tert-butyl 3-cyanobenzylcarbamate in 70% yield.

To tert-butyl 3-cyanobenzylcarbamate in CH₂Cl₂ (10 ml), TFA (3 ml) was added. After 1 h, volatiles were removed on a rotavap under reduced pressure. Crude residue 3-(aminomethyl)benzonitrile was dissolved in water, basified with 2N NaOH and extracted with CHCl₃ (100 ml) and used without further purification.

### Example 1.1.80

### (5-bromopyridin-3-yl)methanamine

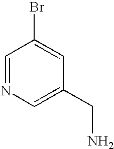

To the cobalt chloride hexahydrate (71 mg, 0.55 mmol) and 5-bromonicotinonitrile (Aldrich, 1 g, 5.5 mmol) in THF:water (19.5:9.25 ml) at 0° C., Sodium borohydride (416 mg, 11.0 mmol) was added in portions with intermittent cooling of the reaction mixture. Once all the sodium borohydride was added, the reaction mixture was stirred for RT for 2 h. Then the reaction mixture was acidified with 3N HCl and stirred at RT for 3.5 h. Then THF was removed under vacuum and the aqueous layer was extracted with ether and ethereal layer was discarded. Then aqueous layer was basified with aqueous NH₄OH solution and extracted repeatedly with CHCl₃ (3×100 ml). Organic layer was dried on anhydrous sodium sulfate and volatiles were removed under vacuum. Column chromatography (MeOH/CHCl₃: 3/97) of the crude residue resulted in (5-bromopyridin-3-yl)methanamine in 25% yield.

### Example 1.1.81

### 2-(3-methoxyphenyl)propan-2-amine

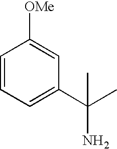

To methyl 2-(3-methoxyphenyl)acetate (Aldrich, 1 g, 5.55 mmol) in THF (10 ml) at 0° C., NaHMDS (5.55 ml, 5.55 mmol) was added. After stirring at 0° C. for 10 min, reaction mixture was stirred at RT for 0.5 h and then MeI (0.4 ml) was added. After 45 min, another 5.55 ml of NaHMDS was added at 0° C. Again after stirring at RT for 0.5 h, 0.5 ml of MeI was added and the reaction mixture was stirred at RT for 2 h. Then the reaction mixture was quenched with ammonium chloride and solvent removed. Then the reaction mixture was diluted with ethyl acetate, acidified with 2N HCl and washed with ether. Aqueous layer was then basified and extracted with CHCl₃. organic layer was dried and evaporated to yield methyl 2-(3-methoxyphenyl)-2-methylpropanoate.

To methyl 2-(3-methoxyphenyl)-2-methylpropanoate (800 mg, 4 mmol) in THF (10 ml) and MeOH (10 ml), aqueous LiOH (excess) was added and the reaction mixture was heated at 60° C. for 7 h. Then volatiles were removed in vacuum and the aqueous layer was extracted with ether. Ether layer was discarded. Then the aqueous layer was acidified and extracted with ethyl acetate. Organic layer was dried with sodium sulfate and volatiles removed under vacuum to yield 2-(3-methoxyphenyl)-2-methylpropanoic acid.

To 2-(3-methoxyphenyl)-2-methylpropanoic acid (330 mg, 1.70 mmol) and triethylamine (191 mg, 1.87 mmol) in t-BuOH (15 ml), DPPA (514 mg, 1.87 mmol) was added and the resultant solution was refluxed for 2 h. Then t-BuOH was removed under vacuum. Crude residue was dissolved in THF (10 ml) and 2N HCl (10 ml) was added and stirred overnight at RT. Then solvent was removed, crude residue dissolved in water and extracted with ether. Aqueous layer was basified with 5N NaOH and extracted with ethyl acetate. Organic layer was dried and evaporated to yield 2-(3-methoxyphenyl)propan-2-amine.

### Example 1.1.82

### 5-(aminomethyl)nicotinonitrile

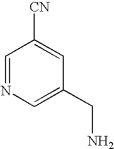

To (5-bromopyridin-3-yl)methanamine (880 mg, 4.70 mmol) in MeOH (20 ml), triethylamine (530 mg, 5.18 mmol) was added followed by (Boc)₂O (1.13 g, 5.18 mmol) at 0° C. Then reaction mixture was allowed to come to RT and stirred for 3 h. Then the volatiles were removed on a rotavap under reduced pressure. Crude residue was purified by column chromatography (ethyl acetate/Hexanes:40/60) to yield the Boc compound 17 in quantitative yield.

To tert-butyl (5-bromopyridin-3-yl)methylcarbamate (384 mg, 1.34 mmol) in DMF (10 ml), Zn(CN)₂ (94 mg, 0.80 mmol) and Pd(PPh₃)₄ were added and heated at 110° C. for 3 h. Then the reaction mixture was cooled to RT, diluted with ether, washed with ammonium hydroxide solution, water and brine. Crude residue was column chromatographed (40% ethylacetate/60% Hexanes) to yield tert-butyl (5-cyanopyridin-3-yl)methylcarbamate in 86% yield.

To tert-butyl (5-cyanopyridin-3-yl)methylcarbamate in CH₂Cl₂ (10 ml), TFA (3 ml) was added. After 1 h, volatiles were removed on a rotavap under reduced pressure. Crude residue was dissolved in water, basified with 2N NaOH and extracted with CHCl₃ (100 ml) to obtain 5-(aminomethyl)nicotinonitrile which was used without further purification.

### Example 1.1.83

### N-(3-(aminomethyl)-5-isopropylphenyl)-N-methylmethanesulfonamide

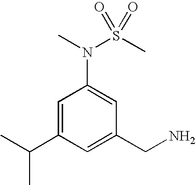

To a stirring solution of 234 mg of 3-(methoxycarbonyl)-5-(N-methylmethylsulfonamido)benzoic acid in 10 mL of THF at r.t. was added 1.2 mL of BH₃.THF (1.0 M in THF). After the solution was stirred at 75° C. for 3 h, 3 mL of acetic acid:H₂O (1:1) was added and stirring was continued until bubbling ceased. Saturated NaHCO₃ solution was added to a pH=7, and the solution was concentrated. Water was added, and the aqueous layer was extracted with EtOAc (2×). The combined extracts were washed with water and brine, dried over Na₂SO₄, filtered, and concentrated. Purification by flash silica gel chromatography (1.5% MeOH/CHCl₃) provided 191 mg of methyl 3-(hydroxymethyl)-5-(N-methylmethylsulfonamido)benzoate as a pale yellow oil in 86% yield.

To a stirring solution of 191 mg (0.699 mmol) of methyl 3-(hydroxymethyl)-5-(N-methylmethylsulfonamido)benzoate in 7 mL of THF at 0° C. was added 1.3-2.0 mL of MeMgBr (3.0 M in Et₂O). After the solution was stirred at 0° C. for about 90 min., the reaction was quenched with saturated NH₄Cl solution and H₂O. The aqueous layer was extracted with EtOAc, and the organic layer was washed with brine, dried over Na₂SO₄, filtered, and concentrated. N-(3-(hydroxymethyl)-5-(2-hydroxypropan-2-yl)phenyl)-N-methylmethanesulfonamide was used in the next reaction without further purification.

To a stirring solution of 189 mg of N-(3-(hydroxymethyl)-5-(2-hydroxypropan-2-yl)phenyl)-N-methylmethanesulfonamide in 7 mL of CH₂Cl₂ was added 600 μL of thionyl chloride (SOCl₂). The solution was stirred for 12 h at rt. and concentrated. The crude product was dissolved in CH₂Cl₂ and sat. NaHCO₃ solution was added. The organic layer was washed with brine, dried over Na₂SO₄, filtered, and concentrated. N-(3-(chloromethyl)-5-(prop-1-en-2-yl)phenyl)-N-methylmethanesulfonamide was used in the next reaction without further purification.

A mixture of crude N-(3-(chloromethyl)-5-(prop-1-en-2-yl)phenyl)-N-methylmethanesulfonamide and 91.2 mg (1.40 mmol) of NaN₃ in 4 mL of DMF was stirred at 70° C. for about 3 h. Water and EtOAc were added, and the organic layer was washed with water (2×) and then brine. The aqueous layer was extracted with EtOAc, and the combined extracts were dried over Na₂SO₄, filtered, and concentrated. Purification by flash silica gel chromatography provided 88.4 mg of N-(3-(azidomethyl)-5-(prop-1-en-2-yl)phenyl)-N-methylmethanesulfonamide as a mixture.

A solution of 88.4 mg of the impure N-(3-(azidomethyl)-5-(prop-1-en-2-yl)phenyl)-N-methylmethanesulfonamide and 90.3 mg (0.344 mmol) of PPh₃ in 6 mL of THF and 0.6 mL of H₂O was stirred at r.t. for 10 h, and then concentrated. Ethyl acetate was added, the solution was dried over Na₂SO₄, filtered, and concentrated. N-(3-(aminomethyl)-5-(prop-1-en-2-yl)phenyl)-N-methylmethanesulfonamide was used without further purification.

A solution of crude N-(3-(aminomethyl)-5-(prop-1-en-2-yl)phenyl)-N-methylmethanesulfonamide, 70 μL (0.502 mmol) of Et₃N and 110 μL (0.479 mmol) of BOC₂O in 7 mL of MeOH was stirred at r.t. for 30 min. and then concentrated. Ethyl acetate and water were added, and the organic layer was washed with water and brine, dried over Na₂SO₄, filtered, and concentrated. Purification by flash silica gel chromatography (40 to 70% EtOAc/hexanes) provided 55.7 mg of pure product and Boc protected N-(3-(aminomethyl)-5-(prop-1-en-2-yl)phenyl)-N-methylmethanesulfonamide which were combined and used in the next reaction.

A solution of Boc protected N-(3-(aminomethyl)-5-(prop-1-en-2-yl)phenyl)-N-methylmethanesulfonamide and 0.8 mL of trifluoroacetic acid (TFA) in 2 mL of CH₂Cl₂ was stirred at r.t. for 1 h. The solution was concentrated and sat. NaHCO₃ was added to a pH=8. The aqueous layer was extracted with the extract of (40 mL CHCl₃: 5 mL of MeOH: 5 mL of H₂O) (3×). The combined extracts were dried over Na₂SO₄, filtered, and concentrated. The crude N-(3-(aminomethyl)-5-(prop-1-en-2-yl)phenyl)-N-methylmethanesulfonamide was used in the next reaction without further purification.

To a stirring solution of crude N-(3-(aminomethyl)-5-(prop-1-en-2-yl)phenyl)-N-methylmethanesulfonamide and 68.3 mg (0.2871 mmol) of CoCl₂.6H₂O in 3 mL of EtOH and 1 mL of THF at 50° C. was added 0.169 g of NaBH₄ in 2 portions. After the mixture was stirred for about 3.5 h, 5 N HCl was added to a pH=1. The mixture was concentrated and (28-30) % NH₄OH solution was added to pH=8. The aqueous layer was extracted with the extract of (40 mL of CHCl₃: 5 mL of H₂O: 5 mL of MeOH) (3×). The combined extracts were dried over Na₂SO₄, filtered, and concentrated. N-(3-(aminomethyl)-5-isopropylphenyl)-N-methylmethanesulfonamide was used in the next reaction without further purification.

### Example 1.1.84

### N-(3-(aminomethyl)-5-isopropylbenzyl)methanesulfonamide

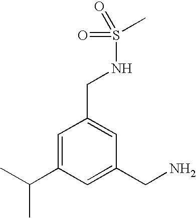

diethyl 5-(azidomethyl)isophthalate was synthesized from diethyl 5-(hydroxymethyl)isophthalate following the general procedures as described herein.

A mixture of 2.2 g (7.93 mmol) of diethyl 5-(azidomethyl)isophthalate and 224 mg of 10% Pd/C in 30 mL of EtOAc was stirred at r.t. under H₂ balloon overnight. The mixture was filtered through Celite and concentrated. The crude product was dissolved in 30 mL of MeOH, 478 mg of 20% Pd(OH)₂ was added, and the mixture was stirred at r.t. under H₂ balloon for about 5 h. The mixture was filtered through Celite and concentrated. The crude diethyl 5-(aminomethyl)isophthalate product was used in the next reaction without further purification.

To a stirring solution of crude diethyl 5-(aminomethyl)isophthalate in 30 mL of CH₂Cl₂ at 0° C. was added 1.2 mL of Et₃N and 0.7 mL of MsCl. The ice bath was removed, and after 2 h at r.t. the solution was concentrated, and EtOAc and H₂O were added. The organic layer was washed with brine, the aqueous layer was extracted with EtOAc, the combined extracts were dried over Na₂SO₄, filtered, and concentrated. Purification by flash silica gel chromatography (1% MeOH/CHCl₃) provided 901 mg of diethyl 5-(methylsulfonamidomethyl)isophthalate as a pale yellow solid in 35% yield.

To a solution of 901 mg (2.73 mmol) of diethyl 5-(methylsulfonamidomethyl)isophthalate in 10 mL of THF and 10 mL of MeOH was added 115 mg of NaOH in 2.7 mL of H₂O. After the solution was stirred at r.t. for 50 h, the solution was concentrated, and H₂O and CHCl₃ were added. The aqueous layer was acidified to pH=1-2 with 1N HCl and extracted with the extract of (40 mL of CHCl₃: 5 mL of MeOH, and 5 mL of H₂O) several times. The combined extracts were dried over Na₂SO₄, filtered, and concentrated to give crude 3-(methoxycarbonyl)-5-(methylsulfonamidomethyl)benzoic acid which was used without further purification.

N-(3-(aminomethyl)-5-isopropylbenzyl)methanesulfonamide was synthesized from 3-(methoxycarbonyl)-5-(methylsulfonamidomethyl)benzoic acid following the general procedures as described herein.

tert-butyl 3-isopropyl-5-(methylsulfonamidomethyl)benzylcarbamate was synthesized and purified from N-(3-(aminomethyl)-5-isopropylbenzyl)methanesulfonamide following the general procedures as described herein.

N-(3-(aminomethyl)-5-isopropylbenzyl)methanesulfonamide was synthesized from the tert-butyl 3-isopropyl-5-(methylsulfonamidomethyl)benzylcarbamate following the general procedure as described above for the N-methyl methylsulfonamide.

### Example 1.1.85

### (5-methyl-1,3,4-oxadiazol-2-yl)methanamine

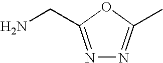

To a stirred solution of Boc-Glycine (1.75 g, 10.0 mmol) in CH₂Cl₂ at 0° C. was added carbonyl imidazole (1.7 g, 10.5 mmol) and the reaction was stirred for 30 min and then acetic hydrzide (740 mg, 10.0 mmol) was added. After 45 min, CBr₄ (6.63 g, 20.0 mmol) and PPh₃ (5.25 g, 20.0 mmol) were added and the reaction was stirred overnight at room temperature. The reaction mixture was concentrated partially and chromatographed (50% EtOAc in hexanes) to provide 2.3 g of tent-butyl (5-methyl-1,3,4-oxadiazol-2-yl)methylcarbamate with some triphenylphosphine oxide as impurity.

To a stirred solution of tert-butyl (5-methyl-1,3,4-oxadiazol-2-yl)methylcarbamate (2.3 g, 10.0 mmol) in CH₂Cl₂ (20 mL), was added TFA (8 mL) and stirred for 1 h. All the solvent was removed and residue is diluted with water and extracted with ether to remove triphenylphosphine oxide. Then the aq. layer was brought P^{H} ˜7 with satd. NaHCO₃, all the water was removed under reduced pressure and the residue was triturated with EtOAc and filtered and concentrated to obtain 900 mg of 20 which is about 90% pure.

### Example 1.1.86

### 3-(aminomethyl)-5-isopropyl-N-methylbenzenesulfonamide

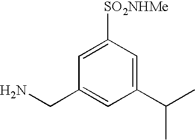

A mixture of 5 g (14.2 mmol) of sodium 4-amino-3,5-dibromobenzenesulfonate in 24 mL of POCl₃ was heated at 120° C. under a CaCl₂ drying tube for 15 min. and 125° C. for about 22 h. Initially ice and cold water were added to the crude product with ice bath cooling, and finally the crude product was poured into ice. Ethyl acetate was added, and the layers were separated. The organic layer was washed with 40 mL of brine, dried over Na₂SO₄, filtered, and concentrated. The 4-amino-3,5-dibromobenzenesulfonic hypochlorous anhydride product was used in the next reaction without further purification.

To a solution of crude 4-amino-3,5-dibromobenzenesulfonic hypochlorous anhydride and 3.7 mL (21.3 mmol) of N,N-diisopropylethyl amine in 50 mL of CH₂Cl₂ at 0° C. was added 10.7 mL of CH₃NH₂ (2.0 M in THF). The mixture was stirred at 0° C. for 10 min. and the ice bath was removed. Stirring was continued with warming to r.t. for about 75 min. and then 3 mL of the CH₃NH₂ solution was added. After about 15 min., H₂O and CHCl₃ were added, and the layers separated. The aqueous layer was extracted with CHCl₃ (2×). The combined extracts were washed with brine, dried over Na₂SO₄, filtered, and concentrated. Purification by flash silica gel chromatography (30-70) % EtOAc/hexanes provided 1.55 g of 2,6-dibromo-4-(methylaminooxysulfonyl)anilineas a pale yellow solid in 35% yield.

To a stirring mixture of 1.55 g (4.49 mmol) of 2,6-dibromo-4-(methylaminooxysulfonyl)aniline in 16 mL of EtOH was added 1.6 mL of H₂SO₄. To the stirring mixture at 90° C. was added 1.2 g (17.4 mmol) of NaNO₂ in 2 portions. The mixture was stirred at 90° C. for 15.75 h, and H₂O and EtOAc were added. The organic layer was washed with brine, dried over Na₂SO₄, filtered, and concentrated. Purification by flash silica gel chromatography (20% EtOAc/hexanes) resulted in 771 mg of O-(3,5-dibromophenylsulfonyl)-N-methylhydroxylamine with impurity.

A mixture of 751 mg (2.29 mmol) of O-(3,5-dibromophenylsulfonyl)-N-methylhydroxylamine, 159 mg (1.35 mmol) of Zn(CN)₂ and 164 mg (0.142 mmol) of Pd(PPh₃)₄, 8 mL of DMF (degassed) was stirred at 80° C. After 70 min., 334 mg of Pd((PPh₃)₄, was added and after 35 min. 414 mg of Pd(PPh₃)₄ was added. After 1 h, EtOAc and 20 mL of 10% NH₄OH (aq) was added, and the layers were separated. The organic layer was washed with 20 mL of 10% NH₄OH (aq) and 20 mL of brine. The combined aqueous layer was extracted with EtOAc. The combined extracts were dried over Na₂SO₄, filtered, and concentrated. Purification by flash silica gel chromatography provided 194 mg of 3-bromo-5-(methylaminooxysulfonyl)benzonitrile as a yellow solid in 31% yield.

3-(methylaminooxysulfonyl)-5-(prop-1-en-2-yl)benzonitrile was synthesized from the 3-bromo-5-(methylaminooxysulfonyl)benzonitrile following the general procedure as described herein for the chloro substituted pyridine. 3-isopropyl-5-(methylaminooxysulfonyl)benzonitrile was synthesized from 3-(methylaminooxysulfonyl)-5-(prop-1-en-2-yl)benzonitrile following the general procedure as described herein for the isopropenyl acetamide.

### Example 1.1.87

### N-methyl-1-(4-((triisopropylsilyloxy)methyl)thiazol-2-yl)methanamine

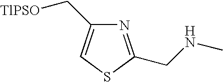

To a DCM solution of thiazol-4-ylmethanol (Combi-Blocks)(1 g, 8.69 mmol) stirred at 0° C., were added TIPSC1 (2.2 mL, 10.43 mmol) and imidazole (1.48 g, 21.72 mmol). The resulting mixture was then warmed to room temperature and stirred for overnight. The reaction was quenched with saturated aqueous NH₄Cl solution, extracted with ethyl acetate three times. The combined organic layers were washed with brine, dried with anhydrous Na₂SO₄, concentrated to a residue which was purified by flash column to give 4-((triisopropylsilyloxy)methyl)thiazole (2 g). ¹H NMR (300 MHz, CDCl₃), d: 8.794 (m, 1H), 7.338 (m, 1H), 5.065 (s, 2H), 1.209 (m, 3H), 1.133 (d, J=6 Hz, 18H).

To a solution of 4-((triisopropylsilyloxy)methyl)thiazole (2 g, 7.367 mmol) in diethyl ether solution (30 mL) at −78° C. was added butyl lithium (1.6 M in hexanes, 5.1 mL). The resulting solution was stirred at the same temperature for one hr, dimethylformate (1.14 mL, 14.734 mmol) was added to the light yellow reaction mixture. The reaction was warmed to room temperature and stirred for 3 hr, then quenched with water, extracted with ethyl acetate three times. The combined organic layers were washed with brine, dried with anhydrous Na₂SO₄, concentrated to a residue which was not purified and identified for the next step. Methylamine (2 M solution in methanol, 10 mL) in the flask at 0° C. was added titanium isopropoxide (2.55 mL, 8.692 mmol) and stirred for 20 minutes, then the above crude aldehyde was added to the reaction and stirred for 3 hr. The reaction mixture was added sodium borohydrate (354 mg, 9.36 mmol) portionwise. After overnight the reaction solvent was removed, diluted with DCM/water. The resulting precipitate was filtered through a celite. The clear liquid was extracted with chloroform three times, dried with anhydrous Na₂SO₄, concentrated to a residue which was purified by flash column to give N-methyl-1-(4-((triisopropylsilyloxy)methyl)thiazol-2-yl)methanamine (1.37 g). ¹H NMR (300 MHz, CDCl₃), d: 7.197 (s, 1H), 4.975 (s, 2H), 4.075 (s, 2H), 2.555 (s, 3H), 1.196 (m, 3H), 1.134 (d, J=5.7 Hz, 18H).

### Example 1.2

### Synthesis of Aldehyde Building Blocks

### Example 1.2.1

### methyl 3-formyl-5-methoxybenzoate

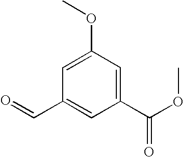

Dimethyl 5-methoxyisophthalate (4 g, 17.84 mmol) in MeOH/water (60 mL/16 mL) was added sodium hydroxide (0.642 g, 16.1 mmol) at 0° C. and the warmed to room temperature and stirred for overnight. After the organic solvent was removed in vacuo, water was added. The obtained suspension was washed with ether twice to remove the starting material. The resulting aqueous layer was acidified to pH ˜4, extracted with EtOAc three times. The combined organic layers were dried in vacuo to produce a white solid. The monoacid (3-methoxy-5-(methoxycarbonyl)benzoic acid) was used directly for next reaction without further purification and identification.

To a stirred solution of 3-methoxy-5-(methoxycarbonyl)benzoic acid (2.6 g, 12.4 mmol) and triethylamine (2.6 mL, 18.6 mmol) in THF (200 mL), isopropyl chloroformate (1 M in toluene, 16 mL) was added at 0° C., and it was stirred at the same temperature for 30 min. After water was added, the mixture was extracted with ether. The combined organic layer was washed with aqueous NaHCO₃ solution, dried over NaSO₄, and concentrated in vacuo. The resulting residue was dissolved in THF (200 mL) and then NaBH₄ (1.4 g) in cold water (40 mL) was added to the solution with stirring at 0° C. After one hour, water was added and the mixture was extracted with EtOAc three times. The combined organic layer was washed with brine, dried over NaSO₄, and concentrated in vacuo, and purified by silica gel chromatography to afford the corresponding alcohol, methyl 3-(hydroxymethyl)-5-methoxybenzoate (2.4 g). ¹H NMR (300 MHz, CDCl₃+CD₃OD), d: 7.648 (s, 1H), 7.503 (s, 1H), 7.162 (s, 1H), 4.747 (s, 2H), 3.947 (s, 3H), 3.888 (s, 3H).

To a solution of methyl 3-(hydroxymethyl)-5-methoxybenzoate (2.18 g, 9.82 mmol) in DCM (100 mL), Dess-Martin periodinane (5 g, 11.79 mmol) was added at rt. After 30 min stirring, the mixture was poured into a mixture of aqueous 1 M Na₂S₂O₃ (30 mL) and aqueous saturated NaHCO₃ (30 mL), and it was extracted with DCM three times. The combined organic layers were concentrated in vacuo and methyl 3-formyl-5-methoxybenzoate residue white solid (˜80% purity) was used directly for the next step reaction without further purification and identification.

### Example 1.2.2

### 5-(2-fluoropropan-2-yl)nicotinaldehyde

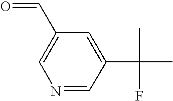

To a suspension of ethyl 5-bromonicotinate (10.0 g, 43.46 mmol) in anhydrous THF (20 mL) was added dropwise to a slurry of LiAlH₄ (1.91 g, 47.81 mmol) in anhydrous THF (200 mL) under argon at −78° C. and the mixture was stirred for 1.5 h at the same temperature, then warmed to r.t. The reaction mixture was added 15 ml of aqueous HCl (1 M) slowly at −78° C., the mixture was then warmed to rt and added anhydrous Na₂SO₄, stirred for overnight. The resulting mixture was filtered through celite and the solvent was removed under reduced pressure to give crude (5-bromopyridin-3-yl)methanol, which was used in the next step without purification. To a solution of (5-bromopyridin-3-yl)methanol (10.6 g, 56.40 mmol) in DMF (30 mL) was added, under argon, imidazole (9.98 g, 146.5 mmol) followed by triisopropylsilyl chloride (TIPS-Cl) (15.53 mL, 73.29 mmol). After stirring for 24 h at room temperature the reaction mixture was evaporated to dryness. Purification by flash chromatography (silica gel, hexanes/EtOAc, 9:1) gave 3-bromo-5-((triisopropylsilyloxy)methyl)pyridine as a colourless oil (11.8 g, 81% overall). ¹H NMR (CDCl₃): d: 1.00-1.237 (m, 21H), 4.837 (s, 2H), 7.849 (s, 1H), 8.480 (s, 1H), 8.556 (s, 1H).

Toluene (30 mL) in a 3-necked flask was cooled down to −78° C. n-BuLi (1.6 M in hexane, 8.35 mL, 0.13.35 mmol) was slowly added to the toluene. After 10 minutes a solution of the 3-bromo-5-((triisopropylsilyloxy)methyl)pyridine (4.0 g, 11.62 mmol) in toluene (30 mL) was added dropwise. A yellow solid precipitated. The resulting slurry was aged for 15-30 min, then THF (20 mL) was added slowly, keeping the internal temperature at <−50° C. The mixture was aged for 15 min, then acetone (1.7 mL, 23.24 mmol) was added over 2 min. The solids dissolved and a brown homogeneous solution was obtained. The reaction solution was warmed to rt and quenched with saturated aqueous NH₄Cl and diluted with EtOAc. The phases were separated and aqueous layer was extracted with EtOAc twice. The organic layers were combined and concentrated to dryness. Purification by flash chromatography (silica gel, hexanes/EtOAc) gave 2-(5-((triisopropylsilyloxy)methyl)pyridin-3-yl)propan-2-ol as a light yellow solid (3.56 g, 96%). ¹H NMR (CDCl₃): d: 1.250-1.055 (m, 21H), 1.606 (s, 6H), 4.865 (s, 2H), 7.830 (s, 1H), 8.458 (s, 1H), 8.621 (s, 1H).

To 2-(5-((triisopropylsilyloxy)methyl)pyridin-3-yl)propan-2-ol (1.1 g, 3.4 mmol) in DCM (30 mL) at −78° C. was added diethylaminosulfur trifluoride (DAST) (0.67 mL, 5.1 mmol) and stirred at the same temperature for 1 hr, then warmed to 0° C. for 3 hrs. The resulting mixture was quenched with MeOH and saturated aqueous NaHCO₃. The mixture was extracted with EtOAc three times and dried with anhydrous NaSO₄, filtered and concentrated to dryness. Careful purification by flash chromatography (silica gel, hexanes/EtOAc) gave the desired fluoride (3-(2-fluoropropan-2-yl)-5-((triisopropylsilyloxy)methyl)pyridine) as a light yellow solid (0.43 g). ¹H NMR (CDCl₃): d: 1.127 (d, J=6.3 Hz, 18H), 1.204 (m, 3H), 1.712 (s, 3H), 1.786 (s, 3H), 4.922 (s, 2H), 7.781 (s, 1H), 8.566 (s, 2H). 3-(2-fluoropropan-2-yl)-5-((triisopropylsilyloxy)methyl)pyridine was deprotected with excess aqueous HF in THF to provide (5-(2-fluoropropan-2-yl)pyridin-3-yl)methanol as a white solid. (5-(2-fluoropropan-2-yl)pyridin-3-yl)methanol was then oxidized to 5-(2-fluoropropan-2-yl)nicotinaldehyde using standard Swern Oxidation conditions.

### Example 1.2.3

### 5-tert-butylnicotinaldehyde

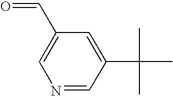

To a suspension of ethyl 5-bromonicotinate (Alfa Aesar, 10.0 g, 43.46 mmol) in anhydrous THF (20 mL) was added dropwise to a slurry of LiAlH₄ (1.91 g, 47.81 mmol) in anhydrous THF (200 mL) under argon at −78° C. and the mixture was stirred for 1.5 h at the same temperature, then warmed to r.t. The reaction mixture was added 15 ml of aqueous HCl (1 M) slowly at −78° C., the mixture was then warmed to rt and added anhydrous Na₂SO₄, stirred for overnight. The resulting mixture was filtered through celite and the solvent was removed under reduced pressure to give crude (5-bromopyridin-3-yl)methanol, which was used in the next step without purification.

To a solution of (5-bromopyridin-3-yl)methanol (10.6 g, 56.40 mmol) in DMF (30 mL) was added, under argon, imidazole (9.98 g, 146.5 mmol) followed by triisopropylsilyl chloride (TIPSCl, 15.53 mL, 73.29 mmol). After stirring for 24 h at room temperature the reaction mixture was evaporated to dryness. Purification by flash chromatography (silica gel, hexanes/EtOAc, 9:1) gave the TIPS ether, (3-bromo-5-((triisopropylsilyloxy)methyl)pyridine) as a colourless oil (11.8 g, 81% overall). ¹H NMR (CDCl₃): d: 1.00-1.237 (m, 21H), 4.837 (s, 2H), 7.849 (s, 1H), 8.480 (s, 1H), 8.556 (s, 1H).

A mixture of anhydrous CuCN (3.41 g, 38.03 mmol) in 150 mL of anhydrous THF, and ethereal tert-butylmagnesium bromide (38 mL, 76.1 mmol) was stirred under N₂ at −78° C. for 20 min. 3-bromo-5-((triisopropylsilyloxy)methyl)pyridine (3.274 g, 9.51 mmol) in THF was added, and the reaction mixture was stirred for 2-3 h at −78° C. and then overnight at room temperature. The reaction mixture was quenched by dropwise addition of saturated aqueous NH₄OH and the pH was adjusted to 10 by using 1 M aqueous NaOH, and the resulting solution was extracted with extracted with EtOAc (3×75 mL). The combined organic extracts were dried (Na₂SO₄), and concentrated under vacuum to get a residue which was purified by flash column chromatography to give 3-tert-butyl-5-((triisopropylsilyloxy)methyl)pyridine (0.7 g). ¹H NMR shows the products contain the desired product and bromine-removed product. ¹H NMR (CDCl₃): d: 1.124 (m, 22H), 1.369 (s, 5H), 4.878 (s, 2H), 7.298 (m, 0.5H), 7.738 (m, 1H), 8.414 (s, 0.6H), 8.607 (m, 1.5H).

3-tert-butyl-5-((triisopropylsilyloxy)methyl)pyridine (0.7 g, 2.637 mmol) was dissolved in HCl in methanol (42 mL, 1.25M, 52.73 mmol) at room temperature and stirred for overnight. The solvent was removed under vacuum, dissolved in chloroform and washed with saturated aqueous Na₂CO₃. The resulting organic solvent was dried, concentrated to give a residue which was purified by flash column chromatography to give pure (5-tert-butylpyridin-3-yl)methanol (0.2 g). ¹H NMR (CDCl₃): d: 1.280 (s, 9H), 4.663 (s, 2H), 7.730 (s, 1H), 8.225 (s, 1H), 8.368 (s, 1H).

Oxalyl chloride (158 μL, 1.819 mmol) in methylene chloride (10 mL) was placed in a two-necked flask at −78° C., followed by the addition of dimethyl sulfoxide (129 μL, 1.819 mmol). Stirring was continued for 20 min, followed by addition of (5-tert-butylpyridin-3-yl)methanol (0.2 g, 1.21 mmol) in methylene chloride (10 mL). After the mixture was stirred at −78° C. for additional 20 min, triethylamine (0.59 mL, 4.24 mmol) was added. The cooling bath was removed and the suspension was allowed to warm to room temperature. Water (50 mL) was added, the yellow organic layer was separated, and the aqueous layer was extracted with methylene chloride (3×30 mL). The combined organic solution was dried and concentrated to give 5-tert-butylnicotinaldehyde as an orange-yellow liquid which was used directly for next step without further purification.

### Example 1.2.4

### 5-(1,1-difluoroethyl)nicotinaldehyde

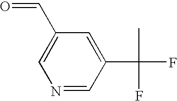

To 1-(5-bromopyridin-3-yl)ethanone (2.95 g, 14.75 mmol) in flask was added [Bis(2-methoxyethyl)amino]sulfur trifluoride (4.1 mL, 22.12 mmol) and heated to 80° C. The resulting mixture was stirred at this temperature for overnight. The reaction was cooled to room temperature and quenched with MeOH and saturated aqueous NaHCO₃. The mixture was extracted with methylene chloride three times and dried with anhydrous NaSO₄, filtered and concentrated to dryness. Careful purification by flash silica chromatography provided 3-bromo-5-(1,1-difluoroethyl)pyridine as a light yellow solid (1.5 g). ¹H NMR (CDCl₃): d: 1.993 (t, J=18.3 Hz, 3H), 8.002 (s, 1H), 8.720 (s, 1H), 8.794 (s, 1H).

3-bromo-5-(1,1-difluoroethyl)pyridine was converted to 5-(1,1-difluoroethyl)nicotinaldehyde using BuLi in DMF under similar conditions as described herein and used for the next step without further purification.

### Example 1.2.5

### 3-(1,1-difluoroethyl)benzaldehyde

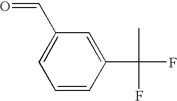

3-(1,1-difluoroethyl)benzonitrile was synthesized from 3-acetylbenzonitrile following the method described for 3-bromo-5-(1,1-difluoroethyl)pyridine. A solution of 3-(1,1-difluoroethyl)benzonitrile (1.6 g, 9.57 mmol) in CH₂Cl₂ (25 mL) was cooled to 0° C. and was treated dropwise with a 1 M solution of DIBAL in hexanes (11.5 mL, 11.2 mmol). The mixture was allowed to slowly warm to room temperature. The reaction was monitored by TLC. After 3 h, the reaction mixture was poured into a beaker containing crushed ice and 6 N HCl. The mixture was stirred for about 1 h. The layers were separated and the aqueous phase was extracted with CH₂Cl₂. The combined organic layer was washed with aqueous NaHCO₃ followed by water. The organic layer was dried (Na₂SO₄), concentrated, and silica chromatographed to afford 3-(1,1-difluoroethyl)benzaldehyde as a light yellow oil, which was used directly for next step without further purification and identification.

### Example 1.2.6

### 3-hydroxy-5-isopropylbenzaldehyde

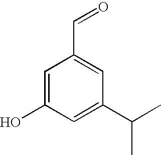

To a stirring solution of 5 g (18.9 mmol) of 3,5-dibromobenzaldehyde in 40 mL of MeOH and 15 mL of THF was added 3.2 mL (29.2 mmol) of HC(OMe)₃ followed by 278 mg (1.46 mmol) of p-TsOH.H₂O. The solution was stirred at r.t. for about 14 h and concentrated. Water was added to the crude product, and the aqueous layer was extracted with EtOAc. The organic layer was washed with brine, dried over Na₂SO₄, filtered, and concentrated. Purification by flash silica gel chromatography (5% EtOAc/hexanes) provided a mixture of the aldehyde and 1,3-dibromo-5-(dimethoxymethyl)benzene. Hexanes was added to separate the products by solubility, but the dimethyl acetal was still not pure and used without further purification.

To a stirring solution of 23 mL of n-BuLi (1.6 M in hexanes) in 20 mL of THF at −78° C. was added 5.31 g of 1,3-dibromo-5-(dimethoxymethyl)benzene in 80 mL of THF dropwise over a period of about 55 min. After 50 min., 3.2 mL (28.7 mmol) of B(OMe)₃ was added, and the solution was stirred at −78° C. for 20 min. The cold bath was removed and stirring continued with gradual warming to r.t. After 1 h, 1 N HCl (45 mL) was added, and the aqueous layer was extracted with EtOAc. The extract was washed with brine, dried over Na₂SO₄, filtered, and concentrated. To a stirring mixture of crude product in 70 mL of 1N NaOH at 0° C. was added 14.5 mL of H₂O₂ (30 wt % in H₂O) dropwise. After 25 min., 5 N HCl was added to a pH=1, and EtOAc and H₂O were added. The organic layer was washed with brine, dried over Na₂SO₄, filtered, and concentrated. Purification by flash silica gel chromatography (15% EtOAc/hexanes) provided 1.92 g of 3-bromo-5-(dimethoxymethyl)phenol as a pale yellow solid with some impurity.

3-(dimethoxymethyl)-5-(prop-1-en-2-yl)phenol was synthesized from 3-bromo-5-(dimethoxymethyl)phenol following the general procedures as described herein.

A mixture of 554 mg of 3-(dimethoxymethyl)-5-(prop-1-en-2-yl)phenol and 55.2 mg of 10% Pd/C in 10 mL of MeOH and 10 mL of EtOAc was stirred at r.t under H₂ balloon for 4 h. The mixture was filtered through Celite and concentrated. 3-(hydroxymethyl)-5-isopropylphenol was used in the next reaction without further purification.

To a stirring solution of 3-(hydroxymethyl)-5-isopropylphenol in 10 mL of CH₂Cl₂ was added 1.23 g (5.69 mmol) of PCC. The mixture was stirred for 4.5 h, Et₂O was added, and the mixture was stored in a refrigerator. The solvent was concentrated, and the crude product was purified by flash silica gel chromatography (15% EtOAc/hexanes) to provide 218 mg of 3-hydroxy-5-isopropylbenzaldehyde as a pale yellow solid.

### Example 1.2.7

### 3-formyl-5-isopropylphenyl morpholine-4-carboxylate

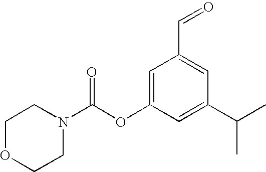

To a stirring solution of 91.9 mg (0.634 mmol) of 3-hydroxy-5-isopropylbenzaldehyde in 5 mL of CH₂Cl₂ at r.t. was added 220 μL (1.92 mmol) of 4-morpholinecarbonyl chloride and 0.5 mL (3.59 mmol) of Et₃N. After 3 h, sat. NaHCO₃ (15 mL) was added, and the aqueous layer was extracted with CHCl₃. The organic layer was washed with brine, dried over Na₂SO₄, filtered, and concentrated. Purification by flash silica gel chromatography (20% EtOAc/hexanes) provided 38.8 mg of 3-formyl-5-isopropylphenyl morpholine-4-carboxylate as a yellow oil in 24% yield.

### Example 1.2.8

### 5-acetylnicotinaldehyde

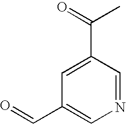

To a stirred solution of 3-(dimethoxymethyl)-5-(prop-1-en-2-yl)pyridine (7.1 mL) in CH₂Cl₂ (30 mL) at 0° C. was added CrO₃ (4.4 g, 44 mmol). The resulting mixture was stirred for 1 h and 10 (854 mg, 4.4 mmol) was added. The stirring was continued for 5 d and the mixture was filtered through a pad of Celite. The filtrate was concentrated and diluted with EtOAc. The organic layer was washed with NaHCO₃, NH₄Cl, brine and dried over Na₂SO₄. The solvent was removed and the residue was purified by column chromatography (60% EtOAc in hexanes) to provide 1-(5-(dimethoxymethyl)pyridin-3-yl)ethanone as a yellow oil.

To a stirred solution of 1-(5-(dimethoxymethyl)pyridin-3-yl)ethanone (214 mg, 1.1 mmol) in CH₂Cl₂ (5 mL) was added TFA (2 mL). The resulting mixture was stirred for 24 h and the solvent was removed. The residue was dissolved in CHCl₃ and saturated aqueous NaHCO₃. The layers were separated and the organic layer was washed with brine and dried over Na₂SO₄. The solvent was removed to provide 5-acetylnicotinaldehyde (112 mg) as a yellow solid.

### Example 1.2.9

### 3-formyl-5-(prop-1-en-2-yl)pyridine 1-oxide

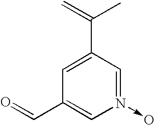

To a stirred solution of 3-(dimethoxymethyl)-5-(prop-1-en-2-yl)pyridine (178 mg, 0.92 mmol) in CH₂Cl₂ (10 mL) was added m-CPBA (227 mg, 1.0 mmol). The reaction mixture was stirred for 2 h and quenched with saturated aqueous NaHCO₃. The layers were separated and the organic layer was washed with brine and dried over Na₂SO₄. The solvent was removed to provide 3-(dimethoxymethyl)-5-(prop-1-en-2-yl)pyridine 1-oxide (209 mg), which was converted to 3-formyl-5-(prop-1-en-2-yl)pyridine 1-oxide as described herein.

### Example 1.2.10

### 3-formyl-5-(prop-1-en-2-yl)benzonitrile

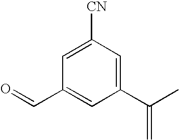

To the 3-formylbenzonitrile (1.3 gm, 10 mmol) in sulfuric acid (4.5 ml) at 60° C., N-bromosuccinimide (2.14 g, 12 mmol) was added in 3 portions. After 2 h, the reaction mixture was cooled, diluted with cold water and filtered. Filter cake was washed with hexane. Volatiles were removed under reduced pressure and the crude residue (3-bromo-5-formylbenzonitrile) was carried to the next step without further purification.

To 3-bromo-5-formylbenzonitrile (420 mg, 2 mmol), isopropenyl potassium trifluoroborate (296 mg, 2 mmol) and triethyl amine (0.42 ml) in 2-propanol and water (20 ml) in 2:1 ratio, PdCl₂(dppf) (65 mg, 0.08 mmol) was added and the reaction mixture was refluxed for 5 h. Then the reaction mixture was cooled, diluted with ether, washed with water, brine and dried. Crude residue was column chromatographed (60% ethylacetate:40% hexane) to yield a white solid of 3-formyl-5-(prop-1-en-2-yl)benzonitrile.

### Example 1.2.11

### 3-formyl-5-isopropylbenzonitrile

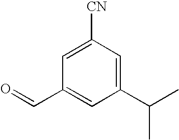

To 3-formyl-5-(prop-1-en-2-yl)benzonitrile (300 mg, 1.75 mg) in ethyl acetate (3 ml), ethanol (3 ml) mixture, 10% Pd/C (30 mg) was added and the reaction mixture was stirred under hydrogen atmosphere and under balloon pressure for 7 h. Then the reaction mixture was filtered and the solvent was evaporated to yield the aldehyde 3-formyl-5-isopropylbenzonitrile.

### Example 1.2.12

### 4-(trifluoromethyl)picolinaldehyde

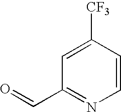

To 2-bromo-4-(trifluoromethyl)pyridine (700 mg, 3.1 mmol) in ether (30 ml) at −78° C., BuLi (1.6M in hexanes, 2.13 ml) was added. After 40 min, DMF (340 mg) was added and the reaction mixture was stirred at −78° C. for a further 45 minutes and the reaction mixture was allowed to come to rt over a period of 1 h. Then the reaction mixture was quenched with solid ammonium chloride and partitioned between water and ether. Organic layers were dried and evaporated to provide 4-(trifluoromethyl)picolinaldehyde as a crude residue which was carried to the next step without further purification.

### Example 1.2.13

### 5-(isopropylamino)nicotinaldehyde

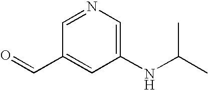

To 5-bromonicotinaldehyde (1.5 gm, 8.06 mmol) in DMF (5 ml), N,N-diethylsalicylidiamide (311 mg, 1.61 mmol), copper iodide (77 mg, 0.403 mmol), potassium phosphate (3.42 gm, 16.12 mmol) and isopropylamine (715 mg, 1.21 mmol) were added and the reaction mixture was heated overnight at 90° C. Then reaction mixture was diluted with ether and filtered. Ether layer was washed with water, brine and dried. Crude residue was column cromatographed (40% ethylacetate/60% Hexanes) to yield 5-(isopropylamino)nicotinaldehyde.

### Example 1.2.14

### 3-bromo-5-(trifluoromethyl)benzaldehyde

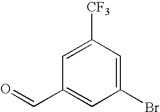

To 3-(trifluoromethyl)benzaldehyde (5 gm, 28.72 mmol) in sulfuric acid (13.5 ml) at 60° C., N-bromosuccinimide (6.13 g, 34.45 mmol) was added in 3 portions. After 2 h, the reaction mixture was cooled, diluted with cold water and filtered. Filter cake was washed with hexane. Volatiles were removed under reduced pressure and the crude residue of 3-bromo-5-(trifluoromethyl)benzaldehyde was carried to the next step without purification.

### Example 1.2.15

### 3-formyl-5-(trifluoromethyl)benzonitrile

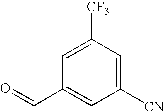

To 3-bromo-5-(trifluoromethyl)benzaldehyde (1.0 gm, 3.95 mmol) in DMF (10 ml), zinc cyanide (278 mg, 2.37 mmol) was added followed by Pd(PPh₃)₄ (365 mg, 0.32 mmol) was added and heated at 90° C. for 5 h. Then the reaction mixture was cooled, diluted with ether and quenched with aqueous ammonium hydroxide solution. Then the reaction mixture was partitioned between water and ether. Organic layer was dried, evaporated and column purified (10% ethylacetate/90% hexanes) to yield 400 mg of 3-formyl-5-(trifluoromethyl)benzonitrile.

### Example 1.2.16

### 3-(pyridin-4-yl)-5-(trifluoromethyl)benzaldehyde

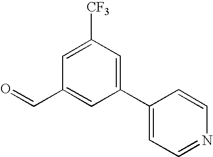

To 3-bromo-5-(trifluoromethyl)benzaldehyde (1.0 g, 3.95 mmol) in 1,4-dioxane (15 ml), pyridine-4-boronic acid) (583 mg, 4.74 mmol), sodium carbonate (2M aqueous solution) (1.67 gms in 7.9 ml water) and Pd(PPh₃)₄ 450 mg, 0.39 mmol) was added and heated at 90° C. for 5 h. Then reaction mixture was diluted with ether, washed with water, brine and dried. Volatiles were removed under vacuum and the crude residue was column chromatographed (50% ethylacetate/50% hexanes) to yield 250 mg of 3-(pyridin-4-yl)-5-(trifluoromethyl)benzaldehyde.

### Example 1.2.17

### N-(3-formyl-5-(trifluoromethyl)phenyl)acetamide

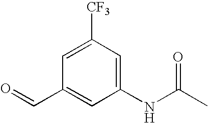

To the mixture of 3-(trifluoromethyl)benzaldehyde (10 g, 57.43 mmol) and sulfuric acid (20 ml) at 0° C., nitric acid (2.5 ml) was added and the reaction was stirred at 0° C. for 1 h, allowed to come to rt for 4 h and heated at 50° C. for 8 h. The reaction mixture was poured into ice water and extracted with ethyl acetate. Combined extracts were washed with water, bicarbonate and brine. Crude residue of 3-nitro-5-(trifluoromethyl)benzaldehyde was dried over sodium sulfate and volatiles removed in vacuum.

To the crude 3-nitro-5-(trifluoromethyl)benzaldehyde (2.4 g, 10.95 mmol) in MeOH (20 ml), P-TSA (1.1 g, 5.61 mmol) was added followed by trimethyl orthoformate (3.5 g, 33.02 mmol). After refluxing for 24 h, solvent was removed under vacuum. Then the resulting residue was diluted with ethyl acetate and basified with saturated aqueous sodium carbonate solution. Aqueous layer was extracted with ethyl acetate. Organic layer was dried and evaporated and the crude residue of 1-(dimethoxymethyl)-3-nitro-5-(trifluoromethyl)benzene was carried to the next step without any further purification.

To 1-(dimethoxymethyl)-3-nitro-5-(trifluoromethyl)benzene (1.9 gm, 7.6 mmol) in ethanol (20 ml), 10% Pd/C (200 m) was added, stirred under balloon pressure for 5 h. Then the reaction mixture was filtered and volatile were removed under vacuum to provide 3-(dimethoxymethyl)-5-(trifluoromethyl)aniline.

To 3-(dimethoxymethyl)-5-(trifluoromethyl)aniline in dichloromethane (5 ml) at 0° C., triethyl amine (0.2 ml) was added followed by acetyl chloride. Reaction mixture was stirred at rt for 2 h. Then dichloromethane was removed under reduced pressure. Reaction mixture was diluted with ether, washed with water, brine and dried. Crude residue was column chromatographed to yield 170 mg of the amide. Then solvent was removed and the crude residue was column purified (40% ethylacetate/60% hexanes) to yield 100 mg of N-(3-(dimethoxymethyl)-5-(trifluoromethyl)phenyl)acetamide.

To the acetal N-(3-(dimethoxymethyl)-5-(trifluoromethyl)phenyl)acetamide (170 mg, 0.65 mmol), TFA (5 ml) was added at 0° C., and the reaction mixture was stirred at rt for 16 h. Then volatiles were removed under vacuum and the reaction mixture was diluted with ether. Ether layer was washed with aqueous sodium bicarbonate solution, water, brine and dried. Organic layer was dried and evaporated to provide N-(3-formyl-5-(trifluoromethyl)phenyl)acetamide. The crude residue was carried to the next step without any further purification.

### Example 1.2.18

### 3-(methylamino)-5-(trifluoromethyl)benzaldehyde

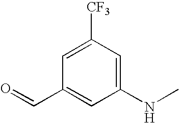

To 3-(dimethoxymethyl)-5-(trifluoromethyl)aniline (440 mg, 2.0 mmol) in methanol (5 ml) at 0° C., triethyl amine (0.2 ml) was added followed by (Boc)₂O (436 mg, 2.0 mmol). Then solvent was removed and the crude residue was column purified (40% ethylacetate/60% hexanes) to yield 100 mg of tert-butyl 3-(dimethoxymethyl)-5-(trifluoromethyl)phenylcarbamate.

To tert-butyl 3-(dimethoxymethyl)-5-(trifluoromethyl)phenylcarbamate (125 mg, 0.37 mmol) in DMF (5 ml) at 0° C., sodium hydride (22 mg, 0.56 mmol) was added. After stirring at rt for 0.5 h, methyl iodide (263 mg, 1.85 mmol) was added and stirred overnight at rt. Reaction mixture was then cooled, quenched with methanol, extracted with ether. Organic layer was dried and evaporated and the crude residue was column purified (10% ethylacetate/90% hexanes) to yield 110 mg of tert-butyl 3-(dimethoxymethyl)-5-(trifluoromethyl)phenyl(methyl)carbamate.

To tert-butyl 3-(dimethoxymethyl)-5-(trifluoromethyl)phenyl(methyl)carbamate (110 mg, 0.31 mmol), TFA (3 ml) was added at 0° C., and the reaction mixture was stirred at rt for 16 h. Then volatiles were removed under vacuum and the reaction mixture was diluted with ethyl acetate. Ethyl acetate layer was washed with aqueous sodium bicarbonate solution, water, brine and dried. Organic layer was dried and evaporated to yield the aldehyde 3-(methylamino)-5-(trifluoromethyl)benzaldehyde. The crude residue was carried to the next step without any further purification.

### Example 1.2.19

### 5-(prop-1-en-2-yl)nicotinaldehyde

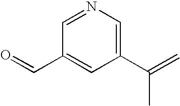

To 3-(dimethoxymethyl)-5-(prop-1-en-2-yl)pyridine (2.2 g, 11.3 mmol), TFA (5.2 ml, 67.8 mmol) was added at 0° C., and the reaction mixture was stirred at rt for 16 h. Then volatiles were removed under vacuum and the reaction mixture was diluted with ether. Ether layer was washed with aqueous sodium bicarbonate solution, water, brine and dried. The organic layer was dried and evaporated to provide 5-(prop-1-en-2-yl)nicotinaldehyde.

### Example 1.2.20

### 3-formyl-5-isopropylphenyl acetate

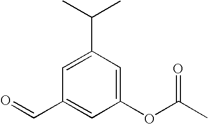

A solution of 33.2 mg (0.202 mmol) of 3-hydroxy-5-isopropylbenzaldehyde and 50 μL (0.53 mmol) of Ac₂O in 3 mL of pyridine was stirred at r.t. for 70 min. The solution was concentrated, water and EtOAc were added, and the layers were separated. The organic layer was washed with brine, dried over Na₂SO₄, filtered, concentrated, and purified by flash silica gel chromatography (20% EtOAc/hexanes) (neutral silica gel) to provide 47.4 mg of 3-formyl-5-isopropylphenyl acetate as a yellow oil with some impurity.

### Example 1.2.21

### 5-(hydroxymethyl)-2-methylbenzaldehyde

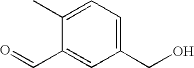

To a stirring mixture of 23.0 g (90.6 mmol) of I₂ and 5.4 mL (45.5 mmol) of tert-butylnitrite in 40 mL of CH₃CN at 35° C. was added 5.0 g (30.3 mmol) of methyl 3-amino-4-methylbenzoate in 4 portions. Stirring was continued with cooling to r.t. in the dark (light off only). After 2.5 h, 400 mL of Na₂SO₃ in H₂0 was added gradually. The layers were separated, and the organic layer was washed with 80 mL of brine, dried over Na₂SO₄, filtered, and concentrated. Purification by flash silica gel chromatography (5% EtOAc/hexanes) provided 5.7 g of methyl 3-iodo-4-methylbenzoate as a red liquid with some impurity in approximately 68% yield.

To a solution of 5.7 g of methyl 3-iodo-4-methylbenzoate in 35 mL of DMF (degassed) was added 1.94 g (16.5 mmol) of Zn(CN)₂ and 1.58 g (1.37 mmol) of Pd(PPh₃)₄. The mixture was stirred at 80° C. in the dark (with the light off) for 6 h. To the mixture was added 50 mL of 10% NH₄OH (aq.) and EtOAc. The organic layer was washed with 50 mL of water and 30 mL of brine, and the combined aqueous layers were extracted with EtOAc. The combined extracts were dried over Na₂SO₄, filtered, and concentrated. Purification by flash silica gel chromatography (10% EtOAc/hexanes) provided about 3 g of methyl 3-cyano-4-methylbenzoate as a pale yellow solid.

To a stirring solution of 27 mL of DIBAL-H (1.5 M solution in toluene) in 10 mL of CH₂Cl₂ at −78° C. was added 1.32 g (7.54 mmol) of methyl 3-cyano-4-methylbenzoate in 35 mL of CH₂Cl₂ dropwise over a period of 30 min. After stirring for about 40 min. at −78° C., the solution was allowed to warm to r.t. After 75 min., the solution was cooled to −78° C. and 10 mL of H₂O and 16 mL of 1N HCl were added. The cold bath was removed, and the mixture was allowed to warm to r.t. Concentrated HCl (5-6 mL) was added and stirring was continued for 1.5 h. Chloroform was added, the layers were separated, and the organic layer was washed with 20 mL of brine. The combined aqueous layers were extracted with chloroform (4×). The combined extracts were dried over Na₂SO₄, filtered, and concentrated. Purification by flash silica gel chromatography (50% EtOAc/hexanes) provided the 800 mg of 5-(hydroxymethyl)-2-methylbenzaldehyde as a yellow oil with some impurity.

### Example 1.2.22

### methyl 5-formyl-2-methylbenzoate

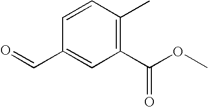

To a stirring solution of 800 mg of 5-(hydroxymethyl)-2-methylbenzaldehyde in 20 mL of MeOH was added 3.4 g (5.5 mmol) of Oxone. The mixture was stirred at r.t. for 3.75 h and 2 mL of MeOH was added. After 2.5 h, 15 mL of 1N HCl, 45 mL of H₂O, and EtOAc were added, and the layers separated. The aqueous layer was extracted, the combined extracts were washed with brine, dried over Na₂SO₄, filtered, and concentrated. Purification by flash silica gel chromatography (30% EtOAc/hexanes) provided 377 mg of methyl 5-(hydroxymethyl)-2-methylbenzoate as a yellow oil with some impurity.

A solution of 377 mg of methyl 5-(hydroxymethyl)-2-methylbenzoate and 0.8 mL (9.17 mmol) of oxalyl chloride in 20 mL of CH₂Cl₂ was cooled to −78° C. To this solution was added 1.1 mL (15.5 mmol) of DMSO dropwise, and after stirring for 10 min., 3.2 mL of Et₃N was added and stirring was continued at −78° C. for 10 min. The dry ice-acetone bath was removed, and the mixture was allowed to warm to r.t. and after 2 h, 30 mL of sat. NH₄Cl solution was added. The organic layer was washed with 25 mL of brine, dried over Na₂SO₄, filtered, and concentrated. Purification by flash silica gel chromatography (10% EtOAc/hexanes) provided 268 mg of methyl 5-formyl-2-methylbenzoate as a red liquid in 71% yield with some impurity.

### Example 1.2.23

### 3-formyl-5-isopropylphenyl dimethyl phosphate

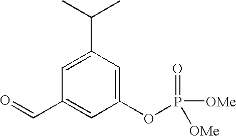

To a stirring solution of 172 mg (1.05 mmol) of 3-hydroxy-5-isopropylbenzaldehyde, 160 μL (1.98 mmol) of pyridine, and 1.6 mL of THF in 8 mL of CH₂Cl₂ was added 620 mg (2.44 mmol) of I₂ and 280 μL (2.37 mmol) of P(OMe)₃ in 5 mL of CH₂Cl₂. The mixture was stirred at r.t. in the dark (light off only) for 2.75 days. Chloroform and water were added, and the layers separated. The organic layer was washed with 20 mL of water and 20 mL of brine, dried over Na₂SO₄, filtered, and concentrated. Purification by flash silica gel chromatography (60% EtOAc/hexanes) (neutral silica gel) provided 40.1 mg of 3-formyl-5-isopropylphenyl dimethyl phosphate in 14% yield.

### Example 1.2.24

### 5-(1-methoxyprop-1-en-2-yl)nicotinaldehyde

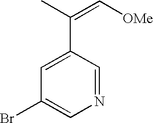

To a solution of methoxymethyltriphenylphosphonium chloride (20.57 g, 60 mmol) in THF at −78° C. was added slowly butyl lithium (1.6 M in hexanes, 37.5 mL). The resulting mixture was further stirred for 45 min to room temperature. After the reaction was cooled down to −78° C., 1-(5-bromopyridin-3-yl)ethanone (Aldich) (8 g, 40 mmol) in THF was added to the reaction mixture. The resulting solution was stirred from −78° C. to room temperature for overnight, then quenched with saturated aqueous NH₄Cl solution, and extracted with diethyl ether three times. The combined organic layers were washed with brine, dried with anhydrous Na₂SO₄, and concentrated to a residue which was purified by flash column to give 3-bromo-5-(1-methoxyprop-1-en-2-yl)pyridine (E/Z mixture, 5 g). ¹H NMR (300 MHz, CDCl₃), d: 8.769 (m, 0.5H), 8.491 (m, 1.5H), 8.143 (m, 0.4H), 7.737 (m, 0.6H), 6.517 (m, 0.6H), 6.271 (m, 0.4H), 3.804, 3.773 (ss, 3H), 1.949 (m, 3H). The aldehyde was synthesized using the methods described herein.

### Example 1.3

### Synthesis of Isophthalate Building Blocks

### Example 1.3.1

### 3-(methoxycarbonyl)-5-(N-methylmethylsulfonamido)benzoic acid

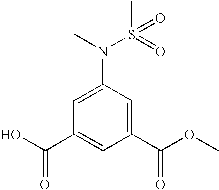

To a stirred solution of dimethyl 5-aminoisophthalate (2.09 g, 10 mmol) in dichloromethane (30 mL), pyridine (2.43 mL, 30 mmol) was added at room temperature. At 0° C., methanesulfonyl chloride (0.86 mL, 11 mmol) was added and the resulting mixture was stirred overnight at room temperature. The reaction mixture was then concentrated under reduced pressure and ethyl acetate (50 mL) was added. The resulting white precipitate was filtered and washed with hexanes to give dimethyl 5-(methylsulfonamido)isophthalate in 95% (2.715 g) yield as a white solid.

To a stirred suspension of NaH (0.24 g, 10 mmol, 60% in oil dispersion) in 10 mL of DMF was added dimethyl 5-(methylsulfonamido)isophthalate (1.435 g, 5 mmol) followed by iodomethane (0.62 mL, 10 mmol) at room temperature. After 5 h, the reaction was quenched by H₂O (25 mL). Then the reaction mixture was extracted with EtOAc, further washed with H₂O to remove excess of DMF, dried over anhydrous Na₂SO₄ and concentrated. The crude product thus obtained was washed with hexanes to give dimethyl 5-(N-methylmethylsulfonamido)isophthalate as a white solid in 91% (1.37 g) yield.

Dimethyl 5-(N-methylmethylsulfonamido)isophthalate (0.842 g, 2.8 mmol) was dissolved in THF:MeOH (1:1) (8 mL) and H₂O (3 mL). Solid NaOH (0.112 g, 2.8 mmol) was added and stirred at room temperature for 18 h. The reaction mixture was concentrated under reduced pressure. Saturated NaHCO₃ (10 mL) was added to the reaction mixture and extracted with toluene (to remove <10% unreacted starting material). The aqueous solution was acidified with dilute HCl (10%), extracted with EtOAc, and dried over anhydrous Na₂SO₄. The solvent was evaporated and dried under reduced pressure to give 3-(methoxycarbonyl)-5-(N-methylmethylsulfonamido)benzoic acid as a white solid (75%, 0.598 g), which was used without further purification.

### Example 1.3.2

### dimethyl 5-(methylsulfonyloxy)isophthalate

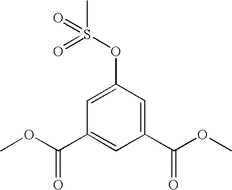

MsCl (0.16 mL, 0.234 g, 2.06 mmol, 1.1 eq) was added to a stirred solution of dimethyl 5-hydroxyisophthalate (0.40 g, 1.86 mmol, 1 eq) and Et₃N (0.78 mL, 0.56 g, 5.6 mmol, 3 eq) in 5 mL anhydrous CH₂Cl₂ at 0° C. under Ar. The reaction was stirred at 0° C. to room temperature over the weekend. The reaction was quenched with water, and the layers were separated. The organic layer was washed with water (×2), brine (×1), and dried over Na₂SO₄. The inorganics were filtered off, and the solvent was removed in vacuo yielding 0.54 g (1.87 mmol, 100% yield) of dimethyl 5-(methylsulfonyloxy)isophthalate.

### Example 1.3.3

### 5-fluoroisophthalic acid

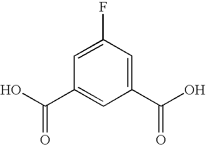

To a gently refluxing solution of 1.9 g (15.3 mmol) of 5-fluoro-m-xylene in about 13.5 mL of pyridine and about 9.5 mL of water was added 13.8 g (87.3 mmol) of KMnO₄ in several portions. The mixture was refluxed for about 7 h, followed by the addition of sodium sulfite to quench the excess KMnO₄. The warm mixture was filtered, and 1N HCl was added to a pH=3. The filtrate was washed with EtOAc, saturated with NaCl, and extracted with the extract of a mixture of (80 mL CHCl₃: 10 mL MeOH: 10 mL H₂O) 3-4 times. The combined extracts were dried over sodium sulfate, filtered, and concentrated to give about 400 mg (14% yield) of 5-fluoroisophthalic acid as a pale yellow solid.

### Example 1.3.4

### 4-fluoro-isophthalic acid

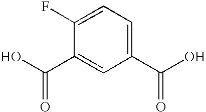

4-fluoro-isophthalic acid was synthesized from 2-fluoro-5-methylbenzoic acid following the procedure described for 5-fluoro-isophthalic acid.

### Example 1.3.5

### dimethyl 5-iodoisophthalate

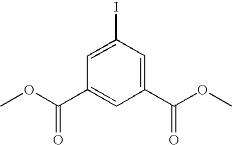

To a stirred solution of dimethyl 5-aminoisophthalate (2.0 g, 9.6 mmol) in 2 N HCl (60 mL) at 0° C. was added NaNO₂ (662 mg, 9.6 mmol) in H₂O (5 mL). The mixture was transferred to a solution of KI (3.2 g, 19.2 mmol) in H₂O (10 mL) at 0° C. The resulting mixture was stirred for 35 min and diluted with EtOAc and H₂O. The layers were separated and the organic layer was washed with 5% Na₂S₂O₃, brine, dried with Na₂SO₄ and concentrated under reduced pressure. The residue was purified by column chromatography (15% EtOAc in hexanes) to provide dimethyl 5-iodoisophthalate (1.53 g, 50%).

### Example 1.3.6

### 3-(methoxycarbonyl)-5-methylbenzoic acid

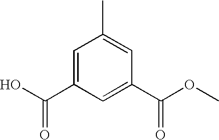

To 5-methylisophthalic acid (Aldrich, 5 g, 27.7) in MeOH (37.5 ml)/THF (112.5 ml), conc. H₂SO₄ (1.25 ml) was added and stirred at 65° C. for 8 h. Reaction mixture was cooled to room temperature and solvent removed. Then reaction mixture was diluted with water and extracted with ethylacetate. Crude residue was column chromatographed to yield 2.5 g of 3-(methoxycarbonyl)-5-methylbenzoic acid as a white solid.

### Example 1.3.7

### 4-methylisophthalic acid

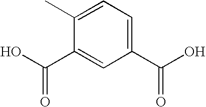

4-methylisophthalic acid was synthesized from 2,5-dimethylbenzoic acid following a similar procedure to that described for 5-fluoroisophthalic acid.

### Example 1.3.8

### dimethyl 5-vinylisophthalate

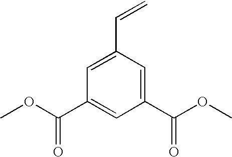

A stirred solution of dimethyl 5-bromoisophthalate (273 mg, 1.0 mmol), potassium vinyltrifluoroborate (134 mg, 1.0 mmol) PdCl₂(dppf).CH₂Cl₂ (16.3 mg, 0.02 mmol) and Et₃N (0.42 mL, 3.0 mmol) in i-PrOH (6 mL) and H₂O (3 mL) was heated to reflux for 3 h. The solution was cooled to room temperature and diluted with EtOAc and H₂O. The layers were separated and the aqueous layer was extracted with EtOAc (2×10 mL). The combined organic layer was washed with brine, dried with Na₂SO₄ and concentrated under reduced pressure. The residue was purified by column chromatography (8% EtOAc in hexanes) to provide dimethyl 5-vinylisophthalate (153.6 mg, 70%).

### Example 1.3.9

### diethyl 5-acetylisophthalate

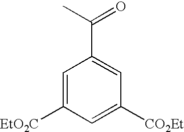

To a stirred solution of diethyl 5-formylisophthalate (2.83 g, 11.3 mmol) in ether (20 mL) was added MeMgBr (3.8 ml of 3.0 M solution, 11.3 mmol) dropwise. The resulting yellow suspension was stirred for 5 h and quenched with saturated aqueous NH₄Cl. The resulting mixture was extracted with EtOAc (2×20 mL). The combined organic layer was washed with brine, dried with Na₂SO₄ and concentrated under reduced pressure. The residue was purified by column chromatography (20% EtOAc in hexanes) to provide diethyl 5-(1-hydroxyethyl)isophthalate (1.15 g, 40%) as a white solid. ¹H NMR (CDCl₃): d 8.60-8.61 (m, 1H), 8.26-8.27 (m, 2H), 5.02-5.09 (m, 1H), 4.45 (q, J=7.2 Hz, 2H), 1.56 (d, J=6.3 Hz, 3H), 1.45 (t, J=7.2, 3H).

A stirred solution of diethyl 5-(1-hydroxyethyl)isophthalate (587 mg, 2.2 mmol) and MnO₂ (960 mg, 11 mmol) was heated to reflux. After 5 h, the reaction was cooled to room temperature and additional MnO₂ (0.6 g) was added and heated to reflux for another 16 h. The reaction mixture was cooled to room temperature and filtered through Celite. The filtrate was concentrated to provide diethyl 5-acetylisophthalate (521 mg, 90%) as a white solid. ¹H NMR (CDCl₃): d 8.62 (s, 1H), 8.56 (s, 2H), 4.32 (q, J=7.2 Hz, 2H), 2.58 (s, 3H), 1.32 (t, J=7.2 Hz, 3H).

### Example 1.3.10

### dimethyl 5-(methylamino)isophthalate

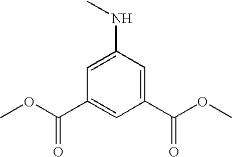

A solution of dimethyl 5-aminoisophthalate (0.250 g, 1.17 mmol, 1 eq) dissolved in 3 mL anhydrous DMF was added dropwise to a stirred suspension of NaH (60% dispersion in mineral oil, 0.14 g, 3.5 mmol, 3 eq) in 2 mL anhydrous DMF at 0° C. under Ar. MeI (0.23 mL, 0.53 g, 3.7 mmol, 3.2 eq) was added dropwise to the resulting mixture. The reaction was stirred at 0° C. to room temperature overnight. The reaction was poured into ice water to quench. The aqueous mixture was extracted with EtOAc (×2), and the combined organic extracts were washed with water (×3), brine (×1), and dried over Na₂SO₄. The inorganics were filtered off, and the solvent was removed in vacuo. Purification via flash chromatography on silica gel yielded 0.14 g (0.63 mmol, 54% yield) of dimethyl 5-(methylamino)isophthalate.

### Example 1.3.11

### dimethyl 5-(dimethylamino)isophthalate

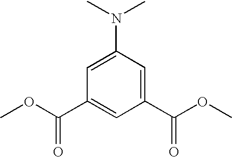

TiCl₄ (1.0 M in CH₂Cl₂, 2.0 mL, 2.0 mmol, 2.1 eq) was add dropwise to a stirred suspension of dimethyl 5-aminoisophthalate (0.2 g, 0.94 mmol, 1 eq) and (HCHO)ₙ (0.059 g, 1.87 mmol, 2.0 eq) in 5 mL anhydrous THF at 0° C. under Ar. After 20 min the ice bath was removed and the mixture was stirred at room temperature for 2 h. The reaction was cooled to 0° C. and NaBH₄ (0.0756 g, 2.0 mmol, 2.1 eq) was added in two approximately equal batches. The reaction was stirred at 0° C. to room temperature over the weekend. The reaction was quenched with water, and the mixture was concentrated in vacuo. The residue was diluted with EtOAc, washed with water (×2), brine (×1), and dried over Na₂SO₄. The inorganics were filtered off. A small amount of silica gel was added, and the solvent was removed in vacuo. The resulting silica gel/crude mixture was loaded onto a column and purified via flash chromatography on silica gel yielded 0.105 g (0.44 mmol, 47% yield) of dimethyl 5-(dimethylamino)isophthalate.

Alternatively, CH₂O (aq, 37%) (3.2 ml, 3.49 g, 43.0 mmol, 6 eq) was added to a stirred solution of the diester (1.5 g, 7.17 mmol, 1 eq) in CH₃CN (50 ml) at 0° C. After 15 min NaBH₃CN (1.09 g, 16.49 mmol, 2.3 eq) was added. The reaction was adjusted to pH ^{˜}7 with HOAc. Stir at 0° C. to RT overnight. The solvent was removed in vacuo, and the residue was partitioned between EtOAc and saturated aqueous NaHCO₃. The layers were separated. The organic layer was washed with water (×3), brine (×1), and dried over Na₂SO₄. The inorganics were filtered off, and the solvent was removed in vacuo. Purification via flash chromatography yielded 1.62 g (6.83 mmol, 95% yield) of dimethyl 5-(dimethylamino)isophthalate.

### Example 1.3.12

### dimethyl 5-(diethylamino)isophthalate

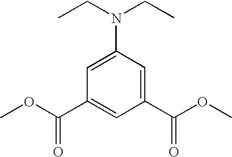

Acetaldehyde (1.07 ml, 0.8 g, 19.12 mmol, 8 eq) was added to a stirred solution of dimethyl 5-aminoisophthalate (0.500 g, 2.39 mmol, 1 eq) in CH₃CN (15 ml) and water (0.5 ml) at 0° C. After 10 min NaBH₃CN (0.395 g, 5.98 mmol, 2.5 eq) was added. The reaction was adjusted to pH ^{˜}7 with HOAc. After 1.5 h the reaction was adjusted to pH ^{˜}7 with HOAc a second time. The reaction was stirred at 0° C. to room temperature overnight. The solvent was removed in vacuo, and the residue was dissolved in EtOAc. The organic layer was washed with saturated aqueous NaHCO₃ (×2), water (×3), brine (×1), and dried over Na₂SO₄. The inorganics were filtered off, and the solvent was removed in vacuo. Purification via flash chromatography yielded 0.575 g (2.17 mmol, 91% yield) of the product.

### Example 1.3.13

### diethyl 5-carbamoylisophthalate

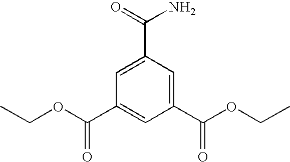

NH₄HCO₃ (0.15 g, 1.89 mmol, 1.26 eq) was added to a stirred solution of 3,5-bis(ethoxycarbonyl)benzoic acid (0.42 g, 1.5 mmol, 1 eq), pyridine (0.24 mL, 0.237 g, 3.0 mmol, 2 eq), and (Boc)₂O (0.45 mL, 0.43 g, 1.95 mmol, 1.3 eq) in 2 mL anh. dioxane under Ar. The reaction was stirred over the weekend to form a white solid. EtOAc was added, but the solid did not dissolve. The mixture was washed with 0.1 N HCl (×2) and water (×4). The solid stayed suspended in the organic layer, but did dissolve. The solvent was removed in vacuo yielding 0.369 g (1.39 mmol, 93% yield) of diethyl 5-carbamoylisophthalate as an insoluble white solid.

### Example 1.3.14

### diethyl 5-(methylcarbamoyl)isophthalate

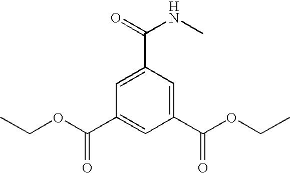

1 drop of Et₃N (catalytic) was added to a stirred solution of 3,5-bis(ethoxycarbonyl)benzoic acid (0.42 g, 1.5 mmol, 1 eq) in SOCl₂ (4 mL, 6.54 g, 55 mmol, 37 eq) under Ar. The solution was heated to reflux at 95° C. After 2 h the reaction was cooled to room temperature, and the solvent was removed in vacuo. The resulting yellow oil was placed under Ar and dissolved in 5 mL anh. CH₂Cl₂. The solution was cooled to 0° C., and MeNH₂ (2.0 M in THF, 2.7 mL, 5.4 mmol, 3.6 eq) was added with stirring. After stirring for 1 h, Et₃N (0.2 mL, 0.15 g, 1.5 mmol, 1 eq) was added. The reaction was stirred at 0° C. to room temperature overnight. The solvent was removed in vacuo. The residue was diluted with saturated NaHCO₃/water and extracted with EtOAc (×3). The combined organics were washed with water (×2), brine (×1), and dried over Na₂SO₄. The inorganics were filtered off, and the solvent was removed in vacuo yielding 0.2914 g (1.0 mmol, 70% yield) of diethyl 5-(methylcarbamoyl)isophthalate.

### Example 1.3.15

### diethyl biphenyl-3,5-dicarboxylate

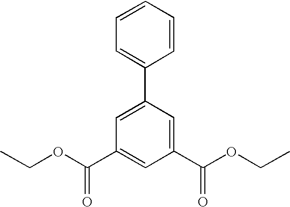

A mixture of Na₂CO₃ (776 mg, 7.32 mmol), Pd (OAc)₂ (4.5 mg, 0.02 mmol), diethyl 5-bromoisophthalate (1 g, 3.66 mmol), phenyl boronic acid (670 mg, 5.49 mmol), distilled water (14 mL) and acetone (12 mL) was stirred at 35° C. for 0.5 h. The reaction solution was then extracted four times with diethyl ether (4×20 mL). The combined organic phase washed with brine, dried over sodium sulfate and then filtered. The solvent was removed under vacuum, and the crude diethyl biphenyl-3,5-dicarboxylate was taken to the next step without any further purification.

### Example 1.3.16

### dimethyl 5-(oxazol-2-yl)isophthalate

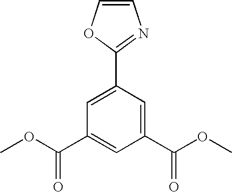

To a stirred solution of oxazole ((0.28 mL, 4.2 mmol) in THF (10 mL) at −78° C. was added nBuLi (2.8 mL 1.6 N solution in hexane, 4.4 mmol). ZnCl₂ (20 mL 0.5M soln, 10 mmol) was added after 30 min and the reaction mixture was warmed up to 0° C. for 1 h. To the resulting mixture was added dimethyl 5-iodoisophthalate (1.28 g, 4.0 mmol) and Pd(PPh₃)₄ and was heated at reflux for 5 h. The reaction mixture was cooled to room temperature and diluted with EtOAc and H₂O. The layers were separated and the organic layer was washed with, brine, dried with Na₂SO₄ and concentrated under reduced pressure. The residue was purified by column chromatography (20% EtOAc in hexanes) to provide dimethyl 5-(oxazol-2-yl)isophthalate (568 mg, 54%).

### Example 1.3.17

### 3-(methoxycarbonyl)-5-(oxazol-5-yl)benzoic acid

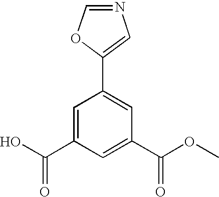

To a stirred solution of diethyl 5-hydroxyisophthalate (4.0 g, 15.9 mmol) in HOAc (40 mL) was added a solution of CAN (19 g, 34.9 mmol) in H₂O (40 mL) dropwise. The reaction mixture was heated at 70° C. for 6 h during which time the color of the solution turned from red to colorless. The reaction mixture was cooled to room temperature and dilute with H₂O and was extracted with EtOAc. The combined organic layer was washed with saturated aqueous NaHCO₃, brine, dried with Na₂SO₄ and concentrated under reduced pressure to provide diethyl 5-formylisophthalate (3.93 g, 99%) as a white solid. ¹H NMR (CDCl₃): d 10.17 (s, 1H), 8.95-8.96 (m, 1H), 8.74-8.75 (m, 2H), 4.50 (q, J=7.2 Hz, 4H), 1.47 (t, J=7.2 Hz, 6H).

To a stirred solution of diethyl 5-formylisophthalate (529 mg, 2.1 mmol) and p-toluenesulfonylmethyl isocyanide (483 mg, 2.5 mmol) in DME (15 mL) and MeOH (15 mL) was added K₂CO₃. The resulting mixture was heated to reflux for 4 h and cooled to room temperature. The solvent was removed and the residue was dissolved in EtOAc and H₂O. The layers were separated and the aqueous layer was extracted with EtOAc (2×20 mL). The combined organic layer was washed with brine, dried with Na₂SO₄ and concentrated under reduced pressure to provide 9 (103 mg, 19%). ¹H NMR (CDCl₃): d 8.63 (s, 1H), 8.49 (s, 2H), 8.00 (s, 1H), 7.54 (s, 1H), 4.00 (s, 6H).

### Example 1.3.18

### dimethyl 5-(pyrrolidin-1-yl)isophthalate

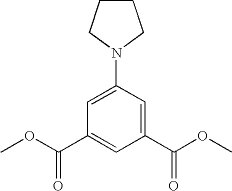

Anhydrous DMF (3 ml) was added to a flask charged with dimethyl 5-aminoisophthalate (0.250 g, 1.2 mmol, 1 eq) and 4-dimethylaminopyridine (0.308 g, 2.52 mmol, 2.1 eq) under Ar. 1,4-diiodobutane (0.16 ml, 0.37 g, 1.20 mmol, 1 eq) was added with stirring and the solution was heated to 90° C. After heating overnight the reaction was not complete. More diiodide (0.25 ml, 1.9 mmol, 1.6 eq) was added and the reaction was heated to 100° C. After heating overnight the reaction was cooled to room temperature and poured in water. The mixture was extracted with EtOAc (×2). The combined organic extracts were washed with water (×3), brine (×1), and dried over Na₂SO₄. The inorganics were filtered off, and the solvent was removed in vacuo. The residue was stirred in CH₂Cl₂ and filtered through cotton to remove any insoluble material. The solvent was removed in vacuo. Purification via flash chromatography yielded the crude product. The crude was triturated with hexanes and the solid was collected via filtration. 0.167 g (0.63 mmol, 53% yield) of the product was collected.

### Example 1.3.19

### dimethyl 5-(piperidin-1-yl)isophthalate

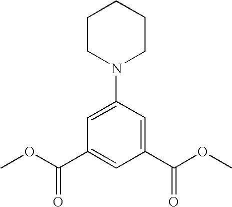

1,5-diiodopentane (0.85 ml, 1.8 g, 5.74 mmol, 3 eq) was added to a stirred solution of dimethyl 5-aminoisophthalate (0.40 g, 1.91 mmol, 1 eq) and DMAP (0.467 g, 3.82 mmol, 2.1 eq) at 100° C. under Ar. After heating overnight the reaction was cooled to room temperature and poured in water. The mixture was extracted with EtOAc (×2). The combined organic extracts were washed with water (×4), brine (×1), and dried over Na₂SO₄. The inorganics were filtered off, and the solvent was removed in vacuo. Purification via flash chromatography yielded 0.1736 g, 0.626 mmol, 33% yield) of the product.

### Example 1.3.20

### dimethyl 5-(4-chlorobutanamido)isophthalate

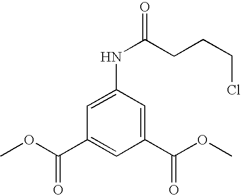

1 drop of Et₃N (catalytic) was added to a stirred solution of 4-chlorobutanoic acid (0.029 ml, 0.35 g, 2.87 mmol 1.2 eq) in SOCl₂ (2 ml, 3.27 g, 27.5 mmol, 11.5 eq) and the mixture was heated to 80° C. After 1.5 h the reaction was cooled to room temperature, and the solvent was removed in vacuo. The flask was evacuated and back-filled with Ar (×3). The residue was dissolved in 2 ml anhydrous CH₂Cl₂. The resulting solution was added dropwise to a stirred suspension of dimethyl 5-aminoisophthalate in 8 ml anhydrous CH₂Cl₂. After 1 h Et₃N (1 ml, 0.73 g, 7.17 mmol, 3 eq) was added. After 2 h the solvent was removed in vacuo, and the resulting residue was dissolved EtOAc. The organic layer was washed with saturated aqueous NaHCO₃ (×2), water (×3), brine (×1), and dried over Na₂SO₄. The inorganics were filtered off, and the solvent was removed in vacuo. Purification via flash chromatography yielded 0.6353 g (2.0 mmol, 85% yield) of the product.

### Example 1.3.21

### dimethyl 5-(2-oxopyrrolidin-1-yl)isophthalate

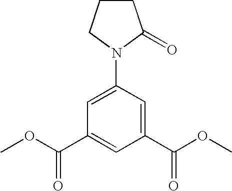

A solution of dimethyl 5-(4-chlorobutanamido)isophthalate (0.635 g, 2.02 mmol, 1 eq) dissolved in 5 ml anhydrous DMF was added dropwise to a stirred suspension of NaH (60% dispersion in oil, 0.101 g, 2.53 mmol, 1.25 eq) in 2 ml anhydrous DMF at 0° C. under Ar. The reaction was stirred at 0° C. to room temperature overnight. After stirring overnight the reaction was heated to 100° C. for 19 h. After cooling to room temperature the reaction was poured into ice-water to quench. The mixture was extracted with EtOAc (×1). The organic layer was washed with water (×4), brine (×1), and dried over Na₂SO₄. The inorganics were filtered off, and the solvent was removed in vacuo. Purification via flash chromatography yielded 0.3487 g (1.26 mmol, 62% yield) of the product.

### Example 1.3.22

### dimethyl 5-(1H-pyrrol-1-yl)isophthalate

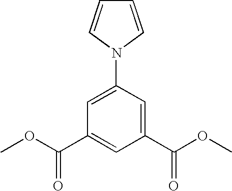

2,5-dimethoxytetrahydrofuran (0.74 ml, 0.76 g, 5.74 mmol, 1.2 eq) was added to a stirred suspension of dimethyl 5-aminoisophthalate (1.0 g, 4.78 mmol, 1 eq) in 7 ml acetic acid under Ar. The mixture was heated to reflux at 135° C. After 45 min the reaction was cooled to RT, and the solvent was removed in vacuo. The residue was stirred in saturated aqueous NaHCO₃/EtOAc overnight. The layers were separated. The organic layer was washed with saturated aqueous NaHCO₃ (×1), water (×2), brine (×1), and dried over Na₂SO₄. The inorganics were filtered off, and the solvent was removed in vacuo. Purification via flash chromatography yielded 0.288 g (1.11 mmol, 23% yield) of the product. A significant amount of crude product was also collected.

### Example 1.3.23

### dimethyl 5-(pyridin-2-yl)isophthalate

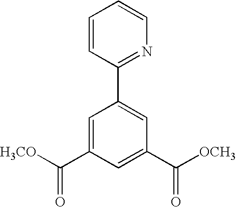

To dimethyl 5-iodoisophthalate (Matrix Scientific, 800 mg, 2.5 mmol) in THF (20 ml), 2-pyridine boronic acid N-phenyldiethanol amine ester (Aldrich, 1.8 g, 6.6 mmol), K₂CO₃ (912 mg, 6.6 mmol), triphenyl phosphine (173 mg, 0.66 mmol) were added followed by Pd(OAc)₂ and cuprous iodide (251 mg, 1.32 mmol). After refluxing for 24 h, reaction mixture was filtered through a pad of celite. Residual solvent was evaporated on a rotavap under reduced pressure and the crude was dissolved in ethyl acetate. Insoluble material was filtered off and the remaining residue was evaporated to dryness and column purified (60% ethylacetate/40% hexanes) to yield 400 mg of dimethyl 5-(pyridin-2-yl)isophthalate as yellow solid.

### Example 1.3.24

### dimethyl 5-(pyridin-3-yl)isophthalate

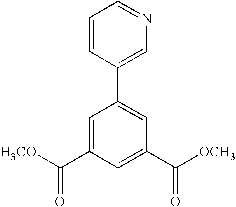

To dimethyl 5-iodoisophthalate (Matrix scientific) in 1,4-dioxane (10 ml), pyridine 3-boronoic acid, sodium carbonate (2M aqueous solution) and Pd(PPh₃)₄ was added and heated at 90° C. for 4 h. Then reaction mixture was diluted with ether, washed with water, brine and dried. Volatiles were removed under vacuum and the crude residue was column chromatographed (60% ethylacetate/40% hexanes) to yield 450 mg of dimethyl 5-(pyridin-3-yl)isophthalate as pale yellow solid.

### Example 1.3.25

### dimethyl 2′-methoxybiphenyl-3,5-dicarboxylate

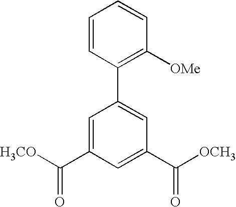

To dimethyl 5-bromoisophthalate (1.5 g, 5.5 mmol, Aldrich) in i-PrOH (33.3 ml) and water (16.7 ml), 2-methoxy phenyl boronic acid (Aldrich), triethyl amine (841 mg, 8.25 mmol) and PdCl₂(dppf) (180 mg, 0.22 mmol) were added and the reaction mixture was refluxed for 4 h. Then reaction mixture was diluted with ether, washed with water, brine and dried. Volatiles were removed under vacuum and the crude residue was column chromatographed (30% ethylacetate/70% hexanes) to yield 650 mg of dimethyl 2′-methoxybiphenyl-3,5-dicarboxylate as white solid.

### Example 1.2.26

### dimethyl 5-(pyrazin-2-yl)isophthalate

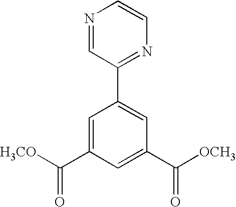

To dimethyl 5-bromoisophthalate (617 mg, 2.26 mmol) in toluene (10 ml), 2-tributylstannyl pyrazine (1 g, 2.71 mmol) was added followed by Pd(PPh₃)₄ (102 mg, 0.09 mmol). Then reaction mixture was refluxed for 22 h. Then the reaction mixture was filtered through celite and volatiles were removed under vacuum. Crude residue was column chromatographed (50% ethylacetate/50% Hexanes) to obtain 455 mg of dimethyl 5-(pyrazin-2-yl)isophthalate as a pale yellow solid.

### Example 1.2.27

### 5-(1H-pyrazol-4-yl)isophthalic acid

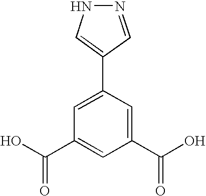

Following standard cross coupling procedure described herein, dimethy 5-bromoisophthalate (623 mg, 2.3 mmol) and 4-pyrazoleboronic acid pinacol ester (443 mg, 2.3 mmol) were reacted. The resulting aqueous layer was acidified to pH 5 and extracted with EtOAc to provide 5-(1H-pyrazol-4-yl)isophthalic acid as a yellow solid.

### Example 1.2.28

### dimethyl 5-(3-hydroxypyrrolidin-1-yl)isophthalate

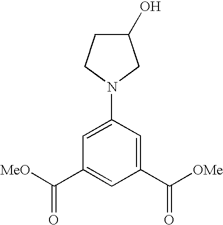

1,4-dibromo-2-butanol (85%, 0.48 ml, 1.1 g, 4.78 mmol, 1 eq) was added to a stirred suspension of K₂CO₃ (1.982 g, 14.34 mmol, 3 eq) in 5 ml triethyl phosphate under Ar. Dimethyl 5-aminoisophthalate (1.00 g, 4.78 mmol, 1 eq) was added and the mixture was heated to reflux at 150° C. After refluxing for 9 h the reaction was cooled to room temperature. The mixture was diluted with Et₂O/H₂O and the layers were separated. The organic layer was washed with water (×3), brine (×1), and dried over Na₂SO₄. The inorganics were filtered off, and the solvent was removed in vacuo. Purification via flash chromatography yielded the crude product. After concentrating in vacuo the residue was cooled to 0° C. Dropwise addition of ice-water to the rapidly stirred solution resulted in the formation of a yellow solid. 0.45 g (1.61 mmol, 34% yield) of dimethyl 5-(3-hydroxypyrrolidin-1-yl)isophthalate was collected via filtration.

### Example 1.2.29

### dimethyl 5-(3-oxopyrrolidin-1-yl)isophthalate

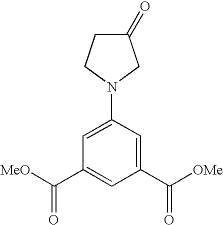

Trifluoroacetic acid (0.061 ml, 0.091 g, 0.794 mmol, 0.5 eq) was added dropwise to a stirred solution of dimethyl 5-(3-hydroxypyrrolidin-1-yl)isophthalate (0.4434 g, 1.59 mmol, 1 eq), anhydrous pyridine (0.135 ml, 0.13 g, 1.67 mmol, 1.05 eq), anhydrous DMSO (0.124 ml, 0.13 g, 1.67 mmol, 1.05 eq), and 1,3-dicyclohexylcarbodiimide (0.655 g, 3.18 mmol, 2 eq) in 5 ml anhydrous benzene at ° C. under Ar. After stirring at 0° C. to room temperature overnight the reaction was diluted with Et₂O/H₂O and stirred for 20 h. The mixture was filtered through cotton and the layers were separated. The organic layer was washed with water (×3), brine (×1), and dried over Na₂SO₄. The inorganics were filtered off and the solvent was removed in vacuo. Purification via flash chromatography yielded only crude product. Purification via a second column yielded 0.201 g (0.73 mmol, 46% yield) of dimethyl 5-(3-oxopyrrolidin-1-yl)isophthalate.

### Example 1.2.30

### dimethyl 5-(3,3-dihydroxypyrrolidin-1-yl)isophthalate

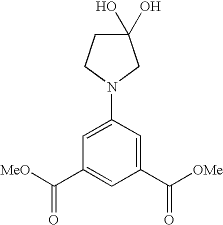

Dimethyl 5-(3-oxopyrrolidin-1-yl)isophthalate (0.1114 g, 0.401 mmol, 1 eq) and NH₄Cl (0.086 g, 1.61 mmol, 4 eq) in 5 ml anhydrous MeOH were heated to reflux at 80° C. for 22 h. After cooling to room temperature the solvent was removed in vacuo. The residue was stirred in EtOH and filtered through cotton to remove any insoluble material. Purification via flash chromatography yielded 0.082 g, (0.25 mmol, 63% yield) of dimethyl 5-(3,3-dihydroxypyrrolidin-1-yl)isophthalate.

### Example 1.2.31

### dimethyl 5-(1H-imidazol-1-yl)isophthalate

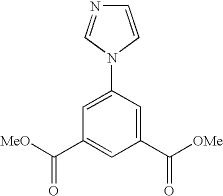

Dimethyl 5-aminoisophthalate (1.00 g, 4.78 mmol, 1 eq) and glyoxal trimer 2H₂O (1.004 g, 4.78 mmol, 1 eq) were stirred in 6 ml EtOH overnight. NH₄Cl (0.5114 g, 9.56 mmol, 2 eq) was added. After 15 min aqueous formaldehyde (37%, 0.71 ml, 0.78 g, 9.56 mmol, 2 eq) was added and the mixture was heated to reflux at 90° C. After 1 h the reaction was cooled to room temperature. After the dropwise addition of H₃PO₄ (85%, 0.65 ml, 1.1 g, 9.56 mmol, 2 eq) the reaction was heated to reflux at 95° C. After 6 h the reaction was cooled to room temperature and the solvent was removed in vacuo. The residue was stirred in CHCl₃ and the mixture was filtered through cotton to remove any insoluble material. Purification via flash chromatography yielded 0.7329 g (2.82 mmol, 59% yield) of dimethyl 5-(1H-imidazol-1-yl)isophthalate.

### Example 1.2.32

### diethyl 5-(1H-imidazol-2-yl)isophthalate

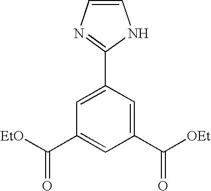

NH₃ (2.0 M in MeOH, 4.8 ml, 9.6 mmol, 8 eq) was added to a flask charged with diethyl 5-formylisophthalate (0.300 g, 1.2 mmol, 1 eq) and glyoxal trimer 2H₂O (0.252 g, 1.2 mmol, 1 eq) at 0° C. under Ar. The reaction was stirred at 0° C. to room temperature overnight. The solvent was removed in vacuo. The residue was stirred in EtOAc and filtered through cotton to remove any insoluble material. Purification via flash chromatography yielded 0.1293 g (0.45 mmol, 37% yield) of diethyl 5-(1H-imidazol-2-yl)isophthalate.

### Example 1.2.33

### diethyl 5-(1-methyl-1H-imidazol-2-yl)isophthalate

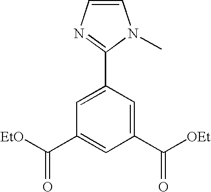

A solution of diethyl 5-(1H-imidazol-2-yl)isophthalate (0.0689 g, 0.239 mmol, 1 eq) in anhydrous THF (2 ml) was added dropwise to a stirred suspension of NaH (60% dispersion in oil, 0.0105 g, 0.263 mmol, 1.1 eq) in anhydrous THF (3 ml) at 0° C. under Ar. After 1 h the reaction was warmed to room temperature. After 1 h the reaction was cooled to 0° C. and MeI (0.016 ml, 0.037 g, 0.263 mmol, 1.1 eq) was added dropwise. The reaction was stirred at 0° C. to room temperature overnight. The reaction was quenched with water and diluted with EtOAc. The organic layer was washed with water (×3), brine (×1), and dried over Na₂SO₄. The inorganics were filtered off and the solvent was removed in vacuo. Purification via flash chromatography yielded 0.305 g (0.1.1 mmol, 42% yield) of diethyl 5-(1-methyl-1H-imidazol-2-yl)isophthalate.

### Example 1.2.34

### dimethyl 2-methoxyisophthalate

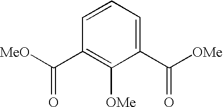

KMNO₄ (19.15 g, 121.2 mmol, 6.6 eq) followed by 2-methoxy-1,3-dimethylbenzene (2.6 ml, 2.5 g, 18.36 mmol, 1 eq) were added to a stirred solution of KOH (3.30 g, 58.74 mmol, 3.2 eq) in 98 ml of water. The reaction was heated to 80° C. After 3 h the reaction was cooled to room temperature. The mixture was filtered through Celite. The solution was adjusted to pH ^{˜}7 with concentrated HCl and again the mixture was filtered through Celite. The solution was adjusted to pH=2-3 with concentrated HCl and extracted with EtOAc (×2). The combined organics were washed with brine (×1) and dried over Na₂SO₄. The inorganics were filtered off and the solvent was removed in vacuo yielding 1.552 g (7.91 mmol, 43% yield) of 2-methoxyisophthalic acid.

SOCl₂ (1.85 ml, 3.03 g, 25.5 mmol, 10 eq) was added dropwise with stirring to a solution of 2-methoxyisophthalic acid (0.500 g, 2.55 mmol, 1 eq) in 10 ml anhydrous MeOH at 0° C. under Ar. The reaction was stirred at 0° C. to room temperature overnight. The solvent was removed in vacuo and the residue dissolved in EtOAc. The solution was washed with saturated aqueous NaHCO₃ (×2), water (×3), brine (×1), and dried over Na₂SO₄. The inorganics were filtered off and the solvent was removed in vacuo yielding 0.6785 g (3.01 mmol, 118% yield) of dimethyl 2-methoxyisophthalate with some impurities.

### Example 1.2.35

### dimethyl 2-(benzyloxy)isophthalate

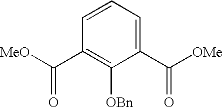

BBr₃ (1.0M in CH₂Cl₂, 7.53 ml, 7.53 mmol, 2.5 eq) was added dropwise to a stirred solution of dimethyl 2-methoxyisophthalate (0.6785 g, 3.01 mmol, 1 eq) in anhydrous CH₂Cl₂ (4 ml) at 0° C. under Ar. After 30 min the reaction was warmed to room temperature. After 2 h the reaction was quenched anhydrous MeOH (1 ml) and stirred overnight. The solvent was removed in vacuo and the residue dissolved in EtOAc. The organic layer was washed with saturated aqueous NaHCO₃ (×2), water (×3), brine (×1), and dried over Na₂SO₄. The inorganics were filtered off and the solvent was removed in vacuo. Purification via flash chromatography yielded 0.4045 g (1.92 mmol, 64% yield) of dimethyl 2-hydroxyisophthalate.

Benzyl bromide (0.34 ml, 0.49 g, 2.89 mmol, 1.5 eq) was added to a stirred suspension of dimethyl 2-hydroxyisophthalate (0.4045 g, 1.92 mmol, 1 eq) and K₂CO₃ (0.5317 g, 3.85 mmol, 2 eq) in anhydrous DMF (2 ml) under Ar. After 48 h the reaction was diluted with Et₂O. The mixture was washed with water (×4), brine (×1), and dried over Na₂SO₄. The inorganics were filtered off and the solvent was removed in vacuo. Purification via flash chromatography yielded 0.5207 g (1.73 mmol, 90% yield) of dimethyl 2-(benzyloxy)isophthalate.

### Example 1.2.36

### dimethyl 4′-(dimethylamino)biphenyl-3,5-dicarboxylate

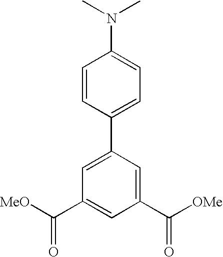

To dimethyl 5-bromoisophthalate (1.38 gm, 5.05 mmol) (Matrix scientific) in 1,4-dioxane (20 ml), 4-(N,N-dimethyl amino phenyl boronic acid) (1.0 g, 6.06 mmol), sodium carbonate (2M aqueous solution) (2.12 g in 10 ml water) and Pd(PPh₃)₄ (589 mg, 0.51 mmol) was added and heated at 90° C. for 4 h. Then reaction mixture was diluted with ether, washed with water, brine and dried. Volatiles were removed under vacuum and the crude residue was column chromatographed (60% ethylacetate/40% hexanes) to yield 420 mg of dimethyl 4′-(dimethylamino)biphenyl-3,5-dicarboxylate.

### Example 1.2.37

### dimethyl 3′-chlorobiphenyl-3,5-dicarboxylate

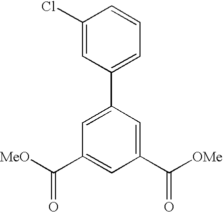

To dimethyl 5-bromoisophthalate (880 mg, 3.22 mmol) (commercial source: Matrix scientific) in 1,4-dioxane (15 ml), 3-chlorophenyl boronic acid) (756 mg, 4.83 mmol), sodium carbonate (2M aqueous solution) (1.38 gms in 6.5 ml water) and Pd(PPh₃)₄ (370 mg, 0.32 mmol) was added and heated at 90° C. for 5 h. Then reaction mixture was diluted with ether, washed with water, brine and dried. Volatiles were removed under vacuum and the crude residue was column chromatographed (10% ethylacetate/90% hexanes) to yield 700 mg of dimethyl 3′-chlorobiphenyl-3,5-dicarboxylate.

### Example 1.2.39

### 3-(methoxycarbonyl)-4-methylbenzoic acid

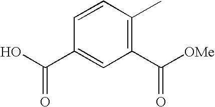

A mixture of 268 mg of the methyl 5-formyl-2-methylbenzoate and 1.08 g (1.76 mmol) of Oxone in 6 mL of DMF was stirred at r.t. for 16.75 h. Water, 1N HCl, and EtOAc were added, and the aqueous layer was extracted with EtOAc. The combined extracts were dried over Na₂SO₄, filtered, and concentrated, and 3-(methoxycarbonyl)-4-methylbenzoic acid, which was used for the next reaction without further purification.

### Example 1.2.40

### 5-(methoxycarbonyl)-2-methylbenzoic acid

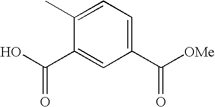

A solution of 305 mg (1.74 mmol) of methyl 3-cyano-4-methylbenzoate and excess Et₃O^{+BF}₄⁻ in 7 mL of CH₂Cl₂ was stirred at 45° C. After 13 h and about 24 h more Et₃O^{+BF}₄⁻ was added and after a further 20 min. the temperature was increased to 50° C. After 37 h, the temperature was increased to 55° C. and heating was continued for 1.5 h. After this time, the crude solution was added with 3 mL of CH₂Cl₂ to 0.16 mL of Et₃SiH in 5 mL of CH₂Cl₂. After the solution was stirred at 55° C. for 2 h, 10 mL of H₂O was added, and the mixture was stirred at 120° C. for 15 min. and the temperature was decreased to 115° C. for 1 h, stopped for 2 h, and then resumed for 17 h. EtOAc was added, and the layers were separated. The organic layer was washed with brine, dried over Na₂SO₄, filtered, and concentrated. Purification by flash silica gel chromatography (7.5% EtOAc/hexanes) provided 25 mg of methyl 3-formyl-4-methylbenzoate as colorless oil with some impurity. 5-(methoxycarbonyl)-2-methylbenzoic acid was synthesized from the aldehyde following the general procedure as described above for the methyl substituted benzoic acid.

### Example 1.4

### Isophthalate/Amine Coupling

### Example 1.4.1

### 3-(methyl((4-methylthiazol-2-yl)methyl)carbamoyl)benzoic acid

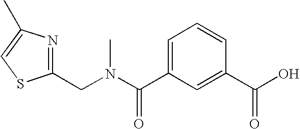

Mono-Methyl isophthalate (0.054 g, 0.30 mmol) was treated with EDCI (0.064 g, 0.33 mmol), HOBt (0.046 g, 0.34 mmol), DIPEA (0.07 mL, 0.4 mmol), and methylthiazole methylamine (0.046 g, 0.36 mmol). The resulting mixture was stirred at room temperature for 15 h under argon followed by quenching with water. The layers were separated and the aqueous layer was extracted with CHCl₃ (2×20 mL). The combined organic layers were dried with Na₂SO₄ and concentrated under reduced pressure. The resulting oil was dissolved in THF (5 mL) to which was added 3 mL of 1.0N LiOH_{(aq)}. The resulting mixture was stirred rapidly for 1.5 h. The volatiles were removed via rotary evaporation and the resulting aqueous solution was extracted with CHCl₃ (×3). The aqueous solution was then acidified to pH 1 with 1N HCl_{(aq)} and extracted with CHCl₃ (×3). The combined organic layers were dried with Na₂SO₄ and concentrated under reduced pressure to provide the corresponding isophthalic acid. This product (0.042 g, 0.11 mmol) was dissolved in DMF and treated with NaH (0.015 g, 0.62 mmol) and MeI (0.04 mL, 0.64 mmol) and stirred overnight. The volatiles were removed via rotary evaporation and the resulting solution was diluted with 1N LiOH and extracted with CHCl₃ (×3). The aqueous solution was then acidified to pH 1 with 1N HCl_{(aq)} and extracted with CHCl₃ (×3). The combined organic layers were dried with Na₂SO₄ and concentrated under reduced pressure to provide N-Methyl-N-(4-methyl-thiazol-2-ylmethyl)-isophthalamic acid. ¹H-NMR: (300 MHz, CDCl₃), d: 8.16 (m, 2H), 7.70 (m, 1H), 7.51 (m, 1H), 6.91 (s, 1H), 5.05 (s, 1.5H), 4.75 (s, 0.5H), 3.2-3.0 (m, 3H), 2.46 (s, 3H).

### Example 1.4.2

### (R)-3-(N-methylmethylsulfonamido)-5-(1-phenylethylcarbamoyl)benzoic acid

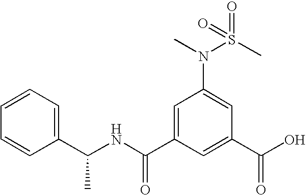

To a stirred solution of 3-(methoxycarbonyl)-5-(N-methylmethan-5-ylsulfonamido)benzoic acid (0.215 g, 0.75 mmol), EDC (0.172 g, 0.9 mmol), HOBt (0.122 g, 0.9 mmol) in DMF/CH₂Cl₂ (1:5 mL) at room temperature was added α-methylbezylamine (0.1 mL, 0.75 mmol) followed by diisopropylethylamine (0.5 mL). The reaction mixture was stirred at room temperature for 16 h. Then water was added and the reaction mixture was extracted with EtOAc. The organic layers were dried over Na₂SO₄ and concentrated. The crude product thus obtained was purified by silica gel flash column chromatography (3% MeOH in CHCl₃) to provide the corresponding amide 10 (0.343 g) which was dissolved in THF:MeOH (1:1) (6 mL) and H₂O (2 mL). Solid NaOH (80 mg, 2.0 mmol) was added and stirred at room temperature for 6 h. The reaction mixture was concentrated under reduced pressure. Saturated NaHCO₃ (10 mL) solution was added to the reaction mixture and extracted with toluene (to remove organic impurities). The aqueous reaction mixture was acidified with diluted HCl (10%), extracted with EtOAc, dried over anhydrous Na₂SO₄. The solvent was evaporated and dried under reduced pressure to give 3-(N-methylmethan-5-ylsulfonamido)-5-((1-phenylethyl)carbomoyl)benzoic acid (0.198 g, 60%) as a white solid.

### Example 1.4.3

### 3-tert-butyl-5-(methyl((4-methylthiazol-2-yl)methyl)carbamoyl)benzoic acid

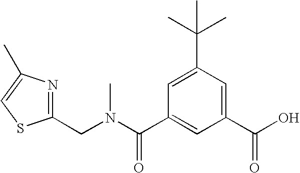

1 drop of Et₃N (catalytic) was added to a stirred solution of 5-tert-butylisophthalic acid (0.145 g, 0.65 mmol, 1 eq) in SOCl₂ (5 mL, 8.18 g, 68.7 mmol, 106 eq) under Ar. The solution was heated to reflux at 95° C. until no solid was visible. The reaction was cooled to room temperature, and the solvent was removed in vacuo. The resulting yellow oil was placed under Ar and dissolved in 4 mL anh. CH₂Cl₂. A solution of N-methyl-1-(4-methylthiazol-2-yl)methanamine (0.100 g, 0.78 mmol, 1.2 eq) in 2 mL anh. CH₂Cl₂ was added with stirring. After stirring for 1 h, Et₃N (0.182 mL, 0.132 g, 1.3 mmol, 2 eq) was added. After stirring for 1 h, the solvent was removed in vacuo. The residue was diluted with saturated NaHCO₃ and extracted with EtOAc (×3). The combined organics were washed with water (×3), brine (×1), and dried over Na₂SO₄. The inorganics were filtered off, and the solvent was removed in vacuo. Purification via flash chromatography eluting with 2-7% MeOH in CHCl₃ yielded 0.085 g (0.256 mmol, 39% yield) of the crude product which was used without further purification.

### Example 1.4.4

### methyl 3-(hydroxymethyl)-5-(methyl((4-methylthiazol-2-yl)methyl)carbamoyl)benzoate

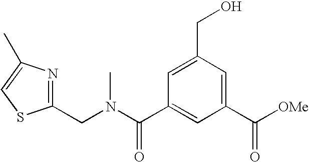

A solution of N-methyl-1-(4-methylthiazol-2-yl)methanamine (511 mg, 3.037 mmol) and 3-(hydroxymethyl)-5-(methoxycarbonyl)benzoic acid (702.5 mg, 3.34 mmol) in DCM (50 mL) were added diisopropylthylamine (3 mL, excess), HOBt (410 mg, 3.34 mmol) and EDCI (754.1 mg, 3.948 mmol). The resulting solution was stirred at room temperature for overnight. The reaction mixture was diluted with chloroform, washed with sodium bicarbonate saturated aqueous solution and separated. The aqueous layer was extracted one more time with chloroform. The combined organic layers were concentrated to give a residue, which was purified with flash chromatography to produce the desired compound (840 mg). ¹H NMR (300 MHz, CDCl₃), d: 8.011 (m, 1.5H), 7.876 (br, 0.5H), 7.683 (m, 1H), 6.749 (m, 1H), 5.579 (m, 0.7H), 5.061 (br, 0.3H), 4.641 (br, 1.2H), 4.525 (br, 0.8H), 3.875 (m, 3H), 3.692 (m, 1H), 3.457 (m, 1H), 2.345 (m, 5H), 2.034 (m, 2H).

### Example 1.5

### Post Isophthalate Coupling Modifications/Amide Alkylation

### Example 1.5.1

### methyl 3-(methyl(4-methylthiazol-2-yl)carbamoyl)benzoate

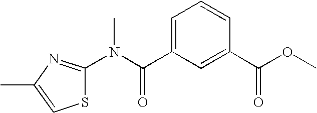

A solution of methyl 3-(4-methylthiazol-2-ylcarbamoyl)benzoate (0.1786 g, 0.65 mmol, 1 eq) dissolved in 1 mL anh. DMF was added dropwise to a stirred suspension of NaH (60% dispersion in mineral oil, 0.052 g, 1.3 mmol, 2 eq) in 2 mL anh. DMF at 0° C. under Ar. MeI (0.132 mL, 0.30 g, 2.13 mmol, 3.3 eq) was added dropwise to the resulting mixture. The reaction was stirred at 0° C. to room temperature overnight. The reaction was poured into ice water to quench. The aqueous mixture was extracted with EtOAc (×3). The combined organic extracts were washed with water (×4), brine (×1), and dried over Na₂SO₄. The inorganics were filtered off, and the solvent was removed in vacuo. Purification via flash chromatography on silica gel yielded 0.14 g (0.48 mmol, 74% yield) of the desired product.

### Example 1.5.2

### methyl 3-(methyl((6-methylpyridin-2-yl)methyl)carbamoyl)benzoate

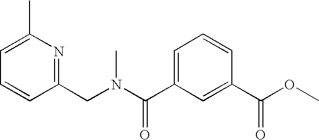

A solution of the crude methyl 3-((6-methylpyridin-2-yl)methylcarbamoyl)benzoate (0.119 g, 0.42 mmol, 1 eq) in 0.5 mL anh. DMF was added dropwise to a stirred suspension of NaH (60% dispersion in mineral oil, 0.025 g, 0.63 mmol, 1.5 eq) in 0.5 mL anh. DMF at 0° C. under Ar. MeI (0.065 mL, 0.15 g, 1.05 mmol, 2.5 eq) was added dropwise to the resulting mixture. The reaction was stirred at 0° C. to room temperature overnight. The reaction was quenched with water and diluted with EtOAc. The layers were separated. The organic layer was washed with water (×3), brine (×1), and dried over Na₂SO₄. The inorganics were filtered off, and the solvent was removed in vacuo. Purification via flash chromatography on silica gel yielded 0.0477 g (0.16 mmol, 38% yield) of the desired product.

### Example 1.5.3

### methyl 3-(2-hydroxyethylcarbamoyl)-5-(methyl((4-methylthiazol-2-yl)methyl)carbamoyl)benzoate

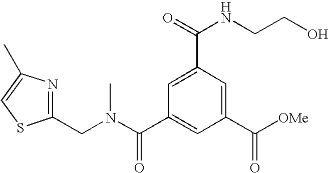

Methyl 3-(2-hydroxyethylcarbamoyl)-5-(methyl((4-methylthiazol-2-yl)methyl)carbamoyl)benzoate was generated from 3-(methoxycarbonyl)-5-(methyl((4-methylthiazol-2-yl)methyl)carbamoyl)benzoic acid by coupling with ethanolamine under standard amide coupling conditions known in the art.

### Example 1.5.4

### methyl 3-(4,5-dihydrooxazol-2-yl)-5-(methyl((4-methylthiazol-2-yl)methyl)carbamoyl)benzoate

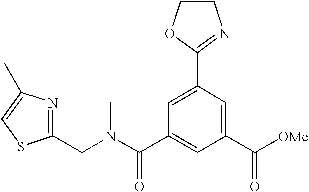

To a stirred solution of methyl 3-(2-hydroxyethylcarbamoyl)-5-(methyl((4-methylthiazol-2-yl)methyl)carbamoyl)benzoate (98 mg, 0.25 mmol) in CH₂Cl₂ (3 mL) was added SOCl₂ (20 μt, 0.28 mmol). After 1 h, the solution was cooled to 0° C. and quenched with H₂O dropwise. The solution was neutralized to pH 7 with 1N NaOH and the layers were separated. The aqueous layer was extracted with CH₂Cl₂ (2×10 mL). The combined organic layer was washed with brine, dried with Na₂SO₄ and concentrated under reduced pressure to provide the desired product (83.7 mg, 86%).

### Example 1.5.5

### methyl 3-(azidomethyl)-5-(methyl((4-methylthiazol-2-yl)methyl)carbamoyl)benzoate

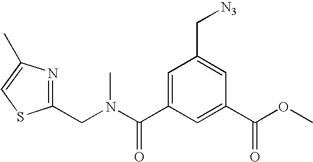

To methyl 3-(hydroxymethyl)-5-(methyl((4-methylthiazol-2-yl)methyl)carbamoyl)benzoate (1.20 g, 3.75 mmol) in benzene and chloroform (5/1, 60 mL) was added SOCl₂ (0.81 mL). The solution was stirred at room temperature for 48 h. The solvent was removed and diluted with CHCl₃, and washed with 0.1 M NaOH and brine. Solvent was removed under reduced pressure to yield chloride as a light yellow syrup (1.2 g). The residue (methyl 3-(chloromethyl)-5-(methyl((4-methylthiazol-2-yl)methyl)carbamoyl)benzoate) was directly used for next reaction without further purification.

The chloride (methyl 3-(chloromethyl)-5-(methyl((4-methylthiazol-2-yl)methyl)carbamoyl)benzoate) (0.60 g, 1.72 mmol) was dissolved in acetone (15 mL) and water (5 mL). NaN₃ (0.67 g, 10.32 mmol) was added, and the solution was refluxed for 16 h. Solvent was removed in vacuo, and the residue was dissolved in CHCl₃. The organic phase was washed with water and brine. Solvent was removed under reduced pressure to yield azide as a pale yellow syrup which was purified with flash chromatography to get desired product (0.5 g): ¹H NMR (300 MHz, CDCl₃), d: 8.116 (br, 2H), 7.683 (s, 1H), 6.949 (s, 1H), 5.030 (br, 1.4H), 4.708 (br, 0.6H), 4.493 (s, 2H), 3.975 (s, 3H), 3.142 (br, 3H), 2.494 (s, 3H).

### Example 1.5.6

### methyl 3-(methoxymethyl)-5-(methyl((4-methylthiazol-2-yl)methyl)carbamoyl)benzoate

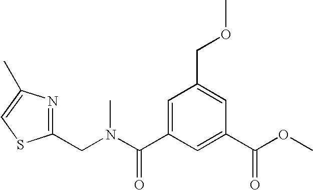

methyl 3-(chloromethyl)-5-(methyl((4-methylthiazol-2-yl)methyl)carbamoyl)benzoate (0.242 g, 0.695 mmol) was dissolved in MeOH (25 mL) at room temperature. NaOMe (0.113 g, 2.084 mmol) was added, and the solution was refluxed for 16 h. Solvent was removed in vacuo, and the residue was dissolved in CHCl₃. The organic phase was washed with water and brine. Solvent was removed under reduced pressure to yield methyl ether as a pale yellow syrup which was purified with column to give the product (0.21 g): ¹H NMR (300 MHz, CDCl₃), d: 8.071 (br, 2H), 7.677 (s, 1H), 6.908 (s, 1H), 4.999 (br, 1.4H), 4.695 (br, 0.6H), 4.518 (s, 2H), 3.921 (s, 3H), 3.415 (br, 3H), 3.048 (s, 3H), 2.451 (s, 3H).

### Example 1.5.7

### methyl 3-formyl-5-(methyl((4-methyloxazol-2-yl)methyl)carbamoyl)benzoate

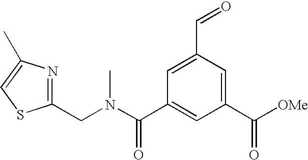

To a solution of methyl 3-(hydroxymethyl)-5-(methyl((4-methyloxazol-2-yl)methyl)carbamoyl)benzoate (293 mg, 0.876 mmol) in DCM (100 mL), Dess-Martin periodinane (446 mg, 1.051 mmol) was added at rt. After 2 hr of stirring, the mixture was poured into a mixture of aqueous 1 M Na₂S₂O₃ (30 mL) and aqueous saturated NaHCO₃ (30 mL), and it was extracted with DCM three times. The combined organic layers were concentrated in vacuum and the residue was purified by flash silica chromatography to give the product (270 mg).

### Example 1.5.8

### methyl 3-(furan-2-yl)-5-(methyl((4-methylthiazol-2-yl)methyl) carbamoyl)benzoate

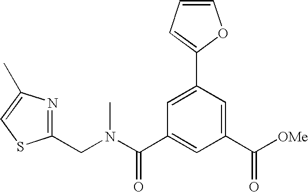

A solution containing methyl 3-iodo-5-(methyl((4-methylthiazol-2-yl)methyl)carbamoyl)benzoate (288 mg, 0.67 mmol) and 2-furanboronic acid (82 mg, 0.74 mmol) and Na₂CO₃ (3.4 mL, 1M aqueous solution) in DMF (15 mL) was degassed under Ar for 10 min. Pd(PPh₃)₄ (80 mg, 0.07 mmol) was added and the mixture was degassed for 2 min. The resulting mixture was heated to 85° C. for 4 h and cooled to room temperature. The mixture was diluted with NH₄Cl and extracted with EtOAc (3×20 mL). The combined organic layer was washed with brine, dried with Na₂SO₄ and concentrated under reduced pressure to provide methyl 3-(furan-2-yl)-5-(methyl((4-methylthiazol-2-yl)methyl) carbamoyl)benzoate (264 mg) as a dark oil which was used for next step without further purification.

### Example 1.5.9

### methyl 3-(methyl((4-methylthiazol-2-yl)methyl)carbamoyl)-5-(1-methyl-1H-pyrrol-2-yl)benzoate

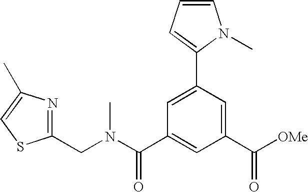

To a stirred solution of methyl 3-iodo-5-(methyl((4-methylthiazol-2-yl)methyl)carbamoyl)benzoate (279 mg, 0.65 mmol) and 1-methyl-2-(tributylstannyl)-1H-pyrrole (0.23 mL, 0.68 mmol) in toluene (10 mL) was added Pd(PPh₃)₄ (80 mg, 0.07 mmol) and the reaction mixture was heated to reflux for 6 h. The resulting mixture was cooled to room temperature and filtered through a pad of Celite. The solvent was removed and the residue was purified by column chromatogaraphy (70% EtOAc in hexanes) to provide methyl 3-(methyl((4-methylthiazol-2-yl)methyl)carbamoyl)-5-(1-methyl-1H-pyrrol-2-yl)benzoate (171 mg) as a yellow oil.

### Example 1.5.10

### methyl 3-(methyl((4-methylthiazol-2-yl)methyl)carbamoyl)-5-(1-methyl-1H-pyrazol-4-yl)benzoate

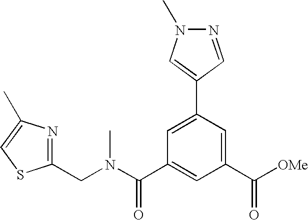

Following standard cross coupling procedure, methyl 3-bromo-5-(methyl((4-methylthiazol-2-yl)methyl)carbamoyl)benzoate (207 mg, 0.54 mmol) and 1-methylpyrazole-4-boronic acid pinacol ester (124 mg, 0.60 mmol) were reacted to provide methyl 3-(methyl((4-methylthiazol-2-yl)methyl)carbamoyl)-5-(1-methyl-1H-pyrazol-4-yl)benzoate (227 mg) as a yellow oil.

### Example 1.5.11

### 3-(6-fluoropyridin-3-yl)-5-(methyl((4-methylthiazol-2-yl)methyl)carbamoyl)benzoic acid

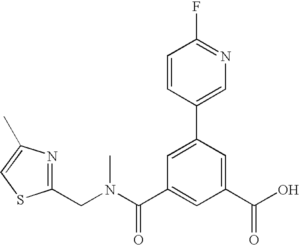

Following standard cross coupling procedure, methyl 3-bromo-5-(methyl((4-methylthiazol-2-yl)methyl)carbamoyl)benzoate (259 mg, 0.68 mmol) and 2-fluoropyridine-5-boronic acid pinacol ester (166 mg, 0.74 mmol) were reacted. The resulting aqueous layer was acidified to pH 2 and extracted with EtOAc. The solvent was removed and the residue was purified by column chromatogaraphy (3% methanol in CHCl₃) to provide 3-(6-fluoropyridin-3-yl)-5-(methyl((4-methylthiazol-2-yl)methyl)carbamoyl)benzoic acid as a solid.

### Example 1.5.12

### methyl 3-(furan-3-yl)-5-(methyl((4-methylthiazol-2-yl)methyl)carbamoyl)benzoate

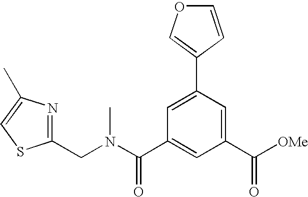

Following standard cross coupling procedure, methyl 3-bromo-5-(methyl((4-methylthiazol-2-yl)methyl)carbamoyl)benzoate (223 mg, 0.58 mmol) and 3-furanboronic acid pinacol ester (125 mg, 0.64 mmol) were reacted to provide methyl 3-(furan-3-yl)-5-(methyl((4-methylthiazol-2-yl)methyl)carbamoyl)benzoate (246 mg) as a yellow oil.

### Example 1.5.13

### 2-hydroxy-3-(methyl((4-methylthiazol-2-yl)methyl)carbamoyl)benzoic acid

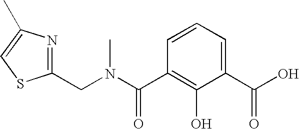

20% Pd(OH)₂/C (0.011 g, 10 wt %) was added to a stirred solution of 2-(benzyloxy)-3-(methyl((4-methylthiazol-2-yl)methyl)carbamoyl)benzoic acid (0.1128 g, 0.284 mmol) in MeOH. The mixture was stirred under a H₂ balloon overnight. Additional 20% Pd(OH)₂/C (0.022 g, 20 wt %) was added. The H₂ balloon was re-inflated and the reaction was stirred overnight. More 20% Pd(OH)₂/C (0.044 g, 40 wt %) was added. The H₂ balloon was re-inflated and the reaction was stirred overnight. The mixture was filtered through Celite and the solvent was removed in vacuo yielding 0.079 g (0.258 mmol, 91% yield) of the product.

### Example 1.5.14

### methyl 3-(fluoromethyl)-5-(methyl((4-methylthiazol-2-yl)methyl)carbamoyl)benzoate

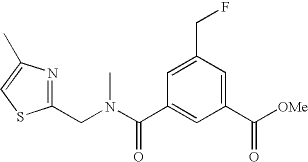

methyl 3-(fluoromethyl)-5-(methyl((4-methylthiazol-2-yl)methyl)carbamoyl)benzoate was synthesized from methyl 3-(hydroxymethyl)-5-(methyl((4-methylthiazol-2-yl)methyl)carbamoyl)benzoate using diethylaminosulfur trifluoride (DAST) following the general monofluoronation procedure described herein. ¹H NMR (300 MHz, CDCl₃), d: 8.211-7.898 (m, 2H), 7.784 (s, 0.7H), 7.420 (br, 0.3H), 6.778 (s, 1H), 5.645-5.076 (m, 3H), 3.897 (m, 3H), 3.768 (m, 1H), 3.519 (m, 1H), 2.363 (m, 5H), 2.188-1.930 (m, 2H).

### Example 1.5.15

### methyl 3-(difluoromethyl)-5-(methyl((4-methylthiazol-2-yl)methyl)carbamoyl)benzoate

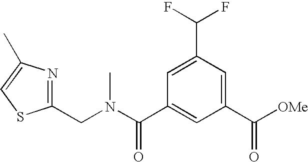

methyl 3-(difluoromethyl)-5-(methyl((4-methylthiazol-2-yl)methyl)carbamoyl)benzoate was synthesized from methyl 3-formyl-5-(methyl((4-methylthiazol-2-yl)methyl)carbamoyl)benzoate using diethylaminosulfur trifluoride (DAST) following the general fluoronation procedure described herein. ¹H NMR (300 MHz, CDCl₃), d: 8.330-8.024 (m, 2H), 7.919 (s, 0.7H), 7.528 (br, 0.3H), 6.902-6.368 (m, 2H), 5.638 (br, 0.7H), 5.048 (br, 0.3H), 3.946-3.746 (m, 4H), 3.509 (m, 1H), 2.412-2.312 (m, 5H), 2.122-1.950 (m, 2H).

### Example 1.5.16

### methyl 3-isopropyl-5-(methyl((4-methylthiazol-2-yl)methyl)carbamoyl)benzoate

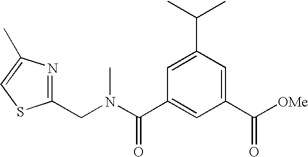

A stirred solution of methyl 3-formyl-5-(methyl((4-methylthiazol-2-yl)methyl)carbamoyl)benzoate (435 mg, 1.309 mmol) in THF at −78° C. was added methyl magnesium bromide (3M in THF, 0.48 mL) slowly. The resulting solution was warmed up to room temperature overnight, quenched with saturated aqueous NH₄Cl solution, extracted with ethyl acetate three times. The combined organic layers were washed with brine, dried with anhydrous Na₂SO₄, concentrated to a residue which was purified by flash column to give the secondary alcohol. The alcohol was not further identified and directly was oxidized with DMP to give the ketone (410 mg). ¹H NMR (300 MHz, CDCl₃), d: 8.680 (s, 1H), 8.368 (m, 2H), 6.947 (s, 1H), 5.031 (br, 1.4H), 4.689 (br, 0.6H), 4.004 (s, 3H), 3.092 (br, 3H), 2.709 (br, 3H), 2.493 (s, 3H).

To a solution of methyltriphenylphosphonium bromide (592.6 mg, 1.661 mmol) in THF at 0° C. was added slowly butyl lithium (1.6 M in hexanes, 1.1 mL). The resulting mixture was further stirred for 45 min, then ketone (410 mg, 1.185 mmol) in THF was added to the reaction mixture. The resulting solution was stirred from 0° C. to room temperature for overnight. The reaction was quenched with saturated aqueous NH₄Cl solution, extracted with ethyl acetate three times. The combined organic layers were washed with brine, dried with anhydrous Na₂SO₄, concentrated to a residue which was purified by flash column to give the olefin. The olefin was not further identified and directly was hydrogenated in methanol with Pd(OH)₂ and hydrogen balloon to give methyl 3-isopropyl-5-(methyl((4-methylthiazol-2-yl)methyl)carbamoyl)benzoate (237 mg). ¹H NMR (300 MHz, CDCl₃), d: 7.985 (s, 2H), 7.565 (s, 1H), 6.917 (s, 1H), 5.002 (s, 1.4H), 4.688 (br, 0.6H), 3.932 (s, 3H), 3.066 (m, 4H), 2.464 (s, 3H), 1.293 (d, J=6 Hz, 6H).

### Example 1.5.17

### methyl 3-(((4-(hydroxymethyl)thiazol-2-yl)methyl)(methyl)carbamoyl)benzoate

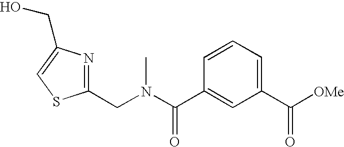

Coupling of the N-methyl-1-(4-((triisopropylsilyloxy)methyl)thiazol-2-yl)methanamine (1.37 g, 4.36 mmol) and 3-(methoxycarbonyl)benzoic acid (863.3 mg, 4.791 mmol) with EDC provided methyl 3-(methyl((4-((triisopropylsilyloxy)methyl)thiazol-2-yl)methyl)carbamoyl)benzoate, which was deprotected with excess aqueous HF (52% in water) in THF at room temperature to produce methyl 3-(((4-(hydroxymethyl)thiazol-2-yl)methyl)(methyl)carbamoyl)benzoate (1.2 g). ¹H NMR (300 MHz, CDCl₃), d: 8.161 (m, 2 H), 7.714 (m, 1H), 7.552 (m, 1H), 7.251 (s, 1H), 5.039 (s, 1.4H), 4.814 (d, J=6.3 Hz, 2H), 4.732 (br, 0.6H), 3.966 (s, 3H), 3.096 (s, 3H).

### Example 1.5.18

### methyl 3-(((4-(fluoromethyl)thiazol-2-yl)methyl)(methyl)carbamoyl)benzoate

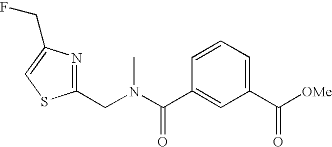

General monofluoronation of methyl 3-(((4-(hydroxymethyl)thiazol-2-yl)methyl)(methyl)carbamoyl)benzoate using the procedure described herein provides methyl 3-(((4-(fluoromethyl)thiazol-2-yl)methyl)(methyl)carbamoyl)benzoate. ¹H NMR (300 MHz, CDCl₃), d: 8.123 (m, 2H), 7.657 (m, 1H), 7.510 (m, 1H), 7.406 (s, 1H), 5.448 (dd, J=3.75, 47.4 Hz, 2H), 5.003 (br, 1.5H), 4.727 (br, 0.5H), 3.916 (s, 3H), 3.070 (s, 3H).

### Example 1.6

### Hydroxylamine Synthesis by Epoxide Ring Opening

### Example 1.6.1

### (2R,3S)-3-amino-4-phenyl-1-(3-(trifluoromethyl)benzylamino)butan-2-ol

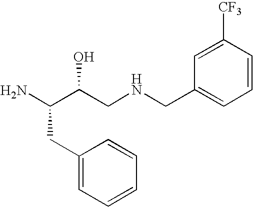

To tert-butyl (S)-1-((S)-oxiran-2-yl)-2-phenylethylcarbamate (Aldrich, 3.0 g, 11.4 mmol) in i-PrOH (50 ml), 3-trifluoromethyl benzyl amine (5 g, 28.5 mmol) was added and the reaction mixture was refluxed for 5 h. Then reaction mixture was cooled to RT and volatiles were removed on a rotavap under reduced pressure. Crude residue was purified by column chromatography to yield 40% of the Boc-amine. Then Boc-amine was dissolved in MeOH (25 ml) and excess 4N HCl in dioxane was added and the reaction mixture was stirred for 16 h at RT. Then volatiles were removed on a rotavap under reduced pressure to yield (2R,3S)-3-amino-4-phenyl-1-(3-(trifluoromethyl)benzylamino)butan-2-ol as the HCl salt in quantitative yield.

### Example 1.6.2

### tert-butyl (2S,3R)-3-hydroxy-4-(3-methoxybenzylamino)-1-phenylbutan-2-ylcarbamate

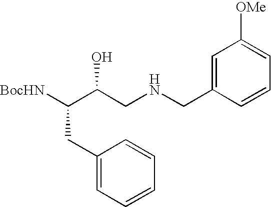

To a stirred solution of tert-Butyl (1-oxiranyl-2-phenylethyl)carbamate (0.5 g, 1.9 mmol) in iPrOH was added 3-methoxybenzyl amine (0.28 mL, 2.1 mmol). The mixture was heated to reflux overnight followed by cooling and removal of the volatiles under reduced pressure. Flash chromatography of the residue resulted in the corresponding aminoalcohol as a solid. ¹H NMR (300 MHz, CDCl₃): d 7.35-7.17 (m, 6H), 6.93-6.78 (m, 3H), 4.65 (d, 1H), 3.90-3.7 (m, 5H), 3.51 (m, 1H), 3.15-2.65 (m, 6H), 1.34 (s, 9H).

### Example 1.6.3

### tert-butyl 4-((1H-benzo[d]imidazol-2-yl)methylamino)-3-hydroxy-1-phenylbutan-2-ylcarbamate

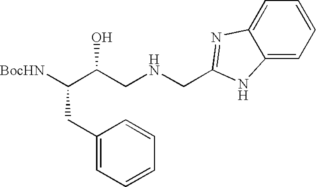

A solution of tert-butyl 1-(oxiran-2-yl)-2-phenylethylcarbamate (185 mg, 0.7 mmol), (1H-benzo[d]imidazol-2-yl)methanamine dihydrochloride salt (232 mg, 1.01 mmol) and Hunig's base (0.49 mL, 2.8 mmol) in iPrOH (6 mL) was refluxed for 12 h. The reaction was cooled to room temperature, solvent evaporated under reduced pressure and chromatographed (5% MeOH/95% CHCl₃) to obtain 175 mg (61%) of the desired product.

### Example 1.6.4

### tert-butyl (2S,3R)-3-hydroxy-1-phenyl-4-((6-(trifluoromethyl)pyridin-3-yl)methylamino)butan-2-ylcarbamate

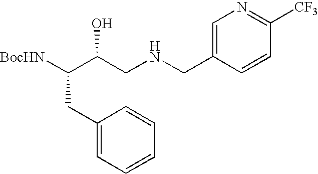

Crude (6-(trifluoromethyl)pyridin-3-yl)methanamine was directly used for opening tert-butyl 1-(oxiran-2-yl)-2-phenylethylcarbamate using the general procedure without further purification. tert-butyl 1-(oxiran-2-yl)-2-phenylethylcarbamate (300 mg, 1.14 mmol) and (6-(trifluoromethyl)pyridin-3-yl)methanamine (300 mg, 1.71 mmol) in isopropanol was heated at 80° C. for 16 h. The solvent was evaporated and purified by silica gel chromatography to afford tert-butyl (2S,3R)-3-hydroxy-1-phenyl-4-((6-(trifluoromethyl)pyridin-3-yl)methylamino)butan-2-ylcarbamate (100 mg) of a light yellow solid: ¹H NMR (300 MHz, CDCl₃+CD₃OD), d: 8.679 (s, 1H), 7.896 (d, J=8.1 Hz, 1H), 7.672 (d, J=7.8 Hz, 1H), 7.344-7.211 (m, 5H), 3.914-3.811 (m, 3H), 3.559 (m, 1H), 3.048-2.986 (m, 1H), 2.901-2.679 (m, 3H), 1.266 (s, 9H).

### Example 1.6.5

### (2R,3S)-3-amino-1-(3-tert-butylbenzylamino)-4-phenylbutan-2-ol

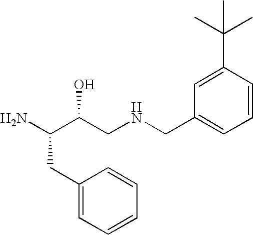

To tert-butyl (S)-1-((S)-oxiran-2-yl)-2-phenylethylcarbamate (Aldrich, 263 mg, 1.0 mmol) in i-PrOH (5 ml), 3-tert-butyl benzyl amine (170 mg, 1.0 mmol) was added and the reaction mixture was refluxed for 5 h. Then reaction mixture was cooled to rt and volatiles were removed on a rotavap under reduced pressure. Crude residue was purified by column chromatography to yield 40% of the Boc-amine. Then Boc-amine was dissolved in MeOH (25 ml) and excess 4N HCl in dioxane was added and the reaction mixture was stirred for 16 h at RT. Then volatiles were removed on a rotavap under reduced pressure to yield (2R,3S)-3-amino-1-(3-tert-butylbenzylamino)-4-phenylbutan-2-ol as HCl salt in quantitative yield.

### Example 1.6.6

### tert-butyl (2S,3R)-4-(3-(diethylamino)benzylamino)-3-hydroxy-1-phenylbutan-2-ylcarbamate

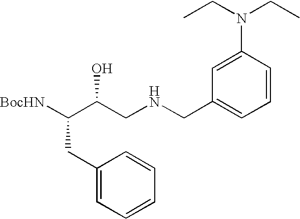

Al(OTf)₃ (0.0168 g, 0.036 mmol, 5 mol %) was added to a flask charged with 3-(aminomethyl)-N,N-diethylaniline (0.19 g, 1.07 mmol, 1.5 eq) under Ar. After stirring for 10 min tert-butyl (S)-1-((S)-oxiran-2-yl)-2-phenylethylcarbamate (0.187 g, 0.71 mmol, 1 eq) was added and the mixture was heated to 70° C. for 1 h. After cooling to room temperature the residue was dissolved in EtOAc with a few drops of water. After 20 min of vigorous stirring the mixture was filtered through cotton and dried over Na₂SO₄. The inorganics were filtered off, and the solvent was removed in vacuo. Purification via flash chromatography yielded 0.0682 g (0.154 mmol, 22% yield) of the product.

### Example 1.6.7

### tert-butyl (2S,3R)-4-(3-(benzylamino)benzylamino)-3-hydroxy-1-phenylbutan-2-ylcarbamate

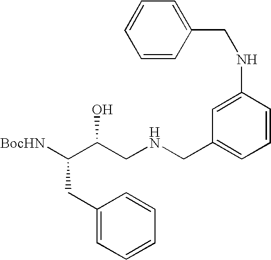

1 ml anhydrous ⁱPrOH was added to a flask charged with 3-(aminomethyl)-N-benzylaniline (0.057 g, 0.269 mmol, 1.3 eq) and tert-butyl (S)-1-((S)-oxiran-2-yl)-2-phenylethylcarbamate (0.054 g, 0.207 mmol, 1 eq) under Ar. The mixture was heated to reflux at 90° C. overnight. After cooling to room temperature the solvent was removed in vacuo. The residue was dissolved in EtOAc, washed with water (×3), brine (×1), and dried over Na₂SO₄. The inorganics were filtered off, and the solvent was removed in vacuo. Purification via flash chromatography yielded 0.0526 g (0.11 mmol, 53% yield) of the product.

### Example 1.6.8

### tert-butyl (2S,3R)-4-(3-(dimethylamino)-5-methoxybenzylamino)-3-hydroxy-1-phenylbutan-2-ylcarbamate

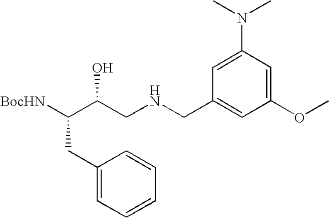

(3-(aminomethyl)-5-(dimethylamino)phenoxy)methylium (0.0361 g, 0.2 mmol, 1.2 eq) was dissolved in the minimum amount of anhydrous CH₂Cl₂ under Ar. tert-butyl (S)-1-((S)-oxiran-2-yl)-2-phenylethylcarbamate (0.0439 g, 0.167 mmol, 1 eq) was added with stirring. Anhydrous CH₂Cl₂ was added dropwise until all of the epoxide had dissolved. The reaction was heated to 50° C. After heating overnight all of the solvent was gone leaving a solid in the flask. Purification via flash chromatography yielded 0.0405 g, (0.091 mmol, 54% yield) of the product.

### Example 1.6.9

### tert-butyl (2S,3R)-4-(3-(dimethylamino)-5-methoxybenzylamino)-3-hydroxy-1-phenylbutan-2-ylcarbamate

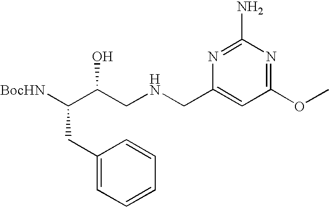

(2-amino-6-(aminomethyl)pyrimidin-4-yloxy)methylium (0.100 g, 0.65 mmol, 1.3 eq) and tert-butyl (S)-1-((S)-oxiran-2-yl)-2-phenylethylcarbamate (0.13 g, 0.5 mmol, 1 eq) were dissolved in anhydrous CH₂Cl₂ (1.2 ml) and anhydrous iPrOH (0.4 ml) under Ar. The reaction was heated to 55° C. After heating overnight all of the solvent was gone leaving a solid in the flask. The residue was dissolved in EtOAc, washed with water (×2), brine (×1), and dried over Na₂SO₄. The inorganics were filtered off, and the solvent was removed in vacuo. Purification via flash chromatography yielded 0.105 g, (0.25 mmol, 50% yield) of the product.

### Example 1.6.10

### tert-butyl (2S,3R)-4-(3-cyanobenzylamino)-3-hydroxy-1-phenylbutan-2-ylcarbamate

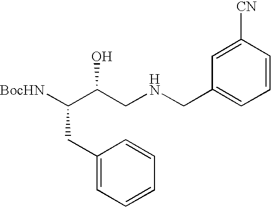

To 3-(aminomethyl)benzonitrile (270 mg, 2.04 mmol), Al(OTf)₃ (47 mg, 0.1 mmol) was added. After 10 min, tert-butyl (S)-1-((S)-oxiran-2-yl)-2-phenylethylcarbamate (268 mg, 1.02 mmol) was added and the reaction mixture was heated to 70° C. for 1.5 h. then the crude residue was loaded onto a column and eluted with Chloroform/MeOH mixture (97:3) to obtain the epoxide opened product in 70% yield.

### Example 1.7

### Alternative Hydroxylamine Synthesis

### Example 1.7.1

### tert-butyl (2S,3R)-4-((5-(2-fluoropropan-2-yl)pyridin-3-yl)methylamino)-3-hydroxy-1-phenylbutan-2-ylcarbamate

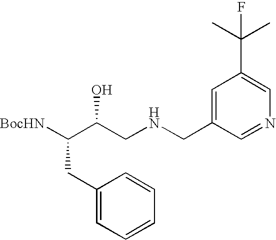

A solution of tert-butyl (S)-1-((S)-oxiran-2-yl)-2-phenylethylcarbamate (1.5 g, 5.7 mmol) in EtOH (35 mL) was added, with stirring, over 1 h to NH₄OH (35 mL) at 0° C. NH₃ gas was bubbled through the reaction mixture during the addition and for 1 h afterward. The reaction mixture was allowed to warm to room temperature and stirred overnight. The resulting slurry was diluted with EtOAc (80 mL), and the organic layer was washed with brine and dried (MgSO₄). Concentration in vacuo, followed by trituration with 10% i-PrOH-EtOAc (overnight stirring), afforded tert-butyl (2S,3R)-4-amino-3-hydroxy-1-phenylbutan-2-ylcarbamate (0.44 g) as a white solid. The mother liquors were concentrated in vacuo and triturated again as above to give an additional quantity of tert-butyl (2S,3R)-4-amino-3-hydroxy-1-phenylbutan-2-ylcarbamate (0.57 g; 64% total yield): ¹H NMR (CD₃OD) d: 1.29 (s, 9H), 2.55 (m, 1H), 2.63 (m, 1H), 2.76 (m, 1H), 3.11 (m, 1H), 3.40 (m, 1H), 3.65 (m, 1 H), 7.10-7.30 (m, 5H).

A solution of tert-butyl (2S,3R)-4-amino-3-hydroxy-1-phenylbutan-2-ylcarbamate (297 mg, 1.06 mmol) in THF was added 5-(2-fluoropropan-2-yl)nicotinaldehyde (180 mg, 1.06 mmol) and stirred for 30 min at room temperature, NaB(OAc)₃H (460 mg, 2.12 mmol) was then added portionwise in 30 min., finally 5 drops of acetic acid was added and the resulting mixture was stirred at the same temperature overnight. The reaction mixture was diluted with EtOAc, and washed with saturated aqueous NaHCO₃. The organic layer was separated and dried (MgSO₄). Concentration in vacuo, followed purification with flash chromatography to give tert-butyl (2S,3R)-4-((5-(2-fluoropropan-2-yl)pyridin-3-yl)methylamino)-3-hydroxy-1-phenylbutan-2-ylcarbamate as a white solid (230 mg, 70% yield). ¹H NMR (CDCl₃): d: 8.776 (d, J=11.5 Hz, 2H), 7.952 (s, 1H), 7.330-7.205 (m, 5H), 4.823 (d, J=7.7 Hz, 1H), 3.900 (s, 2H), 3.842 (m, 1H), 3.586 (m, 1H), 2.995 (m, 1H), 2.856 (m, 1H), 2.762 (m, 2H), 1.363 (s, 9H).

### Example 1.7.2

### methyl 3-(((2R,3S)-3-(tert-butoxycarbonylamino)-2-hydroxy-4-phenylbutylamino)methyl)-5-methoxybenzoate

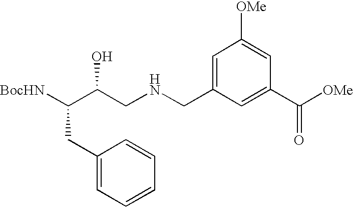

A solution of tert-butyl (2S,3R)-4-amino-3-hydroxy-1-phenylbutan-2-ylcarbamate (300 mg, 1.07 mmol) in THF was added methyl 3-formyl-5-methoxybenzoate (294 mg, ˜80% purity, 1.07 mmol) and stirred for 30 min at room temperature, NaB(OAc)₃H (453.7 mg, 2.14 mmol) was then added portionwise in 30 min, finally 5 drops of acetic acid was added and the resulting mixture was stirred at the same temperature overnight. The reaction mixture was diluted with EtOAc, and washed with saturated aqueous NaHCO₃. The organic layer was separated and dried (MgSO₄). Concentration in vacuo, followed purification with flash chromatography to give the desired product methyl 3-(((2R,3S)-3-(tert-butoxycarbonylamino)-2-hydroxy-4-phenylbutylamino)methyl)-5-methoxybenzoate as a white solid (390 mg, % yield). ¹H NMR (CDCl₃): d: 8.583 (s, 1H), 7.447 (s, 1H), 7.298-7.166 (m, 5H), 7.092 (s, 1H), 3.899 (s, 3H), 3.832 (s, 4H), 3.779 (s, 2H), 3.572 (m, 1H), 2.2.946 (m, 1H), 2.777 (m, 1H), 2.711 (m, 2H), 1.330 (s, 9H).

### Example 1.7.3

### methyl 3-(((2R,3S)-3-(tert-butoxycarbonylamino)-2-hydroxy-4-phenylbutylamino)methyl)benzoate

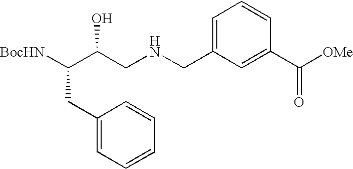

A solution of tert-butyl (2S,3R)-4-amino-3-hydroxy-1-phenylbutan-2-ylcarbamate (200 mg, 0.713 mmol) in THF was added methyl 3-formylbenzoate (117.1 mg, 0.713 mmol) and stirred for 30 min at room temperature. NaB(OAc)₃H (302.5 mg, 1.43 mmol) was then added portionwise in 30 min, finally 5 drops of acetic acid was added and the resulting mixture was stirred at the same temperature overnight. The reaction mixture was diluted with EtOAc, and washed with saturated aqueous NaHCO₃. The organic layer was separated and dried (MgSO₄). Concentration in vacuo was followed by purification with flash chromatography to give the desired product as a white solid (300 mg, % yield). ¹H NMR (CDCl₃): d: 7.999 (m, 2H), 7.589 (m, 1H), 7.451 (m, 1H), 7.254 (m, 5H), 3.956 (s, 3H), 3.892 (m, 3H), 3.549 (m, 1H), 3.040-2.850 (m, 2H), 2.793 (m, 2H), 1.374 (s, 9H).

### Example 1.7.4

### tert-butyl (2S,3R)-4-((5-tert-butylpyridin-3-yl)methylamino)-3-hydroxy-1-phenylbutan-2-ylcarbamate

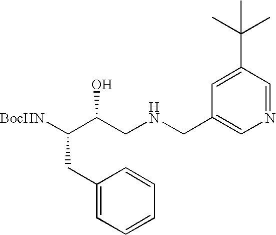

5-tert-butylnicotinaldehyde was coupled with tert-butyl (S)-1-((S)-oxiran-2-yl)-2-phenylethylcarbamate using the typical reductive amination procedure described herein. ¹H NMR (CDCl₃): d: 8.580 (d, J=2.1 Hz, 1H), 8.384 (d, J=1.8 Hz, 1H), 7.667 (t, J=2.1 Hz, 1H), 7.233 (m, 5H), 4.843 (br, 1H), 3.806 (s, 3H), 3.585 (m, 1H), 3.008-2.828 (m, 2H), 2.778 (d, J=5.1 Hz, 2H), 1.372 (s, 9H).

### Example 1.7.5

### tert-butyl (2S,3R)-1-(3,5-difluorophenyl)-3-hydroxy-4-((5-isopropylpyridin-3-yl)methylamino)butan-2-ylcarbamate

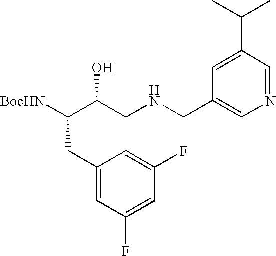

tert-butyl (2S,3R)-1-(3,5-difluorophenyl)-3-hydroxy-4-((5-isopropylpyridin-3-yl)methylamino)butan-2-ylcarbamate was generated using the general procedure described herein starting from tert-butyl (S)-2-(3,5-difluorophenyl)-1-((S)-oxiran-2-yl)ethylcarbamate (Peptech Corp.) in 35% chemical yield. ¹H NMR (CDCl₃): d: 8.393 (m, 2H), 7.525 (m, 1H), 6.768 (m, 2H), 6.664 (m, 1H), 3.804 (s, 3H), 3.574 (m, 1H), 2.977 (m, 2H), 2.778 (m, 3 H), 1.375 (s, 9H), 1.302 (d, J=6.9 Hz, 6H).

### Example 1.7.6

### tert-butyl (2S,3R)-4-((5-(1,1-difluoroethyl)pyridin-3-yl)methylamino)-3-hydroxy-1-phenylbutan-2-ylcarbamate

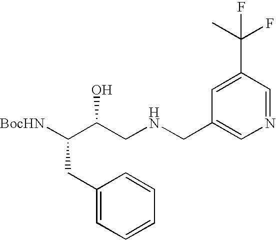

5-(1,1-difluoroethyl)nicotinaldehyde was coupled to tert-butyl (S)-1-((S)-oxiran-2-yl)-2-phenylethylcarbamate using standard reductive amination procedures described herein to generate the desired tert-butyl (2S,3R)-4-((5-(1,1-difluoroethyl)pyridin-3-yl)methylamino)-3-hydroxy-1-phenylbutan-2-ylcarbamate. ¹H NMR (CDCl₃): d: 8.514 (m, 2H), 7.717 (s, 1H), 7.321-7.203 (m, 5H), 3.831 (s, 3H), 3.580 (m, 1H), 2.978 (m, 1H), 2.890-2.755 (m, 3H), 1.724 (d, J=11.1 Hz, 6H), 1.364 (s, 9H).

### Example 1.7.7

### tert-butyl (2S,3R)-4-(3-(1,1-difluoroethyl)benzylamino)-3-hydroxy-1-phenylbutan-2-ylcarbamate

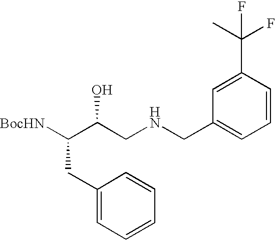

3-(1,1-difluoroethyl)benzaldehyde was coupled to tert-butyl (S)-1-((S)-oxiran-2-yl)-2-phenylethylcarbamate using standard reductive amination procedures described herein to generate the desired tert-butyl (2S,3R)-4-(3-(1,1-difluoroethyl)benzylamino)-3-hydroxy-1-phenylbutan-2-ylcarbamate.

### Example 1.7.8

### (2R,3S)-3-amino-1-((5-chloropyridin-3-yl)methylamino)-4-phenylbutan-2-ol

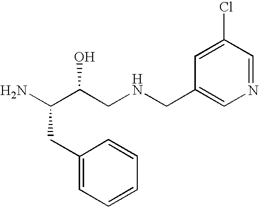

To tert-butyl (2S,3R)-4-amino-3-hydroxy-1-phenylbutan-2-ylcarbamate (672 mg, 2.4 mmol) in THF (15 ml) at RT, 5-chloronicotinaldehyde (Frontier Scientific, 300 mg, 2.4 mmol) was added followed by acetic acid (165 μl). After stirring for 3 h at RT, Na(OAc)₃BH (1.02 g, 4.8 mmol) was added. After stirring for 2 h another 500 mg of Na(OAc)₃BH was added and the reaction mixture was stirred at RT for 48 h. Then AcOH (0.1 ml) was added and stirred for 0.5 h, then saturated aqueous sodium bicarbonate solution was added and stirred for 1 h. Then reaction mixture was extracted with ethyl acetate. Organic layer was dried over sodium sulfate, and volatiles removed under vacuum. Crude residue was column chromatographed to yield the Boc protected amine in 65% yield. The so obtained Boc-protected amine was stirred with 4N HCl in dioxane (4 ml) overnight. Removal of the volatiles yielded (2R,3S)-3-amino-1-((5-chloropyridin-3-yl)methylamino)-4-phenylbutan-2-ol as HCl salt.

### Example 1.7.9

### (2R,3S)-3-amino-1-((5-fluoropyridin-3-yl)methylamino)-4-phenylbutan-2-ol

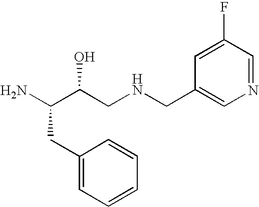

5-fluoronicotinaldehyde (Frontier Scientific) was coupled to tert-butyl (S)-1-((S)-oxiran-2-yl)-2-phenylethylcarbamate using standard reductive amination procedures described herein to generate the desired (2R,3S)-3-amino-1-((5-fluoropyridin-3-yl)methylamino)-4-phenylbutan-2-ol.

### Example 1.7.10

### (2R,3S)-3-amino-1-(3,5-dichlorobenzylamino)-4-phenylbutan-2-ol

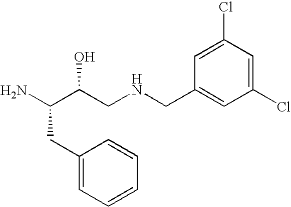

3,5-dichlorobenzaldehyde (Aldrich) was coupled to tert-butyl (S)-1-((S)-oxiran-2-yl)-2-phenylethylcarbamate using standard reductive amination procedures described herein to generate the desired (2R,3S)-3-amino-1-(3,5-dichlorobenzylamino)-4-phenylbutan-2-ol.

### Example 1.7.11

### tert-butyl (2S,3R)-3-hydroxy-4-(3-(2-hydroxypropan-2-yl)benzylamino)-1-phenylbutan-2-ylcarbamate

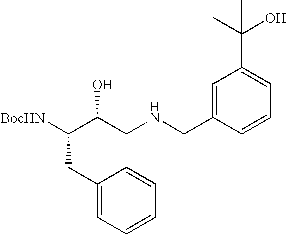

A solution of tert-butyl (2S,3R)-4-amino-3-hydroxy-1-phenylbutan-2-ylcarbamate (230 mg, 0.82 mmol) in THF was added 3-(2-hydroxypropan-2-yl)benzaldehyde (180 mg, 01.06 mmol) and stirred for 30 min at room temperature, NaB(OAc)₃H (302.5 mg, 1.43 mmol) was then added portionwise in 30 min, and the resulting mixture was stirred at the same temperature overnight. The reaction mixture was diluted with EtOAc, and washed with saturated aqueous NaHCO₃. The organic layer was separated and dried (Na₂SO₄). Concentration in vacuo, followed purification with flash chromatography to give the desired product as a white solid (230 mg, 65% yield). ¹H NMR (CDCl₃): d: 8.514 (m, 2H), 7.717 (s, 1H), 7.321-7.203 (m, 5H), 3.831 (s, 3H), 3.580 (m, 1H), 2.978 (m, 1H), 2.890-2.755 (m, 3 H), 1.724 (d, J=11.1 Hz, 6H), 1.364 (s, 9H).

### Example 1.7.12

### N-((2R,3S)-3-amino-2-hydroxy-4-phenylbutyl)-5-(prop-1-en-2-yl)nicotinamide

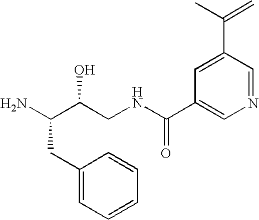

To the 5-(prop-1-en-2-yl)nicotinaldehyde (500 mg, 3.40 mmol) in t-BuOH:water (10:1) (10 ml) at 0° C., 2-methyl-2-butene (9 ml), NaH₂ PO₄ (1.57 g, 13.09 mmol) were added, followed by sodium chlorite (1.53 g, 17.0 mmol) in water. After 1 h, reaction mixture was quenched by the addition of concentrated HCl. Then reaction mixture was stirred for 1 h. Then reaction mixture was basified and extracted with ethyl acetate. Aqueous layer was then acidified and extracted with a 10% MeOH in ethyl acetate and the organic layer was dried and evaporated. Crude residue contained 5-(prop-1-en-2-yl)nicotinic acid which was carried to the next step without any further purification.

To 5-(prop-1-en-2-yl)nicotinic acid (200 mg, 1.23 mmol) in dichlormethane (10 ml) at rt, EDCI (330 mg, 1.72 mmol) and HOBT (200 mg, 1.48 mmol) were added. After stirring at rt for 10 minutes, tert-butyl (2S,3R)-4-amino-3-hydroxy-1-phenylbutan-2-ylcarbamate (344 mg, 1.23 mmol) was added followed by DIPEA (0.2 ml). After stirring overnight at rt, reaction mixture was worked up as usual and residue was column purified (90% ethylacetate/10% hexanes) to yield 280 mg of tert-butyl (2S,3R)-3-hydroxy-1-phenyl-4-(5-(prop-1-en-2-yl)nicotinamido)butan-2-ylcarbamate, which on stirring with 4N HCl in dioxane for 4 h yields the HCl salt of N-((2R,3S)-3-amino-2-hydroxy-4-phenylbutyl)-5-(prop-1-en-2-yl)nicotinamide.

### Example 1.8

### Hydroxylamine Modifications

### Example 1.8.1

### 3-(((2R,3S)-3-amino-2-hydroxy-4-phenylbutylamino)methyl)-5-isopropylphenol

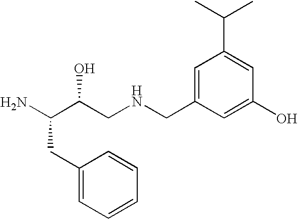

A mixture of 21.2 mg (0.04 mmol) of tert-butyl (2S,3R)-4-(3-(benzyloxy)-5-isopropylbenzylamino)-3-hydroxy-1-phenylbutan-2-ylcarbamate and 7.7 mg of 10% Pd/C in 4 mL of 1.25M HCl in MeOH was stirred at r.t. under H₂ balloon for 20.5 h. The mixture was filtered through Celite, concentrated, and reconcentrated with toluene 3 times. The amine HCl salt was used for the next reaction without further purification.

### Example 1.8.2

### 3-(((2R,3S)-3-amino-2-hydroxy-4-phenylbutylamino)methyl)-5-(prop-1-en-2-yl)phenol

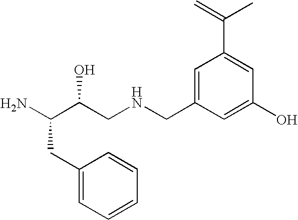

A mixture of 81.8 mg (0.148 mmol) of tert-butyl (2S,3R)-4-(3-(benzyloxy)-5-(2-chloropropan-2-yl)benzylamino)-3-hydroxy-1-phenylbutan-2-ylcarbamate and 16.1 mg of 20% Pd(OH)₂ in 5 mL of MeOH was stirred at r.t. under H₂ balloon for 21 h. The mixture was filtered through Celite and concentrated. The tert-butyl (2S,3R)-4-(3-(2-chloropropan-2-yl)-5-hydroxybenzylamino)-3-hydroxy-1-phenylbutan-2-ylcarbamate product was used in the next reaction without further purification.

A solution of 27.5 mg of tert-butyl (2S,3R)-4-(3-(2-chloropropan-2-yl)-5-hydroxybenzylamino)-3-hydroxy-1-phenylbutan-2-ylcarbamate and 0.8 mL of trifluoroacetic acid in 2 mL of CH₂Cl₂ was stirred at r.t. for 1 h and then concentrated. The 3-(((2R,3S)-3-amino-2-hydroxy-4-phenylbutylamino)methyl)-5-(prop-1-en-2-yl)phenol amine salt was used in the next reaction without further purification.

### Example 1.8.3

### Boc-protected tert-butyl (2R,3S)-3-amino-2-hydroxy-4-phenylbutyl(3-isopropyl-5-(N-methylmethylsulfonamido)benzyl)carbamate

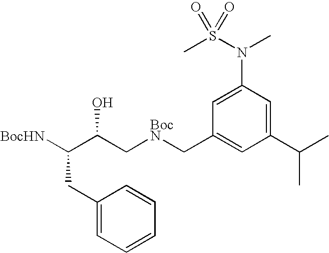

A mixture of 16.4 mg of Boc-protected tert-butyl (2R,3S)-3-amino-2-hydroxy-4-phenylbutyl(3-(N-methylmethylsulfonamido)-5-(prop-1-en-2-yl)benzyl)carbamate and 2.5 mg of 10% Pd/C in 3 mL of MeOH and 1 mL of EtOAc was stirred at r.t. under H₂ balloon for 12.5 h. The mixture was filtered through Celite and concentrated. Purification by flash silica gel chromatography (60% EtOAc/hexanes) provided 13.5 mg of Boc-protected tert-butyl (2R,3S)-3-amino-2-hydroxy-4-phenylbutyl(3-isopropyl-5-(N-methylmethylsulfonamido)benzyl)carbamate in 82% yield.

### Example 1.8.4

### Boc-protected tert-butyl (2R,3S)-3-amino-2-hydroxy-4-phenylbutyl(3-isopropyl-5-(methylsulfonyl)benzyl)carbamate

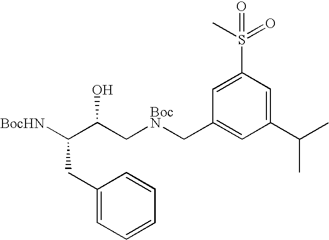

Boc-protected tert-butyl (2R,3S)-3-amino-2-hydroxy-4-phenylbutyl(3-isopropyl-5-(methylsulfonyl)benzyl)carbamate was synthesized in a similar manner to Boc-protected tert-butyl (2R,3S)-3-amino-2-hydroxy-4-phenylbutyl(3-isopropyl-5-(N-methylmethylsulfonamido)benzyl)carbamate by reducing Boc-protected tert-butyl (2R,3S)-3-amino-2-hydroxy-4-phenylbutyl(3-(methylsulfonyl)-5-(prop-1-en-2-yl)benzyl)carbamate.

### Example 1.8.5

### tert-butyl (2S,3R)-4-(3-acetamido-5-isopropylbenzylamino)-3-hydroxy-1-phenylbutan-2-ylcarbamate

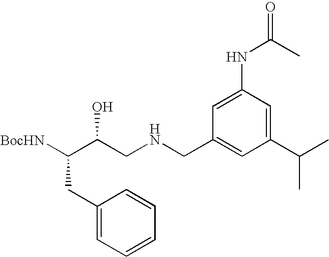

tert-butyl (2S,3R)-4-(3-acetamido-5-isopropylbenzylamino)-3-hydroxy-1-phenylbutan-2-ylcarbamate was synthesized in a similar manner to Boc-protected tert-butyl (2R,3S)-3-amino-2-hydroxy-4-phenylbutyl(3-isopropyl-5-(N-methylmethylsulfonamido)benzyl)carbamate by reducing tert-butyl (2S,3R)-4-(3-acetamido-5-(prop-1-en-2-yl)benzylamino)-3-hydroxy-1-phenylbutan-2-ylcarbamate.

### Example 1.8.6

### 3-(((2R,3S)-3-amino-2-hydroxy-4-phenylbutylamino)methyl)-5-(prop-1-en-2-yl)phenyl dimethylcarbamate

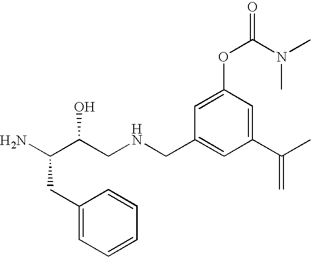

A solution of Boc protected 3-(((2R,3S)-3-amino-2-hydroxy-4-phenylbutylamino)methyl)-5-(prop-1-en-2-yl)phenyl dimethylcarbamate in 3 mL of 1.25 M HCl in MeOH was stirred at r.t. for about 13.5 h. The solution was concentrated, and the crude 3-(((2R,3S)-3-amino-2-hydroxy-4-phenylbutylamino)methyl)-5-(prop-1-en-2-yl)phenyl dimethylcarbamate product was used in the next reaction without further purification.

### Example 1.9

### Hydroxylamine/Isophthalate Coupling

### Example 1.9.1

### N1-((2S,3R)-3-hydroxy-4-(3-methoxybenzylamino)-1-phenylbutan-2-yl)-N3-methyl-N3-((4-methylthiazol-2-yl)methyl)isophthalamide

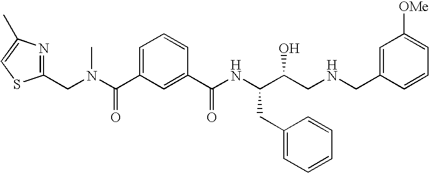

To a stirred solution of tert-butyl 3-hydroxy-4-(3-methoxybenzylamino)-1-phenylbutan-2-ylcarbamate (0.032 g, 0.079 mmol) in dichloromethane was added TFA. The resulting mixture was stirred for 1 hour followed by removal of the volatiles under reduced pressure. This amine was dissolved in dichloromethane, treated with DIPEA, and added to a solution of N-Methyl-N-(4-methyl-thiazol-2-ylmethyl)-isophthalamic acid (0.021 g, 0.071 mmol), EDCI (0.015 g, 0.078 mmol), and HOBt (0.011 g, 0.081 mmol) in dichloromethane. The resulting mixture was stirred at room temperature overnight followed by washing with water, drying with Na₂SO₄, and removal of volatiles under reduced pressure. Flash chromatography of the residue provided the target molecule. ¹H NMR (300 MHz, CDCl₃): d 7.8-7.1 (m, 11H), 6.94-6.76 (m, 4H), 4.95 (s, 1.5H), 4.63 (s, 0.5H), 4.38 (m, 1H), 3.90-3.64 (m, 5H), 3.18-2.74 (m, 9H), 2.44 (s, 3H).

### Example 1.9.2

### N1-((2S,3R)-3-hydroxy-4-(3-methoxybenzylamino)-1-phenylbutan-2-yl)-5-(N-methylmethylsulfonamido)-N3-((R)-1-phenylethyl)isophthalamide

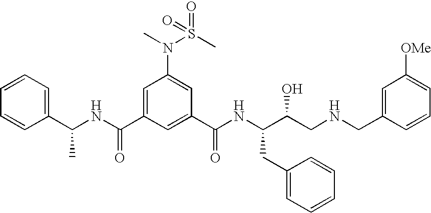

To a solution of tert-butyl 3-hydroxy-4-(3-methoxybenzylamino)-1-phenylbutan-2-ylcarbamate (40 mg, 0.1 mmol) in CH₂Cl₂ (1 mL) was added TFA (0.5 mL) at room temperature. The reaction mixture was stirred at room temperature for 30 min. After this period, the mixture was concentrated under reduced pressure and dissolved in CH₂Cl₂ (1 mL) and stirred with diisopropylethylamine (0.2 mL). This mixture was added to a stirred solution of 3-(N-methylmethan-5-ylsulfonamido)-5-((1-phenylethyl)carbomoyl)benzoic acid (37.6 mg, 0.1 mmol), EDC (24 mg, 0.125 mmol), HOBt (16.9 mg, 0.125 mmol) in DMF/CH₂Cl₂ (1:2 mL). The reaction mixture was stirred at room temperature for 19 h. Then water was added and the reaction mixture was extracted with EtOAc. The organic layers were dried over Na₂SO₄ and concentrated. The crude product N¹-(4-(3-methoxybenzylamino)-3-hydroxy-1-phenylbutan-2-yl)-5-(N-meth-ylmethan-5-ylsulfonamido)-N³-(1-pheynylethyl)isophthalamide thus obtained was purified by silica gel flash column chromatography (10% MeOH in CHCl₃) to provide the target molecule (22.3 mg, 34%) as white solid. ¹H NMR (500 MHz, CDCl₃): δ 1.58 (3H, d), 2.72 (3H, s), 2.74-2.83 (4H, m), 3.22 (3H, s), 3.69-3.83 (6H, m), 4.29-4.33 (1H, m), 5.26 (1H, q), 6.78 (1H, dd), 6.85-6.86 (2H, m), 7.13-7.37 (12H, m), 7.76 (1H, s), 7.89 (2H, s), 8.11 (1H, s).

### Example 1.9.3

### methyl 3-(((2R,3S)-2-hydroxy-3-(3-(methyl((4-methylthiazol-2-yl)methyl)carbamoyl)benzamido)-4-phenylbutylamino)methyl)-5-methoxybenzoate

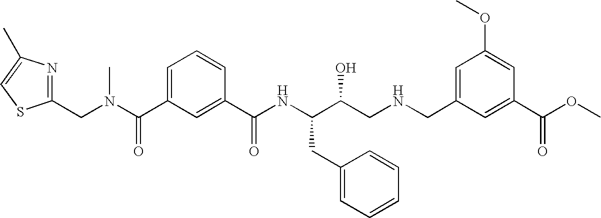

Methyl 3-(((2R,3S)-3-(tert-butoxycarbonylamino)-2-hydroxy-4-phenylbutylamino)methyl)-5-methoxybenzoate (238 mg, 0.519 mmol) in DCM (20 mL) was added TFA (4 mL) at room temperature and stirred for 45 min. The solvent was removed in vacuo and dissolved in DCM, then triethylamine (2 mL, excess), 3-(methyl((4-methylthiazol-2-yl)methyl)carbamoyl)benzoic acid (165.7 mg, 0.571 mmol), EDC (109 mg, 0.571 mmol) and HOBt (77 mg, 0.571 mmol) were added successively to the reaction. The resulting solution was stirred at room temperature fro overnight. The reaction was quenched with diluted aqueous NaHCO₃, separated, extracted with chloroform. The combined organic solvent was removed in vacuo and purified by silica gel chromatography to afford final compound. ¹H NMR (CDCl₃): d: 8.776 (d, J=11.5 Hz, 2H), 7.952 (s, 1H), 7.330-7.205 (m, 5H), 4.823 (d, J=7.7 Hz, 1H), 3.900 (s, 2H), 3.842 (m, 1H), 3.586 (m, 1H), 2.995 (m, 1 H), 2.856 (m, 1H), 2.762 (m, 2H), 1.363 (s, 9H).

### Example 1.9.4

### methyl 3-(((2R,3S)-2-hydroxy-3-(3-(N-methylmethylsulfonamido)-5-((R)-1-phenylethylcarbamoyl)benzamido)-4-phenylbutylamino)methyl)benzoate

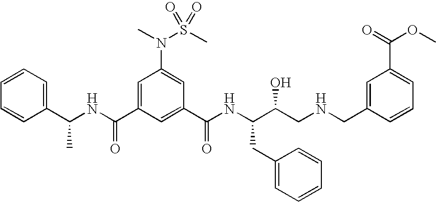

To methyl 3-(((2R,3S)-3-(tert-butoxycarbonylamino)-2-hydroxy-4-phenylbutylamino)methyl)benzoate (220 mg, 0.513 mmol) in DCM (20 mL) was added TFA (4 mL) at room temperature and stirred for 45 min. The solvent was removed in vacuo and dissolved in DCM. DIPA (1.5 mL, excess), (R)-3-(N-methylmethylsulfonamido)-5-(1-phenylethylcarbamoyl)benzoic acid (212.5 mg, 0.565 mmol), EDC (118 mg, 0.616 mmol) and HOBt (83.2 mg, 0.616 mmol) were added successively to the reaction. The resulting solution was stirred at room temperature overnight. The reaction was quenched with diluted aqueous NaHCO₃, separated, extracted with chloroform. The combined organic solvent was removed in vacuo and purified by silica gel chromatography to afford final compound. ¹H NMR (300 MHz, CDCl₃+CD₃OD), d: 8.067-7.769 (m, 5H), 7.533-7.169 (m, 12H), 5.283 (m, 1H), 4.366 (m 1H), 3.899 (s, 3H), 3.899-3.726 (m, 3H), 3.237 (s, 3H), 2.943-2.770 (m, 4H), 2.770 (s, 3H), 1.560 (d, J=6.9 Hz, 3H).

### Example 1.10

### Alternative Coupling

### Example 1.10.1

### 3-(((2R,3S)-2-hydroxy-3-(3-(methyl((4-methylthiazol-2-yl)methyl)carbamoyl)benzamido)-4-phenylbutylamino)methyl)-5-isopropylphenyl morpholine-4-carboxylate

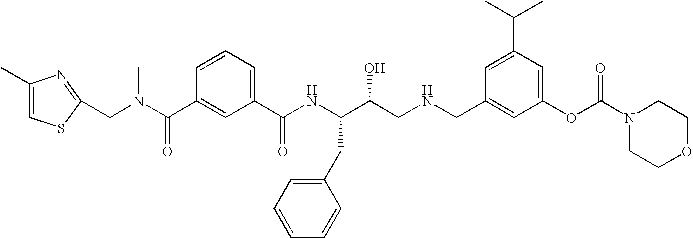

A mixture of 153 mg (0.326 mmol) of N1-((2S,3R)-4-azido-3-hydroxy-1-phenylbutan-2-yl)-N3-methyl-N3-((4-methylthiazol-2-yl)methyl)isophthalamide and 45.1 mg of 20% Pd(OH)₂ in 15 mL of MeOH was stirred at r.t. under H₂ balloon. After 2 h the mixture was filtered through Celite and concentrated. N1-(2S,3R)-4-amino-3-hydroxy-1-phenylbutan-2-yl)-N3-methyl-N3-((4-methylthiazol-2-yl)methyl)isophthalamide was used in the next reaction without further purification.

To a stirring solution of 63.7 mg (0.145 mmol) of N1-((2S,3R)-4-amino-3-hydroxy-1-phenylbutan-2-yl)-N3-methyl-N3-((4-methylthiazol-2-yl)methyl)isophthalamide in 5 mL of CH₃CN was added 37.3 mg (0.143 mmol) of 3-formyl-5-isopropylphenyl morpholine-4-carboxylate in 8 mL of CH₃CN, 16.2 mg (0.258 mmol) of NaBH₃CN, and glacial acetic acid to a pH=6. After the cloudy solution was stirred at r.t. for 39.5 h, sat. NaHCO₃ was added at 0° C. to pH=8-9. Water (10-15 mL) was added, and the aqueous layer was extracted with EtOAc. The organic layer was washed with 15 mL of water and 15 mL of brine, dried over Na₂SO₄, filtered, and concentrated. Purification by flash silica gel chromatography (5% MeOH/CHCl₃) provided the 21.3 mg of final inhibitor with some impurity and 1.3 mg of pure final inhibitor.

### Example 1.10.2

### N1-((2S,3R)-4-((5-acetylpyridin-3-yl)methylamino)-3-hydroxy-1-phenylbutan-2-yl)-N3-methyl-N3-((4-methylthiazol-2-yl)methyl)isophthalamide

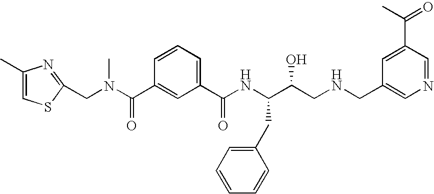

Following standard reductive amination procedure described, N1-((2S,3R)-4-amino-3-hydroxy-1-phenylbutan-2-yl)-N3-methyl-N3-((4-methylthiazol-2-yl)methyl)isophthalamide (112 mg, 0.63 mmol) and 5-acetylnicotinaldehyde (286 mg, 0.63 mmol) were reacted with NaB(OAc)₃ to provide N1-((2S,3R)-4-((5-acetylpyridin-3-yl)methylamino)-3-hydroxy-1-phenylbutan-2-yl)-N3-methyl-N3-((4-methylthiazol-2-yl)methyl)isophthalamide (167 mg) as an off white solid.

### Example 1.10.3

### 3-(((2R,3S)-2-hydroxy-3-(3-(methyl((4-methylthiazol-2-yl)methyl)carbamoyl)benzamido)-4-phenylbutylamino)methyl)-5-isopropylphenyl acetate

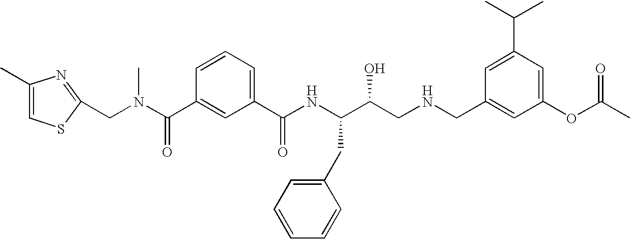

To a stirring solution of 58.4 mg (0.133 mmol) of N1-((2S,3R)-4-amino-3-hydroxy-1-phenylbutan-2-yl)-N3-methyl-N3-((4-methylthiazol-2-yl)methyl)isophthalamide and 27.3 mg (0.132 mmol) of 3-formyl-5-isopropylphenyl acetate in 5 mL of THF was added 10 μL of AcOH. After 3 h, 32.6 mg of NaBH(OAc)₃ was added, and after another 2 h, 36.5 mg of NaBH(OAc)₃ was added. The cloudy solution was stirred for 2.5 h, and EtOAc and 15 mL of H₂O were added. The organic layer was washed with 15 mL of water, and the aqueous layer was extracted with EtOAc (4×). The combined extracts were washed with brine, dried over Na₂SO₄, filtered, and concentrated. The crude product was purified by flash silica gel chromatography (5% MeOH/CHCl₃) (neutral silica gel) to provide 19.3 mg of final inhibitor with some impurity and 12.5 mg of pure 3-(((2R,3S)-2-hydroxy-3-(3-(methyl((4-methylthiazol-2-yl)methyl)carbamoyl)benzamido)-4-phenylbutylamino)methyl)-5-isopropylphenyl acetate.

### Example 1.10.4

### 3-(((2R,3S)-2-hydroxy-3-(3-(methyl((4-methylthiazol-2-yl)methyl)carbamoyl)benzamido)-4-phenylbutylamino)methyl)-5-isopropylphenyl dimethyl phosphate

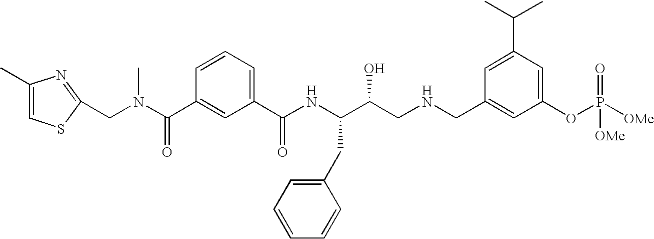

To a stirring solution of 39.1 mg (0.144 mmol) of 3-formyl-5-isopropylphenyl dimethyl phosphate in 5 mL of THF was added 75.7 mg (0.167 mmol) of the N1-((2S,3R)-4-amino-3-hydroxy-1-phenylbutan-2-yl)-N3-methyl-N3-((4-methylthiazol-2-yl)methyl)isophthalamide in 5 mL of THF followed by 15 μL (0.261 mmol) of AcOH. After stirring for 3 h, 37.6 mg of NaBH(OAc)₃ was added, and after another 2 h 33.3 mg of NaBH(OAc)₃ was added. After 2 h, H₂O and EtOAc were added, and the aqueous layer was extracted with EtOAc (4×). The combined extracts were washed with brine, dried over Na₂SO₄, filtered, and concentrated. Purification by flash silica gel chromatography (5% MeOH/CHCl₃) (neutral silica gel) provided 9.0 mg of pure 3-((2R,3S)-2-hydroxy-3-(3-(methyl((4-methylthiazol-2-yl)methyl)carbamoyl)benzamido)-4-phenylbutylamino)methyl)-5-isopropylphenyl dimethyl phosphate.

### Example 1.11

### Post-coupling Modifications

### Example 1.11.1

### N1-((2S,3R)-3-hydroxy-4-((3-methoxybenzyl)(methyl)amino)-1-phenylbutan-2-yl)-N3-methyl-N3-((4-methylthiazol-2-yl)methyl)isophthalamide

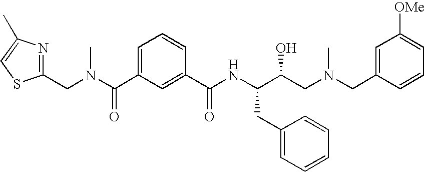

To a stirred solution of N1-(2S,3R)-3-hydroxy-4-(3-methoxybenzylamino)-1-phenylbutan-2-yl)-N3-methyl-N3-((4-methylthiazol-2-yl)methyl)isophthalamide (150 mg, 0.26 mmol) in CH₃CN (5 mL) was added 37% aqueous formaldehyde (0.10 mL, 1.3 mmol) and NaCNBH₃ (26 mg, 0.42 mmol). HOAc was added dropwise to the solution until it was neutral. The resulting mixture was stirred for 15 h and concentrated under reduced pressure. The residue was dissolved in EtOAc and saturated aqueous NaHCO₃. The layers were separated and the aqueous layer was extracted with EtOAc (2×10 mL). The combined organic layer was washed with brine, dried with Na₂SO₄ and concentrated under reduced pressure. The residue was purified by column chromatography (3% MeOH in CHCl₃) to provide title compound (100.6 mg, 66%) as a white solid.

### Example 1.11.2

### N1-((2S,3R)-3-hydroxy-4-(3-methoxybenzylamino)-1-phenylbutan-2-yl)-5-(hydroxyamino)-N3-methyl-N3-((4-methylthiazol-2-yl)methyl)isophthalamide

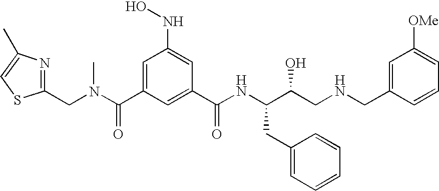

N₂H₄ (aq) (65% w/w) was added dropwise with vigorous stirring to a mixture of the starting material (0.018 g, 0.03 mmol, 1 eq) and 10% Pd/C (2.7 mg) in 1 mL THF. The reaction was stirred for at least 20 min and checked by TLC before adding additional N₂H₄. When the starting material and intermediate nitroso compound were almost completely reacted (TLC) the reaction was poured into water to quench. The mixture was extracted with EtOAc (×3). The combined organics were washed with water (×2), brine (×1), and dried over Na₂SO₄. The inorganics were filtered off, and the solvent was removed in vacuo. Purification via flash chromatography on silica gel yielded 6.1 mg (0.01 mmol, 34% yield) of the product.

### Example 1.11.3

### N1-((2S,3R)-3-hydroxy-4-(3-methoxybenzylamino)-1-phenylbutan-2-yl)-5-(hydroxyamino)-N3-((R)-1-phenylethyl)isophthalamide

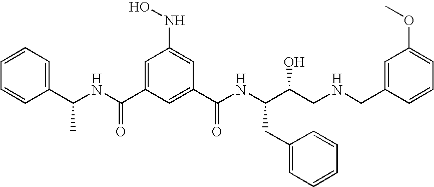

N1-((2S,3R)-3-hydroxy-4-(3-methoxybenzylamino)-1-phenylbutan-2-yl)-5-(hydroxyamino)-N3-((R)-1-phenylethyl)isophthalamide was synthesized from N1-((2S,3R)-3-hydroxy-4-(3-methoxybenzylamino)-1-phenylbutan-2-yl)-5-nitro-N3-((R)-1-phenylethyl)isophthalamide following the procedure described for N1-((2S,3R)-3-hydroxy-4-(3-methoxybenzylamino)-1-phenylbutan-2-yl)-5-(hydroxyamino)-N3-methyl-N3-((4-methylthiazol-2-yl)methyl)isophthalamide.

### Example 1.11.4

### 3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzoic acid

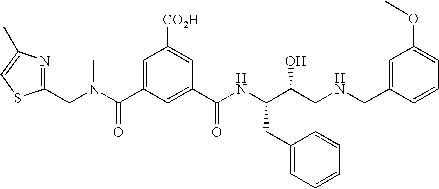

To a stirred solution of methyl 3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzoate (18 mg, 0.03 mmol) in THF (3 mL) was added 1N LiOH solution (1 mL). The resulting mixture was stirred for 3 h and the solvent was removed under reduced pressure. The residue was dissolved in H₂O (3 mL) and acidified to pH 6. The aqueous layer was extracted with EtOAc (3×3 mL). The combined organic layer was dried over Na₂SO₄ and concentrated under reduce pressure to provide title acid (13.7 mg, 76%) as a white solid.

### Example 1.11.5

### (S)-methyl 3-hydroxy-2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzamido)propanoate

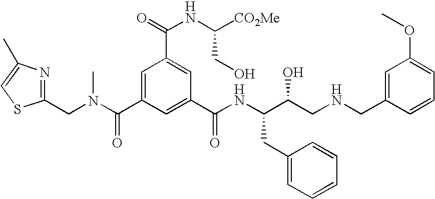

To a stirred solution of 3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzoic acid (109 mg, 0.18 mmol) and L-serine methyl ester hydrochloride (30 mg, 0.19 mmol) in CH₂Cl₂ (8 mL) was added HOBt (26 mg 0.19 mmol) and EDCI (36 mg, 0.19 mmol). The resulting mixture was stirred at room temperature for 15 h and quenched with water. The layers were separated and the aqueous layer was extracted with CH₂Cl₂ (2×10 mL). The combined organic layer was washed with brine, dried with Na₂SO₄ and concentrated under reduced pressure. The resulting oil was purified by column chromatography (20% MeOH/CHCl₃) to provide the product (47.7 mg, 37%) as a white solid.

### Example 1.11.6

### (S)-methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)-4,5-dihydrooxazole-4-carboxylate

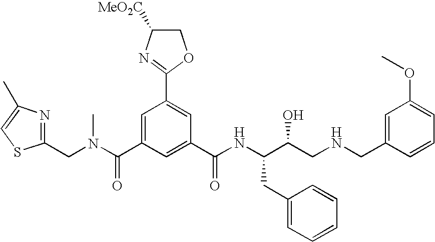

To a stirred solution of (S)-methyl 3-hydroxy-2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzamido)propanoate (34.4 mg, 0.05 mmol) in CH₂Cl₂ (3 mL) was added DAST (7 μL, 0.06 mmol) at −78° C. K₂CO₃ (11 mg, 0.08 mmol) was added after 1 h and the resulting mixture was warmed up to room temperature. The reaction was quenched with saturated aqueous NaHCO₃. The layers were separated and the aqueous layer was extracted with CH₂Cl₂ (2×5 mL). The combined organic layer was washed with brine, dried with Na₂SO₄ and concentrated under reduced pressure. The residue was purified by column chromatography (1.5% MeOH in CHCl₃) to provide title compound (22.0 mg, 65%) as an off-white solid.

### Example 1.11.7

### methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)oxazole-4-carboxylate

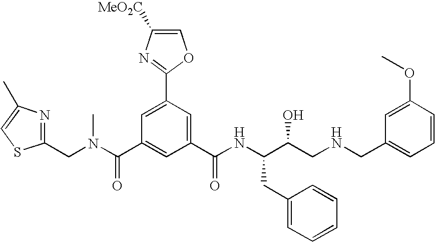

To a stirred solution of (S)-methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)-4,5-dihydrooxazole-4-carboxylate (20.3 mg, 0.03 mmol) in CH₂Cl₂ (1 mL) was added BrCCl₃ (12 μL, 0.12 mmol) and DBU (18 μL, 0.12 mmol) at 0° C. The resulting mixture was warmed up to room temperature and stirred for 15 h and was quenched with saturated aqueous NaHCO₃. The layers were separated and the aqueous layer was extracted with CH₂Cl₂ (2×5 mL). The combined organic layer was washed with brine, dried with Na₂SO₄ and concentrated under reduced pressure. The residue was purified by column chromatography (1% MeOH in CHCl₃) to provide title compound (11.1 mg, 55%) as an off white solid.

### Example 1.11.8

### N1-((2S,3R)-3-hydroxy-4-(3-hydroxybenzylamino)-1-phenylbutan-2-yl)-5-(N-methylmethylsulfonamido)-N3-((R)-1-phenylethyl)isophthalamide

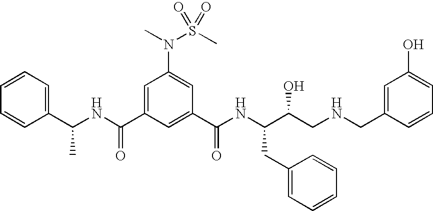

To a stirred solution of N1-((2S,3R)-3-hydroxy-4-(3-methoxybenzylamino)-1-phenylbutan-2-yl)-5-(N-methylmethylsulfonamido)-N3-((R)-1-phenylethyl)isophthalamide (51 mg, 0.08 mmol) in CH₂Cl₂ (5 mL) was added BBr₃ (1.0 M, 0.66 mL) at 0° C. The resulting mixture was warmed up to room temperature and stirred for 2 h and was quenched with saturated aqueous NaHCO₃. The layers were separated and the aqueous layer was extracted with CH₂Cl₂ (2×5 mL). The combined organic layer was washed with brine, dried with Na₂SO₄ and concentrated under reduced pressure. The residue was purified by column chromatography (15% MeOH in CHCl₃) to provide title compound (15.1 mg, 30%) as an off white solid.

### Example 1.11.9

### N1-((2S,3R)-4-(5-tert-butyl-2-hydroxybenzylamino)-3-hydroxy-1-phenylbutan-2-yl)-5-(N-methylmethylsulfonamido)-N3-((R)-1-phenylethyl)isophthalamide

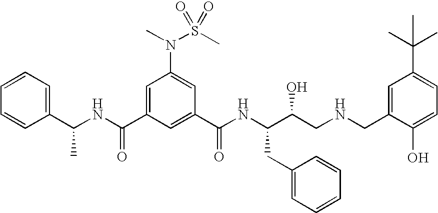

N1-((2S,3R)-4-(5-tert-butyl-2-hydroxybenzylamino)-3-hydroxy-1-phenylbutan-2-yl)-5-(N-methylmethylsulfonamido)-N3-((R)-1-phenylethyl)isophthalamide was obtained from TBS deprotection of N1-((2S,3R)-4-(5-tert-butyl-2-(tert-butyldimethylsilyloxy)benzylamino)-3-hydroxy-1-phenylbutan-2-yl)-5-(N-methylmethylsulfonamido)-N3-((R)-1-phenylethyl)isophthalamide.

### Example 1.11.10

### N1-((2S,3R)-4-(3-amino-5-isopropylbenzylamino)-3-hydroxy-1-phenylbutan-2-yl)-N3-methyl-N3-((4-methylthiazol-2-yl)methyl)isophthalamide

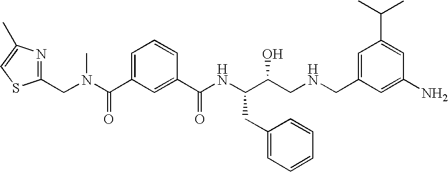

A mixture of 35.3 mg (0.0489 mmol) of the Cbz protected amine benzyl 3-(((2R,3S)-2-hydroxy-3-(3-(methyl((4-methylthiazol-2-yl)methyl)carbamoyl)benzamido)-4-phenylbutylamino)methyl)-5-isopropylphenylcarbamate and 12.2 mg of 20% Pd(OH)₂ in 10 mL of MeOH was stirred at r.t. under H₂ balloon. Additional 20% Pd(OH)₂ was added over a period of 6 h (94.1 mg in 3 portions). The mixture was filtered through Celite and concentrated. Purification by flash silica gel chromatography (15% MeOH/mL CHCl₃) provided 6.1 mg of the target compound as a pale yellow solid in 21.3% yield.

### Example 1.11.11

### N1-((2S,3R)-3-hydroxy-4-((5-(hydroxymethyl)-6-methylpyridin-2-yl)methylamino)-1-phenylbutan-2-yl)-N3-methyl-N3-((4-methylthiazol-2-yl)methyl)isophthalamide

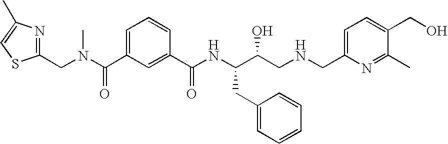

To a stirring solution of 52.6 mg (0.0893 mmol) of methyl 6-((2R,3S)-2-hydroxy-3-(3-(methyl((4-methylthiazol-2-yl)methyl)carbamoyl)benzamido)-4-phenylbutylamino)methyl)-2-methylnicotinate in 5 mL of THF at 0° C. was added 10.6 mg (0.279 mmol) of LiAlH₄. After 45 min, 10 μL of H₂O, 10 μL of 15% aqueous NaOH, and 30 μL of brine. EtOAc was added and stirring was continued at r.t. for 90 min. The mixture was filtered through Celite, and concentrated to give 9.9 mg of the target compound in 19.8% yield.

### Example 1.11.12

### 3-(((2R,3S)-2-hydroxy-3-(3-(methyl((4-methylthiazol-2-yl)methyl)carbamoyl)benzamido)-4-phenylbutylamino)methyl)-5-methoxybenzoic acid

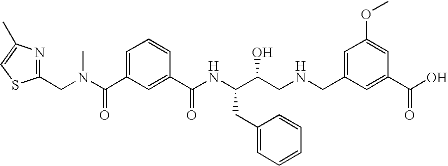

Methyl 3-(((2R,3S)-2-hydroxy-3-(3-(methyl((4-methylthiazol-2-yl)methyl)carbamoyl)benzamido)-4-phenylbutylamino)methyl)-5-methoxybenzoate (62 mg, 0.0983 mmol) in MeOH/water (15/2 mL) at rt was added NaOH (24 mg, 0.58 ml). The resulting mixture was stirred for several days and MeOH was removed in vacuo. The aqueous layer was acidified to pH=3-4 and water was removed in vacuo. Solid residue was dissolved into 10% MeOH/CHCl₃ and filtered, washed with the same solvent to get acid solution, which was concentrated in vacuo, purified with flash chromatography to give the desired product as a white solid (50 mg, 82% yield). ¹H NMR (300 MHz, CDCl₃+CD₃OD), d: 7.924-7.353 (m, 6H), 7.233-6.903 (m, 7H), 4.929 (br, 1.4H), 4.627 (br, 0.6H), 4.334 (m, 1H), 4.197 (m, 1H), 4.022 (m, 2H), 3.722 (s, 3H), 3.343-2.832 (m, 7H), 2.433 (s, 3H).

### Example 1.11.13

### N1-((2S,3R)-3-hydroxy-4-(3-methoxy-5-(methylcarbamoyl)benzylamino)-1-phenylbutan-2-yl)-N3-methyl-N3-((4-methylthiazol-2-yl)methyl)isophthalamide

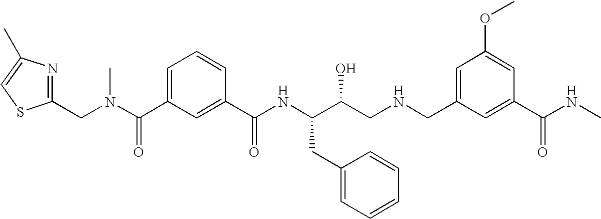

3-(((2R,3S)-2-hydroxy-3-(3-(methyl((4-methylthiazol-2-yl)methyl)carbamoyl)benzamido)-4-phenylbutylamino)methyl)-5-methoxybenzoic acid (37 mg, 0.06 mmol) and MeNH₂HCl (8.1 mg, 0.12 mmol) in DCM (20 mL) at rt was added triethylamine (1 mL, excess), followed by EDC (13.8 mg, 0.072 mmol) and HOBt (9.72 mg, 0.072 mmol). The resulting solution was stirred for 12 h and quenched with aqueous saturated NaHCO₃. The organic layer was separated and extracted with chloroform three times, dried (MgSO₄) and concentrated in vacuo, purified with flash chromatography to give the desired product as a white solid (25 mg). ¹H NMR (300 MHz, CDCl₃+CD₃OD), d: 7.675-7.389 (m, 3H), 7.330-7.167 (m, 9H), 6.970 (s, 1H), 4.964 (br, 1.4H), 4.646 (br, 0.6H), 4.310 (m, 1H), 3.909-3.752 (m, 6H), 3.111-2.792 (m, 10H), 2.452 (s, 3H).

### Example 1.11.14

### 3-(((2R,3S)-2-hydroxy-3-(3-(N-methylmethylsulfonamido)-5-((R)-1-phenylethylcarbamoyl)benzamido)-4-phenylbutylamino)methyl)benzoic acid

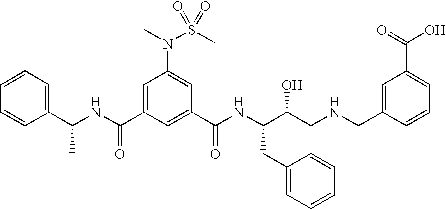

Methyl 3-(((2R,3S)-2-hydroxy-3-(3-(N-methylmethylsulfonamido)-5-((R)-1-phenylethylcarbamoyl)benzamido)-4-phenylbutylamino)methyl)benzoate (126 mg, 0.183 mmol) in MeOH/THF/water (6/6/1 mL) at rt was added NaOH (51.4 mg, 1.28 mmol). The resulting mixture was heated to 50° C. and stirred for 2 h and the organic solvent was removed in vacuo. The aqueous layer was acidified to pH=3-4 and water was removed in vacuo. Solid residue was dissolved into 10% MeOH/CHCl₃ and filtered, washed with the same solvent to get acid solution, which was concentrated in vacuo, purified with flash chromatography to give the desired product as a white solid (80 mg). ¹H NMR (300 MHz, CDCl₃+CD₃OD), d: 8.242 (s, 2H), 8.102 (s, 1H), 7.841 (s, 2H), 7.727 (s, 1H), 7.383-7.084 (m, 11H), 5.197 (br, 1H), 4.148 (br, 3H), 3.631-3.485 (m, 2H), 3.335-3.011 (m, 2H), 3.176 (s, 3H), 2.876 (m, 1 H), 2.761 (s, 3H), 1.537 (m, 3H).

### Example 1.11.15

### N1-((2S,3R)-3-hydroxy-4-(3-(hydroxymethyl)benzylamino)-1-phenylbutan-2-yl)-5-(N-methylmethylsulfonamido)-N3-((R)-1-phenylethyl)isophthalamide

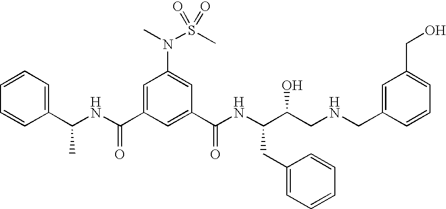

Methyl 3-(((2R,3S)-2-hydroxy-3-(3-(N-methylmethylsulfonamido)-5-((R)-1-phenylethylcarbamoyl)benzamido)-4-phenylbutylamino)methyl)benzoate (115 mg, 0.167 mmol) in DCM (20 mL) at −78° C. was added Dibal-H (1.5 M in toluene, 0.78 mL, 1.172 mmol), stirred at the same temperature for 2 h and warmed to −30° C. for 1 h. Then the reaction was quenched with saturated aqueous NH₄Cl and stirred for a couple of hours. Anhydrous Na₂SO₄ was added to make the mixture clear two phases, filtered and washed with EtOAc. The combined organic solution was concentrated to get the crude product which was purified with flash chromatography to give the desired product as a white solid. ¹H NMR (300 MHz, CDCl₃+CD₃OD), d: 8.110 (s, 1H), 7.912 (s, 1H), 7.761 (s, 1H), 7.431-7.127 (m, 14H), 5.283 (m, 1H), 4.567 (m 2H), 4.264 (m, 1H), 3.880 (m, 3H), 3.284 (s, 3H), 3.092 (m, 1H), 2.908-2.796 (m, 6H), 1.619 (d, J=6.9 Hz, 3H).

### Example 1.11.16

### N1-((2S,3R)-3-hydroxy-1-phenyl-4-((6-(trifluoromethyl)pyridin-3-yl)methylamino)butan-2-yl)-N3-methyl-N3-((4-methylthiazol-2-yl)methyl)isophthalamide

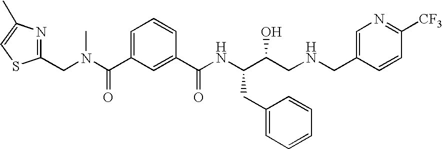

Tert-butyl (2S,3R)-3-hydroxy-1-phenyl-4-((6-(trifluoromethyl)pyridin-3-yl)methylamino)butan-2-ylcarbamate (60 mg, 0.137 mmol) in DCM (5 mL) was added TFA (1 mL) at room temperature and stirred for 45 min. The solvent was removed in vacuo and dissolved in DCM, then diisopropylethylamine (0.5 mL, excess), 3-(methyl((4-methylthiazol-2-yl)methyl)carbamoyl)benzoic acid (41.6 mg, 0.143 mmol), EDC (28.7 mg, 0.15 mmol) and HOBt (20.3 mg, 0.15 mmol) were added successively to the reaction. The resulting solution was stirred at room temperature fro overnight. The reaction was quenched with diluted aqueous NaHCO₃, separated, extracted with chloroform. The combined organic solvent was removed in vacuo and purified by silica gel chromatography to afford final compound. ¹H NMR (300 MHz, CDCl₃+CD₃OD), d: 8.649 (s, 1H), 7.880-7.164 (m, 11H), 6.891 (s, 1H), 4.947 (br, 1.3H), 4.639 (br, 0.7H), 4.336 (m, 1H), 3.924-3.827 (m, 2H), 3.723 (m, 1H), 2.989 (s 3H), 3.092-2.890 (m, 2H), 2.753 (s, 2H), 2.437 (s, 3H).

### Example 1.11.17

### 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoic acid

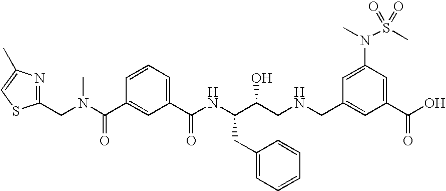

A solution of 44.5 mg of methyl 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoate and 0.24 mL of 2N NaOH in 2 mL of MeOH and 2 mL of THF was stirred at 50° C. for 2 h. The solution was concentrated, water was added, and the pH was adjusted to pH=4 with 1N HCl. The aqueous layer was extracted with the extract of (40 mL CHCl₃: 5 mL of H₂O: 5 mL of MeOH) (2×). The combined extracts were dried over Na₂SO₄, filtered, and concentrated. Purification by flash silica gel chromatography (50% MeOH/CHCl₃) was not successful. Some of the product was dissolved in CHCl₃, and precipitated with the addition of hexanes. The colorless solid was collected by filtration to give 21.1 mg of 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoic acid as a colorless solid.

### Example 2

### Inhibition of Memapsin 2 Beta-Secretase Activity

Potency of compounds were determined by measurement of their inhibition of memapsin 2 activity toward a fluorescent substrate. Kinetic inhibition experiments were performed using the procedure as described in Ermolieff, et al. (Biochemistry 39:12450-12456 (2000), the teachings of which are incorporated hereby in their entirety). Briefly, assays were performed at pH 4, 37° C., by pre-incubation of memapsin 2 enzyme with compound for 20 minutes. Activity measurements were initiated by addition of a fluorogenic substrate FS-2 (Bachem Americas, Torrance, Calif.) MCA-SEVNLDAEFR-DNP (SEQ ID NO.: 2). The substrate was derived from 10 amino acids of the human amyloid precursor protein (APP), with the Swedish variant amino acids at the beta-secretase cleavage site. The terminal amino acid was modified from arginine to lysine to facilitate derivatization with a functional group for detection by autofluorescence. The amino acid sequence of the “core” peptide of the substrate is SEVNLDAEFK (SEQ ID NO.: 3). The amino terminus was derivatized with (7-methoxycoumarin-4-yl)acetyl (MCA), and the epsilon amine of the lysine side chain of the terminal residue (K in sequence SEVNLDAEFK (SEQ ID NO.: 3)) was derivatized with 2,4-dinitorphenyl (DNP).

Fluorescent signal increase over time was measured as a rate of hydrolysis of the peptide substrate. Inhibition of hydrolytic rate was expressed relative to uninhibited controls and fit to a model for tight-binding inhibitors (J. Bieth, in “Proteinase Inhibitors,” Bayer Symposium V, 463-469, 1974, which is herein incorporated by reference for all purposes in its entirety). The results are presented in Table 1 below.

TABLE 1
Compound inhibition data.

| | | M2Ki | CDKi | M1Ki | IC50 |
|---|---|---|---|---|---|
| Ref # | Structure | nM | nM | nM | μM |
| 1 | N1-((2S,3R)-3-hydroxy-4-(3- | +++ | | | +++ |
| | methoxybenzylamino)-1-phenylbutan-2-yl)-N3,5- | | | | |
| | dimethyl-N3-((4-methylthiazol-2- | | | | |
| | yl)methyl)isophthalamide | | | | |
| 2 | 5-tert-butyl-N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2- | | | | |
| | yl)methyl)isophthalamide | | | | |
| 3 | N1-((2S,3R)-3-hydroxy-4-(3- | +++ | +++ | ++ | +++ |
| | methoxybenzylamino)-1-phenylbutan-2-yl)-5- | | | | |
| | methoxy-N3-methyl-N3-((4-methylthiazol-2- | | | | |
| | yl)methyl)isophthalamide | | | | |
| 4 | 5-fluoro-N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2- | | | | |
| | yl)methyl)isophthalamide | | | | |
| 5 | N1-((2S,3R)-3-hydroxy-4-((4-isopropylpyridin-2- | +++ | | | +++ |
| | yl)methylamino)-1-phenylbutan-2-yl)-N3-methyl- | | | | |
| | N3-((4-methylthiazol-2-yl)methyl)isophthalamide | | | | |
| 6 | N1-((2S,3R)-3-hydroxy-4-(3- | + | | | + |
| | methoxybenzylamino)-1-phenylbutan-2-yl)-N3- | | | | |
| | methyl-N3-((5-methylthiazol-2- | | | | |
| | yl)methyl)isophthalamide | | | | |
| 7 | N1-((2S,3R)-3-hydroxy-1-phenyl-4-((6- | + | | | |
| | (trifluoromethyl)pyridin-3-yl)methylamino)butan- | | | | |
| | 2-yl)-N3-methyl-N3-((4-methylthiazol-2- | | | | |
| | yl)methyl)isophthalamide | | | | |
| 8 | N1-((2S,3R)-4-(3,5-dimethoxybenzylamino)-3- | +++ | +++ | ++ | +++ |
| | hydroxy-1-phenylbutan-2-yl)-N3-methyl-N3-((4- | | | | |
| | methylthiazol-2-yl)methyl)isophthalamide | | | | |
| 9 | N1-cyclopropyl-N3-((2S,3R)-3-hydroxy-4-(3- | +++ | | | +++ |
| | methoxybenzylamino)-1-phenylbutan-2-yl)-5- | | | | |
| | methyl-N1-((4-methylthiazol-2- | | | | |
| | yl)methyl)isophthalamide | | | | |
| 10 | N1-((2S,3R)-3-hydroxy-4-((6-isopropylpyridin-2- | +++ | | | +++ |
| | yl)methylamino)-1-phenylbutan-2-yl)-N3-methyl- | | | | |
| | N3-((4-methylthiazol-2-yl)methyl)isophthalamide | | | | |
| 11 | N1-((2S,3R)-3-hydroxy-4-(3- | +++ | | | +++ |
| | methoxybenzylamino)-1-phenylbutan-2-yl)-N3- | | | | |
| | ((4-methylthiazol-2-yl)methyl)-N3- | | | | |
| | propylisophthalamide | | | | |
| 12 | N1-((2S,3R)-4-(3-cyclopropylbenzylamino)-3- | +++ | | | +++ |
| | hydroxy-1-phenylbutan-2-yl)-N3-methyl-N3-((4- | | | | |
| | methylthiazol-2-yl)methyl)isophthalamide | | | | |
| 13 | N1-((2S,3R)-4-(3,4-dimethylbenzylamino)-3- | +++ | | | |
| | hydroxy-1-phenylbutan-2-yl)-N3-methyl-N3-((4- | | | | |
| | methylthiazol-2-yl)methyl)isophthalamide | | | | |
| 14 | N1-(furan-2-ylmethyl)-N3-((2S,3R)-3-hydroxy-4- | + | | | |
| | (3-methoxybenzylamino)-1-phenylbutan-2-yl)-N1- | | | | |
| | methylisophthalamide | | | | |
| 15 | N1-((2S,3R)-3-hydroxy-1-phenyl-4-(pyridin-3- | + | | | |
| | ylmethylamino)butan-2-yl)-N3-methyl-N3-((4- | | | | |
| | methylthiazol-2-yl)methyl)isophthalamide | | | | |
| 16 | N1-ethyl-N3-((2S,3R)-3-hydroxy-4-(3- | +++ | | | +++ |
| | methoxybenzylamino)-1-phenylbutan-2-yl)-5- | | | | |
| | methoxy-N1-((4-methylthiazol-2- | | | | |
| | yl)methyl)isophthalamide | | | | |
| 17 | methyl 3-((2S,3R)-3-hydroxy-4-(3- | +++ | +++ | + | +++ |
| | methoxybenzylamino)-1-phenylbutan-2- | | | | |
| | ylcarbamoyl)-5-(methyl((4-methylthiazol-2- | | | | |
| | yl)methyl)carbamoyl)benzoate | | | | |
| 18 | N1-((2S,3R)-3-hydroxy-4-(3- | ++ | | | +++ |
| | methoxybenzylamino)-1-phenylbutan-2-yl)-N3- | | | | |
| | methyl-N3-((5-methylfuran-2- | | | | |
| | yl)methyl)isophthalamide | | | | |
| 19 | 3-((2S,3R)-3-hydroxy-4-(3-methoxybenzylamino)- | +++ | | | +++ |
| | 1-phenylbutan-2-ylcarbamoyl)-5-(methyl((4- | | | | |
| | methylthiazol-2-yl)methyl)carbamoyl)benzoic acid | | | | |
| 20 | N1-((2S,3R)-3-hydroxy-4-(3- | +++ | | | |
| | methoxybenzylamino)-1-phenylbutan-2-yl)-5- | | | | |
| | methoxy-N3-((4-methylthiazol-2-yl)methyl)-N3- | | | | |
| | propylisophthalamide | | | | |
| 21 | N1-((2S,3R)-3-hydroxy-4-(3- | +++ | +++ | + | +++ |
| | methoxybenzylamino)-1-phenylbutan-2-yl)-N3- | | | | |
| | methyl-N3-((4-methylthiazol-2-yl)methyl)-5- | | | | |
| | vinylisophthalamide | | | | |
| 22 | N1-((2S,3R)-3-hydroxy-1-phenyl-4-((5-(prop-1- | +++ | | | +++ |
| | en-2-yl)pyridin-3-yl)methylamino)butan-2-yl)-N3- | | | | |
| | methyl-N3-((4-methylthiazol-2- | | | | |
| | yl)methyl)isophthalamide | | | | |
| 23 | N1-((2S,3R)-3-hydroxy-1-phenyl-4-((5- | +++ | | | +++ |
| | (trifluoromethyl)pyridin-3-yl)methylamino)butan- | | | | |
| | 2-yl)-N3-methyl-N3-((4-methylthiazol-2- | | | | |
| | yl)methyl)isophthalamide | | | | |
| 24 | N1-((4,5-dimethylthiazol-2-yl)methyl)-N3- | + | | | |
| | ((2S,3R)-3-hydroxy-4-(3-methoxybenzylamino)-1- | | | | |
| | phenylbutan-2-yl)-N1-methylisophthalamide | | | | |
| 25 | N1-((2S,3R)-3-hydroxy-4-(3- | +++ | | | +++ |
| | methoxybenzylamino)-1-phenylbutan-2-yl)-N3- | | | | |
| | isopropyl-5-methoxy-N3-((4-methylthiazol-2- | | | | |
| | yl)methyl)isophthalamide | | | | |
| 26 | N1-((2S,3R)-3-hydroxy-1-phenyl-4-(pyridin-4- | + | | | + |
| | ylmethylamino)butan-2-yl)-N3-methyl-N3-((4- | | | | |
| | methylthiazol-2-yl)methyl)isophthalamide | | | | |
| 27 | N1-((2S,3R)-4-(3,5- | +++ | | | +++ |
| | bis(trifluoromethyl)benzylamino)-3-hydroxy-1- | | | | |
| | phenylbutan-2-yl)-N3-methyl-N3-((4- | | | | |
| | methylthiazol-2-yl)methyl)isophthalamide | | | | |
| 28 | N1-((2S,3R)-4-(3-fluoro-5- | +++ | | | +++ |
| | (trifluoromethyl)benzylamino)-3-hydroxy-1- | | | | |
| | phenylbutan-2-yl)-N3-methyl-N3-((4- | | | | |
| | methylthiazol-2-yl)methyl)isophthalamide | | | | |
| 29 | N1-cyclopropyl-N3-((2S,3R)-3-hydroxy-1-phenyl- | +++ | +++ | ++ | +++ |
| | 4-(3-(trifluoromethyl)benzylamino)butan-2-yl)- | | | | |
| | N1-((4-methylthiazol-2-yl)methyl)isophthalamide | | | | |
| 30 | N1-((2S,3R)-4-((5-cyclopropylpyridin-3- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 31 | N1-((2S,3R)-3-hydroxy-1-phenyl-4-(3- | +++ | | | +++ |
| | (trifluoromethyl)benzylamino)butan-2-yl)-5- | | | | |
| | methoxy-N3-methyl-N3-((4-methylthiazol-2- | | | | |
| | yl)methyl)isophthalamide | | | | |
| 32 | N1-((2S,3R)-3-hydroxy-4-((5-isopropylpyridin-3- | +++ | +++ | +++ | +++ |
| | yl)methylamino)-1-phenylbutan-2-yl)-N3,5- | | | | |
| | dimethyl-N3-((4-methylthiazol-2- | | | | |
| | yl)methyl)isophthalamide | | | | |
| 33 | N1-((2S,3R)-3-hydroxy-4-((2-isopropylpyridin-4- | +++ | +++ | ++ | +++ |
| | yl)methylamino)-1-phenylbutan-2-yl)-N3-methyl- | | | | |
| | N3-((4-methylthiazol-2-yl)methyl)isophthalamide | | | | |
| 34 | 5-amino-N1-((2S,3R)-3-hydroxy-4-(3- | + | | | ++ |
| | methoxybenzylamino)-1-phenylbutan-2-yl)-N3- | | | | |
| | methyl-N3-((4-methylthiazol-2- | | | | |
| | yl)methyl)isophthalamide | | | | |
| 35 | N1-((2S,3R)-3-hydroxy-4-(naphthalen-1- | ++ | | | |
| | ylmethylamino)-1-phenylbutan-2-yl)-N3-methyl- | | | | |
| | N3-((4-methylthiazol-2-yl)methyl)isophthalamide | | | | |
| 36 | methyl 6-(((2R,3S)-2-hydroxy-3-(3-(methyl((4- | + | | | |
| | methylthiazol-2-yl)methyl)carbamoyl)benzamido)- | | | | |
| | 4-phenylbutylamino)methyl)-2-methylnicotinate | | | | |
| 37 | N1-((2S,3R)-3-hydroxy-4-((5-(hydroxymethyl)-6- | + | | | |
| | methylpyridin-2-yl)methylamino)-1-phenylbutan- | | | | |
| | 2-yl)-N3-methyl-N3-((4-methylthiazol-2- | | | | |
| | yl)methyl)isophthalamide | | | | |
| 38 | 5-hydroxy-N1-((2S,3R)-3-hydroxy-4-(3- | +++ | | | |
| | methoxybenzylamino)-1-phenylbutan-2-yl)-N3- | | | | |
| | methyl-N3-((4-methylthiazol-2- | | | | |
| | yl)methyl)isophthalamide | | | | |
| 39 | N1-((2S,3R)-4-((1H-indol-7-yl)methylamino)-3- | +++ | | | |
| | hydroxy-1-phenylbutan-2-yl)-N3-methyl-N3-((4- | | | | |
| | methylthiazol-2-yl)methyl)isophthalamide | | | | |
| 40 | N1-((2S,3R)-3-hydroxy-4-((5-isopropylpyridin-3- | +++ | +++ | +++ | +++ |
| | yl)methylamino)-1-phenylbutan-2-yl)-5-methoxy- | | | | |
| | N3-methyl-N3-((4-methylthiazol-2- | | | | |
| | yl)methyl)isophthalamide | | | | |
| 41 | N1-cyclopropyl-N3-((2S,3R)-3-hydroxy-4-((5- | +++ | +++ | +++ | +++ |
| | isopropylpyridin-3-yl)methylamino)-1- | | | | |
| | phenylbutan-2-yl)-5-methyl-N1-((4-methylthiazol- | | | | |
| | 2-yl)methyl)isophthalamide | | | | |
| 42 | N3-((2S,3R)-3-hydroxy-4-(3- | + | | | |
| | methoxybenzylamino)-1-phenylbutan-2-yl)-N1,4- | | | | |
| | dimethyl-N1-((4-methylthiazol-2- | | | | |
| | yl)methyl)isophthalamide and N1-((2S,3R)-3- | | | | |
| | hydroxy-4-(3-methoxybenzylamino)-1- | | | | |
| | phenylbutan-2-yl)-N3,4-dimethyl-N3-((4- | | | | |
| | methylthiazol-2-yl)methyl)isophthalamide | | | | |
| | (regioisomeric mixture) | | | | |
| 43 | N1-((2S,3R)-3-hydroxy-1-phenyl-4-(1-(pyridin-3- | + | | | |
| | yl)ethylamino)butan-2-yl)-N3-methyl-N3-((4- | | | | |
| | methylthiazol-2-yl)methyl)isophthalamide | | | | |
| 44 | N1-((2S,3R)-3-hydroxy-4-(3- | +++ | | | +++ |
| | methoxybenzylamino)-1-phenylbutan-2-yl)-N3-(2- | | | | |
| | hydroxyethyl)-N5-methyl-N5-((4-methylthiazol-2- | | | | |
| | yl)methyl)benzene-1,3,5-tricarboxamide | | | | |
| 45 | N1-((2S,3R)-3-hydroxy-4-(3- | + | | | |
| | methoxybenzylamino)-1-phenylbutan-2-yl)-N3- | | | | |
| | methyl-N3-(pyridin-2-ylmethyl)isophthalamide | | | | |
| 46 | 5-(4,5-dihydrooxazol-2-yl)-N1-((2S,3R)-3- | +++ | ++ | + | +++ |
| | hydroxy-4-(3-methoxybenzylamino)-1- | | | | |
| | phenylbutan-2-yl)-N3-methyl-N3-((4- | | | | |
| | methylthiazol-2-yl)methyl)isophthalamide | | | | |
| 47 | N1-((2S,3R)-4-((5-(dimethylamino)pyridin-3- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 48 | N1-((2S,3R)-3-hydroxy-4-((4-methoxypyrimidin- | + | | | |
| | 2-yl)methylamino)-1-phenylbutan-2-yl)-N3- | | | | |
| | methyl-N3-((4-methylthiazol-2- | | | | |
| | yl)methyl)isophthalamide | | | | |
| 49 | N1-((2S,3R)-3-hydroxy-4-(3- | +++ | | | +++ |
| | methoxybenzylamino)-1-phenylbutan-2-yl)-5- | | | | |
| | (hydroxymethyl)-N3-methyl-N3-((4- | | | | |
| | methylthiazol-2-yl)methyl)isophthalamide | | | | |
| 50 | N1-((2S,3R)-4-(2-fluoro-6-methoxybenzylamino)- | +++ | | | +++ |
| | 3-hydroxy-1-phenylbutan-2-yl)-N3-methyl-N3- | | | | |
| | ((4-methylthiazol-2-yl)methyl)isophthalamide | | | | |
| 51 | N1-cyclopropyl-N3-((2S,3R)-3-hydroxy-1-phenyl- | +++ | | | +++ |
| | 4-(3-(trifluoromethoxy)benzylamino)butan-2-yl)- | | | | |
| | N1-((4-methylthiazol-2-yl)methyl)isophthalamide | | | | |
| 52 | N1-((2S,3R)-4-(3-tert-butylbenzylamino)-3- | +++ | | | +++ |
| | hydroxy-1-phenylbutan-2-yl)-N3-methyl-N3-((4- | | | | |
| | methylthiazol-2-yl)methyl)isophthalamide | | | | |
| 53 | N1-((2S,3R)-3-hydroxy-4-((2-isopropylpyridin-4- | +++ | | | +++ |
| | yl)methylamino)-1-phenylbutan-2-yl)-5-methoxy- | | | | |
| | N3-methyl-N3-((4-methylthiazol-2- | | | | |
| | yl)methyl)isophthalamide | | | | |
| 54 | methyl 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5-methoxybenzoate | | | | |
| 55 | N1-((2S,3R)-3-hydroxy-4-(3- | + | | | |
| | methoxybenzylamino)-1-phenylbutan-2-yl)-N3- | | | | |
| | methyl-N3-(pyridin-3-ylmethyl)isophthalamide | | | | |
| 56 | N1-((2S,3R)-4-((2-fluoro-5-isopropylpyridin-3- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 57 | N1-((2S,3R)-3-hydroxy-4-(3- | +++ | | | |
| | methoxybenzylamino)-1-phenylbutan-2-yl)-N3-(2- | | | | |
| | hydroxyethyl)-5-methoxy-N3-((4-methylthiazol-2- | | | | |
| | yl)methyl)isophthalamide | | | | |
| 58 | N1-((2S,3R)-4-(3-tert-butylbenzylamino)-3- | +++ | | | +++ |
| | hydroxy-1-phenylbutan-2-yl)-5-(N- | | | | |
| | methylmethylsulfonamido)-N3-((R)-1- | | | | |
| | phenylethyl)isophthalamide | | | | |
| 59 | N1-cyclopropyl-N3-((2S,3R)-3-hydroxy-1-phenyl- | +++ | | | +++ |
| | 4-((5-(trifluoromethyl)pyridin-3- | | | | |
| | yl)methylamino)butan-2-yl)-N1-((4-methylthiazol- | | | | |
| | 2-yl)methyl)isophthalamide | | | | |
| 60 | N1-((2S,3R)-3-hydroxy-4-((2-isopropylpyridin-4- | +++ | | | +++ |
| | yl)methylamino)-1-phenylbutan-2-yl)-N3,5- | | | | |
| | dimethyl-N3-((4-methylthiazol-2- | | | | |
| | yl)methyl)isophthalamide | | | | |
| 61 | N1-((2S,3R)-3-hydroxy-4-(3- | +++ | | | ++ |
| | methoxybenzylamino)-1-phenylbutan-2-yl)-N3- | | | | |
| | methyl-N3-((1-methyl-1H-pyrazol-3- | | | | |
| | yl)methyl)isophthalamide | | | | |
| 62 | (S)-methyl 3-hydroxy-2-(3-((2S,3R)-3-hydroxy-4- | +++ | | | |
| | (3-methoxybenzylamino)-1-phenylbutan-2- | | | | |
| | ylcarbamoyl)-5-(methyl((4-methylthiazol-2- | | | | |
| | yl)methyl)carbamoyl)benzamido)propanoate | | | | |
| 63 | (S)-methyl 2-(3-((2S,3R)-3-hydroxy-4-(3- | + | | | |
| | methoxybenzylamino)-1-phenylbutan-2- | | | | |
| | ylcarbamoyl)-5-(methyl((4-methylthiazol-2- | | | | |
| | yl)methyl)carbamoyl)phenyl)-4,5-dihydrooxazole- | | | | |
| | 4-carboxylate | | | | |
| 64 | N1-((2S,3R)-3-hydroxy-1-phenyl-4-(pyridin-2- | + | | | ++ |
| | ylmethylamino)butan-2-yl)-N3-methyl-N3-((4- | | | | |
| | methylthiazol-2-yl)methyl)isophthalamide | | | | |
| 65 | 5-(dimethylamino)-N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2- | | | | |
| | yl)methyl)isophthalamide | | | | |
| 66 | methyl 2-(3-((2S,3R)-3-hydroxy-4-(3- | + | | | |
| | methoxybenzylamino)-1-phenylbutan-2- | | | | |
| | ylcarbamoyl)-5-(methyl((4-methylthiazol-2- | | | | |
| | yl)methyl)carbamoyl)phenyl)oxazole-4- | | | | |
| | carboxylate | | | | |
| 67 | 3-((2S,3R)-3-hydroxy-4-(3-methoxybenzylamino)- | +++ | | | +++ |
| | 1-phenylbutan-2-ylcarbamoyl)-5-(methyl((4- | | | | |
| | methylthiazol-2-yl)methyl)carbamoyl)phenyl | | | | |
| | methanesulfonate | | | | |
| 68 | N1-((2S,3R)-3-hydroxy-4-((5-isopentylpyridin-3- | +++ | | | +++ |
| | yl)methylamino)-1-phenylbutan-2-yl)-N3-methyl- | | | | |
| | N3-((4-methylthiazol-2-yl)methyl)isophthalamide | | | | |
| 69 | N1-((2S,3R)-3-hydroxy-4-(3- | + | | | |
| | methoxybenzylamino)-1-phenylbutan-2-yl)-5- | | | | |
| | methoxy-N3-((R)-1-phenylethyl)isophthalamide | | | | |
| 70 | 3-((2S,3R)-3-hydroxy-4-(3-methoxybenzylamino)- | +++ | | | +++ |
| | 1-phenylbutan-2-ylcarbamoyl)-5-((R)-1- | | | | |
| | phenylethylcarbamoyl)phenyl methanesulfonate | | | | |
| 71 | N1-((2S,3R)-3-hydroxy-4-(3- | +++ | + | + | +++ |
| | hydroxybenzylamino)-1-phenylbutan-2-yl)-5-(N- | | | | |
| | methylmethylsulfonamido)-N3-((R)-1- | | | | |
| | phenylethyl)isophthalamide | | | | |
| 72 | 3-(((2R,3S)-2-hydroxy-3-(3-(methyl((4- | +++ | + | + | ++ |
| | methylthiazol-2-yl)methyl)carbamoyl)benzamido)- | | | | |
| | 4-phenylbutylamino)methyl)-5-methoxybenzoic | | | | |
| | acid | | | | |
| 73 | N1-((2S,3R)-3-hydroxy-4-(3- | + | | | + |
| | hydroxybenzylamino)-1-phenylbutan-2-yl)-N3- | | | | |
| | methyl-N3-((4-methylthiazol-2- | | | | |
| | yl)methyl)isophthalamide | | | | |
| 74 | benzyl 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5- | | | | |
| | isopropylphenylcarbamate | | | | |
| 75 | N1-((2S,3R)-4-(3-amino-5- | +++ | | | +++ |
| | isopropylbenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 76 | N1-((2S,3R)-3-hydroxy-4-(3- | +++ | | | |
| | methoxybenzylamino)-1-phenylbutan-2-yl)-N3- | | | | |
| | methyl-5-(methylamino)-N3-((4-methylthiazol-2- | | | | |
| | yl)methyl)isophthalamide | | | | |
| 77 | N1-((2S,3R)-4-(3-tert-butyl-2- | +++ | | | +++ |
| | hydroxybenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 78 | N3-((2S,3R)-3-hydroxy-4-(3- | +++ | | | +++ |
| | methoxybenzylamino)-1-phenylbutan-2-yl)-N5- | | | | |
| | methyl-N5-((4-methylthiazol-2- | | | | |
| | yl)methyl)biphenyl-3,5-dicarboxamide | | | | |
| 79 | N1-((2S,3R)-4-(3-tert-butyl-2- | +++ | | | +++ |
| | hydroxybenzylamino)-3-hydroxy-1-phenylbutan- | | | | |
| | 2-yl)-5-(N-methylmethylsulfonamido)-N3-((R)-1- | | | | |
| | phenylethyl)isophthalamide | | | | |
| 80 | N1-((2S,3R)-3-hydroxy-4-(3- | + | | | |
| | methoxybenzylamino)-1-phenylbutan-2-yl)-N3-(4- | | | | |
| | methylthiazol-2-yl)isophthalamide | | | | |
| 81 | N1-((2S,3R)-3-hydroxy-1-phenyl-4-((5- | +++ | + | +++ | +++ |
| | (trifluoromethyl)pyridin-3-yl)methylamino)butan- | | | | |
| | 2-yl)-5-(N-methylmethylsulfonamido)-N3-((R)-1- | | | | |
| | phenylethyl)isophthalamide | | | | |
| 82 | N1-((2S,3R)-3-hydroxy-4-((5-isopropylpyridin-3- | +++ | ++ | ++ | +++ |
| | yl)methylamino)-1-phenylbutan-2-yl)-N3-methyl- | | | | |
| | N3-((R)-1-(4-methylthiazol-2- | | | | |
| | yl)ethyl)isophthalamide | | | | |
| 83 | N1-((2S,3R)-3-hydroxy-4-((2-isopropylpyridin-4- | +++ | | | +++ |
| | yl)methylamino)-1-phenylbutan-2-yl)-5-methoxy- | | | | |
| | N3-((4-methylthiazol-2-yl)methyl)isophthalamide | | | | |
| 84 | N1-((2S,3R)-3-hydroxy-4-(3- | + | | | |
| | methoxybenzylamino)-1-phenylbutan-2-yl)-N3- | | | | |
| | methyl-N3-(4-methylthiazol-2-yl)isophthalamide | | | | |
| 85 | N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2-yl)methyl)-5- | | | | |
| | (oxazol-2-yl)isophthalamide | | | | |
| 86 | N1-((2S,3R)-4-(3-chlorobenzylamino)-3-hydroxy- | +++ | ++ | +++ | +++ |
| | 1-phenylbutan-2-yl)-5-(N- | | | | |
| | methylmethylsulfonamido)-N3-((R)-1- | | | | |
| | phenylethyl)isophthalamide | | | | |
| 87 | N1-((2S,3R)-3-hydroxy-4-((6-methyl-5- | + | | | |
| | (methylthiomethyl)pyridin-2-yl)methylamino)-1- | | | | |
| | phenylbutan-2-yl)-N3-methyl-N3-((4- | | | | |
| | methylthiazol-2-yl)methyl)isophthalamide | | | | |
| 88 | N1-((2S,3R)-3-hydroxy-4-(3-iodobenzylamino)-1- | +++ | +++ | +++ | +++ |
| | phenylbutan-2-yl)-5-(N- | | | | |
| | methylmethylsulfonamido)-N3-((R)-1- | | | | |
| | phenylethyl)isophthalamide | | | | |
| 89 | N1-((2S,3R)-4-((5-(2-fluoropropan-2-yl)pyridin-3- | +++ | +++ | +++ | +++ |
| | yl)methylamino)-3-hydroxy-1-phenylbutan-2-yl)- | | | | |
| | 5-(N-methylmethylsulfonamido)-N3-((R)-1- | | | | |
| | phenylethyl)isophthalamide | | | | |
| 90 | N1-((2S,3R)-4-(3-(dimethylamino)-5- | +++ | +++ | + | +++ |
| | isopropylbenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 91 | N1-((2S,3R)-3-hydroxy-4-(3- | + | + | +++ | |
| | methoxybenzylamino)-1-phenylbutan-2-yl)-N3- | | | | |
| | ((R)-1-phenylethyl)benzene-1,3,5-tricarboxamide | | | | |
| 92 | N1-((2S,3R)-4-((5-(2-fluoropropan-2-yl)pyridin-3- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 93 | N1-((2S,3R)-3-hydroxy-4-((5-isopropyl-2,6- | ++ | | | +++ |
| | dimethylpyridin-3-yl)methylamino)-1- | | | | |
| | phenylbutan-2-yl)-N3-((4-methylthiazol-2- | | | | |
| | yl)methyl)isophthalamide | | | | |
| 94 | N1-((2S,3R)-3-hydroxy-4-(3- | + | | | ++ |
| | methoxybenzylamino)-1-phenylbutan-2-yl)-N3- | | | | |
| | methyl-N5-((R)-1-phenylethyl)benzene-1,3,5- | | | | |
| | tricarboxamide | | | | |
| 95 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | N3-((4-methyloxazol-2-yl)methyl)isophthalamide | | | | |
| 96 | methyl 3-(((2R,3S)-2-hydroxy-3-(3-(N- | +++ | | | +++ |
| | methylmethylsulfonamido)-5-((R)-1- | | | | |
| | phenylethylcarbamoyl)benzamido)-4- | | | | |
| | phenylbutylamino)methyl)benzoate | | | | |
| 97 | N1-((2S,3R)-3-hydroxy-4-(3-iodobenzylamino)-1- | +++ | | | +++ |
| | phenylbutan-2-yl)-N3-methyl-N3-((4- | | | | |
| | methylthiazol-2-yl)methyl)isophthalamide | | | | |
| 98 | N1-((2S,3R)-3-hydroxy-4-(3- | +++ | | | ++ |
| | methoxybenzylamino)-1-phenylbutan-2-yl)- | | | | |
| | N3,N5-dimethyl-N3-((4-methylthiazol-2- | | | | |
| | yl)methyl)benzene-1,3,5-tricarboxamide | | | | |
| 99 | N¹-((2S,3R)-3-hydroxy-4-((3- | + | | | ++ |
| | methoxybenzyl)(methyl)amino)-1-phenylbutan-2- | | | | |
| | yl)-N³-methyl-N³-((4-methylthiazol-2- | | | | |
| | yl)methyl)isophthalamide | | | | |
| 100 | 3-((2S,3R)-3-hydroxy-4-((5-isopropylpyridin-3- | +++ | | | +++ |
| | yl)methylamino)-1-phenylbutan-2-ylcarbamoyl)-5- | | | | |
| | (methyl((4-methylthiazol-2- | | | | |
| | yl)methyl)carbamoyl)phenyl methanesulfonate | | | | |
| 101 | N1-((2S,3R)-3-hydroxy-4-((6-methyl-5- | + | | | |
| | (methylsulfonylmethyl)pyridin-2- | | | | |
| | yl)methylamino)-1-phenylbutan-2-yl)-N3-((4- | | | | |
| | methylthiazol-2-yl)methyl)isophthalamide | | | | |
| 102 | N1-((2S,3R)-3-hydroxy-4-(3- | + | | | + |
| | methoxybenzylamino)-1-phenylbutan-2-yl)-N3- | | | | |
| | ((4-methylpyrimidin-2-yl)methyl)isophthalamide | | | | |
| 103 | N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2- | | | | |
| | yl)methyl)benzene-1,3,5-tricarboxamide | | | | |
| 104 | N1-((2S,3R)-4-(3-chlorobenzylamino)-3-hydroxy- | +++ | | | +++ |
| | 1-phenylbutan-2-yl)-N3-methyl-N3-((4- | | | | |
| | methylthiazol-2-yl)methyl)isophthalamide | | | | |
| 105 | N1-((2S,3R)-3-hydroxy-4-(3- | + | | | |
| | methoxybenzylamino)-1-phenylbutan-2-yl)-5-(N- | | | | |
| | methylmethylsulfonamido)-N3-((4- | | | | |
| | methylpyrimidin-2-yl)methyl)isophthalamide | | | | |
| 106 | N1-((2S,3R)-4-((2,6-diisopropylpyridin-4- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 107 | N1-((2S,3R)-3-hydroxy-4-(3- | +++ | | | +++ |
| | (hydroxymethyl)benzylamino)-1-phenylbutan-2- | | | | |
| | yl)-5-(N-methylmethylsulfonamido)-N3-((R)-1- | | | | |
| | phenylethyl)isophthalamide | | | | |
| 108 | N1-((2S,3R)-3-hydroxy-4-(3- | ++ | | | |
| | methoxybenzylamino)-1-phenylbutan-2-yl)-N3- | | | | |
| | methyl-N3-((6-methylpyridin-2- | | | | |
| | yl)methyl)isophthalamide | | | | |
| 109 | N1-((2S,3R)-4-(5-tert-butyl-2-(tert- | +++ | | | |
| | butyldimethylsilyloxy)benzylamino)-3-hydroxy-1- | | | | |
| | phenylbutan-2-yl)-5-(N- | | | | |
| | methylmethylsulfonamido)-N3-((R)-1- | | | | |
| | phenylethyl)isophthalamide | | | | |
| 110 | N1-((2S,3R)-4-(5-tert-butyl-2- | +++ | | | +++ |
| | hydroxybenzylamino)-3-hydroxy-1-phenylbutan- | | | | |
| | 2-yl)-5-(N-methylmethylsulfonamido)-N3-((R)-1- | | | | |
| | phenylethyl)isophthalamide | | | | |
| 111 | N1-((2S,3R)-4-(5-tert-butyl-2- | +++ | | | +++ |
| | hydroxybenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 112 | N1-((2S,3R)-3-hydroxy-4-(3-methoxy-5- | +++ | | | +++ |
| | (methylcarbamoyl)benzylamino)-1-phenylbutan-2- | | | | |
| | yl)-N3-methyl-N3-((4-methylthiazol-2- | | | | |
| | yl)methyl)isophthalamide | | | | |
| 113 | 3-(((2R,3S)-2-hydroxy-3-(3-(N- | +++ | | | +++ |
| | methylmethylsulfonamido)-5-((R)-1- | | | | |
| | phenylethylcarbamoyl)benzamido)-4- | | | | |
| | phenylbutylamino)methyl)benzoic acid | | | | |
| 114 | methyl 3-(((2R,3S)-2-hydroxy-3-(3-(N- | +++ | | | +++ |
| | methylmethylsulfonamido)-5-((R)-1- | | | | |
| | phenylethylcarbamoyl)benzamido)-4- | | | | |
| | phenylbutylamino)methyl)-5-methoxybenzoate | | | | |
| 115 | N1-((2S,3R)-3-hydroxy-4-((6-methylpyridin-2- | + | | | +++ |
| | yl)methylamino)-1-phenylbutan-2-yl)-N3-methyl- | | | | |
| | N3-((4-methylthiazol-2-yl)methyl)isophthalamide | | | | |
| 116 | N1-((2S,3R)-4-(3-(benzyloxy)-5- | +++ | | | +++ |
| | isopropylbenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 117 | N1-((2S,3R)-3-hydroxy-4-(3- | +++ | | | +++ |
| | methoxybenzylamino)-1-phenylbutan-2-yl)-5- | | | | |
| | (hydroxyamino)-N3-methyl-N3-((4-methylthiazol- | | | | |
| | 2-yl)methyl)isophthalamide | | | | |
| 118 | N1-((2S,3R)-4-((1-tert-butyl-5-methyl-1H- | + | | | ++ |
| | pyrazol-4-yl)methylamino)-3-hydroxy-1- | | | | |
| | phenylbutan-2-yl)-N3-methyl-N3-((4- | | | | |
| | methylthiazol-2-yl)methyl)isophthalamide | | | | |
| 119 | N1-((2S,3R)-3-hydroxy-4-(3-isopropyl-5- | +++ | | | +++ |
| | (methylsulfonamido)benzylamino)-1-phenylbutan- | | | | |
| | 2-yl)-N3-methyl-N3-((4-methylthiazol-2- | | | | |
| | yl)methyl)isophthalamide | | | | |
| 120 | N1-((2S,3R)-4-((1-tert-butyl-5-methyl-1H- | ++ | | | |
| | pyrazol-4-yl)methylamino)-3-hydroxy-1- | | | | |
| | phenylbutan-2-yl)-5-(N- | | | | |
| | methylmethylsulfonamido)-N3-((R)-1- | | | | |
| | phenylethyl)isophthalamide | | | | |
| 121 | 5-(azidomethyl)-N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2- | | | | |
| | yl)methyl)isophthalamide | | | | |
| 122 | N1-((2S,3R)-3-hydroxy-4-(3- | +++ | | | +++ |
| | methoxybenzylamino)-1-phenylbutan-2-yl)-5- | | | | |
| | (methoxymethyl)-N3-methyl-N3-((4- | | | | |
| | methylthiazol-2-yl)methyl)isophthalamide | | | | |
| 123 | N1-((2S,3R)-3-hydroxy-4-(3- | + | | | |
| | methoxybenzylamino)-1-phenylbutan-2-yl)-5- | | | | |
| | (hydroxyamino)-N3-((R)-1- | | | | |
| | phenylethyl)isophthalamide | | | | |
| 124 | N1-((2S,3R)-4-(3-acetylbenzylamino)-3-hydroxy- | +++ | | | +++ |
| | 1-phenylbutan-2-yl)-N3-methyl-N3-((4- | | | | |
| | methylthiazol-2-yl)methyl)isophthalamide | | | | |
| 125 | N1-((2S,3R)-3-hydroxy-4-(3-hydroxy-5- | +++ | | | +++ |
| | isopropylbenzylamino)-1-phenylbutan-2-yl)-N3- | | | | |
| | methyl-N3-((4-methylthiazol-2- | | | | |
| | yl)methyl)isophthalamide | | | | |
| 126 | N1-((2S,3R)-4-(3-acetylbenzylamino)-3-hydroxy- | +++ | | | +++ |
| | 1-phenylbutan-2-yl)-5-(N- | | | | |
| | methylmethylsulfonamido)-N3-((R)-1- | | | | |
| | phenylethyl)isophthalamide | | | | |
| 127 | N1-((2S,3R)-3-hydroxy-4-(3- | + | | | |
| | methoxybenzylamino)-1-phenylbutan-2-yl)-5-(N- | | | | |
| | methylmethylsulfonamido)-N3-((6-methylpyridin- | | | | |
| | 2-yl)methyl)isophthalamide | | | | |
| 128 | N1-((2R,3S)-3-hydroxy-4-((5-isopropylpyridin-3- | + | | | |
| | yl)methylamino)-1-phenylbutan-2-yl)-N3-methyl- | | | | |
| | N3-((4-methylthiazol-2-yl)methyl)isophthalamide | | | | |
| 129 | N1-((2S,3R)-4-(3-(dimethylamino)benzylamino)- | +++ | | | +++ |
| | 3-hydroxy-1-phenylbutan-2-yl)-N3-methyl-N3- | | | | |
| | ((4-methylthiazol-2-yl)methyl)isophthalamide | | | | |
| 130 | benzyl 3-(((2R,3S)-2-hydroxy-3-(3-(methyl((4- | ++ | | | + |
| | methylthiazol-2-yl)methyl)carbamoyl)benzamido)- | | | | |
| | 4-phenylbutylamino)methyl)-5- | | | | |
| | isopropylphenyl(methyl)carbamate | | | | |
| 131 | N1-((2S,3R)-3-hydroxy-4-((1-(2-methoxyethyl)-5- | +++ | | | |
| | methyl-1H-pyrazol-4-yl)methylamino)-1- | | | | |
| | phenylbutan-2-yl)-5-(N- | | | | |
| | methylmethylsulfonamido)-N3-((R)-1- | | | | |
| | phenylethyl)isophthalamide | | | | |
| 132 | N1-((2S,3R)-3-hydroxy-4-((1-(2-methoxyethyl)-5- | + | | | |
| | methyl-1H-pyrazol-4-yl)methylamino)-1- | | | | |
| | phenylbutan-2-yl)-N3-methyl-N3-((4- | | | | |
| | methylthiazol-2-yl)methyl)isophthalamide | | | | |
| 133 | N1-((2S,3R)-3-hydroxy-4-(3-isopropyl-5- | +++ | | | +++ |
| | (methylamino)benzylamino)-1-phenylbutan-2-yl)- | | | | |
| | N3-methyl-N3-((4-methylthiazol-2- | | | | |
| | yl)methyl)isophthalamide | | | | |
| 134 | methyl 3-(((2R,3S)-2-hydroxy-3-(3-(methyl((4- | +++ | | | ++ |
| | methylthiazol-2-yl)methyl)carbamoyl)benzamido)- | | | | |
| | 4-phenylbutylamino)methyl)-5-(N- | | | | |
| | methylmethylsulfonamido)benzoate | | | | |
| 135 | methyl 3-(((2R,3S)-2-hydroxy-3-(3-(methyl((4- | +++ | | | ++ |
| | methylthiazol-2-yl)methyl)carbamoyl)benzamido)- | | | | |
| | 4-phenylbutylamino)methyl)-5-(N- | | | | |
| | methylmethylsulfonamido)benzoate | | | | |
| 136 | N1-((2S,3R)-3-hydroxy-4-(3- | + | | | |
| | methoxybenzylamino)-1-phenylbutan-2-yl)-N3- | | | | |
| | methyl-N3-((4-methylpyrimidin-2- | | | | |
| | yl)methyl)isophthalamide | | | | |
| 137 | N1-((2S,3R)-4-(3-cyanobenzylamino)-3-hydroxy- | +++ | | | +++ |
| | 1-phenylbutan-2-yl)-N3-methyl-N3-((4- | | | | |
| | methylthiazol-2-yl)methyl)isophthalamide | | | | |
| 138 | N1-((2S,3R)-4-((1-tert-butyl-1H-pyrazol-4- | + | | | |
| | yl)methylamino)-3-hydroxy-1-phenylbutan-2-yl)- | | | | |
| | 5-(N-methylmethylsulfonamido)-N3-((R)-1- | | | | |
| | phenylethyl)isophthalamide | | | | |
| 139 | 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5-(N- | | | | |
| | methylmethylsulfonamido)benzoic acid | | | | |
| 140 | 2′-cyano-N3-((2S,3R)-3-hydroxy-4-(3- | +++ | | | +++ |
| | methoxybenzylamino)-1-phenylbutan-2-yl)-N5- | | | | |
| | methyl-N5-((4-methylthiazol-2- | | | | |
| | yl)methyl)biphenyl-3,5-dicarboxamide | | | | |
| 141 | methyl 3-(((2R,3S)-2-hydroxy-3-(3-(methyl((4- | ++ | | | +++ |
| | methylthiazol-2-yl)methyl)carbamoyl)benzamido)- | | | | |
| | 4-phenylbutylamino)methyl)benzoate | | | | |
| 142 | N1-((2S,3R)-4-(3-(difluoromethyl)benzylamino)- | +++ | | | +++ |
| | 3-hydroxy-1-phenylbutan-2-yl)-5-(N- | | | | |
| | methylmethylsulfonamido)-N3-((R)-1- | | | | |
| | phenylethyl)isophthalamide | | | | |
| 143 | N1-((2S,3R)-4-(3-(difluoromethyl)benzylamino)- | +++ | | | +++ |
| | 3-hydroxy-1-phenylbutan-2-yl)-N3-methyl-N3- | | | | |
| | ((4-methylthiazol-2-yl)methyl)isophthalamide | | | | |
| 144 | N1-((2S,3R)-4-(3-(1,1- | +++ | | | +++ |
| | difluoroethyl)benzylamino)-3-hydroxy-1- | | | | |
| | phenylbutan-2-yl)-5-(N- | | | | |
| | methylmethylsulfonamido)-N3-((R)-1- | | | | |
| | phenylethyl)isophthalamide | | | | |
| 145 | N1-((2S,3R)-4-(3-(1,1- | +++ | | | +++ |
| | difluoroethyl)benzylamino)-3-hydroxy-1- | | | | |
| | phenylbutan-2-yl)-N3-methyl-N3-((4- | | | | |
| | methylthiazol-2-yl)methyl)isophthalamide | | | | |
| 146 | N1-((2S,3R)-3-hydroxy-4-(3-isopropyl-5-(N- | +++ | | | +++ |
| | methylmethylsulfonamido)benzylamino)-1- | | | | |
| | phenylbutan-2-yl)-N3-methyl-N3-((4- | | | | |
| | methylthiazol-2-yl)methyl)isophthalamide | | | | |
| 147 | N1-((2S,3S)-3-hydroxy-4-((5-isopropylpyridin-3- | + | | | |
| | yl)methylamino)-1-phenylbutan-2-yl)-N3-methyl- | | | | |
| | N3-((4-methylthiazol-2-yl)methyl)isophthalamide | | | | |
| 148 | N1-((2S,3R)-3-hydroxy-4-(1-(5-isopropylpyridin- | +++ | | | +++ |
| | 3-yl)ethylamino)-1-phenylbutan-2-yl)-N3-methyl- | | | | |
| | N3-((4-methylthiazol-2-yl)methyl)isophthalamide | | | | |
| 149 | N1-((2S,3R)-4-(3-cyanobenzylamino)-3-hydroxy- | +++ | | | +++ |
| | 1-phenylbutan-2-yl)-5-(N- | | | | |
| | methylmethylsulfonamido)-N3-((R)-1- | | | | |
| | phenylethyl)isophthalamide | | | | |
| 150 | N1-((2S,3R)-3-hydroxy-4-(3-isopropyl-5- | +++ | | | +++ |
| | (methylsulfonyl)benzylamino)-1-phenylbutan-2- | | | | |
| | yl)-N3-methyl-N3-((4-methylthiazol-2- | | | | |
| | yl)methyl)isophthalamide | | | | |
| 151 | N1-((2R,3R)-3-hydroxy-4-((5-isopropylpyridin-3- | + | | | |
| | yl)methylamino)-1-phenylbutan-2-yl)-N3-methyl- | | | | |
| | N3-((4-methylthiazol-2-yl)methyl)isophthalamide | | | | |
| 152 | N1-((2S,3R)-4-(3-(diethylamino)benzylamino)-3- | +++ | | | |
| | hydroxy-1-phenylbutan-2-yl)-N3-methyl-N3-((4- | | | | |
| | methylthiazol-2-yl)methyl)isophthalamide | | | | |
| 153 | N1-((2S,3R)-3-hydroxy-4-((5-isopropyl-2- | +++ | | | |
| | methylpyridin-3-yl)methylamino)-1-phenylbutan- | | | | |
| | 2-yl)-N3-methyl-N3-((4-methylthiazol-2- | | | | |
| | yl)methyl)isophthalamide | | | | |
| 154 | 2′-cyano-N3-((2S,3R)-3-hydroxy-4-((5- | +++ | | | +++ |
| | isopropylpyridin-3-yl)methylamino)-1- | | | | |
| | phenylbutan-2-yl)-N5-methyl-N5-((4- | | | | |
| | methylthiazol-2-yl)methyl)biphenyl-3,5- | | | | |
| | dicarboxamide | | | | |
| 155 | N1-((2S,3R)-4-((2-amino-6-isopropylpyrimidin-4- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 156 | N1-((2S,3R)-4-(bis((1-methyl-1H-pyrazol-4- | + | | | |
| | yl)methyl)amino)-3-hydroxy-1-phenylbutan-2-yl)- | | | | |
| | N3-methyl-N3-((4-methylthiazol-2- | | | | |
| | yl)methyl)isophthalamide | | | | |
| 157 | N1-((2S,3R)-4-(1-(3- | +++ | | | |
| | (difluoromethyl)phenyl)ethylamino)-3-hydroxy-1- | | | | |
| | phenylbutan-2-yl)-N3-methyl-N3-((4- | | | | |
| | methylthiazol-2-yl)methyl)isophthalamide | | | | |
| 158 | N1-((2S,3R)-4-((5-bromopyridin-3- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 159 | N1-((2S,3R)-3-hydroxy-4-(3-hydroxy-5-(prop-1- | +++ | | | |
| | en-2-yl)benzylamino)-1-phenylbutan-2-yl)-N3- | | | | |
| | methyl-N3-((4-methylthiazol-2- | | | | |
| | yl)methyl)isophthalamide | | | | |
| 160 | N1-((2S,3R)-3-hydroxy-4-(3-(1- | +++ | | | |
| | hydroxyethyl)benzylamino)-1-phenylbutan-2-yl)- | | | | |
| | N3-methyl-N3-((4-methylthiazol-2- | | | | |
| | yl)methyl)isophthalamide | | | | |
| 161 | N1-((2S,3R)-3-hydroxy-4-((6-isopropyl-2- | +++ | | | +++ |
| | (methylamino)pyrimidin-4-yl)methylamino)-1- | | | | |
| | phenylbutan-2-yl)-N3-methyl-N3-((4- | | | | |
| | methylthiazol-2-yl)methyl)isophthalamide | | | | |
| 162 | 5-(dimethylamino)-N1-((2S,3R)-3-hydroxy-4-((5- | +++ | | | +++ |
| | isopropylpyridin-3-yl)methylamino)-1- | | | | |
| | phenylbutan-2-yl)-N3-methyl-N3-((4- | | | | |
| | methylthiazol-2-yl)methyl)isophthalamide | | | | |
| 163 | N1-((2S,3R)-3-hydroxy-4-(2-(3- | +++ | | | +++ |
| | methoxyphenyl)propan-2-ylamino)-1- | | | | |
| | phenylbutan-2-yl)-N3-methyl-N3-((4- | | | | |
| | methylthiazol-2-yl)methyl)isophthalamide | | | | |
| 164 | N1-((2S,3R)-4-(3-(benzylamino)benzylamino)-3- | +++ | | | +++ |
| | hydroxy-1-phenylbutan-2-yl)-N3-methyl-N3-((4- | | | | |
| | methylthiazol-2-yl)methyl)isophthalamide | | | | |
| 165 | 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5-isopropylphenyl | | | | |
| | methanesulfonate | | | | |
| 166 | N1-((2S,3R)-3-hydroxy-4-(3- | + | | | + |
| | methoxyphenylamino)-1-phenylbutan-2-yl)-N3- | | | | |
| | methyl-N3-((4-methylthiazol-2- | | | | |
| | yl)methyl)isophthalamide | | | | |
| 167 | N1-((2S,3R)-3-hydroxy-4-(3- | +++ | | | +++ |
| | (methylamino)benzylamino)-1-phenylbutan-2-yl)- | | | | |
| | N3-methyl-N3-((4-methylthiazol-2- | | | | |
| | yl)methyl)isophthalamide | | | | |
| 168 | N1-((2S,3R)-3-hydroxy-1-phenyl-4-(3-(prop-1-en- | +++ | | | +++ |
| | 2-yl)benzylamino)butan-2-yl)-N3-methyl-N3-((4- | | | | |
| | methylthiazol-2-yl)methyl)isophthalamide | | | | |
| 169 | 5-(diethylamino)-N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2- | | | | |
| | yl)methyl)isophthalamide | | | | |
| 170 | N1-((2S,3R)-3-hydroxy-4-((5-isopropylpyridin-3- | +++ | | +++ | +++ |
| | yl)methylamino)-1-phenylbutan-2-yl)-N3-methyl- | | | | |
| | N3-((4-methylthiazol-2-yl)methyl)-5-(oxazol-2- | | | | |
| | yl)isophthalamide | | | | |
| 171 | N1-((2S,3R)-4-((5-cyanopyridin-3- | ++ | | | ++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 172 | 5-(diethylamino)-N1-((2S,3R)-3-hydroxy-4-((5- | +++ | | | +++ |
| | isopropylpyridin-3-yl)methylamino)-1- | | | | |
| | phenylbutan-2-yl)-N3-methyl-N3-((4- | | | | |
| | methylthiazol-2-yl)methyl)isophthalamide | | | | |
| 173 | N1-((2S,3R)-4-(3-(1-chloroethyl)benzylamino)-3- | +++ | | | +++ |
| | hydroxy-1-phenylbutan-2-yl)-N3-methyl-N3-((4- | | | | |
| | methylthiazol-2-yl)methyl)isophthalamide | | | | |
| 174 | N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2-yl)methyl)-5- | | | | |
| | (pyridin-2-yl)isophthalamide | | | | |
| 175 | N1-((2S,3R)-3-hydroxy-4-(3-isopropyl-5- | + | | | + |
| | (methylsulfonamidomethyl)benzylamino)-1- | | | | |
| | phenylbutan-2-yl)-N3-methyl-N3-((4- | | | | |
| | methylthiazol-2-yl)methyl)isophthalamide | | | | |
| 176 | 5-(fluoromethyl)-N1-((2S,3R)-3-hydroxy-4-((5- | +++ | | | +++ |
| | isopropylpyridin-3-yl)methylamino)-1- | | | | |
| | phenylbutan-2-yl)-N3-methyl-N3-((4- | | | | |
| | methylthiazol-2-yl)methyl)isophthalamide | | | | |
| 177 | N1-((2S,3R)-4-((2-amino-6-isopropylpyrimidin-4- | +++ | | | +++ |
| | yl)methylamino)-3-hydroxy-1-phenylbutan-2-yl)- | | | | |
| | N3-methyl-N3-((4-methylthiazol-2-yl)methyl)-5- | | | | |
| | (oxazol-2-yl)isophthalamide | | | | |
| 178 | 3-(((2R,3S)-3-(3-(dimethylamino)-5-(methyl((4- | +++ | | | +++ |
| | methylthiazol-2-yl)methyl)carbamoyl)benzamido)- | | | | |
| | 2-hydroxy-4-phenylbutylamino)methyl)-5- | | | | |
| | isopropylphenyl methanesulfonate | | | | |
| 179 | N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2-yl)methyl)-5- | | | | |
| | (pyrrolidin-1-yl)isophthalamide | | | | |
| 180 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | N3-((4-methylthiazol-2-yl)methyl)-5-(pyrrolidin- | | | | |
| | 1-yl)isophthalamide | | | | |
| 181 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | N3-((4-methylthiazol-2-yl)methyl)-5-(pyridin-2- | | | | |
| | yl)isophthalamide | | | | |
| 182 | N1-((2S,3R)-3-hydroxy-4-((S)-1-(5- | +++ | | | +++ |
| | isopropylpyridin-3-yl)ethylamino)-1-phenylbutan- | | | | |
| | 2-yl)-N3-methyl-N3-((4-methylthiazol-2- | | | | |
| | yl)methyl)isophthalamide | | | | |
| 183 | N1-((2S,3R)-3-hydroxy-4-((5-isopropylpyridin-3- | ++ | | | ++ |
| | yl)methylamino)-1-(thiazol-4-yl)butan-2-yl)-N3- | | | | |
| | methyl-N3-((4-methylthiazol-2- | | | | |
| | yl)methyl)isophthalamide | | | | |
| 184 | N1-((2S,3R)-3-hydroxy-4-(3-(2-hydroxypropan-2- | +++ | | | +++ |
| | yl)benzylamino)-1-phenylbutan-2-yl)-N3-methyl- | | | | |
| | N3-((4-methylthiazol-2-yl)methyl)isophthalamide | | | | |
| 185 | N1-((2S,3R)-4-(3,5- | +++ | | | +++ |
| | bis(dimethylamino)benzylamino)-3-hydroxy-1- | | | | |
| | phenylbutan-2-yl)-N3-methyl-N3-((4- | | | | |
| | methylthiazol-2-yl)methyl)isophthalamide | | | | |
| 186 | N1-cyclopropyl-N3-((2S,3R)-3-hydroxy-4-((5- | +++ | | | +++ |
| | isopropylpyridin-3-yl)methylamino)-1- | | | | |
| | phenylbutan-2-yl)-N1-((4-methylthiazol-2- | | | | |
| | yl)methyl)-5-(oxazol-2-yl)isophthalamide | | | | |
| 187 | N1-((2S,3R)-3-hydroxy-4-(3- | +++ | | | + |
| | methoxybenzylamino)-1-phenylbutan-2-yl)-N3- | | | | |
| | ((4-methylthiazol-2-yl)methyl)-5-(pyrrolidin-1- | | | | |
| | yl)isophthalamide | | | | |
| 188 | N1-((2S,3R)-4-(3-(ethylsulfonyl)-5- | +++ | | | +++ |
| | isopropylbenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 189 | N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2-yl)methyl)-5- | | | | |
| | (pyridin-3-yl)isophthalamide | | | | |
| 190 | N1-((2S,3R)-4-((5-tert-butylpyridin-3- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 191 | N1-((2S,3R)-4-(3-aminobenzylamino)-3-hydroxy- | + | | | + |
| | 1-phenylbutan-2-yl)-N3-methyl-N3-((4- | | | | |
| | methylthiazol-2-yl)methyl)isophthalamide | | | | |
| 192 | N1-((2S,3R)-3-hydroxy-4-((5-isopropylpyridin-3- | +++ | | | +++ |
| | yl)methylamino)-1-phenylbutan-2-yl)-5- | | | | |
| | (hydroxymethyl)-N3-methyl-N3-((4- | | | | |
| | methylthiazol-2-yl)methyl)isophthalamide | | | | |
| 193 | N1-((2S,3R)-4-(3-acetamido-5- | +++ | | | +++ |
| | isopropylbenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 194 | N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2-yl)methyl)-5-(2- | | | | |
| | oxopyrrolidin-1-yl)isophthalamide | | | | |
| 195 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | N3-((4-methylthiazol-2-yl)methyl)-5-(2- | | | | |
| | oxopyrrolidin-1-yl)isophthalamide | | | | |
| 196 | N1-((2S,3R)-3-hydroxy-1-phenyl-4-((5- | +++ | | | +++ |
| | phenylisoxazol-3-yl)methylamino)butan-2-yl)-N3- | | | | |
| | methyl-N3-((4-methylthiazol-2- | | | | |
| | yl)methyl)isophthalamide | | | | |
| 197 | N1-((2S,3R)-3-hydroxy-4-((5-isopropylpyridin-3- | +++ | | | +++ |
| | yl)methylamino)-1-phenylbutan-2-yl)-N3-((4- | | | | |
| | methylthiazol-2-yl)methyl)-5-(oxazol-2- | | | | |
| | yl)isophthalamide | | | | |
| 198 | 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5-isopropylphenyl | | | | |
| | dimethylcarbamate | | | | |
| 199 | N1-((2S,3R)-3-hydroxy-4-(3- | + | | | + |
| | methoxybenzylamino)-1-phenylbutan-2-yl)-N3- | | | | |
| | ((4-methylthiazol-2-yl)methyl)-5-(2- | | | | |
| | oxopyrrolidin-1-yl)isophthalamide | | | | |
| 200 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | N3-((4-methylthiazol-2-yl)methyl)-5-(pyridin-3- | | | | |
| | yl)isophthalamide | | | | |
| 201 | N1-((2S,3R)-3-hydroxy-4-((R)-1-(3- | +++ | | | +++ |
| | methoxyphenyl)ethylamino)-1-phenylbutan-2-yl)- | | | | |
| | 5-(N-methylmethylsulfonamido)-N3-((R)-1- | | | | |
| | phenylethyl)isophthalamide | | | | |
| 202 | methyl 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5- | | | | |
| | isopropylphenylcarbamate | | | | |
| 203 | N1-((2S,3R)-3-hydroxy-4-(3- | +++ | | | |
| | methoxybenzylamino)-1-phenylbutan-2-yl)-N3- | | | | |
| | methyl-N3-((4-methylthiazol-2-yl)methyl)-5- | | | | |
| | (pyridin-4-yl)isophthalamide | | | | |
| 204 | 5-(difluoromethyl)-N1-((2S,3R)-3-hydroxy-4-((5- | +++ | | | +++ |
| | isopropylpyridin-3-yl)methylamino)-1- | | | | |
| | phenylbutan-2-yl)-N3-methyl-N3-((4- | | | | |
| | methylthiazol-2-yl)methyl)isophthalamide | | | | |
| 205 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | N3-((4-methylthiazol-2-yl)methyl)-5-(pyridin-4- | | | | |
| | yl)isophthalamide | | | | |
| 206 | N1-((2S,3R)-3-hydroxy-4-((R)-1-(5- | +++ | | | +++ |
| | isopropylpyridin-3-yl)ethylamino)-1-phenylbutan- | | | | |
| | 2-yl)-N3-methyl-N3-((4-methylthiazol-2- | | | | |
| | yl)methyl)isophthalamide | | | | |
| 207 | N1-((2S,3R)-4-((2-amino-6-methoxypyrimidin-4- | ++ | | | + |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 208 | N1-((2S,3R)-1-(3,5-difluorophenyl)-3-hydroxy-4- | +++ | | +++ | +++ |
| | ((5-isopropylpyridin-3-yl)methylamino)butan-2- | | | | |
| | yl)-N3-methyl-N3-((4-methylthiazol-2- | | | | |
| | yl)methyl)isophthalamide | | | | |
| 209 | 3-(((2R,3S)-3-(3-(dimethylamino)-5-(methyl((4- | +++ | | | +++ |
| | methylthiazol-2-yl)methyl)carbamoyl)benzamido)- | | | | |
| | 2-hydroxy-4-phenylbutylamino)methyl)-5- | | | | |
| | isopropylphenyl dimethylcarbamate | | | | |
| 210 | N3-((2S,3R)-3-hydroxy-4-(3- | +++ | | | +++ |
| | methoxybenzylamino)-1-phenylbutan-2-yl)-2′- | | | | |
| | methoxy-N5-methyl-N5-((4-methylthiazol-2- | | | | |
| | yl)methyl)biphenyl-3,5-dicarboxamide | | | | |
| 211 | N1-((2S,3R)-3-hydroxy-4-((5-isopropylpyridin-3- | +++ | | | +++ |
| | yl)methylamino)-1-phenylbutan-2-yl)-5-(oxazol-2- | | | | |
| | yl)-N3-((R)-1-phenylethyl)isophthalamide | | | | |
| 212 | N1-((2S,3R)-4-((2-(dimethylamino)-6- | +++ | | | +++ |
| | methoxypyrimidin-4-yl)methylamino)-3-hydroxy- | | | | |
| | 1-phenylbutan-2-yl)-N3-methyl-N3-((4- | | | | |
| | methylthiazol-2-yl)methyl)isophthalamide | | | | |
| 213 | N1-((2S,3R)-3-hydroxy-4-(3- | ++ | | | +++ |
| | methoxybenzylamino)-1-phenylbutan-2-yl)-5- | | | | |
| | (oxazol-2-yl)-N3-((R)-1- | | | | |
| | phenylethyl)isophthalamide | | | | |
| 214 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | N3-((4-methylthiazol-2-yl)methyl)-5-(pyrazin-2- | | | | |
| | yl)isophthalamide | | | | |
| 215 | N1-((2S,3R)-4-(3-tert-butyl-5- | +++ | | | +++ |
| | hydroxybenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 216 | N1-((2S,3R)-4-((5-chloropyridin-3- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 217 | N1-((2S,3R)-4-((5-(1,1-difluoroethyl)pyridin-3- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 218 | N1-((2S,3R)-3-hydroxy-4-((5-isopropylpyridin-3- | +++ | | +++ | +++ |
| | yl)methylamino)-1-phenylbutan-2-yl)-N3-methyl- | | | | |
| | N3-((4-methylthiazol-2-yl)methyl)-5-(oxazol-5- | | | | |
| | yl)isophthalamide | | | | |
| 219 | N1-((2S,3R)-3-hydroxy-1-phenyl-4-(3- | +++ | | | |
| | (trifluoromethyl)benzylamino)butan-2-yl)-N3- | | | | |
| | methyl-N3-((4-methylthiazol-2-yl)methyl)-5- | | | | |
| | (oxazol-5-yl)isophthalamide | | | | |
| 220 | 5-(dimethylamino)-N1-((2S,3R)-4-((5-(2- | +++ | | +++ | +++ |
| | fluoropropan-2-yl)pyridin-3-yl)methylamino)-3- | | | | |
| | hydroxy-1-phenylbutan-2-yl)-N3-methyl-N3-((4- | | | | |
| | methylthiazol-2-yl)methyl)isophthalamide | | | | |
| 221 | N1-((2S,3R)-3-hydroxy-4-(3-methoxy-5- | +++ | | | +++ |
| | nitrobenzylamino)-1-phenylbutan-2-yl)-N3- | | | | |
| | methyl-N3-((4-methylthiazol-2- | | | | |
| | yl)methyl)isophthalamide | | | | |
| 222 | N1-((2S,3R)-3-hydroxy-1-phenyl-4-(3- | +++ | | | +++ |
| | (trifluoromethyl)benzylamino)butan-2-yl)-N3- | | | | |
| | methyl-N3-((4-methylthiazol-2-yl)methyl)-5- | | | | |
| | (pyrazin-2-yl)isophthalamide | | | | |
| 223 | N1-((2S,3R)-3-hydroxy-4-((5-isopropylpyridin-3- | +++ | | | +++ |
| | yl)methylamino)-1-phenylbutan-2-yl)-5-(1- | | | | |
| | hydroxyethyl)-N3-methyl-N3-((4-methylthiazol-2- | | | | |
| | yl)methyl)isophthalamide | | | | |
| 224 | N1-((2S,3R)-4-(3-(dimethylamino)-5- | +++ | | | +++ |
| | methoxybenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 225 | N1-((2S,3R)-4-((5-fluoropyridin-3- | +++ | | | ++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 226 | N1-((2S,3R)-4-((5-tert-butylpyridin-3- | +++ | | | +++ |
| | yl)methylamino)-3-hydroxy-1-phenylbutan-2-yl)- | | | | |
| | 5-(dimethylamino)-N3-methyl-N3-((4- | | | | |
| | methylthiazol-2-yl)methyl)isophthalamide | | | | |
| 227 | N1-((2S,3R)-4-(3-(1,1- | +++ | | | +++ |
| | difluoroethyl)benzylamino)-3-hydroxy-1- | | | | |
| | phenylbutan-2-yl)-5-(dimethylamino)-N3-methyl- | | | | |
| | N3-((4-methylthiazol-2-yl)methyl)isophthalamide | | | | |
| 228 | N1-((2S,3R)-3-hydroxy-4-(3-isopropyl-5- | +++ | | ++ | +++ |
| | (methylsulfonylmethyl)benzylamino)-1- | | | | |
| | phenylbutan-2-yl)-N3-methyl-N3-((4- | | | | |
| | methylthiazol-2-yl)methyl)isophthalamide | | | | |
| 229 | N1-((2S,3R)-3-hydroxy-4-(3-isopropyl-5- | +++ | | | + |
| | (isopropylsulfonyl)benzylamino)-1-phenylbutan-2- | | | | |
| | yl)-N3-methyl-N3-((4-methylthiazol-2- | | | | |
| | yl)methyl)isophthalamide | | | | |
| 230 | N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2-yl)methyl)-5- | | | | |
| | (piperidin-1-yl)isophthalamide | | | | |
| 231 | N1-((2S,3R)-3-hydroxy-4-((5-isopropylpyridin-3- | +++ | | | + |
| | yl)methylamino)-1-phenylbutan-2-yl)-N3-methyl- | | | | |
| | N3-((4-methylthiazol-2-yl)methyl)-5-(piperidin-1- | | | | |
| | yl)isophthalamide | | | | |
| 232 | N1-((2S,3R)-3-hydroxy-1-phenyl-4-(3- | +++ | | | +++ |
| | (trifluoromethyl)benzylamino)butan-2-yl)-N3,5- | | | | |
| | dimethyl-N3-((4-methylthiazol-2- | | | | |
| | yl)methyl)isophthalamide | | | | |
| 233 | N1-((2S,3R)-3-hydroxy-1-phenyl-4-(3- | +++ | | | +++ |
| | (trifluoromethyl)benzylamino)butan-2-yl)-N3- | | | | |
| | methyl-N3-((4-methylthiazol-2-yl)methyl)-5-(2- | | | | |
| | oxopyrrolidin-1-yl)isophthalamide | | | | |
| 234 | 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5-isopropylphenyl | | | | |
| | morpholine-4-carboxylate | | | | |
| 235 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | N3-((3-methyl-1,2,4-oxadiazol-5- | | | | |
| | yl)methyl)isophthalamide | | | | |
| 236 | N1-((2S,3R)-4-(3,5-dichlorobenzylamino)-3- | +++ | | | +++ |
| | hydroxy-1-phenylbutan-2-yl)-N3-methyl-N3-((4- | | | | |
| | methylthiazol-2-yl)methyl)isophthalamide | | | | |
| 237 | 5-acetyl-N1-((2S,3R)-3-hydroxy-4-((5- | +++ | | | +++ |
| | isopropylpyridin-3-yl)methylamino)-1- | | | | |
| | phenylbutan-2-yl)-N3-methyl-N3-((4- | | | | |
| | methylthiazol-2-yl)methyl)isophthalamide | | | | |
| 238 | N1-((2S,3R)-3-hydroxy-4-(3- | +++ | | | +++ |
| | methoxybenzylamino)-1-phenylbutan-2-yl)-N3- | | | | |
| | methyl-N3-((4-methylthiazol-2-yl)methyl)-5-(1H- | | | | |
| | pyrrol-1-yl)isophthalamide | | | | |
| 239 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | N3-((4-methylthiazol-2-yl)methyl)-5-(1H-pyrrol- | | | | |
| | 1-yl)isophthalamide | | | | |
| 240 | N1-((2S,3R)-3-hydroxy-4-((5-isopropylpyridin-3- | ++ | | | ++ |
| | yl)methylamino)-1-phenylbutan-2-yl)-N3-methyl- | | | | |
| | N3-((5-methyl-1,2,4-oxadiazol-3- | | | | |
| | yl)methyl)isophthalamide | | | | |
| 241 | N1-((2S,3R)-3-hydroxy-4-(3- | +++ | | | ++ |
| | methoxybenzylamino)-1-phenylbutan-2-yl)-5-(3- | | | | |
| | hydroxypyrrolidin-1-yl)-N3-methyl-N3-((4- | | | | |
| | methylthiazol-2-yl)methyl)isophthalamide | | | | |
| 242 | N1-((2S,3R)-3-hydroxy-4-((5-isopropylpyridin-3- | +++ | | | +++ |
| | yl)methylamino)-1-phenylbutan-2-yl)-5-(3- | | | | |
| | hydroxypyrrolidin-1-yl)-N3-methyl-N3-((4- | | | | |
| | methylthiazol-2-yl)methyl)isophthalamide | | | | |
| 243 | 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5-isopropylphenyl | | | | |
| | acetate | | | | |
| 244 | 5-acetyl-N1-((2S,3R)-3-hydroxy-1-phenyl-4-(3- | +++ | | | +++ |
| | (trifluoromethyl)benzylamino)butan-2-yl)-N3- | | | | |
| | methyl-N3-((4-methylthiazol-2- | | | | |
| | yl)methyl)isophthalamide | | | | |
| 245 | N1-((2S,3R)-4-((5-(1,1-difluoroethyl)pyridin-3- | +++ | | | +++ |
| | yl)methylamino)-3-hydroxy-1-phenylbutan-2-yl)- | | | | |
| | 5-(difluoromethyl)-N3-methyl-N3-((4- | | | | |
| | methylthiazol-2-yl)methyl)isophthalamide | | | | |
| 246 | 5-(difluoromethyl)-N1-((2S,3R)-3-hydroxy-1- | +++ | | | +++ |
| | phenyl-4-((5-(trifluoromethyl)pyridin-3- | | | | |
| | yl)methylamino)butan-2-yl)-N3-methyl-N3-((4- | | | | |
| | methylthiazol-2-yl)methyl)isophthalamide | | | | |
| 246 | N1-((2S,3R)-3-hydroxy-4-((5-isopropylpyridin-3- | +++ | | | +++ |
| | yl)methylamino)-1-phenylbutan-2-yl)-4-methyl- | | | | |
| | N3-((4-methylthiazol-2-yl)methyl)isophthalamide | | | | |
| 248 | N1-((2S,3R)-4-(3-cyano-5- | +++ | | | +++ |
| | isopropylbenzylamino)-3-hydroxy-1-phenylbutan- | | | | |
| | 2-yl)-N3-methyl-N3-((4-methylthiazol-2- | | | | |
| | yl)methyl)isophthalamide | | | | |
| 249 | N3-((2S,3R)-3-hydroxy-4-((5-isopropylpyridin-3- | + | | | + |
| | yl)methylamino)-1-phenylbutan-2-yl)-N1,4- | | | | |
| | dimethyl-N1-((4-methylthiazol-2- | | | | |
| | yl)methyl)isophthalamide | | | | |
| 250 | N1-((2S,3R)-4-((5-(2-fluoropropan-2-yl)pyridin-3- | +++ | | | +++ |
| | yl)methylamino)-3-hydroxy-1-phenylbutan-2-yl)- | | | | |
| | N3-methyl-N3-((4-methylthiazol-2-yl)methyl)-5- | | | | |
| | (1H-pyrrol-1-yl)isophthalamide | | | | |
| 251 | N1-((2S,3R)-3-hydroxy-4-(3-isopropyl-5-(N- | +++ | | | +++ |
| | methylsulfamoyl)benzylamino)-1-phenylbutan-2- | | | | |
| | yl)-N3-methyl-N3-((4-methylthiazol-2- | | | | |
| | yl)methyl)isophthalamide | | | | |
| 252 | 5-(furan-2-yl)-N1-((2S,3R)-3-hydroxy-4-((5- | +++ | | | +++ |
| | isopropylpyridin-3-yl)methylamino)-1- | | | | |
| | phenylbutan-2-yl)-N3-methyl-N3-((4- | | | | |
| | methylthiazol-2-yl)methyl)isophthalamide | | | | |
| 253 | N1-((2S,3R)-4-((5-(1,1-difluoroethyl)pyridin-3- | +++ | | | +++ |
| | yl)methylamino)-3-hydroxy-1-phenylbutan-2-yl)- | | | | |
| | 5-(dimethylamino)-N3-methyl-N3-((4- | | | | |
| | methylthiazol-2-yl)methyl)isophthalamide | | | | |
| 254 | 5-(dimethylamino)-N1-((2S,3R)-3-hydroxy-1- | +++ | | | +++ |
| | phenyl-4-(3-(trifluoromethyl)benzylamino)butan- | | | | |
| | 2-yl)-N3-methyl-N3-((4-methylthiazol-2- | | | | |
| | yl)methyl)isophthalamide | | | | |
| 255 | 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5-isopropylphenyl | | | | |
| | dimethyl phosphate | | | | |
| 256 | N1-((2S,3R)-3-hydroxy-4-((5-isopropylpyridin-3- | + | | | ++ |
| | yl)methylamino)-1-phenylbutan-2-yl)-N3-methyl- | | | | |
| | N3-((5-methyl-1,3,4-oxadiazol-2- | | | | |
| | yl)methyl)isophthalamide | | | | |
| 257 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | 5-(1-methyl-1H-pyrrol-2-yl)-N3-((4- | | | | |
| | methylthiazol-2-yl)methyl)isophthalamide | | | | |
| 258 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | 5-(1-methyl-1H-pyrazol-4-yl)-N3-((4- | | | | |
| | methylthiazol-2-yl)methyl)isophthalamide | | | | |
| 259 | 5-(6-fluoropyridin-3-yl)-N1-((2S,3R)-3-hydroxy- | +++ | | | +++ |
| | 4-((5-isopropylpyridin-3-yl)methylamino)-1- | | | | |
| | phenylbutan-2-yl)-N3-methyl-N3-((4- | | | | |
| | methylthiazol-2-yl)methyl)isophthalamide | | | | |
| 260 | 5-(dimethylamino)-N1-((2S,3R)-3-hydroxy-4-((S)- | ++ | | | +++ |
| | 1-(3-methoxyphenyl)ethylamino)-1-phenylbutan- | | | | |
| | 2-yl)-N3-methyl-N3-((4-methylthiazol-2- | | | | |
| | yl)methyl)isophthalamide | | | | |
| 261 | N1-((2S,3R)-4-(5-chloro-2-fluorobenzylamino)-3- | +++ | | | +++ |
| | hydroxy-1-phenylbutan-2-yl)-N3-methyl-N3-((4- | | | | |
| | methylthiazol-2-yl)methyl)isophthalamide | | | | |
| 262 | N1-((2S,3R)-3-hydroxy-4-(3- | +++ | | | +++ |
| | methoxybenzylamino)-1-phenylbutan-2-yl)-5-(1H- | | | | |
| | imidazol-1-yl)-N3-methyl-N3-((4-methylthiazol-2- | | | | |
| | yl)methyl)isophthalamide | | | | |
| 263 | N1-((2S,3R)-3-hydroxy-4-((5-isopropylpyridin-3- | +++ | | | +++ |
| | yl)methylamino)-1-phenylbutan-2-yl)-5-(1H- | | | | |
| | imidazol-2-yl)-N3-methyl-N3-((4-methylthiazol-2- | | | | |
| | yl)methyl)isophthalamide | | | | |
| 264 | N1-((2S,3R)-4-((5-acetylpyridin-3- | +++ | | | +++ |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |
| 265 | N1-((2S,3R)-4-(3-bromo-5- | +++ | | | +++ |
| | (trifluoromethyl)benzylamino)-3-hydroxy-1- | | | | |
| | phenylbutan-2-yl)-N3-methyl-N3-((4- | | | | |
| | methylthiazol-2-yl)methyl)isophthalamide | | | | |
| 266 | N1-((2S,3R)-4-(3-cyano-5- | +++ | | | +++ |
| | (trifluoromethyl)benzylamino)-3-hydroxy-1- | | | | |
| | phenylbutan-2-yl)-N3-methyl-N3-((4- | | | | |
| | methylthiazol-2-yl)methyl)isophthalamide | | | | |
| 267 | N1-((2S,3R)-4-(biphenyl-3-ylmethylamino)-3- | +++ | | | +++ |
| | hydroxy-1-phenylbutan-2-yl)-N3-methyl-N3-((4- | | | | |
| | methylthiazol-2-yl)methyl)isophthalamide | | | | |
| 268 | N1-((2S,3R)-4-(3-tert-butylbenzylamino)-3- | +++ | | | +++ |
| | hydroxy-1-phenylbutan-2-yl)-5-(dimethylamino)- | | | | |
| | N3-methyl-N3-((4-methylthiazol-2- | | | | |
| | yl)methyl)isophthalamide | | | | |
| 269 | 3-(((2R,3S)-2-hydroxy-3-(3-(methyl((4- | +++ | | | +++ |
| | methylthiazol-2-yl)methyl)carbamoyl)benzamido)- | | | | |
| | 4-phenylbutylamino)methyl)-5-(prop-1-en-2- | | | | |
| | yl)pyridine 1-oxide | | | | |
| 270 | N1-((2S,3R)-4-((5-(2-fluoropropan-2-yl)pyridin-3- | +++ | | | +++ |
| | yl)methylamino)-3-hydroxy-1-phenylbutan-2-yl)- | | | | |
| | 5-(furan-3-yl)-N3-methyl-N3-((4-methylthiazol-2- | | | | |
| | yl)methyl)isophthalamide | | | | |
| 271 | N1-((2S,3R)-3-hydroxy-4-((5-isopropylpyridin-3- | +++ | | | +++ |
| | yl)methylamino)-1-phenylbutan-2-yl)-N3-methyl- | | | | |
| | 5-(1-methyl-1H-imidazol-2-yl)-N3-((4- | | | | |
| | methylthiazol-2-yl)methyl)isophthalamide | | | | |
| 272 | 3′-chloro-N3-((2S,3R)-3-hydroxy-4-((5- | +++ | | | +++ |
| | isopropylpyridin-3-yl)methylamino)-1- | | | | |
| | phenylbutan-2-yl)-N5-methyl-N5-((4- | | | | |
| | methylthiazol-2-yl)methyl)biphenyl-3,5- | | | | |
| | dicarboxamide | | | | |
| 273 | N1-((2S,3R)-3-hydroxy-1-phenyl-4-(3- | +++ | | | +++ |
| | (trifluoromethyl)benzylamino)butan-2-yl)-5- | | | | |
| | isopropyl-N3-methyl-N3-((4-methylthiazol-2- | | | | |
| | yl)methyl)isophthalamide | | | | |
| 274 | N1-((2S,3R)-3-hydroxy-4-((5-isopropylpyridin-3- | +++ | | | +++ |
| | yl)methylamino)-1-phenylbutan-2-yl)-5-isopropyl- | | | | |
| | N3-methyl-N3-((4-methylthiazol-2- | | | | |
| | yl)methyl)isophthalamide | | | | |
| 275 | N1-((2S,3R)-3-hydroxy-1-phenyl-4-(3-(pyridin-4- | +++ | | | |
| | yl)-5-(trifluoromethyl)benzylamino)butan-2-yl)- | | | | |
| | N3-methyl-N3-((4-methylthiazol-2- | | | | |
| | yl)methyl)isophthalamide | | | | |
| 276 | N1-((2S,3R)-4-(3-acetamido-5- | +++ | | | +++ |
| | (trifluoromethyl)benzylamino)-3-hydroxy-1- | | | | |
| | phenylbutan-2-yl)-N3-methyl-N3-((4- | | | | |
| | methylthiazol-2-yl)methyl)isophthalamide | | | | |
| 277 | N1-((2S,3R)-4-((5-(2-fluoropropan-2-yl)pyridin-3- | +++ | | | +++ |
| | yl)methylamino)-3-hydroxy-1-phenylbutan-2-yl)- | | | | |
| | 5-isopropyl-N3-methyl-N3-((4-methylthiazol-2- | | | | |
| | yl)methyl)isophthalamide | | | | |
| 278 | N1-((4-(fluoromethyl)thiazol-2-yl)methyl)-N3- | +++ | | | +++ |
| | ((2S,3R)-3-hydroxy-4-((5-isopropylpyridin-3- | | | | |
| | yl)methylamino)-1-phenylbutan-2-yl)-N1- | | | | |
| | methylisophthalamide | | | | |
| 279 | N1-((2S,3R)-3-hydroxy-4-((5-((Z)-1- | ++ | | | +++ |
| | methoxyprop-1-en-2-yl)pyridin-3- | | | | |
| | yl)methylamino)-1-phenylbutan-2-yl)-N3-methyl- | | | | |
| | N3-((4-methylthiazol-2-yl)methyl)isophthalamide | | | | |
| 280 | N1-((4-(fluoromethyl)thiazol-2-yl)methyl)-N3- | +++ | | | +++ |
| | ((2S,3R)-3-hydroxy-1-phenyl-4-((5- | | | | |
| | (trifluoromethyl)pyridin-3-yl)methylamino)butan- | | | | |
| | 2-yl)-N1-methylisophthalamide | | | | |
| 281 | N1-((4-(fluoromethyl)thiazol-2-yl)methyl)-N3- | +++ | | | |
| | ((2S,3R)-4-((5-(2-fluoropropan-2-yl)pyridin-3- | | | | |
| | yl)methylamino)-3-hydroxy-1-phenylbutan-2-yl)- | | | | |
| | N1-methylisophthalamide | | | | |
| 282 | N1-((2S,3R)-3-hydroxy-4-(2-methoxy-5- | + | | | |
| | (trifluoromethyl)benzylamino)-1-phenylbutan-2- | | | | |
| | yl)-N3-methyl-N3-((4-methylthiazol-2- | | | | |
| | yl)methyl)isophthalamide | | | | |
| 283 | N1-((2S,3R)-3-hydroxy-4-(3-(methylamino)-5- | +++ | | | |
| | (trifluoromethyl)benzylamino)-1-phenylbutan-2- | | | | |
| | yl)-N3-methyl-N3-((4-methylthiazol-2- | | | | |
| | yl)methyl)isophthalamide | | | | |
| 284 | N1-((2S,3R)-4-((5-(1,1-dimethoxypropan-2- | +++ | | | |
| | yl)pyridin-3-yl)methylamino)-3-hydroxy-1- | | | | |
| | phenylbutan-2-yl)-N3-methyl-N3-((4- | | | | |
| | methylthiazol-2-yl)methyl)isophthalamide | | | | |
| 285 | N1-((4-(difluoromethyl)thiazol-2-yl)methyl)-N3- | +++ | | | |
| | ((2S,3R)-3-hydroxy-4-((5-isopropylpyridin-3- | | | | |
| | yl)methylamino)-1-phenylbutan-2-yl)-N1- | | | | |
| | methylisophthalamide | | | | |
| 286 | N1-((4-(difluoromethyl)thiazol-2-yl)methyl)-N3- | +++ | | | |
| | ((2S,3R)-4-((5-(2-fluoropropan-2-yl)pyridin-3- | | | | |
| | yl)methylamino)-3-hydroxy-1-phenylbutan-2-yl)- | | | | |
| | N1-methylisophthalamide | | | | |
| 287 | N1-((2S,3R)-3-hydroxy-4-((1-methyl-1H-pyrazol- | +++ | | | |
| | 4-yl)methylamino)-1-phenylbutan-2-yl)-N3- | | | | |
| | methyl-N3-((4-methylthiazol-2- | | | | |
| | yl)methyl)isophthalamide | | | | |
| 288 | N1-((2S,3R)-3-hydroxy-1-phenyl-4-(5-(prop-1-en- | + | | | |
| | 2-yl)nicotinamido)butan-2-yl)-N3-methyl-N3-((4- | | | | |
| | methylthiazol-2-yl)methyl)isophthalamide | | | | |
| 289 | N1-((2S,3R)-3-hydroxy-1-phenyl-4-(3- | +++ | | | |
| | (trifluoromethyl)benzylamino)butan-2-yl)-N3- | | | | |
| | methyl-N3-((4-methylthiazol-2-yl)methyl)-5- | | | | |
| | (oxazol-2-yl)isophthalamide | | | | |
| 290 | N1-((2S,3R)-3-hydroxy-4-((R)-1-(3- | +++ | | | |
| | methoxyphenyl)ethylamino)-1-phenylbutan-2-yl)- | | | | |
| | N3-methyl-N3-((4-methylthiazol-2-yl)methyl)-5- | | | | |
| | (oxazol-2-yl)isophthalamide | | | | |
| 291 | N1-((2S,3R)-3-hydroxy-4-(2-hydroxy-3- | + | | | |
| | (trifluoromethyl)benzylamino)-1-phenylbutan-2- | | | | |
| | yl)-N3-methyl-N3-((4-methylthiazol-2- | | | | |
| | yl)methyl)isophthalamide | | | | |
| 292 | N1-((2S,3R)-3-hydroxy-4-((S)-1-(3- | +++ | | | |
| | methoxyphenyl)ethylamino)-1-phenylbutan-2-yl)- | | | | |
| | N3-methyl-N3-((4-methylthiazol-2-yl)methyl)-5- | | | | |
| | (oxazol-2-yl)isophthalamide | | | | |
| 293 | 5-(difluoromethyl)-N1-((2S,3R)-4-((5-(2- | +++ | | | +++ |
| | fluoropropan-2-yl)pyridin-3-yl)methylamino)-3- | | | | |
| | hydroxy-1-phenylbutan-2-yl)-N3-methyl-N3-((4- | | | | |
| | methylthiazol-2-yl)methyl)isophthalamide | | | | |
| 294 | N1-((2S,3R)-3-hydroxy-4-((4-methylthiazol-2- | + | | | |
| | yl)methylamino)-1-phenylbutan-2-yl)-N3-methyl- | | | | |
| | N3-((4-methylthiazol-2-yl)methyl)isophthalamide | | | | |
| 295 | N1-((2S,3R)-3-hydroxy-4-((5-isopropylpyridin-3- | +++ | | | |
| | yl)methylamino)-1-phenylbutan-2-yl)-N3-methyl- | | | | |
| | N3-((4-methylthiazol-2-yl)methyl)-5-(1H-pyrazol- | | | | |
| | 4-yl)isophthalamide | | | | |
| 296 | 2-hydroxy-N1-((2S,3R)-3-hydroxy-4-(3- | + | | | |
| | methoxybenzylamino)-1-phenylbutan-2-yl)-N3- | | | | |
| | methyl-N3-((4-methylthiazol-2- | | | | |
| | yl)methyl)isophthalamide | | | | |
| 297 | 2-hydroxy-N1-((2S,3R)-3-hydroxy-4-((5- | + | | | |
| | isopropylpyridin-3-yl)methylamino)-1- | | | | |
| | phenylbutan-2-yl)-N3-methyl-N3-((4- | | | | |
| | methylthiazol-2-yl)methyl)isophthalamide | | | | |
| 298 | 4′-(dimethylamino)-N3-((2S,3R)-3-hydroxy-4-((5- | +++ | | | |
| | isopropylpyridin-3-yl)methylamino)-1- | | | | |
| | phenylbutan-2-yl)-N5-methyl-N5-((4- | | | | |
| | methylthiazol-2-yl)methyl)biphenyl-3,5- | | | | |
| | dicarboxamide | | | | |
| 299 | 5-(3,3-dimethoxypyrrolidin-1-yl)-N1-((2S,3R)-3- | +++ | | | +++ |
| | hydroxy-4-((5-isopropylpyridin-3- | | | | |
| | yl)methylamino)-1-phenylbutan-2-yl)-N3-methyl- | | | | |
| | N3-((4-methylthiazol-2-yl)methyl)isophthalamide | | | | |
| 300 | N1-((2S,3R)-3-hydroxy-4-((5-isopropylpyridin-3- | +++ | | | |
| | yl)methylamino)-1-phenylbutan-2-yl)-5-(1H- | | | | |
| | imidazol-1-yl)-N3-methyl-N3-((4-methylthiazol-2- | | | | |
| | yl)methyl)isophthalamide | | | | |
| 301 | 5-(difluoromethyl)-N1-((2S,3R)-3-hydroxy-4-((S)- | +++ | | | |
| | 1-(3-methoxyphenyl)ethylamino)-1-phenylbutan- | | | | |
| | 2-yl)-N3-methyl-N3-((4-methylthiazol-2- | | | | |
| | yl)methyl)isophthalamide | | | | |
| 302 | N1-((2S,3R)-3-hydroxy-1-phenyl-4-((4- | +++ | | | |
| | (trifluoromethyl)pyridin-2-yl)methylamino)butan- | | | | |
| | 2-yl)-N3-methyl-N3-((4-methylthiazol-2- | | | | |
| | yl)methyl)isophthalamide | | | | |
| 303 | 5-(furan-3-yl)-N1-((2S,3R)-3-hydroxy-4-((5- | +++ | | | |
| | isopropylpyridin-3-yl)methylamino)-1- | | | | |
| | phenylbutan-2-yl)-N3-methyl-N3-((4- | | | | |
| | methylthiazol-2-yl)methyl)isophthalamide | | | | |
| 304 | N1-((2S,3R)-3-hydroxy-4-((5- | +++ | | | |
| | (isopropylamino)pyridin-3-yl)methylamino)-1- | | | | |
| | phenylbutan-2-yl)-N3-methyl-N3-((4- | | | | |
| | methylthiazol-2-yl)methyl)isophthalamide | | | | |
| 305 | 5-(furan-2-yl)-N1-((2S,3R)-3-hydroxy-1-phenyl-4- | +++ | | | |
| | (3-(trifluoromethyl)benzylamino)butan-2-yl)-N3- | | | | |
| | methyl-N3-((4-methylthiazol-2- | | | | |
| | yl)methyl)isophthalamide | | | | |
| 306 | 5-acetyl-N1-((2S,3R)-1-(3,5-difluorophenyl)-3- | +++ | | | |
| | hydroxy-4-(3-methoxybenzylamino)butan-2-yl)- | | | | |
| | N3-((R)-1-phenylethyl)isophthalamide | | | | |
| 307 | N1-((2S,3R)-3-hydroxy-4-(3- | + | | | |
| | methoxybenzylamino)-1-phenylbutan-2-yl)-5-(3- | | | | |
| | hydroxycyclopentyl)-N3-methyl-N3-((4- | | | | |
| | methylthiazol-2-yl)methyl)isophthalamide | | | | |
| 308 | 5-acetyl-N1-((2S,3R)-3-hydroxy-4-((5- | +++ | | | |
| | isopropylpyridin-3-yl)methylamino)-1- | | | | |
| | phenylbutan-2-yl)-N3-((R)-1- | | | | |
| | phenylethyl)isophthalamide | | | | |
| 309 | N1-((2S,3R)-3-hydroxy-4-(3- | + | | | |
| | methoxyphenylsulfonamido)-1-phenylbutan-2-yl)- | | | | |
| | N3-methyl-N3-((4-methylthiazol-2- | | | | |
| | yl)methyl)isophthalamide | | | | |
| 310 | N1-((2S,3R)-3-hydroxy-1-phenyl-4-(3- | + | | | |
| | (trifluoromethyl)phenylsulfonamido)butan-2-yl)- | | | | |
| | N3-methyl-N3-((4-methylthiazol-2- | | | | |
| | yl)methyl)isophthalamide | | | | |
| 311 | 3-(((2R,3S)-2-hydroxy-3-(3-(methyl((4- | +++ | | | |
| | methylthiazol-2-yl)methyl)carbamoyl)benzamido)- | | | | |
| | 4-phenylbutylamino)methyl)-5-isopropylphenyl 2- | | | | |
| | (dimethylamino)acetate | | | | |
| 312 | N1-((2S,3R)-3-hydroxy-4-(((5-isopropylpyridin-3- | + | | | |
| | yl)methyl)(methyl)amino)-1-phenylbutan-2-yl)- | | | | |
| | N3-methyl-N3-((4-methylthiazol-2- | | | | |
| | yl)methyl)isophthalamide | | | | |
| 313 | N1-((2S,3R)-3-hydroxy-4-((5-(1-hydroxypropan- | +++ | | | |
| | 2-yl)pyridin-3-yl)methylamino)-1-phenylbutan-2- | | | | |
| | yl)-N3-methyl-N3-((4-methylthiazol-2- | | | | |
| | yl)methyl)isophthalamide | | | | |
| 314 | N1-((2S,3R)-3-hydroxy-4-((5-(1-hydroxypropan- | +++ | | | |
| | 2-yl)pyridin-3-yl)methylamino)-1-phenylbutan-2- | | | | |
| | yl)-N3-methyl-N3-((4-methylthiazol-2- | | | | |
| | yl)methyl)isophthalamide | | | | |
| 315 | N1-((4-bromothiazol-2-yl)methyl)-N3-((2S,3R)-3- | +++ | | | |
| | hydroxy-4-((5-isopropylpyridin-3- | | | | |
| | yl)methylamino)-1-phenylbutan-2-yl)-N1- | | | | |
| | methylisophthalamide | | | | |
| 316 | N1-((2S,3R)-3-hydroxy-4-((5-isopropylpyridin-3- | +++ | | | |
| | yl)methylamino)-1-phenylbutan-2-yl)-N3-((4- | | | | |
| | (hydroxymethyl)thiazol-2-yl)methyl)-N3- | | | | |
| | methylisophthalamide | | | | |
| 317 | tert-butyl 3′-((2S,3R)-3-hydroxy-1-phenyl-4-(3- | +++ | | | |
| | (trifluoromethyl)benzylamino)butan-2- | | | | |
| | ylcarbamoyl)-5′-(methyl((4-methylthiazol-2- | | | | |
| | yl)methyl)carbamoyl)biphenyl-3- | | | | |
| | yl(methyl)carbamate | | | | |
| 318 | N1-((2S,3R)-3-hydroxy-4-((5-isopropylpyridin-3- | +++ | | | |
| | yl)methylamino)-1-phenylbutan-2-yl)-N3-methyl- | | | | |
| | N3-(thiazol-2-ylmethyl)isophthalamide | | | | |
| 319 | N1-((2S,3R)-3-hydroxy-1-phenyl-4-((5-(prop-1- | +++ | | | |
| | en-2-yl)pyridin-3-yl)methylamino)butan-2-yl)-N3- | | | | |
| | ((4-(hydroxymethyl)thiazol-2-yl)methyl)-N3- | | | | |
| | methylisophthalamide | | | | |
| 320 | N1-((2S,3R)-4-((1-ethyl-1H-pyrazol-4- | +++ | | | |
| | yl)methylamino)-3-hydroxy-1-phenylbutan-2-yl)- | | | | |
| | N3-methyl-N3-((4-methylthiazol-2- | | | | |
| | yl)methyl)isophthalamide | | | | |

In Table 1, for the Ki data, a “+” represents a Ki of greater than >501 nM, a “++” represents a Ki from 500 nm to 301 nm, and a “+++” represents a Ki of less than 300 nm. For the IC50 data, a “+” represents an IC50 of greater than 5 μM, a “++” represents an IC50 from 1 to 5 μM, and a “+++” represents an IC50 of less than 1 μM. For example, N1-((2S,3R)-3-hydroxy-4-((5-isopropylpyridin-3-yl)methylamino)-1-phenylbutan-2-yl)-N3,5-dimethyl-N3-((4-methylthiazol-2-yl)methyl)isophthalamide has a M2 Ki=16.8 nM, CathD Ki=32.3 nM, M1 Ki=264.39 nM, and IC50 =0.006 μM, corresponding to +++ for all values depicted in Table 1.

### Example 3

### Physical Characterization Data for Inhibitors

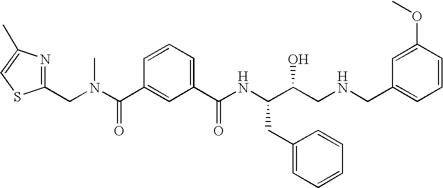

N¹-((2S,3R)-3-hydroxy-4-((3-methoxybenzyl)(methyl)amino)-1-phenylbutan-2-yl)-N³-methyl-N³-((4-methylthiazol-2-yl)methyl)isophthalamide: ¹H NMR (300 MHz) 7.66-7.72 (m, 3H), 7.43 (m, 1H), 7.19-7.29 (m, 6H), 6.81-6.90 (m, 4H), 4.99 (m, 2H), 4.42 (m, 1H), 3.82-3.87 (m, 1H), 3.81 (s, 3H), 3.41-3.62 (m, 2H), 2.98-3.00 (m, 5H), 2.66-2.70 (m, 2H), 2.47 (s, 3H), 2.27 (s, 3H).

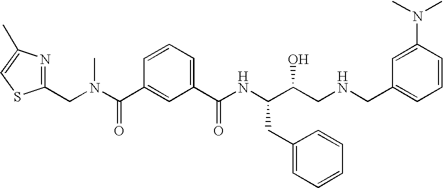

N1-((2S,3R)-4-(3-(dimethylamino)benzylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.86-7.79 (m, 1H), 7.68-7.35 (m, 3H), 7.31-7.19 (m, 7H), 6.90 (broad s, 1H), 6.70-6.65 (m, 3H), 4.98 (broad s, 1.3H), 4.66 (broad s, 0.6H), 4.47-4.38 (m, 1H), 3.85-3.72 (m, 3H), 3.17-2.86 (m, 15H), 2.47 (s, 3H).

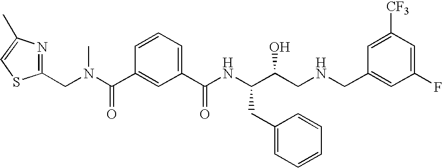

N1-((2S,3R)-4-(3-fluoro-5-(trifluoromethyl)benzylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.721-7.503 (m, 3H), 7.407-7.115 (m, 9H), 6.874 (s, 1H), 4.937 (br, 1.4H), 4.627 (br, 0.6H), 4.331 (m, 1H), 3.827 (m, 2H), 3.698 (m, 1H), 3.077-2.894 (m, 5H), 2.738 (m, 2H), 2.427 (s, 3H).

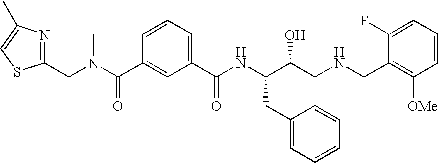

N1-((2S,3R)-4-(2-fluoro-6-methoxybenzylamino)-3-hydroxy-1-phenylbutan-2-yl)-N3-methyl-N3-((4-methylthiazol-2-yl)methyl)isophthalamide: ¹H NMR (300 MHz) 7.63 (m, 1H), 7.37 (m, 1H), 7.16-7.29 (m, 7H), 6.97 (m, 1H), 6.85-6.89 (m, 2H), 6.73-6.78 (m, 1H), 4.97 (m, 2H), 4.42 (m, 1H), 3.83 (s, 2H), 3.76 (s, 3H), 3.74 (m, 1H), 2.97-3.12 (m, 5H), 2.83 (s, 2H), 2.46 (s, 3H).

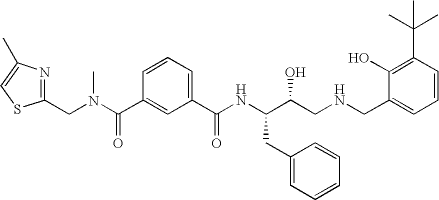

N1-((2S,3R)-4-(3-tert-butyl-2-hydroxybenzylamino)-3-hydroxy-1-phenylbutan-2-yl)-N3-methyl-N3-((4-methylthiazol-2-yl)methyl)isophthalamide: ¹H NMR (300 MHz) 7.64 (m, 1H), 7.43 (m, 1H), 7.12-7.30 (m, 8H), 6.89-6.92 (m, 2H), 6.78 (m, 1H), 4.99 (m, 2H), 4.34 (m, 1H), 3.95-4.11 (m, 3H), 2.89-3.14 (m, 5H), 2.77-2.85 (m, 2H), 2.48 (s, 3H), 1.45 (s, 9H).

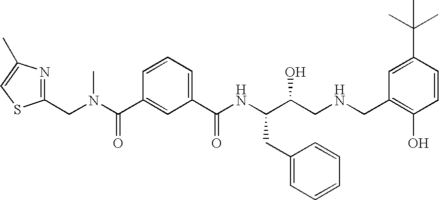

N1-((2S,3R)-4-(5-tert-butyl-2-hydroxybenzylamino)-3-hydroxy-1-phenylbutan-2-yl)-N3-methyl-N3-((4-methylthiazol-2-yl)methyl)isophthalamide: ¹H NMR (300 MHz) 7.68 (s, 1H), 7.61-7.63 (m, 1H), 7.32-7.37 (m, 1H), 7.17-7.25 (m, 7H), 7.01 (m, 1H), 6.89 (s, 1H), 6.76 (m, 1H), 4.94 (m, 2H), 4.32 (m, 1H), 3.88-4.06 (m, 3H), 2.95-3.10 (m, 2H), 3.97 (s, 3H), 2.78-2.81 (m, 2H), 2.47 (s, 3H), 1.29 (s, 9H).

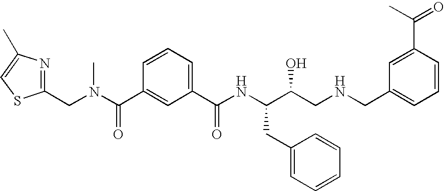

N1-((2S,3R)-4-(3-acetylbenzylamino)-3-hydroxy-1-phenylbutan-2-yl)-N3-methyl-N3-((4-methylthiazol-2-yl)methyl)isophthalamide: ¹H NMR (300 MHz) 7.93 (m, 1H), 7.85 (m, 1H), 7.63 (m, 1H), 7.56 (m, 1H), 7.38-7.46 (m, 2H), 7.19-7.29 (m, 7H), 6.90 (s, 1H), 4.97 (m, 2H), 4.41 (m, 1H), 3.82-3.92 (m, 2H), 3.70 (m, 1H), 3.00-3.02 (m, 5H), 2.80 (m, 2H), 2.61 (s, 3H), 2.46 (s, 3H).

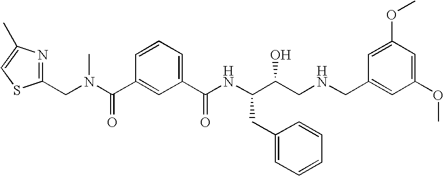

N1-((2S,3R)-4-(3,5-dimethoxybenzylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.713-7.148 (m, 9H), 6.887 (s, 1H), 6.462 (m 2H), 6.350 (m, 1H), 4.939 (br, 1.5H), 4.639 (br, 0.5H), 3.784-3.652 (m, 9H), 3.094-2.877 (m, 4H), 2.784 (s, 2 H), 2.430 (s, 3H).

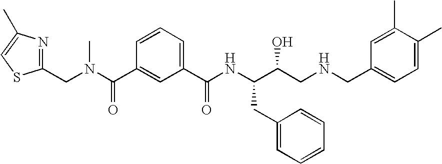

N1-((2S,3R)-4-(3,4-dimethylbenzylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.754-7.549 (m, 4H), 7.426-7.027 (m, 8H), 6.908 (s, 1H), 4.966 (br, 1.3H), 4.663 (br, 0.7H), 4.368 (m, 1H), 3.721 (m, 3H), 3.111-2.876 (m, 5H), 2.821 (m, 2 H), 2.458 (s, 3H), 2.236 (m, 6H).

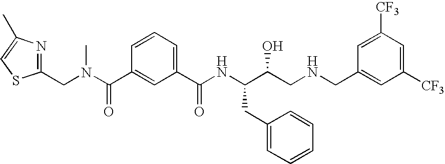

N1-((2S,3R)-4-(3,5-bis(trifluoromethyl)benzylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.797-7.508 (m, 5H), 7.426-7.115 (m, 7H), 6.881 (s, 1H), 4.929 (br, 1.4H), 4.631 (br, 0.6H), 4.315 (m, 1H), 3.905 (m, 2H), 3.738 (m, 1H), 3.090-2.826 (m, 5 H), 2.760 (m, 2H), 2.424 (s, 3H).

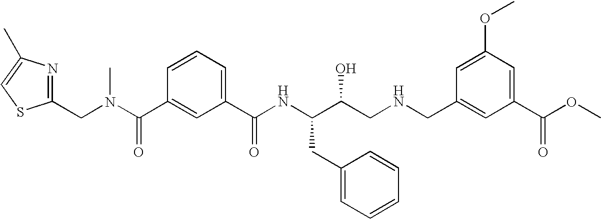

methyl 3-(((2R,3S)-2-hydroxy-3-(3-(methyl((4-methylthiazol-2-yl)methyl)carbamoyl)benzamido)-4-phenylbutylamino)methyl)-5-methoxybenzoate: ¹H NMR (300 MHz, CDCl₃+CD₃OD), d: 7.829-7.301 (m, 6H), 7.298-7.162 (m, 5H), 7.114 (m, 1H), 6.894 (s, 1H), 4.968 (br, 1.3H), 4.650 (br, 0.7H), 4.408 (m, 1H), 3.916 (s, 3H), 3.878-3.765 (m, 2H), 3.846 (s, 3H), 3.716 (m, 1H), 3.124-3.003 (m, 5H), 2.810 (m, 2H), 2.462 (s, 3H).

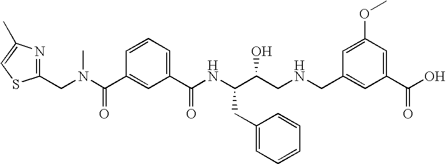

3-(((2R,3S)-2-hydroxy-3-(3-(methyl((4-methylthiazol-2-yl)methyl)carbamoyl)benzamido)-4-phenylbutylamino)methyl)-5-methoxybenzoic acid: ¹H NMR (300 MHz, CDCl₃+CD₃OD), d: 7.924-7.353 (m, 6H), 7.233-6.903 (m, 7H), 4.929 (br, 1.4H), 4.627 (br, 0.6H), 4.334 (m, 1H), 4.197 (m, 1H), 4.022 (m, 2H), 3.722 (s, 3H), 3.343-2.832 (m, 7H), 2.433 (s, 3H).

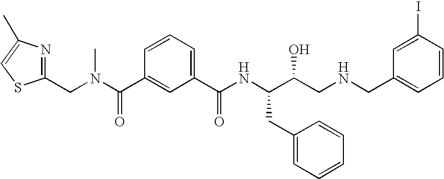

N1-((2S,3R)-3-hydroxy-4-(3-iodobenzylamino)-1-phenylbutan-2-yl)-N3-methyl-N3-((4-methylthiazol-2-yl)methyl)isophthalamide: ¹H NMR (300 MHz, CDCl₃+CD₃OD), d: 7.732-6.912 (m, 14H), 4.975 (br, 1.4H), 4.671 (br, 0.6H), 4.369 (m, 1H), 3.744 (m, 3H), 3.125-2.905 (m, 5H), 2.789 (m, 2H), 2.457 (s, 3H).

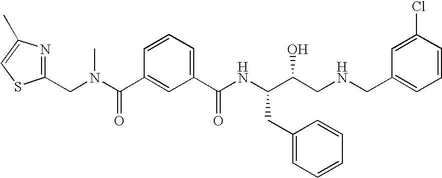

N1-((2S,3R)-4-(3-chlorobenzylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.721-7.147 (m, 13H), 6.901 (s, 1H), 4.954 (br, 1.4H), 4.56 (br, 0.6H), 4.360 (m, 1H), 3.743 (m, 3H), 3.113-2.891 (m, 5H), 2.778 (d, J=4.5, 2H), 2.447 (s, 3H).

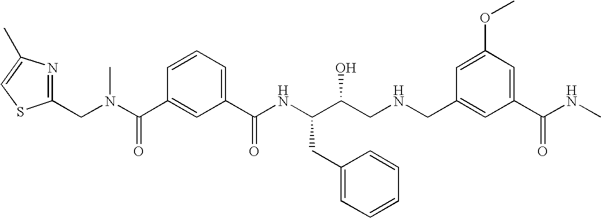

N1-((2S,3R)-3-hydroxy-4-(3-methoxy-5-(methylcarbamoyl)benzylamino)-1-phenylbutan-2-yl)-N3-methyl-N3-((4-methylthiazol-2-yl)methyl)isophthalamide: ¹H NMR (300 MHz, CDCl₃+CD₃OD), d: 7.675-7.389 (m, 3H), 7.330-7.167 (m, 9H), 6.970 (s, 1H), 4.964 (br, 1.4H), 4.646 (br, 0.6H), 4.310 (m, 1H), 3.909-3.752 (m, 6H), 3.111-2.792 (m, 10H), 2.452 (s, 3H).

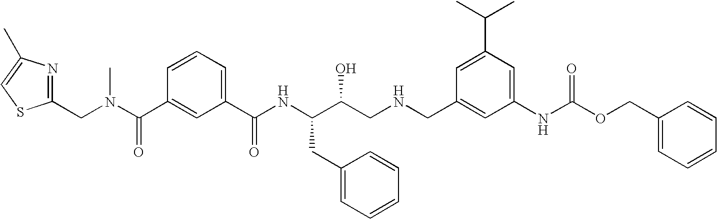

benzyl 3-(((2R,3S)-2-hydroxy-3-(3-(methyl((4-methylthiazol-2-yl)methyl)carbamoyl)benzamido)-4-phenylbutylamino)methyl)-5-isopropylphenylcarbamate: ¹H NMR (300 MHz, CDCl₃) d 7.72-7.86 (m, 1H), 7.67-7.69 (m, 1H), 7.45-7.63 (m, 1H), 7.07-7.45 (m, 16H), 6.82-6.92 (m, 3H), 5.19 (s, 2H), 4.96 (s, 2H), 4.38-4.50 (m, 1H), 3.67-3.85 (m, 3H), 2.78-3.15 (m, 8H), 2.47 (s, 3H), 1.21-1.24 (m, 6H).

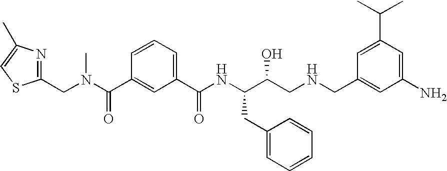

N1-((2S,3R)-4-(3-amino-5-isopropylbenzylamino)-3-hydroxy-1-phenylbutan-2-yl)-N3-methyl-N3-((4-methylthiazol-2-yl)methyl)isophthalamide: ¹H NMR (300 MHz, CDCl₃) d 7.68-7.84 (m, 1H), 7.61-7.64 (m, 1H), 7.43-7.59 (m, 1H), 7.31-7.37 (m, 1H), 7.06-7.29 (m, 8H), 6.77-6.87 (m, 2H), 6.43-6.54 (m, 2H), 6.26-6.43 (m, 1H), 4.90 (s, 2H), 4.27-4.40 (m, 1H), 3.62-3.81 (m, 3H), 2.84-3.12 (m, 5H), 2.59-2.84 (m, 3H), 2.38 (s, 3H), 1.07-1.10 (m, 6H).

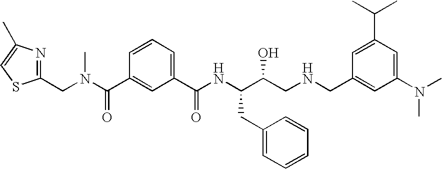

N1-((2S,3R)-4-(3-(dimethylamino)-5-isopropylbenzylamino)-3-hydroxy-1-phenylbutan-2-yl)-N3-methyl-N3-((4-methylthiazol-2-yl)methyl)isophthalamide: ¹H NMR (300 MHz, CDCl₃) d 7.78-7.90 (m, 1H), 7.69-7.72 (m, 1H), 7.55-7.66 (m, 1H), 7.39-7.45 (m, 1H), 7.19-7.36 (m, 7H), 6.89-6.94 (m, 1H), 6.59-6.60 (m, 2H), 6.53-6.58 (m, 1H), 5.01 (s, 2H), 4.37-4.52 (m, 1H), 3.74-3.93 (m, 3H), 2.96 (s, 6H), 2.80-3.21 (m, 8H), 2.48 (s, 3H), 1.24-1.26 (m, 6H).

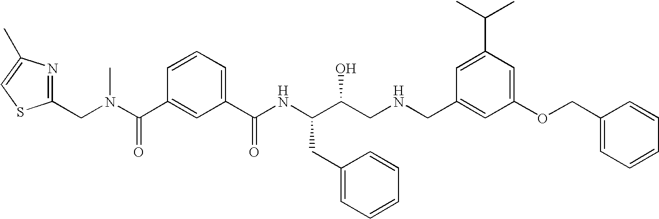

N1-((2S,3R)-4-(3-(benzyloxy)-5-isopropylbenzylamino)-3-hydroxy-1-phenylbutan-2-yl)-N3-methyl-N3-((4-methylthiazol-2-yl)methyl)isophthalamide: ¹H NMR (300 MHz, CDCl₃) d 7.74-7.87 (m, 1H), 7.66-7.69 (m, 1H), 7.52-7.61 (m, 1H), 7.30-7.48 (m, 6H), 7.15-7.30 (m, 8H), 6.85-6.93 (m, 1H), 6.79-6.85 (m, 3H), 5.06 (s, 2H), 4.99 (s, 2H), 4.37-4.49 (m, 1H), 3.66-3.74 (m, 2H), 3.74-3.87 (m, 1H), 2.94-3.18 (m, 5H), 2.80-2.93 (m, 3H), 2.48 (s, 3H), 1.23-1.26 (m, 6H).

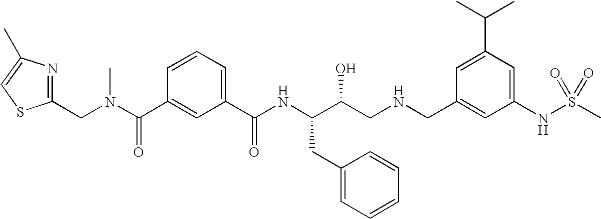

N1-((2S,3R)-3-hydroxy-4-(3-isopropyl-5-(methylsulfonamido)benzylamino)-1-phenylbutan-2-yl)-N3-methyl-N3-((4-methylthiazol-2-yl)methyl)isophthalamide: ¹H NMR (300 MHz, CDCl₃) d 7.63-7.83 (m, 3H), 7.55-7.63 (m, 1H), 7.41-7.48 (m, 1H), 7.20-7.36 (m, 6H), 7.02-7.09 (m, 2H), 6.88-6.97 (m, 2H), 5.03 (s, 2H), 4.42-4.52 (m, 1H), 3.67-3.86 (m, 3H), 3.13-3.23 (m, 1H), 2.92, (s, 3H), 2.96-3.13 (m, 5H), 2.80-2.92 (m, 3H), 2.48 (s, 3H), 1.22-1.24 (m, 6H).

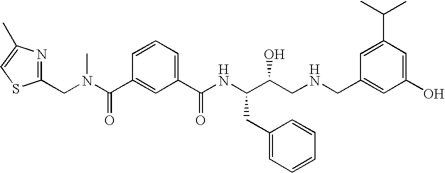

N1-((2S,3R)-3-hydroxy-4-(3-hydroxy-5-isopropylbenzylamino)-1-phenylbutan-2-yl)-N3-methyl-N3-((4-methylthiazol-2-yl)methyl)isophthalamide: ¹H NMR (300 MHz, CDCl₃) d 7.77-7.87 (m, 1H), 7.61-7.77 (m, 3H), 7.52-7.54 (m, 1H), 7.35-7.41 (m, 1H), 7.18-7.34 (m, 6H), 6.89-6.96 (m, 1H), 6.66-6.76 (m, 1H), 6.62 (s, 3H), 4.94-5.11 (m, 2H), 4.42-4.55 (m, 1H), 3.64-3.87 (m, 4H), 3.14-3.25 (m, 1H), 2.99-3.12 (m, 2H), 2.86-2.99 (m, 2H), 2.73-2.86 (m, 1H), 2.49 (s, 3H), 1.18-1.20 (m, 6H).

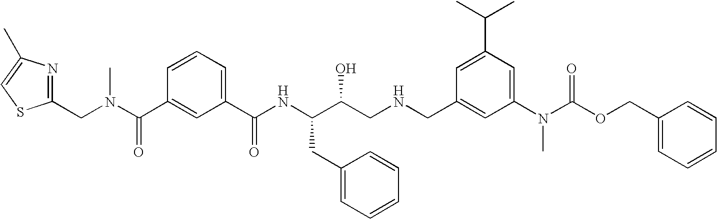

benzyl 3-(((2R,3S)-2-hydroxy-3-(3-(methyl((4-methylthiazol-2-yl)methyl)carbamoyl)benzamido)-4-phenylbutylamino)methyl)-5-isopropylphenyl(methyl)carbamate: ¹H NMR (300 MHz, CDCl₃) d 7.73-7.86 (m, 1H), 7.66-7.69 (m, 1H), 7.52-7.63 (m, 1H), 7.39-7.44 (m, 1H), 7.16-7.37 (m, 12H), 6.96-7.11 (m, 3H), 6.86-6.96 (m, 1H), 5.17 (s, 2H), 4.99 (s, 2H), 4.34-4.47 (m, 1H), 3.75-3.87 (m, 2H), 3.65-3.74 (m, 1H), 3.33 (s, 3H), 2.95-3.20 (m, 5H), 2.77-2.95 (m, 3H), 2.47 (s, 3H), 1.22-1.24 (m, 6H).

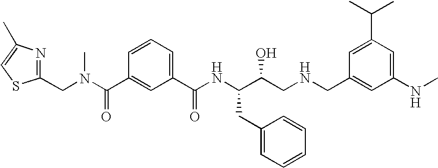

N1-((2S,3R)-3-hydroxy-4-(3-isopropyl-5-(methylamino)benzylamino)-1-phenylbutan-2-yl)-N3-methyl-N3-((4-methylthiazol-2-yl)methyl)isophthalamide: ¹H NMR (300 MHz, CDCl₃) d 7.75-7.90 (m, 1H), 7.70-7.73 (m, 1H), 7.54-7.69 (m, 1H), 7.40-7.45 (m, 1H), 7.13-7.38 (m, 8H), 6.87-6.95 (m, 1H), 6.56 (s, 1H), 6.49 (s, 1H), 6.42 (s, 1H), 5.00 (s, 2H), 4.36-4.49 (m, 1H), 3.62-3.91 (m, 3H), 2.83 (s, 3H), 2.75-3.22 (m, 8H), 2.48 (s, 3H), 1.21-1.24 (m, 6H).

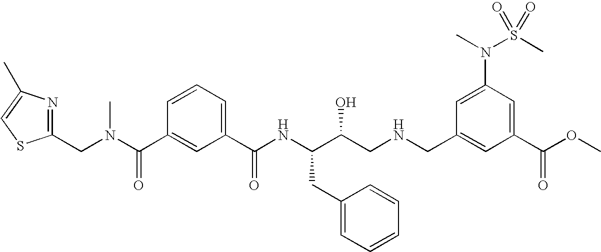

methyl 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoate: ¹H NMR (300 MHz, CDCl₃) d 7.96 (s, 1H), 7.90-7.91 (m, 1H), 7.71-7.83 (m, 1H), 7.65-7.71 (m, 2H), 7.54-7.65 (m, 1H), 7.42-7.47 (m, 1H), 7.17-7.32 (m, 6H), 6.92 (s, 1H), 6.67-6.89 (m, 1H), 5.00 (s, 2H), 4.34-4.46 (m, 1H), 3.95 (s, 3H), 3.90 (s, 2H), 3.64-3.75 (m, 1H), 3.36 (s, 3H), 2.95-3.19 (m, 5H), 2.85 (s, 3H), 2.79-2.81 (m, 2H), 2.48 (s, 3H).

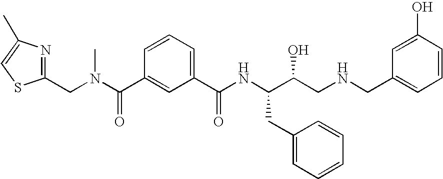

N1-((2S,3R)-3-hydroxy-4-(3-hydroxybenzylamino)-1-phenylbutan-2-yl)-N3-methyl-N3-((4-methylthiazol-2-yl)methyl)isophthalamide: ¹H NMR (300 MHz, CDCl₃): 2.47 (s, 3H), 2.84-3.00 (m, 7H), 3.64-3.82 (m, 3H), 4.38-4.48 (m, 1H), 4.67 (br s, 1H), 4.94-5.01 (m, 1H), 6.72-6.74 (m, 1H), 6.87-6.92 (m, 2H), 7.08-7.34 (m, 8H), 7.46-7.49 (m, 1H), 7.59-7.62 (m, 1H), 7.77-7.80 (m, 1H).

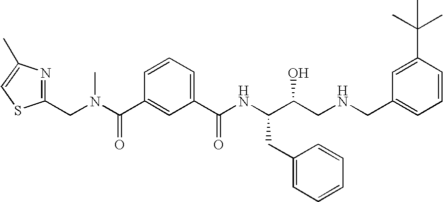

N1-((2S,3R)-4-(3-tert-butylbenzylamino)-3-hydroxy-1-phenylbutan-2-yl)-N3-methyl-N3-((4-methylthiazol-2-yl)methyl)isophthalamide: ¹H NMR (300 MHz, CDCl₃): 1.32 (s, 9H), 2.48 (s, 3H), 2.85-315 (m, 7H), 3.77-3.94 (m, 3H), 4.39-4.48 (m, 1H), 4.68 (br s, 1H), 4.99 (br s, 1H), 6.91 (s, 1H), 7.17-7.44 (m, 10H), 7.66-7.69 (m, 2H), 7.78 (s, 1H).

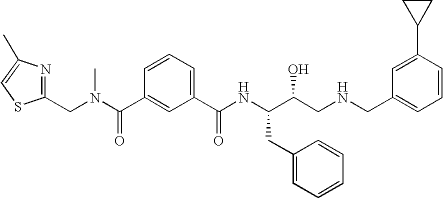

N1-((2S,3R)-4-(3-cyclopropylbenzylamino)-3-hydroxy-1-phenylbutan-2-yl)-N3-methyl-N3-((4-methylthiazol-2-yl)methyl)isophthalamide: ¹H NMR (300 MHz, CDCl₃): 0.62-0.75 (m, 1H), 0.81-1.06 (m, 2H), 1.68-1.82 (m, 2H), 2.43 (s, 3H), 2.72-3.16 (m, 7H), 3.65-3.79 (m, 3H), 4.36-4.42 (m, 1H), 4.62 (s, 1H), 5.01 (s, 1H), 6.82-7.79 (m, 14H).

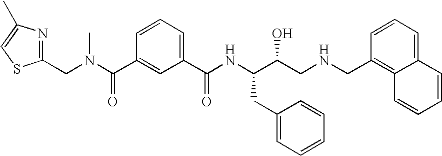

|1-((2S,3R)-3-hydroxy-4-(naphthalen-1-ylmethylamino)-1-phenylbutan-2-yl)-N3-methyl-N3-((4-methylthiazol-2-yl)methyl)isophthalamide: ¹H NMR (300 MHz, CDCl₃+CD₃OD), d: 7.713-7.503 (m, 3H), 7.407-7.112 (m, 9H), 6.874 (s, 1H), 4.937 (br, 1.3H), 4.627 (br, 0.7H), 4.344 (m, 1H), 3.827 (m, 2H), 3.698 (m, 1H), 3.077-2.894 (m, 5 H), 2.738 (m, 2H), 2.427 (s, 3H).

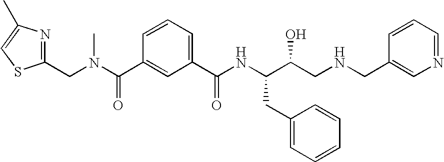

N1-((2S,3R)-3-hydroxy-1-phenyl-4-(pyridin-3-ylmethylamino)butan-2-yl)-N3-methyl-N3-((4-methylthiazol-2-yl)methyl)isophthalamide: ¹H NMR (300 MHz, CDCl₃) d 8.60 (s, 1H), 8.53-8.55 (m, 1H), 7.71-7.89 (m, 1H), 7.66-7.69 (m, 1H), 7.54-7.63 (m, 1H), 7.41-7.46 (m, 1H), 7.17-7.36 (m, 8H), 6.83-6.99 (m, 2H), 5.00 (s, 2H), 4.34-4.49 (m, 1H), 3.84-3.94 (m, 2H), 3.70-3.82 (m, 1H), 2.95-3.22 (m, 5H), 2.78-2.93 (m, 2H), 2.48 (s, 3H).

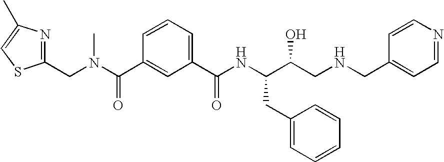

N1-((2S,3R)-3-hydroxy-1-phenyl-4-(pyridin-4-ylmethylamino)butan-2-yl)-N3-methyl-N3-((4-methylthiazol-2-yl)methyl)isophthalamide: ¹H NMR (300 MHz, CDCl₃) d 8.57-8.59 (m, 2H), 7.72-7.89 (m, 1H), 7.67-7.69 (m, 1H), 7.55-7.64 (m, 1H), 7.43-7.48 (m, 1H), 7.19-7.37 (m, 8H), 6.85-6.96 (m, 1H), 6.62-6.85 (m, 1H), 5.01 (s, 2H), 4.35-4.48 (m, 1H), 3.84-3.97 (m, 2H), 3.66-3.78 (m, 1H), 2.96-3.26 (m, 5H), 2.75-2.96 (m, 2H), 2.49 (s, 3H).

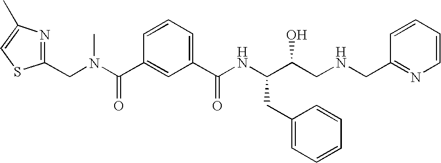

N1-((2S,3R)-3-hydroxy-1-phenyl-4-(pyridin-2-ylmethylamino)butan-2-yl)-N3-methyl-N3-((4-methylthiazol-2-yl)methyl)isophthalamide: ¹H NMR (300 MHz, CDCl₃) d 8.55-8.57 (m, 1H), 7.79-7.93 (m, 1H), 7.65-7.79 (m, 2H), 7.53-7.65 (m, 2H), 7.41-7.46 (m, 1H), 7.04-7.35 (m, 7H), 6.88-6.96 (m, 2H), 5.00 (s, 2H), 4.41-4.58 (m, 1H), 4.01-4.17 (m, 2H), 3.80-3.95 (m, 1H), 2.92-3.22 (m, 7H), 2.48 (s, 3H).

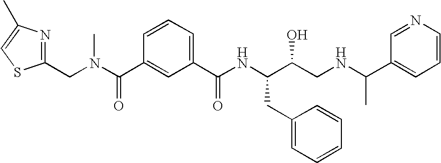

N1-((2S,3R)-3-hydroxy-1-phenyl-4-(1-(pyridin-3-yl)ethylamino)butan-2-yl)-N3-methyl-N3-((4-methylthiazol-2-yl)methyl)isophthalamide: ¹H NMR (300 MHz, CDCl₃+CD₃OD), d: 8.462 (m, 2H), 7.704-7.142 (m, 11H), 6.875 (s, 1H), 4.943 (br, 1.3H), 4.636 (br, 0.7H), 4.329 (m, 1H), 3.777 (m, 1H), 3.619 (m, 1H), 3.092-2.871 (m, 5H), 2.721-2.495 (m, 2H), 2.429 (s, 3H), 1.417 (m, 3H).

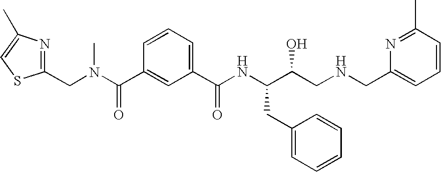

N1-((2S,3R)-3-hydroxy-4-((6-methylpyridin-2-yl)methylamino)-1-phenylbutan-2-yl)-N3-methyl-N3-((4-methylthiazol-2-yl)methyl)isophthalamide: ¹H NMR (300 MHz, CDCl₃) δ 7.77-7.53 (m, 3H), 7.43-7.38 (m, 1H), 7.30-7.05 (m, 9H), 6.92 (broad s, 1H), 5.00 (broad s, 1.3H), 4.67 (broad s, 0.5H), 4.54-4.55 (m, 1H), 4.02-3.91 (m, 2H), 3.74-3.70 (m, 1H), 3.13-2.91 (m, 7H) 2.55 (s, 3H), 2.48 (s, 3H).

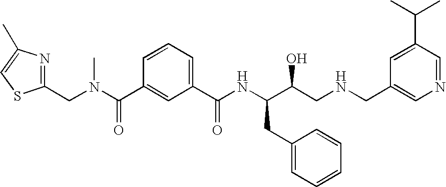

N1-((2R,3S)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz) 8.36-8.38 (m, 2H), 7.75 (s, 1H), 7.64 (m, 1H), 7.52 (m, 2H), 7.39 (m, 1H), 7.19-7.29 (m, 5H), 6.89 (s, 1H), 4.97 (m, 2H), 4.41 (m, 1H), 3.72-3.81 (m, 3H), 2.97-3.12 (m, 5H), 2.92 (m, 1H), 2.80-2.82 (m, 2H), 2.46 (s, 3H), 1.25 (m, 6H).

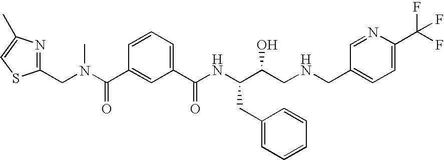

N1-((2S,3R)-3-hydroxy-1-phenyl-4-((6-(trifluoromethyl)pyridin-3-yl)methylamino)butan-2-yl)-N3-methyl-N3-((4-methylthiazol-2-yl)methyl)isophthalamide: ¹H NMR (300 MHz, CDCl₃+CD₃OD), d: 8.649 (s, 1H), 7.880-7.164 (m, 11H), 6.891 (s, 1 H), 4.947 (br, 1.3H), 4.639 (br, 0.7H), 4.336 (m, 1H), 3.924-3.827 (m, 2H), 3.723 (m, 1H), 2.989 (s 3H), 3.092-2.890 (m, 2H), 2.753 (s, 2H), 2.437 (s, 3H).

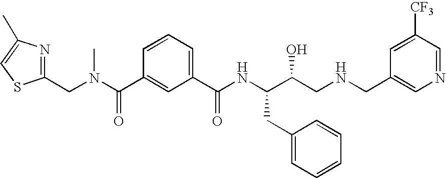

N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide: ¹H NMR (300 MHz, CDCl₃+CD₃OD), d: 8.707 (m, 2H), 7.935 (s, 1H), 7.659-7.114 (m, 9 H), 6.878 (s, 1H), 4.922 (br, 1.3H), 4.624 (br, 0.7H), 4.306 (m, 1H), 3.859 (s, 2H), 3.719 (m, 1H), 3.080-2.864 (m, 5H), 2.736 (m, 2H), 2.417 (s, 3H).

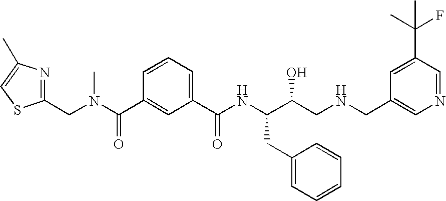

N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 8.502 (m, 2H), 7.749-7.155 (m, 10H), 6.916 (s, 1H), 4.973 (br, 1.4H), 4.674 (br, 0.6H), 4.379 (m, 1H), 3.908-3.742 (m, 3H), 3.129-2.918 (m, 5H), 2.812 (m, 2H), 2.461 (s, 3H), 1.738 (s, 3H), 1.665 (s, 3H).

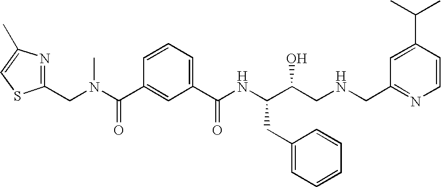

N1-((2S,3R)-3-hydroxy-4-((4-isopropylpyridin-2-yl)methylamino)-1-phenylbutan-2-yl)-N3-methyl-N3-((4-methylthiazol-2-yl)methyl)isophthalamide: ¹H NMR (300 MHz, CDCl₃) d 8.44-8.46 (m, 1H), 7.77-7.92 (m, 1H), 7.72-7.75 (m, 1H), 7.55-7.66 (m, 1H), 7.41-7.46 (m, 1H), 7.18-7.35 (m, 7H), 7.13 (s, 1H), 7.08-7.10 (m, 2H), 6.88-6.95 (m, 1H), 5.00 (s, 2H), 4.44-4.55 (m, 1H), 3.98-4.09 (m, 2H), 3.78-3.87 (m, 1H), 2.84-3.20 (m, 8H), 2.48 (s, 3H), 1.26-1.28 (m, 6H).

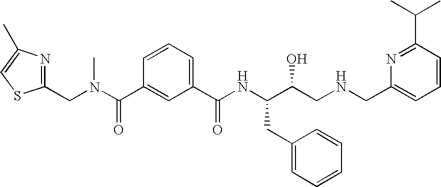

N1-((2S,3R)-3-hydroxy-4-((6-isopropylpyridin-2-yl)methylamino)-1-phenylbutan-2-yl)-N3-methyl-N3-((4-methylthiazol-2-yl)methyl)isophthalamide: ¹H NMR (300 MHz, CDCl₃) d 7.75-8.00 (m, 1H), 7.56-7.75 (m, 2H), 7.42-7.56 (m, 2H), 7.07-7.42 (m, 10H), 6.89-7.02 (m, 1H), 4.94-5.15 (m, 2H), 4.46-4.64 (m, 1H), 4.01-4.26 (m, 2H), 3.85-4.01 (m, 1H), 2.92-3.32 (m, 8H), 2.40-2.58 (m, 3H), 1.18-1.49 (m, 6H).

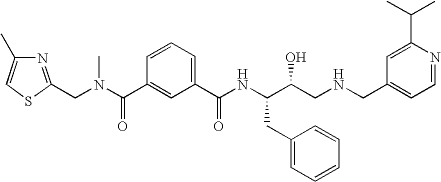

N1-((2S,3R)-3-hydroxy-4-((2-isopropylpyridin-4-yl)methylamino)-1-phenylbutan-2-yl)-N3-methyl-N3-((4-methylthiazol-2-yl)methyl)isophthalamide: ¹H NMR (300 MHz, CDCl₃) d 8.49-8.50 (m, 1H), 7.72-7.86 (m, 1H), 7.66-7.69 (m, 1H), 7.53-7.64 (m, 1H), 7.41-7.46 (m, 1H), 7.19-7.38 (m, 6H), 7.15 (s, 1H), 7.10-7.11 (m, 1H), 6.72-6.96 (m, 2H), 4.99 (s, 2H), 4.35-4.50 (m, 1H), 3.79-3.90 (m, 2H), 3.65-3.76 (m, 1H), 2.97-3.20 (m, 6H), 2.75-2.88 (m, 2H), 2.48 (s, 3H), 1.29-1.32 (m, 6H).

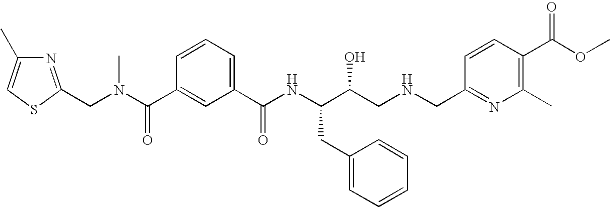

methyl 6-(((2R,3S)-2-hydroxy-3-(3-(methyl((4-methylthiazol-2-yl)methyl)carbamoyl)benzamido)-4-phenylbutylamino)methyl)-2-methylnicotinate: ¹H NMR (300 MHz, CDCl₃) d 8.19-8.21 (m, 1H), 7.75-7.87 (m, 1H), 7.70-7.72 (m, 1H), 7.54-7.67 (m, 1H), 7.41-7.46 (m, 1H), 7.16-7.36 (m, 6H), 6.85-7.06 (m, 2H), 5.00 (s, 2H), 4.42-4.56 (m, 1H), 4.00-4.10 (m, 2H), 3.95 (s, 3H), 3.66-3.83 (m, 1H), 2.84 (s, 3H), 2.75-3.22 (m, 8H), 2.48 (s, 3H).

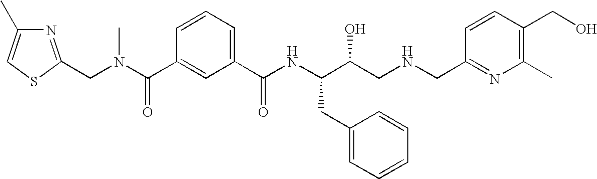

N1-((2S,3R)-3-hydroxy-4-((5-(hydroxymethyl)-6-methylpyridin-2-yl)methylamino)-1-phenylbutan-2-yl)-N3-methyl-N3-((4-methylthiazol-2-yl)methyl)isophthalamide: ¹H NMR (300 MHz, CDCl₃) d 7.15-7.95 (m, 11H), 6.95-7.15 (m, 1H), 6.84-6.95 (m, 2H), 4.85-5.05 (m, 2H), 4.43-4.75 (m, 5H), 3.95-4.16 (m, 2H), 3.80-3.95 (m, 1H), 2.84-3.32 (m, 7H), 2.44-2.60 (m, 3H), 2.35 (s, 3H).

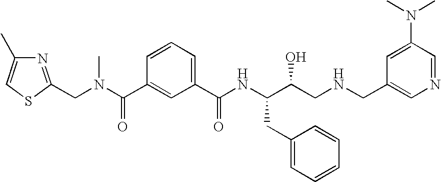

N1-((2S,3R)-4-((5-(dimethylamino)pyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃) d 8.03 (s, 1H), 7.92 (s, 1H), 7.74-7.89 (m, 1H), 7.66-7.69 (m, 1H), 7.51-7.64 (m, 1H), 7.38-7.43 (m, 1H), 7.14-7.32 (m, 7H), 6.99-7.04 (m, 1H), 6.85-6.94 (m, 1H), 4.98 (s, 2H), 4.33-4.47 (m, 1H), 3.71-3.90 (m, 3H), 2.97 (s, 6H), 2.76-3.16 (m, 7H), 2.47 (s, 3H).

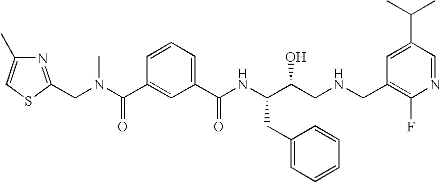

N1-((2S,3R)-4-((2-fluoro-5-isopropylpyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃) d 8.00 (s, 1H), 7.56-7.86 (m, 4H), 7.43-7.48 (m, 1H), 7.18-7.36 (m, 6H), 6.88-6.97 (m, 1H), 6.69-6.88 (m, 1H), 5.01 (s, 2H), 4.34-4.50 (m, 1H), 3.85-3.96 (m, 2H), 3.67-3.82 (m, 1H), 2.91-3.22 (m, 6H), 2.81-2.91 (m, 2H), 2.48 (s, 3H), 1.27-1.29 (m, 6H).

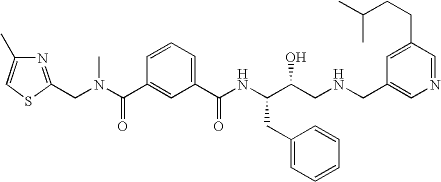

N1-((2S,3R)-3-hydroxy-4-((5-isopentylpyridin-3-yl)methylamino)-1-phenylbutan-2-yl)-N3-methyl-N3-((4-methylthiazol-2-yl)methyl)isophthalamide: ¹H NMR (300 MHz, CDCl₃) d 8.37-8.40 (m, 2H), 7.72-7.86 (m, 1H), 7.67-7.69 (m, 1H), 7.55-7.65 (m, 1H), 7.51 (s, 1H), 7.42-7.47 (m, 1H), 7.18-7.34 (m, 7H), 6.87-6.99 (m, 2H), 5.00 (s, 2H), 4.36-4.47 (m, 1H), 3.79-3.88 (m, 2H), 3.67-3.78 (m, 1H), 2.96-3.20 (m, 4H), 2.82-2.83 (m, 2H), 2.59-2.65 (m, 2H), 2.48 (s, 3H), 1.56-1.68 (m, 1H), 1.46-1.56 (m, 2H), 0.95-0.97 (m, 6H).

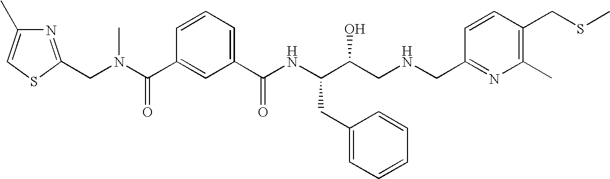

N1-((2S,3R)-3-hydroxy-4-((6-methyl-5-(methylthiomethyl)pyridin-2-yl)methylamino)-1-phenylbutan-2-yl)-N3-methyl-N3-((4-methylthiazol-2-yl)methyl)isophthalamide: ¹H NMR (300 MHz, CDCl₃) d 7.79-7.89 (m, 1H), 7.71-7.79 (m, 1H), 7.55-7.64 (m, 1H), 7.48-7.51 (m, 1H), 7.40-7.45 (m, 1H), 7.15-7.35 (m, 8H), 7.08-7.11 (m, 1H), 6.88-6.95 (m, 1H), 5.00 (s, 2H), 4.45-4.56 (m, 1H), 3.95-4.20 (m, 2H), 3.80-3.95 (m, 1H), 3.67 (s, 2H), 2.94-3.21 (m, 7H), 2.59 (s, 3H), 2.48 (s, 3H), 2.05 (s, 3H).

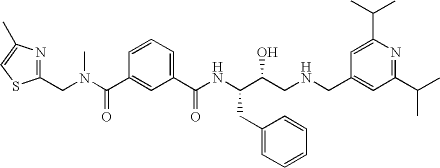

N1-((2S,3R)-4-((2,6-diisopropylpyridin-4-yl)methylamino)-3-hydroxy-1-phenylbutan-2-yl)-N3-methyl-N3-((4-methylthiazol-2-yl)methyl)isophthalamide: ¹H NMR (300 MHz, CDCl₃) d 7.73-7.86 (m, 1H), 7.67-7.70 (m, 1H), 7.59-7.65 (m, 1H), 7.43-7.48 (m, 1H), 7.20-7.35 (m, 7H), 6.96 (s, 2H), 6.86-6.94 (m, 2H), 5.01 (s, 2H), 4.40-4.48 (m, 1H), 3.77-3.88 (m, 2H), 3.67-3.77 (m, 1H), 2.98-3.22 (m, 7H), 2.79-2.86 (m, 2H), 2.49 (s, 3H), 1.28-1.31 (m, 12H).

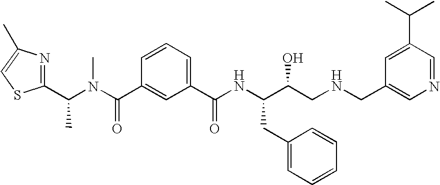

N1-((2S,3R)-3-hydroxy-4-((5-isopropylpyridin-3-yl)methylamino)-1-phenylbutan-2-yl)-N3-methyl-N3-((R)-1-(4-methylthiazol-2-yl)ethyl)isophthalamide: ¹H NMR (300 MHz, CDCl₃): 1.27 (d, 6H, J=6.9 Hz), 1.61-1.82 (m, 3H), 2.47 (s, 3H), 2.81-3.05 (m, 8H), 3.66-3.87 (m, 4H), 4.36-4.48 (m, 1H), 5.00-5.02 (m, 1H), 6.90 (s, 1H), 7.21-7.30 (m, 6H), 7.42-7.77 (m, 5H), 8.39-8.41 (m, 1H).

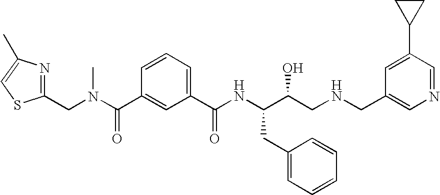

N1-((2S,3R)-4-((5-cyclopropylpyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃): 0.62-0.76 (m, 1H), 0.94-1.06 (m, 2H), 1.76-1.92 (m, 2H), 2.47 (s, 3H), 2.78 (br s, 2H), 2.82-3.22 (m, 5H), 3.62-3.81 (m, 3H), 4.28-4.42 (m, 1H), 4.60 (br s, 1H), 4.94 (br s, 1H), 6.81 (s, 1H), 7.12-7.49 (m, 7H), 7.50-7.80 (m, 3H), 8.3 (d, 2H, J=6.0 Hz).

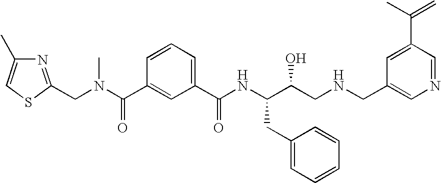

N1-((2S,3R)-3-hydroxy-1-phenyl-4-((5-(prop-1-en-2-yl)pyridin-3-yl)methylamino)butan-2-yl)-N3-methyl-N3-((4-methylthiazol-2-yl)methyl)isophthalamide: ¹H NMR (300 MHz, CDCl₃): 2.20 (s, 3H), 2.37 (s, 3H), 2.78-3.18 (m, 7H), 3.58-3.84 (m, 3H), 4.80 (s, 1H), 5.01 (s, 1H), 5.18 (s, 1H), 5.38 (s, 1H), 6.90 (s, 1H), 7.14-7.76 (m, 10H), 8.41 (s, 1H), 8.61 (s, 1H).

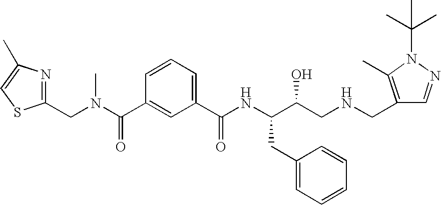

N1-((2S,3R)-4-((1-tert-butyl-5-methyl-1H-pyrazol-4-yl)methylamino)-3-hydroxy-1-phenylbutan-2-yl)-N3-methyl-N3-((4-methylthiazol-2-yl)methyl)isophthalamide: ¹H NMR (300 MHz) 7.78 (m, 1H), 7.72 (m, 1H), 7.70 (m, 1H), 7.41-7.47 (m, 2H), 7.24-7.35 (m, 5H), 6.93 (s, 1H), 5.00 (m, 2H), 4.40 (m, 1H), 3.65-3.77 (m, 3H), 3.03-3.15 (m, 5H), 2.78-2.94 (m, 2H), 2.48 (s, 3H), 2.44 (s, 3H), 1.64 (s, 9H).

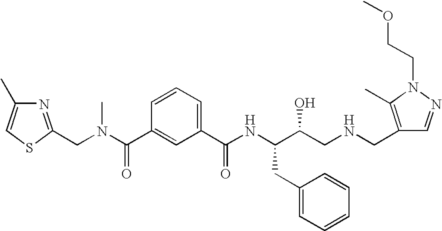

N1-((2S,3R)-3-hydroxy-4-((1-(2-methoxyethyl)-5-methyl-1H-pyrazol-4-yl)methylamino)-1-phenylbutan-2-yl)-N3-methyl-N3-((4-methylthiazol-2-yl)methyl)isophthalamide: ¹H NMR (300 MHz) 7.81 (m, 1H), 7.73-7.75 (m, 2H), 7.57 (m, 1H), 7.39-7.44 (m, 1H), 7.18-7.29 (m, 5H), 6.92 (s, 1H), 4.99 (m, 2H), 4.36 (m, 1H), 4.16-4.19 (m, 2H), 3.82-3.95 (m, 2H), 3.70-3.73 (m, 3H), 3.30 (s, 3H), 3.01-3.14 (m, 5H), 2.48 (s, 3H), 2.22 (s, 3H).

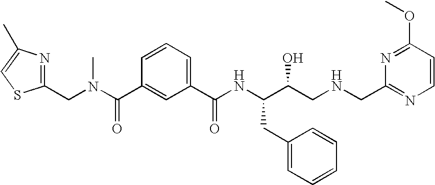

N1-((2S,3R)-3-hydroxy-4-((4-methoxypyrimidin-2-yl)methylamino)-1-phenylbutan-2-yl)-N3-methyl-N3-((4-methylthiazol-2-yl)methyl)isophthalamide: ¹H NMR (300 MHz, CDCl₃): 2.48 (s, 3H), 2.94-3.14 (m, 7H), 3.77-3.79 (m, 1H), 3.95-4.05 (m, 5H), 4.48-4.54 (m, 1H), 4.67 (br s, 1H), 4.99 (br s, 1H), 6.63 (d, 1H, J=6.0 Hz), 6.92 (s, 1H), 7.21-7.30 (m, 6H), 7.40-7.45 (m, 1H), 7.70-7.77 (m, 2H), 8.37 (d, 1H, J=6.0 Hz).

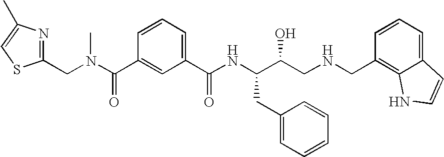

N1-((2S,3R)-4-((1H-indol-7-yl)methylamino)-3-hydroxy-1-phenylbutan-2-yl)-N3-methyl-N3-((4-methylthiazol-2-yl)methyl)isophthalamide: ¹H NMR (300 MHz, CDCl₃): 2.38 (s, 3H), 2.80-3.06 (m, 7H), 3.64-3.76 (m, 1H), 4.02-4.09 (m, 2H), 4.28-4.34 (m, 1H), 4.53 (brs, 1H), 4.90 (br s, 1H), 6.45 (s, 1H), 6.95-7.66 (m, 14H).

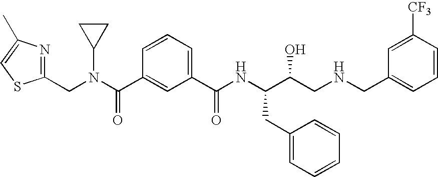

N1-cyclopropyl-N3-((2S,3R)-3-hydroxy-1-phenyl-4-(3-(trifluoromethyl)benzylamino)butan-2-yl)-N1-((4-methylthiazol-2-yl)methyl)isophthalamide: ¹H NMR (300 MHz, CDCl₃) δ 7.79-7.17 (m, 15H), 6.85-6.77 (m, 2H), 5.00 (broad s, 2H), 4.43-4.36 (m, 1H), 3.94-3.67 (m, 3H), 3.03-2.78 (m, 6H), 2.45 (s, 3H), 0.57-0.484 (m, 4H).

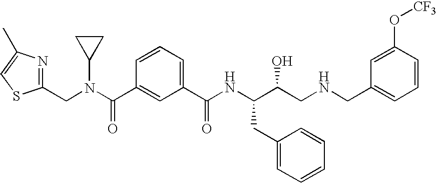

N1-cyclopropyl-N3-((2S,3R)-3-hydroxy-1-phenyl-4-(3-(trifluoromethoxy)benzylamino)butan-2-yl)-N1-((4-methylthiazol-2-yl)methyl)isophthalamide: ¹H NMR (300 MHz, CDCl₃+CD₃OD), d: 7.834 (s, 1H), 7.693-7.626 (m, 2H), 7.402-7.105 (m, 10H), 6.863 (m, 1H), 5.011 (br, 2H), 4.418 (m, 1H), 3.888-3.702 (m, 3H), 3.011 (m, 2H), 2.870-2.753 (m, 3H), 2.466 (s, 3H), 0.589-0.469 (m, 4H).

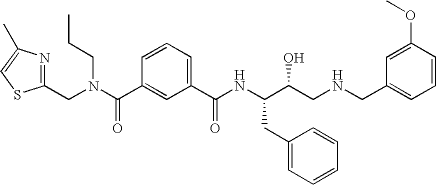

N1-((2S,3R)-3-hydroxy-4-(3-methoxybenzylamino)-1-phenylbutan-2-yl)-N3-((4-methylthiazol-2-yl)methyl)-N3-propylisophthalamide: ¹H NMR (300 MHz, CDCl₃) δ 7.71-7.25 (m, 12H), 6.99-6.83 (m, 5H), 5.00 (broad s, 1.6H), 4.68 (broad s, 0.7H), 4.43 (m, 1H), 3.88-3.69 (m, 5H), 3.54-3.23 (m, 1H), 3.04-2.83 (m, 3H), 2.47 (broad s, 2H), 1.58 (m, 1H), 0.98-0.74 (m, 3H).

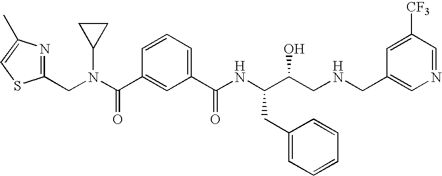

N1-cyclopropyl-N3-((2S,3R)-3-hydroxy-1-phenyl-4-((5-(trifluoromethyl)pyridin-3-yl)methylamino)butan-2-yl)-N1-((4-methylthiazol-2-yl)methyl)isophthalamide: ¹H NMR (300 MHz, CDCl₃+CD₃OD), d: 8.762 (m, 2H), 7.979 (s, 1H), 7.779-7.636 (m, 3H), 7.417 (m, 2H), 7.237-7.139 (m, 4H), 6.883 (m, 1H), 4.988 (m, 2H), 4.354 (m, 1H), 3.901 (s, 2 H), 3.785 (m, 1H), 3.120-2.755 (m, 5H), 2.461 (s, 3H), 0.590-0.461 (m, 4H).

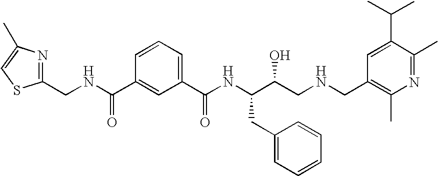

N1-((2S,3R)-3-hydroxy-4-((5-isopropyl-2,6-dimethylpyridin-3-yl)methylamino)-1-phenylbutan-2-yl)-N3-((4-methylthiazol-2-yl)methyl)isophthalamide: ¹H NMR (300 MHz, CDCl₃) d 7.72-7.87 (m, 1H), 7.65-7.67 (m, 1H), 7.55-7.63 (m, 1H), 7.40-7.48 (m, 3H), 7.19-7.35 (m, 6H), 6.87-6.95 (m, 1H), 6.65-6.87 (m, 1H), 5.00 (s, 2H), 4.36-4.48 (m, 1H), 3.80-3.90 (m, 2H), 3.68-3.80 (m, 1H), 2.98-3.19 (m, 6H), 2.80-2.90 (m, 2H), 2.56 (s, 6H), 2.48 (s, 3H), 1.24-1.21 (m, 6H).

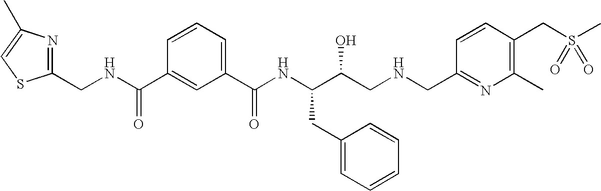

N1-((2S,3R)-3-hydroxy-4-((6-methyl-5-(methylsulfonylmethyl)pyridin-2-yl)methylamino)-1-phenylbutan-2-yl)-N3-((4-methylthiazol-2-yl)methyl)isophthalamide: ¹H NMR (300 MHz, CDCl₃) d 7.77-7.87 (m 1H), 7.71-7.74 (m, 1H), 7.66-7.69 (m, 1H), 7.54-7.62 (m, 1H), 7.41-7.46 (m, 1H), 7.15-7.34 (m, 8H), 6.89-6.97 (m, 1H), 4.99 (s, 2H), 4.41-4.55 (m, 1H), 4.33 (s, 2H), 3.99-4.08 (m, 2H), 3.77-3.91 (m, 1H), 2.91 (s, 3H), 2.90-3.21 (m, 7H), 2.66 (s, 3H), 2.49 (s, 3H).

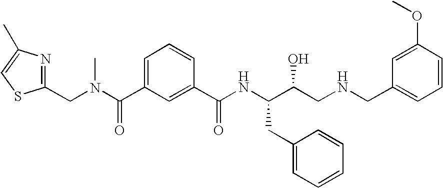

N1-((4,5-dimethylthiazol-2-yl)methyl)-N3-((2S,3R)-3-hydroxy-4-(3-methoxybenzylamino)-1-phenylbutan-2-yl)-N1-methylisophthalamide: ¹H NMR (300 MHz, CDCl₃+CD₃OD): 2.28 (s, 3H), 2.31 (s, 3H), 2.62-3.20 (m, 7H), 3.60-3.86 (m, 3H), 3.76 (s, 3H), 4.22-4.40 (m, 1H), 4.80-4.98 (m, 2H), 6.72-6.90 (m, 4H), 7.05-7.20 (m, 6H), 7.20-7.90 (m, 4H).

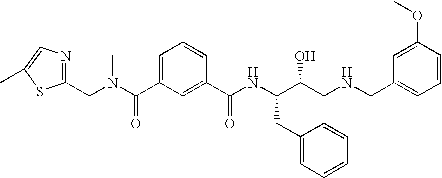

N1-((2S,3R)-3-hydroxy-4-(3-methoxybenzylamino)-1-phenylbutan-2-yl)-N3-methyl-N3-((5-methylthiazol-2-yl)methyl)isophthalamide: ¹H NMR (300 MHz, CDCl₃+CD₃OD): 2.44 (s, 3H), 2.62-3.20 (m, 7H), 3.60-3.86 (m, 3H), 3.76 (s, 3H), 4.22-4.40 (m, 1H), 4.80-4.98 (m, 2H), 6.72-6.90 (m, 4H), 7.05-7.20 (m, 6H), 7.20-7.90 (m, 4H).

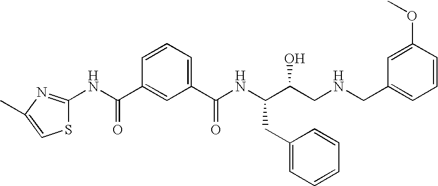

N1-((2S,3R)-3-hydroxy-4-(3-methoxybenzylamino)-1-phenylbutan-2-yl)-N3-(4-methylthiazol-2-yl)isophthalamide: ¹H NMR (300 MHz, CDCl₃) δ 8.40 (s, 1H), 8.07-8.05 (m, 1H), 7.83-7.74 (m, 2H), 7.42-7.37 (m, 1H), 7.23-7.17 (m, 6H), 6.85-6.78 (m, 3H), 6.56 (s, 1H), 4.53-4.49 (m, 1H), 3.89-3.70 (m, 7H), 3.01-2.86 (m, 4H), 2.28 (s, 3H).

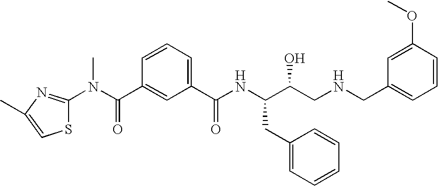

N1-((2S,3R)-3-hydroxy-4-(3-methoxybenzylamino)-1-phenylbutan-2-yl)-N3-methyl-N3-(4-methylthiazol-2-yl)isophthalamide: ¹H NMR (300 MHz, CDCl₃) δ 8.62-8.61 (m, 1H), 8.48-8.55 (m, 1H), 7.83-7.79 (m, 1H), 7.49-7.44 (m, 1H), 7.31-7.22 (m, 7H), 7.00-6.79 (m, 4H), 6.38 (m, 1H), 4.50-4.45 (m, 1H), 3.90-3.71 (m, 9H), 3.09-3.07 (m, 2H), 2.87-2.86 (m, 2H), 2.38 (m, 3H).

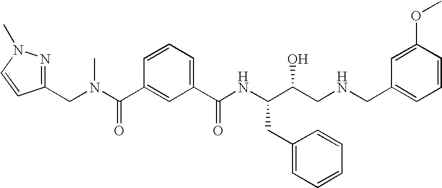

N1-((2S,3R)-3-hydroxy-4-(3-methoxybenzylamino)-1-phenylbutan-2-yl)-N3-methyl-N3-((1-methyl-1H-pyrazol-3-yl)methyl)isophthalamide: ¹H NMR (300 MHz, CDCl₃) δ 7.87-7.20 (m, 15H), 6.95-6.83 (m, 4H), 6.33 (broad s, 0.5H), 6.12 (broad s, 0.5H), 4.76 (broad s, 1.1H), 4.41 (m, 1.9H), 3.92-3.70 (m, 9H), 3.10-2.85 (m, 7H).

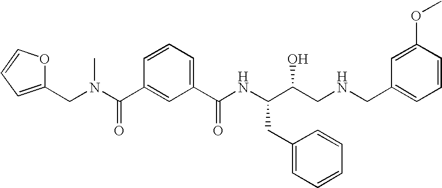

N1-(furan-2-ylmethyl)-N3-((2S,3R)-3-hydroxy-4-(3-methoxybenzylamino)-1-phenylbutan-2-yl)-N1-methylisophthalamide: ¹H NMR (300 MHz, CDCl₃+CD₃OD): 2.40-3.20 (m, 7H), 3.60-3.86 (m, 3H), 3.78 (s, 3H), 4.20-4.45 (m, 2H), 4.60-4.80 (m, 1H), 6.14-6.20 (m, 2H), 6.64-6.92 (m, 4H), 7.05-7.24 (m, 5H), 7.24-7.80 (m, 5H).

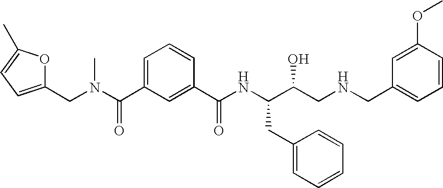

N1-((2S,3R)-3-hydroxy-4-(3-methoxybenzylamino)-1-phenylbutan-2-yl)-N3-methyl-N3-((5-methylfuran-2-yl)methyl)isophthalamide: ¹H NMR (300 MHz, CDCl₃+CD₃OD): 2.10-2.30 (m, 3H), 2.62-3.20 (m, 7H), 3.60-3.86 (m, 3H), 3.78 (s, 3H), 4.20-4.45 (m, 2H), 4.50-4.70 (m, 1H), 5.80-6.20 (m, 2H), 6.70-6.88 (m, 4H), 7.05-7.24 (m, 5H), 7.24-7.80 (m, 5H).

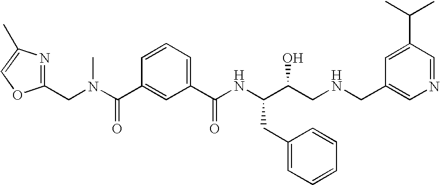

N1-((2S,3R)-3-hydroxy-4-((5-isopropylpyridin-3-yl)methylamino)-1-phenylbutan-2-yl)-N3-methyl-N3-((4-methyloxazol-2-yl)methyl)isophthalamide: ¹H NMR (300 MHz, CDCl₃+CD₃OD): 1.24 (d, 6H, J=6.9 Hz), 2.17 (s, 3H), 2.78 (d, 3H, J=4.8 Hz), 2.84-3.16 (m, 5H), 3.74-82 (m, 3H), 4.24-4.40 (m, 1H), 4.78-4.82 (m, 2H) 7.10-7.30 (m, 5H), 7.40-7.80 (m, 6H), 8.34-8.38 (m, 2H).

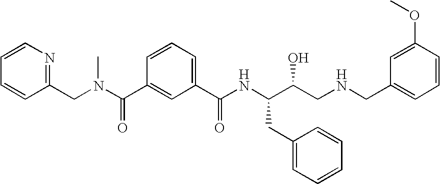

N1-((2S,3R)-3-hydroxy-4-(3-methoxybenzylamino)-1-phenylbutan-2-yl)-N3-methyl-N3-(pyridin-2-ylmethyl)isophthalamide: ¹H NMR (300 MHz, CDCl₃) δ 8.55 (s, 1H), 7.88-7.00 (m, 15H), 6.90-6.78 (m, 3H), 4.85 (broad s, 0.9H), 4.52 (broad s, 0.9H), 4.39-4.37 (m, 1H), 3.82-3.72 (m, 6H), 3.05-2.76 (m, 9H).

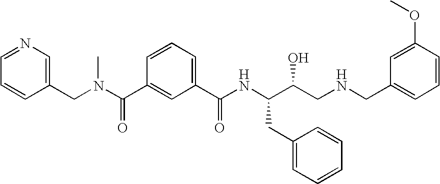

N1-((2S,3R)-3-hydroxy-4-(3-methoxybenzylamino)-1-phenylbutan-2-yl)-N3-methyl-N3-(pyridin-3-ylmethyl)isophthalamide: ¹H NMR (300 MHz, CDCl₃) δ 8.63-8.58 (m, 2H), 7.77-7.18 (m, 13H), 6.93-6.81 (m, 3H), 4.76 (broad s, 1.3H), 4.46-4.41 (m, 1.6H), 3.83-3.73 (m, 6H), 3.02-3.00 (m, 3H), 2.88-2.85 (m, 4H).

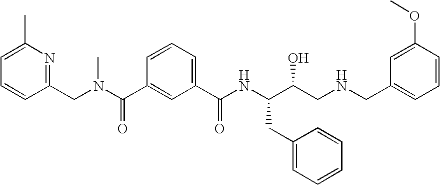

N1-((2S,3R)-3-hydroxy-4-(3-methoxybenzylamino)-1-phenylbutan-2-yl)-N3-methyl-N3-((6-methylpyridin-2-yl)methyl)isophthalamide: ¹H NMR (300 MHz, CDCl₃) δ 7.83-7.78 (m, 1H), 7.65-7.59 (m, 3H), 7.44-7.19 (m, 8H), 7.12-7.10 (m, 1H), 6.98-6.81 (m, 4H), 4.87 (broad s, 1.0H), 4.53 (broad s, 1.0H), 4.44-4.42 (m, 1H), 3.87-3.68 (m, 6H), 3.11-2.98 (m, 5H), 2.85-2.80 (m, 4H), 2.59-2.54 (m, 3H).

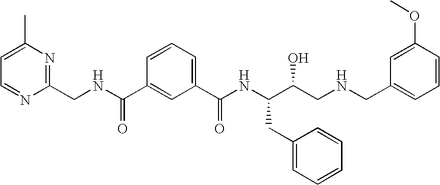

N1-((2S,3R)-3-hydroxy-4-(3-methoxybenzylamino)-1-phenylbutan-2-yl)-N3-((4-methylpyrimidin-2-yl)methyl)isophthalamide: ¹H NMR (300 MHz, CDCl₃): 2.48 (s, 3H), 2.74-2.95 (m, 4H), 3.66-3.79 (m, 6H), 4.34-4.45 (m, 1H), 4.75-4.76 (d, 2H, J=4.8 Hz), 6.71-6.75 (m, 1H), 6.81-6.84 (m, 1H), 7.01-7.02 (d, 1H, J=5.1 Hz), 7.11-7.21 (m, 6H), 7.33 (t, 1H, J=7.8 Hz), 7.65-7.67 (m, 1H), 7.74-7.78 (m, 1H), 7.89-7.91 (m, 1H), 8.14 (br s, 1H), 8.46-8.48 (d, 1H, 7.8 Hz).

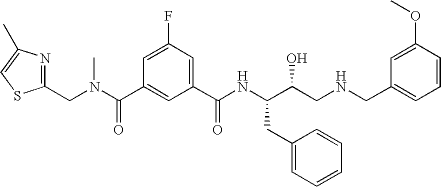

5-fluoro-N1-((2S,3R)-3-hydroxy-4-(3-methoxybenzylamino)-1-phenylbutan-2-yl)-N3-methyl-N3-((4-methylthiazol-2-yl)methyl)isophthalamide: ¹H NMR (300 MHz) 7.51 (s, 1H), 7.41 (s, 1H), 7.38 (s, 1H), 7.18-7.28 (m, 7H), 6.89-6.91 (m, 2H), 6.80-6.83 (m, 1H), 4.95 (m, 2H), 4.39 (m, 1H), 3.73-3.86 (m, 3H), 3.80 (s, 3H), 2.97-3.11 (m, 5H), 2.82 (m, 2H), 2.46 (s, 3H).

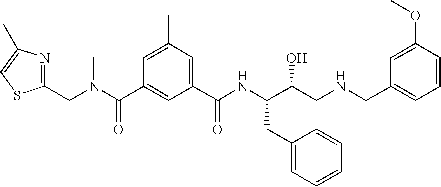

N1-((2S,3R)-3-hydroxy-4-(3-methoxybenzylamino)-1-phenylbutan-2-yl)-N3,5-dimethyl-N3-((4-methylthiazol-2-yl)methyl)isophthalamide: ¹H NMR (300 MHz) 7.44 (s, 2H), 7.32 (s, 1H), 7.16-7.25 (m, 7H), 6.86-6.89 (m, 2H), 6.76-6.79 (m, 1H), 4.94 (m, 2H), 4.36 (m, 1H), 3.71-3.83 (m, 2H), 3.76 (s, 3H), 3.66 (m, 1H), 2.96-3.00 (m, 5H), 2.77 (m, 2H), 2.43 (s, 3H), 2.32 (s, 3H).

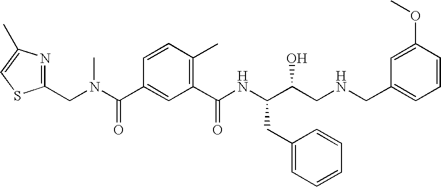

N3-((2S,3R)-3-hydroxy-4-(3-methoxybenzylamino)-1-phenylbutan-2-yl)-N1,4-dimethyl-N1-((4-methylthiazol-2-yl)methyl)isophthalamide

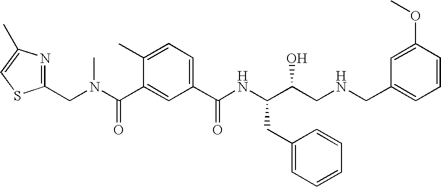

N1-((2S,3R)-3-hydroxy-4-(3-methoxybenzylamino)-1-phenylbutan-2-yl)-N3,4-dimethyl-N3-((4-methylthiazol-2-yl)methyl)isophthalamide: ¹H NMR (300 MHz, CDCl₃) regioisomeric mixture d 7.05-7.45 (m, 9H), 6.87-7.05 (m, 2H), 6.78-6.87 (m, 2H), 4.85-5.05 (m, 2H), 4.30-4.47 (m, 1H), 3.80 (s, 3H), 3.64-3.90 (m, 3H), 3.04-3.28 (m, 3H), 2.90-3.04 (m, 2H), 2.75-2.90 (m, 3H), 2.32-2.53 (m, 3H), 2.16 (s, 3H).

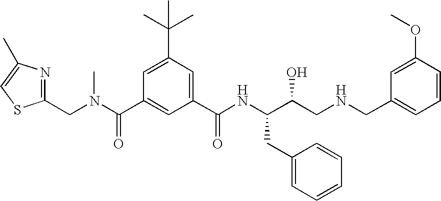

5-tert-butyl-N1-((2S,3R)-3-hydroxy-4-(3-methoxybenzylamino)-1-phenylbutan-2-yl)-N3-methyl-N3-((4-methylthiazol-2-yl)methyl)isophthalamide: ¹H NMR (300 MHz, CDCl₃) δ 7.70-7.45 (m, 4H), 7.30-7.20 (m, 10H), 6.92-6.67 (m, 6H), 4.98 (broad s, 1.3H), 4.62 (broad s, 0.8H), 4.41-4.35 (m, 1H), 3.88-3.67 (m, 7H), 3.15-2.45 (m, 16H), 1.43-1.26 (m, 16H).

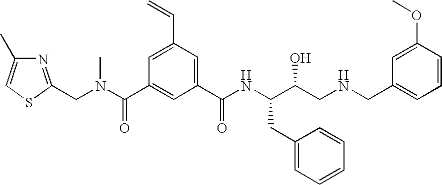

N1-((2S,3R)-3-hydroxy-4-(3-methoxybenzylamino)-1-phenylbutan-2-yl)-N3-methyl-N3-((4-methylthiazol-2-yl)methyl)-5-vinylisophthalamide: ¹H NMR (300 MHz) 7.66 (s, 1H), 7.55 (s, 1H), 7.51 (s, 1H), 7.18-7.29 (m, 7H), 6.79-6.92 (m, 3H), 6.57-6.67 (m, 1H), 5.74 (m, 1H), 5.33 (m, 1H), 4.97 (m, 2H), 4.40 (m, 1H), 3.73-3.81 (m, 3H), 3.78 (s, 3H), 2.99-3.13 (m, 5H), 2.80 (s, 2H), 2.46 (s, 3H).

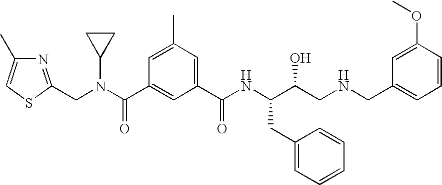

N1-cyclopropyl-N3-((2S,3R)-3-hydroxy-4-(3-methoxybenzylamino)-1-phenylbutan-2-yl)-5-methyl-N1-((4-methylthiazol-2-yl)methyl)isophthalamide: ¹H NMR (300 MHz) 7.64 (s, 1H), 7.54 (m, 2H), 7.31-7.35 (m, 7H), 6.83-6.97 (m, 3H), 5.07 (m, 2H), 4.46 (m, 1H), 3.78-3.95 (m, 3H), 3.88 (s, 3H), 3.11 (m, 2H), 2.88 (m, 2H), 2.54 (s, 3H), 2.45 (s, 3H), 0.54-0.64 (m, 4H).

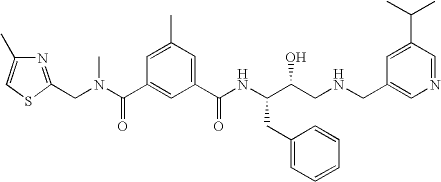

N1-((2S,3R)-3-hydroxy-4-((5-isopropylpyridin-3-yl)methylamino)-1-phenylbutan-2-yl)-N3,5-dimethyl-N3-((4-methylthiazol-2-yl)methyl)isophthalamide: ¹H NMR (300 MHz) 8.42 (m, 2H), 7.58 (m, 1H), 7.49 (m, 2H), 7.25-7.33 (m, 6H), 6.92 (s, 1H), 4.99 (m, 2H), 4.39 (m, 1H), 3.82-3.93 (m, 2H), 3.74 (m, 1H), 2.93-3.14 (m, 6H), 2.84 (m, 2H), 2.48 (s, 3H), 2.39 (s, 3H), 1.27 (m, 6H).

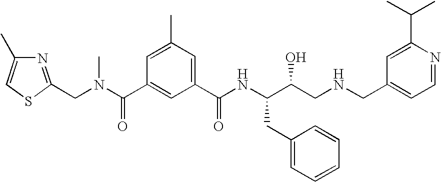

N1-((2S,3R)-3-hydroxy-4-((2-isopropylpyridin-4-yl)methylamino)-1-phenylbutan-2-yl)-N3,5-dimethyl-N3-((4-methylthiazol-2-yl)methyl)isophthalamide: ¹H NMR (300 MHz, CDCl₃) d 8.50-8.52 (m, 1H), 7.42-7.65 (m, 2H), 7.33-7.42 (m, 1H), 7.12-7.33 (m, 8H), 6.87-6.97 (m, 1H), 6.70-6.87 (m, 1H), 4.99 (s, 2H), 4.32-4.45 (m, 1H), 3.85-3.97 (m, 2H), 3.69-3.82 (m, 1H), 2.96-3.19 (m, 6H), 2.79-2.87 (m, 2H), 2.49 (s, 3H), 2.39 (s, 3H), 1.30-1.33 (m, 6H).

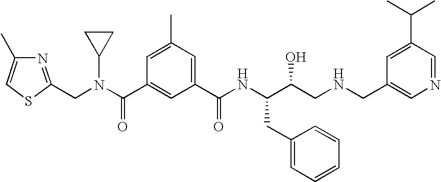

N1-cyclopropyl-N3-((2S,3R)-3-hydroxy-4-((5-isopropylpyridin-3-yl)methylamino)-1-phenylbutan-2-yl)-5-methyl-N1-((4-methylthiazol-2-yl)methyl)isophthalamide: ¹H NMR (300 MHz) 8.43 (m, 2H), 7.57 (m, 2H), 7.50 (m, 2H), 7.24-7.29 (m, 5H), 6.90 (m, 1H), 5.02 (m, 2H), 4.40 (m, 1H), 3.81-3.88 (m, 2H), 3.73 (m, 1H), 2.83-3.10 (m, 5H), 2.49 (s, 3H), 2.41 (s, 3H), 1.28 (m, 6H), 0.05-0.21 (m, 4H).

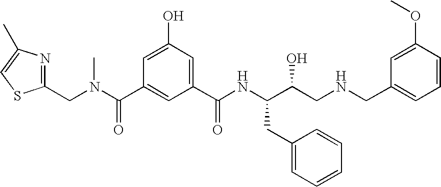

5-hydroxy-N1-((2S,3R)-3-hydroxy-4-(3-methoxybenzylamino)-1-phenylbutan-2-yl)-N3-methyl-N3-((4-methylthiazol-2-yl)methyl)isophthalamide: ¹H NMR (300 MHz, CDCl₃) δ 7.26-7.11 (m, 13H), 6.94-6.76 (m, 5H), 4.89 (broad s, 1.6H), 4.63 (broad s, 0.7H), 4.26-4.23 (m, 1H), 3.86-3.62 (m, 9H), 3.06-2.80 (m, 8H), 2.40 (s, 3H).

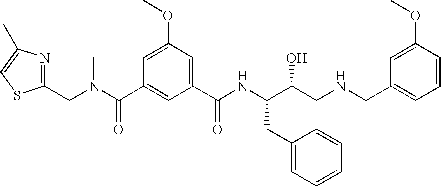

N1-((2S,3R)-3-hydroxy-4-(3-methoxybenzylamino)-1-phenylbutan-2-yl)-5-methoxy-N3-methyl-N3-((4-methylthiazol-2-yl)methyl)isophthalamide: ¹H NMR (300 MHz, CDCl₃) δ 7.32-7.07 (m, 11H), 6.96-6.79 (m, 5H), 4.96 (broad s, 1.2H), 4.64 (broad s, 0.7H), 4.40-4.35 (m, 1H), 3.86-3.66 (m, 9H), 3.11-2.99 (m, 5H), 2.79 (m, 2H), 2.45 (s, 3H).

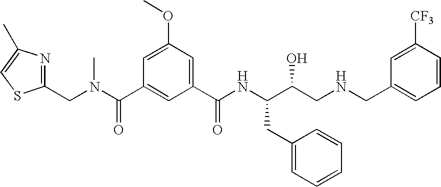

N1-((2S,3R)-3-hydroxy-1-phenyl-4-(3-(trifluoromethyl)benzylamino)butan-2-yl)-5-methoxy-N3-methyl-N3-((4-methylthiazol-2-yl)methyl)isophthalamide: ¹H NMR (300 MHz, CDCl₃) δ 7.57-7.04 (m, 10H), 6.93-6.87 (m, 3H), 4.94 (broad s, 1.3H), 4.62 (broad s, 0.7H), 4.37-4.33 (m, 1H), 3.90-3.69 (m, 6H), 3.10-2.76 (m, 8H), 2.44 (s, 3H).

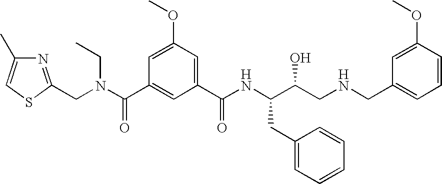

N1-ethyl-N3-((2S,3R)-3-hydroxy-4-(3-methoxybenzylamino)-1-phenylbutan-2-yl)-5-methoxy-N1-((4-methylthiazol-2-yl)methyl)isophthalamide: ¹H NMR (300 MHz, CDCl₃) δ 7.34-7.16 (m, 8H), 7.07-6.85 (m, 4H), 5.01 (broad s, 1.2H), 4.68 (broad s, 0.8H), 4.43-4.41 (m, 1H), 3.87-3.35 (m, 11H), 3.06-2.84 (m, 6H), 2.50 (s, 3H), 1.33-1.17 (m, 5H).

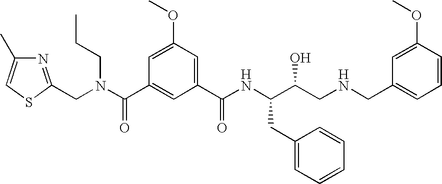

N1-((2S,3R)-3-hydroxy-4-(3-methoxybenzylamino)-1-phenylbutan-2-yl)-5-methoxy-N3-((4-methylthiazol-2-yl)methyl)-N3-propylisophthalamide: ¹H NMR (300 MHz, CDCl₃) δ 7.26-7.14 (m, 12H), 6.91-6.78 (m, 4H), 4.95 (broad s, 1.4H), 4.62 (broad s, 0.8H), 4.38-4.36 (m, 1H), 3.81-3.68 (m, 11H), 3.20-2.78 (m, 10H), 2.44 (s, 3H), 1.55 (m, 3H), 0.88-0.71 (m, 5H).

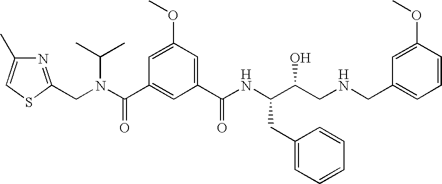

N1-((2S,3R)-3-hydroxy-4-(3-methoxybenzylamino)-1-phenylbutan-2-yl)-N3-isopropyl-5-methoxy-N3-((4-methylthiazol-2-yl)methyl)isophthalamide: ¹H NMR (300 MHz, CDCl₃) δ 7.26-7.16 (m, 8H), 7.00-6.78 (m, 5H), 4.88 (broad s, 1.6H), 4.60 (broad s, 0.3H), 4.38-4.34 (m, 1H), 3.94-3.68 (m, 9H), 3.00-2.78 (m, 5H), 2.43 (broad s, 3H), 1.25-1.14 (m, 9H).

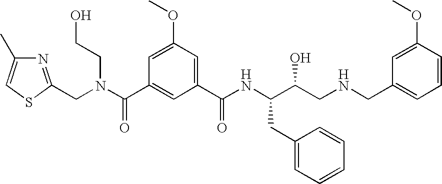

N1-((2S,3R)-3-hydroxy-4-(3-methoxybenzylamino)-1-phenylbutan-2-yl)-N3-(2-hydroxyethyl)-5-methoxy-N3-((4-methylthiazol-2-yl)methyl)isophthalamide: ¹H NMR (300 MHz, CDCl₃) δ 7.46 (s, 1H), 7.31-7.06 (m, 9H), 6.93-6.81 (m, 4H), 4.90 (broad s, 1.4H), 4.73 (broad s, 0.4H), 4.42-4.37 (m, 1H), 3.91-3.74 (m, 11H), 3.56 (m, 2H), 3.09-2.81 (m, 5H), 2.45 (s, 3H).

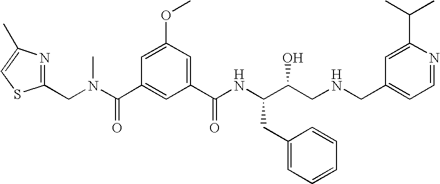

N1-((2S,3R)-3-hydroxy-4-((2-isopropylpyridin-4-yl)methylamino)-1-phenylbutan-2-yl)-5-methoxy-N3-methyl-N3-((4-methylthiazol-2-yl)methyl)isophthalamide: ¹H NMR (300 MHz, CDCl₃) d 8.48-8.50 (m, 1H), 7.05-7.37 (m, 11H), 6.85-6.96 (m, 2H), 4.98 (s, 2H), 4.34-4.46 (m, 1H), 3.84 (s, 3H), 3.64-3.91 (m, 3H), 2.96-3.20 (m, 6H), 2.73-2.88 (m, 2H), 2.48 (s, 3H), 1.30-1.32 (m, 6H).

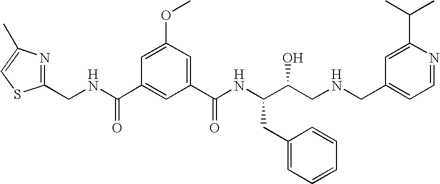

N1-((2S,3R)-3-hydroxy-4-((2-isopropylpyridin-4-yl)methylamino)-1-phenylbutan-2-yl)-5-methoxy-N3-((4-methylthiazol-2-yl)methyl)isophthalamide: ¹H NMR (300 MHz, CDCl₃) d 8.49-8.50 (m, 1H), 7.92-8.04 (m, 1H), 7.68 (s, 1H), 7.49 (s, 1H), 7.10-7.30 (m, 11H), 6.85 (s, 1H), 4.88-4.90 (m, 2H), 4.34-4.47 (m, 1H), 3.81 (s, 3H), 3.74-3.97 (m, 3H), 2.97-3.10 (m, 3H), 2.81-2.90 (m, 2H), 2.40 (s, 3H), 1.27-1.30 (m, 6H).

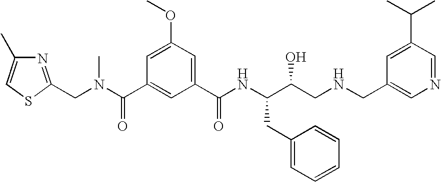

N1-((2S,3R)-3-hydroxy-4-((5-isopropylpyridin-3-yl)methylamino)-1-phenylbutan-2-yl)-5-methoxy-N3-methyl-N3-((4-methylthiazol-2-yl)methyl)isophthalamide: ¹H NMR (300 MHz, CDCl₃) δ 8.38 (s, 2H), 7.55-7.05 (m, 13H), 6.88 (m, 2H), 4.95 (broad s, 1.3H), 4.64 (broad s, 0.6H), 4.35 (m, 1H), 3.81-3.71 (m, 7H), 3.08-2.78 (m, 9H), 2.44 (s, 4H), 1.24 (d, 12H).

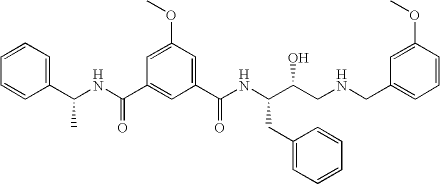

N1-((2S,3R)-3-hydroxy-4-(3-methoxybenzylamino)-1-phenylbutan-2-yl)-5-methoxy-N3-((R)-1-phenylethyl)isophthalamide: ¹H NMR (300 MHz, CDCl₃) δ 7.58 (m, 1H), 7.46-7.18 (m, 14H), 6.92-6.69 (m, 4H), 5.36-5.31 (m, 1H), 4.43-4.39 (m, 1.0H), 3.86-3.69 (m, 9H), 2.99-2.97 (m, 2H), 2.82-2.81 (m, 2H), 1.62 (d, 3H).

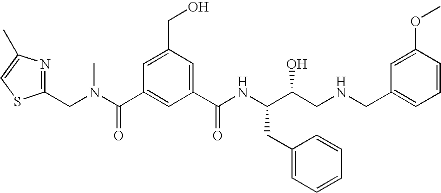

N1-((2S,3R)-3-hydroxy-4-(3-methoxybenzylamino)-1-phenylbutan-2-yl)-5-(hydroxymethyl)-N3-methyl-N3-((4-methylthiazol-2-yl)methyl)isophthalamide: ¹H NMR (300 MHz, CDCl₃+CD₃OD), d: 7.654-7.508 (m, 3H), 7.300-7.135 (m, 6H), 6.913-6.798 (m 4H), 4.950 (br, 1.4H), 4.614 (br, 2.6H), 4.317 (m, 1H), 3.866-3.737 (m, 6H), 3.120-2.790 (m, 7H), 2.450 (s, 3H).

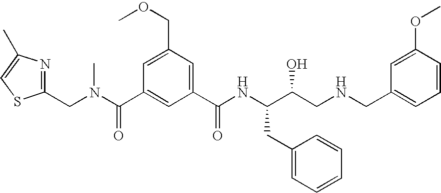

N1-((2S,3R)-3-hydroxy-4-(3-methoxybenzylamino)-1-phenylbutan-2-yl)-5-(methoxymethyl)-N3-methyl-N3-((4-methylthiazol-2-yl)methyl)isophthalamide: ¹H NMR (300 MHz, CDCl₃+CD₃OD), d: 7.665-7.521 (m, 2H), 7.253-7.127 (m, 6H), 6.897-6.775 (m, 4H), 4.938 (br, 1.4H), 4.646 (br, 0.6H), 4.450 (s, 2H), 4.325 (m, 1H), 3.837-3.3.709 (m, 3 H), 3.772 (s, 3H), 3.390 (s, 3H), 3.099-2.766 (m, 7H), 2.438 (s, 3H).

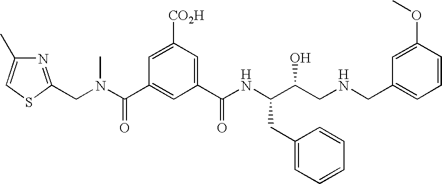

3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzoic acid: ¹H NMR (300 MHz) 8.38 (m, 2H), 8.21 (m, 1H), 7.89 (m, 1H), 7.21-7.31 (m, 6H), 7.00-7.05 (m, 1H), 6.90-6.94 (m, 2H), 4.97 (m, 2H), 4.28 (m, 1H), 4.03-4.07 (m, 2H), 3.81 (s, 3H), 3.17-3.28 (m, 2H), 3.00 (s, 3H), 2.92-2.99 (m, 2H), 2.41 (s, 3H).

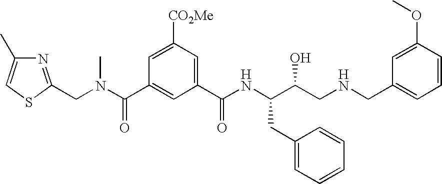

methyl 3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzoate: ¹H NMR 8.36 (s, 2H), 8.20 (s, 1H), 8.00 (s, 1H), 7.18-7.29 (m, 6H), 6.79-6.96 (m, 3H), 5.01 (m, 2H), 4.44 (m, 1H), 3.92 (s, 3H), 3.69-3.88 (m, 6H), 2.96-3.19 (m, 5H), 2.83 (s, 2H), 2.43 (s, 3H).

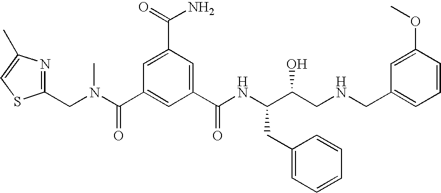

N1-((2S,3R)-3-hydroxy-4-(3-methoxybenzylamino)-1-phenylbutan-2-yl)-N3-methyl-N3-((4-methylthiazol-2-yl)methyl)benzene-1,3,5-tricarboxamide: ¹H NMR (300 MHz, CDCl₃) δ 8.29-7.87 (m, 4H), 7.31-7.18 (m, 12H), 7.02-6.81 (m, 5H), 5.97 (broad s, 1H), 4.98 (broad s, 1.3H), 4.63 (broad s, 0.7H), 4.46-4.41 (m, 1H), 3.93-3.68 (m, 7H), 3.16-2.86 (m, 8H), 2.48 (s, 3H).

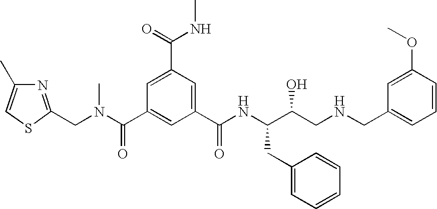

N1-((2S,3R)-3-hydroxy-4-(3-methoxybenzylamino)-1-phenylbutan-2-yl)-N3,N5-dimethyl-N3-((4-methylthiazol-2-yl)methyl)benzene-1,3,5-tricarboxamide: ¹H NMR (300 MHz, CDCl₃) δ 8.04-7.79 (m, 4H), 7.32-7.18 (m, 7H), 7.07 (broad s, 1H), 6.94-6.80 (m, 4H), 4.99 (broad s, 1.3H), 4.61 (broad s, 0.6H), 4.45-4.41 (m, 1H), 3.94-3.80 (m, 6H), 3.15-2.86 (m, 10H), 2.48 (s, 3H).

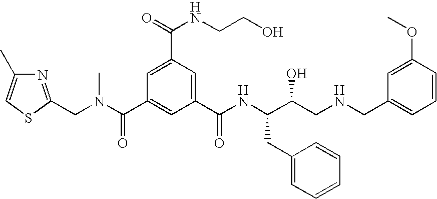

N1-((2S,3R)-3-hydroxy-4-(3-methoxybenzylamino)-1-phenylbutan-2-yl)-N3-(2-hydroxyethyl)-N5-methyl-N5-((4-methylthiazol-2-yl)methyl)benzene-1,3,5-tricarboxamide: ¹H NMR (300 MHz) 8.11 (s, 1H), 7.88 (s, 1H), 7.64 (s, 1H), 7.13-7.29 (m, 7H), 6.83-6.99 (m, 3H), 4.96 (m, 2H), 4.32 (m, 1H), 3.94-3.97 (m, 2H), 3.82-3.84 (m, 3H), 3.76 (s, 3H), 3.56 (m, 2H), 2.81-3.12 (m, 7H), 2.47 (s, 3H).

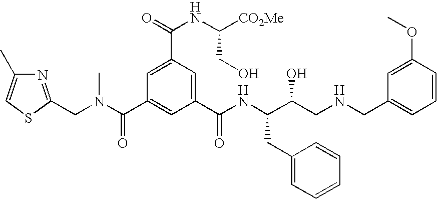

(S)-methyl 3-hydroxy-2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzamido)propanoate: ¹H NMR (300 MHz) 8.10 (m, 1H), 7.96 (m, 2H), 7.62 (m, 1H), 7.13-7.332 (m, 6H), 6.92-6.97 (m, 2H), 6.80-6.84 (m, 1H), 4.90-5.08 (m, 2H), 4.81 (m, 1H), 4.33 (m, 1H), 4.14-4.19 (m, 2H), 3.78-3.98 (m, 3H), 3.80 (s, 3H), 3.77 (s, 3H), 2.91-3.12 (m, 4H), 2.87 (s, 3H), 2.48 (s, 3H).

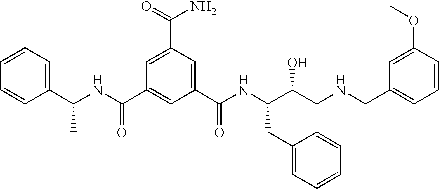

N1-((2S,3R)-3-hydroxy-4-(3-methoxybenzylamino)-1-phenylbutan-2-yl)-N3-((R)-1-phenylethyl)benzene-1,3,5-tricarboxamide: ¹H NMR (300 MHz, CDCl₃+CD₃OD) δ 8.35-8.26 (m, 3H), 7.39-7.10 (m, 11H), 6.88-6.77 (m, 3H), 5.30-5.23 (m, 1H), 4.38-4.31 (m, 1.0H), 3.84-3.71 (m, 6H), 3.08-2.76 (m, 4H), 1.61 (d, 3H).

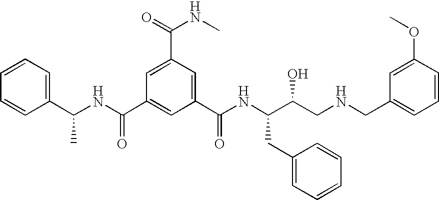

N1-((2S,3R)-3-hydroxy-4-(3-methoxybenzylamino)-1-phenylbutan-2-yl)-N3-methyl-N5-((R)-1-phenylethyl)benzene-1,3,5-tricarboxamide: ¹H NMR (300 MHz, CDCl₃) δ 8.19-8.07 (m, 3H), 7.88 (m, 1H), 7.41-7.06 (m, 13H), 6.91-6.79 (m, 4H), 5.35-5.25 (m, 1H), 4.45-4.39 (m, 1H), 3.90-3.61 (m, 6H), 2.99-2.75 (m, 7H), 1.62 (d, 3H).

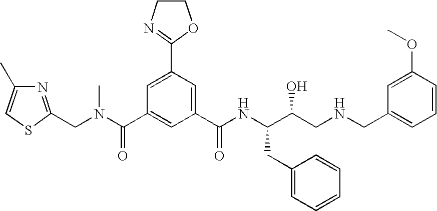

5-(4,5-dihydrooxazol-2-yl)-N1-((2S,3R)-3-hydroxy-4-(3-methoxybenzylamino)-1-phenylbutan-2-yl)-N3-methyl-N3-((4-methylthiazol-2-yl)methyl)isophthalamide: ¹H NMR (300 MHz) 8.26 (s, 1H), 8.15 (s, 1H), 7.89 (s, 1H), 7.20-7.29 (m, 7H), 6.93-6.95 (m, 2H), 6.81-6.84 (m, 1H), 4.99 (m, 2H), 4.41-4.51 (m, 3H), 4.05-4.12 (m, 2H), 3.77-3.88 (m, 6H), 2.96-3.15 (m, 5H), 2.86 (m, 2H), 2.48 (s, 3H).

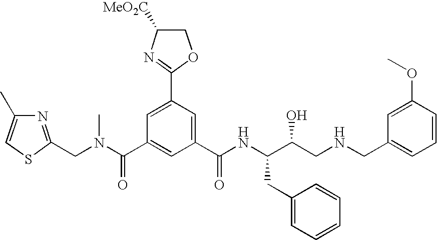

(S)-methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)-4,5-dihydrooxazole-4-carboxylate: ¹H NMR (300 MHz) 8.58 (s, 1H), 8.22 (s, 2H), 7.21-7.29 (m, 7H), 6.79-6.93 (m, 3H), 4.98-5.04 (m, 2H), 4.65-4.75 (m, 3H), 4.53 (m, 1H), 4.27 (m, 1H), 3.85 (s, 3H), 3.81 (s, 3H), 3.67-3.76 (m, 2H), 3.16-3.29 (m, 2H), 3.06 (s, 3H), 2.52-2.78 (m, 2H), 2.48 (s, 3H).

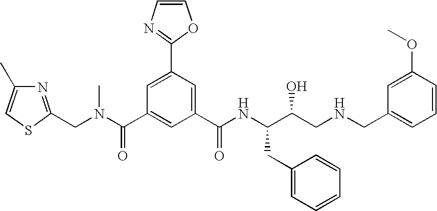

N1-((2S,3R)-3-hydroxy-4-(3-methoxybenzylamino)-1-phenylbutan-2-yl)-N3-methyl-N3-((4-methylthiazol-2-yl)methyl)-5-(oxazol-2-yl)isophthalamide: ¹H NMR (300 MHz) 8.32 (s, 1H), 8.20 (s, 1H), 7.81 (s, 1H), 7.73 (s, 2H), 7.18-7.30 (m, 7H), 6.90-6.93 (m, 2H), 6.76-6.80 (m, 1H), 4.99 (m, 2H), 4.43 (m, 1H), 3.79-3.85 (m, 3H), 3.78 (s, 3H), 3.00-3.16 (m, 5H), 2.85 (m, 2H), 2.48 (s, 3H).

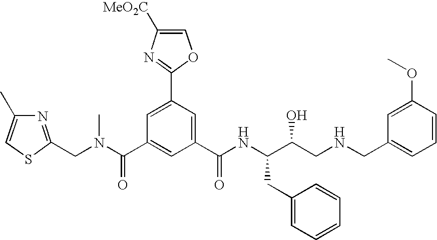

methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)oxazole-4-carboxylate: ¹H NMR (300 MHz) 8.73 (s, 1H), 8.35 (s, 2H), 8.20 (s, 1H), 7.21-7.31 (m, 7H), 6.79-6.94 (m, 3H), 5.01 (m, 2H), 4.56 (m, 1H), 4.32 (m, 1H), 3.99 (s, 3H), 3.82 (s, 3H), 3.69-3.77 (m, 2H), 3.18-3.30 (m, 2H), 3.10 (s, 3H), 2.55-2.81 (m, 2H), 2.49 (s, 3H).

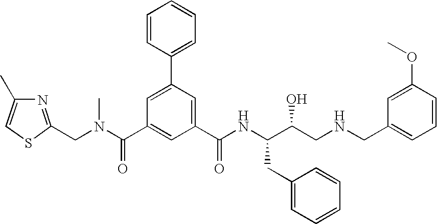

N3-((2S,3R)-3-hydroxy-4-(3-methoxybenzylamino)-1-phenylbutan-2-yl)-N5-methyl-N5-((4-methylthiazol-2-yl)methyl)biphenyl-3,5-dicarboxamide: ¹H NMR (300 MHz, CDCl₃): 2.48 (s, 3H), 2.81-2.82 (m, 2H), 3.04-3.18 (m, 5H), 3.71-3.87 (m, 6H), 4.38-4.49 (m, 1H), 4.70 (br s, 1H), 5.01 (br s, 1H), 6.78-6.81 (m, 1H), 6.89-6.92 (m, 2H), 7.20-7.30 (m, 7H), 7.42-7.56 (m, 5H), 7.66 (s, 1H), 7.75 (s, 1H), 7.89 (br s, 1H).

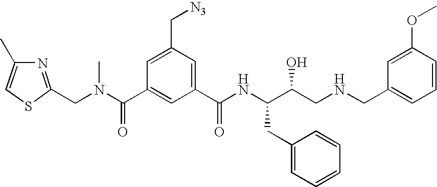

5-(azidomethyl)-N1-((2S,3R)-3-hydroxy-4-(3-methoxybenzylamino)-1-phenylbutan-2-yl)-N3-methyl-N3-((4-methylthiazol-2-yl)methyl)isophthalamide: ¹H NMR (300 MHz, CDCl₃+CD₃OD), d: 7.684-7.510 (m, 3H), 7.301-7.157 (m, 6H), 6.914-6.798 (m, 4H), 4.964 (br, 1.4H), 4.650 (br, 0.6H), 4.370 (s, 3H), 3.854-3.725 (m, 6H), 3.057-2.896 (m, 5H), 2.819 (s, 2H), 2.460 (s, 3H).

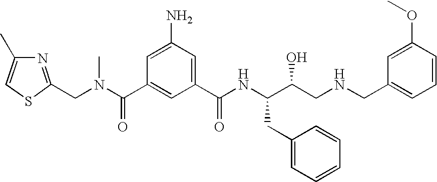

5-amino-N1-((2S,3R)-3-hydroxy-4-(3-methoxybenzylamino)-1-phenylbutan-2-yl)-N3-methyl-N3-((4-methylthiazol-2-yl)methyl)isophthalamide: ¹H NMR (300 MHz, CDCl₃): 2.32-2.48 (m, 2H), 2.38 (s, 3H), 2.81-3.21 (m, 5H), 3.61-4.12 (m, 6H), 4.32-4.44 (m, 1H), 4.82 (s, 1H), 4.88-5.02 (s, 1H), 6.65-6.83 (m, 4H), 7.12-7.39 (m, 9H).

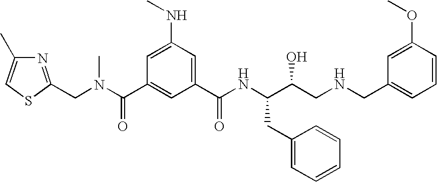

N1-((2S,3R)-3-hydroxy-4-(3-methoxybenzylamino)-1-phenylbutan-2-yl)-N3-methyl-5-(methylamino)-N3-((4-methylthiazol-2-yl)methyl)isophthalamide: ¹H NMR (300 MHz, CDCl₃) δ 7.32-7.19 (m, 11H), 6.96-6.72 (m, 9H), 4.98 (broad s, 1.4H), 4.69 (broad s, 0.8H), 4.41-4.37 (m, 1H), 3.95-3.68 (m, 8H), 3.12-2.85 (m, 11H), 2.48 (s, 3H).

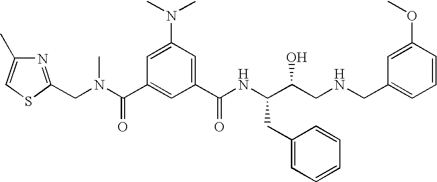

5-(dimethylamino)-N1-((2S,3R)-3-hydroxy-4-(3-methoxybenzylamino)-1-phenylbutan-2-yl)-N3-methyl-N3-((4-methylthiazol-2-yl)methyl)isophthalamide: ¹H NMR (300 MHz, CDCl₃) δ 7.34-7.19 (m, 7H), 7.04-6.80 (m, 6H), 6.62-6.54 (m, 1H), 4.99 (broad s, 1.0H), 4.68 (broad s, 0.8H), 4.41-4.36 (m, 1H), 3.88-3.67 (m, 6H), 3.14-3.00 (m, 11H), 2.81-2.79 (m, 2H), 2.48 (s, 3H).

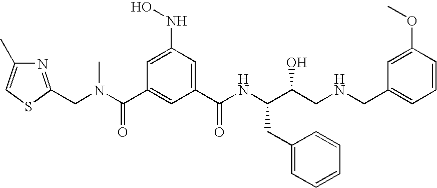

N1-((2S,3R)-3-hydroxy-4-(3-methoxybenzylamino)-1-phenylbutan-2-yl)-5-(hydroxyamino)-N3-methyl-N3-((4-methylthiazol-2-yl)methyl)isophthalamide: ¹H NMR (300 MHz, CDCl₃) δ 8.14-8.11 (m, 1H), 7.35-6.78 (m, 11H), 5.02-4.89 (m, 1.2H), 4.61 (broad s, 0.6H), 4.36-4.32 (m, 1H), 3.92-3.53 (m, 8H), 3.11 (s, 1H), 2.95-2.82 (m, 5H), 2.47-2.42 (m, 3H).

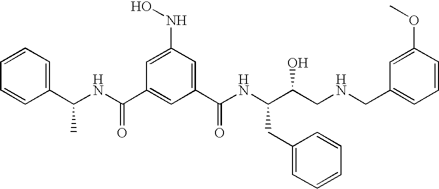

N1-((2S,3R)-3-hydroxy-4-(3-methoxybenzylamino)-1-phenylbutan-2-yl)-5-(hydroxyamino)-N3-((R)-1-phenylethyl)isophthalamide: ¹H NMR (300 MHz, CDCl₃) δ 7.80 (m, 1H), 7.37-7.10 (m, 13H), 7.00 (m, 2H), 6.89-6.79 (m, 3H), 5.25-5.20 (m, 1H), 4.33 (m, 1H), 3.85-3.65 (m, 6H), 2.92-2.80 (m, 4H), 1.55 (d, 3H).

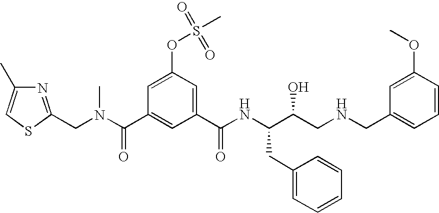

3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl methanesulfonate: ¹H NMR (300 MHz, CDCl₃) δ 7.83-7.22 (m, 11H), 6.94-6.82 (m, 4H), 4.98 (broad s, 1.3H), 4.66 (broad s, 0.6H), 4.44-4.39 (m, 1H), 3.91-3.72 (m, 6H), 3.23-2.83 (m, 10H), 2.48 (s, 4H).

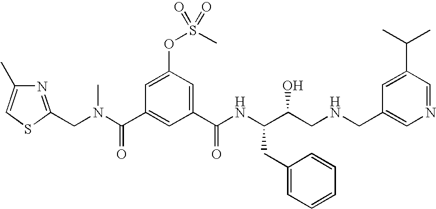

3-((2S,3R)-3-hydroxy-4-((5-isopropylpyridin-3-yl)methylamino)-1-phenylbutan-2-ylcarbamoyl)-5-(methyl((4-methylthiazol-2-yl)methyl)carbamoyl)phenyl methanesulfonate: ¹H NMR (300 MHz, CDCl₃) δ 8.40 (broad s, 2H), 7.74-7.44 (m, 4H), 7.31-7.18 (m, 6H), 6.92 (broad s, 1H), 4.98 (broad s, 1.3H), 4.67 (broad s, 0.6H), 4.43-4.38 (m, 1H), 3.92-3.73 (m, 3H), 3.22-2.83 (m, 11H), 2.48 (s, 3H), 1.29-1.26 (m, 10H).

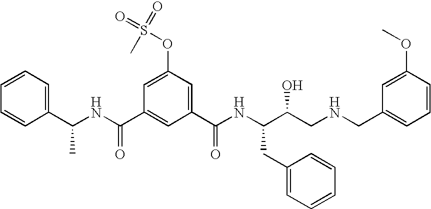

3-((2S,3R)-3-hydroxy-4-(3-methoxybenzylamino)-1-phenylbutan-2-ylcarbamoyl)-5-((R)-1-phenylethylcarbamoyl)phenyl methanesulfonate: ¹H NMR (300 MHz, CDCl₃) δ 8.07 (s, 1H), 7.87-7.80 (m, 2H), 7.64 (s, 1H), 7.40-7.17 (m, 10H), 7.03-6.81 (m, 3H), 5.33-5.28 (m, 1H), 4.41-4.37 (m, 1.0H), 3.89-3.73 (m, 6H), 3.14-3.13 (m, 3H), 2.97-2.77 (m, 4H), 1.62 (d, 3H).

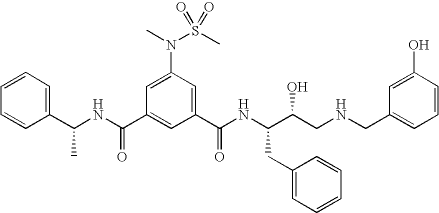

N1-((2S,3R)-3-hydroxy-4-(3-hydroxybenzylamino)-1-phenylbutan-2-yl)-5-(N-methylmethylsulfonamido)-N3-((R)-1-phenylethyl)isophthalamide: ¹H NMR (300 MHz) 8.12 (m, 1H), 7.90 (m, 1H), 7.76 (m, 1H), 7.44 (m, 2H), 7.08-7.34 (m, 9H), 6.91 (m, 1H), 6.72-6.75 (m, 2H), 5.30 (m, 1H), 4.30 (m, 1H), 3.74-3.95 (m, 3H), 3.26 (s, 3H), 3.05-3.12 (m, 1H), 2.86-2.90 (m, 3H), 2.82 (s, 3H).

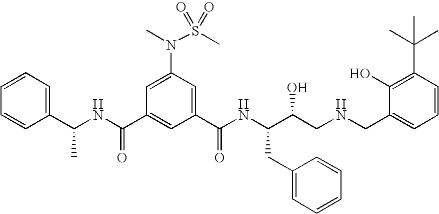

N1-((2S,3R)-4-(3-tert-butyl-2-hydroxybenzylamino)-3-hydroxy-1-phenylbutan-2-yl)-5-(N-methylmethylsulfonamido)-N3-((R)-1-phenylethyl)isophthalamide: ¹H NMR (300 MHz) 7.91 (m, 1H), 7.83 (m, 1H), 7.69 (m, 1H), 7.37-7.44 (m, 3H), 7.20-7.31 (m, 7H), 6.90-6.93 (m, 1H), 6.75-6.80 (m, 2H), 5.31 (m, 1H), 4.34 (m, 1H), 3.96-4.12 (m, 3H), 3.30 (s, 3H), 2.83-3.01 (m, 4H), 2.92 (s, 3H), 1.65 (m, 3H), 1.44 (s, 9H).

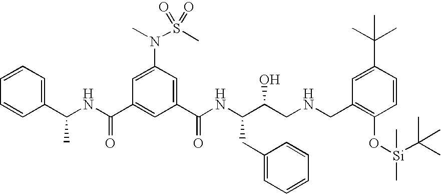

N1-((2S,3R)-4-(5-tert-butyl-2-(tert-butyldimethylsilyloxy)benzylamino)-3-hydroxy-1-phenylbutan-2-yl)-5-(N-methylmethylsulfonamido)-N3-((R)-1-phenylethyl)isophthalamide: ¹H NMR (300 MHz) 7.93 (m, 1H), 7.85 (m, 1H), 7.71 (m, 1H), 7.21-7.28 (m, 3H), 7.03-7.16 (m, 9H), 6.63 (m, 1H), 5.20 (m, 1H), 4.28 (m, 1H), 3.58-3.75 (m, 3H), 3.20 (s, 3H), 2.63-2.84 (m, 4H), 2.70 (s, 3H), 1.50 (m, 3H), 1.16 (s, 9H), 0.88 (s, 9H), 0.14 (m, 6H).

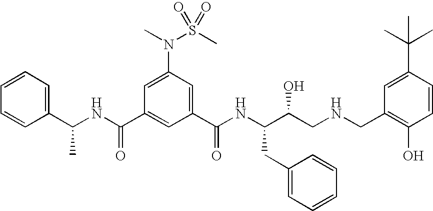

N1-((2S,3R)-4-(5-tert-butyl-2-hydroxybenzylamino)-3-hydroxy-1-phenylbutan-2-yl)-5-(N-methylmethylsulfonamido)-N3-((R)-1-phenylethyl)isophthalamide: ¹H NMR (300 MHz) 8.02 (m, 1H), 7.96 (m, 1H), 7.80 (m, 1H), 7.23-7.40 (m, 11H), 7.05 (m, 1H), 6.82 (m, 1H), 5.33 (m, 1H), 4.38 (m, 1H), 3.90-4.07 (m, 3H), 3.33 (s, 3H), 2.81-2.97 (m, 4H), 2.86 (s, 3H), 1.65 (m, 3H), 1.30 (s, 9H).

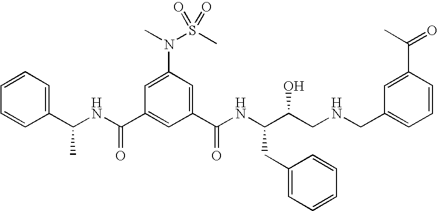

N1-((2S,3R)-4-(3-acetylbenzylamino)-3-hydroxy-1-phenylbutan-2-yl)-5-(N-methylmethylsulfonamido)-N3-((R)-1-phenylethyl)isophthalamide: ¹H NMR (300 MHz) 8.08 (s, 1H), 7.95 (s, 2H), 7.83 (m, 1H), 7.79 (s, 1H), 7.61 (m, 1H), 7.54 (m, 1H), 7.19-7.46 (m, 10H), 5.32 (m, 1H), 4.37 (m, 1H), 3.81-3.97 (m, 2H), 3.72 (m, 1H), 3.29 (s, 3H), 2.90-2.93 (m, 2H), 2.79-2.83 (m, 2H), 2.81 (s, 3H), 2.58 (s, 3H), 1.64 (m, 3H).

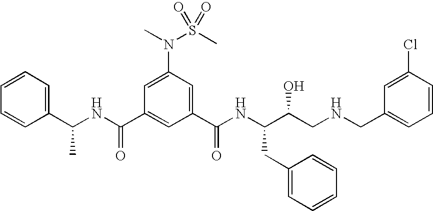

N1-((2S,3R)-4-(3-chlorobenzylamino)-3-hydroxy-1-phenylbutan-2-yl)-5-(N-methylmethylsulfonamido)-N3-((R)-1-phenylethyl)isophthalamide: ¹H NMR (300 MHz, CDCl₃+CD₃OD), d: 8.071 (s, 1H), 7.937 (s, 1H), 7.806 (s, 1H), 7.419-7.215 (m, 14H), 5.318 (m, 1H), 4.374 (m 1H), 3.878-3.748 (m, 3H), 3.322 (s, 3H), 2.999-2.790 (m, 4H), 2.846 (s, 3H), 1.624 (d, J=6.9 Hz, 3H).

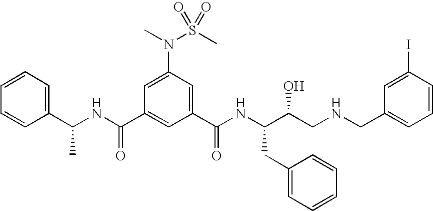

N1-((2S,3R)-3-hydroxy-4-(3-iodobenzylamino)-1-phenylbutan-2-yl)-5-(N-methylmethylsulfonamido)-N3-((R)-1-phenylethyl)isophthalamide: ¹H NMR (300 MHz, CDCl₃+CD₃OD), d: 8.076-7.574 (m, 6H), 7.411-7.033 (m, 11H), 5.302 (m, 1H), 4.369 (m 1 H), 3.817-3.683 (m, 3H), 3.306 (s, 3H), 2.968-2.764 (m, 4H), 2.848 (s, 3H), 1.605 (d, J=6.9 Hz, 3H).

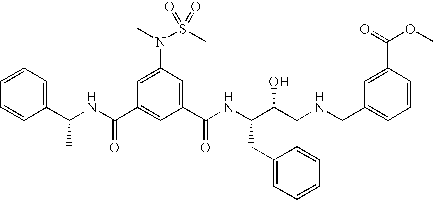

methyl 3-(((2R,3S)-2-hydroxy-3-(3-(N-methylmethylsulfonamido)-5-((R)-1-phenylethylcarbamoyl)benzamido)-4-phenylbutylamino)methyl)benzoate: ¹H NMR (300 MHz, CDCl₃+CD₃OD), d: 8.067-7.769 (m, 5H), 7.533-7.169 (m, 12H), 5.283 (m, 1H), 4.366 (m 1H), 3.899 (s, 3H), 3.899-3.726 (m, 3H), 3.237 (s, 3H), 2.943-2.770 (m, 4H), 2.770 (s, 3H), 1.560 (d, J=6.9 Hz, 3H).

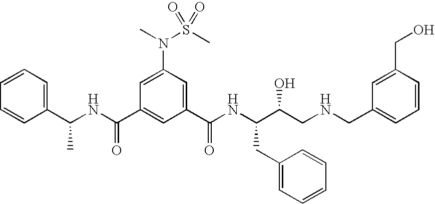

N1-((2S,3R)-3-hydroxy-4-(3-(hydroxymethyl)benzylamino)-1-phenylbutan-2-yl)-5-(N-methylmethylsulfonamido)-N3-((R)-1-phenylethyl)isophthalamide: ¹H NMR (300 MHz, CDCl₃+CD₃OD), d: 8.110 (s, 1H), 7.912 (s, 1H), 7.761 (s, 1H), 7.431-7.127 (m, 14H), 5.283 (m, 1H), 4.567 (m 2H), 4.264 (m, 1H), 3.880 (m, 3H), 3.284 (s, 3H), 3.092 (m, 1H), 2.908-2.796 (m, 6H), 1.619 (d, J=6.9 Hz, 3H).

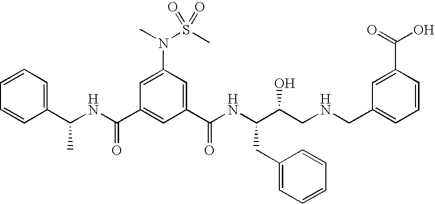

3-(((2R,3S)-2-hydroxy-3-(3-(N-methylmethylsulfonamido)-5-((R)-1-phenylethylcarbamoyl)benzamido)-4-phenylbutylamino)methyl)benzoic acid: ¹H NMR (300 MHz, CDCl₃+CD₃OD), d: 8.242 (s, 2H), 8.102 (s, 1H), 7.841 (s, 2H), 7.727 (s, 1H), 7.383-7.084 (m, 11H), 5.197 (br, 1H), 4.148 (br, 3H), 3.631-3.485 (m, 2H), 3.335-3.011 (m, 2H), 3.176 (s, 3H), 2.876 (m, 1H), 2.761 (s, 3H), 1.537 (m, 3H).

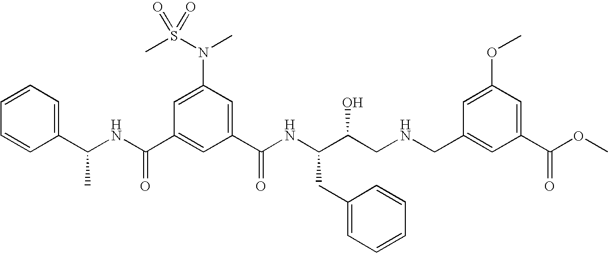

methyl 3-(((2R,3S)-2-hydroxy-3-(3-(N-methylmethylsulfonamido)-5-((R)-1-phenylethylcarbamoyl)benzamido)-4-phenylbutylamino)methyl)-5-methoxybenzoate: ¹H NMR (300 MHz, CDCl₃+CD₃OD), d: 8.060-7.580 (m, 4H), 7.448-7.084 (m, 1H), 5.296 (m, 1H), 4.359 (m 1H), 3.889 (s, 3H), 3.817 (s, 3H), 3.337-3.733 (m, 3H), 3.295 (s, 3H), 3.013-2.764 (m, 4H), 2.844 (s, 3H), 1.593 (d, J=6.9 Hz, 3H).

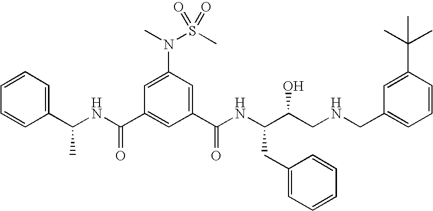

N1-((2S,3R)-4-(3-tert-butylbenzylamino)-3-hydroxy-1-phenylbutan-2-yl)-5-(N-methylmethylsulfonamido)-N3-((R)-1-phenylethyl)isophthalamide: ¹H NMR (300 MHz, CDCl₃): 1.31 (s, 9H), 1.62 (d, 3H, J=6.6 Hz), 2.81-2.99 (m, 7H), 3.31 (s, 3H), 3.71-3.94 (m, 3H), 4.37-4.44 (m, 1H), 5.28-5.38 (m, 1H), 7.14-7.41 (m, 14H), 7.84-7.85 (m, 1H), 7.95-7.96 (m, 1H), 8.07 (s, 1H).

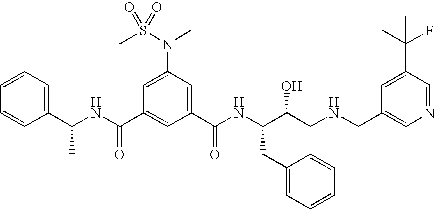

N1-((2S,3R)-4-((5-(2-fluoropropan-2-yl)pyridin-3-yl)methylamino)-3-hydroxy-1-phenylbutan-2-yl)-5-(N-methylmethylsulfonamido)-N3-((R)-1-phenylethyl)isophthalamide: ¹H NMR (300 MHz, CDCl₃+CD₃OD), d: 8.450 (m, 2H), 8.086-7.712 (m, 4H), 7.419-7.138 (m, 10H), 5.314 (m, 1H), 4.340 (m 1H), 3.959-3.718 (m, 3H), 3.314 (s, 3H), 3.050 (m, 1 H), 2.878 (m, 1H), 2.866 (s, 3H), 2.745 (m, 2H), 1.709 (s, 3H), 1.635 (s, 3H), 1.614 (d, J=4.8 Hz, 3H).

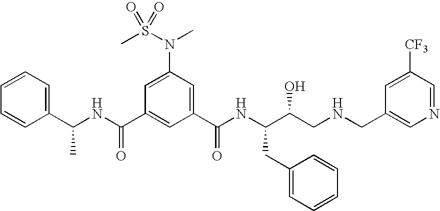

N1-((2S,3R)-3-hydroxy-1-phenyl-4-((5-(trifluoromethyl)pyridin-3-yl)methylamino)butan-2-yl)-5-(N-methylmethylsulfonamido)-N3-((R)-1-phenylethyl)isophthalamide: ¹H NMR (300 MHz, CDCl₃+CD₃OD), d: 8.748 (m, 2H), 8.066-7.750 (m, 4H), 7.421-7.148 (m, 10H), 5.316 (m, 1H), 4.347 (m 1H), 4.017-3.731 (m, 3H), 3.322 (s, 3H), 3.083 (m, 1H), 2.901 (m, 1H), 2.868 (s, 3H), 2.745 (m, 2H), 1.622 (d, J=6.9 Hz, 3H).

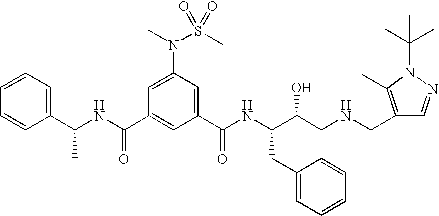

N1-((2S,3R)-4-((1-tert-butyl-5-methyl-1H-pyrazol-4-yl)methylamino)-3-hydroxy-1-phenylbutan-2-yl)-5-(N-methylmethylsulfonamido)-N3-((R)-1-phenylethyl)isophthalamide: ¹H NMR (300 MHz) 8.18 (m, 1H), 7.93 (m, 1H), 7.82 (m, 1H), 7.18-7.44 (m, 11H), 5.30 (m, 1H), 4.35 (m, 1H), 3.63-3.81 (m, 3H), 3.30 (s, 3H), 2.79-2.90 (m, 4H), 2.78 (s, 3H), 2.39 (s, 3H), 1.64 (m, 3H), 1.60 (s, 9H).

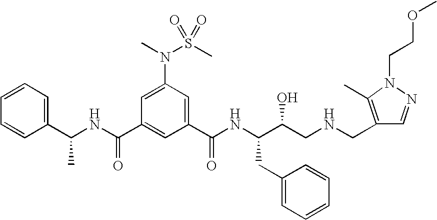

N1-((2S,3R)-3-hydroxy-4-((1-(2-methoxyethyl)-5-methyl-1H-pyrazol-4-yl)methylamino)-1-phenylbutan-2-yl)-5-(N-methylmethylsulfonamido)-N3-((R)-1-phenylethyl)isophthalamide: ¹H NMR (300 MHz) 8.13 (s, 1H), 7.95 (s, 1H), 7.79 (s, 1H), 7.43-7.50 (m, 3H), 7.23-7.35 (m, 8H), 5.31 (m, 1H), 4.33 (m, 1H), 4.12 (m, 2H), 3.70-3.86 (m, 2H), 3.63-3.66 (m, 3H), 3.31 (s, 3H), 3.23 (s, 3H), 2.93-2.97 (m, 2H), 2.83-2.85 (m, 2H), 2.79 (s, 3H), 2.24 (s, 3H), 1.66 (m, 3H).

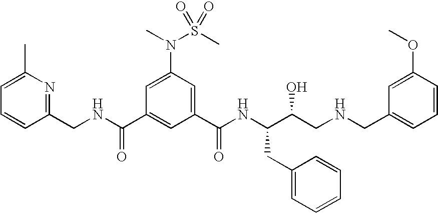

N1-((2S,3R)-3-hydroxy-4-(3-methoxybenzylamino)-1-phenylbutan-2-yl)-5-(N-methylmethylsulfonamido)-N3-((6-methylpyridin-2-yl)methyl)isophthalamide: ¹H NMR (300 MHz, CDCl₃) δ 8.14 (m, 2H), 8.02 (m, 1H), 7.87-7.81 (m, 2H), 7.62-7.60 (m, 1H), 7.30-7.08 (m, 9H), 6.93-6.90 (m, 2H), 6.82-6.79 (m, 1H), 4.70 (d, 2H), 4.45-4.41 (m, 1H), 3.91-3.72 (m, 6H), 3.33 (s, 3H), 3.02-2.73 (m, 8H), 2.55 (s, 3H).

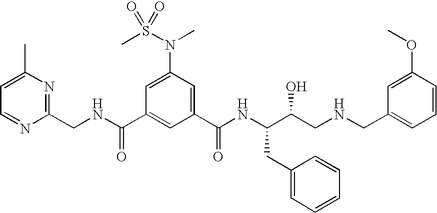

N1-((2S,3R)-3-hydroxy-4-(3-methoxybenzylamino)-1-phenylbutan-2-yl)-5-(N-methylmethyl sulfonamido)-N3-((4-methylpyrimidin-2-yl)methyl)isophthalamide: ¹H NMR (300 MHz, CDCl₃): 2.48 (s, 3H), 2.74-2.88 (m, 7H), 3.25 (s, 3H), 3.65-3.82 (m, 6H), 4.28-4.41 (m, 1H), 4.73-4.75 (m, 2H), 6.74-6.84 (m, 3H), 7.01-7.02 (d, 1H, J=5.4 Hz), 7.11-7.22 (m, 5H), 7.78-7.79 (m, 1H), 7.96-7.97 (m, 2H), 8.04 (br s, 1H), 8.45-8.47 (d, 1H, J=5.4 Hz).

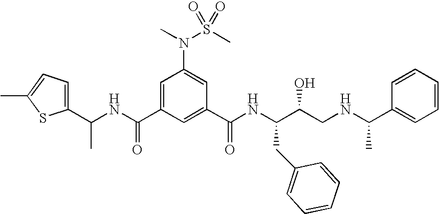

N1-((2S,3R)-3-hydroxy-1-phenyl-4-((S)-1-phenylethylamino)butan-2-yl)-5-(N-methylmethylsulfonamido)-N3-(1-(5-methylthiophen-2-yl)ethyl)isophthalamide. m/z: 663.

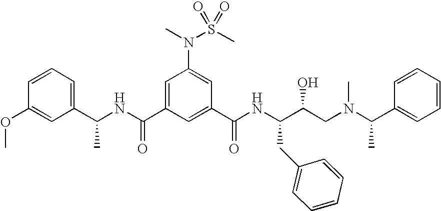

N1-((2S,3R)-3-hydroxy-4-(methyl((S)-1-phenylethyl)amino)-1-phenylbutan-2-yl)-N3-((R)-1-(3-methoxyphenyl)ethyl)-5-(N-methylmethyl sulfonamido)isophthalamide. m/z: 687.

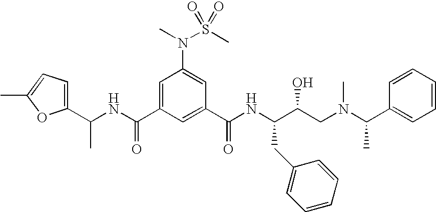

N1-((2S,3R)-3-hydroxy-4-(methyl((S)-1-phenylethyl)amino)-1-phenylbutan-2-yl)-N3-(1-(5-methylfuran-2-yl)ethyl)-5-(N-methylmethylsulfonamido)isophthalamide. m/z: 661.

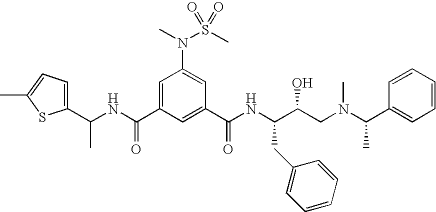

N1-((2S,3R)-3-hydroxy-4-(methyl((S)-1-phenylethyl)amino)-1-phenylbutan-2-yl)-5-(N-methylmethylsulfonamido)-N3-(1-(5-methylthiophen-2-yl)ethyl)isophthalamide. m/z: 676.

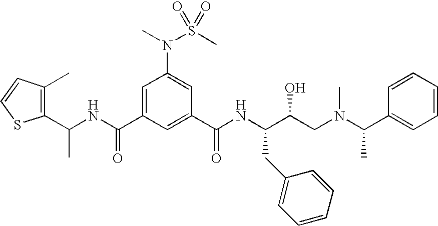

N1-((2S,3R)-3-hydroxy-4-(methyl((S)-1-phenylethyl)amino)-1-phenylbutan-2-yl)-5-(N-methylmethylsulfonamido)-N3-(1-(3-methylthiophen-2-yl)ethyl)isophthalamide. m/z: 677.

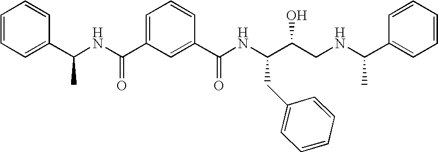

N1-((2S,3R)-3-hydroxy-1-phenyl-4-((S)-1-phenylethylamino)butan-2-yl)-N3-((S)-1-phenylethyl)isophthalamide. m/z: 536.

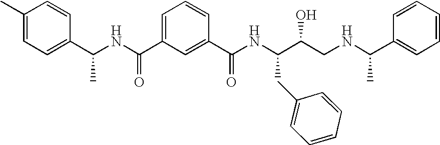

N1-((2S,3R)-3-hydroxy-1-phenyl-4-((S)-1-phenylethylamino)butan-2-yl)-N3-((R)-1-p-tolylethyl)isophthalamide. m/z: 550.

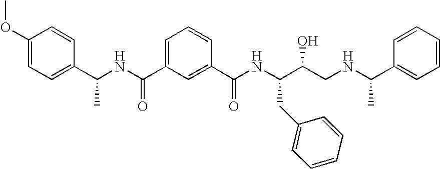

N1-((2S,3R)-3-hydroxy-1-phenyl-4-((S)-1-phenylethylamino)butan-2-yl)-N3-((R)-1-(4-methoxyphenyl)ethyl)isophthalamide. m/z: 566.

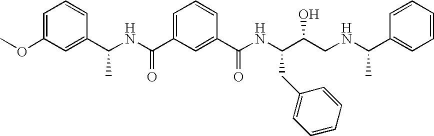

N1-((2S,3R)-3-hydroxy-1-phenyl-4-((S)-1-phenylethylamino)butan-2-yl)-N3-((R)-1-(3-methoxyphenyl)ethyl)isophthalamide. m/z: 566.

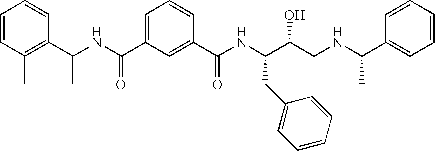

N1-((2S,3R)-3-hydroxy-1-phenyl-4-((S)-1-phenylethylamino)butan-2-yl)-N3-(1-o-tolylethyl)isophthalamide. m/z: 566.

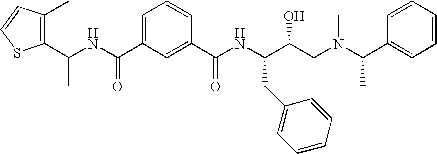

N1-((2S,3R)-3-hydroxy-4-(methyl((S)-1-phenylethyl)amino)-1-phenylbutan-2-yl)-N3-(1-(3-methylthiophen-2-yl)ethyl)isophthalamide. m/z: 570.

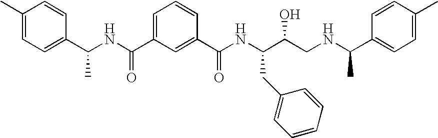

N1-((2S,3R)-3-hydroxy-1-phenyl-4-((R)-1-p-tolylethylamino)butan-2-yl)-N3-((R)-1-p-tolylethyl)isophthalamide. m/z: 564.

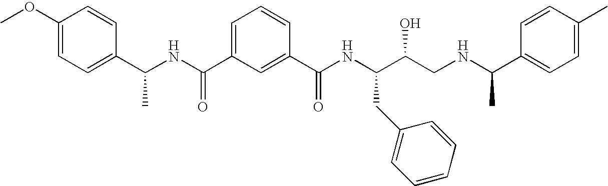

N1-((2S,3R)-3-hydroxy-1-phenyl-4-((R)-1-p-tolylethylamino)butan-2-yl)-N3-((R)-1-(4-methoxyphenyl)ethyl)isophthalamide. m/z: 580.

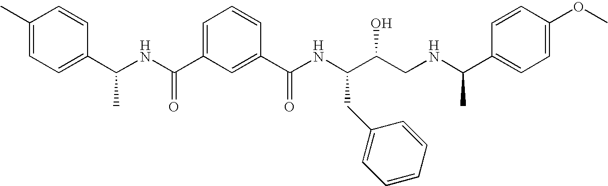

N1-((2S,3R)-3-hydroxy-4-((R)-1-(4-methoxyphenyl)ethylamino)-1-phenylbutan-2-yl)-N3-((R)-1-p-tolylethyl)isophthalamide. m/z: 580.

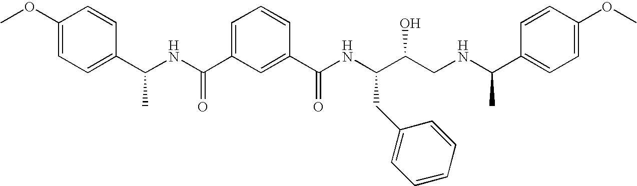

N1-((2S,3R)-3-hydroxy-4-((R)-1-(4-methoxyphenyl)ethylamino)-1-phenylbutan-2-yl)-N3-((R)-1-(4-methoxyphenyl)ethyl)isophthalamide. m/z: 596.

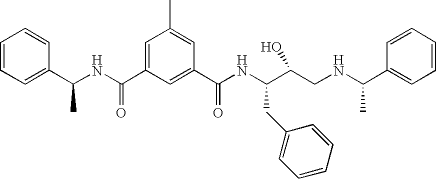

N1-((2S,3R)-3-hydroxy-1-phenyl-4-((S)-1-phenylethylamino)butan-2-yl)-5-methyl-N3-((S)-1-phenylethyl)isophthalamide. m/z: 550.

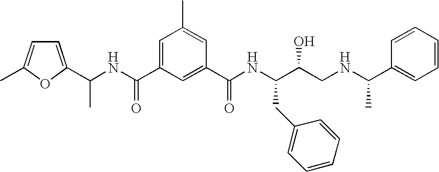

N1-((2S,3R)-3-hydroxy-1-phenyl-4-((S)-1-phenylethylamino)butan-2-yl)-5-methyl-N3-(1-(5-methylfuran-2-yl)ethyl)isophthalamide. m/z: 554.

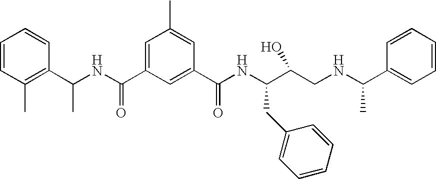

N1-((2S,3R)-3-hydroxy-1-phenyl-4-((S)-1-phenylethylamino)butan-2-yl)-5-methyl-N3-(1-o-tolylethyl)isophthalamide. m/z: 564.

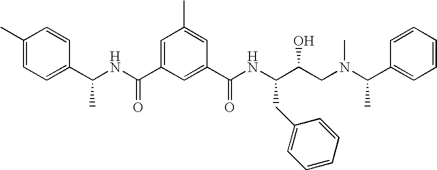

N1-((2S,3R)-3-hydroxy-4-(methyl((S)-1-phenylethyl)amino)-1-phenylbutan-2-yl)-5-methyl-N3-((R)-1-p-tolylethyl)isophthalamide. m/z: 578.

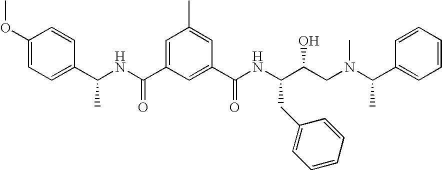

N1-((2S,3R)-3-hydroxy-4-(methyl((S)-1-phenylethyl)amino)-1-phenylbutan-2-yl)-N3-((R)-1-(4-methoxyphenyl)ethyl)-5-methylisophthalamide. m/z: 594.

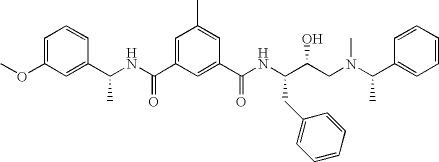

N1-((2S,3R)-3-hydroxy-4-(methyl((S)-1-phenylethyl)amino)-1-phenylbutan-2-yl)-N3-((R)-1-(3-methoxyphenyl)ethyl)-5-methylisophthalamide. m/z: 594.

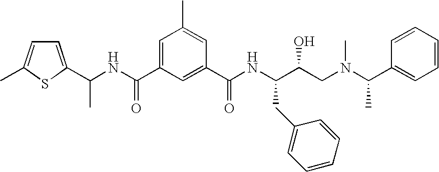

N1-((2S,3R)-3-hydroxy-4-(methyl((S)-1-phenylethyl)amino)-1-phenylbutan-2-yl)-5-methyl-N3-(1-(5-methylthiophen-2-yl)ethyl)isophthalamide. m/z: 584.

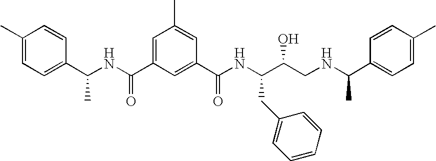

N1-((2S,3R)-3-hydroxy-1-phenyl-4-((R)-1-p-tolylethylamino)butan-2-yl)-5-methyl-N3-((R)-1-p-tolylethyl)isophthalamide. m/z: 578.

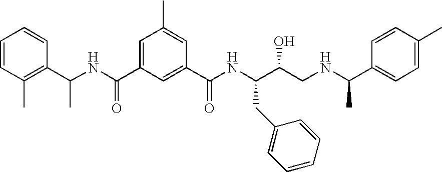

N1-((2S,3R)-3-hydroxy-1-phenyl-4-((R)-1-p-tolylethylamino)butan-2-yl)-5-methyl-N3-(1-o-tolylethyl)isophthalamide. m/z: 578.

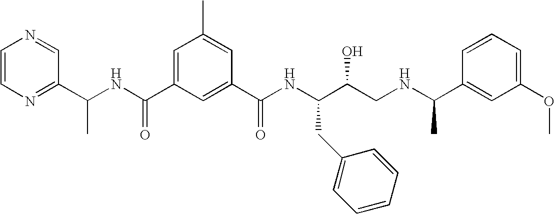

N1-((2S,3R)-3-hydroxy-4-((R)-1-(3-methoxyphenyl)ethylamino)-1-phenylbutan-2-yl)-5-methyl-N3-(1-(pyrazin-2-yl)ethyl)isophthalamide. m/z: 583.

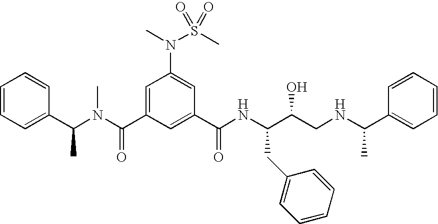

N1-((2S,3R)-3-hydroxy-1-phenyl-4-((S)-1-phenylethylamino)butan-2-yl)-N3-methyl-5-(N-methylmethylsulfonamido)-N3-((S)-1-phenylethyl)isophthalamide. m/z: 657.3.

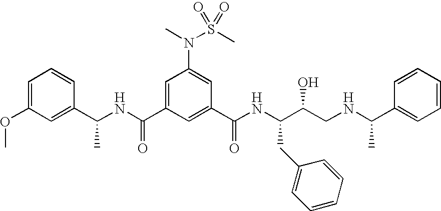

N1-((2S,3R)-3-hydroxy-1-phenyl-4-((S)-1-phenylethylamino)butan-2-yl)-N3-((R)-1-(3-methoxyphenyl)ethyl)-5-(N-methylmethylsulfonamido)isophthalamide. m/z: 673.3.

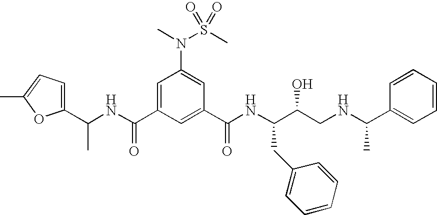

N1-((2S,3R)-3-hydroxy-1-phenyl-4-((S)-1-phenylethylamino)butan-2-yl)-N3-(1-(5-methylfuran-2-yl)ethyl)-5-(N-methylmethylsulfonamido)isophthalamide. m/z: 647.5.

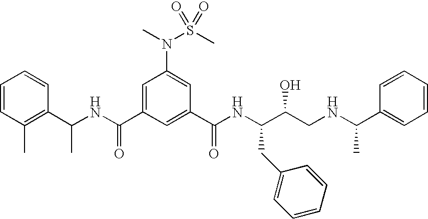

N1-((2S,3R)-3-hydroxy-1-phenyl-4-((S)-1-phenylethylamino)butan-2-yl)-5-(N-methylmethylsulfonamido)-N3-(1-o-tolylethyl)isophthalamide. m/z: 657.3.

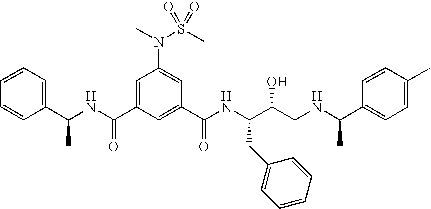

N1-((2S,3R)-3-hydroxy-1-phenyl-4-((R)-1-p-tolylethylamino)butan-2-yl)-5-(N-methylmethylsulfonamido)-N3-((S)-1-phenylethyl)isophthalamide. m/z: 657.3.

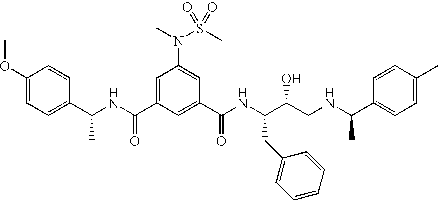

N1-((2S,3R)-3-hydroxy-1-phenyl-4-((R)-1-p-tolylethylamino)butan-2-yl)-N3-((R)-1-(4-methoxyphenyl)ethyl)-5-(N-methylmethylsulfonamido)isophthalamide. m/z: 687.5.

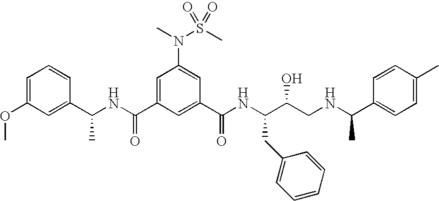

N1-((2S,3R)-3-hydroxy-1-phenyl-4-((R)-1-p-tolylethylamino)butan-2-yl)-N3-((R)-1-(3-methoxyphenyl)ethyl)-5-(N-methylmethylsulfonamido)isophthalamide. m/z: 687.3.

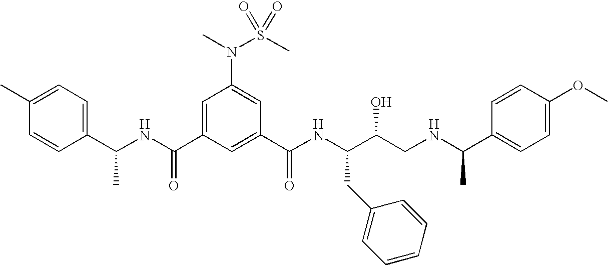

N1-((2S,3R)-3-hydroxy-4-((R)-1-(4-methoxyphenyl)ethylamino)-1-phenylbutan-2-yl)-5-(N-methylmethylsulfonamido)-N3-((R)-1-p-tolylethyl)isophthalamide. m/z: 687.5.

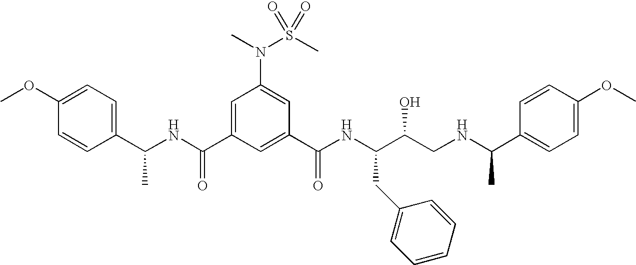

N1-((2S,3R)-3-hydroxy-4-((R)-1-(4-methoxyphenyl)ethylamino)-1-phenylbutan-2-yl)-N3-((R)-1-(4-methoxyphenyl)ethyl)-5-(N-methylmethylsulfonamido)isophthalamide. m/z: 703.5.

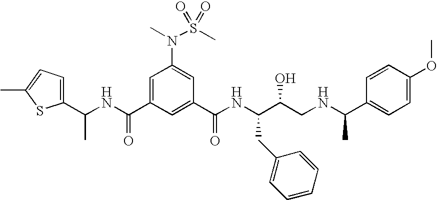

N1-((2S,3R)-3-hydroxy-4-((R)-1-(4-methoxyphenyl)ethylamino)-1-phenylbutan-2-yl)-5-(N-methylmethylsulfonamido)-N3-(1-(5-methylthiophen-2-yl)ethyl)isophthalamide. m/z: 693.3.

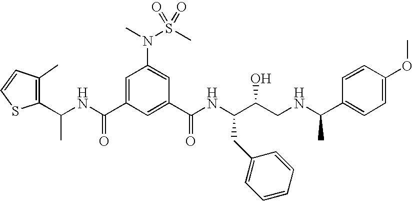

N1-((2S,3R)-3-hydroxy-4-((R)-1-(4-methoxyphenyl)ethylamino)-1-phenylbutan-2-yl)-5-(N-methylmethylsulfonamido)-N3-(1-(3-methylthiophen-2-yl)ethyl)isophthalamide. m/z: 693.5.

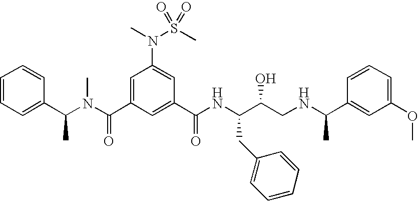

N1-((2S,3R)-3-hydroxy-4-((R)-1-(3-methoxyphenyl)ethylamino)-1-phenylbutan-2-yl)-N3-methyl-5-(N-methylmethylsulfonamido)-N3-((S)-1-phenylethyl)isophthalamide. m/z: 687.5.

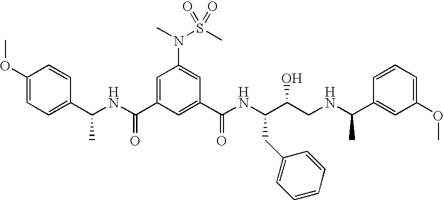

N1-((2S,3R)-3-hydroxy-4-((R)-1-(3-methoxyphenyl)ethylamino)-1-phenylbutan-2-yl)-N3-((R)-1-(4-methoxyphenyl)ethyl)-5-(N-methylmethylsulfonamido)isophthalamide. m/z: 703.5.

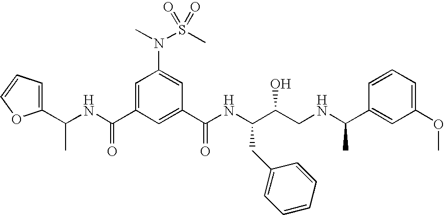

N1-(1-(furan-2-yl)ethyl)-N3-((2S,3R)-3-hydroxy-4-((R)-1-(3-methoxyphenyl)ethylamino)-1-phenylbutan-2-yl)-5-(N-methylmethylsulfonamido)isophthalamide. m/z: 663.5.

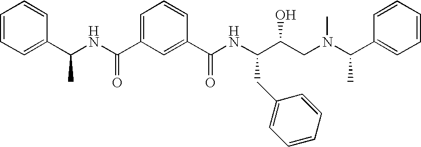

N1-((2S,3R)-3-hydroxy-4-(methyl((S)-1-phenylethyl)amino)-1-phenylbutan-2-yl)-N3-((S)-1-phenylethyl)isophthalamide. m/z: 550.3.

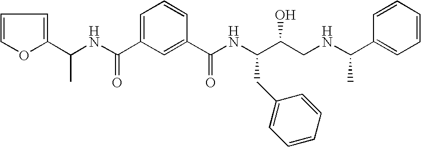

N1-(1-(furan-2-yl)ethyl)-N3-((2S,3R)-3-hydroxy-1-phenyl-4-((S)-1-phenylethylamino)butan-2-yl)isophthalamide. m/z: 526.2.

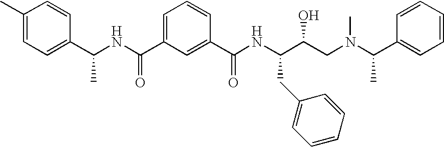

N1-((2S,3R)-3-hydroxy-4-(methyl((S)-1-phenylethyl)amino)-1-phenylbutan-2-yl)-N3-((R)-1-p-tolylethyl)isophthalamide. m/z: 564.3.

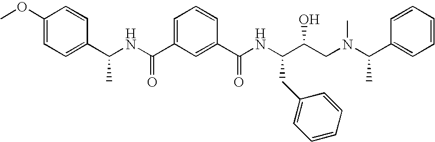

N1-((2S,3R)-3-hydroxy-4-(methyl((S)-1-phenylethyl)amino)-1-phenylbutan-2-yl)-N3-((R)-1-(4-methoxyphenyl)ethyl)isophthalamide. m/z: 580.3.

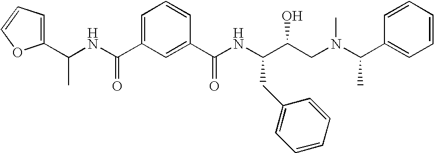

N1-(1-(furan-2-yl)ethyl)-N3-((2S,3R)-3-hydroxy-4-(methyl((S)-1-phenylethyl)amino)-1-phenylbutan-2-yl)isophthalamide. m/z: 540.3.

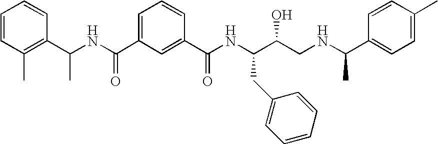

N1-((2S,3R)-3-hydroxy-1-phenyl-4-((R)-1-p-tolylethylamino)butan-2-yl)-N3-(1-o-tolylethyl)isophthalamide. m/z: 564.3.

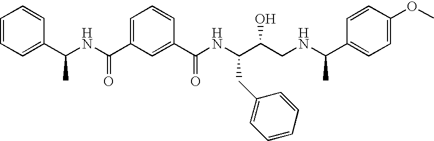

N1-((2S,3R)-3-hydroxy-4-((R)-1-(4-methoxyphenyl)ethylamino)-1-phenylbutan-2-yl)-N3-((S)-1-phenylethyl)isophthalamide. m/z: 566.3.

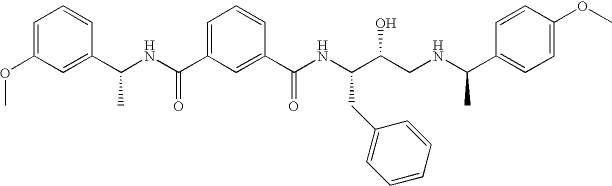

N1-((2S,3R)-3-hydroxy-4-((R)-1-(4-methoxyphenyl)ethylamino)-1-phenylbutan-2-yl)-N3-((R)-1-(3-methoxyphenyl)ethyl)isophthalamide. m/z: 596.3.

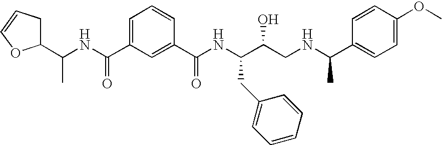

N1-(1-(2,3-dihydrofuran-2-yl)ethyl)-N3-((2S,3R)-3-hydroxy-4-((R)-1-(4-methoxyphenyl)ethylamino)-1-phenylbutan-2-yl)isophthalamide. m/z: 556.3.

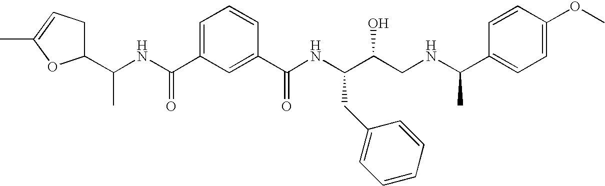

N1-((2S,3R)-3-hydroxy-4-((R)-1-(4-methoxyphenyl)ethylamino)-1-phenylbutan-2-yl)-N3-(1-(5-methyl-2,3-dihydrofuran-2-yl)ethyl)isophthalamide. m/z: 570.3.

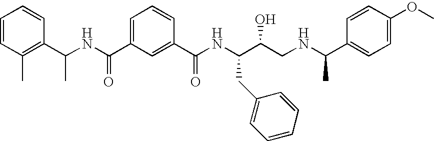

N1-((2S,3R)-3-hydroxy-4-((R)-1-(4-methoxyphenyl)ethylamino)-1-phenylbutan-2-yl)-N3-(1-o-tolylethyl)isophthalamide. m/z: 580.3.

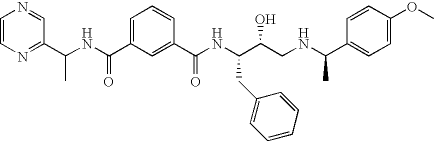

N1-((2S,3R)-3-hydroxy-4-((R)-1-(4-methoxyphenyl)ethylamino)-1-phenylbutan-2-yl)-N3-(1-(pyrazin-2-yl)ethyl)isophthalamide. m/z: 568.3.

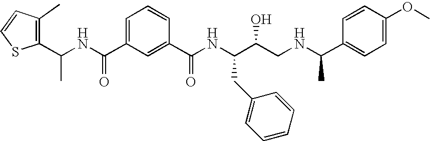

N1-((2S,3R)-3-hydroxy-4-((R)-1-(4-methoxyphenyl)ethylamino)-1-phenylbutan-2-yl)-N3-(1-(3-methylthiophen-2-yl)ethyl)isophthalamide. m/z: 586.3.

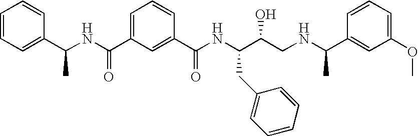

N1-((2S,3R)-3-hydroxy-4-((R)-1-(3-methoxyphenyl)ethylamino)-1-phenylbutan-2-yl)-N3-((S)-1-phenylethyl)isophthalamide. m/z: 566.3.

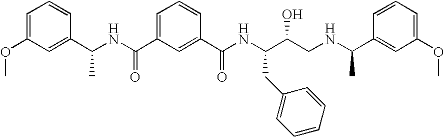

N1-((2S,3R)-3-hydroxy-4-((R)-1-(3-methoxyphenyl)ethylamino)-1-phenylbutan-2-yl)-N3-((R)-1-(3-methoxyphenyl)ethyl)isophthalamide. m/z: 596.3.

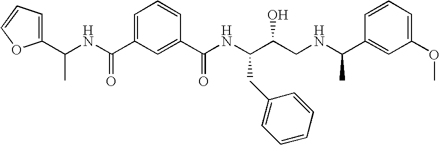

N1-(1-(furan-2-yl)ethyl)-N3-((2S,3R)-3-hydroxy-4-((R)-1-(3-methoxyphenyl)ethylamino)-1-phenylbutan-2-yl)isophthalamide. m/z: 556.3.

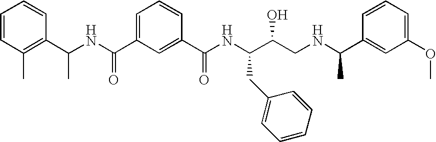

N1-((2S,3R)-3-hydroxy-4-((R)-1-(3-methoxyphenyl)ethylamino)-1-phenylbutan-2-yl)-N3-(1-o-tolylethyl)isophthalamide. m/z: 580.3.

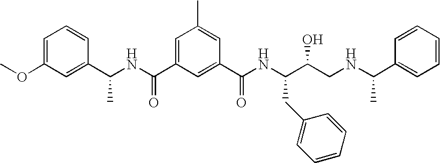

N1-((2S,3R)-3-hydroxy-1-phenyl-4-((S)-1-phenylethylamino)butan-2-yl)-N3-((R)-1-(3-methoxyphenyl)ethyl)-5-methylisophthalamide. m/z: 580.5.

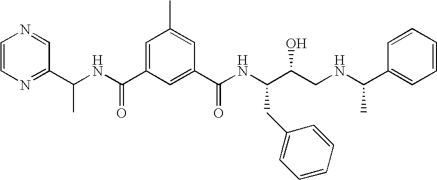

N1-((2S,3R)-3-hydroxy-1-phenyl-4-((S)-1-phenylethylamino)butan-2-yl)-5-methyl-N3-(1-(pyrazin-2-yl)ethyl)isophthalamide. m/z: 552.3.

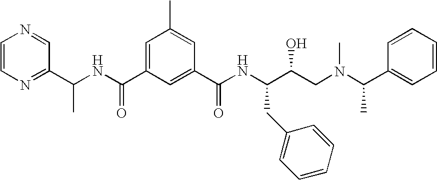

N1-((2S,3R)-3-hydroxy-4-(methyl((S)-1-phenylethyl)amino)-1-phenylbutan-2-yl)-5-methyl-N3-(1-(pyrazin-2-yl)ethyl)isophthalamide. m/z: 566.3.

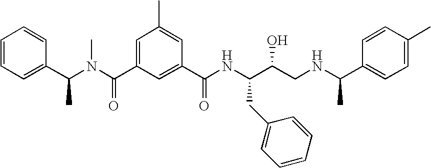

N1-((2S,3R)-3-hydroxy-1-phenyl-4-((R)-1-p-tolylethylamino)butan-2-yl)-N3,5-dimethyl-N3-((S)-1-phenylethyl)isophthalamide. m/z: 578.5.

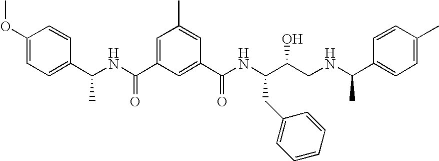

N1-((2S,3R)-3-hydroxy-1-phenyl-4-((R)-1-p-tolylethylamino)butan-2-yl)-N3-((R)-1-(4-methoxyphenyl)ethyl)-5-methylisophthalamide. m/z: 594.5.

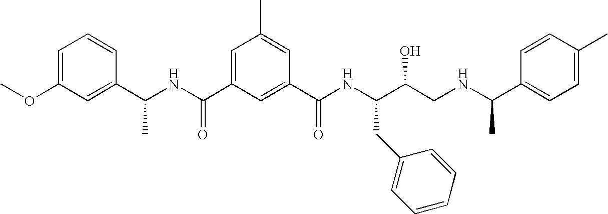

N1-((2S,3R)-3-hydroxy-1-phenyl-4-((R)-1-p-tolylethylamino)butan-2-yl)-N3-((R)-1-(3-methoxyphenyl)ethyl)-5-methylisophthalamide. m/z: 594.5.

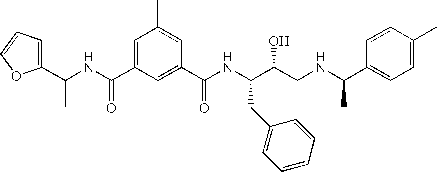

N1-(1-(furan-2-yl)ethyl)-N3-((2S,3R)-3-hydroxy-1-phenyl-4-((R)-1-p-tolylethylamino)butan-2-yl)-5-methylisophthalamide. m/z: 554.3.

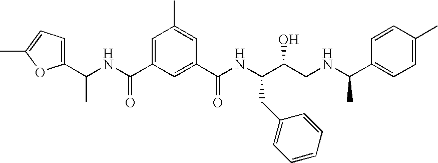

N1-((2S,3R)-3-hydroxy-1-phenyl-4-((R)-1-p-tolylethylamino)butan-2-yl)-5-methyl-N3-(1-(5-methylfuran-2-yl)ethyl)isophthalamide. m/z: 568.5.

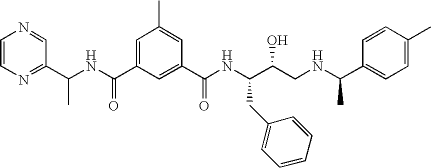

N1-((2S,3R)-3-hydroxy-1-phenyl-4-((R)-1-p-tolylethylamino)butan-2-yl)-5-methyl-N3-(1-(pyrazin-2-yl)ethyl)isophthalamide. m/z: 566.5.

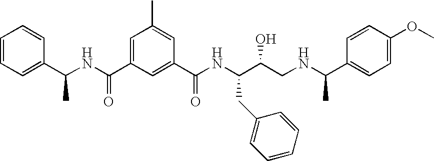

N1-((2S,3R)-3-hydroxy-4-((R)-1-(4-methoxyphenyl)ethylamino)-1-phenylbutan-2-yl)-5-methyl-N3-((S)-1-phenylethyl)isophthalamide. m/z: 580.3.

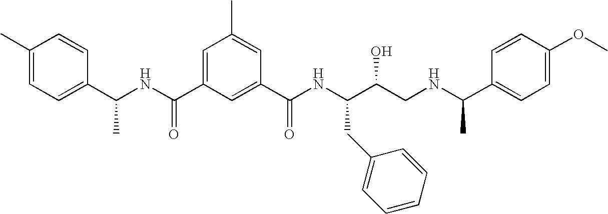

N1-((2S,3R)-3-hydroxy-4-((R)-1-(4-methoxyphenyl)ethylamino)-1-phenylbutan-2-yl)-5-methyl-N3-((R)-1-p-tolylethyl)isophthalamide. m/z: 594.3.

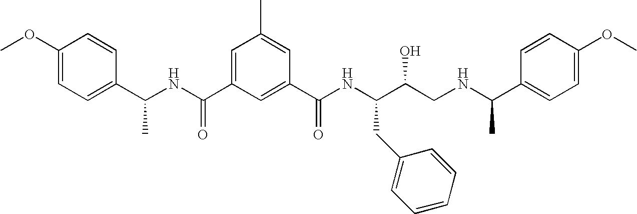

N1-((2S,3R)-3-hydroxy-4-((R)-1-(4-methoxyphenyl)ethylamino)-1-phenylbutan-2-yl)-N3-((R)-1-(4-methoxyphenyl)ethyl)-5-methylisophthalamide. m/z: 610.3.

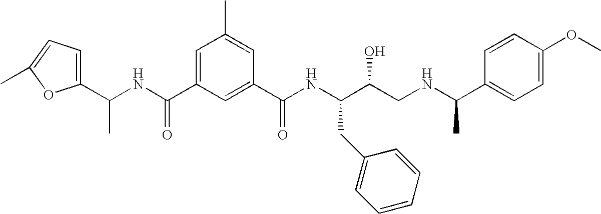

N1-((2S,3R)-3-hydroxy-4-((R)-1-(4-methoxyphenyl)ethylamino)-1-phenylbutan-2-yl)-5-methyl-N3-(1-(5-methylfuran-2-yl)ethyl)isophthalamide. m/z: 584.3.

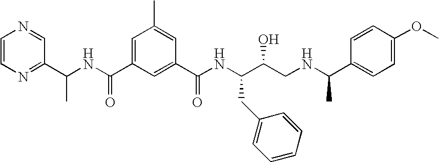

N1-((2S,3R)-3-hydroxy-4-((R)-1-(4-methoxyphenyl)ethylamino)-1-phenylbutan-2-yl)-5-methyl-N3-(1-(pyrazin-2-yl)ethyl)isophthalamide. m/z: 582.3.

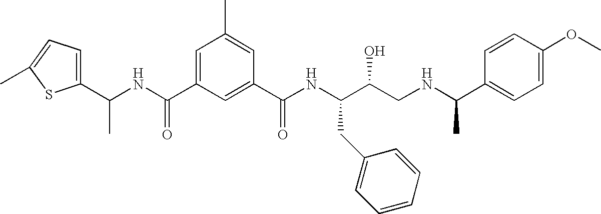

N1-((2S,3R)-3-hydroxy-4-((R)-1-(4-methoxyphenyl)ethylamino)-1-phenylbutan-2-yl)-5-methyl-N3-(1-(5-methylthiophen-2-yl)ethyl)isophthalamide. m/z: 600.3.

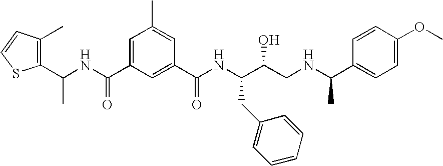

N1-((2S,3R)-3-hydroxy-4-((R)-1-(4-methoxyphenyl)ethylamino)-1-phenylbutan-2-yl)-5-methyl-N3-(1-(3-methylthiophen-2-yl)ethyl)isophthalamide. m/z: 600.3.

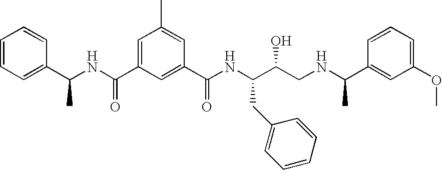

N1-((2S,3R)-3-hydroxy-4-((R)-1-(3-methoxyphenyl)ethylamino)-1-phenylbutan-2-yl)-5-methyl-N3-((S)-1-phenylethyl)isophthalamide. m/z: 580.3.

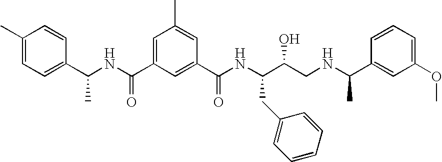

N1-((2S,3R)-3-hydroxy-4-((R)-1-(3-methoxyphenyl)ethylamino)-1-phenylbutan-2-yl)-5-methyl-N3-((R)-1-p-tolylethyl)isophthalamide. m/z: 594.5.

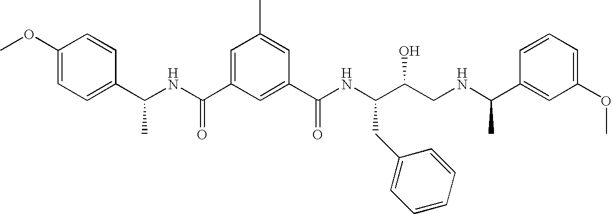

N1-((2S,3R)-3-hydroxy-4-((R)-1-(3-methoxyphenyl)ethylamino)-1-phenylbutan-2-yl)-N3-((R)-1-(4-methoxyphenyl)ethyl)-5-methylisophthalamide. m/z: 610.3.

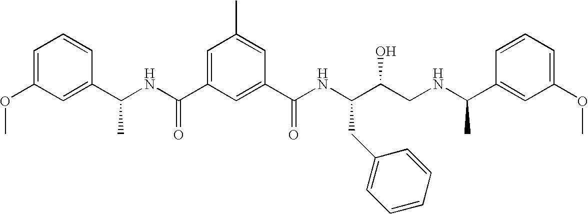

N1-((2S,3R)-3-hydroxy-4-((R)-1-(3-methoxyphenyl)ethylamino)-1-phenylbutan-2-yl)-N3-((R)-1-(3-methoxyphenyl)ethyl)-5-methylisophthalamide. m/z: 610.3.

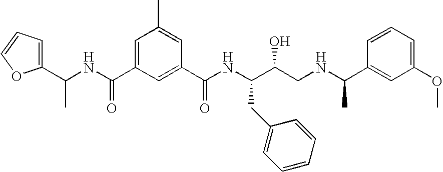

N1-(1-(furan-2-yl)ethyl)-N3-((2S,3R)-3-hydroxy-4-((R)-1-(3-methoxyphenyl)ethylamino)-1-phenylbutan-2-yl)-5-methylisophthalamide. m/z: 570.5.

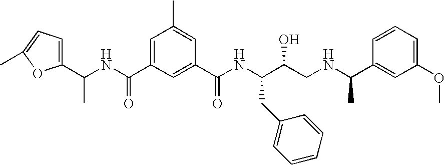

N1-((2S,3R)-3-hydroxy-4-((R)-1-(3-methoxyphenyl)ethylamino)-1-phenylbutan-2-yl)-5-methyl-N3-(1-(5-methylfuran-2-yl)ethyl)isophthalamide. m/z: 584.3.

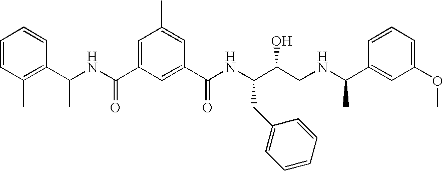

N1-((2S,3R)-3-hydroxy-4-((R)-1-(3-methoxyphenyl)ethylamino)-1-phenylbutan-2-yl)-5-methyl-N3-(1-o-tolylethyl)isophthalamide. m/z: 594.3.

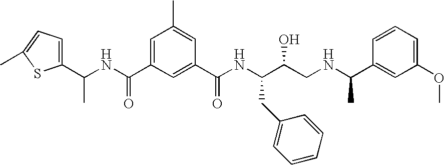

N1-((2S,3R)-3-hydroxy-4-((R)-1-(3-methoxyphenyl)ethylamino)-1-phenylbutan-2-yl)-5-methyl-N3-(1-(5-methylthiophen-2-yl)ethyl)isophthalamide. m/z: 600.5.

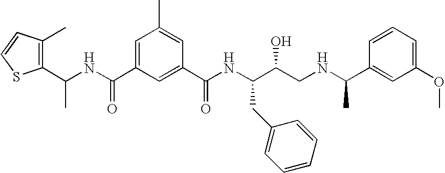

N1-((2S,3R)-3-hydroxy-4-((R)-1-(3-methoxyphenyl)ethylamino)-1-phenylbutan-2-yl)-5-methyl-N3-(1-(3-methylthiophen-2-yl)ethyl)isophthalamide. m/z: 600.3.

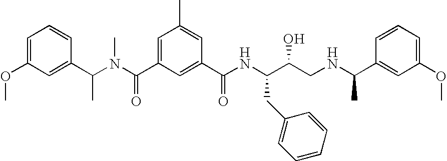

N1-((2S,3R)-3-hydroxy-4-((R)-1-(3-methoxyphenyl)ethylamino)-1-phenylbutan-2-yl)-N3-(1-(3-methoxyphenyl)ethyl)-N3,5-dimethylisophthalamide. m/z: 624.5.

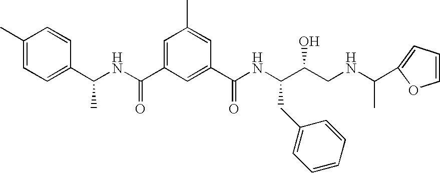

N1-((2S,3R)-4-(1-(furan-2-yl)ethylamino)-3-hydroxy-1-phenylbutan-2-yl)-5-methyl-N3-((R)-1-p-tolylethyl)isophthalamide. m/z: 554.5.

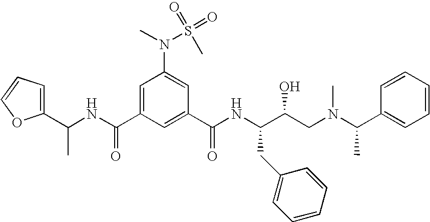

N1-(1-(furan-2-yl)ethyl)-N3-((2S,3R)-3-hydroxy-4-(methyl((S)-1-phenylethyl)amino)-1-phenylbutan-2-yl)-5-(N-methylmethylsulfonamido)isophthalamide. m/z: 647.3.

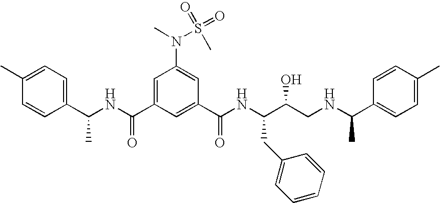

N1-((2S,3R)-3-hydroxy-1-phenyl-4-((R)-1-p-tolylethylamino)butan-2-yl)-5-(N-methylmethylsulfonamido)-N3-((R)-1-p-tolylethyl)isophthalamide. m/z: 671.3.

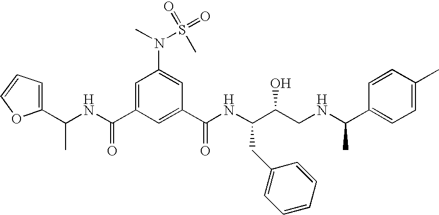

N1-(1-(furan-2-yl)ethyl)-N3-((2S,3R)-3-hydroxy-1-phenyl-4-((R)-1-p-tolylethylamino)butan-2-yl)-5-(N-methylmethylsulfonamido)isophthalamide. m/z: 647.3.

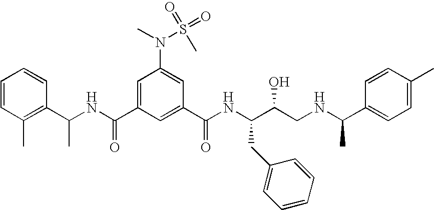

N1-((2S,3R)-3-hydroxy-1-phenyl-4-((R)-1-p-tolylethylamino)butan-2-yl)-5-(N-methylmethylsulfonamido)-N3-(1-o-tolylethyl)isophthalamide. m/z: 671.3.

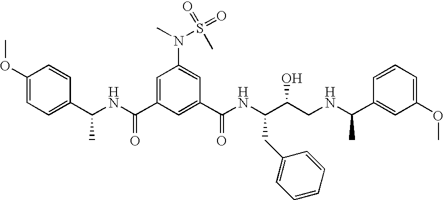

N1-((2S,3R)-3-hydroxy-4-((R)-1-(3-methoxyphenyl)ethylamino)-1-phenylbutan-2-yl)-N3-((R)-1-(4-methoxyphenyl)ethyl)-5-(N-methylmethylsulfonamido)isophthalamide. m/z: 703.3.

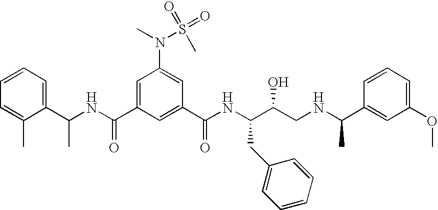

N1-((2S,3R)-3-hydroxy-4-((R)-1-(3-methoxyphenyl)ethylamino)-1-phenylbutan-2-yl)-5-(N-methylmethylsulfonamido)-N3-(1-o-tolylethyl)isophthalamide. m/z: 687.3.

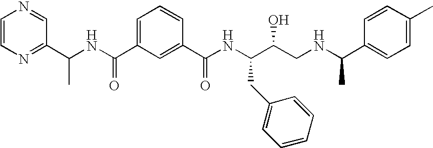

N1-((2S,3R)-3-hydroxy-1-phenyl-4-((R)-1-p-tolylethylamino)butan-2-yl)-N3-(1-(pyrazin-2-yl)ethyl)isophthalamide. m/z: 552.3.

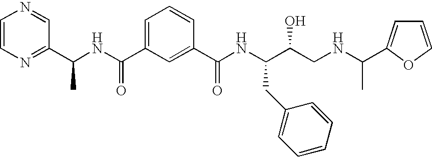

N1-((2S,3R)-4-(1-(furan-2-yl)ethylamino)-3-hydroxy-1-phenylbutan-2-yl)-N3-((S)-1-phenylethyl)isophthalamide. m/z: 526.2.

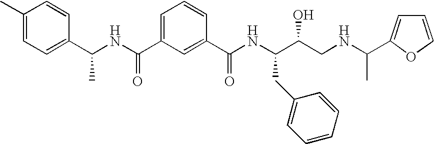

N1-((2S,3R)-4-(1-(furan-2-yl)ethylamino)-3-hydroxy-1-phenylbutan-2-yl)-N3-((R)-1-p-tolylethyl)isophthalamide. m/z: 540.3.

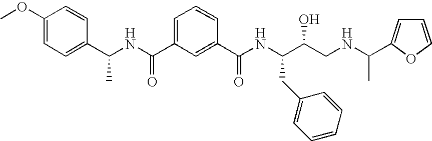

N1-((2S,3R)-4-(1-(furan-2-yl)ethylamino)-3-hydroxy-1-phenylbutan-2-yl)-N3-((R)-1-(4-methoxyphenyl)ethyl)isophthalamide. m/z: 556.3.

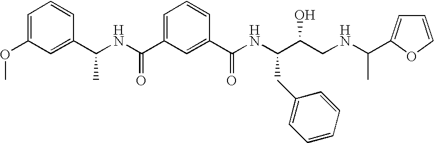

N1-((2S,3R)-4-(1-(furan-2-yl)ethylamino)-3-hydroxy-1-phenylbutan-2-yl)-N3-((R)-1-(3-methoxyphenyl)ethyl)isophthalamide. m/z: 556.3.

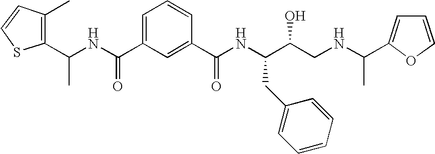

N1-((2S,3R)-4-(1-(furan-2-yl)ethylamino)-3-hydroxy-1-phenylbutan-2-yl)-N3-(1-(3-methylthiophen-2-yl)ethyl)isophthalamide. m/z: 546.3.

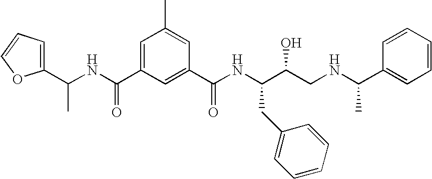

N1-(1-(furan-2-yl)ethyl)-N3-((2S,3R)-3-hydroxy-1-phenyl-4-((S)-1-phenylethylamino)butan-2-yl)-5-methylisophthalamide. m/z: 540.3.

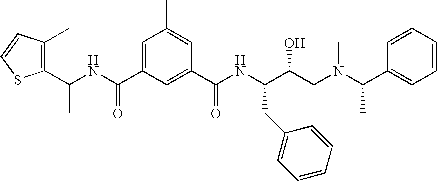

N1-((2S,3R)-3-hydroxy-4-(methyl((S)-1-phenylethyl)amino)-1-phenylbutan-2-yl)-5-methyl-N3-(1-(3-methylthiophen-2-yl)ethyl)isophthalamide. m/z: 584.3.

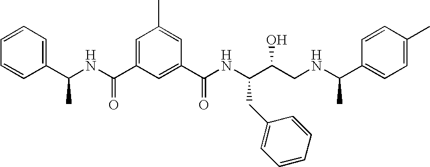

N1-((2S,3R)-3-hydroxy-1-phenyl-4-((R)-1-p-tolylethylamino)butan-2-yl)-5-methyl-N3-((S)-1-phenylethyl)isophthalamide. m/z: 564.3.

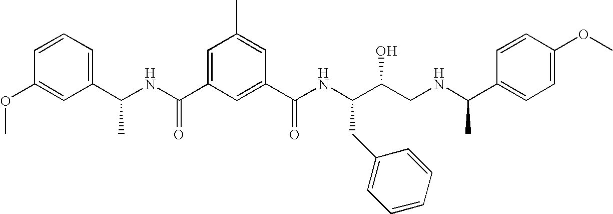

N1-((2S,3R)-3-hydroxy-4-((R)-1-(4-methoxyphenyl)ethylamino)-1-phenylbutan-2-yl)-N3-((R)-1-(3-methoxyphenyl)ethyl)-5-methylisophthalamide. m/z: 610.3.

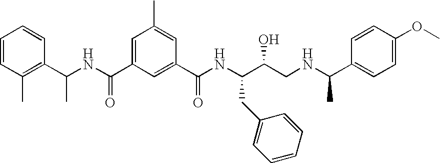

N1-((2S,3R)-3-hydroxy-4-((R)-1-(4-methoxyphenyl)ethylamino)-1-phenylbutan-2-yl)-5-methyl-N3-(1-o-tolylethyl)isophthalamide. m/z: 594.3.

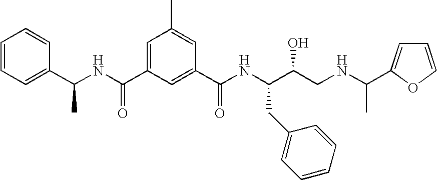

N1-((2S,3R)-4-(1-(furan-2-yl)ethylamino)-3-hydroxy-1-phenylbutan-2-yl)-5-methyl-N3-((S)-1-phenylethyl)isophthalamide. m/z: 540.3.

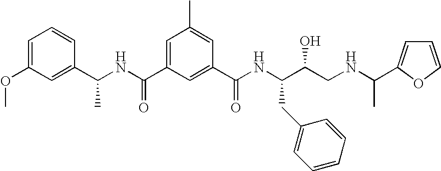

N1-((2S,3R)-4-(1-(furan-2-yl)ethylamino)-3-hydroxy-1-phenylbutan-2-yl)-N3-((R)-1-(3-methoxyphenyl)ethyl)-5-methylisophthalamide. m/z: 570.3.

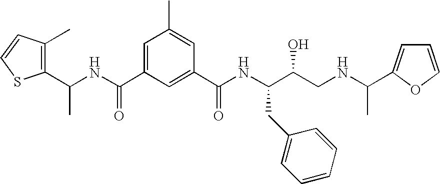

N1-((2S,3R)-4-(1-(furan-2-yl)ethylamino)-3-hydroxy-1-phenylbutan-2-yl)-5-methyl-N3-(1-(3-methylthiophen-2-yl)ethyl)isophthalamide. m/z: 560.3.

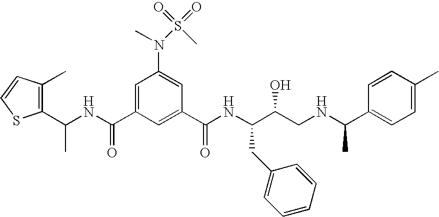

N1-((2S,3R)-3-hydroxy-1-phenyl-4-((R)-1-p-tolylethylamino)butan-2-yl)-5-(N-methylmethylsulfonamido)-N3-(1-(3-methylthiophen-2-yl)ethyl)isophthalamide. m/z: 677.3.

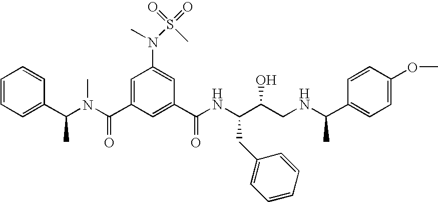

N1-((2S,3R)-3-hydroxy-4-((R)-1-(4-methoxyphenyl)ethylamino)-1-phenylbutan-2-yl)-N3-methyl-5-(N-methylmethylsulfonamido)-N3-((S)-1-phenylethyl)isophthalamide. m/z: 687.3.

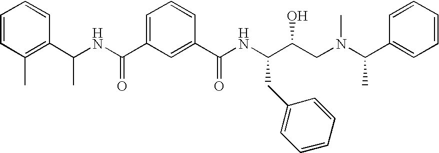

N1-((2S,3R)-3-hydroxy-4-(methyl((S)-1-phenylethyl)amino)-1-phenylbutan-2-yl)-N3-(1-o-tolylethyl)isophthalamide. m/z: 564.3.

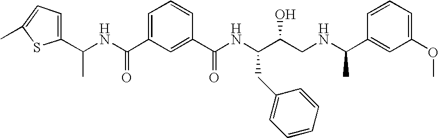

N1-((2S,3R)-3-hydroxy-4-((R)-1-(3-methoxyphenyl)ethylamino)-1-phenylbutan-2-yl)-N3-(1-(5-methylthiophen-2-yl)ethyl)isophthalamide. m/z: 586.3.

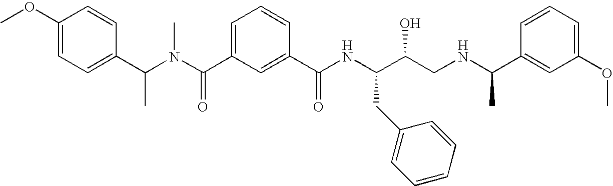

N1-((2S,3R)-3-hydroxy-4-((R)-1-(3-methoxyphenyl)ethylamino)-1-phenylbutan-2-yl)-N3-(1-(4-methoxyphenyl)ethyl)-N3-methylisophthalamide. m/z: 610.3.

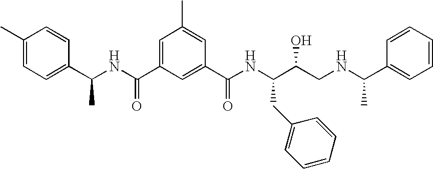

N1-((2S,3R)-3-hydroxy-1-phenyl-4-((S)-1-phenylethylamino)butan-2-yl)-5-methyl-N3-((S)-1-p-tolylethyl)isophthalamide. m/z: 564.3.

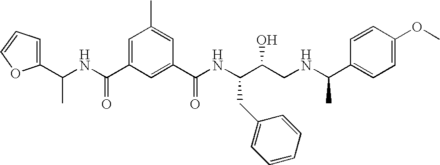

N1-(1-(furan-2-yl)ethyl)-N3-((2S,3R)-3-hydroxy-4-((R)-1-(4-methoxyphenyl)ethylamino)-1-phenylbutan-2-yl)-5-methylisophthalamide. m/z: 570.3.

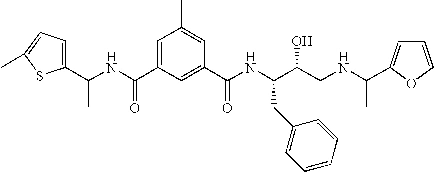

N1-((2S,3R)-4-(1-(furan-2-yl)ethylamino)-3-hydroxy-1-phenylbutan-2-yl)-5-methyl-N3-(1-(5-methylthiophen-2-yl)ethyl)isophthalamide. m/z: 560.3.

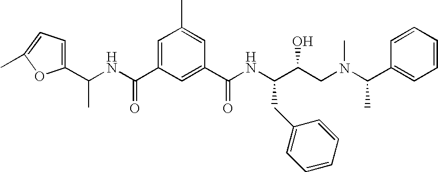

N1-((2S,3R)-3-hydroxy-4-(methyl((S)-1-phenylethyl)amino)-1-phenylbutan-2-yl)-5-methyl-N3-(1-(5-methylfuran-2-yl)ethyl)isophthalamide. m/z: 568.3.

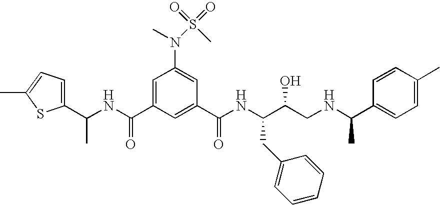

N1-((2S,3R)-3-hydroxy-1-phenyl-4-((R)-1-p-tolylethylamino)butan-2-yl)-5-(N-methylmethylsulfonamido)-N3-(1-(5-methylthiophen-2-yl)ethyl)isophthalamide. m/z: 677.3.

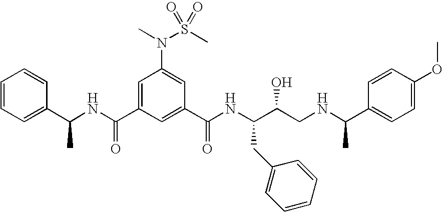

N1-((2S,3R)-3-hydroxy-4-((R)-1-(4-methoxyphenyl)ethylamino)-1-phenylbutan-2-yl)-5-(N-methylmethylsulfonamido)-N3-((S)-1-phenylethyl)isophthalamide. m/z: 673.3.

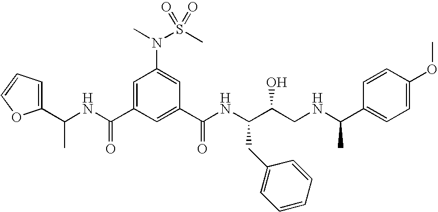

N1-(1-(furan-2-yl)ethyl)-N3-((2S,3R)-3-hydroxy-4-((R)-1-(4-methoxyphenyl)ethylamino)-1-phenylbutan-2-yl)-5-(N-methylmethylsulfonamido)isophthalamide. m/z: 663.3.

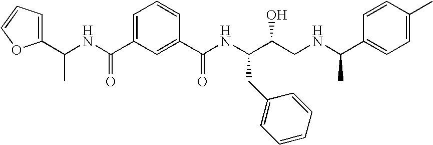

N1-(1-(furan-2-yl)ethyl)-N3-((2S,3R)-3-hydroxy-1-phenyl-4-((R)-1-p-tolylethylamino)butan-2-yl)isophthalamide. m/z: 540.3.

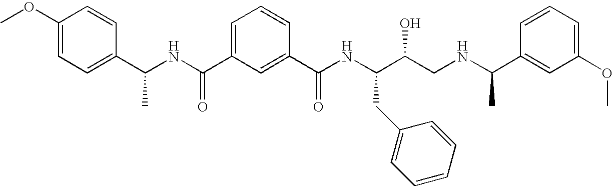

N1-((2S,3R)-3-hydroxy-4-((R)-1-(3-methoxyphenyl)ethylamino)-1-phenylbutan-2-yl)-N3-((R)-1-(4-methoxyphenyl)ethyl)isophthalamide. m/z: 596.3.

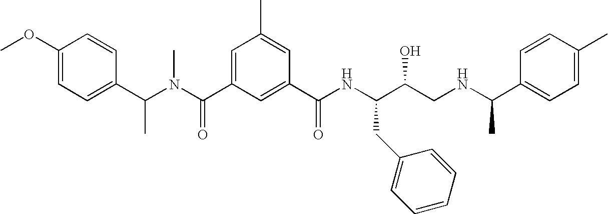

N1-((2S,3R)-3-hydroxy-1-phenyl-4-((R)-1-p-tolylethylamino)butan-2-yl)-N3-(1-(4-methoxyphenyl)ethyl)-N3,5-dimethylisophthalamide. m/z: 608.3.

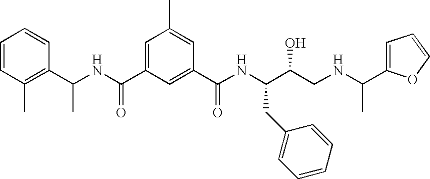

N1-((2S,3R)-4-(1-(furan-2-yl)ethylamino)-3-hydroxy-1-phenylbutan-2-yl)-5-methyl-N3-(1-o-tolylethyl)isophthalamide. m/z: 554.5.

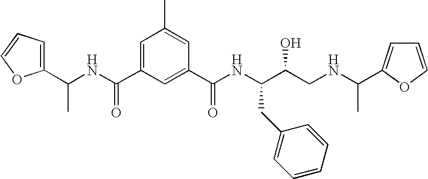

N1-(1-(furan-2-yl)ethyl)-N3-((2S,3R)-4-(1-(furan-2-yl)ethylamino)-3-hydroxy-1-phenylbutan-2-yl)-5-methylisophthalamide. m/z: 530.3.

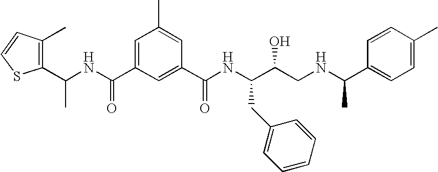

N1-((2S,3R)-3-hydroxy-1-phenyl-4-((R)-1-p-tolylethylamino)butan-2-yl)-5-methyl-N3-(1-(3-methylthiophen-2-yl)ethyl)isophthalamide. m/z: 584.3.

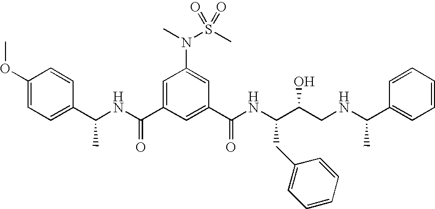

N1-((2S,3R)-3-hydroxy-1-phenyl-4-((S)-1-phenylethylamino)butan-2-yl)-N3-((R)-1-(4-methoxyphenyl)ethyl)-5-(N-methylmethylsulfonamido)isophthalamide. m/z: 673.3.

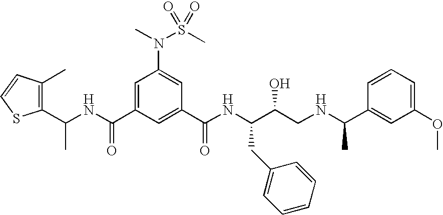

N1-((2S,3R)-3-hydroxy-4-((R)-1-(3-methoxyphenyl)ethylamino)-1-phenylbutan-2-yl)-5-(N-methylmethylsulfonamido)-N3-(1-(3-methylthiophen-2-yl)ethyl)isophthalamide. m/z: 693.3.

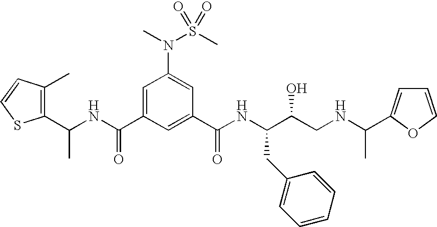

N1-((2S,3R)-4-(1-(furan-2-yl)ethylamino)-3-hydroxy-1-phenylbutan-2-yl)-5-(N-methylmethylsulfonamido)-N3-(1-(3-methylthiophen-2-yl)ethyl)isophthalamide. m/z: 653.3.

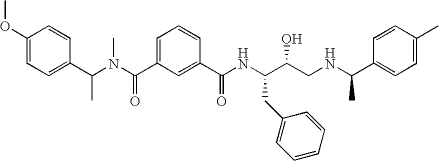

N1-((2S,3R)-3-hydroxy-1-phenyl-4-((R)-1-p-tolylethylamino)butan-2-yl)-N3-(1-(4-methoxyphenyl)ethyl)-N3-methylisophthalamide. m/z: 594.

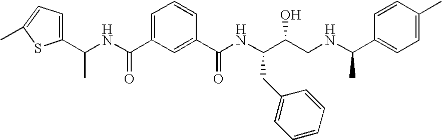

N1-((2S,3R)-3-hydroxy-1-phenyl-4-((R)-1-p-tolylethylamino)butan-2-yl)-N3-(1-(5-methylthiophen-2-yl)ethyl)isophthalamide. m/z: 570.3.

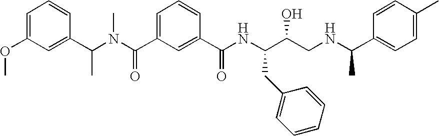

N1-((2S,3R)-3-hydroxy-1-phenyl-4-((R)-1-p-tolylethylamino)butan-2-yl)-N3-(1-(3-methoxyphenyl)ethyl)-N3-methylisophthalamide. m/z: 608.3.

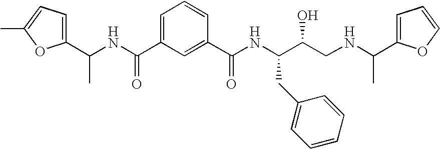

N1-((2S,3R)-4-(1-(furan-2-yl)ethylamino)-3-hydroxy-1-phenylbutan-2-yl)-N3-(1-(5-methylfuran-2-yl)ethyl)isophthalamide. m/z: 530.3.

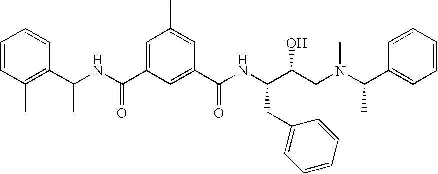

N1-((2S,3R)-3-hydroxy-4-(methyl((S)-1-phenylethyl)amino)-1-phenylbutan-2-yl)-5-methyl-N3-(1-o-tolylethyl)isophthalamide. m/z: 578.5.

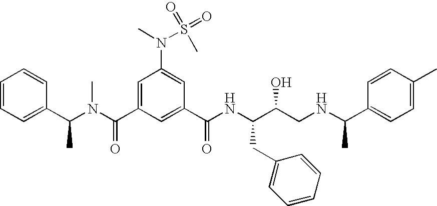

N1-((2S,3R)-3-hydroxy-1-phenyl-4-((R)-1-p-tolylethylamino)butan-2-yl)-N3-methyl-5-(N-methylmethylsulfonamido)-N3-((S)-1-phenylethyl)isophthalamide. m/z: 671.3.

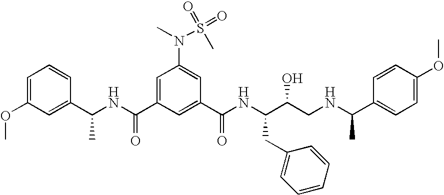

N1-((2S,3R)-3-hydroxy-4-((R)-1-(4-methoxyphenyl)ethylamino)-1-phenylbutan-2-yl)-N3-((R)-1-(3-methoxyphenyl)ethyl)-5-(N-methylmethylsulfonamido)isophthalamide. m/z: 703.3.

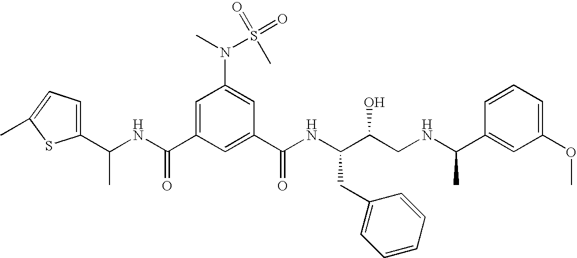

N1-((2S,3R)-3-hydroxy-4-((R)-1-(3-methoxyphenyl)ethylamino)-1-phenylbutan-2-yl)-5-(N-methylmethylsulfonamido)-N3-(1-(5-methylthiophen-2-yl)ethyl)isophthalamide. m/z: 693.3.

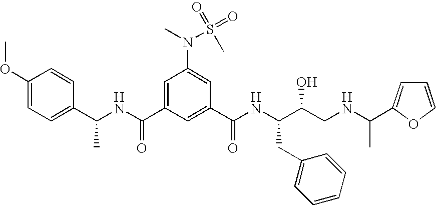

N1-((2S,3R)-4-(1-(furan-2-yl)ethylamino)-3-hydroxy-1-phenylbutan-2-yl)-N3-((R)-1-(4-methoxyphenyl)ethyl)-5-(N-methylmethylsulfonamido)isophthalamide. m/z: 663.3.

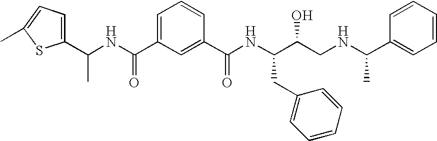

N1-((2S,3R)-3-hydroxy-1-phenyl-4-((S)-1-phenylethylamino)butan-2-yl)-N3-(1-(5-methylthiophen-2-yl)ethyl)isophthalamide. m/z: 556.3.

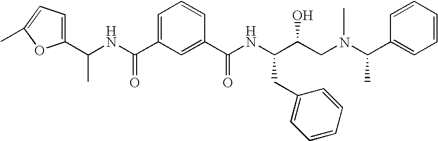

N1-((2S,3R)-3-hydroxy-4-(methyl((S)-1-phenylethyl)amino)-1-phenylbutan-2-yl)-N3-(1-(5-methylfuran-2-yl)ethyl)isophthalamide. m/z: 554.3.

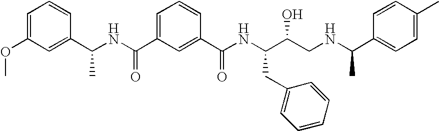

N1-((2S,3R)-3-hydroxy-1-phenyl-4-((R)-1-p-tolylethylamino)butan-2-yl)-N3-((R)-1-(3-methoxyphenyl)ethyl)isophthalamide. m/z: 580.3.

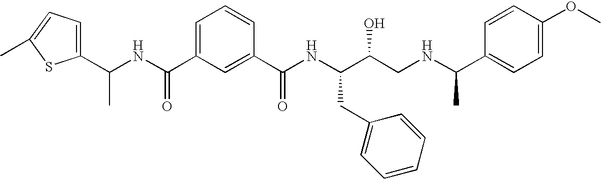

N1-((2S,3R)-3-hydroxy-4-((R)-1-(4-methoxyphenyl)ethylamino)-1-phenylbutan-2-yl)-N3-(1-(5-methylthiophen-2-yl)ethyl)isophthalamide. m/z: 586.3.

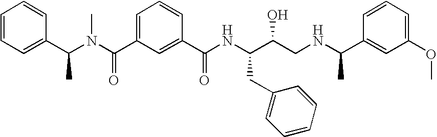

N1-((2S,3R)-3-hydroxy-4-((R)-1-(3-methoxyphenyl)ethylamino)-1-phenylbutan-2-yl)-N3-methyl-N3-((S)-1-phenylethyl)isophthalamide. m/z: 580.3.

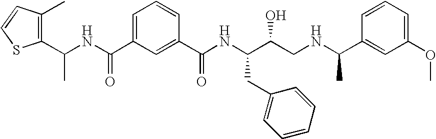

N1-((2S,3R)-3-hydroxy-4-((R)-1-(3-methoxyphenyl)ethylamino)-1-phenylbutan-2-yl)-N3-(1-(3-methylthiophen-2-yl)ethyl)isophthalamide. m/z: 586.3.

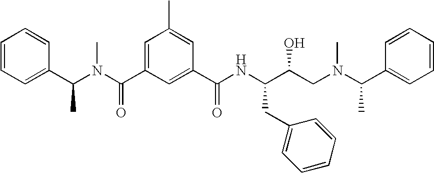

N1-((2S,3R)-3-hydroxy-4-(methyl((S)-1-phenylethyl)amino)-1-phenylbutan-2-yl)-N3,5-dimethyl-N3-((S)-1-phenylethyl)isophthalamide. m/z: 578.3.

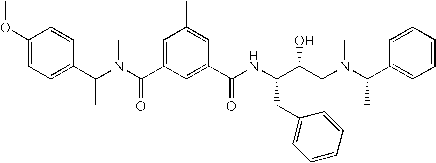

N1-((2S,3R)-3-hydroxy-4-(methyl((S)-1-phenylethyl)amino)-1-phenylbutan-2-yl)-N3-(1-(4-methoxyphenyl)ethyl)-N3,5-dimethylisophthalamide. m/z: 608.3.

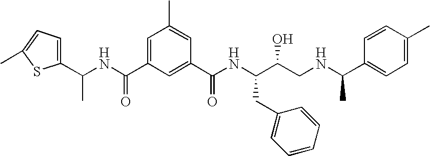

N1-((2S,3R)-3-hydroxy-1-phenyl-4-((R)-1-p-tolylethylamino)butan-2-yl)-5-methyl-N3-(1-(5-methylthiophen-2-yl)ethyl)isophthalamide. m/z: 584.3.

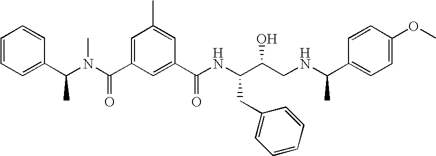

N1-((2S,3R)-3-hydroxy-4-((R)-1-(4-methoxyphenyl)ethylamino)-1-phenylbutan-2-yl)-N3,5-dimethyl-N3-((S)-1-phenylethyl)isophthalamide. m/z: 594.3.

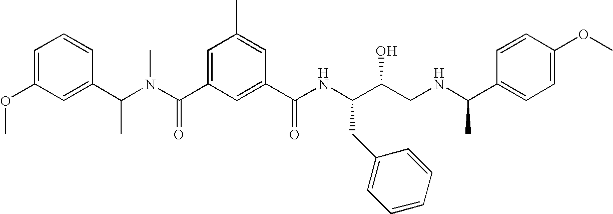

N1-((2S,3R)-3-hydroxy-4-((R)-1-(4-methoxyphenyl)ethylamino)-1-phenylbutan-2-yl)-N3-(1-(3-methoxyphenyl)ethyl)-N3,5-dimethylisophthalamide. m/z: 624.3.

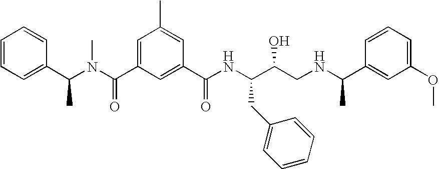

N1-((2S,3R)-3-hydroxy-4-((R)-1-(3-methoxyphenyl)ethylamino)-1-phenylbutan-2-yl)-N3,5-dimethyl-N3-((S)-1-phenylethyl)isophthalamide. m/z: 594.3.

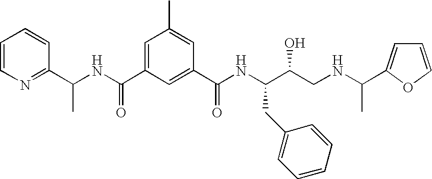

N1-((2S,3R)-4-(1-(furan-2-yl)ethylamino)-3-hydroxy-1-phenylbutan-2-yl)-5-methyl-N3-(1-(pyridin-2-yl)ethyl)isophthalamide. m/z: 541.3.

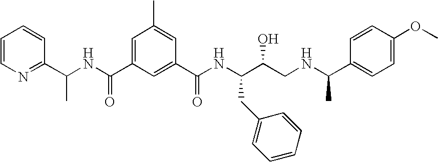

N1-((2S,3R)-3-hydroxy-4-((R)-1-(4-methoxyphenyl)ethylamino)-1-phenylbutan-2-yl)-5-methyl-N3-(1-(pyridin-2-yl)ethyl)isophthalamide. m/z: 581.3.

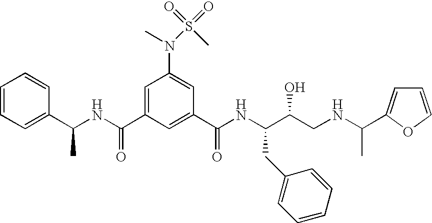

N1-((2S,3R)-4-(1-(furan-2-yl)ethylamino)-3-hydroxy-1-phenylbutan-2-yl)-5-(N-methylmethylsulfonamido)-N3-((S)-1-phenylethyl)isophthalamide. m/z: 633.3.

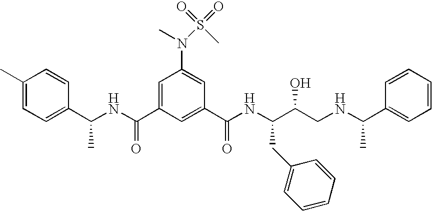

N1-((2S,3R)-3-hydroxy-1-phenyl-4-((S)-1-phenylethylamino)butan-2-yl)-5-(N-methylmethylsulfonamido)-N3-((R)-1-p-tolylethyl)isophthalamide. m/z: 657.3.

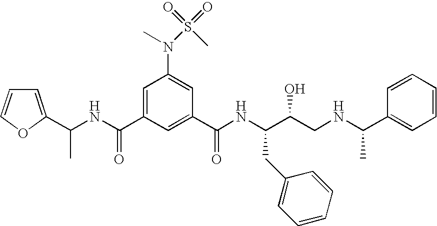

N1-(1-(furan-2-yl)ethyl)-N3-((2S,3R)-3-hydroxy-1-phenyl-4-((S)-1-phenylethylamino)butan-2-yl)-5-(N-methylmethylsulfonamido)isophthalamide. m/z: 633.3.

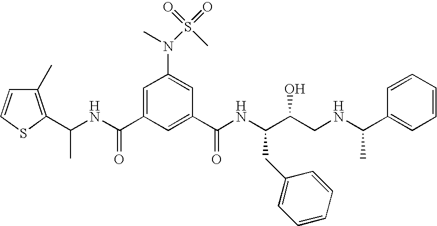

N1-((2S,3R)-3-hydroxy-1-phenyl-4-((S)-1-phenylethylamino)butan-2-yl)-5-(N-methylmethylsulfonamido)-N3-(1-(3-methylthiophen-2-yl)ethyl)isophthalamide. m/z: 663.3.

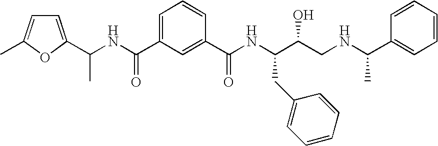

N1-((2S,3R)-3-hydroxy-1-phenyl-4-((S)-1-phenylethylamino)butan-2-yl)-N3-(1-(5-methylfuran-2-yl)ethyl)isophthalamide. m/z: 540.3.

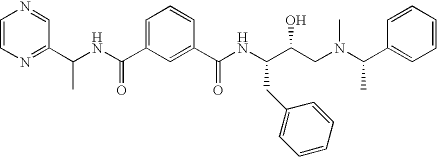

N1-((2S,3R)-3-hydroxy-4-(methyl((S)-1-phenylethyl)amino)-1-phenylbutan-2-yl)-N3-(1-(pyrazin-2-yl)ethyl)isophthalamide. m/z: 552.3.

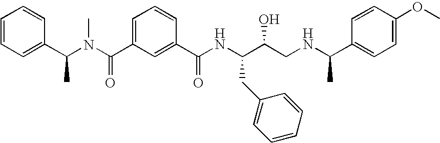

N1-((2S,3R)-3-hydroxy-4-((R)-1-(4-methoxyphenyl)ethylamino)-1-phenylbutan-2-yl)-N3-methyl-N3-((S)-1-phenylethyl)isophthalamide. m/z: 580.3.

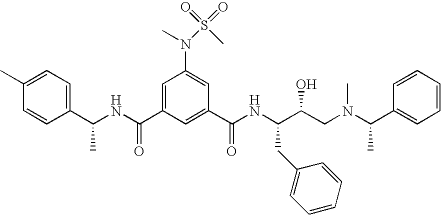

N1-((2S,3R)-3-hydroxy-4-(methyl((S)-1-phenylethyl)amino)-1-phenylbutan-2-yl)-5-(N-methylmethylsulfonamido)-N3-((R)-1-p-tolylethyl)isophthalamide. m/z: 671.3.

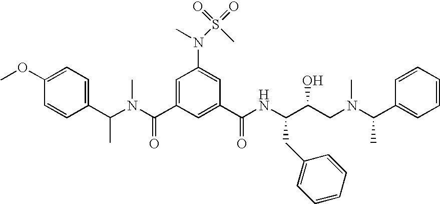

N1-((2S,3R)-3-hydroxy-4-(methyl((S)-1-phenylethyl)amino)-1-phenylbutan-2-yl)-N3-(1-(4-methoxyphenyl)ethyl)-N3-methyl-5-(N-methylmethylsulfonamido)isophthalamide. m/z: 701.2.

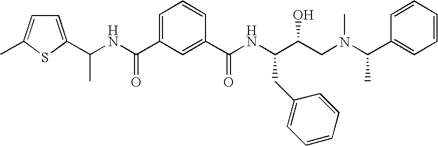

N1-((2S,3R)-3-hydroxy-4-(methyl((S)-1-phenylethyl)amino)-1-phenylbutan-2-yl)-N3-(1-(5-methylthiophen-2-yl)ethyl)isophthalamide. m/z: 570.3.

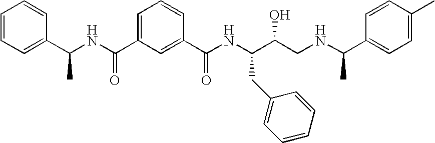

N1-((2S,3R)-3-hydroxy-1-phenyl-4-((R)-1-p-tolylethylamino)butan-2-yl)-N3-((S)-1-phenylethyl)isophthalamide. m/z: 550.3.

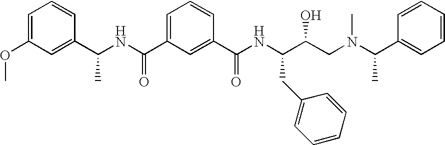

N1-((2S,3R)-3-hydroxy-4-(methyl((S)-1-phenylethyl)amino)-1-phenylbutan-2-yl)-N3-((R)-1-(3-methoxyphenyl)ethyl)isophthalamide. m/z: 580.3.

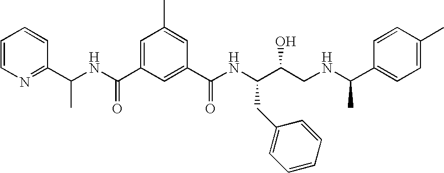

N1-((2S,3R)-3-hydroxy-1-phenyl-4-((R)-1-p-tolylethylamino)butan-2-yl)-5-methyl-N3-(1-(pyridin-2-yl)ethyl)isophthalamide. m/z: 565.3.

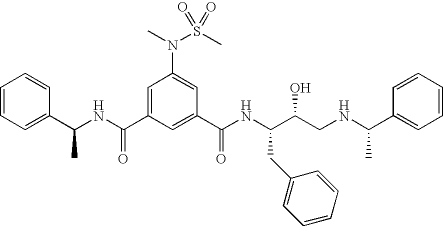

N1-((2S,3R)-3-hydroxy-1-phenyl-4-((S)-1-phenylethylamino)butan-2-yl)-5-(N-methylmethylsulfonamido)-N3-((S)-1-phenylethyl)isophthalamide. m/z: 643.3.

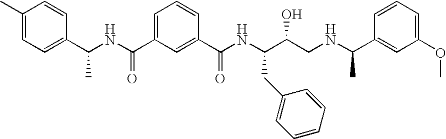

N1-((2S,3R)-3-hydroxy-4-((R)-1-(3-methoxyphenyl)ethylamino)-1-phenylbutan-2-yl)-N3-((R)-1-p-tolylethyl)isophthalamide. m/z: 580.3.

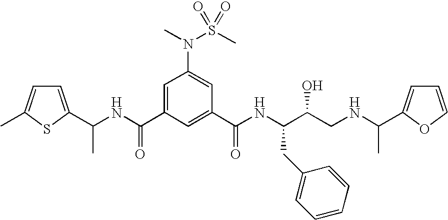

N1-((2S,3R)-4-(1-(furan-2-yl)ethylamino)-3-hydroxy-1-phenylbutan-2-yl)-5-(N-methylmethylsulfonamido)-N3-(1-(5-methylthiophen-2-yl)ethyl)isophthalamide. m/z: 653.3.

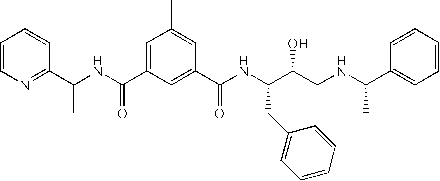

N1-((2S,3R)-3-hydroxy-1-phenyl-4-((S)-1-phenylethylamino)butan-2-yl)-5-methyl-N3-(1-(pyridin-2-yl)ethyl)isophthalamide. m/z: 551.2.

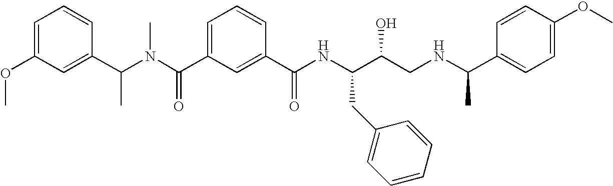

N1-((2S,3R)-3-hydroxy-4-((R)-1-(4-methoxyphenyl)ethylamino)-1-phenylbutan-2-yl)-N3-(1-(3-methoxyphenyl)ethyl)-N3-methylisophthalamide. m/z: 610.3.

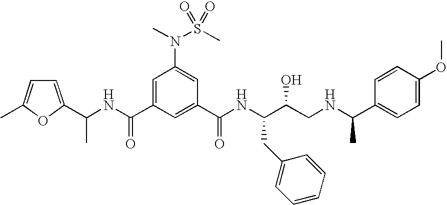

N1-((2S,3R)-3-hydroxy-4-((R)-1-(4-methoxyphenyl)ethylamino)-1-phenylbutan-2-yl)-N3-(1-(5-methylfuran-2-yl)ethyl)-5-(N-methylmethylsulfonamido)isophthalamide. m/z: 676.8.

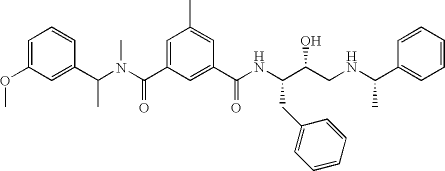

N1-((2S,3R)-3-hydroxy-1-phenyl-4-((S)-1-phenylethylamino)butan-2-yl)-N3-(1-(3-methoxyphenyl)ethyl)-N3,5-dimethylisophthalamide. m/z: 593.9.

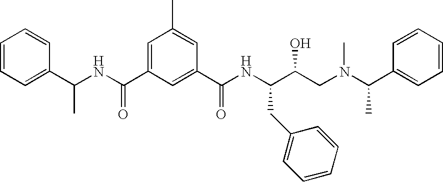

N1-((2S,3R)-3-hydroxy-4-(methyl((S)-1-phenylethyl)amino)-1-phenylbutan-2-yl)-5-methyl-N3-((S)-1-phenylethyl)isophthalamide. m/z: 564.3.

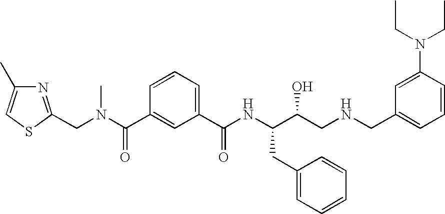

N1-((2S,3R)-4-(3-(diethylamino)benzylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.79 (m, 1H), 7.70-7.44 (m, 2H), 7.42-7.39 (m, 1H), 7.35-7.16 (m, 8H), 6.91 (broad s, 1H), 6.65-6.60 (m, 3H), 5.00 (broad s, 1.2H), 4.67 (broad s, 0.5H), 4.49-4.40 (m, 1H), 3.87-3.74 (m, 3H), 3.40-3.33 (m, 4H), 3.20-2.95 (m, 7H), 2.90-2.86 (m, 3H), 2.48 (broad s, 3H), 1.19-1.15 (m, 6H).

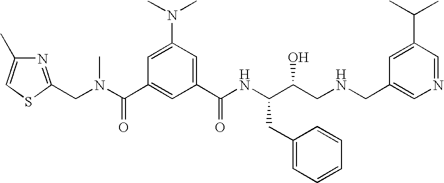

5-(dimethylamino)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.41 (s, 2H), 7.56-7.54 (m, 1H), 7.32-7.24 (m, 7H), 7.04-6.85 (m, 4H), 6.53 (m, 1H), 4.99 (broad s, 1.0H), 4.69 (broad s, 0.8H), 4.39-4.35 (m, 1H), 3.84 (s, 2H), 3.68 (m, 1H), 3.14-2.90 (m, 11H), 2.79 (m, 2H), 2.48 (broad s, 3H), 1.29-1.23 (m, 7H).

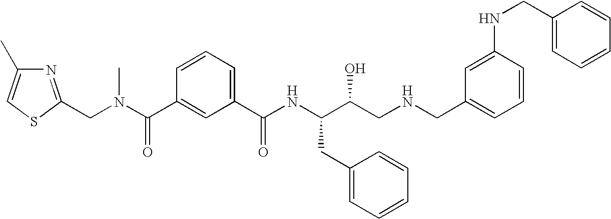

N1-((2S,3R)-4-(3-(benzylamino)benzylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.78 (m, 1H), 7.70-7.67 (m, 1H), 7.55 (m, 1H), 7.43-7.12 (m, 14H), 6.91 (broad s, 1H), 6.69-6.64 (m, 2H), 6.57-6.54 (m, 1H), 4.98 (broad s, 1.3H), 4.66 (broad s, 0.6H), 4.45-4.40 (m, 1H), 4.33 (s, 2H), 4.19-4.12 (m, 1H), 3.81-3.70 (m, 3H), 3.14-3.01 (m, 5H), 2.84 (m, 2H), 2.48 (broad s, 3H).

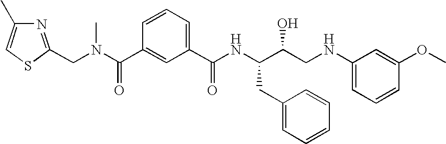

N1-((2S,3R)-3-hydroxy-4-(3-methoxyphenylamino)-1-phenylbutan-2-yl)-N3-methyl-N3-((4-methylthiazol-2-yl)methyl)isophthalamide: ¹H NMR (300 MHz, CDCl₃) δ 7.78-7.52 (m, 3H), 7.42-7.37 (m, 1H), 7.32-7.22 (m, 6H), 7.13-7.07 (m, 1H), 6.92-6.84 (m, 2H), 6.35-6.26 (m, 3H), 4.97 (broad s, 1.3H), 4.63 (broad s, 0.6H), 4.45-4.42 (m, 1H), 3.94 (m, 2H), 3.78 (s, 3H), 3.38-3.35 (m, 1H), 3.24-2.99 (m, 6H), 2.48 (broad s, 3H).

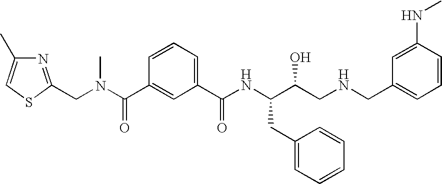

N1-((2S,3R)-3-hydroxy-4-(3-(methylamino)benzylamino)-1-phenylbutan-2-yl)-N3-methyl-N3-((4-methylthiazol-2-yl)methyl)isophthalamide: ¹H NMR (300 MHz, CDCl₃) δ 7.79-7.69 (m, 3H), 7.55 (m, 1H), 7.44-7.39 (m, 2H), 7.35-7.13 (m, 9H), 6.92 (broad s, 1H), 6.68-6.63 (m, 2H), 6.55-6.52 (m, 1H), 4.99 (broad s, 1.3H), 4.67 (broad s, 0.5H), 4.41 (m, 1H), 3.86-3.70 (m, 4H), 3.21-2.93 (m, 5H), 2.92-2.82 (m, 5H), 2.48 (broad s, 3H).

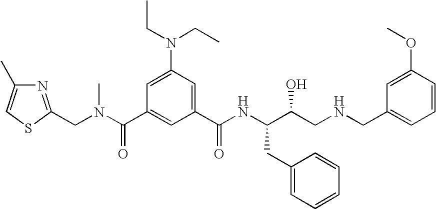

5-(diethylamino)-N1-((2S,3R)-3-hydroxy-4-(3-methoxybenzylamino)-1-phenylbutan-2-yl)-N3-methyl-N3-((4-methylthiazol-2-yl)methyl)isophthalamide: ¹H NMR (300 MHz, CDCl₃) δ 7.31-7.15 (m, 7H), 7.02 (m, 1H), 6.94-6.80 (m, 6H), 6.60 (broad s, 1H), 4.99 (broad s, 1.0H), 4.71 (broad s, 0.8H), 4.43-4.33 (m, 1H), 3.87-3.76 (m, 5H), 3.66 (broad s, 1H), 3.37 (broad s, 4H), 3.13-3.03 (m, 6H), 2.79 (d, 2H), 2.48 (broad s, 3H), 1.15 (m, 6H).

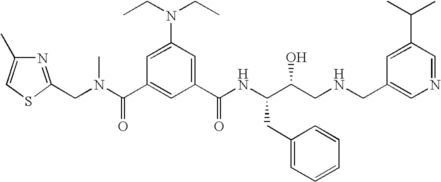

5-(diethylamino)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.39 (s, 2H), 7.55 (m, 1H), 7.32-7.23 (m, 6H), 7.02 (m, 1H), 6.89-6.78 (m, 3H), 6.59 (m, 1H), 4.98 (broad s, 1.0H), 4.72 (broad s, 0.8H), 4.41-4.32 (m, 1H), 3.82 (m, 2H), 3.69 (broad s, 1H), 3.37 (broad s, 4H), 3.13-2.84 (m, 6H), 2.78 (m, 2H), 2.47 (broad s, 3H), 1.27 (d, 7H), 1.16 (m, 6H).

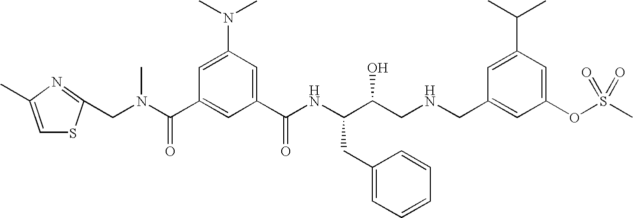

3-(((2R,3S)-3-(3-(dimethylamino)-5-(methyl((4-methylthiazol-2-yl)methyl)carbamoyl)benzamido)-2-hydroxy-4-phenylbutylamino)methyl)-5-isopropylphenyl methanesulfonate: ¹H NMR (300 MHz, CDCl₃) δ 7.33-7.13 (m, 6H), 7.05-6.86 (m, 3H), 4.98 (broad s, 1.0H), 4.68 (broad s, 0.7H), 4.41-4.34 (m, 1H), 3.84 (m, 2H), 3.70-3.68 (m, 1H), 3.20-3.09 (m, 3H), 3.07-2.88 (m, 10H), 2.78 (m, 2H), 2.48 (broad s, 3H), 1.26 (d, 7H).

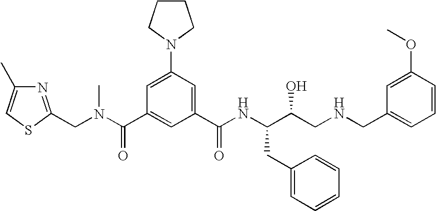

N1-((2S,3R)-3-hydroxy-4-(3-methoxybenzylamino)-1-phenylbutan-2-yl)-N3-methyl-N3-((4-methylthiazol-2-yl)methyl)-5-(pyrrolidin-1-yl)isophthalamide: ¹H NMR (300 MHz, CDCl₃) δ 7.31-7.12 (m, 6H), 6.94-6.67 (m, 7H), 4.98 (broad s, 1.0H), 4.70 (broad s, 0.7H), 4.42-4.33 (m, 1H), 3.87-3.76 (m, 4H), 3.67 (m, 1H), 3.28 (broad s, 4H), 3.13-3.01 (m, 6H), 2.80 (d, 2H), 2.48 (broad s, 3H), 2.03 (broad s, 4H).

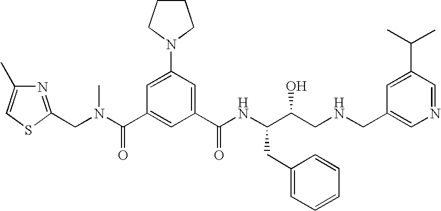

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyrrolidin-1-yl)isophthalamide: ¹H NMR (300 MHz, CDCl₃) δ 8.39 (broad s, 2H), 7.56-7.54 (m, 1H), 7.31-7.21 (m, 5H), 6.91-6.81 (m, 3H), 6.67 (m, 2H), 4.98 (broad s, 1.1H), 4.70 (broad s, 0.7H), 4.39-4.35 (m, 1H), 3.82 (s, 2H), 3.69 (m, 1H), 3.28 (broad s, 4H), 3.13-2.89 (m, 6H), 2.79 (m, 2H), 2.48 (broad s, 3H), 2.07 (broad s, 4H), 1.28 (d, 7H).

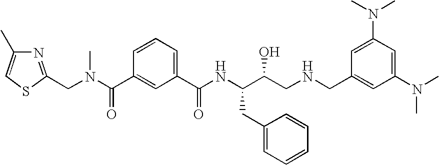

N1-((2S,3R)-4-(3,5-bis(dimethylamino)benzylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.89-7.81 (m, 1H), 7.75-7.71 (m, 1H), 7.58-7.54 (m, 2H), 7.41-7.36 (m, 1H), 7.32-7.14 (m, 5H), 6.91 (broad s, 1H), 6.19 (d, 2H), 6.01-5.99 (m, 1H), 4.98 (broad s, 1.2H), 4.66 (broad s, 0.5H), 4.45-4.36 (m, 1H), 3.88-3.68 (m, 5H), 3.16-3.12 (m, 1H), 3.08-2.83 (m, 17H), 2.48 (broad s, 3H).

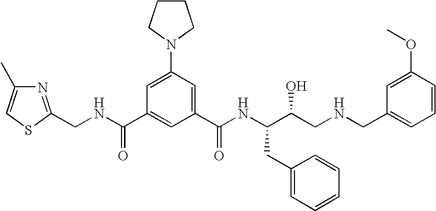

N1-((2S,3R)-3-hydroxy-4-(3-methoxybenzylamino)-1-phenylbutan-2-yl)-N3-((4-methylthiazol-2-yl)methyl)-5-(pyrrolidin-1-yl)isophthalamide: ¹H NMR (300 MHz, CDCl₃) δ 7.78 (m, 1H), 7.35-7.12 (m, 10H), 6.91-6.79 (m, 5H), 4.86-4.84 (m, 2H), 4.51-4.36 (m, 1H), 3.87-3.71 (m, 6H), 3.23 (m, 4H), 2.98 (d, 3H), 2.84-2.82 (m, 3H), 2.38 (s, 3H), 2.00 (m, 4H).

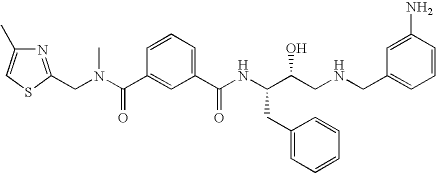

N1-((2S,3R)-4-(3-aminobenzylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD) δ 7.73-7.69 (m, 2H), 7.55-7.53 (m, 1H), 7.45-7.39 (m, 1H), 7.30-7.06 (m, 7H), 6.92 (broad s, 1H), 6.70-6.67 (m, 2H), 6.60-6.58 (m, 1H), 4.97 (broad s, 1.2H), 4.67 (broad s, 0.5H), 4.34 (m, 1H), 3.84-3.66 (m, 3H), 3.12-2.79 (m, 6H), 2.46 (broad s, 3H).

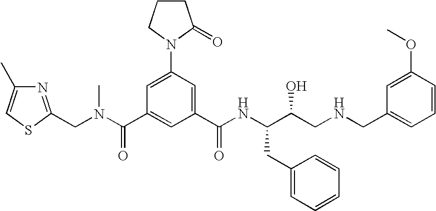

N1-((2S,3R)-3-hydroxy-4-(3-methoxybenzylamino)-1-phenylbutan-2-yl)-N3-methyl-N3-((4-methylthiazol-2-yl)methyl)-5-(2-oxopyrrolidin-1-yl)isophthalamide: ¹H NMR (300 MHz, CDCl₃) δ 8.06-7.87 (m, 2H), 7.58-7.49 (m, 2H), 7.32-7.17 (m, 7H), 6.94-6.91 (m, 3H), 6.83-6.80 (m, 1H), 4.98 (m, 1.3H), 4.68 (broad s, 0.6H), 4.43-4.38 (m, 1H), 3.89-3.73 (m, 8H), 3.13-3.00 (m, 7H), 2.84-2.83 (m, 2H), 2.66-2.60 (m, 2H), 2.48 (broad s, 3H), 2.20-2.16 (m, 2H).

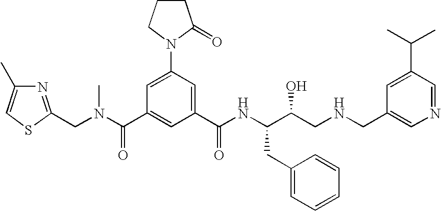

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(2-oxopyrrolidin-1-yl)isophthalamide: ¹H NMR (300 MHz, CDCl₃) δ 8.38 (s, 2H), 8.00 (m, 1H), 7.90 (broad s, 1H), 7.59-7.40 (m, 3H), 7.30-7.15 (m, 6H), 6.89 (broad s, 1H), 5.03-4.91 (m, 1.3H), 4.67 (broad s, 0.5H), 4.42-4.35 (m, 1H), 3.91-3.70 (m, 5H), 3.13-2.82 (m, 10H), 2.65-2.60 (m, 2H), 2.47 (broad s, 3H), 2.23-2.16 (m, 2H), 1.25 (d, 7H).

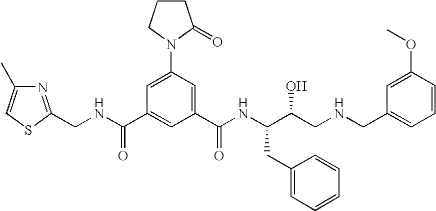

N1-((2S,3R)-3-hydroxy-4-(3-methoxybenzylamino)-1-phenylbutan-2-yl)-N3-((4-methylthiazol-2-yl)methyl)-5-(2-oxopyrrolidin-1-yl)isophthalamide: ¹H NMR (300 MHz, CDCl₃) δ 8.75 (broad s, 1H), 8.27-8.22 (m, 2H), 7.92 (d, 1H), 7.30-7.12 (m, 7H), 6.96-6.93 (m, 2H), 6.83-6.79 (m, 2H), 4.96-4.75 (m, 2H), 4.40-4.37 (m, 1H), 3.98-3.73 (m, 7H), 3.68-3.63 (m, 2H), 3.04-2.85 (m, 4H), 2.65-2.58 (m, 2H), 2.35 (d, 3H), 2.16-2.06 (m, 3H).

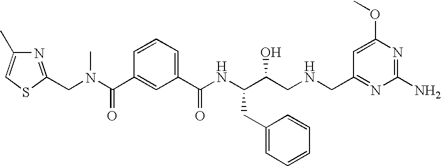

N1-((2S,3R)-4-((2-amino-6-methoxypyrimidin-4-yl)methylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.79-7.70 (m, 2H), 7.58 (m, 1H), 7.45-7.40 (m, 1H), 7.30-7.16 (m, 6H), 6.92 (broad s, 1H), 6.05 (s, 1H), 5.08 (broad s, 2H), 4.99 (broad s, 1.3H), 4.68 (broad s, 0.5H), 4.53-4.44 (m, 1H), 3.89 (s, 3H), 3.75-3.68 (m, 3H), 3.14-3.02 (m, 5H), 2.86-2.84 (m, 3H), 2.48 (broad s, 3H).

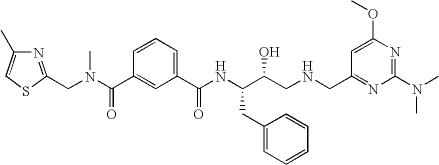

N1-((2S,3R)-4-((2-(dimethylamino)-6-methoxypyrimidin-4-yl)methylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.79 (m, 1H), 7.71-7.68 (m, 1H), 7.60-7.57 (m, 1H), 7.44-7.39 (m, 1H), 7.32-7.18 (m, 6H), 6.92 (broad s, 1H), 5.90 (s, 1H), 5.00 (broad s, 1.3H), 4.67 (broad s, 0.6H), 4.53-4.44 (m, 1H), 3.92 (s, 3H), 3.75-3.65 (m, 3H), 3.17-3.14 (m, 6H), 3.08-2.98 (m, 4H), 2.90-2.89 (m, 2H), 2.48 (broad s, 3H).

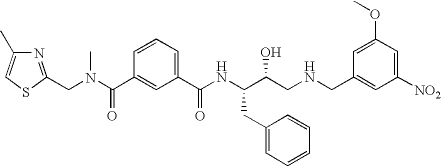

N1-((2S,3R)-3-hydroxy-4-(3-methoxy-5-nitrobenzylamino)-1-phenylbutan-2-yl)-N3-methyl-N3-((4-methylthiazol-2-yl)methyl)isophthalamide: ¹H NMR (300 MHz, CDCl₃) δ 7.82-7.73 (m, 2H), 7.68-7.55 (m, 3H), 7.44-7.39 (m, 1H), 7.31-7.19 (m, 7H), 6.92 (m, 2H), 4.99 (broad s, 1.2H), 4.66 (broad s, 0.6H), 4.42-4.35 (m, 1H), 3.94-3.83 (m, 5H), 3.72-3.71 (m, 1H), 3.14-2.97 (m, 5H), 2.85-2.75 (m, 2H), 2.48 (broad s, 3H).

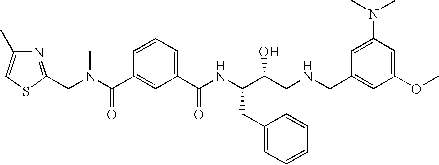

N1-((2S,3R)-4-(3-(dimethylamino)-5-methoxybenzylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.78 (m, 1H), 7.69-7.58 (m, 2H), 7.45-7.40 (m, 1H), 7.32-7.20 (m, 7H), 6.92 (broad s, 1H), 6.34-6.30 (m, 2H), 6.21-6.19 (m, 1H), 5.00 (broad s, 1.2H), 4.68 (broad s, 0.5H), 4.47-4.42 (m, 1H), 3.84-3.71 (m, 6H), 3.16-3.03 (m, 5H), 2.95-2.93 (m, 6H), 2.87 (m, 2H), 2.48 (broad s, 3H).

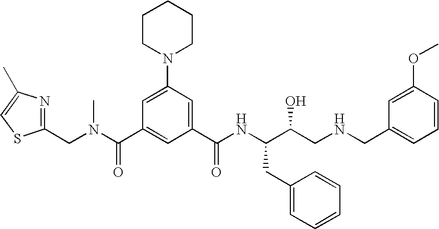

N1-((2S,3R)-3-hydroxy-4-(3-methoxybenzylamino)-1-phenylbutan-2-yl)-N3-methyl-N3-((4-methylthiazol-2-yl)methyl)-5-(piperidin-1-yl)isophthalamide: ¹H NMR (300 MHz, CDCl₃) δ 7.31-7.19 (m, 9H), 7.07 (broad s, 1H), 6.97-5-6.80 (m, 5H), 6.68-6.58 (m, 1H), 4.98 (broad s, 1.2H), 4.68 (broad s, 0.8H), 4.40-4.36 (m, 1H), 3.88-3.77 (m, 5H), 3.67 (m, 1H), 3.21-3.01 (m, 9H), 2.80-2.79 (m, 2H), 2.48 (broad s, 3H), 1.68-1.64 (m, 6H).

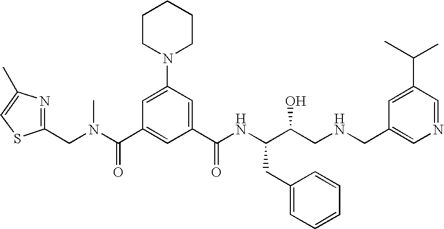

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(piperidin-1-yl)isophthalamide: ¹H NMR (300 MHz, CDCl₃) δ 8.41-8.39 (m, 2H), 7.55-7.54 (m, 1H), 7.37-7.19 (m, 7H), 7.07 (broad s, 1H), 6.96-6.92 (m, 1H), 6.57 (m, 1H), 4.98 (broad s, 1.1H), 4.68 (broad s, 0.8H), 4.41-4.32 (m, 1H), 3.83 (m, 2H), 3.68 (m, 1H), 3.21-2.90 (m, 9H), 2.84-2.78 (m, 2H), 2.48 (broad s, 3H), 1.68-1.64 (m, 6H), 1.28 (d, 6H).

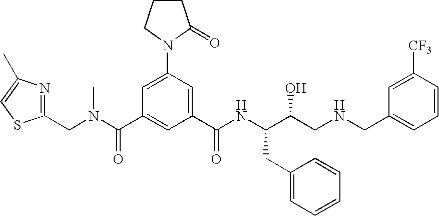

N1-((2S,3R)-3-hydroxy-1-phenyl-4-(3-(trifluoromethyl)benzylamino)butan-2-yl)-N3-methyl-N3-((4-methylthiazol-2-yl)methyl)-5-(2-oxopyrrolidin-1-yl)isophthalamide: ¹H NMR (300 MHz, CDCl₃) δ 8.07-7.94 (m, 2H), 7.64-7.43 (m, 5H), 7.35-7.18 (m, 6H), 6.91 (m, 1H), 5.04-4.93 (m, 1.3H), 4.68 (broad s, 0.6H), 4.45-4.38 (m, 1H), 3.96-3.73 (m, 5H), 3.14-3.01 (m, 5H), 2.82-2.81 (m, 2H), 2.67-2.62 (m, 2H), 2.48 (broad s, 3H), 2.24-2.17 (m, 3H).

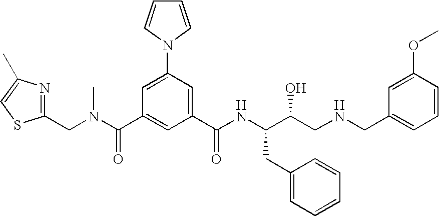

N1-((2S,3R)-3-hydroxy-4-(3-methoxybenzylamino)-1-phenylbutan-2-yl)-N3-methyl-N3-((4-methylthiazol-2-yl)methyl)-5-(1H-pyrrol-1-yl)isophthalamide: ¹H NMR (300 MHz, CDCl₃) δ 7.81-7.70 (m, 1H), 7.64-7.49 (m, 1H), 7.32-7.19 (m, 8H), 7.08 (s, 2H), 6.95-6.90 (m, 3H), 6.83-6.79 (m, 1H), 6.38 (s, 2H), 5.00 (broad s, 1.2H), 4.66 (broad s, 0.7H), 4.47-4.38 (m, 1H), 3.89-3.77 (m, 6H), 3.16-3.03 (m, 5H), 2.84 (m, 2H), 2.49 (s, 4H).

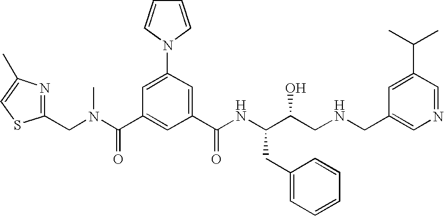

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrrol-1-yl)isophthalamide: ¹H NMR (300 MHz, CDCl₃) δ 8.41 (s, 2H), 7.86-7.71 (m, 1H), 7.61-7.48 (m, 2H), 7.33-7.21 (m, 7H), 7.10-7.09 (m, 2H), 6.94 (m, 2H), 6.39 (s, 2H), 5.01 (broad s, 1.1H), 4.69 (broad s, 0.7H), 4.47-4.40 (m, 1H), 3.90-3.74 (m, 3H), 3.18-3.06 (m, 5H), 2.99-2.90 (m, 1H), 2.83-2.81 (m, 2H), 2.49 (s, 3H), 1.27 (d, 6H).

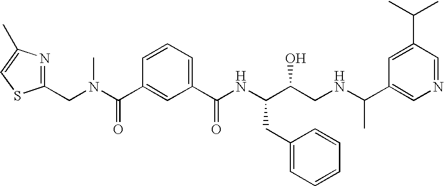

N1-((2S,3R)-3-hydroxy-4-(1-(5-isopropylpyridin-3-yl)ethylamino)-1-phenylbutan-2-yl)-N3-methyl-N3-((4-methylthiazol-2-yl)methyl)isophthalamide: ¹H NMR (300 MHz, CDCl₃): 1.20 (d, J=6.9 Hz, 6H), 1.25 (d, J=6.9 Hz, 6H), 1.40 (d, J=6.3 Hz, 3H), 1.44 (d, J=6.6 Hz, 3H), 2.45 (s, 3H), 2.47 (s, 3H), 2.50-2.78 (m, 4H), 2.74-3.20 (m, 12H), 3.60-3.72 (m, 2H), 3.72-3.82 (m, 2H), 4.26-4.46 (m, 2H), 4.84-5.40 (m, 4H), 6.86-6.94 (m, 2H), 7.10-7.32 (m, 10H), 7.40-7.48 (m, 2H), 7.48-7.64 (m, 4H), 7.64-7.90 (m, 4H), 8.24-8.37 (m, 4H).

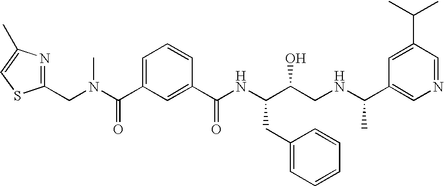

N1-((2S,3R)-3-hydroxy-4-((S)-1-(5-isopropylpyridin-3-yl)ethylamino)-1-phenylbutan-2-yl)-N3-methyl-N3-((4-methylthiazol-2-yl)methyl)isophthalamide: ¹H NMR (300 MHz, CDCl₃): 1.25 (d, J=6.9 Hz, 6H), 1.40 (d, J=6.3 Hz, 3H), 2.45 (s, 3H), 2.50-2.60 (m, 1H), 2.62-2.74 (m, 1H), 2.74-3.18 (m, 6H), 3.60-3.70 (m, 1H), 3.72-3.82 (m, 1H), 4.26-4.38 (m, 1H), 4.84-5.40 (m, 2H), 6.86-6.94 (m, 1H), 7.10-7.28 (m, 5H), 7.40-7.48 (m, 1H), 7.50-7.60 (m, 2H), 7.64-7.74 (m, 2H), 8.24-8.37 (m, 2H).

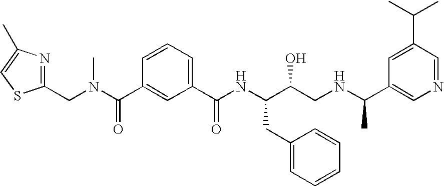

N1-((2S,3R)-3-hydroxy-4-((R)-1-(5-isopropylpyridin-3-yl)ethylamino)-1-phenylbutan-2-yl)-N3-methyl-N3-((4-methylthiazol-2-yl)methyl)isophthalamide: ¹H NMR (300 MHz, CDCl₃): 1.20 (d, J=6.9 Hz, 6H), 1.44 (d, J=6.6 Hz, 3H), 2.47 (s, 3H), 2.56-2.66 (m, 1H), 2.66-2.78 (m, 1H), 2.78-3.20 (m, 6H), 3.62-3.72 (m, 1H), 3.72-3.82 (m, 1H), 4.36-4.46 (m, 1H), 4.90 (br s, 2H), 6.84 (br s, 1H), 7.12-7.32 (m, 5H), 7.40-7.48 (m, 1H), 7.48-7.64 (m, 2H), 7.74-7.90 (m, 2H), 8.37 (br s, 2H).

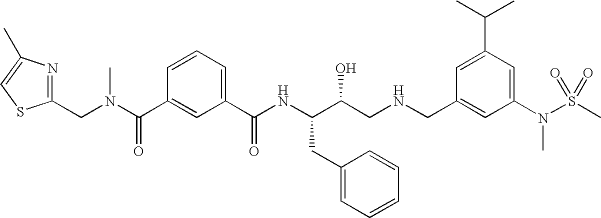

N1-((2S,3R)-3-hydroxy-4-(3-isopropyl-5-(N-methylmethylsulfonamido)benzylamino)-1-phenylbutan-2-yl)-N3-methyl-N3-((4-methylthiazol-2-yl)methyl)isophthalamide: ¹H NMR (300 MHz, CDCl₃) δ 7.73-7.85 (m, 1H), 7.68-7.71 (m, 1H), 7.55-7.67 (m, 1H), 7.44-7.49 (m, 1H), 7.19-7.34 (m, 6H), 7.168-7.17 (m, 2H), 6.88-6.96 (m, 1H), 6.69-6.88 (m, 1H), 5.01 (s, 2H), 4.34-4.47 (m, 1H), 3.84-3.94 (m, 2H), 3.66-3.78 (m, 1H), 3.33 (s, 3H), 2.80-3.21 (m, 9H), 2.86 (s, 3H), 2.49 (s, 3H), 1.26-1.28 (m, 6H).

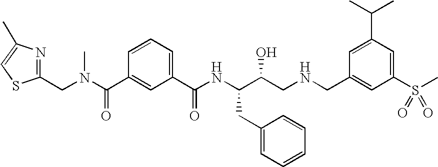

N1-((2S,3R)-3-hydroxy-4-(3-isopropyl-5-(methylsulfonyl)benzylamino)-1-phenylbutan-2-yl)-N3-methyl-N3-((4-methylthiazol-2-yl)methyl)isophthalamide: ¹H NMR (300 MHz, CDCl₃) δ 7.71-7.89 (m, 3H), 7.67-7.70 (m, 1H), 7.55-7.65 (m, 1H), 7.43-7.53 (m, 2H), 7.19-7.36 (m, 5H), 6.86-6.97 (m, 1H), 6.49-6.70 (m, 2H), 5.01 (s, 2H), 4.34-4.47 (m, 1H), 3.86-3.96 (m, 2H), 3.64-3.76 (m, 1H), 2.93-3.20 (m, 7H), 3.08 (s, 3H), 2.74-2.89 (m, 2H), 2.48 (s, 3H), 1.29-1.31 (m, 6H).

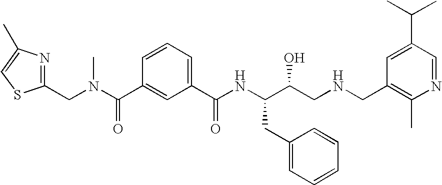

N1-((2S,3R)-3-hydroxy-4-((5-isopropyl-2-methylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.30-8.31 (m, 1H), 7.71-7.86 (m, 1H), 7.64-7.67 (m, 1H), 7.54-7.61 (m, 1H), 7.39-7.48 (m, 2H), 7.19-7.36 (m, 5H), 6.86-6.97 (m, 1H), 6.58-6.82 (m, 1H), 5.00 (s, 2H), 4.36-4.49 (m, 1H), 3.81 (s, 2H), 3.68-3.78 (m, 1H), 2.99-3.20 (m, 6H), 2.81-2.99 (m, 3H), 2.57 (s, 3H), 2.48 (s, 3H), 1.25-1.28 (m, 6H).

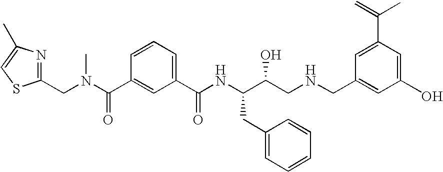

N1-((2S,3R)-3-hydroxy-4-(3-hydroxy-5-(prop-1-en-2-yl)benzylamino)-1-phenylbutan-2-yl)-N3-methyl-N3-((4-methylthiazol-2-yl)methyl)isophthalamide: ¹H NMR (300 MHz, CDCl₃) δ 7.78-7.87 (m, 1H), 7.58-7.78 (m, 2H), 7.45-7.58 (m, 1H), 7.15-7.42 (m, 7H), 6.77-6.97 (m, 4H), 5.33 (s, 1H), 4.94-5.12 (m, 2H), 5.05 (s, 1H), 4.39-4.54 (m, 1H), 3.66-3.87 (m, 3H), 2.84-3.27 (m, 8H), 2.48 (s, 3H), 2.08 (s, 3H).

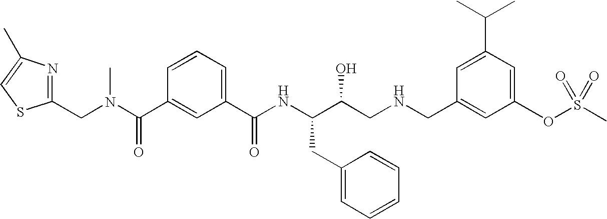

3-(((2R,3S)-2-hydroxy-3-(3-(methyl((4-methylthiazol-2-yl)methyl)carbamoyl)benzamido)-4-phenylbutylamino)methyl)-5-isopropylphenyl methanesulfonate: ¹H NMR (300 MHz, CDCl₃) δ 7.71-7.85 (m, 1H), 7.66-7.69 (m, 1H), 7.52-7.62 (m, 1H), 7.41-7.46 (m, 1H), 7.19-7.33 (m, 5H), 7.15-7.19 (m, 1H), 7.11-7.15 (m, 1H), 7.04-7.07 (m, 1H), 6.73-6.97 (m, 2H), 4.99 (s, 2H), 4.34-4.46 (m, 1H), 3.79-3.90 (m, 2H), 3.63-3.74 (m, 1H), 2.86-3.20 (m, 7H), 3.15 (s, 3H), 2.74-2.86 (m, 2H), 2.48 (m, 3H), 1.24-1.27 (m, 6H).

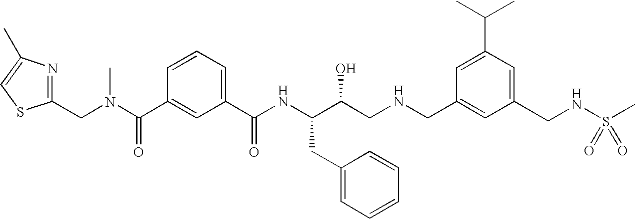

N1-((2S,3R)-3-hydroxy-4-(3-isopropyl-5-(methylsulfonamidomethyl)benzylamino)-1-phenylbutan-2-yl)-N3-methyl-N3-((4-methylthiazol-2-yl)methyl)isophthalamide: ¹H NMR (300 MHz, CDCl₃) δ 7.77-7.94 (m, 2H), 7.50-7.69 (m, 3H), 7.37-7.50 (m, 2H), 7.13-7.37 (m, 5H), 7.04-7.11 (m, 1H), 6.87-6.98 (m, 1H), 4.99 (s, 2H), 4.44-4.59 (m, 2H), 4.23-4.40 (m, 3H), 3.91-4.09 (m, 1H), 3.65-3.75 (m, 1H), 3.50-3.64 (m, 2H), 2.74-3.24 (m, 11H), 2.48 (s, 3H), 1.24-1.27 (m, 6H).

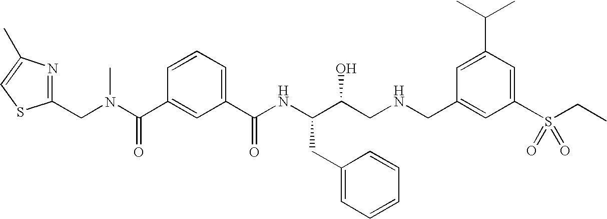

N1-((2S,3R)-4-(3-(ethylsulfonyl)-5-isopropylbenzylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.64-7.89 (m, 4H), 7.52-7.64 (m, 1H), 7.50 (s, 1H), 7.41-7.46 (m, 1H), 7.17-7.33 (m, 5H), 6.71-6.95 (m, 2H), 4.99 (s, 2H), 4.34-4.46 (m, 1H), 3.84-3.94 (m, 2H), 3.66-3.76 (m, 1H), 2.94-3.19 (m, 9H), 2.74-2.86 (m, 2H), 2.48 (s, 3H), 1.27-1.32 (m, 9H).

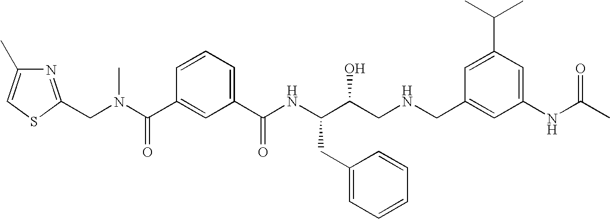

N1-((2S,3R)-4-(3-acetamido-5-isopropylbenzylamino)-3-hydroxy-1-phenylbutan-2-yl)-N3-methyl-N3-((4-methylthiazol-2-yl)methyl)isophthalamide: ¹H NMR (300 MHz, CDCl₃) δ 8.63-8.78 (m, 1H), 7.64-7.86 (m, 3H), 7.54-7.64 (m, 1H), 7.43-7.54 (m, 1H), 7.34-7.40 (m, 1H), 7.26-7.33 (m, 1H), 7.09-7.26 (m, 5H), 6.83-6.95 (m, 1H), 4.93-5.05 (m, 2H), 4.26-4.41 (m, 1H), 3.86-4.20 (m, 2H), 3.66-3.86 (m, 2H), 2.73-3.19 (m, 10H), 2.47 (s, 3H), 2.07 (s, 3H), 1.17-1.20 (m, 6H).

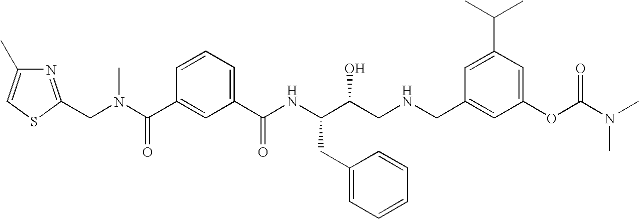

3-(((2R,3S)-2-hydroxy-3-(3-(methyl((4-methylthiazol-2-yl)methyl)carbamoyl)benzamido)-4-phenylbutylamino)methyl)-5-isopropylphenyl dimethylcarbamate: ¹H NMR (300 MHz, CDCl₃) δ 7.73-7.90 (m, 1H), 7.67-7.69 (m, 1H), 7.54-7.64 (m, 1H), 7.40-7.45 (m, 1H), 7.09-7.37 (m, 5H), 7.02 (s, 1H), 6.96-6.97 (m, 1H), 6.85-6.95 (m, 2H), 5.00 (s, 2H), 4.35-4.47 (m, 1H), 3.76-3.87 (m, 2H), 3.63-3.74 (m, 1H), 2.97-3.20 (m, 12H), 2.76-2.96 (m, 4H), 2.48 (s, 3H), 1.22-1.24 (m, 6H).

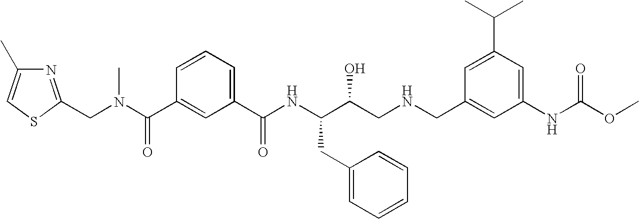

methyl 3-(((2R,3S)-2-hydroxy-3-(3-(methyl((4-methylthiazol-2-yl)methyl)carbamoyl)benzamido)-4-phenylbutylamino)methyl)-5-isopropylphenylcarbamate: ¹H NMR (300 MHz, CDCl₃) δ 7.73-7.88 (m, 1H), 7.67-7.70 (m, 1H), 7.51-7.59 (m, 1H), 7.36-7.41 (m, 1H), 7.13-7.33 (m, 7H), 6.84-6.93 (m, 2H), 5.00 (s, 2H), 4.36-4.49 (m, 1H), 3.67-3.86 (m, 6H), 3.07-3.50 (m, 2H), 2.93-3.07 (m, 5H), 2.76-2.93 (m, 4H), 2.47 (s, 3H), 1.21-1.23 (m, 6H).

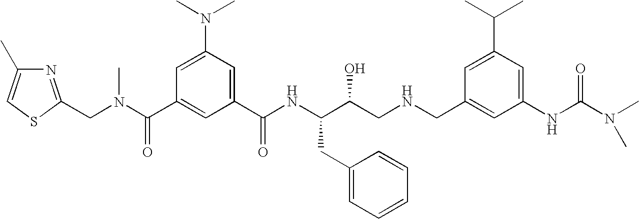

3-(((2R,3S)-3-(3-(dimethylamino)-5-(methyl((4-methylthiazol-2-yl)methyl)carbamoyl)benzamido)-2-hydroxy-4-phenylbutylamino)methyl)-5-isopropylphenyl dimethylcarbamate: ¹H NMR (300 MHz, CDCl₃) δ 7.06-7.28 (m, 7H), 6.96 (s, 1H), 6.88 (s, 1H), 6.80 (s, 1H), 6.71-6.93 (m, 4H), 4.88 (s, 2H), 4.21-4.33 (m, 1H), 3.71-3.79 (s, 2H), 3.54-3.65 (m, 1H), 2.84-3.08 (m, 18H), 2.67-2.84 (m, 3H), 2.38 (s, 3H), 1.13-1.15 (m, 6H).

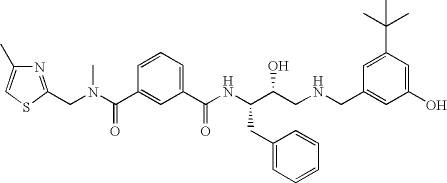

N1-((2S,3R)-4-(3-tert-butyl-5-hydroxybenzylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.78-7.91 (m, 2H), 7.67-7.70 (m, 1H), 7.43-7.63 (m, 1H), 7.12-7.38 (m, 8H), 6.86-6.95 (m, 1H), 6.73-6.86 (m, 2H), 4.94-5.05 (m, 2H), 4.35-4.49 (m, 1H), 3.86-3.98 (m, 1H), 3.69-3.86 (m, 2H), 3.09-3.21 (m, 1H), 2.93-3.09 (m, 5H), 2.83-2.93 (m, 2H), 2.48 (s, 3H), 1.24 (s, 9H).

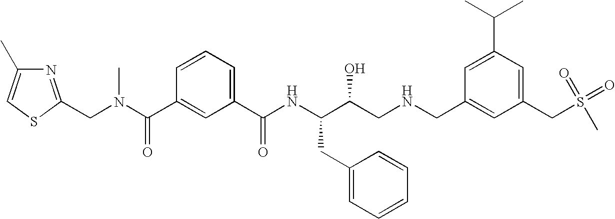

N1-((2S,3R)-3-hydroxy-4-(3-isopropyl-5-(methylsulfonylmethyl)benzylamino)-1-phenylbutan-2-yl)-N3-methyl-N3-((4-methylthiazol-2-yl)methyl)isophthalamide: ¹H NMR (300 MHz, CDCl₃) δ 7.73-7.89 (m, 1H), 7.68-7.71 (m, 1H), 7.55-7.66 (m, 1H), 7.42-7.47 (m, 1H), 7.14-7.34 (m, 8H), 6.86-7.00 (m, 1H), 5.00 (s, 2H), 4.35-4.47 (m, 1H), 4.23 (s, 2H), 3.81-3.90 (m, 2H), 3.65-3.76 (m, 1H), 2.86-3.20 (m, 8H), 2.81-2.83 (m, 2H), 2.78 (s, 3H), 2.48 (s, 3H), 1.25-1.27 (m, 6H).

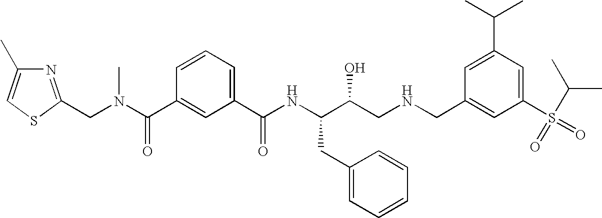

N1-((2S,3R)-3-hydroxy-4-(3-isopropyl-5-(isopropylsulfonyl)benzylamino)-1-phenylbutan-2-yl)-N3-methyl-N3-((4-methylthiazol-2-yl)methyl)isophthalamide: ¹H NMR (300 MHz, CDCl₃) δ 7.72-7.86 (m, 1H), 7.63-7.72 (m, 3H), 7.55-7.63 (m, 1H), 7.44-7.54 (m, 2H), 7.19-7.35 (m, 7H), 6.88-6.96 (m, 1H), 6.53-6.74 (m, 1H), 5.01 (s, 2H), 4.35-4.47 (m, 1H), 3.86-3.96 (m, 2H), 3.67-3.76 (m, 1H), 2.95-3.28 (m, 7H), 2.81-2.82 (m, 2H), 2.49 (s, 3H), 1.28-1.33 (m, 12H).

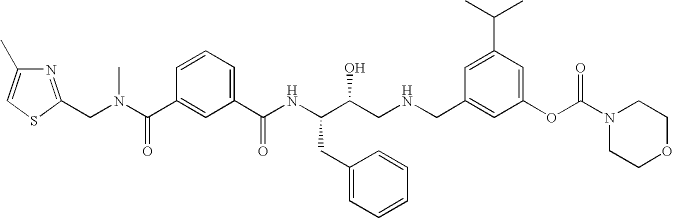

3-(((2R,3S)-2-hydroxy-3-(3-(methyl((4-methylthiazol-2-yl)methyl)carbamoyl)benzamido)-4-phenylbutylamino)methyl)-5-isopropylphenyl morpholine-4-carboxylate: ¹H NMR (300 MHz, CDCl₃) δ 7.72-7.87 (m, 1H), 7.64-7.72 (m, 1H), 7.56-7.64 (m, 1H), 7.41-7.49 (m, 1H), 7.18-7.40 (m, 7H), 7.03-7.07 (m, 1H), 6.95-7.00 (m, 1H), 6.84-6.95 (m, 3H), 5.01 (s, 2H), 4.35-4.48 (m, 1H), 3.81-3.87 (m, 2H), 3.63-3.81 (m, 7H), 3.51-3.63 (m, 2H), 2.97-3.21 (m, 5H), 2.87-2.97 (m, 1H), 2.77-2.87 (m, 2H), 2.49 (s, 3H), 1.24-1.26 (m, 6H).

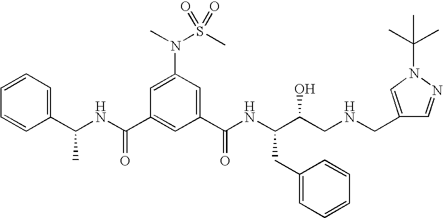

N1-((2S,3R)-4-((1-tert-butyl-1H-pyrazol-4-yl)methylamino)-3-hydroxy-1-phenylbutan-2-yl)-5-(N-methylmethylsulfonamido)-N3-((R)-1-phenylethyl)isophthalamide: ¹H NMR (300 MHz, CDCl₃) δ 8.01 (s, 1H), 7.96 (m, 1H), 7.83 (m, 1H), 7.19-7.47 (m, 12H), 5.34 (m, 1H), 4.42 (m, 1H), 3.85 (m, 1H), 3.45-3.65 (m, 2H), 3.35 (s, 3H), 2.89-2.97 (m, 2H), 2.86 (s, 3H), 2.65-2.72 (m, 1H), 2.41-2.46 (m, 1H), 1.65 (m, 3H), 1.56 (s, 9H).

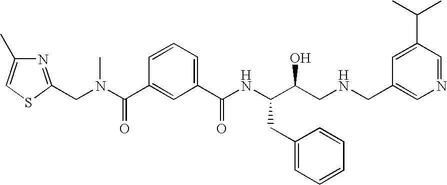

N1-((2S,3S)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.29-8.33 (m, 2H), 7.84 (s, 1H), 7.77 (m, 1H), 7.59 (m, 1H), 7.41-7.46 (m, 2H), 7.18-7.29 (m, 5H), 6.89 (s, 1H), 4.98 (m, 2H), 4.35 (m, 1H), 3.65-3.79 (m, 3H), 3.02-3.13 (m, 5H), 2.84-2.96 (m, 1H), 2.59-2.73 (m, 2H), 2.45 (s, 3H), 1.24 (m, 6H).

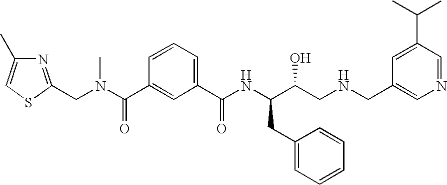

N1-((2R,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.27-8.31 (m, 2H), 7.84 (s, 1H), 7.75 (m, 1H), 7.55 (m, 1H), 7.38-7.43 (m, 2H), 7.15-7.24 (m, 5H), 6.87 (s, 1H), 4.96 (m, 2H), 4.33 (m, 1H), 3.64-3.78 (m, 3H), 3.05-3.11 (m, 5H), 2.82-2.95 (m, 1H), 2.59-2.71 (m, 2H), 2.43 (s, 3H), 1.21 (m, 6H).

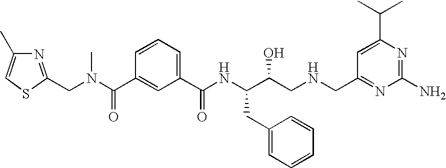

N1-((2S,3R)-4-((2-amino-6-isopropylpyrimidin-4-yl)methylamino)-3-hydroxy-1-phenylbutan-2-yl)-N3-methyl-N3-((4-methylthiazol-2-yl)methyl)isophthalamide: ¹H NMR (300 MHz, CDCl₃) δ 7.78 (m, 1H), 7.68-7.70 (m, 1H), 7.53-7.57 (m, 1H), 7.36-7.42 (m, 1H), 7.119-7.30 (m, 5H), 6.90 (s, 1H), 6.45 (s, 1H), 5.18 (s, 2H), 4.97 (m, 2H), 4.48 (m, 1H), 3.73-3.75 (m, 3H), 3.00-3.12 (m, 5H), 2.73-2.85 (m, 3H), 2.46 (s, 3H), 1.22 (m, 6H).

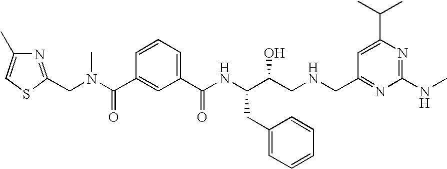

N1-((2S,3R)-3-hydroxy-4-((6-isopropyl-2-(methylamino)pyrimidin-4-yl)methylamino)-1-phenylbutan-2-yl)-N3-methyl-N3-((4-methylthiazol-2-yl)methyl)isophthalamide: ¹H NMR (300 MHz, CDCl₃) δ 7.78 (m, 1H), 7.68-7.71 (m, 1H), 7.55-7.57 (m, 1H), 7.37-7.42 (m, 1H), 7.17-7.30 (m, 5H), 6.91 (s, 1H), 6.36 (s, 1H), 4.98 (m, 2H), 4.48 (m, 1H), 3.69-3.75 (m, 3H), 2.98-3.14 (m, 8H), 2.73-2.88 (m, 3H), 2.47 (s, 3H), 1.22 (m, 6H).

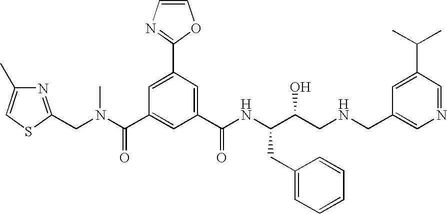

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-2-yl)isophthalamide: ¹H NMR (300 MHz, CDCl₃) δ 8.36-8.41 (m, 3H), 8.24 (s, 1H), 7.84 (s, 1H), 7.77 (s, 1H), 7.55 (m, 1H), 7.24-7.33 (m, 6H), 6.93 (s, 1H), 5.01 (m, 2H), 4.44 (m, 1H), 3.76-3.93 (m, 3H), 3.03-3.17 (m, 5H), 2.88-2.98 (m, 1H), 2.88-2.98 (m, 1H), 2.84-2.86 (m, 2H), 2.49 (s, 3H), 1.25 (m, 6H).

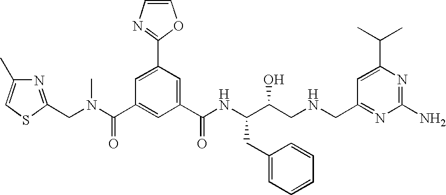

N1-((2S,3R)-4-((2-amino-6-isopropylpyrimidin-4-yl)methylamino)-3-hydroxy-1-phenylbutan-2-yl)-N3-methyl-N3-((4-methylthiazol-2-yl)methyl)-5-(oxazol-2-yl)isophthalamide: ¹H NMR (300 MHz, CDCl₃) δ 8.37 (s, 1H), 8.21 (s, 1H0, 7.86 (s, 1H0, 7.74 (s, 1H), 7.18-7.31 (m, 6H), 6.93 (s, 1H0, 6.46 (s, 1H0, 4.99 (m, 2H), 4.51 (m, 2H), 3.78 (m, 3H), 3.04-3.16 (m, 5H), 2.88 (m, 2H), 2.72-2.81 (m, 1H), 2.48 (s, 3H), 1.20 (m, 6H).

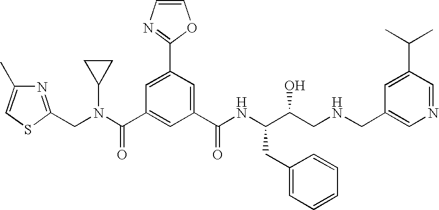

N1-cyclopropyl-N3-((2S,3R)-3-hydroxy-4-((5-isopropylpyridin-3-yl)methylamino)-1-phenylbutan-2-yl)-N1-((4-methylthiazol-2-yl)methyl)-5-(oxazol-2-yl)isophthalamide: ¹H NMR (300 MHz, CDCl₃) δ 8.36-8.41 (m, 3H), 7.94 (s, 1H), 7.78 (m, 1H), 7.16-7.30 (m, 7H), 6.90 (s, 1H), 5.06 (s, 2H), 4.44 (m, 1H), 3.76-3.98 (m, 3H), 3.04 (m, 2H), 2.94 (m, 1H0, 2.85 (m, 2H), 2.49 (s, 3H), 1.36 (m, 6H), 0.49-0.68 (m, 4H).

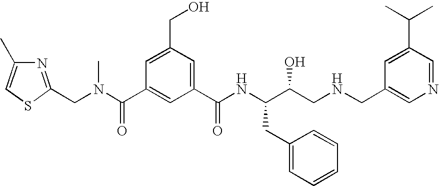

N1-((2S,3R)-3-hydroxy-4-((5-isopropylpyridin-3-yl)methylamino)-1-phenylbutan-2-yl)-5-(hydroxymethyl)-N3-methyl-N3-((4-methylthiazol-2-yl)methyl)isophthalamide: ¹H NMR (300 MHz, CDCl₃) δ 8.41 (m, 2H), 7.68-7.71 (m, 2H0, 7.55-7.59 (m, 2H0, 7.20-7.32 (m, 5H), 6.92 (m, 1H), 4.99 (m, 2H), 4.57 (s, 2H), 4.41 (m, 1H), 3.80-3.91 (m, 2H), 3.75 (m, 1H), 2.90-3.15 (m, 6H), 2.84 (m, 2H), 2.48 (s, 3H), 1.25 (m, 6H).

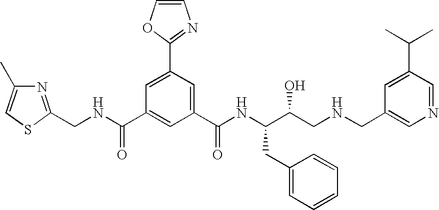

N1-((2S,3R)-3-hydroxy-4-((5-isopropylpyridin-3-yl)methylamino)-1-phenylbutan-2-yl)-N3-((4-methylthiazol-2-yl)methyl)-5-(oxazol-2-yl)isophthalamide: ¹H NMR (300 MHz, CDCl₃) δ 9.09 (m, 1H), 8.52-8.55 (m, 2H), 8.44 (s, 1H), 8.26-8.29 (m, 2H, 7.65 (m, 2H), 7.11-7.24 (m, 5H), 6.76 (s, 1H), 4.80-4.89 (m, 2H), 4.43 (m, 1H), 4.00-4.20 (m, 3H), 2.96-3.12 (m, 4H), 2.80 (m, 1H), 2.30 (s, 3H), 1.13 (m, 6H).

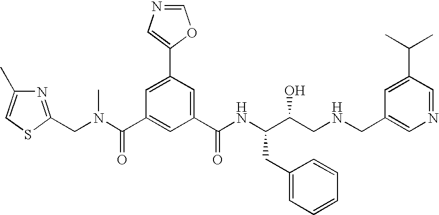

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-5-yl)isophthalamide: ¹H NMR (300 MHz, CDCl₃) δ 8.34-8.37 (m, 2H), 7.89-7.91 (m, 2H), 7.73 (s, 1H), 7.64 (s, 1H), 7.52 (s, 1H), 7.36 (s, 1H), 7.16-7.26 (m, 5H), 6.90 (s, 1H), 4.98 (m, 2H), 4.43 (m, 1H), 3.77-3.89 (m, 3H), 2.83-3.15 (m, 8H), 2.46 (s, 3H), 1.24 (m, 6H).

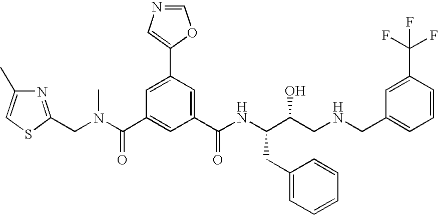

N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-5-yl)isophthalamide: ¹H NMR (300 MHz, CDCl₃) δ 7.85-7.89 (m, 2H0, 7.59-7.68 (m, 3H), 7.41-7.53 (m, 3H), 7.34 (s, 1H), 7.17-7.25 (m, 5H), 6.89 (s, 1H), 4.97 (m, 2H), 4.42 (m, 1H), 3.79-3.89 (m, 3H), 3.00-3.15 (m, 5H), 2.82-2.84 (m, 2H), 2.45 (s, 3H).

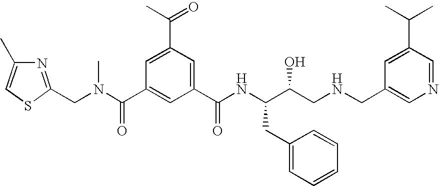

5-acetyl-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.41 (m, 2H), 8.28 (m, 1H), 8.17 (s, 1H), 7.98 (s, 1H), 7.55 (m, 1H), 7.22-7.31 (m, 5H), 6.94 (s, 1H), 5.01 (m, 2H), 4.46 (m, 1H), 3.81-3.93 (m, 2H), 3.75 (m, 1H), 3.02-3.18 (m, 5H), 2.95 (m, 1H), 2.84 (m, 2H), 2.66 (s, 3H), 2.49 (s, 3H), 1.28 (m, 6H).

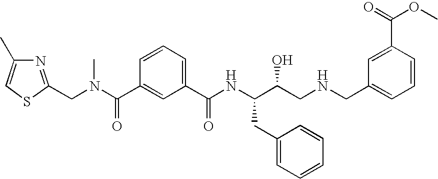

methyl 3-(((2R,3S)-2-hydroxy-3-(3-(methyl((4-methylthiazol-2-yl)methyl)carbamoyl)benzamido)-4-phenylbutylamino)methyl)benzoate: ¹H NMR (300 MHz, CDCl₃+CD₃OD), d: 7.993-7.921 (m, 2H), 7.732-7.648 (m, 2H), 7.429-7.354 (m, 2H), 7.264-7.141 (m, 5H), 6.895 (s, 1H), 4.954 (br, 1.4H), 4.651 (br, 0.6H), 4.378 (m, 1H), 3.903 (s, 3H), 3.847-3.723 (m, 3H), 3.110-2.922 (m, 5H), 2.809 (m 2H), 2.444 (s, 3H).

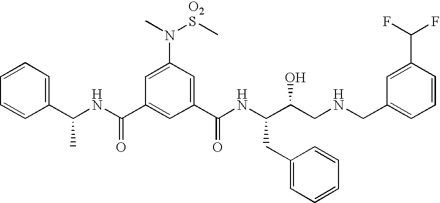

N1-((2S,3R)-4-(3-(difluoromethyl)benzylamino)-3-hydroxy-1-phenylbutan-2-yl)-5-(N-methylmethylsulfonamido)-N3-((R)-1-phenylethyl)isophthalamide: ¹H NMR (300 MHz, CDCl₃+CD₃OD), d: 8.066 (s, 1H), 7.923 (m, 1H), 7.786 (m 1H), 7.479-7.142 (m, 14H), 6.622 (t, J=56.4 Hz, 1H), 5.300 (m, 1H), 4.375 (m, 1H), 3.926-3.734 (m, 3H), 3.487 (s, 3 H), 2.996-2.790 (m, 7H), 1.598 (d, J=7.2 Hz, 3H).

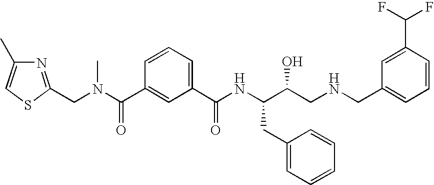

N1-((2S,3R)-4-(3-(difluoromethyl)benzylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.782-7.529 (m, 4H), 7.471-7.368 (m, 5H), 7.272-7.139 (m, 5H), 6.901 (s, 1H), 6.630 (t, J=56.4 Hz, 1H), 4.961 (br, 1.4H), 4.654 (br, 0.6H), 4.376 (m, 1H), 3.891-3.720 (m, 3H), 3.115-2.896 (m, 5H), 2.798 (m, 2H), 2.450 (s, 3H).

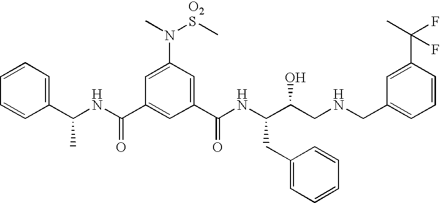

N1-((2S,3R)-4-(3-(1,1-difluoroethyl)benzylamino)-3-hydroxy-1-phenylbutan-2-yl)-5-(N-methylmethylsulfonamido)-N3-((R)-1-phenylethyl)isophthalamide: ¹H NMR (300 MHz, CDCl₃+CD₃OD), d: 8.080 (m, 1H), 7.923 (m, 1H), 7.792 (m 1H), 7.479-7.157 (m, 14H), 5.304 (m, 1H), 4.371 (m, 1H), 3.972-3.739 (m, 3H), 3.281 (s, 3H), 2.917 (m, 2H), 2.822 (m, 5H), 1.895 (t, J=18.3 Hz, 3H), 1.602 (d, J=6.6 Hz, 3H).

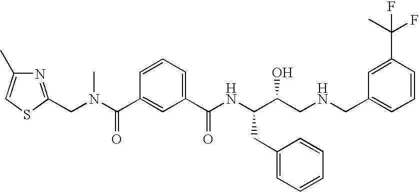

N1-((2S,3R)-4-(3-(1,1-difluoroethyl)benzylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.736-7.656 (m, 2H), 7.471-7.372 (m, 6H), 7.276-7.152 (m, 5H), 6.899 (s, 1H), 4.962 (br, 1.4H), 4.659 (br, 0.6H), 4.385 (m, 1H), 3.890-3.725 (m, 3H), 3.116-2.901 (m, 5H), 2.817 (m, 2H), 2.450 (s, 3H), 1.909 (t, J=18.2 Hz, 3H).

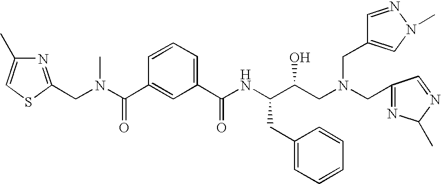

N1-((2S,3R)-4-(bis((1-methyl-1H-pyrazol-4-yl)methyl)amino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.713 (m, 2H), 7.588-7.435 (m, 2H), 7.385 (s, 2H), 7.298-7.166 (m, 7H), 6.925 (s, 1H), 4.988 (br, 1.4H), 4.683 (br, 0.6H), 4.365 (m, 1H), 3.925-3.877 (m, 3H), 3.641-3.441 (m, 5H), 3.134-2.942 (m, 5H), 2.556 (m, 2H), 2.469 (s, 3H).

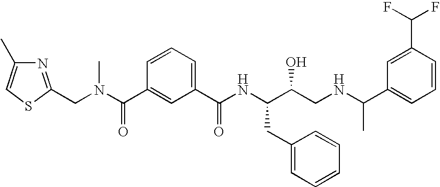

N1-((2S,3R)-4-(1-(3-(difluoromethyl)phenyl)ethylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.766-7.550 (m, 3H), 7.463-7.147 (m, 10H), 6.922 (m, 1H), 6.641 (m, 1H), 4.968 (br, 1.4H), 4.664 (br, 0.6H), 4.359 (m, 1H), 3.800 (m, 1H), 3.662 (m, 1H), 3.125-2.858 (m, 5H), 2.724-2.557 (m, 2H), 2.457 (s, 3H), 1.431 (d, J=6.6 Hz, 3H).

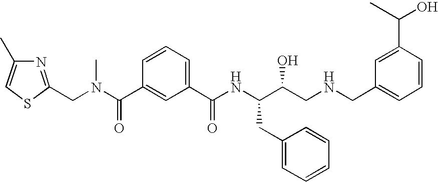

N1-((2S,3R)-3-hydroxy-4-(3-(1-hydroxyethyl)benzylamino)-1-phenylbutan-2-yl)-N3-methyl-N3-((4-methylthiazol-2-yl)methyl)isophthalamide: ¹H NMR (300 MHz, CDCl₃+CD₃OD), d: 7.693-7.160 (m, 13H), 6.915 (m, 1H), 4.955 (br, 1.4H), 4.813 (m, 1H), 4.638 (br, 0.6H), 4.326 (m, 1H), 3.854-3.722 (m, 3H), 3.110-2.878 (m, 5H), 2.793 (m, 2 H), 2.446 (s, 3H), 1.450 (d, J=6.6 Hz, 3H).

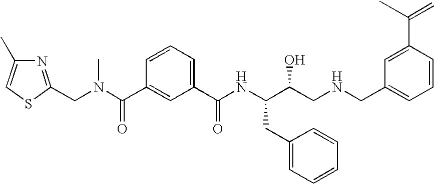

N1-((2S,3R)-3-hydroxy-1-phenyl-4-(3-(prop-1-en-2-yl)benzylamino)butan-2-yl)-N3-methyl-N3-((4-methylthiazol-2-yl)methyl)isophthalamide: ¹H NMR (300 MHz, CDCl₃+CD₃OD), d: 7.757-7.526 (m, 3H), 7.420-7.149 (m, 10H), 6.891 (s, 1H), 5.370 (s, 1 H), 5.085 (s, 1H), 4.958 (br, 1.4H), 4.653 (br, 0.6H), 4.395 (m, 1H), 3.868-3.740 (m, 3H), 3.111-2.950 (m, 5H), 2.837 (m, 2H), 2.450 (s, 3H), 2.140 (s, 3H).

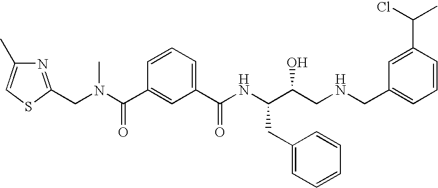

N1-((2S,3R)-4-(3-(1-chloroethyl)benzylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 7.856-7.553 (m, 3H), 7.444-7.186 (m, 10H), 6.909 (s, 1H), 5.750 (m, 0.3H), 5.091 (m, 0.7H), 4.969 (br, 1.4H), 4.664 (br, 0.6H), 4.374 (m, 1H), 3.884-3.675 (m, 3H), 3.120-2.921 (m, 5H), 2.834 (m, 2H), 2.456 (s, 3H), 1.845 (m, 3H).

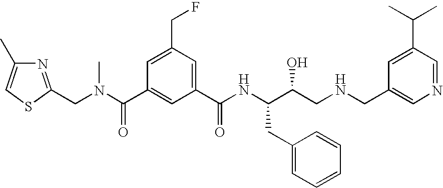

5-(fluoromethyl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃+CD₃OD), d: 8.350 (s, 2H), 7.716 (m, 2H), 7.557 (m, 1H), 7.238 (m, 5H), 6.923 (s, 1H), 5.488 (s, 1H), 5.330 (s, 1H), 4.973 (br, 1.4H), 4.664 (br, 0.6H), 4.355 (m, 1H), 3.861-3.734 (m, 3H), 3.432 (m, 1H), 3.128-2.892 (m, 5H), 2.797 (m, 2H), 2.464 (s, 3H), 1.263 (d, J=7.2 Hz, 3H).

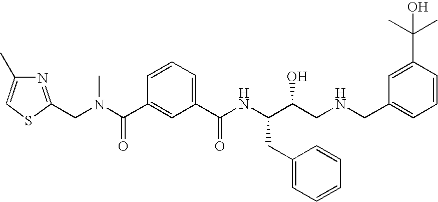

N1-((2S,3R)-3-hydroxy-4-(3-(2-hydroxypropan-2-yl)benzylamino)-1-phenylbutan-2-yl)-N3-methyl-N3-((4-methylthiazol-2-yl)methyl)isophthalamide: ¹H NMR (300 MHz, CDCl₃+CD₃OD), d: 7.694-7.158 (m, 13H), 6.915 (s, 1H), 4.956 (br, 1.4H), 4.649 (br, 0.6 H), 4.298 (m, 1H), 3.900-3.770 (m, 3H), 3.100-2.941 (m, 5H), 2.838 (m, 2H), 2.447 (s, 3 H), 1.521 (s, 6H).

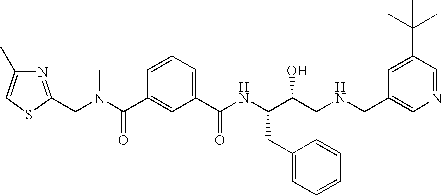

N1-((2S,3R)-4-((5-tert-butylpyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 8.509 (d, J=2.4 Hz, 1H), 8.330 (d, J=1.8 Hz, 1H), 7.682 (m, 3H), 7.528 (m, 1H), 7.413 (m, 1H), 7.262-7.138 (m, 5H), 6.903 (s, 1H), 4.961 (br, 1.4H), 4.657 (br, 0.6H), 4.349 (m, 1H), 3.786 (m, 3H), 3.111-2.920 (m, 5H), 2.797 (m, 2H), 2.443 (s, 3 H), 1.317 (s, 9H).

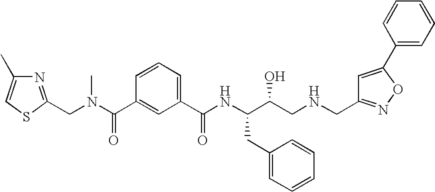

N1-((2S,3R)-3-hydroxy-1-phenyl-4-((5-phenylisoxazol-3-yl)methylamino)butan-2-yl)-N3-methyl-N3-((4-methylthiazol-2-yl)methyl)isophthalamide: ¹H NMR (300 MHz, CDCl₃+CD₃OD), d: 7.714 (m, 4H), 7.435 (m, 4H), 7.315 (m, 1H), 7.222 (m, 5H), 6.889 (s, 1H), 6.557 (s, 1H), 4.939 (br, 1.4H), 4.607 (br, 0.6H), 4.399 (m, 1H), 3.926 (m, 2H), 3.770 (m, 1H), 3.097-2.916 (m, 5H), 2.849 (m, 2H), 2.439 (s, 3H).

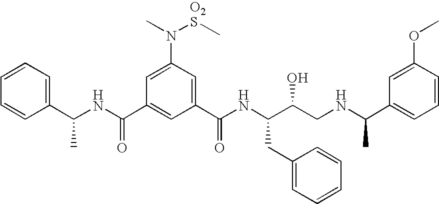

N1-((2S,3R)-3-hydroxy-4-((R)-1-(3-methoxyphenyl)ethylamino)-1-phenylbutan-2-yl)-5-(N-methylmethylsulfonamido)-N3-((R)-1-phenylethyl)isophthalamide: ¹H NMR (300 MHz, CDCl₃+CD₃OD), d: 8.071 (m, 1H), 7.956 (m, 1H), 7.812 (m 1H), 7.418-7.159 (m, 11 H), 6.905-6.769 (m, 3H), 5.339 (m, 1H), 4.373 (m, 1H), 3.765 (s, 3H), 3.698 (m, 2H), 3.337 (s, 3H), 2.873 (m, 5H), 2.736-2.579 (m, 2H), 1.643 (d, J=6.6 Hz, 3H), 1.439 (d, J=6.9 Hz, 3H).

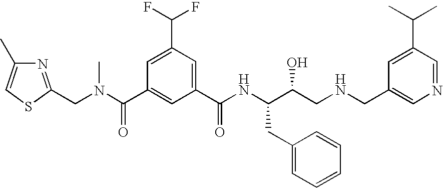

5-(difluoromethyl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃+CD₃OD), d: 8.337 (s, 2H), 7.857 (s, 2H), 7.690 (s, 1H), 7.540 (m, 1H), 7.224 (m, 5H), 6.922 (s, 1H), 6.658 (t, J=56 Hz, 1H), 4.973 (br, 1.4H), 4.636 (br, 0.6H), 4.380 (m, 1H), 3.794 (m, 3H), 3.136-2.879 (m, 6H), 2.797 (m, 2H), 2.457 (s, 3H), 1.259 (d, J=6.9 Hz, 6H).

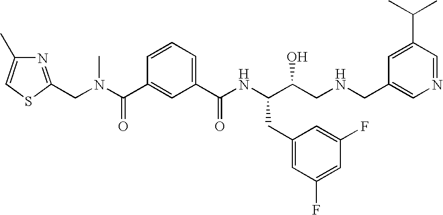

N1-((2S,3R)-1-(3,5-difluorophenyl)-3-hydroxy-4-((5-isopropylpyridin-3-yl)methylamino)butan-2-yl)-N3-methyl-N3-((4-methylthiazol-2-yl)methyl)isophthalamide: ¹H NMR (300 MHz, CDCl₃+CD₃OD), d: 8.339 (s, 2H), 7.741 (m, 2H), 7.537 (m, 1H), 7.418 (m, 1H), 6.898 (s, 1H), 6.788 (m, 2H), 6.608 (m, 1H), 4.960 (br, 1.4H), 4.670 (br, 0.6H), 4.323 (m, 1H), 3.847-3.718 (m, 3H), 3.108-2.849 (m, 6H), 2.792 (m, 2H), 2.442 (s, 3H), 1.255 (d, J=6.9 Hz, 6H).

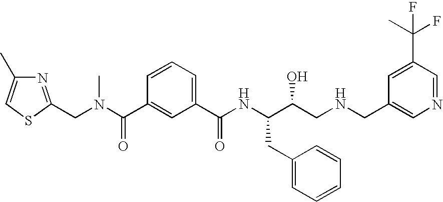

N1-((2S,3R)-4-((5-(1,1-difluoroethyl)pyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 8.638 (d, J=10.2 Hz, 2H), 7.847 (s, 1H), 7.700 (m, 2H), 7.550 (br, 1H), 7.423 (m, 1H), 7.233 (m, 5H), 6.913 (s, 1H), 4.958 (br, 1.4H), 4.661 (br, 0.6H), 4.350 (m, 1H), 3.856 (s, 2H), 3.777 (m, 1H), 3.113-2.897 (m, 5H), 2.787 (m, 2H), 2.449 (s, 3H), 1.946 (ddd, J=20.0, 2.4 Hz, 3H).

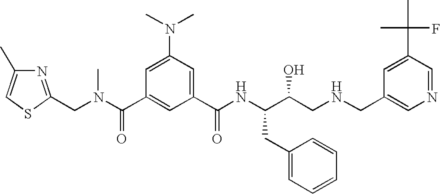

5-(dimethylamino)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 8.491 (d, J=13.8 Hz, 2H), 7.738 (m, 1H), 7.215 (m, 5H), 7.019 (s, 1H), 6.903 (m, 1H), 4.954 (br, 1.2H), 4.667 (br, 0.8H), 4.316 (m, 1H), 3.821 (s, 2H), 3.761 (m, 1H), 3.082-2.927 (m, 11H), 2.773 (m, 2H), 2.440 (s, 3H), 1.718 (d, J=21.9 Hz, 6H).

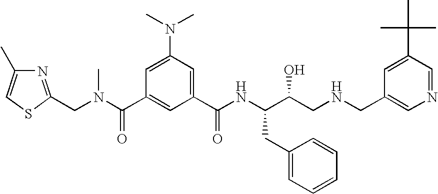

N1-((2S,3R)-4-((5-tert-butylpyridin-3-yl)methylamino)-3-hydroxy-1-phenylbutan-2-yl)-5-(dimethylamino)-N3-methyl-N3-((4-methylthiazol-2-yl)methyl)isophthalamide: ¹H NMR (300 MHz, CDCl₃+CD₃OD), d: 8.485 (d, J=2.4 Hz, 1H), 8.309 (d, J=2.1 Hz, 1H), 7.684 (m, 1H), 7.190 (m, 5H), 7.013 (m, 1H), 6.874 (br, 3H), 4.932 (br, 1.2H), 4.651 (br, 0.8H), 4.319 (m, 1H), 3.770 (m, 3H), 3.076-2.928 (m, 11H), 2.773 (m, 2H), 2.421 (s, 3H), 1.296 (s, 9H).

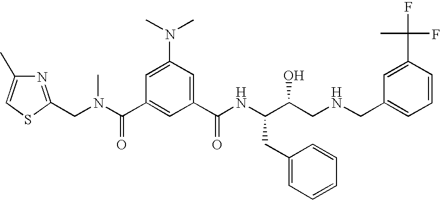

N1-((2S,3R)-4-(3-(1,1-difluoroethyl)benzylamino)-3-hydroxy-1-phenylbutan-2-yl)-5-(dimethylamino)-N3-methyl-N3-((4-methylthiazol-2-yl)methyl)isophthalamide: ¹H NMR (300 MHz, CDCl₃+CD₃OD), d: 7.484 (s, 1H), 7.401 (m, 3H), 7.235 (m, 5H), 7.035 (s, 1H), 6.898 (m, 3H), 4.973 (br, 1.2H), 4.677 (br, 0.8H), 4.358 (m, 1H), 3.858 (m, 2H), 3.748 (m, 1H), 3.117-2.968 (m, 11H), 2.803 (m, 2H), 2.459 (s, 3H), 1.917 (t, J=18.1 Hz, 3H).

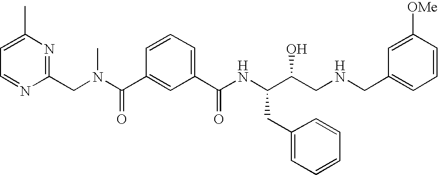

N1-((2S,3R)-3-hydroxy-4-(3-methoxybenzylamino)-1-phenylbutan-2-yl)-N3-methyl-N3-((4-methylpyrimidin-2-yl)methyl)isophthalamide: ¹H NMR (300 MHz, CDCl₃): 2.43-2.46 (m, 3H), 2.70-2.75 (m, 2H), 2.89-3.02 (m, 5H), 3.57-3.77 (m, 6H), 4.22-4.38 (m, 1H), 4.50 (s, 1H), 4.87 (s, 1H), 6.72-6.75 (m, 2H), 6.81-6.84 (m, 2H), 6.98 (d, 1H, J=4.8 Hz), 7.11-7.25 (m, 6H), 7.53-7.58 (m, 2H), 7.76-7.80 (m, 1H), 8.44-8.50 (m, 1H).

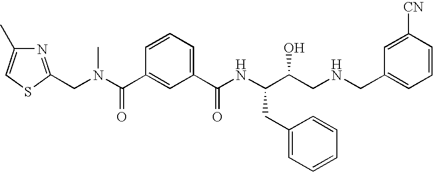

N1-((2S,3R)-4-(3-cyanobenzylamino)-3-hydroxy-1-phenylbutan-2-yl)-N3-methyl-N3-((4-methylthiazol-2-yl)methyl)isophthalamide: ¹H NMR (300 MHz, CDCl₃): 2.47 (s, 3H), 2.72-2.84 (m, 2H), 2.97-3.14 (m, 5H), 3.68-3.72 (m, 1H0, 3.80-3.90 (m, 2H), 4.35-4.41 (m, 1H), 4.66 (br s, 1H), 4.98 (br s, 1H), 6.91 (s, 1H), 7.19-7.32 (m, 6H), 7.40-7.47 (m, 2H), 7.55-7.68 (m, 5H).

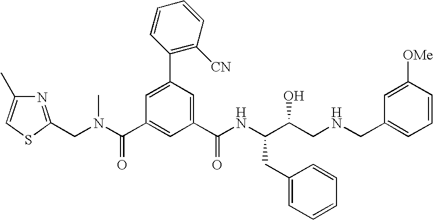

2′-cyano-N3-((2S,3R)-3-hydroxy-4-(3-methoxybenzylamino)-1-phenylbutan-2-yl)-N5-methyl-N5-((4-methylthiazol-2-yl)methyl)biphenyl-3,5-dicarboxamide: ¹H NMR (300 MHz, CDCl₃): 2.47 (s, 3H), 2.81-2.83 (m, 2H), 2.92-2.99 (m, 2H), 3.13 (s, 3H), 3.68-3.86 (m, 6H), 4.41-4.46 (m, 1H), 4.74 (s, 1H), 4.99 (s, 1H), 6.75-6.91 (m, 3H), 7.15-7.25 (m, 7H), 7.49-7.54 (m, 2H), 7.66-7.71 (m, 1H), 7.76-7.81 (m, 2H), 7.88-7.91 (m, 2H).

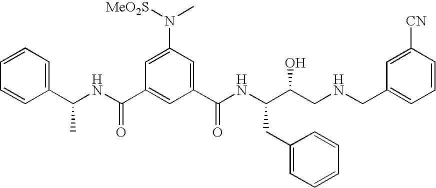

N1-((2S,3R)-4-(3-cyanobenzylamino)-3-hydroxy-1-phenylbutan-2-yl)-5-(N-methylmethylsulfonamido)-N3-((R)-1-phenylethyl)isophthalamide: ¹H NMR (300 MHz, CDCl₃): 1.63 (d, 3H, J=6.9 Hz), 2.73-2.83 (m, 5H), 2.96-2.99 (m, 2H), 3.32 (s, 3H), 3.68 (s, 1H), 3.72-3.97 (m, 2H), 4.36-4.41 (m, 1H), 5.29-5.34 (m, 1H), 6.95 (d, 1H, J=7.8 Hz), 7.21-7.47 (m, 10H), 7.54-7.59 (m, 2H), 7.79 (m, 1H), 7.94 (m, 1H), 8.02 (m, 1H).

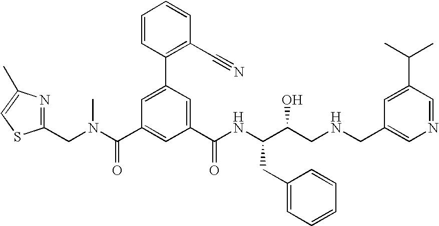

2′-cyano-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide: ¹H NMR (300 MHz, CDCl₃): 1.22-1.24 (m, 6H), 2.47 (s, 3H), 2.81-3.14 (m, 8H), 3.72-3.89 (m, 3H), 4.40-4.45 (m, 1H), 4.79 (br s, 1H), 5.00 (br s, 1H), 6.90 (s, 1H), 7.18-7.30 (m, 7H0, 7.51-7.56 (m, 2H), 7.68-7.90 (m, 4H), 8.38 (t, 2H, J=2.7 Hz).

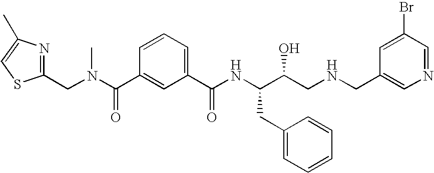

N1-((2S,3R)-4-((5-bromopyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃): 2.47 (s, 3H), 2.72-2.83 (m, 2H), 2.97-3.12 (m, 5H), 3.68-3.86 (m, 3H), 4.33-4.41 (m, 1H), 4.66 (br s, 1H), 4.98 (br s, 1H), 6.91 (s, 1H), 7.21-7.30 (m, 6H), 7.41 (t, 1H, J=7.5 Hz), 7.64 (s, 1H), 7.66 (s, 1H), 7.86 ((t, 1H, J=2.1 Hz), 8.47 (br s, 1H), 8.57 (d, 1H, J=1.6 Hz).

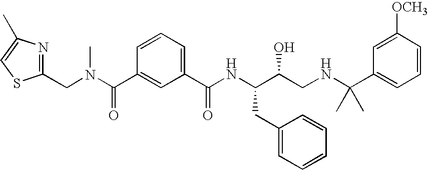

N1-((2S,3R)-3-hydroxy-4-(2-(3-methoxyphenyl)propan-2-ylamino)-1-phenylbutan-2-yl)-N3-methyl-N3-((4-methylthiazol-2-yl)methyl)isophthalamide: ¹H NMR (300 MHz, CDCl₃): 1.52 (d, 6H, J=7.2 Hz), 2.47-2.53 (m, 5H), 2.97-3.14 (m, 5H), 3.55-3.63 (m, 1H), 4.38-4.44 (m, 1H), 4.63 (br s, 1H), 4.98 (br s, 1H), 6.76-6.79 (m, 1H), 6.91 (s, 1H), 7.02-7.04 (m, 2H), 7.18-7.30 (m, 7H), 7.42 (t, 1H, J=7.8 Hz), 7.72-7.74 (m, 2H).

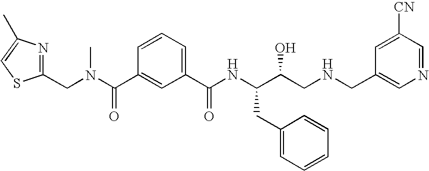

N1-((2S,3R)-4-((5-cyanopyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃): 2.47 (s, 3H), 2.71-2.80 (m, 2H), 3.02-3.15 (m, 5H), 3.71-3.74 (m, 1H), 3.89 (s, 2H), 4.34-4.39 (m, 1H), 4.70 (br s, 1H), 4.98 (br s, 1H), 6.92 (s, 1H), 7.24-7.30 (m, 6H), 7.43 (t, 1H, J=7.5 Hz), 7.66-7.70 (m, 2H), 8.01-8.02 (t, 1H, J=2.1 Hz), 8.78 (t, 1H, J=2.4 Hz).

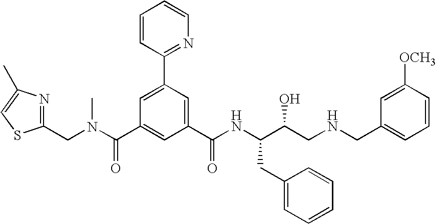

N1-((2S,3R)-3-hydroxy-4-(3-methoxybenzylamino)-1-phenylbutan-2-yl)-N3-methyl-N3-((4-methylthiazol-2-yl)methyl)-5-(pyridin-2-yl)isophthalamide: ¹H NMR (300 MHz, CDCl₃): 2.49 (s, 1H), 2.83-2.86 (m, 2H), 3.04-3.18 (m, 5H), 3.78-3.91 (m, 6H), 4.40-4.46 (m, 1H), 4.72 (br s, 1H), 5.01 (br s, 1H), 6.78-6.81 (m, 1H), 6.92-6.95 (m, 2H), 7.20-7.33 (m, 8H), 7.74-7.81 (m, 2H), 8.21 (s, 1H), 8.34 (s, 1H), 8.70 (d, 1H, J=4.8 Hz).

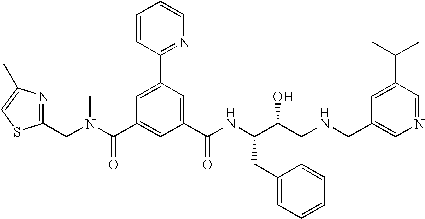

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-2-yl)isophthalamide: ¹H NMR (300 MHz, CDCl₃): 1.24 (d, 6H, J=6.9 Hz), 2.49 (s, 3H), 2.84-2.95 (m, 3H), 3.04-3.18 (m, 5H0, 3.79-3.87 (m, 3H), 4.39-4.46 (m, 1H), 4.71 (br s, 1H), 5.01 (br s, 1H), 6.93 (s, 1H), 7.16-7.32 (m, 8H), 7.57 (br s, 1H), 7.70-7.79 (m, 2H), 8.28-8.40 (m, 3H), 8.67 (d, 1H, 4.5 Hz).

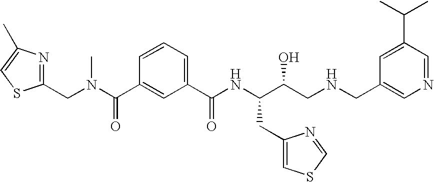

N1-((2S,3R)-3-hydroxy-4-((5-isopropylpyridin-3-yl)methylamino)-1-(thiazol-4-yl)butan-2-yl)-N3-methyl-N3-((4-methylthiazol-2-yl)methyl)isophthalamide: ¹H NMR (300 MHz, CDCl₃): 1.27 (d, 6H, J=6.9 Hz), 2.49 (s, 3H), 2.65-2.71 (m, 1H), 2.82-2.98 (m, 2H), 3.20-3.20 (m, 4H), 3.33-3.38 (m, 1H), 3.45-3.52 (m, 2H), 3.81-3.86 (m, 2H), 4.40-4.44 (m, 1H), 4.71 (br s, 1H), 5.03 (br s, 1H), 6.93 (s, 1H), 7.22-7.23 (br s, 1H), 7.52-7.57 (m, 2H), 7.91-7.99 (m, 3H), 8.40 (d, 1H, J=1.8 Hz), 8.82 (s, 1H).

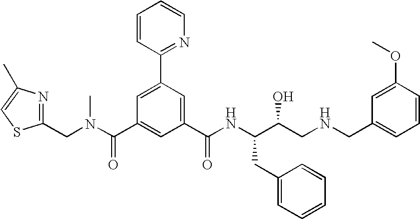

N1-((2S,3R)-3-hydroxy-4-(3-methoxybenzylamino)-1-phenylbutan-2-yl)-N3-methyl-N3-((4-methylthiazol-2-yl)methyl)-5-(pyridin-3-yl)isophthalamide: ¹H NMR (300 MHz, CDCl₃): 2.48 (s, 3H), 2.86 (s, 3H), 3.02-3.18 (m, 4H), 3.78-3.89 (m, 6H0, 4.41-4.51 (m, 1H), 4.71 (br s, 1H), 5.01 (br s, 1H), 6.78-6.81 (m, 1H), 7.19-7.41 (m, 8H), 7.73-7.91 (m, 3H0, 8.63-8.65 (m, 1H), 8.81 (br s, 1H).

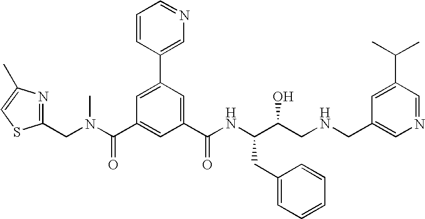

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-3-yl)isophthalamide: ¹H NMR (300 MHz, CDCl₃): 1.22 (d, 6H, J=6.9 Hz), 2.47 (s, 3H), 2.86-3.16 (m, 8H), 4.02-4.09 (m, 2H), 4.41-4.49 (m, 1H), 4.67 (s, 1H), 4.99 (s, 1H), 6.92 (s, 1H), 7.14-7.32 (m, 7H), 7.72-7.86 (m, 4H), 8.07 (s, 1H), 8.36 (s, 1H), 8.45 (br s, 1H).

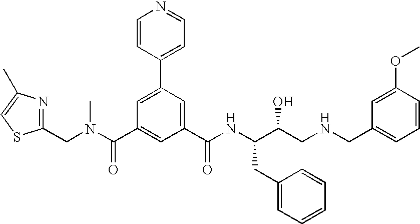

N1-((2S,3R)-3-hydroxy-4-(3-methoxybenzylamino)-1-phenylbutan-2-yl)-N3-methyl-N3-((4-methylthiazol-2-yl)methyl)-5-(pyridin-4-yl)isophthalamide: ¹H NMR (300 MHz, CDCl₃): 2.47 (s, 3H), 2.85 (br s, 2H)<3.03-3.16 (m, 6H), 3.76-3.87 (m, 5H), 4.42-4.48 (m, 1H), 4.65 (br s, 1H), 4.99 (br s, 1H), 6.76-6.79 (m, 1H), 6.88-6.91 (m, 2H), 7.18-7.30 (m, 5H), 7.40 (d, 2H, J=5.1 Hz), 7.72-7.76 (m, 2H), 7.89-7.92 (m, 2H), 8.62-8.64 (d, 2H, J=5.1 Hz).

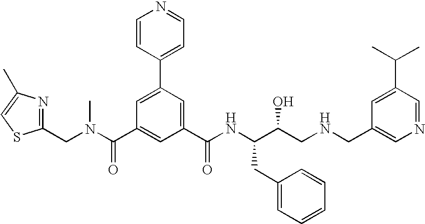

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-4-yl)isophthalamide: ¹H NMR (300 MHz, CDCl₃): 1.24 (d, 6H, J=6.6 Hz), 2.47 (s, 3H), 2.83-3.17 (m, 8H), 3.78-3.88 (m, 3H), 4.40-4.47 (m, 1H), 4.67 (br s, 1H), 4.99 (br s, 1H), 6.91 (s, 1H), 7.18-7.30 (m, 5H), 7.41 (d, 2H, J=5.7 Hz), 7.51 (br s, 1H), 7.75 (s, 1H), 7.91 (br s, 2H), 8.35-8.37 (m, 2H), 8.65 (d, 2H, J=6.8 Hz).

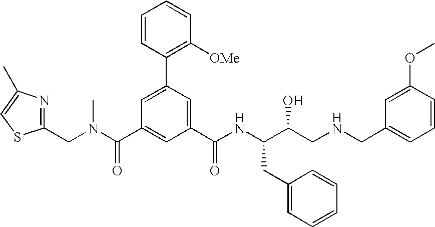

N3-((2S,3R)-3-hydroxy-4-(3-methoxybenzylamino)-1-phenylbutan-2-yl)-2′-methoxy-N5-methyl-N5-((4-methylthiazol-2-yl)methyl)biphenyl-3,5-dicarboxamide: ¹H NMR (300 MHz, CDCl₃): 2.48 (s, 3H), 2.80 (d, 2H, J=4.8 Hz), 3.01-3.09 (m, 5H), 3.68-3.86 (m, 8H), 4.38-4.46 (m, 1H), 4.78 (br s, 1H), 5.00 (br s, 1H), 6.78-6.81 (m, 1H), 6.90-6.93 (m, 3H), 7.02-7.09 (m, 2H), 7.26-7.39 (m, 8H), 7.72-7.77 (m, 2H), 7.87 (s, 1H).

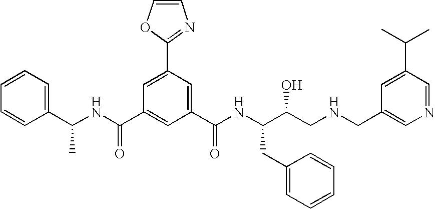

N1-((2S,3R)-3-hydroxy-4-((5-isopropylpyridin-3-yl)methylamino)-1-phenylbutan-2-yl)-5-(oxazol-2-yl)-N3-((R)-1-phenylethyl)isophthalamide: ¹H NMR (300 MHz, CDCl₃): 1.19 (d, 6H, 6.9 Hz), 1.64 (d, 3H, J=7.2 Hz), 2.76-3.04 (m, 5H), 3.71-3.97 (m, 3H), 4.41-4.48 (m, 1H), 5.33-5.41 (m, 1H), 7.17-7.48 (m, 11H), 7.63-7.66 (m, 2H), 8.17 (t, 1H, J=1.5 Hz), 8.27 (t, 1H, J=2.1 hz)<8.40 (d, 1H, J=1.8 Hz), 8.44 (s, 1H).

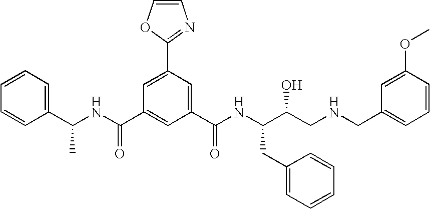

N1-((2S,3R)-3-hydroxy-4-(3-methoxybenzylamino)-1-phenylbutan-2-yl)-5-(oxazol-2-yl)-N3-((R)-1-phenylethyl)isophthalamide: ¹H NMR (300 MHz, CDCl₃): 1.63 (d, 3H, J=6.6 Hz), 2.84-2.97 (m, 4H), 3.74-3.89 (m, 6H), 4.48-4.52 (m, 1H), 5.30-5.37 (m, 1H), 6.75-6.79 (m, 1H), 6.88-6.91 (m, 2H), 7.11-7.43 (m, 12H), 7.64 (s, 1H), 8.13 (s, 1H), 8.29 (s, 1H), 8.39 (s, 1H).

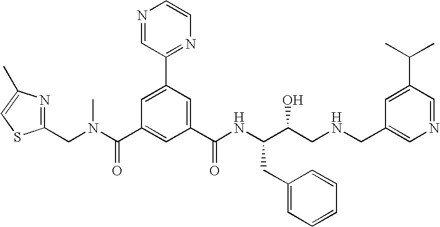

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyrazin-2-yl)isophthalamide: ¹H NMR (300 MHz, CDCl₃): 1.25 (d, 6H, J=6.9 Hz), 2.48 (s, 1H), 2.84-3.18 (m, 8H), 3.80-3.92 (m, 3H), 4.40-4.48 (m, 1H), 4.70 (br s, 1H), 5.01 (br s, 1H), 6.93 (s, 1H), 7.20-7.30 (m, 6H), 7.55 (t, 1H, J=2.1 Hz), 8.37-8.40 (m, 4H), 8.58-8.63 (m, 2H), 9.04 (s, 1H).

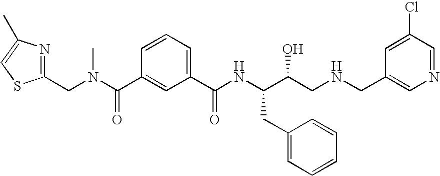

N1-((2S,3R)-4-((5-chloropyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃): 2.47 (s, 3H), 2.72-2.83 (m, 2H), 2.96-3.13 (m, 5H), 3.70-3.88 (m, 3H), 4.34-4.40 (m, 1H), 4.66 (br s, 1H), 4.98 (br s, 1H), 6.91 (s, 1H), 7.21-7.30 (m, 6H), 7.41 (t, 1H, J=7.5 Hz), 7.64-7.72 (m, 3H), 8.43-8.48 (m, 2H).

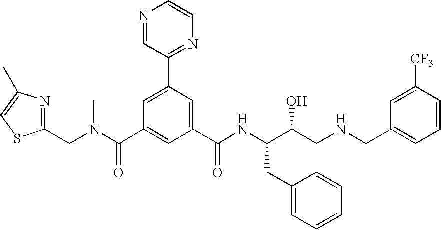

N1-((2S,3R)-3-hydroxy-1-phenyl-4-(3-(trifluoromethyl)benzylamino)butan-2-yl)-N3-methyl-N3-((4-methylthiazol-2-yl)methyl)-5-(pyrazin-2-yl)isophthalamide: ¹H NMR (300 MHz, CDCl₃): 2.48 (s, 3H), 2.84 (s, 3H), 3.04-3.18 (m, 4H), 3.78-3.96 (m, 3H), 4.38-4.49 (m, 1H), 4.68 (br s, 1H), 5.00 (br s, 1H), 6.92 (s, 1H), 7.18-7.30 (m, 4H), 7.40-7.61 (m, 4H), 8.56-8.59 (m, 2H), 9.01 (s, 1H).

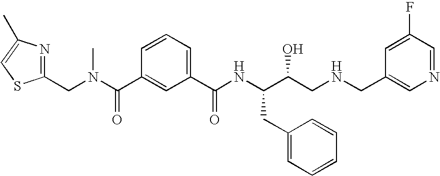

N1-((2S,3R)-4-((5-fluoropyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃): 2.48 (s, 3H), 2.73-2.85 (m, 2H), 2.98-3.14 (m, 5H), 3.48-3.74 (m, 1H), 3.82-3.93 (m, 4H), 4.35-4.44 (m, 1H), 4.67 (br s, 1H), 4.99 (br s, 1H), 6.92 (s, 1H), 7.20-7.33 (m, 7H), 7.41-7.46 (m, 2H), 7.66 (s, 1H), 7.68 (s, 1H), 7.73 (s, 1H), 8.39 (br s, 1H).

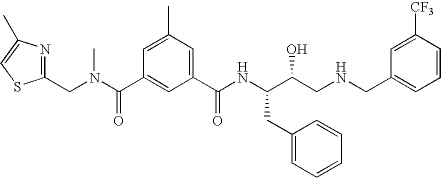

N1-((2S,3R)-3-hydroxy-1-phenyl-4-(3-(trifluoromethyl)benzylamino)butan-2-yl)-N3,5-dimethyl-N3-((4-methylthiazol-2-yl)methyl)isophthalamide: ¹H NMR (300 MHz, CDCl₃): 2.33 (s, 3H), 2.47 (s, 3H), 2.73-2.84 (m, 2H), 2.99-3.12 (m, 5H), 3.64-3.72 (m, 1H), 3.81-3.92 (m, 2H), 4.32-4.43 (m, 1H), 4.64 (br s, 1H), 4.97 (br s, 1H), 6.90 (s, 1H), 7.19-7.30 (m, 7H), 7.45-7.47 (m, 2H), 7.52-7.54 (m, 2H), 7.60 (s, 1H).

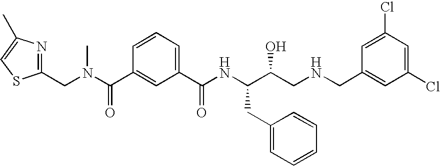

N1-((2S,3R)-4-(3,5-dichlorobenzylamino)-3-hydroxy-1-phenylbutan-2-yl)-N3-methyl-N3-((4-methylthiazol-2-yl)methyl)isophthalamide: ¹H NMR (300 MHz, CDCl₃): 2.47 (s, 3H), 2.70-2.82 (m, 2H), 2.96-3.14 (m, 5H), 3.69-3.83 (m, 5H), 4.37-4.42 (m, 1H), 4.66 (br s, 1H), 4.98 (br s, 1H), 6.91 (s, 1H), 7.19-7.30 (m, 8H), 7.41 (t, 1H, J=7.5 Hz), 7.65-7.68 (m, 2H), 7.74 (s, 1H).

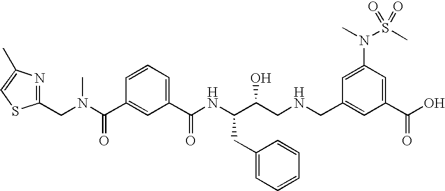

3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoic acid: ¹H NMR (300 MHz, CDCl₃) δ 8.58 (s, 1H), 7.98 (s, 1H), 7.66-7.85 (m, 2H), 7.49-7.66 (m, 2H), 7.39-7.49 (m, 1H), 7.07-7.38 (m, 5H), 6.92 (s, 1H), 6.80-6.83 (m, 1H), 4.87-5.06 (m, 2H), 4.28-4.47 (m, 1H), 4.06-4.23 (m, 2H), 3.92-4.06 (m, 1H), 3.21-3.44 (m, 2H), 3.30 (s, 3H), 3.06-3.21 (m, 3H), 2.99 (s, 3H), 2.82 (s, 3H), 2.50-3.44 (m, 1H), 2.45 (s, 3H).

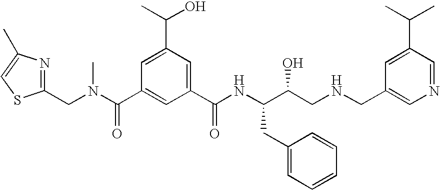

N1-((2S,3R)-3-hydroxy-4-((5-isopropylpyridin-3-yl)methylamino)-1-phenylbutan-2-yl)-5-(1-hydroxyethyl)-N3-methyl-N3-((4-methylthiazol-2-yl)methyl)isophthalamide: ¹H NMR (300 MHz, CDCl₃) d 8.34 (s, 1H), 8.30 (s, 1H), 7.69-7.42 (m, 1H), 7.47-7.74 (m, 3H), 7.13-7.22 (m, 5H), 6.87 (s, 1H), 4.92 (m, 2H), 4.79 (m, 1H), 4.40 (m, 1H), 3.80-3.88 (m, 3H), 2.86-3.10 (m, 7H), 2.44 (s, 3H), 1.34-1.41 (m, 3H), 1.20-1.23 (m, 6H).

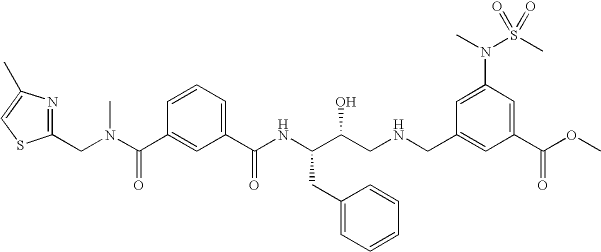

methyl 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoate: ¹H NMR (300 MHz, CDCl₃) d 7.96 (s, 1H), 7.90-7.91 (m, 1H), 7.71-7.83 (m, 1H), 7.65-7.71 (m, 2H), 7.54-7.65 (m, 1H), 7.42-7.47 (m, 1H), 7.17-7.32 (m, 6H), 6.92 (s, 1H), 6.67-6.89 (m, 1H), 5.00 (s, 2H), 4.34-4.46 (m, 1H), 3.95 (s, 3H), 3.90 (s, 2H), 3.64-3.75 (m, 1H), 3.36 (s, 3H), 2.95-3.19 (m, 5H), 2.85 (s, 3H), 2.79-2.81 (m, 2H), 2.48 (s, 3H).

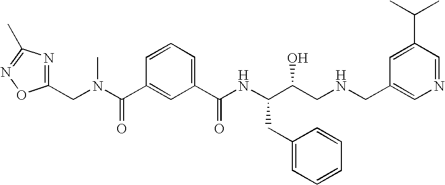

N1-((2S,3R)-3-hydroxy-4-((5-isopropylpyridin-3-yl)methylamino)-1-phenylbutan-2-yl)-N3-methyl-N3-((3-methyl-1,2,4-oxadiazol-5-yl)methyl)isophthalamide: ¹H NMR (300 MHz, CDCl₃+CD₃OD): 1.27 (d, 6H, J=6.9 Hz), 2.45 (s, 3H), 2.82 (d, 2H, J=4.5 Hz), 2.84-3.06 (m, 1H), 3.03 (d, 2H, J=4.5 Hz), 3.08-3.22 (m, 3H), 3.66-3.80 (m, 1H), 3.80-3.84 (m, 2H), 4.36-4.50 (m, 1H), 4.88-5.04 (m, 2H) 7.20-7.34 (m, 6H), 7.40-7.48 (m, 1H), 7.50-7.54 (m, 1H), 7.58-7.64 (m, 1H), 7.66-7.74 (m, 1H), 7.80 (br s, 1H), 8.40 (br s, 1H).

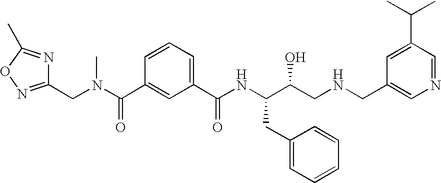

N1-((2S,3R)-3-hydroxy-4-((5-isopropylpyridin-3-yl)methylamino)-1-phenylbutan-2-yl)-N3-methyl-N3-((5-methyl-1,2,4-oxadiazol-3-yl)methyl)isophthalamide: ¹H NMR (300 MHz, CDCl₃+CD₃OD): 1.22 (d, 6H, J=6.9 Hz), 2.60 (s, 3H), 2.74-2.80 (m, 2H), 2.84-2.95 (m, 1H), 2.95-3.12 (m, 2H), 3.20-3.34 (m, 3H), 3.70-3.82 (m, 3H), 4.28-4.36 (m, 1H), 4.78-4.90 (m, 2H) 7.10-7.26 (m, 6H), 7.38-7.47 (m, 1H), 7.50-7.58 (m, 1H), 7.60-7.82 (m, 2H), 8.30 (m, 2H).

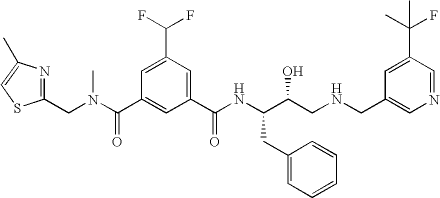

5-(difluoromethyl)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 8.454 (m, 2H), 7.724 (m, 4H), 7.201 (m, 5H), 6.902 (s, 1H), 6.610 (t, J=56.1 Hz, 1H), 4.943 (m, 0.7H), 4.605 (m, 0.3H), 4.364 (m, 1H), 3.811 (m, 3H), 2.976 (m, 5H), 2.786 (m, 2H), 2.437 (s, 3H), 1.666 (d, J=22.2, Hz, 6H).

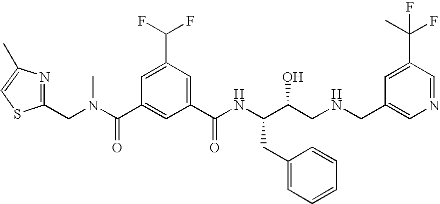

N1-((2S,3R)-4-((5-(1,1-difluoroethyl)pyridin-3-yl)methylamino)-3-hydroxy-1-phenylbutan-2-yl)-5-(difluoromethyl)-N3-methyl-N3-((4-methylthiazol-2-yl)methyl)isophthalamide: ¹H NMR (300 MHz, CDCl₃+CD₃OD), d: 8.602 (m, 2H), 7.808 (m, 3H), 7.631 (s, 1H), 7.161 (m, 5H), 6.911 (s, 1H), 6.620 (t, J=56.1, 111.9 Hz, 1H), 4.935 (m, 0.7H), 4.606 (m, 0.3H), 4.341 (m, 1H), 3.807 (m, 3H), 2.974 (m, 5H), 2.773 (m, 2H), 2.436 (s, 3H), 1.918 (t, J=36.5, 18.0 Hz, 3H).

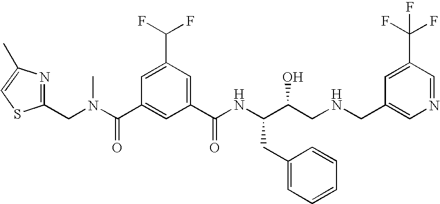

5-(difluoromethyl)-N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide: ¹H NMR (300 MHz, CDCl₃+CD₃OD), d: 8.602 (m, 2H), 7.829 (m 3H), 7.512 (m, 4H), 7.224 (m, 5H), 6.917 (s, 1H), 6.620 (t, J=56.1, 111.9 Hz, 1H), 4.973 (m, 0.7H), 4.621 (m, 0.3H), 4.370 (m, 1H), 3.847 (m, 3H), 3.006 (m, 5H), 2.799 (m, 2H), 2.458 (s, 3H).

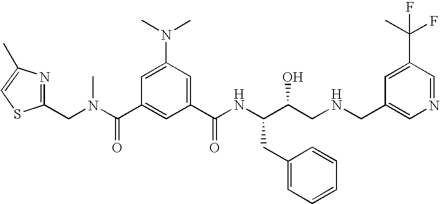

N1-((2S,3R)-4-((5-(1,1-difluoroethyl)pyridin-3-yl)methylamino)-3-hydroxy-1-phenylbutan-2-yl)-5-(dimethylamino)-N3-methyl-N3-((4-methylthiazol-2-yl)methyl)isophthalamide: ¹H NMR (300 MHz, CDCl₃+CD₃OD), d: 8.606 (d, J=9.6 Hz, 2 H), 7.846 (s, 1H), 7.176 (m, 5H), 6.894 (m, 4H), 4.922 (m, 1.2H), 4.660 (br, 0.8H), 4.314 (m, 1H), 3.843 (s, 3H), 3.739 (m, 1H), 3.045 (m, 2H), 2.939 (m, 9H), 2.753 (m, 2H), 2.437 (s, 3H), 1.929 (t, J=36.6, 18.3 Hz, 3H).

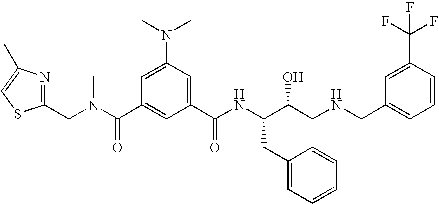

5-(dimethylamino)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide: ¹H NMR (300 MHz, CDCl₃+CD₃OD), d: 7.582-7.400 (m, 4H), 7.214 (m, 5H), 7.012-6.784 (m, 4H), 4.933 (m, 1.2H), 4.661 (br, 0.8H), 4.337 (m, 1H), 3.832 (m, 2H), 3.721 (m, 1H), 3.056 (m, 2H), 2.940 (m, 9H), 2.771 (m, 2H), 2.442 (s, 3 H).

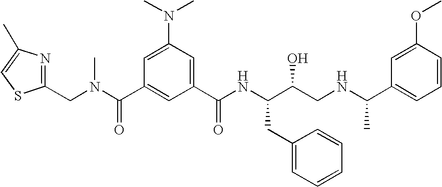

5-(dimethylamino)-N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)isophthalamide: ¹H NMR (300 MHz, CDCl₃+CD₃OD), d: 7.285 (m, 6H), 7.006-6.770 (m, 6H), 4.935 (m, 1.2H), 4.663 (br, 0.8H), 4.305 (m, 1H), 3.789 (s, 3H), 3.773 (m, 1H), 3.687 (br, 1H), 2.953 (m, 11H), 2.658 (m, 2H), 2.448 (s, 3H), 1.432 (d, J=6.9 Hz, 3 H).

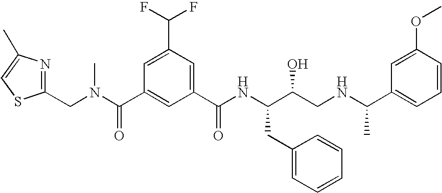

5-(difluoromethyl)-N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)isophthalamide: ¹H NMR (300 MHz, CDCl₃+CD₃OD), d: 7.836 (m, 3H), 7.207 (m, 6H), 6.921-6.459 (m, 5H), 4.955 (br, 1.4H), 4.619 (br, 0.6H), 4.344 (m, 1H), 3.782 (s, 3H), 3.763 (m, 2H), 2.989 (m, 5H), 2.674 (m, 2H), 2.451 (s, 3H), 1.431 (d, J=6.9 Hz, 3 H).

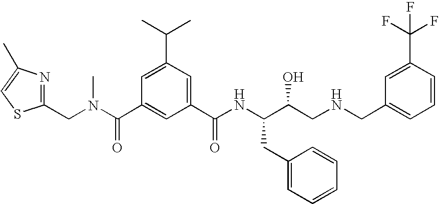

N1-((2S,3R)-3-hydroxy-1-phenyl-4-(3-(trifluoromethyl)benzylamino)butan-2-yl)-5-isopropyl-N3-methyl-N3-((4-methylthiazol-2-yl)methyl)isophthalamide: ¹H NMR (300 MHz, CDCl₃+CD₃OD), d: 7.507 (m 7H), 7.224 (m, 5H), 6.892 (s, 1H), 4.951 (m, 1.3H), 4.632 (br, 0.7H), 4.355 (m, 1H), 3.841 (m, 2H), 3.723 (m, 1H), 2.987 (m, 6H), 2.768 (m, 2H), 2.448 (s, 3H), 1.222 (d, J=5.7 Hz, 6H).

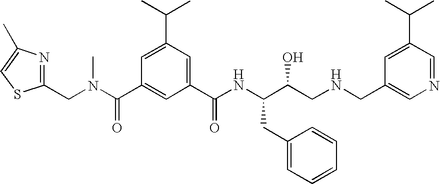

N1-((2S,3R)-3-hydroxy-4-((5-isopropylpyridin-3-yl)methylamino)-1-phenylbutan-2-yl)-5-isopropyl-N3-methyl-N3-((4-methylthiazol-2-yl)methyl)isophthalamide: ¹H NMR (300 MHz, CDCl₃+CD₃OD), d: 8.337 (m 2H), 7.496 (m, 4H), 7.225 (m, 5H), 6.896 (s, 1 H), 4.951 (m, 1.3H), 4.632 (br, 0.7H), 4.346 (m, 1H), 3.761 (m, 3H), 3.116-2.868 (m, 7H), 2.774 (m, 2H), 2.441 (s, 3H), 1.231 (m, 12H).

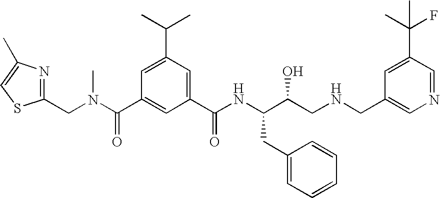

N1-((2S,3R)-4-((5-(2-fluoropropan-2-yl)pyridin-3-yl)methylamino)-3-hydroxy-1-phenylbutan-2-yl)-5-isopropyl-N3-methyl-N3-((4-methylthiazol-2-yl)methyl)isophthalamide: ¹H NMR (300 MHz, CDCl₃+CD₃OD), d: 8.437 (m 2H), 7.727-7.381 (m, 4H), 7.173 (m, 5 H), 6.887 (s, 1H), 4.932 (m, 1.3H), 4.621 (br, 0.7H), 4.325 (m, 1H), 3.813 (s, 2H), 3.765 (m, 1H), 3.096-2.879 (m, 6H), 2.768 (m, 2H), 2.424 (s, 3H), 1.663 (d, J=21.9 Hz, 6H), 1.195 (s, 6H).

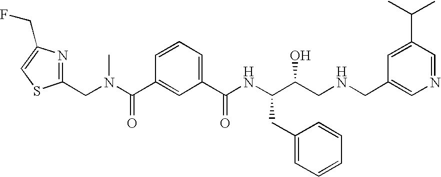

N1-((4-(fluoromethyl)thiazol-2-yl)methyl)-N3-((2S,3R)-3-hydroxy-4-((5-isopropylpyridin-3-yl)methylamino)-1-phenylbutan-2-yl)-N1-methylisophthalamide: ¹H NMR (300 MHz, CDCl₃+CD₃OD), d: 8.332 (m 2H), 7.755-7.371 (m, 4H), 7.202 (m, 5H), 5.446 (d, J=47.4 Hz, 2H), 4.973 (br, 1.3H), 4.690 (br, 0.7H), 4.366 (m, 1H), 3.777 (m, 3 H), 3.098-2.865 (m, 6H), 2.799 (m, 2H), 1.238 (m, 6H).

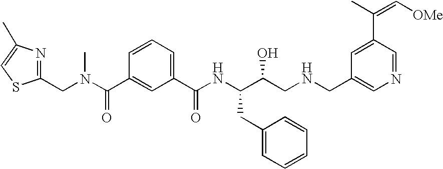

N1-((2S,3R)-3-hydroxy-4-((5-((Z)-1-methoxyprop-1-en-2-yl)pyridin-3-yl)methylamino)-1-phenylbutan-2-yl)-N3-methyl-N3-((4-methylthiazol-2-yl)methyl)isophthalamide: ¹H NMR (300 MHz, CDCl₃+CD₃OD), d: 8.686 (m, 0.6H), 8.377 (m, 0.4H), 8.266 (s, 1H), 7.902-7.307 (m, 5H), 7.151 (m, 5H), 6.865 (s, 1H), 6.461 (m, 0.4 H), 6.175 (m, 0.6H), 4.919 (br, 1.3H), 4.618 (br, 0.7H), 4.363 (m, 1H), 3.823-3.649 (m, 6 H), 2.956 (m, 5H), 2.789 (m, 2H), 2.416 (s, 3H), 1.896 (d, J=15.9 Hz, 3H).

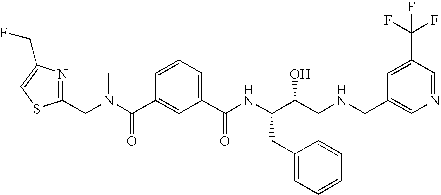

N1-((4-(fluoromethyl)thiazol-2-yl)methyl)-N3-((2S,3R)-3-hydroxy-1-phenyl-4-((5-(trifluoromethyl)pyridin-3-yl)methylamino)butan-2-yl)-N1-methylisophthalamide: ¹H NMR (300 MHz, CDCl₃+CD₃OD), d: 7.776 (s, 1H), 7.657-7.383 (m, 8H), 7.228 (m, 5H), 5.460 (d, J=47.7 Hz, 2H), 4.976 (br, 1.3H), 4.678 (br, 0.7H), 4.402 (m, 1H), 3.909-3.717 (m, 3 H), 3.013 (m, 5H), 2.798 (m, 2H).

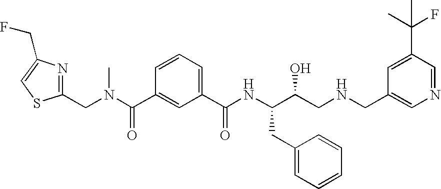

N1-((4-(fluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide: ¹H NMR (300 MHz, CDCl₃+CD₃OD), d: 8.459 (m 2H), 7.765-7.356 (m, 6H), 7.184 (m, 5H), 5.446 (d, J=47.4 Hz, 2H), 4.967 (br, 1.4H), 4.680 (br, 0.6H), 4.379 (m, 1H), 3.814 (m, 3 H), 3.105-2.903 (m, 5H), 2.795 (m, 2H), 1.6758 (d, J=22.2 Hz, 6H).

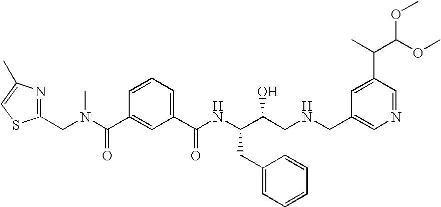

N1-((2S,3R)-4-((5-(1,1-dimethoxypropan-2-yl)pyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃+CD₃OD), d: 8.392 (m, 2H), 7.755-7.380 (m, 5H), 7.239 (m, 5 H), 6.893 (s, 1H), 4.966 (br, 1.3H), 4.650 (br, 0.7H), 4.402 (m, 1H), 4.336 (m, 1H), 3.808 (m, 3H), 3.378 (s, 3H), 3.261 (s, 3H), 3.019 (m, 6H), 2.812 (m, 2H), 2.457 (s, 3H), 1.272 (m, 3H).

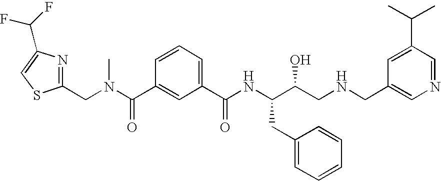

N1-((4-(difluoromethyl)thiazol-2-yl)methyl)-N3-((2S,3R)-3-hydroxy-4-((5-isopropylpyridin-3-yl)methylamino)-1-phenylbutan-2-yl)-N1-methylisophthalamide: ¹H NMR (300 MHz, CDCl₃+CD₃OD), d: 8.345 (s 2H), 7.771-7.378 (m, 6H), 7.227 (m, 5H), 6.753 (t, J=55.2 Hz, 1H), 4.978 (br, 1.4H), 4.705 (br, 0.6H), 4.390 (m, 1H), 3.786 (m, 3 H), 3.036-2.821 (m, 6H), 2.805 (m, 2H), 1.246 (d, J=6.9 Hz, 6H).

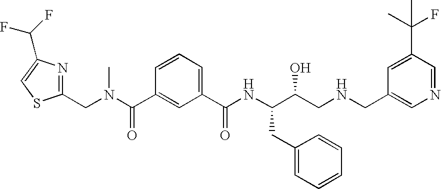

N1-((4-(difluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide: ¹H NMR (300 MHz, CDCl₃+CD₃OD), d: 8.464 (d, J=12.3 Hz, 2H), 7.751-7.364 (m, 6H), 7.214 (m, 5H), 6.741 (t, J=55.4 Hz, 1H), 4.960 (br, 1.5H), 4.693 (br, 0.5H), 4.375 (m, 1 H), 3.807 (m, 3H), 2.995 (m, 6H), 2.787 (m, 2H), 1.669 (d, J=22.2 Hz, 6H).

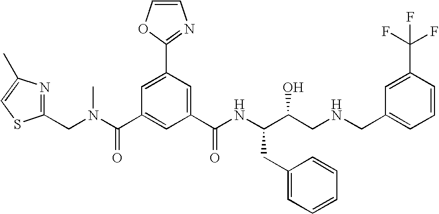

N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-2-yl)isophthalamide: ¹H NMR (300 MHz, CDCl₃+CD₃OD), d: 8.327-8.085 (m, 2H), 7.895-7.725 (m, 2H), 7.576-7.371 (m, 4H), 7.219 (m, 6H), 6.906 (s, 1H), 4.966 (br, 1.3H), 4.660 (br, 0.7H), 4.389 (m, 1H), 3.847 (m, 3H), 2.997 (m, 5H), 2.806 (m, 2H), 2.446 (s, 3H).

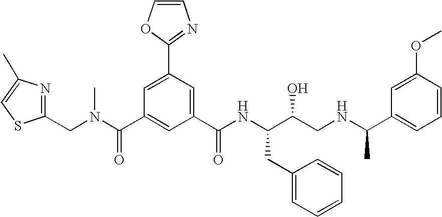

N1-((2S,3R)-3-hydroxy-4-((R)-1-(3-methoxyphenyl)ethylamino)-1-phenylbutan-2-yl)-N3-methyl-N3-((4-methylthiazol-2-yl)methyl)-5-(oxazol-2-yl)isophthalamide: ¹H NMR (300 MHz, CDCl₃+CD₃OD), d: 8.428 (s, 1.3H), 8.208 (s, 0.7H), 7.885 (s, 1H), 7.746 (s, 1 H), 7.260-7.106 (m, 7H), 6.933-6.748 (m, 4H), 4.972 (br, 1.3H), 4.684 (br, 0.7H), 4.391 (m, 1H), 3.737 (m, 5H), 3.041 (m, 4H), 2.866-2.637 (m, 3H), 2.452 (s, 3H), 1.438 (d, J=6.6 Hz, 3H).

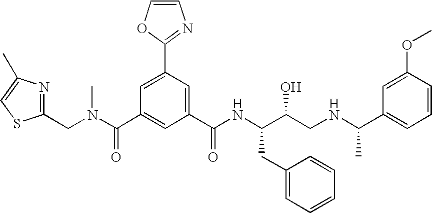

N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)-5-(oxazol-2-yl)isophthalamide: ¹H NMR (300 MHz, CDCl₃+CD₃OD), d: 8.342 (s, 1.3H), 8.181 (s, 0.7H), 7.801 (s, 1H), 7.747 (s, 1 H), 7.278-7.118 (m, 7H), 6.923-6.744 (m, 4H), 4.974 (br, 1.3H), 4.672 (br, 0.7H), 4.384 (m, 1H), 3.776 (m, 5H), 3.025 (m, 5H), 2.705 (m, 2H), 2.459 (s, 3H), 1.425 (d, J=6.3 Hz, 3H).

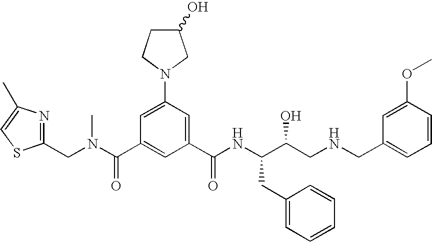

N1-((2S,3R)-3-hydroxy-4-(3-methoxybenzylamino)-1-phenylbutan-2-yl)-5-(3-hydroxypyrrolidin-1-yl)-N3-methyl-N3-((4-methylthiazol-2-yl)methyl)isophthalamide: ¹H NMR (300 MHz, CDCl₃) δ 7.33-7.16 (m, 9H), 7.00-6.83 (m, 6H), 6.74-6.60 (m, 1H), 4.97 (broad s, 1.1H), 4.67 (broad s, 0.8H), 4.55 (broad s, 1H), 4.36 (m, 1H), 3.97-3.79 (m, 6H), 3.38-3.27 (m, 4H), 3.12-2.81 (m, 8H), 2.48 (m, 3H), 2.07 (broad s, 3H).

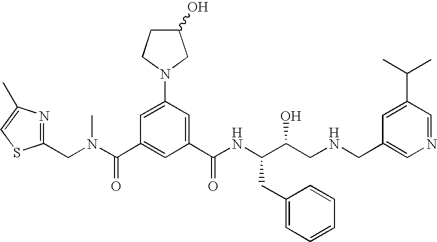

N1-((2S,3R)-3-hydroxy-4-((5-isopropylpyridin-3-yl)methylamino)-1-phenylbutan-2-yl)-5-(3-hydroxypyrrolidin-1-yl)-N3-methyl-N3-((4-methylthiazol-2-yl)methyl)isophthalamide: ¹H NMR (300 MHz, CDCl₃) δ 8.36 (s, 2H), 7.54 (s, 1H), 7.31-7.18 (m, 7H), 6.99-6.90 (m, 2H), 6.79-6.61 (m, 2H), 4.96 (broad s, 1.2H), 4.67 (broad s, 0.7H), 4.56 (broad s, 1H), 4.44-4.39 (m, 1H), 3.87-3.76 (m, 3H), 3.37-3.26 (m, 4H), 3.19-3.11 (m, 2H), 3.01-2.83 (m, 8H), 2.47 (m, 3H), 2.09 (broad s, 2H), 1.27-1.24 (m, 9H).

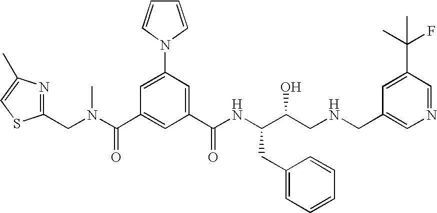

N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)-5-(1H-pyrrol-1-yl)isophthalamide: ¹H NMR (300 MHz, CDCl₃) δ 8.54-8.53 (m, 2H), 7.75-7.68 (m, 2H), 7.60-7.46 (m, 1H), 7.34-7.20 (m, 7H), 7.09 (s, 2H), 6.94 (m, 2H), 6.39 (s, 2H), 5.01 (broad s, 1.1H), 4.68 (broad s, 0.7H), 4.43-4.37 (m, 1H), 3.94-3.83 (m, 2H), 3.74 (m, 1H), 3.18-2.99 (m, 5H), 2.90-2.79 (m, 2H), 2.49 (s, 3H), 1.79-1.68 (m, 6H).

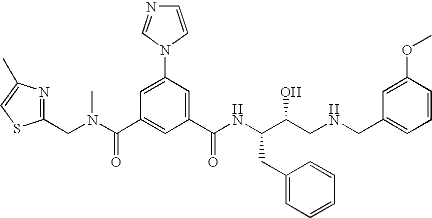

N1-((2S,3R)-3-hydroxy-4-(3-methoxybenzylamino)-1-phenylbutan-2-yl)-5-(1H-imidazol-1-yl)-N3-methyl-N3-((4-methylthiazol-2-yl)methyl)isophthalamide: ¹H NMR (300 MHz, CDCl₃) δ 8.20 (broad s, 1H), 7.96-7.84 (m, 2H), 7.71 (s, 1H), 7.57 (s, 1H), 7.32-7.15 (m, 8H), 6.95 (m, 2H), 6.84-6.81 (m, 1H), 4.99 (broad s, 1.0H), 4.65 (broad s, 0.8H), 4.44-4.39 (m, 1H), 4.05 (m, 1H), 3.86-3.77 (m, 5H), 3.17-3.04 (m, 4H), 2.99-2.88 (m, 2H), 2.48 (s, 3H).

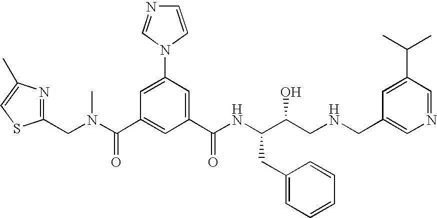

N1-((2S,3R)-3-hydroxy-4-((5-isopropylpyridin-3-yl)methylamino)-1-phenylbutan-2-yl)-5-(1H-imidazol-1-yl)-N3-methyl-N3-((4-methylthiazol-2-yl)methyl)isophthalamide: ¹H NMR (300 MHz, CDCl₃) δ 8.37 (d, 2H), 7.86-7.75 (m, 3H), 7.66 (m, 1H), 7.52 (m, 1H), 7.31-7.17 (m, 7H), 6.91 (d, 1H), 4.98 (broad s, 1.1H), 4.65 (broad s, 0.7H), 4.48-4.40 (m, 1H), 3.91-3.80 (m, 3H), 3.16-3.04 (m, 5H), 2.98-2.85 (m, 3H), 2.47 (s, 3H), 1.24 (d, 6H).

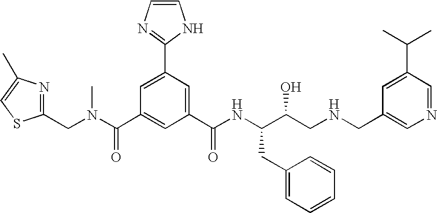

N1-((2S,3R)-3-hydroxy-4-((5-isopropylpyridin-3-yl)methylamino)-1-phenylbutan-2-yl)-5-(1H-imidazol-2-yl)-N3-methyl-N3-((4-methylthiazol-2-yl)methyl)isophthalamide: ¹H NMR (300 MHz, CDCl₃) δ 8.54 (s, 1H), 8.30 (s, 1H), 8.19-8.13 (m, 2H), 7.64-7.50 (m, 2H), 7.38-7.11 (m, 9H), 6.95-6.83 (m, 1H), 4.98 (ms, 1.4H), 4.65 (broad s, 0.4H), 4.41-4.35 (m, 1H), 4.14-4.07 (m, 1H), 3.76-3.71 (m, 2H), 3.13-2.85 (m, 6H), 2.75-2.71 (m, 2H), 2.48-2.42 (m, 3H), 1.23 (d, 6H).

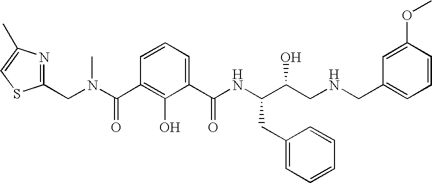

2-hydroxy-N1-((2S,3R)-3-hydroxy-4-(3-methoxybenzylamino)-1-phenylbutan-2-yl)-N3-methyl-N3-((4-methylthiazol-2-yl)methyl)isophthalamide: ¹H NMR (300 MHz, CDCl₃) δ 7.64-7.55 (m, 1H), 7.41-7.39 (m, 2H), 7.32-7.18 (m, 8H), 6.96-6.84 (m, 4H), 6.75 (m, 1H), 4.99-4.86 (broad m, 2H), 4.44-4.37 (m, 1H), 3.85-3.67 (m, 6H), 3.07-5.86 (m, 7H), 2.45 (s, 3H).

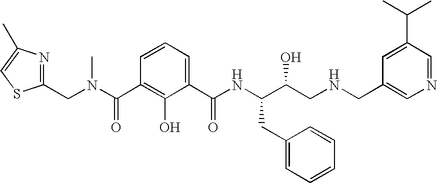

2-hydroxy-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.41 (m, 3H), 7.58-7.48 (m, 2H), 7.40-7.38 (m, 1H), 7.30-7.19 (m, 6H), 6.90 (s, 1H), 6.76 (m, 1H), 4.98 (broad s, 2H), 4.44-4.39 (m, 1H), 3.93-3.77 (m, 3H), 3.11-2.87 (m, 8H), 2.44 (s, 3H), 1.27 (d, 7H).

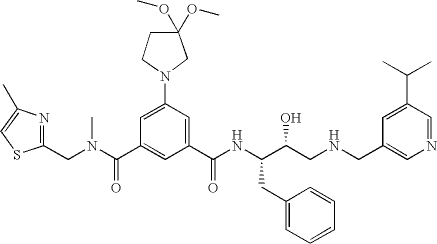

5-(3,3-dimethoxypyrrolidin-1-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.40 (s, 2H), 7.55 (m, 1H), 7.31-7.20 (m, 6H), 6.97-6.85 (m, 3H), 6.73-6.65 (m, 2H), 4.98 (broad s, 1.1H), 4.68 (broad s, 0.7H), 4.38-4.32 (m, 1H), 3.83 (m, 2H), 3.70 (broad s, 1H), 3.42-3.31 (m, 10H), 3.12-2.90 (m, 6H), 2.10 (s, 2H), 2.47 (s, 3H), 2.22 (m, 2H), 1.28 (d, 2H).

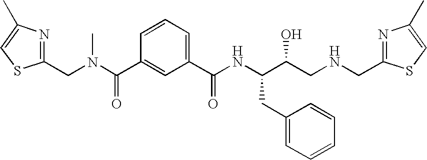

N1-((2S,3R)-3-hydroxy-4-((4-methylthiazol-2-yl)methylamino)-1-phenylbutan-2-yl)-N3-methyl-N3-((4-methylthiazol-2-yl)methyl)isophthalamide: ¹H NMR (300 MHz, CDCl₃) δ 7.83-7.64 (m, 2H), 7.57-7.55 (m, 1H), 7.45-7.40 (m, 1H), 7.35-7.19 (m, 5H), 7.06-7.04 (m, 1H), 6.91 (s, 1H), 6.84 (d, 1H), 4.99 (broad s, 1.2H), 4.66 (broad s, 0.6H), 4.50-4.41 (m, 1H), 4.13 (m, 2H), 3.75-3.73 (m, 1H), 3.14-3.01 (m, 5H), 2.48-2.43 (m, 6H).

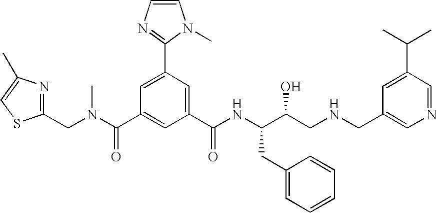

N1-((2S,3R)-3-hydroxy-4-((5-isopropylpyridin-3-yl)methylamino)-1-phenylbutan-2-yl)-N3-methyl-5-(1-methyl-1H-imidazol-2-yl)-N3-((4-methylthiazol-2-yl)methyl)isophthalamide: ¹H NMR (300 MHz, CDCl₃) δ 8.42-8.40 (m, 2H), 7.96-7.93 (m, 2H), 7.80-7.61 (m, 2H), 7.35-7.15 (m, 6H), 7.11 (m, 1H), 7.00 (m, 1H), 6.92-6.86 (m, 1H), 4.98 (broad s, 1.3H), 4.68 (broad s, 0.6H), 4.41-4.39 (m, 1H), 3.15-2.88 (m, 7H), 2.48-2.43 (m, 3H), 1.27 (d, 7H).

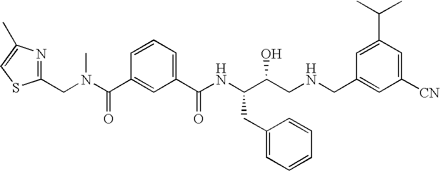

N1-((2S,3R)-4-(3-cyano-5-isopropylbenzylamino)-3-hydroxy-1-phenylbutan-2-yl)-N3-methyl-N3-((4-methylthiazol-2-yl)methyl)isophthalamide: ¹H NMR (300 MHz, CDCl₃): 1.25 (d, 6H, J=6.9 Hz), 2.48 (s, 3H), 2.89-3.13 (m, 8H), 3.85-4.02 (m, 3H), 4.23-4.4.38 (m, 1H), 4.62 (br s, 1H), 4.98 (br s, 1H), 6.91 (s, 1H), 7.17-7.39 (m, 8H), 7.64-7.67 (m, 2H), 7.75-7.77 (m, 2H).

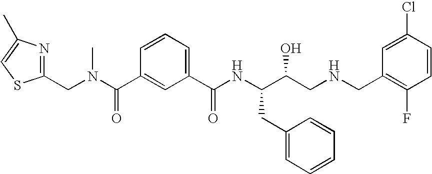

N1-((2S,3R)-4-(5-chloro-2-fluorobenzylamino)-3-hydroxy-1-phenylbutan-2-yl)-N3-methyl-N3-((4-methylthiazol-2-yl)methyl)isophthalamide: ¹H NMR (300 MHz, CDCl₃): 2.43 (s, 3H), 2.83 (br s, 3H), 2.98-3.22 (m, 5H), 3.62 (m, 1H), 3.80 (s, 2H), 4.36-4.42 (m, 1H), 4.64 (br s, 1H), 4.98 (br s, 1H), 6.98 (s, 1H), 7.12-7.98 (m, 10H).

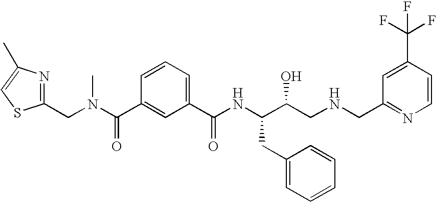

N1-((2S,3R)-3-hydroxy-1-phenyl-4-((4-(trifluoromethyl)pyridin-2-yl)methylamino)butan-2-yl)-N3-methyl-N3-((4-methylthiazol-2-yl)methyl)isophthalamide: ¹H NMR (300 MHz, CDCl₃): 2.42 (s, 3H), 2.84-3.18 (m, 7H), 3.68-3.81 (m, 1H), 4.02-4.18 (m, 2H), 4.42-4.55 (m, 1H), 4.65 (br s, 1H), 4.98 (br s, 1H), 6.98 (s, 1H), 7.21-7.96 (m, 11H), 8.78 (br s, 1H).

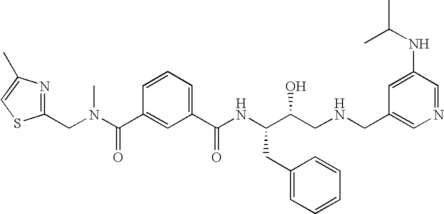

N1-((2S,3R)-3-hydroxy-4-((5-(isopropylamino)pyridin-3-yl)methylamino)-1-phenylbutan-2-yl)-N3-methyl-N3-((4-methylthiazol-2-yl)methyl)isophthalamide: ¹H NMR (300 MHz, CDCl₃): 1.22 (d, 6H, J=6.9 Hz), 2.42 (s, 3H0, 2.81-3.22 (m, 7H), 3.62-3.85 (m, 4H), 4.40-4.53 (m, 1H), 4.64 (br s, 1H), 4.98 (br s, 1H), 6.98 (s, 1H), 7.20-7.98 (m, 12H).

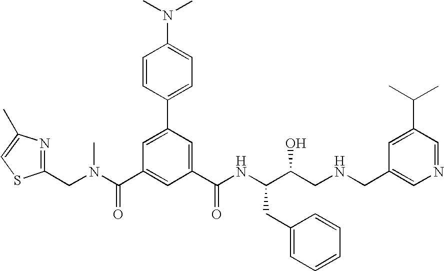

4′-(dimethylamino)-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide: ¹H NMR (300 MHz, CDCl₃): 1.25 (d, 6H, J=6.9 Hz), 2.47 (s, 3H), 2.82-3.14 (m, 14H), 3.78-3.89 (m, 3H), 4.37-4.42 (m, 1H), 4.69 (br s, 1H), 4.99 (br s, 1H), 6.77 (d, 1H, J=8.7 Hz), 6.91 (s, 1H), 7.20-7.30 (m, 6H), 7.43-7.85 (m, 6H), 8.37-8.39 (m, 2H).

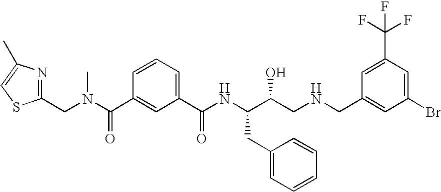

N1-((2S,3R)-4-(3-bromo-5-(trifluoromethyl)benzylamino)-3-hydroxy-1-phenylbutan-2-yl)-N3-methyl-N3-((4-methylthiazol-2-yl)methyl)isophthalamide: ¹H NMR (300 MHz, CDCl₃): 2.47 (s, 3H), 2.71-3.14 (m, 7H), 3.68-3.73 (m, 1H), 3.80-3.91 (m, 2H), 4.37-4.41 (m, 1H), 4.66 (br s, 1H), 4.98 (br s, 1H), 6.91 (br s, 2H), 7.21-7.73 (m, 11H).

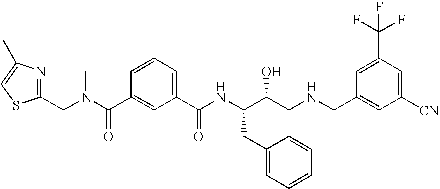

N1-((2S,3R)-4-(3-cyano-5-(trifluoromethyl)benzylamino)-3-hydroxy-1-phenylbutan-2-yl)-N3-methyl-N3-((4-methylthiazol-2-yl)methyl)isophthalamide: ¹H NMR (300 MHz, CDCl₃): 2.48 (s, 3H), 2.70-2.85 (m, 2H), 2.98-3.17 (m, 5H), 3.69-3.73 (m, 1H), 3.93 (s, 2H), 4.31-4.42 (m, 1H), 4.67 (br s, 1H), 4.99 (br s, 1H), 6.93 (s, 1H), 7.20-7.30 (m, 6H), 7.30-7.33 (m, 1H), 7.58-7.88 (m, 3H).

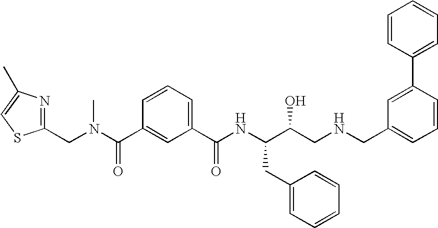

N1-((2S,3R)-4-(biphenyl-3-ylmethylamino)-3-hydroxy-1-phenylbutan-2-yl)-N3-methyl-N3-((4-methylthiazol-2-yl)methyl)isophthalamide: ¹H NMR (300 MHz, CDCl₃): 2.46 (s, 3H), 2.81-3.11 (m, 7H), 3.76-3.97 (m, 3H), 4.36-4.45 (m, 1H), 4.62 (br s, 1H), 4.96 (br s, 1H), 6.88 (s, 1H), 7.15-7.66 (m, 16 H0, 7.77 (s, 1H).

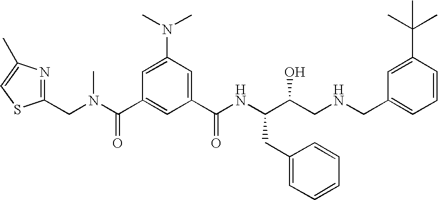

N1-((2S,3R)-4-(3-tert-butylbenzylamino)-3-hydroxy-1-phenylbutan-2-yl)-5-(dimethylamino)-N3-methyl-N3-((4-methylthiazol-2-yl)methyl)isophthalamide: ¹H NMR (300 MHz, CDCl₃): 2.47 (s, 3H), 2.82-3.12 (m, 13H), 3.77-3.95 (m, 3H), 4.31-4.39 (m, 1H), 4.67 (br s, 1H), 4.97 (br s, 1H), 6.78-7.07 (m, 3H), 7.18-7.41 (m, 10H).

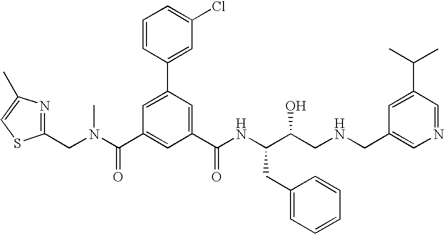

3′-chloro-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide: ¹H NMR (300 MHz, CDCl₃): 1.25 (d, 6H, J=6.9 Hz), 2.47 (s, 3H), 2.83-3.17 (m, 8H), 3.78-3.89 (m, 3H), 4.39-4.44 (m, 1H), 4.66 (br s, 1H), 4.99 (br s, 1H), 6.91 (s, 1H), 7.20-7.78 (m, 13H), 8.36-8.38 (m, 2H).

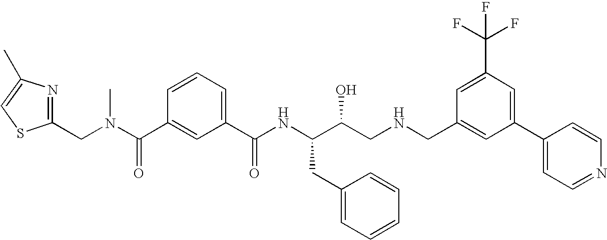

N1-((2S,3R)-3-hydroxy-1-phenyl-4-(3-(pyridin-4-yl)-5-(trifluoromethyl)benzylamino)butan-2-yl)-N3-methyl-N3-((4-methylthiazol-2-yl)methyl)isophthalamide: ¹H NMR (300 MHz, CDCl₃): 2.47 (s, 3H), 2.83-3.13 (m, 7H), 3.73 (m, 1H), 3.84-4.08 (m, 2H), 4.34-4.4.48 (m, 1H), 4.65 (br s, 1H), 4.97 (br s, 1H), 6.91 (s, 1H), 7.23-7.83 (m, 14H), 8.69-8.71 (m, 2H).

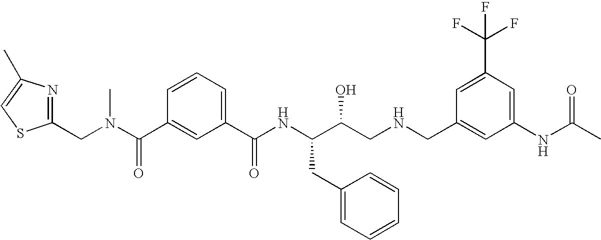

N1-((2S,3R)-4-(3-acetamido-5-(trifluoromethyl)benzylamino)-3-hydroxy-1-phenylbutan-2-yl)-N3-methyl-N3-((4-methylthiazol-2-yl)methyl)isophthalamide: ¹H NMR (300 MHz, CDCl₃): 2.10 (s, 3H), 2.47 (s, 3H), 2.81-3.12 (m, 7H), 3.71-3.86 (m, 3H), 4.38-4.48 (m, 1H), 4.62 (br s, 1H), 4.99 (br s, 1H), 6.91 (s, 1H), 7.19-7.38 (m, 7H), 7.49-7.69 (m, 3H), 7.87 (s, 1H), 8.99 (s, 1H).

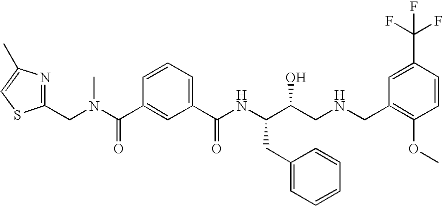

N1-((2S,3R)-3-hydroxy-4-(2-methoxy-5-(trifluoromethyl)benzylamino)-1-phenylbutan-2-yl)-N3-methyl-N3-((4-methylthiazol-2-yl)methyl)isophthalamide: ¹H NMR (300 MHz, CDCl₃): 2.47 (s, 3H), 2.79-3.14 (m, 7H), 3.73-3.92 (m, 6H), 4.38-4.48 (m, 1H), 4.66 (br s, 1H), 4.99 (br s, 1H), 6.91-6.95 (m, 2H)<7.20-7.39 (m, 7H), 7.52-7.64 (m, 3H), 7.75 (s, 1H).

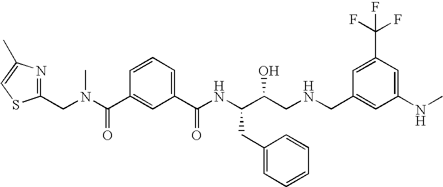

N1-((2S,3R)-3-hydroxy-4-(3-(methylamino)-5-(trifluoromethyl)benzylamino)-1-phenylbutan-2-yl)-N3-methyl-N3-((4-methylthiazol-2-yl)methyl)isophthalamide: ¹H NMR (300 MHz, CDCl₃): 2.47 (s, 3H), 2.82-3.14 (m, 10H), 3.71-3.85 (m, 3H), 4.36-4.44 (m, 1H), 4.64 (br s, 1H), 4.98 (br s, 1H), 6.68 (s, 1H), 6.75 (s, 1H), 6.87-6.91 (m, 2H), 7.17-7.39 (m, 7H), 7.52-7.76 (m, 2H).

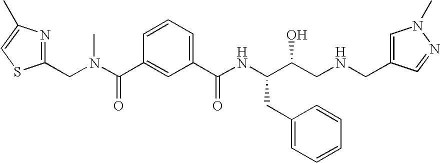

N1-((2S,3R)-3-hydroxy-4-((1-methyl-1H-pyrazol-4-yl)methylamino)-1-phenylbutan-2-yl)-N3-methyl-N3-((4-methylthiazol-2-yl)methyl)isophthalamide: ¹H NMR (300 MHz, CDCl₃): 2.47 (s, 3H), 2.82-3.18 (m, 7H), 3.82-4.00 (m, 6H), 4.36-4.39 (m, 1H), 4.62 (br s, 1H), 4.98 (br s, 1H), 6.92 (s, 1H), 7.15-7.30 (m, 6H), 7.38-7.57 (m, 3H), 7.75-7.81 (m, 2H).

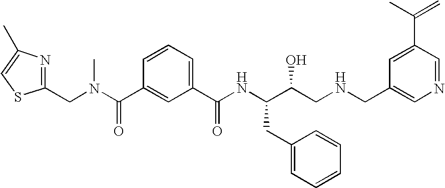

N1-((2S,3R)-3-hydroxy-1-phenyl-4-(5-(prop-1-en-2-yl)nicotinamido)butan-2-yl)-N3-methyl-N3-((4-methylthiazol-2-yl)methyl)isophthalamide: ¹H NMR (300 MHz, CDCl₃): 2.19 (s, 3H), 2.48 (s, 3H), 3.00-3.18 (m, 5H), 3.33-3.41 (m, 2H), 3.82-3.98 (m, 1H), 4.36-4.44 (m, 1H), 4.62 (br s, 1H), 4.98 (br s, 1H), 5.25 (s, 1H), 5.52 (s, 1H), 6.95 (s, 1H), 7.21-7.64 (m, 9H), 8.36 (m, 1H), 8.85 (s, 1H), 9.07 (s, 1H).

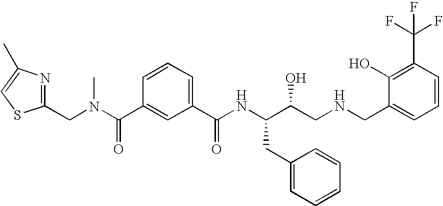

N1-((2S,3R)-3-hydroxy-4-(2-hydroxy-3-(trifluoromethyl)benzylamino)-1-phenylbutan-2-yl)-N3-methyl-N3-((4-methylthiazol-2-yl)methyl)isophthalamide: ¹H NMR (300 MHz, CDCl₃): 2.47 (s, 3H), 2.78-3.12 (m, 7H), 3.95-4.14 (m, 3H), 4.26-4.38 (m, 1H), 4.64 (br s, 1H), 4.96 (br s, 1H), 6.80-6.90 (m, 3H), 7.14-7.47 (m, 8H), 7.61-7.69 (m, 2H).

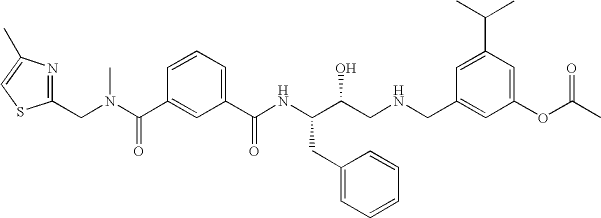

3-(((2R,3S)-2-hydroxy-3-(3-(methyl((4-methylthiazol-2-yl)methyl)carbamoyl)benzamido)-4-phenylbutylamino)methyl)-5-isopropylphenyl acetate: ¹H NMR (300 MHz, CDCl₃) δ 7.72-7.86 (m, 1H), 7.67-7.70 (m, 1H), 7.56-7.65 (m, 1H), 7.42-7.47 (m, 1H), 7.19-7.35 (m, 6H), 7.08 (s, 1H), 6.86-6.98 (m, 4H), 5.00 (s, 2H), 4.37-4.47 (m, 1H), 3.79-3.90 (m, 2H), 3.66-3.75 (m, 1H), 2.97-3.21 (m, 5H), 2.79-2.96 (m, 3H), 2.48 (s, 3H), 2.31 (s, 3H), 1.24-1.26 (m, 6H).

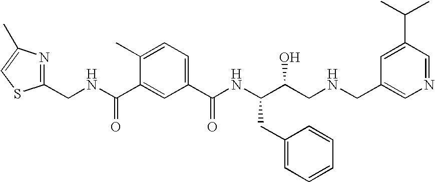

N1-((2S,3R)-3-hydroxy-4-((5-isopropylpyridin-3-yl)methylamino)-1-phenylbutan-2-yl)-4-methyl-N3-((4-methylthiazol-2-yl)methyl)isophthalamide: ¹H NMR (300 MHz, CDCl₃) δ 8.63-8.68 (m, 1H), 8.43 (s, 1H), 8.18-8.21 (m, 1H), 7.97 (s, 1H), 7.44-7.45 (m, 1H), 7.38-7.44 (m, 1H), 7.20-7.38 (m, 7H), 6.857-6.861 (m, 1H), 6.57-6.72 (m, 1H), 4.86-5.00 (m, 2H), 4.39-4.48 (m, 1H), 3.97-4.02 (m, 1H), 3.61-3.72 (m, 2H), 3.07-3.09 (m, 2H), 2.80-2.94 (m, 1H), 2.61-2.74 (m, 2H), 2.56 (s, 3H), 2.440-2.442 (m, 3H), 1.21-1.24 (m, 6H).

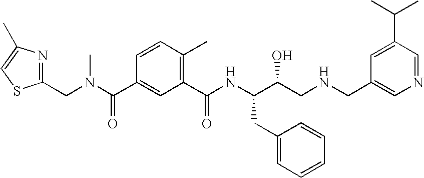

N3-((2S,3R)-3-hydroxy-4-((5-isopropylpyridin-3-yl)methylamino)-1-phenylbutan-2-yl)-N1,4-dimethyl-N1-((4-methylthiazol-2-yl)methyl)isophthalamide: ¹H NMR (300 MHz, CDCl₃) δ 8.41 (s, 2H), 7.56 (s, 1H), 7.33-7.46 (m, 1H), 7.10-7.33 (m, 8H), 6.86-6.97 (m, 1H), 6.06-6.26 (m, 1H), 4.87-5.06 (m, 2H), 4.34-4.47 (m, 1H), 3.85 (s, 2H), 3.64-3.76 (m, 1H), 3.19-3.26 (m, 1H), 2.90-3.17 (m, 4H), 2.78-2.91 (m, 4H), 2.48 (s, 3H), 2.20 (s, 3H), 1.28-1.31 (m, 6H).

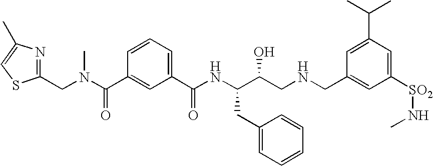

N1-((2S,3R)-3-hydroxy-4-(3-isopropyl-5-(N-methylsulfamoyl)benzylamino)-1-phenylbutan-2-yl)-N3-methyl-N3-((4-methylthiazol-2-yl)methyl)isophthalamide: ¹H NMR (300 MHz, CDCl₃) δ 7.74-7.86 (m, 1H), 7.69-7.74 (m, 2H), 7.66-7.69 (m, 1H), 7.64 (s, 1H), 7.51-7.60 (m, 1H), 7.37-7.46 (m, 2H), 7.16-7.33 (m, 5H), 6.97-7.16 (m, 1H), 6.92 (s, 1H), 5.30-5.60 (s, 1H), 4.99 (s, 2H), 4.34-4.43 (m, 1H), 3.80-3.94 (m, 2H), 3.71-3.81 (m, 1H), 3.10-3.19 (m, 1H), 3.02 (s, 3H), 2.88-3.10 (m, 3H), 2.81-2.82 (m, 2H), 2.60 (s, 3H), 2.48 (s, 3H), 1.26-1.28 (m, 6H).

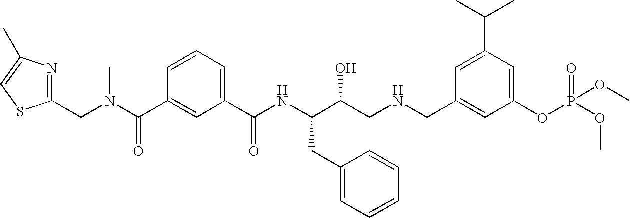

3-(((2R,3S)-2-hydroxy-3-(3-(methyl((4-methylthiazol-2-yl)methyl)carbamoyl)benzamido)-4-phenylbutylamino)methyl)-5-isopropylphenyl dimethyl phosphate: ¹H NMR (300 MHz, CDCl₃) δ 7.75-7.89 (m, 1H), 7.70-7.72 (m, 1H), 7.56-7.68 (m, 1H), 7.42-7.47 (m, 1H), 7.18-7.40 (m, 6H), 7.11 (s, 1H), 7.06 (s, 1H), 7.00 (s, 1H), 6.85-6.97 (m, 1H), 5.00 (s, 2H), 4.37-4.46 (m, 1H), 3.89-3.91 (m, 3H), 3.86-3.87 (m, 3H), 3.69-3.79 (m, 1H), 2.97-3.20 (m, 7H), 2.81-2.96 (m, 4H), 2.49 (s, 3H), 1.23-1.26 (m, 6H).

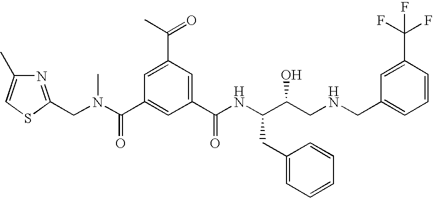

5-acetyl-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide: ¹H NMR (300 MHz, CDCl₃) δ 8.22 (s, 1H), 8.09 (s, 1H), 7.94 (s, 1H), 7.42-7.60 (m, 4H), 7.19-7.29 (m, 5H), 6.91 (s, 1H), 4.90 (m, 2H), 4.43 (m, 1H), 3.85-3.99 (m, 2H), 3.75 (m, 1H), 2.99-3.16 (m, 5H), 2.83 (m, 2H), 2.60 (s, 3H), 2.47 (s, 3H).

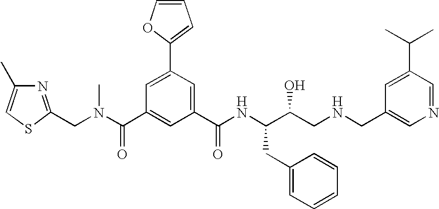

5-(furan-2-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.35-8.37 (m, 2H), 7.90 (s, 1H), 7.74 (s, 1H), 7.54 (m, 2H), 7.46 (m, 1H), 7.18-7.29 (m, 5H), 6.91 (s, 1H), 6.66 (s, 1H), 6.49 (m, 1H), 4.98 (m, 2H), 4.41 (m, 1H), 3.77-3.90 (m, 3H), 3.01-3.14 (m, 5H), 2.89 (m, 1H), 2.82 (m, 2H), 2.47 (s, 3H), 1.21 (m, 6H).

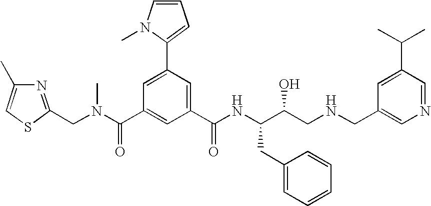

N1-((2S,3R)-3-hydroxy-4-((5-isopropylpyridin-3-yl)methylamino)-1-phenylbutan-2-yl)-N3-methyl-5-(1-methyl-1H-pyrrol-2-yl)-N3-((4-methylthiazol-2-yl)methyl)isophthalamide: ¹H NMR (300 MHz, CDCl₃) δ 8.35 (m, 2H), 7.73 (s, 1H), 7.53-7.62 (m, 2H), 7.15-7.29 (m, 6H), 6.90 (s, 1H), 6.74 (s, 1H), 6.21-6.24 (m, 2H), 4.98 (m, 2H), 4.39 (m, 1H), 3.71-3.87 (m, 3H), 3.64 (s, 3H), 3.00-3.18 (m, 5H), 2.89 (m, 1H), 2.81 (m, 2H), 2.46 (s, 3H), 1.24 (m, 6H).

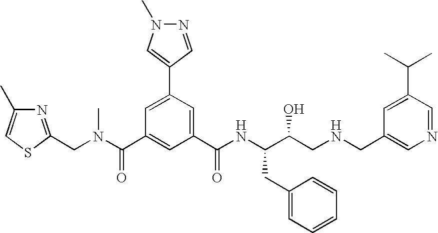

N1-((2S,3R)-3-hydroxy-4-((5-isopropylpyridin-3-yl)methylamino)-1-phenylbutan-2-yl)-N3-methyl-5-(1-methyl-1H-pyrazol-4-yl)-N3-((4-methylthiazol-2-yl)methyl)isophthalamide: ¹H NMR (300 MHz, CDCl₃) δ 8.41 (m, 1H), 8.35 (m, 1H), 7.57-7.79 (m, 6H), 7.19-7.26 (m, 5H), 6.91 (s, 1H), 4.98 (m, 2H), 4.41 (M, 1H), 3.82-3.93 (m, 6H), 2.85-3.12 (m, 8H), 2.46 (s, 3H), 1.21 (m, 6H).

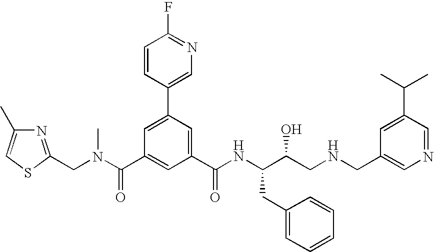

5-(6-fluoropyridin-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.38 (m, 3H), 7.95-7.98 (m, 1H), 7.84 (s, 1H), 7.70 (s, 1H), 7.52 (s, 1H), 7.21-7.28 (m, 6H), 7.05 (m, 1H), 6.92 (s, 1H), 5.00 (m, 2H), 4.43 (m, 1H), 3.76-3.90 (m, 3H), 3.05-3.18 (m, 5H), 2.92 (m, 1H), 2.82 (m, 2H), 2.48 (s, 3H), 1.24 (m, 6H).

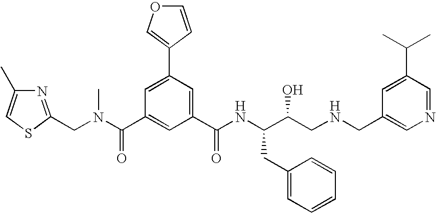

5-(furan-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide: ¹H NMR (300 MHz, CDCl₃) δ 8.35-8.38 (m, 2H), 7.71 (m, 2H), 7.55 (s, 2H), 7.48 (s, 1H), 7.20-7.26 (m, 6H), 6.91 (s, 1H), 6.64 (s, 1H), 4.98 (m, 2H), 4.40 (m, 1H), 3.79-3.89 (m, 3H), 3.00-3.14 (m, 5H), 2.90 (m, 1H), 2.83 (m, 2H), 2.47 (s, 3H), 1.22 (m, 6H).

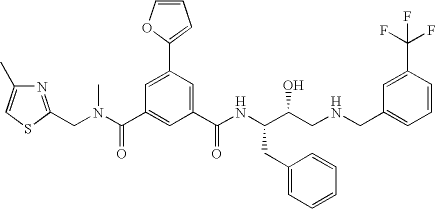

5-(furan-2-yl)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide: ¹H NMR (300 MHz, CDCl₃) δ 7.90 (s, 1H), 7.77 (s, 1H), 7.41-7.61 (m, 6H), 7.19-7.29 (m, 5H), 6.92 (s, 1H), 6.69 (s, 1H), 6.50 (m, 1H), 5.00 (m, 2H), 4.41 (m, 1H), 3.89 (m, 2H), 3.74 (m, 1H), 3.03-3.16 (m, 5H), 2.82 (s, 2H), 2.48 (s, 3H).

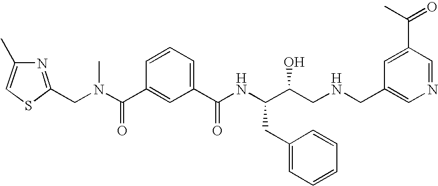

N1-((2S,3R)-4-((5-acetylpyridin-3-yl)methylamino)-3-hydroxy-1-phenylbutan-2-yl)-N3-methyl-N3-((4-methylthiazol-2-yl)methyl)isophthalamide: ¹H NMR (300 MHz, CDCl₃) δ 9.02 (m, 1H), 8.72 (m, 1H), 8.20 (m, 1H), 7.71 (m, 1H), 7.63 (m, 1H), 7.33 (m, 1H), 7.17-7.29 (m, 6H), 6.88 (s, 1H), 4.94 (m, 2H), 4.37 (m, 1H), 3.87 (m, 2H), 3.72 (m, 1H), 2.98-3.12 (m, 5H), 2.79 (m, 2H), 2.62 (s, 3H), 2.44 (s, 3H).

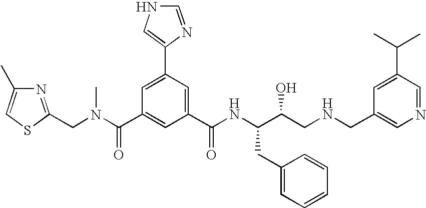

N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrazol-4-yl)isophthalamide: ¹H NMR (300 MHz, CDCl₃) δ 8.41 (s, 2H), 7.80 (s, 1H), 7.62 (m, 1H), 7.56 (s, 1H), 7.21-7.29 (m, 8H), 6.93 (s, 1H), 4.98 (m, 2H), 4.39 (m, 1H), 3.82-3.93 (m, 2H), 3.76 (m, 1H), 3.02-3.18 (m, 5H), 2.95 (m, 1H), 2.84 (s, 2H), 2.48 (s, 3H), 1.27 (m, 6H).

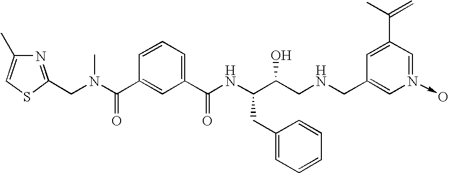

3-(((2R,3S)-2-hydroxy-3-(3-(methyl((4-methylthiazol-2-yl)methyl)carbamoyl)benzamido)-4-phenylbutylamino)methyl)-5-(prop-1-en-2-yl)pyridine 1-oxide: ¹H NMR (300 MHz, CDCl₃) δ 8.17 (m, 2H), 7.77 (m, 1H), 7.67 (m, 1H), 7.49 (m, 1H), 7.34 (m, 2H), 7.15-7.24 (m, 5H), 6.88 (s, 1H), 5.45 (s, 1H), 5.24 (s, 1H), 4.94 (m, 2H), 4.39 (m, 1H), 3.76-3.80 (m, 3H), 2.97-3.11 (m, 5H), 2.75 (m, 2H), 2.44 (s, 3H), 2.07 (s, 3H).

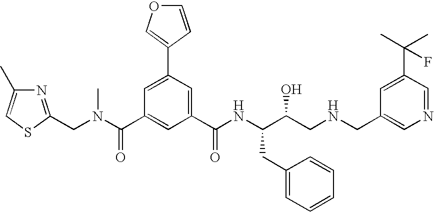

N1-((2S,3R)-4-((5-(2-fluoropropan-2-yl)pyridin-3-yl)methylamino)-3-hydroxy-1-phenylbutan-2-yl)-5-(furan-3-yl)-N3-methyl-N3-((4-methylthiazol-2-yl)methyl)isophthalamide: ¹H NMR (300 MHz, CDCl₃) δ 8.49-8.51 (m, 2H), 7.69-7.73 (m, 3H), 7.47-7.52 (m, 3H), 7.18-7.29 (m, 5H), 6.91 (s, 1H), 6.61 (s, 1H), 4.98 (m, 2H), 4.40 (m, 1H), 3.79-3.90 (m, 3H), 2.99-3.14 (m, 5H), 2.82 (s, 2H), 2.46 (s, 3H), 1.71 (m, 3H), 1.64 (m, 3H).

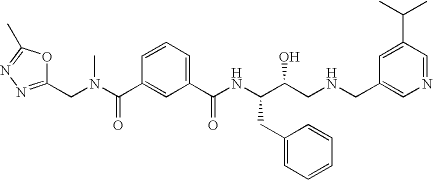

N1-((2S,3R)-3-hydroxy-4-((5-isopropylpyridin-3-yl)methylamino)-1-phenylbutan-2-yl)-N3-methyl-N3-((5-methyl-1,3,4-oxadiazol-2-yl)methyl)isophthalamide: ¹H NMR (300 MHz, CDCl₃+CD₃OD): 1.25 (d, 6H, J=6.6 Hz), 2.18-2.60 (m, 3H), 2.58 (s, 3H), 2.82 (d, 2H, J=4.5 Hz), 2.84-3.06 (m, 3H), 3.74-3.86 (m, 3H), 4.36-4.50 (m, 1H), 4.88-5.00 (m, 2H) 7.16-7.30 (m, 6H), 7.40-7.88 (m, 4H), 8.38 (br s, 2H).

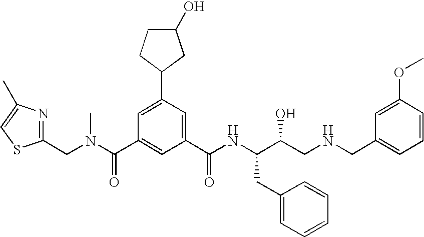

N1-((2S,3R)-3-hydroxy-4-(3-methoxybenzylamino)-1-phenylbutan-2-yl)-5-(3-hydroxycyclopentyl)-N3-methyl-N3-((4-methylthiazol-2-yl)methyl)isophthalamide.

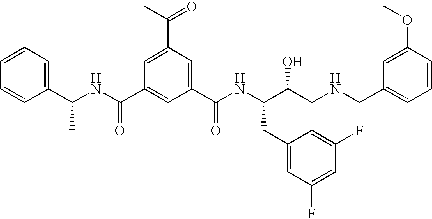

5-acetyl-N1-((2S,3R)-1-(3,5-difluorophenyl)-3-hydroxy-4-(3-methoxybenzylamino)butan-2-yl)-N3-((R)-1-phenylethyl)isophthalamide.

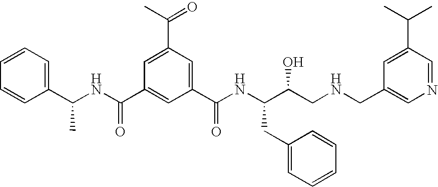

5-acetyl-N1-((2S,3R)-3-hydroxy-4-((5-isopropylpyridin-3-yl)methylamino)-1-phenylbutan-2-yl)-N3-((R)-1-phenylethyl)isophthalamide.

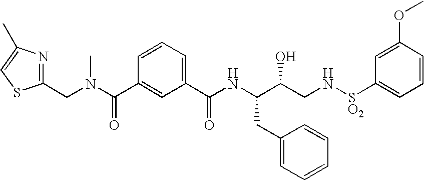

N1-((2S,3R)-3-hydroxy-4-(3-methoxyphenylsulfonamido)-1-phenylbutan-2-yl)-N3-methyl-N3-((4-methylthiazol-2-yl)methyl)isophthalamide.

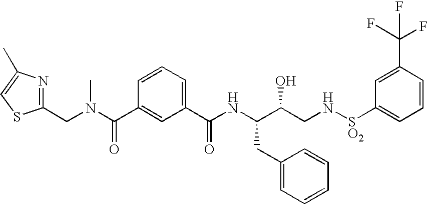

N1-((2S,3R)-3-hydroxy-1-phenyl-4-(3-(trifluoromethyl)phenylsulfonamido)butan-2-yl)-N3-methyl-N3-((4-methylthiazol-2-yl)methyl)isophthalamide.

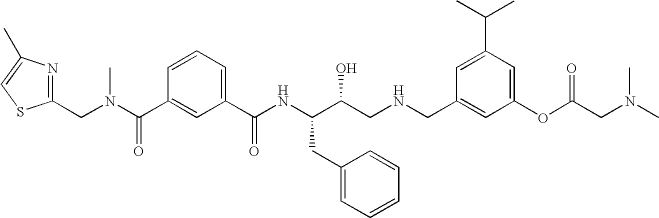

3-(((2R,3S)-2-hydroxy-3-(3-(methyl((4-methylthiazol-2-yl)methyl)carbamoyl)benzamido)-4-phenylbutylamino)methyl)-5-isopropylphenyl 2-(dimethylamino)acetate.

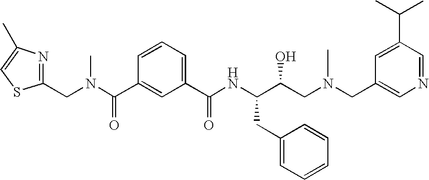

N1-((2S,3R)-3-hydroxy-4-(((5-isopropylpyridin-3-yl)methyl)(methyl)amino)-1-phenylbutan-2-yl)-N3-methyl-N3-((4-methylthiazol-2-yl)methyl)isophthalamide.

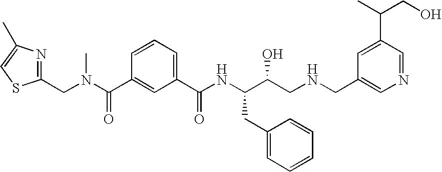

N1-((2S,3R)-3-hydroxy-4-((5-(1-hydroxypropan-2-yl)pyridin-3-yl)methylamino)-1-phenylbutan-2-yl)-N3-methyl-N3-((4-methylthiazol-2-yl)methyl)isophthalamide.

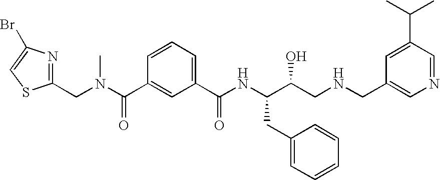

N1-((4-bromothiazol-2-yl)methyl)-N3-((2S,3R)-3-hydroxy-4-((5-isopropylpyridin-3-yl)methylamino)-1-phenylbutan-2-yl)-N1-methylisophthalamide.

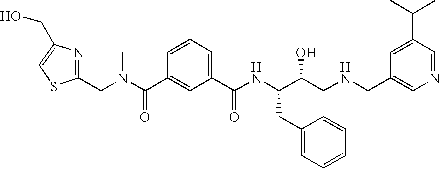

N1-((2S,3R)-3-hydroxy-4-((5-isopropylpyridin-3-yl)methylamino)-1-phenylbutan-2-yl)-N3-((4-(hydroxymethyl)thiazol-2-yl)methyl)-N3-methylisophthalamide.

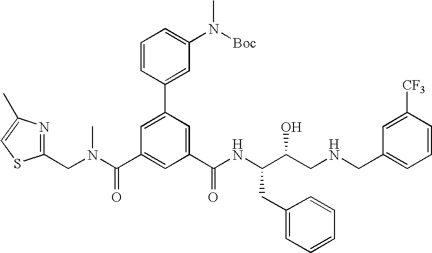

tert-butyl 3′-((2S,3R)-3-hydroxy-1-phenyl-4-(3-(trifluoromethyl)benzylamino)butan-2-ylcarbamoyl)-5′-(methyl((4-methylthiazol-2-yl)methyl)carbamoyl)biphenyl-3-yl(methyl)carbamate.

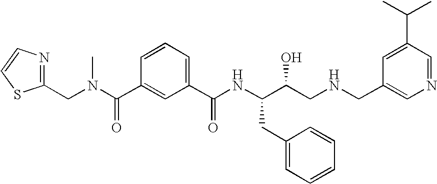

N1-((2S,3R)-3-hydroxy-4-((5-isopropylpyridin-3-yl)methylamino)-1-phenylbutan-2-yl)-N3-methyl-N3-(thiazol-2-ylmethyl)isophthalamide.

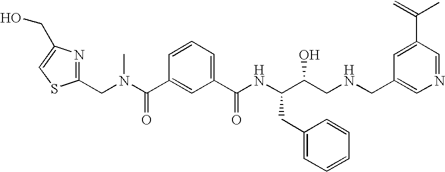

N1-((2S,3R)-3-hydroxy-1-phenyl-4-((5-(prop-1-en-2-yl)pyridin-3-yl)methylamino)butan-2-yl)-N3-((4-(hydroxymethyl)thiazol-2-yl)methyl)-N3-methylisophthalamide.

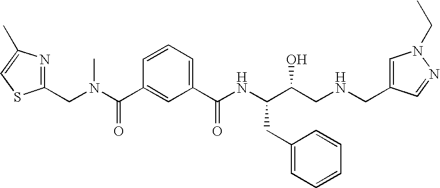

N1-((2S,3R)-4-((1-ethyl-1H-pyrazol-4-yl)methylamino)-3-hydroxy-1-phenylbutan-2-yl)-N3-methyl-N3-((4-methylthiazol-2-yl)methyl)isophthalamide.

### Example 4

### Inhibition of Memapsin 1 Beta-Secretase Activity and Cathepsin D Activity

A substrate peptide NH₃-ELDLAVEFWHDR-CO₂ (SEQ ID NO.: 1) was dissolved at 2 mg/mL in 10% glacial acetic acid and diluted into 0.009M NaOH to obtain μM concentration at pH 4.1. After equilibration at 37 degrees C., the reactions were initiated by the addition of an aliquot of memapsin 2. Aliquots were removed at time intervals, and combined with an equal volume of MALDI-TOF matrix (α-hydroxycinnamic acid in acetone, 20 mg/mL) and immediately spotted in duplicate onto a stainless-steel MALDI sample plate. MALDI-TOF mass spectrometry was performed on a PE Biosystems Voyager DE. The instrument was operated at 25,000 accelerating volts in positive mode with a 150 ns delay. Ions with a mass-to-charge ratio (m/z) were detected in the range of 650-2000 atomic mass units. Data were analyzed by the Voyager Data Explorer module to obtain ion intensity data for mass species of substrates and corresponding products in a given mixture. Relative product formation was calculated as the ratio of signal intensity of the product to the sum of signal intensities of both product and the corresponding substrate. Relative product formed per unit time was obtained from non-linear regression analysis of the data representing the initial 15% formation of product using the model:

1−e^{-kT},

where k was the relative hydrolytic rate constant and T was time in seconds. Initial rates were expressed relative to uninhibited controls and fit to a tight-binding model of competitive inhibition as above. Results are shown in Table 1 above.

### Example 5

### Cellular AβIC50 Determinations

The potency of compounds against memapsin 2 activity was determined in a cellular assay of Aβ production. Compounds that successfully penetrate the cell membrane demonstrated their ability to inhibit memapsin 2 activity in endosomal compartments, thus blocking the production of Aβ. Chinese hamster ovary cells that over-express human APP695 with the London and Swedish mutations were seeded in multi-well plates at 10% confluency. Compounds are dissolved in DMSO to concentrations near 1 mM, and diluted into culture media to a final concentration near 4 μM (final 0.4% DMSO). Compounds were diluted serially and applied to cells in multi-well plates 48 h after seeding. Incubation was continued in 5% CO₂ at 37° C. for 24 h. Aliquots were removed and assayed for Aβ₄₀ content using a sandwich ELISA (BioSource International). Amount of Aβ₄₀ over the range of concentration of compounds, relative to control incubations, were fit to a 4-parameter IC₅₀ model. Results are provided in Table 1 above.

### Example 6

### Quantitative Analysis of Inhibitors in Biological Samples by Triple Quadrupole Mass Spectrometry

Quantitative analysis was used to determine the concentration of a test inhibitor, N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide, in biological tissues. The test inhibitor was extracted from a variety of biological samples followed by its selective identification and quantification from a standard curve prepared in the same extracted biological matrix. Briefly, this extraction is accomplished through a simple protein precipitation technique performed on collected plasma. The resultant extractions are then quantified using the LC/MS protocol as detailed herein.

Whole blood from animals dosed with the test inhibitor was collected in lithium heparin anti-coagulant tubes. These samples were centrifuged to allow the subsequent collection of plasma, with plasma aliquots immediately stored at −70° C. For quantitative analysis, the samples were then thawed on ice and a 20 μl aliquot was added to a well of a multi-screen Solvinert filter plate (Millipore). A five-fold volume of acetonitrile was added to each sample to facilitate protein precipitation, the samples were multiply aspirated or shaken to mix and then centrifuged at 4500 rpm for 15 minutes to filter the samples, with the supernatant being collected in an underlying 96 well receiving plate. The receptor plate was then dried via speed vacuum at 40° C. The sample was reconstituted by multiple aspirations with a reconstitution buffer composed of 10% acetonitrile, 10% DMSO, 80% H₂O with an internal standard added at a concentration of 100 ng/ml. Standards of the test inhibitor were prepared in 100% DMSO for the generation of a standard curve for sample quantification. The samples range from 1 to 100000 ng/ml, as required for the analysis. A 1 μl aliquot of standard was added per 10 μl of blank plasma to construct a calibration curve, 0.1 ng/ml to 10000 ng/ml, prior to the protein precipitation step again as required for analysis. Quality control samples were prepared identically, from an alternate standard source and a single sample was prepared with an identical volume spike added post precipitation and collection to monitor the extraction efficiency. This method was applicable to plasma from all species currently being tested. Solvinert filter wells were pre-washed with 100 μl of 10% MeOH/90% ACN followed by centrifugation at 4500 RPM for 10 min prior to use.

Quantitative analysis was performed through LC/MS analysis. The system was composed of an Eksigent Express LC 100 microflow HPLC system, coupled with an Eksigent CXP C-8AR 0.3*50 mm, or other suitable column, and a Thermo TSQuantum Classic triple quadrupole mass spectrometer running in positive ion, SRM mode. The HPLC buffers were 99.5% H₂O, 0.5% formic acid (buffer A) and 99.5% acetonitrile, 0.5% formic acid (buffer B). Typically, a 10 μl aliquot was injected onto the column followed by a 1.5 minute chromatographic analysis. This analysis was conducted at a 20 μl/min flow rate, and composed of a gradient from 15% B to 90% B followed by a 98% B flush to facilitate compound separation and quantitation. Each sample was analyzed in duplicate, with QC, and system blank samples run after every sample set. The column effluent was analyzed in real time by MS/MS to allow the positive identification and quantification of the test inhibitor by compound fingerprint analysis. This was accomplished by allowing only species of 586.3 Da to pass through the first quadrupole, followed by collision induced decay in quadrupole two and scanning analysis of selected daughter ions specific to the test compound in quadrupole three. The collected data was analyzed using the LCQuan software package from Thermo Electron, to determine the levels of the test compound in the samples.

### Example 7

### Plasma and Brain Pharmacokinetic Analysis

Male Sprague-Dawley Rats (200-400 g) were obtained from Taconic Farms (Hudson, N.Y.) with a pre-implanted jugular vein cannula (JVC) for blood sampling. A solution (5 mg/mL, free base equivalents) of the test inhibitor, N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide mono-citrate salt, was prepared freshly in 35% hydroxypropyl-β-cyclodextrin (HPBCD). Intravenous bolus injection was conducted using a 25 gauge needle via the tail vein. Animals were supplied with water and a commercial rodent diet ad libitum prior to and during the study.

Blood samples (˜100 μl at each time point) for plasma pharmacokinetic (PK) analysis were collected from the JVC cannula into lithium-heparin tubes at 2 min, 5 min, 15 min, 30 min, 45 min, 1 hr, 2 hr, 4 hr, 6 hr and 24 hr post-dose (Group A in Table 2). For compartmental PK analysis, blood and brain were collected from animals in Group B (Table 2) at 15 min, 1 hr, 2 hr, 4 hr, and 6 hr post-dose, with three rats assigned to each time point. Animals were deeply anesthetized with isoflurane and blood collected by cardiocentesis. Plasma separated by centrifugation at 6000 rpm×10 min. Brain hemispheres were subsequently harvested and snap frozen in liquid nitrogen. Plasma and brain samples were subsequently stored at −70° C.

TABLE 2
Three compartment PK Study Design

| | | | | Dosing | | | |
|---|---|---|---|---|---|---|---|
| | | | | Solution | Dosing | | |
| | Dosing | | Dose | Conc. | Volume | | Sampling Time |
| Group | Route | Animals N | mg/kg | mg/mL | mL/kg | Vehicle | Points^{a} |
| A | IV | 3 | 10 | 5 | 2 | 35% | 2, 5, 15, 30, 45 |
| | | (for PK) | | | | HPβCD | minutes, 1, 2, 4, 6, |
| | | | | | | | and 24 hours |
| | | | | | | | postdose |
| B | IV | 15 (n = 3 | 10 | 5 | 2 | 35% | 15 minutes, 1, 2, 4, |
| | | per | | | | HPβCD | and 6 hours postdose |
| | | sampling | | | | | |
| | | time) | | | | | |

^{a}Group A: animals for plasma PK sacrificed at 24 hours post-administration. Group B: animals for brain analysis sacrificed at each sampling time point (n = 3 per time point).

Bioanalysis of the test inhibitor from plasma and brain extracts was performed according to standard procedures. Briefly, 20 μl of plasma was extracted with 100 μl of acetonitrile using a 96-well Solvinert filter plate (Millipore). Samples were subjected to LC/MS/MS analysis using an Eksigent Express LC 100 microflow HPLC system, coupled with an Eksigent CXP C-18AR 0.3*50 mm column, and a Thermo TSQuantum Classic triple quadrupole mass spectrometer running in positive ion, SRM mode. Concentration of the test inhibitor was determined using a standard curve prepared in pre-extracted control plasma.

Pharmacokinetic analysis was performed using the validated pharmacokinetic program WinNonlin Professional version 5.2 software (Pharsight Co., Mountain View, Calif.). Plasma test inhibitor concentration versus time course data was processed using the noncompartmental analysis method (NCA Model 201 for bolus intravenous infusion). Brain test inhibitor concentration versus time course data was processed using the NCA Model 200 (extravascular model). Detailed pharmacokinetic data is included in Tables 3-4.

TABLE 3
Plasma and Brain AUC analysis of single dose
i.v. test inhibitor at 10 mg/kg in rats

| Compartment | AUC24 hr (h*ng/ml) | Penetration (%)^{a} |
|---|---|---|
| Plasma^{b} | 2579.9 | — |
| Brain^{b} | 1206.2 | 46.8 |

^{a}Penetration percentage relative to plasma test inhibitor AUC24 hr.
^{b}Plasma and brain sampled from the same animals at each sampling time point.

TABLE 4
Plasma and Brain concentrations (ng/mL) and pharmacokinetic
parameters for a test inhibitor following intravenous
bolus administration at 10 mg/kg in rats

| | | |
|---|---|---|
| Time (hr) | Mean | SD |

Plasma

| | | |
|---|---|---|
| 0.25 | 2497.4 | 457.5 |
| 1 | 547.6 | 157.1 |
| 2 | 130.3 | 20.3 |
| 4 | 31.1 | 5.4 |
| 6 | 5.7 | 1.6 |
| 24 | 2.2 | 0.7 |
| AUC_{24 hr} | 2579.9 | |
| (hr*ng/mL) | | |
| Terminal | 13.1 | |
| phase t_{1/2} | | |
| (hr) | | |

Brain

| | | |
|---|---|---|
| 0.25 | 159.6 | 15.0 |
| 1 | 71.1 | 26.1 |
| 2 | 72.4 | 75.9 |
| 4 | 28.0 | 12.7 |
| 6 | 42.7 | 11.3 |
| 24 | 52.5 | 12.5 |
| AUC_{24 hr} | 1206.2 | |
| (hr*ng/mL) | | |
| Terminal | 34.3 | |
| phase t_{1/2} | | |
| (hr) | | |
| MRTₗₐₛₜ | 12.0 | |
| (hr) | | |

Composite concentration time course relations were generated, with three rats assigned to each time point. Plasma and brain were sampled from the same set of animals per time point. The means (shown above) were used for WinNonlin analysis. AUC_{24 hr}: Area under the curve, calculated to the 24 hour timepoint; MRTₗₐₛₜ: Mean residence time, calculated to the last time point.

The above study evaluated the plasma pharmacokinetic profile and brain penetration of the test inhibitor, N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide, following a single 10 mg/kg intravenous dose in rats. The plasma and brain drug concentration time course relations were analyzed. The plasma and brain samples were collected from the same animals at each sampling time point, thereby reflecting a true compartmental distribution relationship during a 24 hr period post inhibitor administration. The 24 hr plasma AUC measured from this experiment was 2580 ng/mL*hr. The test inhibitor administered by intravenous injection exhibited excellent brain penetration. The brain-plasma ratio of 24 hr AUC's revealed an overall 47% penetration in brain.

The test inhibitor rapidly penetrated the CNS. The maximum concentration of the test inhibitor measured from the brain was 160 ng/mL at 15 min (the first time point measured). After 4 hrs post-dose, the brain test inhibitor level was higher than in plasma and remained significantly higher (52.5 vs. 2.2 ng/mL) at 24 hrs. This finding indicates that the clearance kinetics in brain is slower than what is observed in the peripheral compartment. The level of the test inhibitor was sustained in the brain as reflected by the long half-life (34 hrs), which was much higher than that in plasma (6-13 hrs).

1. A compound which is:
N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-4-((3-methoxybenzyl)(methyl)amino)-1-phenylbutan-2-yl)-N3-methyl-N3-((4-methylthiazol-2-yl)methyl)isophthalamide;
N1-((2S,3R)-4-(3-(dimethylamino)benzylamino)-3-hydroxy-1-phenylbutan-2-yl)-N3-methyl-N3-((4-methylthiazol-2-yl)methyl)isophthalamide;
N1-((2S,3R)-4-(3-fluoro-5-(trifluoromethyl)benzylamino)-3-hydroxy-1-phenylbutan-2-yl)-N3-methyl-N3-((4-methylthiazol-2-yl)methyl)isophthalamide;
N1-((2S,3R)-4-(2-fluoro-6-methoxybenzylamino)-3-hydroxy-1-phenylbutan-2-yl)-N3-methyl-N3-((4-methylthiazol-2-yl)methyl)isophthalamide;
N1-((2S,3R)-4-(3-tert-butyl-2-hydroxybenzylamino)-3-hydroxy-1-phenylbutan-2-yl)-N3-methyl-N3-((4-methylthiazol-2-yl)methyl)isophthalamide;
N1-((2S,3R)-4-(5-tert-butyl-2-hydroxybenzylamino)-3-hydroxy-1-phenylbutan-2-yl)-N3-methyl-N3-((4-methylthiazol-2-yl)methyl)isophthalamide;
N1-((2S,3R)-4-(3-acetylbenzylamino)-3-hydroxy-1-phenylbutan-2-yl)-N3-methyl-N3-((4-methylthiazol-2-yl)methyl)isophthalamide;
N1-((2S,3R)-4-(3,5-dimethoxybenzylamino)-3-hydroxy-1-phenylbutan-2-yl)-N3-methyl-N3-((4-methylthiazol-2-yl)methyl)isophthalamide;
N1-((2S,3R)-4-(3,4-dimethylbenzylamino)-3-hydroxy-1-phenylbutan-2-yl)-N3-methyl-N3-((4-methylthiazol-2-yl)methyl)isophthalamide;
N1-((2S,3R)-4-(3,5-bis(trifluoromethyl)benzylamino)-3-hydroxy-1-phenylbutan-2-yl)-N3-methyl-N3-((4-methylthiazol-2-yl)methyl)isophthalamide;
methyl 3-(((2R,3S)-2-hydroxy-3-(3-(methyl((4-methylthiazol-2-yl)methyl)carbamoyl)benzamido)-4-phenylbutylamino)methyl)-5-methoxybenzoate;
3-(((2R,3S)-2-hydroxy-3-(3-(methyl((4-methylthiazol-2-yl)methyl)carbamoyl)benzamido)-4-phenylbutylamino)methyl)-5-methoxybenzoic acid;
N1-((2S,3R)-3-hydroxy-4-(3-iodobenzylamino)-1-phenylbutan-2-yl)-N3-methyl-N3-((4-methylthiazol-2-yl)methyl)isophthalamide;
N1-((2S,3R)-4-(3-chlorobenzylamino)-3-hydroxy-1-phenylbutan-2-yl)-N3-methyl-N3-((4-methylthiazol-2-yl)methyl)isophthalamide;
N1-((2S,3R)-3-hydroxy-4-(3-methoxy-5-(methylcarbamoyl)benzylamino)-1-phenylbutan-2-yl)-N3-methyl-N3-((4-methylthiazol-2-yl)methyl)isophthalamide;
benzyl 3-(((2R,3S)-2-hydroxy-3-(3-(methyl((4-methylthiazol-2-yl)methyl)carbamoyl)benzamido)-4-phenylbutylamino)methyl)-5-isopropylphenylcarbamate;
N1-((2S,3R)-4-(3-amino-5-isopropylbenzylamino)-3-hydroxy-1-phenylbutan-2-yl)-N3-methyl-N3-((4-methylthiazol-2-yl)methyl)isophthalamide;
N1-((2S,3R)-4-(3-(dimethylamino)-5-isopropylbenzylamino)-3-hydroxy-1-phenylbutan-2-yl)-N3-methyl-N3-((4-methylthiazol-2-yl)methyl)isophthalamide;
N1-((2S,3R)-4-(3-(benzyloxy)-5-isopropylbenzylamino)-3-hydroxy-1-phenylbutan-2-yl)-N3-methyl-N3-((4-methylthiazol-2-yl)methyl)isophthalamide;
N1-((2S,3R)-3-hydroxy-4-(3-isopropyl-5-(methylsulfonamido)benzylamino)-1-phenylbutan-2-yl)-N3-methyl-N3-((4-methylthiazol-2-yl)methyl)isophthalamide;
N1-((2S,3R)-3-hydroxy-4-(3-hydroxy-5-isopropylbenzylamino)-1-phenylbutan-2-yl)-N3-methyl-N3-((4-methylthiazol-2-yl)methyl)isophthalamide;
benzyl 3-(((2R,3S)-2-hydroxy-3-(3-(methyl((4-methylthiazol-2-yl)methyl)carbamoyl)benzamido)-4-phenylbutylamino)methyl)-5-isopropylphenyl(methyl)carbamate;
N1-((2S,3R)-3-hydroxy-4-(3-isopropyl-5-(methylamino)benzylamino)-1-phenylbutan-2-yl)-N3-methyl-N3-((4-methylthiazol-2-yl)methyl)isophthalamide;
methyl 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoate;
N1-((2S,3R)-3-hydroxy-4-(3-hydroxybenzylamino)-1-phenylbutan-2-yl)-N3-methyl-N3-((4-methylthiazol-2-yl)methyl)isophthalamide;
N1-((2S,3R)-4-(3-tert-butylbenzylamino)-3-hydroxy-1-phenylbutan-2-yl)-N3-methyl-N3-((4-methylthiazol-2-yl)methyl)isophthalamide;
N1-((2S,3R)-4-(3-cyclopropylbenzylamino)-3-hydroxy-1-phenylbutan-2-yl)-N3-methyl-N3-((4-methylthiazol-2-yl)methyl)isophthalamide;
N1-((2S,3R)-3-hydroxy-4-(naphthalen-1-ylmethylamino)-1-phenylbutan-2-yl)-N3-methyl-N3-((4-methylthiazol-2-yl)methyl)isophthalamide;
N1-((2S,3R)-3-hydroxy-1-phenyl-4-(pyridin-3-ylmethylamino)butan-2-yl)-N3-methyl-N3-((4-methylthiazol-2-yl)methyl)isophthalamide;
N1-((2S,3R)-3-hydroxy-1-phenyl-4-(pyridin-4-ylmethylamino)butan-2-yl)-N3-methyl-N3-((4-methylthiazol-2-yl)methyl)isophthalamide;
N1-((2S,3R)-3-hydroxy-1-phenyl-4-(pyridin-2-ylmethylamino)butan-2-yl)-N3-methyl-N3-((4-methylthiazol-2-yl)methyl)isophthalamide;
N1-((2S,3R)-3-hydroxy-1-phenyl-4-(1-(pyridin-3-yl)ethylamino)butan-2-yl)-N3-methyl-N3-((4-methylthiazol-2-yl)methyl)isophthalamide;
N1-((2S,3R)-3-hydroxy-4-((6-methylpyridin-2-yl)methylamino)-1-phenylbutan-2-yl)-N3-methyl-N3-((4-methylthiazol-2-yl)methyl)isophthalamide;
N1-((2R,3S)-3-hydroxy-4-((5-isopropylpyridin-3-yl)methylamino)-1-phenylbutan-2-yl)-N3-methyl-N3-((4-methylthiazol-2-yl)methyl)isophthalamide;
N1-((2S,3R)-3-hydroxy-1-phenyl-4-((6-(trifluoromethyl)pyridin-3-yl)methylamino)butan-2-yl)-N3-methyl-N3-((4-methylthiazol-2-yl)methyl)isophthalamide;
N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide;
N1-((2S,3R)-3-hydroxy-4-((4-isopropylpyridin-2-yl)methylamino)-1-phenylbutan-2-yl)-N3-methyl-N3-((4-methylthiazol-2-yl)methyl)isophthalamide;
N1-((2S,3R)-3-hydroxy-4-((6-isopropylpyridin-2-yl)methylamino)-1-phenylbutan-2-yl)-N3-methyl-N3-((4-methylthiazol-2-yl)methyl)isophthalamide;
N1-((2S,3R)-3-hydroxy-4-((2-isopropylpyridin-4-yl)methylamino)-1-phenylbutan-2-yl)-N3-methyl-N3-((4-methylthiazol-2-yl)methyl)isophthalamide;
methyl 6-(((2R,3S)-2-hydroxy-3-(3-(methyl((4-methylthiazol-2-yl)methyl)carbamoyl)benzamido)-4-phenylbutylamino)methyl)-2-methylnicotinate;
N1-((2S,3R)-3-hydroxy-4-((5-(hydroxymethyl)-6-methylpyridin-2-yl)methylamino)-1-phenylbutan-2-yl)-N3-methyl-N3-((4-methylthiazol-2-yl)methyl)isophthalamide;
N1-((2S,3R)-4-((5-(dimethylamino)pyridin-3-yl)methylamino)-3-hydroxy-1-phenylbutan-2-yl)-N3-methyl-N3-((4-methylthiazol-2-yl)methyl)isophthalamide;
N1-((2S,3R)-4-((2-fluoro-5-isopropylpyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-4-((5-isopentylpyridin-3-yl)methylamino)-1-phenylbutan-2-yl)-N3-methyl-N3-((4-methylthiazol-2-yl)methyl)isophthalamide;
N1-((2S,3R)-3-hydroxy-4-((6-methyl-5-(methylthiomethyl)pyridin-2-yl)methylamino)-1-phenylbutan-2-yl)-N3-methyl-N3-((4-methylthiazol-2-yl)methyl)isophthalamide;
N1-((2S,3R)-4-((2,6-diisopropylpyridin-4-yl)methylamino)-3-hydroxy-1-phenylbutan-2-yl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-N3-methyl-N3-((R)-1-(4-methylthiazol-2-yl)ethyl)isophthalamide;
N1-((2S,3R)-4-((5-cyclopropylpyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-1-phenyl-4-((5-(prop-1-en-2-yl)pyridin-3-yl)methylamino)butan-2-yl)-N3-methyl-N3-((4-methylthiazol-2-yl)methyl)isophthalamide;
N1-((2S,3R)-4-((1-tert-butyl-5-methyl-1H-pyrazol-4-yl)methylamino)-3-hydroxy-1-phenylbutan-2-yl)-N3-methyl-N3-((4-methylthiazol-2-yl)methyl)isophthalamide;
N1-((2S,3R)-3-hydroxy-4-((1-(2-methoxyethyl)-5-methyl-1H-pyrazol-4-yl)methylamino)-1-phenylbutan-2-yl)-N3-methyl-N3-((4-methylthiazol-2-yl)methyl)isophthalamide;
N1-((2S,3R)-3-hydroxy-4-((4-methoxypyrimidin-2-yl)methylamino)-1-phenylbutan-2-yl)-N3-methyl-N3-((4-methylthiazol-2-yl)methyl)isophthalamide;
N1-((2S,3R)-4-((1H-indol-7-yl)methylamino)-3-hydroxy-1-phenylbutan-2-yl)-N3-methyl-N3-((4-methylthiazol-2-yl)methyl)isophthalamide;
N1-cyclopropyl-N3-((2S,3R)-3-hydroxy-1-phenyl-4-(3-(trifluoromethyl)benzylamino)butan-2-yl)-N1-((4-methylthiazol-2-yl)methyl)isophthalamide;
N1-cyclopropyl-N3-((2S,3R)-3-hydroxy-1-phenyl-4-(3-(trifluoromethoxy)benzylamino)butan-2-yl)-N1-((4-methylthiazol-2-yl)methyl)isophthalamide;
N1-((2S,3R)-3-hydroxy-4-(3-methoxybenzylamino)-1-phenylbutan-2-yl)-N3-((4-methylthiazol-2-yl)methyl)-N3-propylisophthalamide;
N1-cyclopropyl-N3-((2S,3R)-3-hydroxy-1-phenyl-4-((5-(trifluoromethyl)pyridin-3-yl)methylamino)butan-2-yl)-N1-((4-methylthiazol-2-yl)methyl)isophthalamide;
N1-((2S,3R)-3-hydroxy-4-((5-isopropyl-2,6-dimethylpyridin-3-yl)methylamino)-1-phenylbutan-2-yl)-N3-((4-methylthiazol-2-yl)methyl)isophthalamide;
N1-((2S,3R)-3-hydroxy-4-((6-methyl-5-(methylsulfonylmethyl)pyridin-2-yl)methylamino)-1-phenylbutan-2-yl)-N3-((4-methylthiazol-2-yl)methyl)isophthalamide;
N1-((4,5-dimethylthiazol-2-yl)methyl)-N3-((2S,3R)-3-hydroxy-4-(3-methoxybenzylamino)-1-phenylbutan-2-yl)-N1-methylisophthalamide;
N1-((2S,3R)-3-hydroxy-4-(3-methoxybenzylamino)-1-phenylbutan-2-yl)-N3-methyl-N3-((5-methylthiazol-2-yl)methyl)isophthalamide;
N1-((2S,3R)-3-hydroxy-4-(3-methoxybenzylamino)-1-phenylbutan-2-yl)-N3-(4-methylthiazol-2-yl)isophthalamide;
N1-((2S,3R)-3-hydroxy-4-(3-methoxybenzylamino)-1-phenylbutan-2-yl)-N3-methyl-N3-(4-methylthiazol-2-yl)isophthalamide;
N1-((2S,3R)-3-hydroxy-4-(3-methoxybenzylamino)-1-phenylbutan-2-yl)-N3-methyl-N3-((1-methyl-1H-pyrazol-3-yl)methyl)isophthalamide;
N1-(furan-2-ylmethyl)-N3-((2S,3R)-3-hydroxy-4-(3-methoxybenzylamino)-1-phenylbutan-2-yl)-N1-methylisophthalamide;
N1-((2S,3R)-3-hydroxy-4-(3-methoxybenzylamino)-1-phenylbutan-2-yl)-N3-methyl-N3-((5-methylfuran-2-yl)methyl)isophthalamide;
N1-((2S,3R)-3-hydroxy-4-((5-isopropylpyridin-3-yl)methylamino)-1-phenylbutan-2-yl)-N3-methyl-N3-((4-methyloxazol-2-yl)methyl)isophthalamide;
N1-((2S,3R)-3-hydroxy-4-(3-methoxybenzylamino)-1-phenylbutan-2-yl)-N3-methyl-N3-(pyridin-2-ylmethyl)isophthalamide;
N1-((2S,3R)-3-hydroxy-4-(3-methoxybenzylamino)-1-phenylbutan-2-yl)-N3-methyl-N3-(pyridin-3-ylmethyl)isophthalamide;
N1-((2S,3R)-3-hydroxy-4-(3-methoxybenzylamino)-1-phenylbutan-2-yl)-N3-methyl-N3-((6-methylpyridin-2-yl)methyl)isophthalamide;
N1-((2S,3R)-3-hydroxy-4-(3-methoxybenzylamino)-1-phenylbutan-2-yl)-N3-((4-methylpyrimidin-2-yl)methyl)isophthalamide;
5-fluoro-N1-((2S,3R)-3-hydroxy-4-(3-methoxybenzylamino)-1-phenylbutan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-N3,5-dimethyl-N3-((4-methylthiazol-2-yl)methyl)isophthalamide;
N3-((2S,3R)-3-hydroxy-4-(3-methoxybenzylamino)-1-phenylbutan-2-yl)-N1,4-dimethyl-N1-(4-methylthiazol-2-yl)methyl)isophthalamide;
N1-((2S,3R)-3-hydroxy-4-(3-methoxybenzylamino)-1-phenylbutan-2-yl)-N3,4-dimethyl-N3-((4-methylthiazol-2-yl)methyl)isophthalamide;
5-tert-butyl-N1-((2S,3R)-3-hydroxy-4-(3-methoxybenzylamino)-1-phenylbutan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-N3-methyl-N3-((4-methylthiazol-2-yl)methyl)-5-vinylisophthalamide;
N1-cyclopropyl-N3-((2S,3R)-3-hydroxy-4-(3-methoxybenzylamino)-1-phenylbutan-2-yl)-5-methyl-N1-((4-methylthiazol-2-yl)methyl)isophthalamide;
N1-((2S,3R)-3-hydroxy-4-((5-isopropylpyridin-3-yl)methylamino)-1-phenylbutan-2-yl)-N3,5-dimethyl-N3-((4-methylthiazol-2-yl)methyl)isophthalamide;
N1-((2S,3R)-3-hydroxy-4-((2-isopropylpyridin-4-yl)methylamino)-1-phenylbutan-2-yl)-N3,5-dimethyl-N3-((4-methylthiazol-2-yl)methyl)isophthalamide;
N1-cyclopropyl-N3-((2S,3R)-3-hydroxy-4-((5-isopropylpyridin-3-yl)methylamino)-1-phenylbutan-2-yl)-5-methyl-N1-((4-methylthiazol-2-yl)methyl)isophthalamide;
5-hydroxy-N1-((2S,3R)-3-hydroxy-4-(3-methoxybenzylamino)-1-phenylbutan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-5-methoxy-N3-methyl-N3-((4-methylthiazol-2-yl)methyl)isophthalamide;
N1-((2S,3R)-3-hydroxy-1-phenyl-4-(3-(trifluoromethyl)benzylamino)butan-2-yl)-5-methoxy-N3-methyl-N3-((4-methylthiazol-2-yl)methyl)isophthalamide;
N1-ethyl-N3-((2S,3R)-3-hydroxy-4-(3-methoxybenzylamino)-1-phenylbutan-2-yl)-5-methoxy-N1-((4-methylthiazol-2-yl)methyl)isophthalamide;
N1-((2S,3R)-3-hydroxy-4-(3-methoxybenzylamino)-1-phenylbutan-2-yl)-5-methoxy-N3-((4-methylthiazol-2-yl)methyl)-N3-propylisophthalamide;
N1-((2S,3R)-3-hydroxy-4-(3-methoxybenzylamino)-1-phenylbutan-2-yl)-N3-isopropyl-5-methoxy-N3-((4-methylthiazol-2-yl)methyl)isophthalamide;
N1-((2S,3R)-3-hydroxy-4-(3-methoxybenzylamino)-1-phenylbutan-2-yl)-N3-(2-hydroxyethyl)-5-methoxy-N3-((4-methylthiazol-2-yl)methyl)isophthalamide;
N1-((2S,3R)-3-hydroxy-4-((2-isopropylpyridin-4-yl)methylamino)-1-phenylbutan-2-yl)-5-methoxy-N3-methyl-N3-((4-methylthiazol-2-yl)methyl)isophthalamide;
N1-((2S,3R)-3-hydroxy-4-((2-isopropylpyridin-4-yl)methylamino)-1-phenylbutan-2-yl)-5-methoxy-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-5-methoxy-N3-methyl-N3-((4-methylthiazol-2-yl)methyl)isophthalamide;
N1-((2S,3R)-3-hydroxy-4-(3-methoxybenzylamino)-1-phenylbutan-2-yl)-5-methoxy-N3-((R)-1-phenylethyl)isophthalamide;
N1-((2S,3R)-3-hydroxy-4-(3-methoxybenzylamino)-1-phenylbutan-2-yl)-5-(hydroxymethyl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-5-(methoxymethyl)-N3-methyl-N3-((4-methylthiazol-2-yl)methyl)isophthalamide;
3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzoic acid;
methyl 3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzoate;
N1-((2S,3R)-3-hydroxy-4-(3-methoxybenzylamino)-1-phenylbutan-2-yl)-N3-methyl-N3-((4-methylthiazol-2-yl)methyl)benzene-1,3,5-tricarboxamide;
N1-((2S,3R)-3-hydroxy-4-(3-methoxybenzylamino)-1-phenylbutan-2-yl)-N3,N5-dimethyl-N3-((4-methylthiazol-2-yl)methyl)benzene-1,3,5-tricarboxamide;
N1-((2S,3R)-3-hydroxy-4-(3-methoxybenzylamino)-1-phenylbutan-2-yl)-N3-(2-hydroxyethyl)-N5-methyl-N5-((4-methylthiazol-2-yl)methyl)benzene-1,3,5-tricarboxamide;
(S)-methyl 3-hydroxy-2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)benzamido)propanoate;
N1-((2S,3R)-3-hydroxy-4-(3-methoxybenzylamino)-1-phenylbutan-2-yl)-N3-((R)-1-phenylethyl)benzene-1,3,5-tricarboxamide;
N1-((2S,3R)-3-hydroxy-4-(3-methoxybenzylamino)-1-phenylbutan-2-yl)-N3-methyl-N5-((R)-1-phenylethyl)benzene-1,3,5-tricarboxamide;
5-(4,5-dihydrooxazol-2-yl)-N1-((2S,3R)-3-hydroxy-4-(3-methoxybenzylamino)-1-phenylbutan-2-yl)-N3-methyl-N3-((4-methylthiazol-2-yl)methyl)isophthalamide;
(S)-methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)-4,5-dihydrooxazole-4-carboxylate;
N1-((2S,3R)-3-hydroxy-4-(3-methoxybenzylamino)-1-phenylbutan-2-yl)-N3-methyl-N3-((4-methylthiazol-2-yl)methyl)-5-(oxazol-2-yl)isophthalamide;
methyl 2-(3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl)oxazole-4-carboxylate;
N3-((2S,3R)-3-hydroxy-4-(3-methoxybenzylamino)-1-phenylbutan-2-yl)-N5-methyl-N5-((4-methylthiazol-2-yl)methyl)biphenyl-3,5-dicarboxamide;
5-(azidomethyl)-N1-((2S,3R)-3-hydroxy-4-(3-methoxybenzylamino)-1-phenylbutan-2-yl)-N3-methyl-N3-((4-methylthiazol-2-yl)methyl)isophthalamide;
5-amino-N1-((2S,3R)-3-hydroxy-4-(3-methoxybenzylamino)-1-phenylbutan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-N3-methyl-5-(methylamino)-N3-((4-methylthiazol-2-yl)methyl)isophthalamide;
5-(dimethylamino)-N1-((2S,3R)-3-hydroxy-4-(3-methoxybenzylamino)-1-phenylbutan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-5-(hydroxyamino)-N3-methyl-N3-((4-methylthiazol-2-yl)methyl)isophthalamide;
N1-((2S,3R)-3-hydroxy-4-(3-methoxybenzylamino)-1-phenylbutan-2-yl)-5-(hydroxyamino)-N3-((R)-1-phenylethyl)isophthalamide;
3-((2S,3R)-3-hydroxy-4-(3-methoxybenzylamino)-1-phenylbutan-2-ylcarbamoyl)-5-(methyl((4-methylthiazol-2-yl)methyl)carbamoyl)phenyl methanesulfonate;
3-((2S,3R)-3-hydroxy-4-((5-isopropylpyridin-3-yl)methylamino)-1-phenylbutan-2-ylcarbamoyl)-5-(methyl((4-methylthiazol-2-yl)methyl)carbamoyl)phenyl methanesulfonate;
3-((2S,3R)-3-hydroxy-4-(3-methoxybenzylamino)-1-phenylbutan-2-ylcarbamoyl)-5-((R)-1-phenylethylcarbamoyl)phenyl methanesulfonate;
N1-((2S,3R)-3-hydroxy-4-(3-hydroxybenzylamino)-1-phenylbutan-2-yl)-5-(N-methylmethylsulfonamido)-N3-((R)-1-phenylethyl)isophthalamide;
N1-((2S,3R)-4-(3-tert-butyl-2-hydroxybenzylamino)-3-hydroxy-1-phenylbutan-2-yl)-5-(N-methylmethylsulfonamido)-N3-((R)-1-phenylethyl)isophthalamide;
N1-((2S,3R)-4-(5-tert-butyl-2-(tert-butyldimethylsilyloxy)benzylamino)-3-hydroxy-1-phenylbutan-2-yl)-5-(N-methylmethylsulfonamido)-N3-((R)-1-phenylethyl)isophthalamide;
N1-((2S,3R)-4-(5-tert-butyl-2-hydroxybenzylamino)-3-hydroxy-1-phenylbutan-2-yl)-5-(N-methylmethylsulfonamido)-N3-((R)-1-phenylethyl)isophthalamide;
N1-((2S,3R)-4-(3-acetylbenzylamino)-3-hydroxy-1-phenylbutan-2-yl)-5-(N-methylmethylsulfonamido)-N3-((R)-1-phenylethyl)isophthalamide;
N1-((2S,3R)-4-(3-chlorobenzylamino)-3-hydroxy-1-phenylbutan-2-yl)-5-(N-methylmethylsulfonamido)-N3-((R)-1-phenylethyl)isophthalamide;
N1-((2S,3R)-3-hydroxy-4-(3-iodobenzylamino)-1-phenylbutan-2-yl)-5-(N-methylmethylsulfonamido)-N3-((R)-1-phenylethyl)isophthalamide;
methyl 3-(((2R,3S)-2-hydroxy-3-(3-(N-methylmethylsulfonamido)-5-((R)-1-phenylethylcarbamoyl)benzamido)-4-phenylbutylamino)methyl)benzoate;
N1-((2S,3R)-3-hydroxy-4-(3-(hydroxymethyl)benzylamino)-1-phenylbutan-2-yl)-5-(N-methylmethylsulfonamido)-N3-((R)-1-phenylethyl)isophthalamide;
3-(((2R,3S)-2-hydroxy-3-(3-(N-methylmethylsulfonamido)-5-((R)-1-phenylethylcarbamoyl)benzamido)-4-phenylbutylamino)methyl)benzoic acid;
methyl 3-(((2R,3S)-2-hydroxy-3-(3-(N-methylmethylsulfonamido)-5-((R)-1-phenylethylcarbamoyl)benzamido)-4-phenylbutylamino)methyl)-5-methoxybenzoate;
N1-((2S,3R)-4-(3-tert-butylbenzylamino)-3-hydroxy-1-phenylbutan-2-yl)-5-(N-methylmethylsulfonamido)-N3-((R)-1-phenylethyl)isophthalamide;
N1-((2S,3R)-4-((5-(2-fluoropropan-2-yl)pyridin-3-yl)methylamino)-3-hydroxy-1-phenylbutan-2-yl)-5-(N-methylmethylsulfonamido)-N3-((R)-1-phenylethyl)isophthalamide;
N1-((2S,3R)-3-hydroxy-1-phenyl-4-((5-(trifluoromethyl)pyridin-3-yl)methylamino)butan-2-yl)-5-(N-methylmethylsulfonamido)-N3-((R)-1-phenylethyl)isophthalamide;
N1-((2S,3R)-4-((1-tert-butyl-5-methyl-1H-pyrazol-4-yl)methylamino)-3-hydroxy-1-phenylbutan-2-yl)-5-(N-methylmethylsulfonamido)-N3-((R)-1-phenylethyl)isophthalamide;
N1-((2S,3R)-3-hydroxy-4-((1-(2-methoxyethyl)-5-methyl-1H-pyrazol-4-yl)methylamino)-1-phenylbutan-2-yl)-5-(N-methylmethylsulfonamido)-N3-((R)-1-phenylethyl)isophthalamide;
N1-((2S,3R)-3-hydroxy-4-(3-methoxybenzylamino)-1-phenylbutan-2-yl)-5-(N-methylmethylsulfonamido)-N3-((6-methylpyridin-2-yl)methyl)isophthalamide;
N1-((2S,3R)-3-hydroxy-4-(3-methoxybenzylamino)-1-phenylbutan-2-yl)-5-(N-methylmethylsulfonamido)-N3-((4-methylpyrimidin-2-yl)methyl)isophthalamide;
N1-((2S,3R)-3-hydroxy-1-phenyl-4-((S)-1-phenylethylamino)butan-2-yl)-5-(N-methylmethylsulfonamido)-N3-(1-(5-methylthiophen-2-yl)ethyl)isophthalamide;
N1-((2S,3R)-3-hydroxy-4-(methyl((S)-1-phenylethyl)amino)-1-phenylbutan-2-yl)-N3-((R)-1-(3-methoxyphenyl)ethyl)-5-(N-methylmethylsulfonamido)isophthalamide;
N1-((2S,3R)-3-hydroxy-4-(methyl((S)-1-phenylethyl)amino)-1-phenylbutan-2-yl)-N3-(1-(5-methylfuran-2-yl)ethyl)-5-(N-methylmethylsulfonamido)isophthalamide;
N1-((2S,3R)-3-hydroxy-4-(methyl((S)-1-phenylethyl)amino)-1-phenylbutan-2-yl)-5-(N-methylmethylsulfonamido)-N3-(1-(5-methylthiophen-2-yl)ethyl)isophthalamide;
N1-((2S,3R)-3-hydroxy-4-(methyl((S)-1-phenylethyl)amino)-1-phenylbutan-2-yl)-5-(N-methylmethylsulfonamido)-N3-(1-(3-methylthiophen-2-yl)ethyl)isophthalamide;
N1-((2S,3R)-3-hydroxy-1-phenyl-4-((S)-1-phenylethylamino)butan-2-yl)-N3-((S)-1-phenylethyl)isophthalamide;
N1-((2S,3R)-3-hydroxy-1-phenyl-4-((S)-1-phenylethylamino)butan-2-yl)-N3-((R)-1-p-tolylethyl)isophthalamide;
N1-((2S,3R)-3-hydroxy-1-phenyl-4-((S)-1-phenylethylamino)butan-2-yl)-N3-((R)-1-(4-methoxyphenyl)ethyl)isophthalamide;
N1-((2S,3R)-3-hydroxy-1-phenyl-4-((S)-1-phenylethylamino)butan-2-yl)-N3-((R)-1-(3-methoxyphenyl)ethyl)isophthalamide;
N1-((2S,3R)-3-hydroxy-1-phenyl-4-((S)-1-phenylethylamino)butan-2-yl)-N3-(1-o-tolylethyl)isophthalamide;
N1-((2S,3R)-3-hydroxy-4-(methyl((S)-1-phenylethyl)amino)-1-phenylbutan-2-yl)-N3-(1-(3-methylthiophen-2-yl)ethyl)isophthalamide;
N1-((2S,3R)-3-hydroxy-1-phenyl-4-((R)-1-p-tolylethylamino)butan-2-yl)-N3-((R)-1-p-tolylethyl)isophthalamide;
N1-((2S,3R)-3-hydroxy-1-phenyl-4-((R)-1-p-tolylethylamino)butan-2-yl)-N3-((R)-1-(4-methoxyphenyl)ethyl)isophthalamide;
N1-((2S,3R)-3-hydroxy-4-((R)-1-(4-methoxyphenyl)ethylamino)-1-phenylbutan-2-yl)-N3-((R)-1-p-tolylethyl)isophthalamide;
N1-((2S,3R)-3-hydroxy-4-((R)-1-(4-methoxyphenyl)ethylamino)-1-phenylbutan-2-yl)-N3-((R)-1-(4-methoxyphenyl)ethyl)isophthalamide;
N1-((2S,3R)-3-hydroxy-1-phenyl-4-((S)-1-phenylethylamino)butan-2-yl)-5-methyl-N3-((S)-1-phenylethyl)isophthalamide;
N1-((2S,3R)-3-hydroxy-1-phenyl-4-((S)-1-phenylethylamino)butan-2-yl)-5-methyl-N3-(1-(5-methylfuran-2-yl)ethyl)isophthalamide;
N1-((2S,3R)-3-hydroxy-1-phenyl-4-((S)-1-phenylethylamino)butan-2-yl)-5-methyl-N3-(1-o-tolylethyl)isophthalamide;
N1-((2S,3R)-3-hydroxy-4-(methyl((S)-1-phenylethyl)amino)-1-phenylbutan-2-yl)-5-methyl-N3-((R)-1-p-tolylethyl)isophthalamide;
N1-((2S,3R)-3-hydroxy-4-(methyl((S)-1-phenylethyl)amino)-1-phenylbutan-2-yl)-N3-((R)-1-(4-methoxyphenyl)ethyl)-5-methylisophthalamide;
N1-((2S,3R)-3-hydroxy-4-(methyl((S)-1-phenylethyl)amino)-1-phenylbutan-2-yl)-N3-((R)-1-(3-methoxyphenyl)ethyl)-5-methylisophthalamide;
N1-((2S,3R)-3-hydroxy-4-(methyl((S)-1-phenylethyl)amino)-1-phenylbutan-2-yl)-5-methyl-N3-(1-(5-methylthiophen-2-yl)ethyl)isophthalamide;
N1-((2S,3R)-3-hydroxy-1-phenyl-4-((R)-1-p-tolylethylamino)butan-2-yl)-5-methyl-N3-((R)-1-p-tolylethyl)isophthalamide;
N1-((2S,3R)-3-hydroxy-1-phenyl-4-((R)-1-p-tolylethylamino)butan-2-yl)-5-methyl-N3-(1-o-tolylethyl)isophthalamide;
N1-((2S,3R)-3-hydroxy-4-((R)-1-(3-methoxyphenyl)ethylamino)-1-phenylbutan-2-yl)-5-methyl-N3-(1-(pyrazin-2-yl)ethyl)isophthalamide;
N1-((2S,3R)-3-hydroxy-1-phenyl-4-((S)-1-phenylethylamino)butan-2-yl)-N3-methyl-5-(N-methylmethylsulfonamido)-N3-((S)-1-phenylethyl)isophthalamide;
N1-((2S,3R)-3-hydroxy-1-phenyl-4-((S)-1-phenylethylamino)butan-2-yl)-N3-((R)-1-(3-methoxyphenyl)ethyl)-5-(N-methylmethylsulfonamido)isophthalamide;
N1-((2S,3R)-3-hydroxy-1-phenyl-4-((S)-1-phenylethylamino)butan-2-yl)-N3-(1-(5-methylfuran-2-yl)ethyl)-5-(N-methylmethylsulfonamido)isophthalamide;
N1-((2S,3R)-3-hydroxy-1-phenyl-4-((S)-1-phenylethylamino)butan-2-yl)-5-(N-methylmethylsulfonamido)-N3-(1-o-tolylethyl)isophthalamide;
N1-((2S,3R)-3-hydroxy-1-phenyl-4-((R)-1-p-tolylethylamino)butan-2-yl)-5-(N-methylmethylsulfonamido)-N3-((S)-1-phenylethyl)isophthalamide;
N1-((2S,3R)-3-hydroxy-1-phenyl-4-((R)-1-p-tolylethylamino)butan-2-yl)-N3-((R)-1-(4-methoxyphenyl)ethyl)-5-(N-methylmethylsulfonamido)isophthalamide;
N1-((2S,3R)-3-hydroxy-1-phenyl-4-((R)-1-p-tolylethylamino)butan-2-yl)-N3-((R)-1-(3-methoxyphenyl)ethyl)-5-(N-methylmethylsulfonamido)isophthalamide;
N1-((2S,3R)-3-hydroxy-4-((R)-1-(4-methoxyphenyl)ethylamino)-1-phenylbutan-2-yl)-5-(N-methylmethylsulfonamido)-N3-((R)-1-p-tolylethyl)isophthalamide;
N1-((2S,3R)-3-hydroxy-4-((R)-1-(4-methoxyphenyl)ethylamino)-1-phenylbutan-2-yl)-N3-((R)-1-(4-methoxyphenyl)ethyl)-5-(N-methylmethylsulfonamido)isophthalamide;
N1-((2S,3R)-3-hydroxy-4-((R)-1-(4-methoxyphenyl)ethylamino)-1-phenylbutan-2-yl)-5-(N-methylmethylsulfonamido)-N3-(1-(5-methylthiophen-2-yl)ethyl)isophthalamide;
N1-((2S,3R)-3-hydroxy-4-((R)-1-(4-methoxyphenyl)ethylamino)-1-phenylbutan-2-yl)-5-(N-methylmethylsulfonamido)-N3-(1-(3-methylthiophen-2-yl)ethyl)isophthalamide;
N1-((2S,3R)-3-hydroxy-4-((R)-1-(3-methoxyphenyl)ethylamino)-1-phenylbutan-2-yl)-N3-methyl-5-(N-methylmethylsulfonamido)-N3-((S)-1-phenylethyl)isophthalamide;
N1-((2S,3R)-3-hydroxy-4-((R)-1-(3-methoxyphenyl)ethylamino)-1-phenylbutan-2-yl)-N3-((R)-1-(4-methoxyphenyl)ethyl)-5-(N-methylmethylsulfonamido)isophthalamide;
N1-(1-(furan-2-yl)ethyl)-N3-((2S,3R)-3-hydroxy-4-((R)-1-(3-methoxyphenyl)ethylamino)-1-phenylbutan-2-yl)-5-(N-methylmethylsulfonamido)isophthalamide;
N1-((2S,3R)-3-hydroxy-4-(methyl((S)-1-phenylethyl)amino)-1-phenylbutan-2-yl)-N3-((S)-1-phenylethyl)isophthalamide;
N1-(1-(furan-2-yl)ethyl)-N3-((2S,3R)-3-hydroxy-1-phenyl-4-((S)-1-phenylethylamino)butan-2-yl)isophthalamide;
N1-((2S,3R)-3-hydroxy-4-(methyl((S)-1-phenylethyl)amino)-1-phenylbutan-2-yl)-N3-((R)-1-p-tolylethyl)isophthalamide;
N1-((2S,3R)-3-hydroxy-4-(methyl((S)-1-phenylethyl)amino)-1-phenylbutan-2-yl)-N3-((R)-1-(4-methoxyphenyl)ethyl)isophthalamide;
N1-(1-(furan-2-yl)ethyl)-N3-((2S,3R)-3-hydroxy-4-(methyl((S)-1-phenylethyl)amino)-1-phenylbutan-2-yl)isophthalamide;
N1-((2S,3R)-3-hydroxy-1-phenyl-4-((R)-1-p-tolylethylamino)butan-2-yl)-N3-(1-o-tolylethyl)isophthalamide;
N1-((2S,3R)-3-hydroxy-4-((R)-1-(4-methoxyphenyl)ethylamino)-1-phenylbutan-2-yl)-N3-((S)-1-phenylethyl)isophthalamide;
N1-((2S,3R)-3-hydroxy-4-((R)-1-(4-methoxyphenyl)ethylamino)-1-phenylbutan-2-yl)-N3-((R)-1-(3-methoxyphenyl)ethyl)isophthalamide;
N1-(1-(2,3-dihydrofuran-2-yl)ethyl)-N3-((2S,3R)-3-hydroxy-4-((R)-1-(4-methoxyphenyl)ethylamino)-1-phenylbutan-2-yl)isophthalamide;
N1-((2S,3R)-3-hydroxy-4-((R)-1-(4-methoxyphenyl)ethylamino)-1-phenylbutan-2-yl)-N3-(1-(5-methyl-2,3-dihydrofuran-2-yl)ethyl)isophthalamide;
N1-((2S,3R)-3-hydroxy-4-((R)-1-(4-methoxyphenyl)ethylamino)-1-phenylbutan-2-yl)-N3-(1-o-tolylethyl)isophthalamide;
N1-((2S,3R)-3-hydroxy-4-((R)-1-(4-methoxyphenyl)ethylamino)-1-phenylbutan-2-yl)-N3-(1-(pyrazin-2-yl)ethyl)isophthalamide;
N1-((2S,3R)-3-hydroxy-4-((R)-1-(4-methoxyphenyl)ethylamino)-1-phenylbutan-2-yl)-N3-(1-(3-methylthiophen-2-yl)ethyl)isophthalamide;
N1-((2S,3R)-3-hydroxy-4-((R)-1-(3-methoxyphenyl)ethylamino)-1-phenylbutan-2-yl)-N3-((S)-1-phenylethyl)isophthalamide;
N1-((2S,3R)-3-hydroxy-4-((R)-1-(3-methoxyphenyl)ethylamino)-1-phenylbutan-2-yl)-N3-((R)-1-(3-methoxyphenyl)ethyl)isophthalamide;
N1-(1-(furan-2-yl)ethyl)-N3-((2S,3R)-3-hydroxy-4-((R)-1-(3-methoxyphenyl)ethylamino)-1-phenylbutan-2-yl)isophthalamide;
N1-((2S,3R)-3-hydroxy-4-((R)-1-(3-methoxyphenyl)ethylamino)-1-phenylbutan-2-yl)-N3-(1-o-tolylethyl)isophthalamide;
N1-((2S,3R)-3-hydroxy-1-phenyl-4-((S)-1-phenylethylamino)butan-2-yl)-N3-((R)-1-(3-methoxyphenyl)ethyl)-5-methylisophthalamide;
N1-((2S,3R)-3-hydroxy-1-phenyl-4-((S)-1-phenylethylamino)butan-2-yl)-5-methyl-N3-(1-(pyrazin-2-yl)ethyl)isophthalamide;
N1-((2S,3R)-3-hydroxy-4-(methyl((S)-1-phenylethyl)amino)-1-phenylbutan-2-yl)-5-methyl-N3-(1-(pyrazin-2-yl)ethyl)isophthalamide;
N1-((2S,3R)-3-hydroxy-1-phenyl-4-((R)-1-p-tolylethylamino)butan-2-yl)-N3,5-dimethyl-N3-((S)-1-phenylethyl)isophthalamide;
N1-((2S,3R)-3-hydroxy-1-phenyl-4-((R)-1-p-tolylethylamino)butan-2-yl)-N3-((R)-1-(4-methoxyphenyl)ethyl)-5-methylisophthalamide;
N1-((2S,3R)-3-hydroxy-1-phenyl-4-((R)-1-p-tolylethylamino)butan-2-yl)-N3-((R)-1-(3-methoxyphenyl)ethyl)-5-methylisophthalamide;
N1-(1-(furan-2-yl)ethyl)-N3-((2S,3R)-3-hydroxy-1-phenyl-4-((R)-1-p-tolylethylamino)butan-2-yl)-5-methylisophthalamide;
N1-((2S,3R)-3-hydroxy-1-phenyl-4-((R)-1-p-tolylethylamino)butan-2-yl)-5-methyl-N3-(1-(5-methylfuran-2-yl)ethyl)isophthalamide;
N1-((2S,3R)-3-hydroxy-1-phenyl-4-((R)-1-p-tolylethylamino)butan-2-yl)-5-methyl-N3-(1-(pyrazin-2-yl)ethyl)isophthalamide;
N1-((2S,3R)-3-hydroxy-4-((R)-1-(4-methoxyphenyl)ethylamino)-1-phenylbutan-2-yl)-5-methyl-N3-((S)-1-phenylethyl)isophthalamide;
N1-((2S,3R)-3-hydroxy-4-((R)-1-(4-methoxyphenyl)ethylamino)-1-phenylbutan-2-yl)-5-methyl-N3-((R)-1-p-tolylethyl)isophthalamide;
N1-((2S,3R)-3-hydroxy-4-((R)-1-(4-methoxyphenyl)ethylamino)-1-phenylbutan-2-yl)-N3-((R)-1-(4-methoxyphenyl)ethyl)-5-methylisophthalamide;
N1-((2S,3R)-3-hydroxy-4-((R)-1-(4-methoxyphenyl)ethylamino)-1-phenylbutan-2-yl)-5-methyl-N3-(1-(5-methylfuran-2-yl)ethyl)isophthalamide;
N1-((2S,3R)-3-hydroxy-4-((R)-1-(4-methoxyphenyl)ethylamino)-1-phenylbutan-2-yl)-5-methyl-N3-(1-(pyrazin-2-yl)ethyl)isophthalamide;
N1-((2S,3R)-3-hydroxy-4-((R)-1-(4-methoxyphenyl)ethylamino)-1-phenylbutan-2-yl)-5-methyl-N3-(1-(5-methylthiophen-2-yl)ethyl)isophthalamide;
N1-((2S,3R)-3-hydroxy-4-((R)-1-(4-methoxyphenyl)ethylamino)-1-phenylbutan-2-yl)-5-methyl-N3-(1-(3-methylthiophen-2-yl)ethyl)isophthalamide;
N1-((2S,3R)-3-hydroxy-4-((R)-1-(3-methoxyphenyl)ethylamino)-1-phenylbutan-2-yl)-5-methyl-N3-((S)-1-phenylethyl)isophthalamide;
N1-((2S,3R)-3-hydroxy-4-((R)-1-(3-methoxyphenyl)ethylamino)-1-phenylbutan-2-yl)-5-methyl-N3-((R)-1-p-tolylethyl)isophthalamide;
N1-((2S,3R)-3-hydroxy-4-((R)-1-(3-methoxyphenyl)ethylamino)-1-phenylbutan-2-yl)-N3-((R)-1-(4-methoxyphenyl)ethyl)-5-methylisophthalamide;
N1-((2S,3R)-3-hydroxy-4-((R)-1-(3-methoxyphenyl)ethylamino)-1-phenylbutan-2-yl)-N3-((R)-1-(3-methoxyphenyl)ethyl)-5-methylisophthalamide;
N1-(1-(furan-2-yl)ethyl)-N3-((2S,3R)-3-hydroxy-4-((R)-1-(3-methoxyphenyl)ethylamino)-1-phenylbutan-2-yl)-5-methylisophthalamide;
N1-((2S,3R)-3-hydroxy-4-((R)-1-(3-methoxyphenyl)ethylamino)-1-phenylbutan-2-yl)-5-methyl-N3-(1-(5-methylfuran-2-yl)ethyl)isophthalamide;
N1-((2S,3R)-3-hydroxy-4-((R)-1-(3-methoxyphenyl)ethylamino)-1-phenylbutan-2-yl)-5-methyl-N3-(1-o-tolylethyl)isophthalamide;
N1-((2S,3R)-3-hydroxy-4-((R)-1-(3-methoxyphenyl)ethylamino)-1-phenylbutan-2-yl)-5-methyl-N3-(1-(5-methylthiophen-2-yl)ethyl)isophthalamide;
N1-((2S,3R)-3-hydroxy-4-((R)-1-(3-methoxyphenyl)ethylamino)-1-phenylbutan-2-yl)-5-methyl-N3-(1-(3-methylthiophen-2-yl)ethyl)isophthalamide;
N1-((2S,3R)-3-hydroxy-4-((R)-1-(3-methoxyphenyl)ethylamino)-1-phenylbutan-2-yl)-N3-(1-(3-methoxyphenyl)ethyl)-N3,5-dimethylisophthalamide;
N1-((2S,3R)-4-(1-(furan-2-yl)ethylamino)-3-hydroxy-1-phenylbutan-2-yl)-5-methyl-N3-((R)-1-p-tolylethyl)isophthalamide;
N1-(1-(furan-2-yl)ethyl)-N3-((2S,3R)-3-hydroxy-4-(methyl((S)-1-phenylethyl)amino)-1-phenylbutan-2-yl)-5-(N-methylmethylsulfonamido)isophthalamide;
N1-((2S,3R)-3-hydroxy-1-phenyl-4-((R)-1-p-tolylethylamino)butan-2-yl)-5-(N-methylmethylsulfonamido)-N3-((R)-1-p-tolylethyl)isophthalamide;
N1-(1-(furan-2-yl)ethyl)-N3-((2S,3R)-3-hydroxy-1-phenyl-4-((R)-1-p-tolylethylamino)butan-2-yl)-5-(N-methylmethylsulfonamido)isophthalamide;
N1-((2S,3R)-3-hydroxy-1-phenyl-4-((R)-1-p-tolylethylamino)butan-2-yl)-5-(N-methylmethylsulfonamido)-N3-(1-o-tolylethyl)isophthalamide;
N1-((2S,3R)-3-hydroxy-4-((R)-1-(3-methoxyphenyl)ethylamino)-1-phenylbutan-2-yl)-N3-((R)-1-(4-methoxyphenyl)ethyl)-5-(N-methylmethylsulfonamido)isophthalamide;
N1-((2S,3R)-3-hydroxy-4-((R)-1-(3-methoxyphenyl)ethylamino)-1-phenylbutan-2-yl)-5-(N-methylmethylsulfonamido)-N3-(1-o-tolylethyl)isophthalamide;
N1-((2S,3R)-3-hydroxy-1-phenyl-4-((R)-1-p-tolylethylamino)butan-2-yl)-N3-(1-(pyrazin-2-yl)ethyl)isophthalamide;
N1-((2S,3R)-4-(1-(furan-2-yl)ethylamino)-3-hydroxy-1-phenylbutan-2-yl)-N3-((S)-1-phenylethyl)isophthalamide;
N1-((2S,3R)-4-(1-(furan-2-yl)ethylamino)-3-hydroxy-1-phenylbutan-2-yl)-N3-((R)-1-p-tolylethyl)isophthalamide;
N1-((2S,3R)-4-(1-(furan-2-yl)ethylamino)-3-hydroxy-1-phenylbutan-2-yl)-N3-((R)-1-(4-methoxyphenyl)ethyl)isophthalamide;
N1-((2S,3R)-4-(1-(furan-2-yl)ethylamino)-3-hydroxy-1-phenylbutan-2-yl)-N3-((R)-1-(3-methoxyphenyl)ethyl)isophthalamide;
N1-((2S,3R)-4-(1-(furan-2-yl)ethylamino)-3-hydroxy-1-phenylbutan-2-yl)-N3-(1-(3-methylthiophen-2-yl)ethyl)isophthalamide;
N1-(1-(furan-2-yl)ethyl)-N3-((2S,3R)-3-hydroxy-1-phenyl-4-((S)-1-phenylethylamino)butan-2-yl)-5-methylisophthalamide;
N1-((2S,3R)-3-hydroxy-4-(methyl((S)-1-phenylethyl)amino)-1-phenylbutan-2-yl)-5-methyl-N3-(1-(3-methylthiophen-2-yl)ethyl)isophthalamide;
N1-((2S,3R)-3-hydroxy-1-phenyl-4-((R)-1-p-tolylethylamino)butan-2-yl)-5-methyl-N3-((S)-1-phenylethyl)isophthalamide;
N1-((2S,3R)-3-hydroxy-4-((R)-1-(4-methoxyphenyl)ethylamino)-1-phenylbutan-2-yl)-N3-((R)-1-(3-methoxyphenyl)ethyl)-5-methylisophthalamide;
N1-((2S,3R)-3-hydroxy-4-((R)-1-(4-methoxyphenyl)ethylamino)-1-phenylbutan-2-yl)-5-methyl-N3-(1-o-tolylethyl)isophthalamide;
N1-((2S,3R)-4-(1-(furan-2-yl)ethylamino)-3-hydroxy-1-phenylbutan-2-yl)-5-methyl-N3-((S)-1-phenylethyl)isophthalamide;
N1-((2S,3R)-4-(1-(furan-2-yl)ethylamino)-3-hydroxy-1-phenylbutan-2-yl)-N3-((R)-1-(3-methoxyphenyl)ethyl)-5-methylisophthalamide;
N1-((2S,3R)-4-(1-(furan-2-yl)ethylamino)-3-hydroxy-1-phenylbutan-2-yl)-5-methyl-N3-(1-(3-methylthiophen-2-yl)ethyl)isophthalamide;
N1-((2S,3R)-3-hydroxy-1-phenyl-4-((R)-1-p-tolylethylamino)butan-2-yl)-5-(N-methylmethylsulfonamido)-N3-(1-(3-methylthiophen-2-yl)ethyl)isophthalamide;
N1-((2S,3R)-3-hydroxy-4-((R)-1-(4-methoxyphenyl)ethylamino)-1-phenylbutan-2-yl)-N3-methyl-5-(N-methylmethylsulfonamido)-N3-((S)-1-phenylethyl)isophthalamide;
N1-((2S,3R)-3-hydroxy-4-(methyl((S)-1-phenylethyl)amino)-1-phenylbutan-2-yl)-N3-(1-o-tolylethyl)isophthalamide;
N1-((2S,3R)-3-hydroxy-4-((R)-1-(3-methoxyphenyl)ethylamino)-1-phenylbutan-2-yl)-N3-(1-(5-methylthiophen-2-yl)ethyl)isophthalamide;
N1-((2S,3R)-3-hydroxy-4-((R)-1-(3-methoxyphenyl)ethylamino)-1-phenylbutan-2-yl)-N3-(1-(4-methoxyphenyl)ethyl)-N3-methylisophthalamide;
N1-((2S,3R)-3-hydroxy-1-phenyl-4-((S)-1-phenylethylamino)butan-2-yl)-5-methyl-N3-((S)-1-p-tolylethyl)isophthalamide;
N1-(1-(furan-2-yl)ethyl)-N3-((2S,3R)-3-hydroxy-4-((R)-1-(4-methoxyphenyl)ethylamino)-1-phenylbutan-2-yl)-5-methylisophthalamide;
N1-((2S,3R)-4-(1-(furan-2-yl)ethylamino)-3-hydroxy-1-phenylbutan-2-yl)-5-methyl-N3-(1-(5-methylthiophen-2-yl)ethyl)isophthalamide;
N1-((2S,3R)-3-hydroxy-4-(methyl((S)-1-phenylethyl)amino)-1-phenylbutan-2-yl)-5-methyl-N3-(1-(5-methylfuran-2-yl)ethyl)isophthalamide;
N1-((2S,3R)-3-hydroxy-1-phenyl-4-((R)-1-p-tolylethylamino)butan-2-yl)-5-(N-methylmethylsulfonamido)-N3-(1-(5-methylthiophen-2-yl)ethyl)isophthalamide;
N1-((2S,3R)-3-hydroxy-4-((R)-1-(4-methoxyphenyl)ethylamino)-1-phenylbutan-2-yl)-5-(N-methylmethylsulfonamido)-N3-((S)-1-phenylethyl)isophthalamide;
N1-(1-(furan-2-yl)ethyl)-N3-((2S,3R)-3-hydroxy-4-((R)-1-(4-methoxyphenyl)ethylamino)-1-phenylbutan-2-yl)-5-(N-methylmethylsulfonamido)isophthalamide;
N1-(1-(furan-2-yl)ethyl)-N3-((2S,3R)-3-hydroxy-1-phenyl-4-((R)-1-p-tolylethylamino)butan-2-yl)isophthalamide;
N1-((2S,3R)-3-hydroxy-4-((R)-1-(3-methoxyphenyl)ethylamino)-1-phenylbutan-2-yl)-N3-((R)-1-(4-methoxyphenyl)ethyl)isophthalamide;
N1-((2S,3R)-3-hydroxy-1-phenyl-4-((R)-1-p-tolylethylamino)butan-2-yl)-N3-(1-(4-methoxyphenyl)ethyl)-N3,5-dimethylisophthalamide;
N1-((2S,3R)-4-(1-(furan-2-yl)ethylamino)-3-hydroxy-1-phenylbutan-2-yl)-5-methyl-N3-(1-o-tolylethyl)isophthalamide;
N1-(1-(furan-2-yl)ethyl)-N3-((2S,3R)-4-(1-(furan-2-yl)ethylamino)-3-hydroxy-1-phenylbutan-2-yl)-5-methylisophthalamide;
N1-((2S,3R)-3-hydroxy-1-phenyl-4-((R)-1-p-tolylethylamino)butan-2-yl)-5-methyl-N3-(1-(3-methylthiophen-2-yl)ethyl)isophthalamide;
N1-((2S,3R)-3-hydroxy-1-phenyl-4-((S)-1-phenylethylamino)butan-2-yl)-N3-((R)-1-(4-methoxyphenyl)ethyl)-5-(N-methylmethylsulfonamido)isophthalamide;
N1-((2S,3R)-3-hydroxy-4-((R)-1-(3-methoxyphenyl)ethylamino)-1-phenylbutan-2-yl)-5-(N-methylmethylsulfonamido)-N3-(1-(3-methylthiophen-2-yl)ethyl)isophthalamide;
N1-((2S,3R)-4-(1-(furan-2-yl)ethylamino)-3-hydroxy-1-phenylbutan-2-yl)-5-(N-methylmethylsulfonamido)-N3-(1-(3-methylthiophen-2-yl)ethyl)isophthalamide;
N1-((2S,3R)-3-hydroxy-1-phenyl-4-((R)-1-p-tolylethylamino)butan-2-yl)-N3-(1-(4-methoxyphenyl)ethyl)-N3-methylisophthalamide;
N1-((2S,3R)-3-hydroxy-1-phenyl-4-((R)-1-p-tolylethylamino)butan-2-yl)-N3-(1-(5-methylthiophen-2-yl)ethyl)isophthalamide;
N1-((2S,3R)-3-hydroxy-1-phenyl-4-((R)-1-p-tolylethylamino)butan-2-yl)-N3-(1-(3-methoxyphenyl)ethyl)-N3-methylisophthalamide;
N1-((2S,3R)-4-(1-(furan-2-yl)ethylamino)-3-hydroxy-1-phenylbutan-2-yl)-N3-(1-(5-methylfuran-2-yl)ethyl)isophthalamide;
N1-((2S,3R)-3-hydroxy-4-(methyl((S)-1-phenylethyl)amino)-1-phenylbutan-2-yl)-5-methyl-N3-(1-o-tolylethyl)isophthalamide;
N1-((2S,3R)-3-hydroxy-1-phenyl-4-((R)-1-p-tolylethylamino)butan-2-yl)-N3-methyl-5-(N-methylmethylsulfonamido)-N3-((S)-1-phenylethyl)isophthalamide;
N1-((2S,3R)-3-hydroxy-4-((R)-1-(4-methoxyphenyl)ethylamino)-1-phenylbutan-2-yl)-N3-((R)-1-(3-methoxyphenyl)ethyl)-5-(N-methylmethylsulfonamido)isophthalamide;
N1-((2S,3R)-3-hydroxy-4-((R)-1-(3-methoxyphenyl)ethylamino)-1-phenylbutan-2-yl)-5-(N-methylmethylsulfonamido)-N3-(1-(5-methylthiophen-2-yl)ethyl)isophthalamide;
N1-((2S,3R)-4-(1-(furan-2-yl)ethylamino)-3-hydroxy-1-phenylbutan-2-yl)-N3-((R)-1-(4-methoxyphenyl)ethyl)-5-(N-methylmethylsulfonamido)isophthalamide;
N1-((2S,3R)-3-hydroxy-1-phenyl-4-((S)-1-phenylethylamino)butan-2-yl)-N3-(1-(5-methylthiophen-2-yl)ethyl)isophthalamide;
N1-((2S,3R)-3-hydroxy-4-(methyl((S)-1-phenylethyl)amino)-1-phenylbutan-2-yl)-N3-(1-(5-methylfuran-2-yl)ethyl)isophthalamide;
N1-((2S,3R)-3-hydroxy-1-phenyl-4-((R)-1-p-tolylethylamino)butan-2-yl)-N3-((R)-1-(3-methoxyphenyl)ethyl)isophthalamide;
N1-((2S,3R)-3-hydroxy-4-((R)-1-(4-methoxyphenyl)ethylamino)-1-phenylbutan-2-yl)-N3-(1-(5-methylthiophen-2-yl)ethyl)isophthalamide;
N1-((2S,3R)-3-hydroxy-4-((R)-1-(3-methoxyphenyl)ethylamino)-1-phenylbutan-2-yl)-N3-methyl-N3-((S)-1-phenylethyl)isophthalamide;
N1-((2S,3R)-3-hydroxy-4-((R)-1-(3-methoxyphenyl)ethylamino)-1-phenylbutan-2-yl)-N3-(1-(3-methylthiophen-2-yl)ethyl)isophthalamide;
N1-((2S,3R)-3-hydroxy-4-(methyl((S)-1-phenylethyl)amino)-1-phenylbutan-2-yl)-N3,5-dimethyl-N3-((S)-1-phenylethyl)isophthalamide;
N1-((2S,3R)-3-hydroxy-4-(methyl((S)-1-phenylethyl)amino)-1-phenylbutan-2-yl)-N3-(1-(4-methoxyphenyl)ethyl)-N3,5-dimethylisophthalamide;
N1-((2S,3R)-3-hydroxy-1-phenyl-4-((R)-1-p-tolylethylamino)butan-2-yl)-5-methyl-N3-(1-(5-methylthiophen-2-yl)ethyl)isophthalamide;
N1-((2S,3R)-3-hydroxy-4-((R)-1-(4-methoxyphenyl)ethylamino)-1-phenylbutan-2-yl)-N3,5-dimethyl-N3-((S)-1-phenylethyl)isophthalamide;
N1-((2S,3R)-3-hydroxy-4-((R)-1-(4-methoxyphenyl)ethylamino)-1-phenylbutan-2-yl)-N3-(1-(3-methoxyphenyl)ethyl)-N3,5-dimethylisophthalamide;
N1-((2S,3R)-3-hydroxy-4-((R)-1-(3-methoxyphenyl)ethylamino)-1-phenylbutan-2-yl)-N3,5-dimethyl-N3-((S)-1-phenylethyl)isophthalamide;
N1-((2S,3R)-4-(1-(furan-2-yl)ethylamino)-3-hydroxy-1-phenylbutan-2-yl)-5-methyl-N3-(1-(pyridin-2-yl)ethyl)isophthalamide;
N1-((2S,3R)-3-hydroxy-4-((R)-1-(4-methoxyphenyl)ethylamino)-1-phenylbutan-2-yl)-5-methyl-N3-(1-(pyridin-2-yl)ethyl)isophthalamide;
N1-((2S,3R)-4-(1-(furan-2-yl)ethylamino)-3-hydroxy-1-phenylbutan-2-yl)-5-(N-methylmethylsulfonamido)-N3-((S)-1-phenylethyl)isophthalamide;
N1-((2S,3R)-3-hydroxy-1-phenyl-4-((S)-1-phenylethylamino)butan-2-yl)-5-(N-methylmethylsulfonamido)-N3-((R)-1-p-tolylethyl)isophthalamide;
N1-(1-(furan-2-yl)ethyl)-N3-((2S,3R)-3-hydroxy-1-phenyl-4-((S)-1-phenylethylamino)butan-2-yl)-5-(N-methylmethylsulfonamido)isophthalamide;
N1-((2S,3R)-3-hydroxy-1-phenyl-4-((S)-1-phenylethylamino)butan-2-yl)-5-(N-methylmethylsulfonamido)-N3-(1-(3-methylthiophen-2-yl)ethyl)isophthalamide;
N1-((2S,3R)-3-hydroxy-1-phenyl-4-((S)-1-phenylethylamino)butan-2-yl)-N3-(1-(5-methylfuran-2-yl)ethyl)isophthalamide;
N1-((2S,3R)-3-hydroxy-4-(methyl((S)-1-phenylethyl)amino)-1-phenylbutan-2-yl)-N3-(1-(pyrazin-2-yl)ethyl)isophthalamide;
N1-((2S,3R)-3-hydroxy-4-((R)-1-(4-methoxyphenyl)ethylamino)-1-phenylbutan-2-yl)-N3-methyl-N3-((S)-1-phenylethyl)isophthalamide;
N1-((2S,3R)-3-hydroxy-4-(methyl((S)-1-phenylethyl)amino)-1-phenylbutan-2-yl)-5-(N-methylmethylsulfonamido)-N3-((R)-1-p-tolylethyl)isophthalamide;
N1-((2S,3R)-3-hydroxy-4-(methyl((S)-1-phenylethyl)amino)-1-phenylbutan-2-yl)-N3-(1-(4-methoxyphenyl)ethyl)-N3-methyl-5-(N-methylmethylsulfonamido)isophthalamide;
N1-((2S,3R)-3-hydroxy-4-(methyl((S)-1-phenylethyl)amino)-1-phenylbutan-2-yl)-N3-(1-(5-methylthiophen-2-yl)ethyl)isophthalamide;
N1-((2S,3R)-3-hydroxy-1-phenyl-4-((R)-1-p-tolylethylamino)butan-2-yl)-N3-((S)-1-phenylethyl)isophthalamide;
N1-((2S,3R)-3-hydroxy-4-(methyl((S)-1-phenylethyl)amino)-1-phenylbutan-2-yl)-N3-((R)-1-(3-methoxyphenyl)ethyl)isophthalamide;
N1-((2S,3R)-3-hydroxy-1-phenyl-4-((R)-1-p-tolylethylamino)butan-2-yl)-5-methyl-N3-(1-(pyridin-2-yl)ethyl)isophthalamide;
N1-((2S,3R)-3-hydroxy-1-phenyl-4-((S)-1-phenylethylamino)butan-2-yl)-5-(N-methylmethylsulfonamido)-N3-((S)-1-phenylethyl)isophthalamide;
N1-((2S,3R)-3-hydroxy-4-((R)-1-(3-methoxyphenyl)ethylamino)-1-phenylbutan-2-yl)-N3-((R)-1-p-tolylethyl)isophthalamide;
N1-((2S,3R)-4-(1-(furan-2-yl)ethylamino)-3-hydroxy-1-phenylbutan-2-yl)-5-(N-methylmethylsulfonamido)-N3-(1-(5-methylthiophen-2-yl)ethyl)isophthalamide;
N1-((2S,3R)-3-hydroxy-1-phenyl-4-((S)-1-phenylethylamino)butan-2-yl)-5-methyl-N3-(1-(pyridin-2-yl)ethyl)isophthalamide;
N1-((2S,3R)-3-hydroxy-4-((R)-1-(4-methoxyphenyl)ethylamino)-1-phenylbutan-2-yl)-N3-(1-(3-methoxyphenyl)ethyl)-N3-methylisophthalamide;
N1-((2S,3R)-3-hydroxy-4-((R)-1-(4-methoxyphenyl)ethylamino)-1-phenylbutan-2-yl)-N3-(1-(5-methylfuran-2-yl)ethyl)-5-(N-methylmethylsulfonamido)isophthalamide;
N1-((2S,3R)-3-hydroxy-1-phenyl-4-((S)-1-phenylethylamino)butan-2-yl)-N3-(1-(3-methoxyphenyl)ethyl)-N3,5-dimethylisophthalamide; or
N1-((2S,3R)-3-hydroxy-4-(methyl((S)-1-phenylethyl)amino)-1-phenylbutan-2-yl)-5-methyl-N3-((S)-1-phenylethyl)isophthalamide;
N1-((2S,3R)-4-(3-(diethylamino)benzylamino)-3-hydroxy-1-phenylbutan-2-yl)-N3-methyl-N3-((4-methylthiazol-2-yl)methyl)isophthalamide;
5-(dimethylamino)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
N1-((2S,3R)-4-(3-(benzylamino)benzylamino)-3-hydroxy-1-phenylbutan-2-yl)-N3-methyl-N3-((4-methylthiazol-2-yl)methyl)isophthalamide;
N1-((2S,3R)-3-hydroxy-4-(3-methoxyphenylamino)-1-phenylbutan-2-yl)-N3-methyl-N3-((4-methylthiazol-2-yl)methyl)isophthalamide;
N1-((2S,3R)-3-hydroxy-4-(3-(methylamino)benzylamino)-1-phenylbutan-2-yl)-N3-methyl-N3-((4-methylthiazol-2-yl)methyl)isophthalamide;
5-(diethylamino)-N1-((2S,3R)-3-hydroxy-4-(3-methoxybenzylamino)-1-phenylbutan-2-yl)-N3-methyl-N3-((4-methylthiazol-2-yl)methyl)isophthalamide;
5-(diethylamino)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
3-(((2R,3S)-3-(3-(dimethylamino)-5-(methyl((4-methylthiazol-2-yl)methyl)carbamoyl)benzamido)-2-hydroxy-4-phenylbutylamino)methyl)-5-isopropylphenyl methanesulfonate;
N1-((2S,3R)-3-hydroxy-4-(3-methoxybenzylamino)-1-phenylbutan-2-yl)-N3-methyl-N3-((4-methylthiazol-2-yl)methyl)-5-(pyrrolidin-1-yl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyrrolidin-1-yl)isophthalamide;
N1-((2S,3R)-4-(3,5-bis(dimethylamino)benzylamino)-3-hydroxy-1-phenylbutan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-N3-((4-methylthiazol-2-yl)methyl)-5-(pyrrolidin-1-yl)isophthalamide;
N1-((2S,3R)-4-(3-aminobenzylamino)-3-hydroxy-1-phenylbutan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-N3-methyl-N3-((4-methylthiazol-2-yl)methyl)-5-(2-oxopyrrolidin-1-yl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(2-oxopyrrolidin-1-yl)isophthalamide;
N1-((2S,3R)-3-hydroxy-4-(3-methoxybenzylamino)-1-phenylbutan-2-yl)-N3-((4-methylthiazol-2-yl)methyl)-5-(2-oxopyrrolidin-1-yl)isophthalamide;
N1-((2S,3R)-4-((2-amino-6-methoxypyrimidin-4-yl)methylamino)-3-hydroxy-1-phenylbutan-2-yl)-N3-methyl-N3-((4-methylthiazol-2-yl)methyl)isophthalamide;
N1-((2S,3R)-4-((2-(dimethylamino)-6-methoxypyrimidin-4-yl)methylamino)-3-hydroxy-1-phenylbutan-2-yl)-N3-methyl-N3-((4-methylthiazol-2-yl)methyl)isophthalamide;
N1-((2S,3R)-3-hydroxy-4-(3-methoxy-5-nitrobenzylamino)-1-phenylbutan-2-yl)-N3-methyl-N3-((4-methylthiazol-2-yl)methyl)isophthalamide;
N1-((2S,3R)-4-(3-(dimethylamino)-5-methoxybenzylamino)-3-hydroxy-1-phenylbutan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-N3-methyl-N3-((4-methylthiazol-2-yl)methyl)-5-(piperidin-1-yl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(piperidin-1-yl)isophthalamide;
N1-((2S,3R)-3-hydroxy-1-phenyl-4-(3-(trifluoromethyl)benzylamino)butan-2-yl)-N3-methyl-N3-((4-methylthiazol-2-yl)methyl)-5-(2-oxopyrrolidin-1-yl)isophthalamide;
N1-((2S,3R)-3-hydroxy-4-(3-methoxybenzylamino)-1-phenylbutan-2-yl)-N3-methyl-N3-((4-methylthiazol-2-yl)methyl)-5-(1H-pyrrol-1-yl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrrol-1-yl)isophthalamide;
N1-((2S,3R)-3-hydroxy-4-(1-(5-isopropylpyridin-3-yl)ethylamino)-1-phenylbutan-2-yl)-N3-methyl-N3-((4-methylthiazol-2-yl)methyl)isophthalamide;
N1-((2S,3R)-3-hydroxy-4-((S)-1-(5-isopropylpyridin-3-yl)ethylamino)-1-phenylbutan-2-yl)-N3-methyl-N3-((4-methylthiazol-2-yl)methyl)isophthalamide;
N1-((2S,3R)-3-hydroxy-4-((R)-1-(5-isopropylpyridin-3-yl)ethylamino)-1-phenylbutan-2-yl)-N3-methyl-N3-((4-methylthiazol-2-yl)methyl)isophthalamide;
N1-((2S,3R)-3-hydroxy-4-(3-isopropyl-5-(N-methylmethylsulfonamido)benzylamino)-1-phenylbutan-2-yl)-N3-methyl-N3-((4-methylthiazol-2-yl)methyl)isophthalamide;
N1-((2S,3R)-3-hydroxy-4-(3-isopropyl-5-(methylsulfonyl)benzylamino)-1-phenylbutan-2-yl)-N3-methyl-N3-((4-methylthiazol-2-yl)methyl)isophthalamide;
N1-((2S,3R)-3-hydroxy-4-((5-isopropyl-2-methylpyridin-3-yl)methylamino)-1-phenylbutan-2-yl)-N3-methyl-N3-((4-methylthiazol-2-yl)methyl)isophthalamide;
N1-((2S,3R)-3-hydroxy-4-(3-hydroxy-5-(prop-1-en-2-yl)benzylamino)-1-phenylbutan-2-yl)-N3-methyl-N3-((4-methylthiazol-2-yl)methyl)isophthalamide;
3-(((2R,3S)-2-hydroxy-3-(3-(methyl((4-methylthiazol-2-yl)methyl)carbamoyl)benzamido)-4-phenylbutylamino)methyl)-5-isopropylphenyl methanesulfonate;
N1-((2S,3R)-3-hydroxy-4-(3-isopropyl-5-(methylsulfonamidomethyl)benzylamino)-1-phenylbutan-2-yl)-N3-methyl-N3-((4-methylthiazol-2-yl)methyl)isophthalamide;
N1-((2S,3R)-4-(3-(ethylsulfonyl)-5-isopropylbenzylamino)-3-hydroxy-1-phenylbutan-2-yl)-N3-methyl-N3-((4-methylthiazol-2-yl)methyl)isophthalamide;
N1-((2S,3R)-4-(3-acetamido-5-isopropylbenzylamino)-3-hydroxy-1-phenylbutan-2-yl)-N3-methyl-N3-((4-methylthiazol-2-yl)methyl)isophthalamide;
3-(((2R,3S)-2-hydroxy-3-(3-(methyl((4-methylthiazol-2-yl)methyl)carbamoyl)benzamido)-4-phenylbutylamino)methyl)-5-isopropylphenyl dimethylcarbamate;
methyl 3-(((2R,3S)-2-hydroxy-3-(3-(methyl((4-methylthiazol-2-yl)methyl)carbamoyl)benzamido)-4-phenylbutylamino)methyl)-5-isopropylphenylcarbamate;
3-(((2R,3S)-3-(3-(dimethylamino)-5-(methyl((4-methylthiazol-2-yl)methyl)carbamoyl)benzamido)-2-hydroxy-4-phenylbutylamino)methyl)-5-isopropylphenyl dimethylcarbamate;
N1-((2S,3R)-4-(3-tert-butyl-5-hydroxybenzylamino)-3-hydroxy-1-phenylbutan-2-yl)-N3-methyl-N3-((4-methylthiazol-2-yl)methyl)isophthalamide;
N1-((2S,3R)-3-hydroxy-4-(3-isopropyl-5-(methylsulfonylmethyl)benzylamino)-1-phenylbutan-2-yl)-N3-methyl-N3-((4-methylthiazol-2-yl)methyl)isophthalamide;
N1-((2S,3R)-3-hydroxy-4-(3-isopropyl-5-(isopropylsulfonyl)benzylamino)-1-phenylbutan-2-yl)-N3-methyl-N3-((4-methylthiazol-2-yl)methyl)isophthalamide;
3-(((2R,3S)-2-hydroxy-3-(3-(methyl((4-methylthiazol-2-yl)methyl)carbamoyl)benzamido)-4-phenylbutylamino)methyl)-5-isopropylphenyl morpholine-4-carboxylate;
N1-((2S,3R)-4-((1-tert-butyl-1H-pyrazol-4-yl)methylamino)-3-hydroxy-1-phenylbutan-2-yl)-5-(N-methylmethylsulfonamido)-N3-((R)-1-phenylethyl)isophthalamide;
N1-((2S,3S)-3-hydroxy-4-((5-isopropylpyridin-3-yl)methylamino)-1-phenylbutan-2-yl)-N3-methyl-N3-((4-methylthiazol-2-yl)methyl)isophthalamide;
N1-((2R,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
N1-((2S,3R)-4-((2-amino-6-isopropylpyrimidin-4-yl)methylamino)-3-hydroxy-1-phenylbutan-2-yl)-N3-methyl-N3-((4-methylthiazol-2-yl)methyl)isophthalamide;
N1-((2S,3R)-3-hydroxy-4-((6-isopropyl-2-(methylamino)pyrimidin-4-yl)methylamino)-1-phenylbutan-2-yl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-2-yl)isophthalamide;
N1-((2S,3R)-4-((2-amino-6-isopropylpyrimidin-4-yl)methylamino)-3-hydroxy-1-phenylbutan-2-yl)-N3-methyl-N3-((4-methylthiazol-2-yl)methyl)-5-(oxazol-2-yl)isophthalamide;
N1-cyclopropyl-N3-((2S,3R)-3-hydroxy-4-((5-isopropylpyridin-3-yl)methylamino)-1-phenylbutan-2-yl)-N1-((4-methylthiazol-2-yl)methyl)-5-(oxazol-2-yl)isophthalamide;
N1-((2S,3R)-3-hydroxy-4-((5-isopropylpyridin-3-yl)methylamino)-1-phenylbutan-2-yl)-5-(hydroxymethyl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-N3-((4-methylthiazol-2-yl)methyl)-5-(oxazol-2-yl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(oxazol-5-yl)isophthalamide;
N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-5-yl)isophthalamide;
5-acetyl-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
methyl 3-(((2R,3S)-2-hydroxy-3-(3-(methyl((4-methylthiazol-2-yl)methyl)carbamoyl)benzamido)-4-phenylbutylamino)methyl)benzoate;
N1-((2S,3R)-4-(3-(difluoromethyl)benzylamino)-3-hydroxy-1-phenylbutan-2-yl)-5-(N-methylmethylsulfonamido)-N3-((R)-1-phenylethyl)isophthalamide;
N1-((2S,3R)-4-(3-(difluoromethyl)benzylamino)-3-hydroxy-1-phenylbutan-2-yl)-N3-methyl-N3-((4-methylthiazol-2-yl)methyl)isophthalamide;
N1-((2S,3R)-4-(3-(1,1-difluoroethyl)benzylamino)-3-hydroxy-1-phenylbutan-2-yl)-5-(N-methylmethylsulfonamido)-N3-((R)-1-phenylethyl)isophthalamide;
N1-((2S,3R)-4-(3-(1,1-difluoroethyl)benzylamino)-3-hydroxy-1-phenylbutan-2-yl)-N3-methyl-N3-((4-methylthiazol-2-yl)methyl)isophthalamide;
N1-((2S,3R)-4-(bis((1-methyl-1H-pyrazol-4-yl)methyl)amino)-3-hydroxy-1-phenylbutan-2-yl)-N3-methyl-N3-((4-methylthiazol-2-yl)methyl)isophthalamide;
N1-((2S,3R)-4-(1-(3-(difluoromethyl)phenyl)ethylamino)-3-hydroxy-1-phenylbutan-2-yl)-N3-methyl-N3-((4-methylthiazol-2-yl)methyl)isophthalamide;
N1-((2S,3R)-3-hydroxy-4-(3-(1-hydroxyethyl)benzylamino)-1-phenylbutan-2-yl)-N3-methyl-N3-((4-methylthiazol-2-yl)methyl)isophthalamide;
N1-((2S,3R)-3-hydroxy-1-phenyl-4-(3-(prop-1-en-2-yl)benzylamino)butan-2-yl)-N3-methyl-N3-((4-methylthiazol-2-yl)methyl)isophthalamide;
N1-((2S,3R)-4-(3-(1-chloroethyl)benzylamino)-3-hydroxy-1-phenylbutan-2-yl)-N3-methyl-N3-((4-methylthiazol-2-yl)methyl)isophthalamide;
5-(fluoromethyl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
N1-((2S,3R)-3-hydroxy-4-(3-(2-hydroxypropan-2-yl)benzylamino)-1-phenylbutan-2-yl)-N3-methyl-N3-((4-methylthiazol-2-yl)methyl)isophthalamide;
N1-((2S,3R)-4-((5-tert-butylpyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-1-phenyl-4-((5-phenylisoxazol-3-yl)methylamino)butan-2-yl)-N3-methyl-N3-((4-methylthiazol-2-yl)methyl)isophthalamide;
N1-((2S,3R)-3-hydroxy-4-((R)-1-(3-methoxyphenyl)ethylamino)-1-phenylbutan-2-yl)-5-(N-methylmethylsulfonamido)-N3-((R)-1-phenylethyl)isophthalamide;
5-(difluoromethyl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
N1-((2S,3R)-1-(3,5-difluorophenyl)-3-hydroxy-4-((5-isopropylpyridin-3-yl)methylamino)butan-2-yl)-N3-methyl-N3-((4-methylthiazol-2-yl)methyl)isophthalamide;
N1-((2S,3R)-4-((5-(1,1-difluoroethyl)pyridin-3-yl)methylamino)-3-hydroxy-1-phenylbutan-2-yl)-N3-methyl-N3-((4-methylthiazol-2-yl)methyl)isophthalamide;
5-(dimethylamino)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide;
N1-((2S,3R)-4-((5-tert-butylpyridin-3-yl)methylamino)-3-hydroxy-1-phenylbutan-2-yl)-5-(dimethylamino)-N3-methyl-N3-((4-methylthiazol-2-yl)methyl)isophthalamide;
N1-((2S,3R)-4-(3-(1,1-difluoroethyl)benzylamino)-3-hydroxy-1-phenylbutan-2-yl)-5-(dimethylamino)-N3-methyl-N3-((4-methylthiazol-2-yl)methyl)isophthalamide;
N1-((2S,3R)-3-hydroxy-4-(3-methoxybenzylamino)-1-phenylbutan-2-yl)-N3-methyl-N3-((4-methylpyrimidin-2-yl)methyl)isophthalamide;
N1-((2S,3R)-4-(3-cyanobenzylamino)-3-hydroxy-1-phenylbutan-2-yl)-N3-methyl-N3-((4-methylthiazol-2-yl)methyl)isophthalamide;
2′-cyano-N3-((2S,3R)-3-hydroxy-4-(3-methoxybenzylamino)-1-phenylbutan-2-yl)-N5-methyl-N5-((4-methylthiazol-2-yl)methyl)biphenyl-3,5-dicarboxamide;
N1-((2S,3R)-4-(3-cyanobenzylamino)-3-hydroxy-1-phenylbutan-2-yl)-5-(N-methylmethylsulfonamido)-N3-((R)-1-phenylethyl)isophthalamide;
2′-cyano-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide;
N1-((2S,3R)-4-((5-bromopyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-4-(2-(3-methoxyphenyl)propan-2-ylamino)-1-phenylbutan-2-yl)-N3-methyl-N3-((4-methylthiazol-2-yl)methyl)isophthalamide;
N1-((2S,3R)-4-((5-cyanopyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-N3-methyl-N3-((4-methylthiazol-2-yl)methyl)-5-(pyridin-2-yl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-2-yl)isophthalamide;
N1-((2S,3R)-3-hydroxy-4-((5-isopropylpyridin-3-yl)methylamino)-1-(thiazol-4-yl)butan-2-yl)-N3-methyl-N3-((4-methylthiazol-2-yl)methyl)isophthalamide;
N1-((2S,3R)-3-hydroxy-4-(3-methoxybenzylamino)-1-phenylbutan-2-yl)-N3-methyl-N3-((4-methylthiazol-2-yl)methyl)-5-(pyridin-3-yl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-3-yl)isophthalamide;
N1-((2S,3R)-3-hydroxy-4-(3-methoxybenzylamino)-1-phenylbutan-2-yl)-N3-methyl-N3-((4-methylthiazol-2-yl)methyl)-5-(pyridin-4-yl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyridin-4-yl)isophthalamide;
N3-((2S,3R)-3-hydroxy-4-(3-methoxybenzylamino)-1-phenylbutan-2-yl)-2′-methoxy-N5-methyl-N5-((4-methylthiazol-2-yl)methyl)biphenyl-3,5-dicarboxamide;
N1-((2S,3R)-3-hydroxy-4-((5-isopropylpyridin-3-yl)methylamino)-1-phenylbutan-2-yl)-5-(oxazol-2-yl)-N3-((R)-1-phenylethyl)isophthalamide;
N1-((2S,3R)-3-hydroxy-4-(3-methoxybenzylamino)-1-phenylbutan-2-yl)-5-(oxazol-2-yl)-N3-((R)-1-phenylethyl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(pyrazin-2-yl)isophthalamide;
N1-((2S,3R)-4-((5-chloropyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-1-phenyl-4-(3-(trifluoromethyl)benzylamino)butan-2-yl)-N3-methyl-N3-((4-methylthiazol-2-yl)methyl)-5-(pyrazin-2-yl)isophthalamide;
N1-((2S,3R)-4-((5-fluoropyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-1-phenyl-4-(3-(trifluoromethyl)benzylamino)butan-2-yl)-N3,5-dimethyl-N3-((4-methylthiazol-2-yl)methyl)isophthalamide;
N1-((2S,3R)-4-(3,5-dichlorobenzylamino)-3-hydroxy-1-phenylbutan-2-yl)-N3-methyl-N3-((4-methylthiazol-2-yl)methyl)isophthalamide;
3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoic acid;
N1-((2S,3R)-3-hydroxy-4-((5-isopropylpyridin-3-yl)methylamino)-1-phenylbutan-2-yl)-5-(1-hydroxyethyl)-N3-methyl-N3-((4-methylthiazol-2-yl)methyl)isophthalamide;
methyl 3-(((2R,3S)-2-hydroxy-3-(3-(methyl((4-methylthiazol-2-yl)methyl)carbamoyl)benzamido)-4-phenylbutylamino)methyl)-5-(N-methylmethylsulfonamido)benzoate;
N1-((2S,3R)-3-hydroxy-4-((5-isopropylpyridin-3-yl)methylamino)-1-phenylbutan-2-yl)-N3-methyl-N3-((3-methyl-1,2,4-oxadiazol-5-yl)methyl)isophthalamide;
N1-((2S,3R)-3-hydroxy-4-((5-isopropylpyridin-3-yl)methylamino)-1-phenylbutan-2-yl)-N3-methyl-N3-((5-methyl-1,2,4-oxadiazol-3-yl)methyl)isophthalamide;
5-(difluoromethyl)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide;
N1-((2S,3R)-4-((5-(1,1-difluoroethyl)pyridin-3-yl)methylamino)-3-hydroxy-1-phenylbutan-2-yl)-5-(difluoromethyl)-N3-methyl-N3-((4-methylthiazol-2-yl)methyl)isophthalamide;
5-(difluoromethyl)-N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide;
N1-((2S,3R)-4-((5-(1,1-difluoroethyl)pyridin-3-yl)methylamino)-3-hydroxy-1-phenylbutan-2-yl)-5-(dimethylamino)-N3-methyl-N3-((4-methylthiazol-2-yl)methyl)isophthalamide;
5-(dimethylamino)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide;
5-(dimethylamino)-N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)isophthalamide;
5-(difluoromethyl)-N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)isophthalamide;
N1-((2S,3R)-3-hydroxy-1-phenyl-4-(3-(trifluoromethyl)benzylamino)butan-2-yl)-5-isopropyl-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-5-isopropyl-N3-methyl-N3-((4-methylthiazol-2-yl)methyl)isophthalamide;
N1-((2S,3R)-4-((5-(2-fluoropropan-2-yl)pyridin-3-yl)methylamino)-3-hydroxy-1-phenylbutan-2-yl)-5-isopropyl-N3-methyl-N3-((4-methylthiazol-2-yl)methyl)isophthalamide;
N1-((4-(fluoromethyl)thiazol-2-yl)methyl)-N3-((2S,3R)-3-hydroxy-4-((5-isopropylpyridin-3-yl)methylamino)-1-phenylbutan-2-yl)-N1-methylisophthalamide;
N1-((2S,3R)-3-hydroxy-4-((5-((Z)-1-methoxyprop-1-en-2-yl)pyridin-3-yl)methylamino)-1-phenylbutan-2-yl)-N3-methyl-N3-((4-methylthiazol-2-yl)methyl)isophthalamide;
N1-(4-(fluoromethyl)thiazol-2-yl)methyl)-N3-((2S,3R)-3-hydroxy-1-phenyl-4-((5-(trifluoromethyl)pyridin-3-yl)methylamino)butan-2-yl)-N1-methylisophthalamide;
N1-(4-(fluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide;
N1-((2S,3R)-4-((5-(1,1-dimethoxypropan-2-yl)pyridin-3-yl)methylamino)-3-hydroxy-1-phenylbutan-2-yl)-N3-methyl-N3-((4-methylthiazol-2-yl)methyl)isophthalamide;
N1-(4-(difluoromethyl)thiazol-2-yl)methyl)-N3-((2S,3R)-3-hydroxy-4-((5-isopropylpyridin-3-yl)methylamino)-1-phenylbutan-2-yl)-N1-methylisophthalamide;
N1-(4-(difluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide;
N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-2-yl)isophthalamide;
N1-((2S,3R)-3-hydroxy-4-((R)-1-(3-methoxyphenyl)ethylamino)-1-phenylbutan-2-yl)-N3-methyl-N3-((4-methylthiazol-2-yl)methyl)-5-(oxazol-2-yl)isophthalamide;
N1-((2S,3R)-3-hydroxy-4-((S)-1-(3-methoxyphenyl)ethylamino)-1-phenylbutan-2-yl)-N3-methyl-N3-((4-methylthiazol-2-yl)methyl)-5-(oxazol-2-yl)isophthalamide;
N1-((2S,3R)-3-hydroxy-4-(3-methoxybenzylamino)-1-phenylbutan-2-yl)-5-(3-hydroxypyrrolidin-1-yl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-5-(3-hydroxypyrrolidin-1-yl)-N3-methyl-N3-((4-methylthiazol-2-yl)methyl)isophthalamide;
N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)-5-(1H-pyrrol-1-yl)isophthalamide;
N1-((2S,3R)-3-hydroxy-4-(3-methoxybenzylamino)-1-phenylbutan-2-yl)-5-(1H-imidazol-1-yl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-5-(1H-imidazol-1-yl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-5-(1H-imidazol-2-yl)-N3-methyl-N3-((4-methylthiazol-2-yl)methyl)isophthalamide;
2-hydroxy-N1-((2S,3R)-3-hydroxy-4-(3-methoxybenzylamino)-1-phenylbutan-2-yl)-N3-methyl-N3-((4-methylthiazol-2-yl)methyl)isophthalamide;
2-hydroxy-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
5-(3,3-dimethoxypyrrolidin-1-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
N1-((2S,3R)-3-hydroxy-4-((4-methylthiazol-2-yl)methylamino)-1-phenylbutan-2-yl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-N3-methyl-5-(1-methyl-1H-imidazol-2-yl)-N3-((4-methylthiazol-2-yl)methyl)isophthalamide;
N1-((2S,3R)-4-(3-cyano-5-isopropylbenzylamino)-3-hydroxy-1-phenylbutan-2-yl)-N3-methyl-N3-((4-methylthiazol-2-yl)methyl)isophthalamide;
N1-((2S,3R)-4-(5-chloro-2-fluorobenzylamino)-3-hydroxy-1-phenylbutan-2-yl)-N3-methyl-N3-((4-methylthiazol-2-yl)methyl)isophthalamide;
N1-((2S,3R)-3-hydroxy-1-phenyl-4-((4-(trifluoromethyl)pyridin-2-yl)methylamino)butan-2-yl)-N3-methyl-N3-((4-methylthiazol-2-yl)methyl)isophthalamide;
N1-((2S,3R)-3-hydroxy-4-((5-(isopropylamino)pyridin-3-yl)methylamino)-1-phenylbutan-2-yl)-N3-methyl-N3-((4-methylthiazol-2-yl)methyl)isophthalamide;
4′-(dimethylamino)-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide;
N1-((2S,3R)-4-(3-bromo-5-(trifluoromethyl)benzylamino)-3-hydroxy-1-phenylbutan-2-yl)-N3-methyl-N3-((4-methylthiazol-2-yl)methyl)isophthalamide;
N1-((2S,3R)-4-(3-cyano-5-(trifluoromethyl)benzylamino)-3-hydroxy-1-phenylbutan-2-yl)-N3-methyl-N3-((4-methylthiazol-2-yl)methyl)isophthalamide;
N1-((2S,3R)-4-(biphenyl-3-ylmethylamino)-3-hydroxy-1-phenylbutan-2-yl)-N3-methyl-N3-((4-methylthiazol-2-yl)methyl)isophthalamide;
N1-((2S,3R)-4-(3-tert-butylbenzylamino)-3-hydroxy-1-phenylbutan-2-yl)-5-(dimethylamino)-N3-methyl-N3-((4-methylthiazol-2-yl)methyl)isophthalamide;
3′-chloro-N3-((2S,3R)-3-hydroxy-4-((5-isopropylpyridin-3-yl)methylamino)-1-phenylbutan-2-yl)-N5-methyl-N5-((4-methylthiazol-2-yl)methyl)biphenyl-3,5-dicarboxamide;
N1-((2S,3R)-3-hydroxy-1-phenyl-4-(3-(pyridin-4-yl)-5-(trifluoromethyl)benzylamino)butan-2-yl)-N3-methyl-N3-((4-methylthiazol-2-yl)methyl)isophthalamide;
N1-((2S,3R)-4-(3-acetamido-5-(trifluoromethyl)benzylamino)-3-hydroxy-1-phenylbutan-2-yl)-N3-methyl-N3-((4-methylthiazol-2-yl)methyl)isophthalamide;
N1-((2S,3R)-3-hydroxy-4-(2-methoxy-5-(trifluoromethyl)benzylamino)-1-phenylbutan-2-yl)-N3-methyl-N3-((4-methylthiazol-2-yl)methyl)isophthalamide;
N1-((2S,3R)-3-hydroxy-4-(3-(methylamino)-5-(trifluoromethyl)benzylamino)-1-phenylbutan-2-yl)-N3-methyl-N3-((4-methylthiazol-2-yl)methyl)isophthalamide;
N1-((2S,3R)-3-hydroxy-4-((1-methyl-1H-pyrazol-4-yl)methylamino)-1-phenylbutan-2-yl)-N3-methyl-N3-((4-methylthiazol-2-yl)methyl)isophthalamide;
N1-((2S,3R)-3-hydroxy-1-phenyl-4-(5-(prop-1-en-2-yl)nicotinamido)butan-2-yl)-N3-methyl-N3-((4-methylthiazol-2-yl)methyl)isophthalamide;
N1-((2S,3R)-3-hydroxy-4-(2-hydroxy-3-(trifluoromethyl)benzylamino)-1-phenylbutan-2-yl)-N3-methyl-N3-((4-methylthiazol-2-yl)methyl)isophthalamide;
3-(((2R,3S)-2-hydroxy-3-(3-(methyl((4-methylthiazol-2-yl)methyl)carbamoyl)benzamido)-4-phenylbutylamino)methyl)-5-isopropylphenyl acetate;
N1-((2S,3R)-3-hydroxy-4-((5-isopropylpyridin-3-yl)methylamino)-1-phenylbutan-2-yl)-4-methyl-N3-((4-methylthiazol-2-yl)methyl)isophthalamide;
N3-((2S,3R)-3-hydroxy-4-((5-isopropylpyridin-3-yl)methylamino)-1-phenylbutan-2-yl)-N1,4-dimethyl-N1-((4-methylthiazol-2-yl)methyl)isophthalamide;
N1-((2S,3R)-3-hydroxy-4-(3-isopropyl-5-(N-methylsulfamoyl)benzylamino)-1-phenylbutan-2-yl)-N3-methyl-N3-((4-methylthiazol-2-yl)methyl)isophthalamide;
3-(((2R,3S)-2-hydroxy-3-(3-(methyl((4-methylthiazol-2-yl)methyl)carbamoyl)benzamido)-4-phenylbutylamino)methyl)-5-isopropylphenyl dimethyl phosphate;
5-acetyl-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide;
5-(furan-2-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-N3-methyl-5-(1-methyl-1H-pyrrol-2-yl)-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-N3-methyl-5-(1-methyl-1H-pyrazol-4-yl)-N3-((4-methylthiazol-2-yl)methyl)isophthalamide;
5-(6-fluoropyridin-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
5-(furan-3-yl)-N1-((2S,3R)-3-hydroxy-4-((5-isopropylpyridin-3-yl)methylamino)-1-phenylbutan-2-yl)-N3-methyl-N3-((4-methylthiazol-2-yl)methyl)isophthalamide;
5-(furan-2-yl)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide;
N1-((2S,3R)-4-((5-acetylpyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-N3-methyl-N3-((4-methylthiazol-2-yl)methyl)-5-(1H-pyrazol-4-yl)isophthalamide;
3-(((2R,3S)-2-hydroxy-3-(3-(methyl((4-methylthiazol-2-yl)methyl)carbamoyl)benzamido)-4-phenylbutylamino)methyl)-5-(prop-1-en-2-yl)pyridine 1-oxide;
N1-((2S,3R)-4-((5-(2-fluoropropan-2-yl)pyridin-3-yl)methylamino)-3-hydroxy-1-phenylbutan-2-yl)-5-(furan-3-yl)-N3-methyl-N3-((4-methylthiazol-2-yl)methyl)isophthalamide;
N1-((2S,3R)-3-hydroxy-4-((5-isopropylpyridin-3-yl)methylamino)-1-phenylbutan-2-yl)-N3-methyl-N3-((5-methyl-1,3,4-oxadiazol-2-yl)methyl)isophthalamide;
N1-((2S,3R)-3-hydroxy-4-(3-methoxybenzylamino)-1-phenylbutan-2-yl)-5-(3-hydroxycyclopentyl)-N3-methyl-N3-((4-methylthiazol-2-yl)methyl)isophthalamide;
5-acetyl-N1-((2S,3R)-1-(3,5-difluorophenyl)-3-hydroxy-4-(3-methoxybenzylamino)butan-2-yl)-N3-((R)-1-phenylethyl)isophthalamide;
5-acetyl-N1-((2S,3R)-3-hydroxy-4-((5-isopropylpyridin-3-yl)methylamino)-1-phenylbutan-2-yl)-N3-((R)-1-phenylethyl)isophthalamide;
N1-((2S,3R)-3-hydroxy-4-(3-methoxyphenylsulfonamido)-1-phenylbutan-2-yl)-N3-methyl-N3-((4-methylthiazol-2-yl)methyl)isophthalamide;
N1-((2S,3R)-3-hydroxy-1-phenyl-4-(3-(trifluoromethyl)phenylsulfonamido)butan-2-yl)-N3-methyl-N3-((4-methylthiazol-2-yl)methyl)isophthalamide;
3-(((2R,3S)-2-hydroxy-3-(3-(methyl((4-methylthiazol-2-yl)methyl)carbamoyl)benzamido)-4-phenylbutylamino)methyl)-5-isopropylphenyl 2-(dimethylamino)acetate;
N1-((2S,3R)-3-hydroxy-4-(((5-isopropylpyridin-3-yl)methyl)(methyl)amino)-1-phenylbutan-2-yl)-N3-methyl-N3-((4-methylthiazol-2-yl)methyl)isophthalamide;
N1-((2S,3R)-3-hydroxy-4-((5-(1-hydroxypropan-2-yl)pyridin-3-yl)methylamino)-1-phenylbutan-2-yl)-N3-methyl-N3-((4-methylthiazol-2-yl)methyl)isophthalamide;
N1-(4-bromothiazol-2-yl)methyl)-N3-((2S,3R)-3-hydroxy-4-((5-isopropylpyridin-3-yl)methylamino)-1-phenylbutan-2-yl)-N1-methylisophthalamide;
N1-((2S,3R)-3-hydroxy-4-((5-isopropylpyridin-3-yl)methylamino)-1-phenylbutan-2-yl)-N3-((4-(hydroxymethyl)thiazol-2-yl)methyl)-N3-methylisophthalamide;
tert-butyl 3′-((2S,3R)-3-hydroxy-1-phenyl-4-(3-(trifluoromethyl)benzylamino)butan-2-ylcarbamoyl)-5′-(methyl((4-methylthiazol-2-yl)methyl)carbamoyl)biphenyl-3-yl(methyl)carbamate;
N1-((2S,3R)-3-hydroxy-4-((5-isopropylpyridin-3-yl)methylamino)-1-phenylbutan-2-yl)-N3-methyl-N3-(thiazol-2-ylmethyl)isophthalamide;
N1-((2S,3R)-3-hydroxy-1-phenyl-4-((5-(prop-1-en-2-yl)pyridin-3-yl)methylamino)butan-2-yl)-N3-((4-(hydroxymethyl)thiazol-2-yl)methyl)-N3-methylisophthalamide;
N1-((2S,3R)-4-((1-ethyl-1H-pyrazol-4-yl)methylamino)-3-hydroxy-1-phenylbutan-2-yl)-N3-methyl-N3-((4-methylthiazol-2-yl)methyl)isophthalamide;
or a pharmaceutically acceptable salt or solvate thereof.

2-31. (canceled)

32. A compound of claim 1 which is:
N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide;
N1-((2S,3R)-4-(3-fluoro-5-(trifluoromethyl)benzylamino)-3-hydroxy-1-phenylbutan-2-yl)-N3-methyl-N3-((4-methylthiazol-2-yl)methyl)isophthalamide;
N1-((2S,3R)-4-(2-fluoro-6-methoxybenzylamino)-3-hydroxy-1-phenylbutan-2-yl)-N3-methyl-N3-((4-methylthiazol-2-yl)methyl)isophthalamide;
N1-((2S,3R)-4-(3,5-bis(trifluoromethyl)benzylamino)-3-hydroxy-1-phenylbutan-2-yl)-N3-methyl-N3-((4-methylthiazol-2-yl)methyl)isophthalamide;
N1-((2S,3R)-3-hydroxy-1-phenyl-4-((6-(trifluoromethyl)pyridin-3-yl)methylamino)butan-2-yl)-N3-methyl-N3-((4-methylthiazol-2-yl)methyl)isophthalamide;
N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide;
N1-((2S,3R)-4-((2-fluoro-5-isopropylpyridin-3-yl)methylamino)-3-hydroxy-1-phenylbutan-2-yl)-N3-methyl-N3-((4-methylthiazol-2-yl)methyl)isophthalamide;
N1-cyclopropyl-N3-((2S,3R)-3-hydroxy-1-phenyl-4-(3-(trifluoromethyl)benzylamino)butan-2-yl)-N1-((4-methylthiazol-2-yl)methyl)isophthalamide;
N1-cyclopropyl-N3-((2S,3R)-3-hydroxy-1-phenyl-4-(3-(trifluoromethoxy)benzylamino)butan-2-yl)-N1-((4-methylthiazol-2-yl)methyl)isophthalamide;
N1-cyclopropyl-N3-((2S,3R)-3-hydroxy-1-phenyl-4-((5-(trifluoromethyl)pyridin-3-yl)methylamino)butan-2-yl)-N1-((4-methylthiazol-2-yl)methyl)isophthalamide;
N1-((2S,3R)-3-hydroxy-1-phenyl-4-(3-(trifluoromethyl)benzylamino)butan-2-yl)-5-methoxy-N3-methyl-N3-((4-methylthiazol-2-yl)methyl)isophthalamide;
N1-((2S,3R)-4-((5-(2-fluoropropan-2-yl)pyridin-3-yl)methylamino)-3-hydroxy-1-phenylbutan-2-yl)-5-(N-methylmethylsulfonamido)-N3-((R)-1-phenylethyl)isophthalamide;
N1-((2S,3R)-3-hydroxy-1-phenyl-4-((5-(trifluoromethyl)pyridin-3-yl)methylamino)butan-2-yl)-5-(N-methylmethylsulfonamido)-N3-((R)-1-phenylethyl)isophthalamide;
N1-((2S,3R)-3-hydroxy-1-phenyl-4-(3-(trifluoromethyl)benzylamino)butan-2-yl)-N3-methyl-N3-((4-methylthiazol-2-yl)methyl)-5-(2-oxopyrrolidin-1-yl)isophthalamide;
N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-5-yl)isophthalamide;
N1-((2S,3R)-4-(3-(difluoromethyl)benzylamino)-3-hydroxy-1-phenylbutan-2-yl)-5-(N-methylmethylsulfonamido)-N3-((R)-1-phenylethyl)isophthalamide;
N1-((2S,3R)-4-(3-(difluoromethyl)benzylamino)-3-hydroxy-1-phenylbutan-2-yl)-N3-methyl-N3-((4-methylthiazol-2-yl)methyl)isophthalamide;
N1-((2S,3R)-4-(3-(1,1-difluoroethyl)benzylamino)-3-hydroxy-1-phenylbutan-2-yl)-5-(N-methylmethylsulfonamido)-N3-((R)-1-phenylethyl)isophthalamide;
N1-((2S,3R)-4-(3-(1,1-difluoroethyl)benzylamino)-3-hydroxy-1-phenylbutan-2-yl)-N3-methyl-N3-((4-methylthiazol-2-yl)methyl)isophthalamide;
N1-((2S,3R)-4-(1-(3-(difluoromethyl)phenyl)ethylamino)-3-hydroxy-1-phenylbutan-2-yl)-N3-methyl-N3-((4-methylthiazol-2-yl)methyl)isophthalamide;
N1-((2S,3R)-4-((5-(1,1-difluoroethyl)pyridin-3-yl)methylamino)-3-hydroxy-1-phenylbutan-2-yl)-N3-methyl-N3-((4-methylthiazol-2-yl)methyl)isophthalamide;
5-(dimethylamino)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide;
N1-((2S,3R)-4-(3-(1,1-difluoroethyl)benzylamino)-3-hydroxy-1-phenylbutan-2-yl)-5-(dimethylamino)-N3-methyl-N3-((4-methylthiazol-2-yl)methyl)isophthalamide;
N1-((2S,3R)-3-hydroxy-1-phenyl-4-(3-(trifluoromethyl)benzylamino)butan-2-yl)-N3-methyl-N3-((4-methylthiazol-2-yl)methyl)-5-(pyrazin-2-yl)isophthalamide;
N1-((2S,3R)-4-((5-fluoropyridin-3-yl)methylamino)-3-hydroxy-1-phenylbutan-2-yl)-N3-methyl-N3-((4-methylthiazol-2-yl)methyl)isophthalamide;
N1-((2S,3R)-3-hydroxy-1-phenyl-4-(3-(trifluoromethyl)benzylamino)butan-2-yl)-N3,5-dimethyl-N3-((4-methylthiazol-2-yl)methyl)isophthalamide;
5-(difluoromethyl)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide;
N1-((2S,3R)-4-((5-(1,1-difluoroethyl)pyridin-3-yl)methylamino)-3-hydroxy-1-phenylbutan-2-yl)-5-(difluoromethyl)-N3-methyl-N3-((4-methylthiazol-2-yl)methyl)isophthalamide;
5-(difluoromethyl)-N1-((2S,3R)-3-hydroxy-1-phenyl-4-((5-(trifluoromethyl)pyridin-3-yl)methylamino)butan-2-yl)-N3-methyl-N3-((4-methylthiazol-2-yl)methyl)isophthalamide;
N1-((2S,3R)-4-((5-(1,1-difluoroethyl)pyridin-3-yl)methylamino)-3-hydroxy-1-phenylbutan-2-yl)-5-(dimethylamino)-N3-methyl-N3-((4-methylthiazol-2-yl)methyl)isophthalamide;
5-(dimethylamino)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide;
N1-((2S,3R)-3-hydroxy-1-phenyl-4-(3-(trifluoromethyl)benzylamino)butan-2-yl)-5-isopropyl-N3-methyl-N3-((4-methylthiazol-2-yl)methyl)isophthalamide;
N1-((2S,3R)-4-((5-(2-fluoropropan-2-yl)pyridin-3-yl)methylamino)-3-hydroxy-1-phenylbutan-2-yl)-5-isopropyl-N3-methyl-N3-((4-methylthiazol-2-yl)methyl)isophthalamide;
N1-(4-(fluoromethyl)thiazol-2-yl)methyl)-N3-((2S,3R)-3-hydroxy-1-phenyl-4-((5-(trifluoromethyl)pyridin-3-yl)methylamino)butan-2-yl)-N1-methylisophthalamide;
N1-(4-(fluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide;
N1-(4-(difluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide;
N1-((2S,3R)-3-hydroxy-1-phenyl-4-(3-(trifluoromethyl)benzylamino)butan-2-yl)-N3-methyl-N3-((4-methylthiazol-2-yl)methyl)-5-(oxazol-2-yl)isophthalamide;
N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)-5-(1H-pyrrol-1-yl)isophthalamide;
N1-((2S,3R)-4-(5-chloro-2-fluorobenzylamino)-3-hydroxy-1-phenylbutan-2-yl)-N3-methyl-N3-((4-methylthiazol-2-yl)methyl)isophthalamide;
N1-((2S,3R)-3-hydroxy-1-phenyl-4-((4-(trifluoromethyl)pyridin-2-yl)methylamino)butan-2-yl)-N3-methyl-N3-((4-methylthiazol-2-yl)methyl)isophthalamide;
N1-((2S,3R)-4-(3-bromo-5-(trifluoromethyl)benzylamino)-3-hydroxy-1-phenylbutan-2-yl)-N3-methyl-N3-((4-methylthiazol-2-yl)methyl)isophthalamide;
N1-((2S,3R)-4-(3-cyano-5-(trifluoromethyl)benzylamino)-3-hydroxy-1-phenylbutan-2-yl)-N3-methyl-N3-((4-methylthiazol-2-yl)methyl)isophthalamide;
N1-((2S,3R)-3-hydroxy-1-phenyl-4-(3-(pyridin-4-yl)-5-(trifluoromethyl)benzylamino)butan-2-yl)-N3-methyl-N3-((4-methylthiazol-2-yl)methyl)isophthalamide;
N1-((2S,3R)-4-(3-acetamido-5-(trifluoromethyl)benzylamino)-3-hydroxy-1-phenylbutan-2-yl)-N3-methyl-N3-((4-methylthiazol-2-yl)methyl)isophthalamide;
N1-((2S,3R)-3-hydroxy-4-(2-methoxy-5-(trifluoromethyl)benzylamino)-1-phenylbutan-2-yl)-N3-methyl-N3-((4-methylthiazol-2-yl)methyl)isophthalamide;
N1-((2S,3R)-3-hydroxy-4-(3-(methylamino)-5-(trifluoromethyl)benzylamino)-1-phenylbutan-2-yl)-N3-methyl-N3-((4-methylthiazol-2-yl)methyl)isophthalamide;
N1-((2S,3R)-3-hydroxy-4-(2-hydroxy-3-(trifluoromethyl)benzylamino)-1-phenylbutan-2-yl)-N3-methyl-N3-((4-methylthiazol-2-yl)methyl)isophthalamide;
5-acetyl-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide;
5-(furan-2-yl)-N1-((2S,3R)-3-hydroxy-1-phenyl-4-(3-(trifluoromethyl)benzylamino)butan-2-yl)-N3-methyl-N3-((4-methylthiazol-2-yl)methyl)isophthalamide;
N1-((2S,3R)-4-((5-(2-fluoropropan-2-yl)pyridin-3-yl)methylamino)-3-hydroxy-1-phenylbutan-2-yl)-5-(furan-3-yl)-N3-methyl-N3-((4-methylthiazol-2-yl)methyl)isophthalamide;
N1-((2S,3R)-3-hydroxy-1-phenyl-4-(3-(trifluoromethyl)phenylsulfonamido)butan-2-yl)-N3-methyl-N3-((4-methylthiazol-2-yl)methyl)isophthalamide;
tert-butyl 3′-((2S,3R)-3-hydroxy-1-phenyl-4-(3-(trifluoromethyl)benzylamino)butan-2-ylcarbamoyl)-5′-(methyl((4-methylthiazol-2-yl)methyl)carbamoyl)biphenyl-3-yl(methyl)carbamate;
or a pharmaceutically acceptable salt or solvate thereof.

33. A compound of claim 1 which is:
N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide;
N1-((2S,3R)-4-((5-(2-fluoropropan-2-yl)pyridin-3-yl)methylamino)-3-hydroxy-1-phenylbutan-2-yl)-5-(N-methylmethylsulfonamido)-N3-((R)-1-phenylethyl)isophthalamide;
5-(dimethylamino)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide;
5-(difluoromethyl)-N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)isophthalamide;
N1-((2S,3R)-4-((5-(2-fluoropropan-2-yl)pyridin-3-yl)methylamino)-3-hydroxy-1-phenylbutan-2-yl)-5-isopropyl-N3-methyl-N3-((4-methylthiazol-2-yl)methyl)isophthalamide;
N1-(4-(fluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide;
N1-(4-(difluoromethyl)thiazol-2-yl)methyl)-N3-((2S,3R)-4-((5-(2-fluoropropan-2-yl)pyridin-3-yl)methylamino)-3-hydroxy-1-phenylbutan-2-yl)-N1-methylisophthalamide;
N1-((2S,3R)-4-((5-(2-fluoropropan-2-yl)pyridin-3-yl)methylamino)-3-hydroxy-1-phenylbutan-2-yl)-N3-methyl-N3-((4-methylthiazol-2-yl)methyl)-5-(1H-pyrrol-1-yl)isophthalamide;
N1-((2S,3R)-4-((5-(2-fluoropropan-2-yl)pyridin-3-yl)methylamino)-3-hydroxy-1-phenylbutan-2-yl)-5-(furan-3-yl)-N3-methyl-N3-((4-methylthiazol-2-yl)methyl)isophthalamide;
or a pharmaceutically acceptable salt or solvate thereof.

34. A formulation comprising a compound of claim 1 and a pharmaceutically acceptable carrier.

35. A method of treating Alzheimer's disease in an individual in need thereof, the method comprising administering to the individual an effective amount of a compound of claim 1.

36. A method of reducing memapsin 2 catalytic activity, the method comprising contacting a memapsin 2 protein with an effective amount of a compound of claim 1.

37. The method of claim 36, wherein said memapsin 2 beta-secretase is contacted in a cell.

38. A method of selectively reducing memapsin 2 catalytic activity relative to memapsin 1 catalytic activity, the method comprising contacting a memapsin 2 protein with an effective amount of a compound of claim 1 in the presence of memapsin 1 beta-secretase.

39. A method of selectively reducing memapsin 2 catalytic activity relative to cathepsin D catalytic activity, the method comprising contacting a memapsin 2 protein with a therapeutically effective amount of a compound of claim 1 in the presence of cathepsin D.

40. A kit for the treatment or prevention of Alzheimer's disease in an individual, comprising:
(a) a compound of claim 1; and
(b) packaging.
